(19) United States
(12) Patent Application Publication
(10) Pub. No.: US 2025/0388543 A1
(43) Pub. Date: Dec. 25, 2025

(54) HETEROARYL COMPOUNDS FOR THE TREATMENT OF PAIN

(71) Applicant: VERTEX PHARMACEUTICALS INCORPORATED, Boston, MA (US)

(72) Inventors: Mark Thomas MILLER, Rancho Santa Fe, CA (US); Dennis James HURLEY, San Marcos, CA (US); Timothy Donald NEUBERT, San Diego, CA (US); Vijayalaksmi ARUMUGAM, San Marcos, CA (US); Sara Sabina HADIDA RUAH, La Jolla, CA (US); Jason McCARTNEY, Cardiff by the Sea, CA (US); Jinglan ZHOU, San Diego, CA (US); Jaclyn CHAU, San Diego, CA (US); Robert Martin DEMORET, San Diego, CA (US); Senait G. GHIRMAI, San Diego, CA (US); Roman Askatovich VALIULIN, San Diego, CA (US); Alexander Frederik KINTZER, Carlsbad, CA (US); David Robert SLOCHOWER, San Diego, CA (US); Kathleen AERTGEERTS, San Diego, CA (US); Elizabeth Mary BECK, Abingdon, Oxfordshire (GB); James Jun Bon MUI, Watford, Hertfordshire (GB); Miranda Adele WRIGHT, Oxford, Oxfordshire (GB); Ronald Marcellus Alphonsus KNEGTEL, Abingdon, Oxfordshire (GB); Ewa Iwona CHUDYK, Wantage, Oxfordshire (GB); Joanne Louise PINDER, Didcot, Oxfordshire (GB); Reece JACQUES, Oxford, Oxfordshire (GB); James DODD, Wallingford, Oxfordshire (GB); Iain SIMPSON, Oxford, Oxfordshire (GB)

(73) Assignee: VERTEX PHARMACEUTICALS INCORPORATED, Boston, MA (US)

(21) Appl. No.: 18/858,148

(22) PCT Filed: Apr. 21, 2023
(86) PCT No.: PCT/US2023/019478

§ 371 Date: Oct. 18, 2024

Related U.S. Application Data
(60) Provisional application No. 63/333,881, filed on Apr. 22, 2022.

(51) Int. Cl. C07D 213/80; A61K 31/4418; A61K 31/443; A61K 31/4433; A61K 31/4439; A61K 31/444; A61K 31/4709; A61K 31/497; A61K 31/498; A61K 31/501; A61K 31/506; A61K 31/513; A61K 31/5377; A61K 31/55; A61K 31/553; C07B 59/00; C07D 213/68; C07D 213/71; C07D 239/36; C07D 401/04; C07D 401/14; C07D 405/04; C07D 413/04; C07D 413/14; C07D 487/08; C07D 491/048; C07D 495/04

(52) U.S. Cl. CPC C07D 213/80; A61K 31/4418; A61K 31/443; A61K 31/4433; A61K 31/4439; A61K 31/444; A61K 31/4709; A61K 31/497; A61K 31/498; A61K 31/501; A61K 31/506; A61K 31/513; A61K 31/5377; A61K 31/55; A61K 31/553; C07B 59/002; C07D 213/68; C07D 213/71; C07D 239/36; C07D 401/04; C07D 401/14; C07D 405/04; C07D 413/04; C07D 413/14; C07D 487/08; C07D 491/048; C07D 495/04

(57) ABSTRACT

Compounds, and pharmaceutically acceptable salts thereof, useful as inhibitors of sodium channels are provided. Also provided are pharmaceutical compositions comprising the compounds or pharmaceutically acceptable salts and methods of using the compounds, pharmaceutically acceptable salts, and pharmaceutical compositions in the treatment of various disorders, including pain.

### CROSS-REFERENCE TO RELATED APPLICATIONS

This application claims the benefit of U.S. Provisional Application No. 63/333,881, filed Apr. 22, 2022, which is incorporated by reference in its entirety.

### BACKGROUND

Pain is a protective mechanism that allows healthy animals to avoid tissue damage and to prevent further damage to injured tissue. Nonetheless there are many conditions where pain persists beyond its usefulness, or where patients would benefit from inhibition of pain. Neuropathic pain is a form of chronic pain caused by an injury to the sensory nerves (Dieleman, J. P., et al., Incidence rates and treatment of neuropathic pain conditions in the general population. Pain, 2008. 137(3): p. 681-8). Neuropathic pain can be divided into two categories, pain caused by generalized metabolic damage to the nerve and pain caused by a discrete nerve injury. The metabolic neuropathies include post-herpetic neuropathy, diabetic neuropathy, and drug-induced neuropathy. Discrete nerve injury indications include post-amputation pain, post-surgical nerve injury pain, and nerve entrapment injuries like neuropathic back pain.

Voltage-gated sodium channels (Na_{V}s) are involved in pain signaling. Na_{V}s are biological mediators of electrical signaling as they mediate the rapid upstroke of the action potential of many excitable cell types (e.g. neurons, skeletal myocytes, cardiac myocytes). The evidence for the role of these channels in normal physiology, the pathological states arising from mutations in sodium channel genes, preclinical work in animal models, and the clinical pharmacology of known sodium channel modulating agents all point to the central role of Nays in pain sensation (Rush, A. M. and T. R. Cummins, Painful Research: Identification of a Small-Molecule Inhibitor that Selectively Targets Na_{V}1.8 Sodium Channels. Mol. Interv., 2007. 7(4): p. 192-5); England, S., Voltage-gated sodium channels: the search for subtype-selective analgesics. Expert Opin. Investig. Drugs 17 (12), p. 1849-64 (2008); Krafte, D. S. and Bannon, A. W., Sodium channels and nociception: recent concepts and therapeutic opportunities. Curr. Opin. Pharmacol. 8 (1), p. 50-56 (2008)). Na_{V}s mediate the rapid upstroke of the action potential of many excitable cell types (e.g. neurons, skeletal myocytes, cardiac myocytes), and thus are involved in the initiation of signaling in those cells (Hille, Bertil, Ion Channels of Excitable Membranes, Third ed. (Sinauer Associates, Inc., Sunderland, MA, 2001)). Because of the role Na_{V}s play in the initiation and propagation of neuronal signals, antagonists that reduce Na_{V} currents can prevent or reduce neural signaling and Na_{V} channels have been considered likely targets to reduce pain in conditions where hyper-excitability is observed (Chahine, M., Chatelier, A., Babich, O., and Krupp, J. J., Voltage-gated sodium channels in neurological disorders. CNS Neurol. Disord. Drug Targets 7 (2), p. 144-58 (2008)). Several clinically useful analgesics have been identified as inhibitors of Nay channels. The local anesthetic drugs such as lidocaine block pain by inhibiting Nay channels, and other compounds, such as carbamazepine, lamotrigine, and tricyclic antidepressants that have proven effective at reducing pain have also been suggested to act by sodium channel inhibition (Soderpalm, B., Anticonvulsants: aspects of their mechanisms of action. Eur. J Pain 6 Suppl. A, p. 3-9 (2002); Wang, G. K., Mitchell, J., and Wang, S. Y., Block of persistent late Na⁺ currents by antidepressant sertraline and paroxetine. J. Membr. Biol. 222 (2), p. 79-90 (2008)).

The Na_{V}s form a subfamily of the voltage-gated ion channel super-family and comprises 9 isoforms, designated Na_{V}1.1-Na_{V}1.9. The tissue localizations of the nine isoforms vary. Na_{V}1.4 is the primary sodium channel of skeletal muscle, and Na_{V}1.5 is primary sodium channel of cardiac myocytes. Na_{V}s 1.7, 1.8 and 1.9 are primarily localized to the peripheral nervous system, while Na_{V}s 1.1, 1.2, 1.3, and 1.6 are neuronal channels found in both the central and peripheral nervous systems. The functional behaviors of the nine isoforms are similar but distinct in the specifics of their voltage-dependent and kinetic behavior (Catterall, W. A., Goldin, A. L., and Waxman, S. G., International Union of Pharmacology. XLVII. Nomenclature and structure-function relationships of voltage-gated sodium channels. Pharmacol. Rev. 57 (4), p. 397 (2005)).

Upon their discovery, Na_{V}1.8 channels were identified as likely targets for analgesia (Akopian, A. N., L. Sivilotti, and J. N. Wood, A tetrodotoxin-resistant voltage-gated sodium channel expressed by sensory neurons. Nature, 1996. 379(6562): p. 257-62). Since then, Na_{V}1.8 has been shown to be a carrier of the sodium current that maintains action potential firing in small dorsal root ganglia (DRG) neurons (Blair, N. T. and B. P. Bean, Roles of tetrodotoxin (TTX)-sensitive Na⁺ current, TTX-resistant Na⁺ current, and Ca²⁺ current in the action potentials of nociceptive sensory neurons. J Neurosci., 2002. 22(23): p. 10277-90). Na_{V}1.8 is involved in spontaneous firing in damaged neurons, like those that drive neuropathic pain (Roza, C., et al., The tetrodotoxin-resistant Na⁻ channel Na_{V}1.8 is essential for the expression of spontaneous activity in damaged sensory axons of mice. J. Physiol., 2003. 550(Pt 3): p. 921-6; Jarvis, M. F., et al., A-803467, a potent and selective Na_{V}1.8 sodium channel blocker, attenuates neuropathic and inflammatory pain in the rat. Proc. Natl. Acad. Sci. USA, 2007. 104(20): p. 8520-5; Joshi, S. K., et al., Involvement of the TTX-resistant sodium channel Na_{V}1.8 in inflammatory and neuropathic, but not post-operative, pain states. Pain, 2006. 123(1-2): pp. 75-82; Lai, J., et al., Inhibition of neuropathic pain by decreased expression of the tetrodotoxin-resistant sodium channel, Na_{V}1.8. Pain, 2002. 95(1-2): p. 143-52; Dong, X. W., et al., Small interfering RNA-mediated selective knockdown of Na_{V}1.8 tetrodotoxin-resistant sodium channel reverses mechanical allodynia in neuropathic rats. Neuroscience, 2007. 146(2): p. 812-21; Huang, H. L., et al., Proteomic profiling of neuromas reveals alterations in protein composition and local protein synthesis in hyper-excitable nerves. Mol. Pain, 2008. 4: p. 33; Black, J. A., et al., Multiple sodium channel isoforms and mitogen-activated protein kinases are present in painful human neuromas. Ann. Neurol., 2008. 64(6): p. 644-53; Coward, K., et al., Immunolocalization of SNS/PN3 and NaN/SNS2 sodium channels in human pain states. Pain, 2000. 85(1-2): p. 41-50; Yiangou, Y., et al., SNS/PN3 and SNS2/NaN sodium channel-like immunoreactivity in human adult and neonate injured sensory nerves. FEBS Lett., 2000. 467(2-3): p. 249-52; Ruangsri, S., et al., Relationship of axonal voltage-gated sodium channel 1.8 (Na_{V}1.8) mRNA accumulation to sciatic nerve injury-induced painful neuropathy in rats. J. Biol. Chem. 286(46): p. 39836-47). The small DRG neurons where Na_{V}1.8 is expressed include the nociceptors involved in pain signaling. Na_{V}1.8 mediates large amplitude action potentials in small neurons of the dorsal root ganglia (Blair, N. T. and B. P. Bean, Roles of tetrodotoxin (TTX)-sensitive Na⁺ current, TTX-resistant Na⁺ current, and Ca²⁺ current in the action potentials of nociceptive sensory neurons. J. Neurosci., 2002. 22(23): p. 10277-90). Na_{V}1.8 is necessary for rapid repetitive action potentials in nociceptors, and for spontaneous activity of damaged neurons. (Choi, J. S. and S. G. Waxman, Physiological interactions between Na_{V}1.7 and Na_{V}1.8 sodium channels: a computer simulation study. J. Neurophysiol. 106(6): p. 3173-84; Renganathan, M., T. R. Cummins, and S. G. Waxman, Contribution of Na(_{V})1.8 sodium channels to action potential electrogenesis in DRG neurons. J. Neurophysiol., 2001. 86(2): p. 629-40; Roza, C., et al., The tetrodotoxin-resistant Na⁺ channel Na_{V}1.8 is essential for the expression of spontaneous activity in damaged sensory axons of mice. J. Physiol., 2003. 550(Pt 3): p. 921-6). In depolarized or damaged DRG neurons, Na_{V}1.8 appears to be a driver of hyper-excitablility (Rush, A. M., et al., A single sodium channel mutation produces hyper- or hypoexcitability in different types of neurons. Proc. Natl. Acad. Sci. USA, 2006. 103(21): p. 8245-50). In some animal pain models, Na_{V}1.8 mRNA expression levels have been shown to increase in the DRG (Sun, W., et al., Reduced conduction failure of the main axon of polymodal nociceptive C-fibers contributes to painful diabetic neuropathy in rats. Brain, 135(Pt 2): p. 359-75; Strickland, I. T., et al., Changes in the expression of Na_{V}1.7, Na_{V}1.8 and Na_{V}1.9 in a distinct population of dorsal root ganglia innervating the rat knee joint in a model of chronic inflammatory joint pain. Eur. J Pain, 2008. 12(5): p. 564-72; Qiu, F., et al., Increased expression of tetrodotoxin-resistant sodium channels Na_{V}1.8 and Na_{V}1.9 within dorsal root ganglia in a rat model of bone cancer pain. Neurosci. Lett., 512(2): p. 61-6).

The inventors have discovered that some voltage-gated sodium channel inhibitors have limitations as therapeutic agents due to, for example, a poor therapeutic window (e.g., due to a lack of Na_{V} isoform selectivity, low potency, and/or other reasons). Accordingly, there remains a need to develop selective voltage-gated sodium channel inhibitors, such as selective Na_{V}1.8 inhibitors.

### SUMMARY

In one aspect, the invention relates to a compound described herein, or a pharmaceutically acceptable salt thereof.

In another aspect, the invention relates to a pharmaceutical composition comprising the compound, or a pharmaceutically acceptable salt thereof, and one or more pharmaceutically acceptable carriers or vehicles.

In still another aspect, the invention relates to a method of inhibiting a voltage gated sodium channel in a subject by administering the compound, pharmaceutically acceptable salt, or pharmaceutical composition to the subject.

In yet another aspect, the invention relates to a method of treating or lessening the severity in a subject of a variety of diseases, disorders, or conditions, including, but not limited to, chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, and cardiac arrhythmia, by administering the compound, pharmaceutically acceptable salt, or pharmaceutical composition to the subject.

### DETAILED DESCRIPTION

In one aspect, the invention relates to a compound of formula (I) or (II)

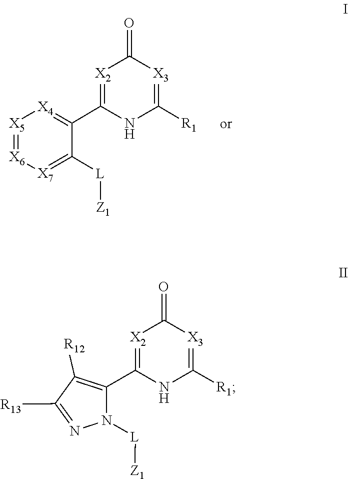

or a pharmaceutically acceptable salt thereof, wherein:
L is O, a single bond, —O—C(R)₂—, —C(R)₂—, or —C(R)₂—O—;
X₂ is N or CR₂;
X₃ is N or CR₃;
X₄ is N or CR₄;
X₅ is N or CR₅;
X₆ is N or CR₆;
X₇ is N or CR₇;
each R is independently H or C₁-C₆ alkyl;
R₁, R₂, and R₃ are defined as follows:
   (i) R₁ is H, halo, CN, OH, C₁-C₆ alkyl, C₁-C₆ alkoxy, (C₁-C₆ alkylene)-OH, NR₈R₉, or CH(OH)(CH₂)ₘ(CHOH)ₙ(CH₂)ₚH; and
      R₂ and R₃ are each independently H, halo, CN, OH, C₁-C₆ alkyl optionally substituted with C(O)OR₈, C₂-C₆ alkynyl, C₁-C₆ haloalkyl, C₁-C₆ alkoxy, C₁-C₆ haloalkoxy, (C₁-C₆ alkylene)-OH, NR₈R₉, (C₁-C₆ alkylene)-O—(C₁-C₆ alkyl), C(O)NR₈R₉, CH(OR₈)—C(O)NR₈R₉, C(O)OR₈, CHR₈—C(O)OR₉, CH(OR₈)—C(O)OR₉, CH(OH)(CH₂)ₘ(CHOH)ₙ(CH₂)ₚH, O—(C₁-C₆ alkylene)-O—CH₃, C₁-C₆ alkenyl optionally substituted with C(O)OR₈, S(O)R₈, C(O)C(O)NR₈R₉, CHR₈—C(O)NR₈R₉, C(O)R₁₀, S(═O)(═NR⁸)R⁹, S(O)₂NR₈R₉, 3-7 membered cycloalkyl, 4-7 membered heterocyclyl, or 5-6 membered heteroaryl, wherein said cycloalkyl, heterocyclyl, or heteroaryl in said 3-7 membered cycloalkyl, 4-7 membered heterocyclyl, or 5-6 membered heteroaryl is optionally substituted with 1-4 substituents independently selected from oxo, OH, C₁-C₆ alkyl, C₁-C₆ alkoxy, and C(O)NR₈R₉; or
   (ii) R₂ is H; and
      R₁ and R₃, together with the carbon atoms to which they are attached, form a ring of formula:

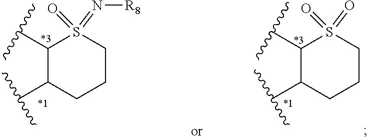

R₄, R₅, R₆, and R₇ are defined as follows:
   (i) R₄, R₅, R₆, and R₇ are each independently H, halo, C₁-C₆ alkyl, C₁-C₆ haloalkyl, C₃-C₆ cycloalkyl optionally substituted with halo;
   (ii) R₄ and R₇ are each independently is H, halo, C₁-C₆ alkyl, C₁-C₆ haloalkyl, C₃-C₆ cycloalkyl optionally substituted with halo; and R₅ and R₆, together with the carbon atoms to which they are attached, form a ring of formula:

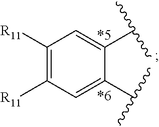

(iii) R₄ and R₇ are each independently is H, halo, C₁-C₆ alkyl, C₁-C₆ haloalkyl, C₃-C₆ cycloalkyl optionally substituted with halo; and R₅ and R₆, together with the carbon atoms to which they are attached, form a ring of formula:

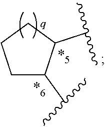

each R₈ and R₉ is independently H or C₁-C₆ alkyl;
each R₁₀ is independently C₁-C₄ alkyl;
each R₁₁ is independently H, halo, C₁-C₄ alkyl, or C₁-C₄ haloalkyl;
R₁₂ and R₁₃ are each independently H, halo, C₁-C₆ alkyl, C₁-C₆ haloalkyl, C₃-C₆ cycloalkyl optionally substituted with halo;
Z₁ is 3-10 membered cycloalkyl, 3-10 membered cycloalkenyl, phenyl, 4-10 membered heterocyclyl, or 5-6 membered heteroaryl, wherein said 3-10 membered cycloalkyl, 3-10 membered cycloalkenyl, phenyl, 4-10 membered heterocyclyl, or 5-6 membered heteroaryl may be unsubstituted or may be substituted with 1-4 substituents selected from halo, OH, CH₂OH, C₁-C₆ alkyl, C₁-C₆ alkoxy, C₁-C₆ haloalkyl, and C₁-C₆ haloalkoxy;
m, n, and p are each independently 0 or 1; and
q is 1, 2, or 3;
wherein when X₃ is CR₃, and R₃ is C(O)OR₈, then:
   L is O, or
   L is a bond, and X₅ is N, or
   L is a bond, and X₇ is N;
wherein when X₂ or X₃ is N, then:
   L is O, and Z₁ is phenyl, wherein said phenyl may be unsubstituted or may be substituted with 1-4 substituents selected from halo, OH, C₁-C₆ alkyl, C₁-C₆ alkoxy, C₁-C₆ haloalkyl, and C₁-C₆ haloalkoxy, or
   L is a single bond, and Z₁ is 4-10 membered heterocyclyl, wherein said 4-10 membered heterocyclyl may be unsubstituted or may be substituted with 1-4 substituents selected from halo, OH, C₁-C₆ alkyl, C₁-C₆ alkoxy, C₁-C₆ haloalkyl, and C₁-C₆ haloalkoxy; and
wherein the compound of formula (I) is not:

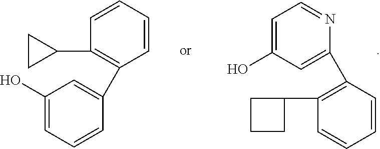 [SMILES: Oc1cccc(-c2ccccc2C2CC2)c1 ; Oc1ccnc(-c2ccccc2C2CCC2)c1]

For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001, the entire contents of which are hereby incorporated by reference.

As used herein, the term “compounds of the invention” refers to the compounds of formulas (I) and (II), and all of the embodiments thereof (e.g., formulas (I-A), etc.), as described herein, and to the compounds identified in Table A and Table B.

As described herein, the compounds of the invention comprise multiple variable groups (e.g., R₁, X₂, etc.). As one of ordinary skill in the art will recognize, combinations of groups envisioned by this invention are those combinations that result in the formation of stable or chemically feasible compounds. The term “stable,” in this context, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The chemical structures depicted herein are intended to be understood as they would be understood by one of ordinary skill in the art. For example, with respect to formulas (I) and (I-A), X₄ and X₅ are connected by a single bond, X₅ and X₆ are connected by a double bond, and X₆ and X₇ are connected by a single bond, even though the bonds between these groups may be obscured by the atom labels in the chemical structures. Using a different ChemDraw style, formula (I) could be drawn as follows to show the bonds in question:

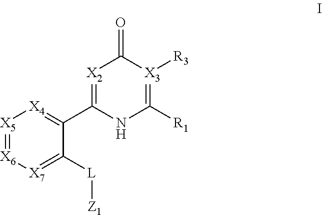

Moreover, a substituent depicted as “CF₃” or “F₃C” in a chemical structure refers to a trifluoromethyl substituent, regardless of which depiction appears in the chemical structure.

As used herein, the term “halo” means F, Cl, Br or I.

As used herein, the term “alkyl” refers to a straight or branched hydrocarbon chain radical group consisting solely of carbon and hydrogen atoms, containing no unsaturation, and having the specified number of carbon atoms, which is attached to the rest of the molecule by a single bond. For example, a “C₁-C₆ alkyl” group is an alkyl group having between one and six carbon atoms.

As used herein, the term “alkenyl” refers to a straight or branched hydrocarbon chain radical group consisting solely of carbon and hydrogen atoms, containing one or more carbon-carbon double bonds, and having the specified number of carbon atoms, which is attached to the rest of the molecule by a single bond. For example, a “C₂-C₆ alkenyl” group is an alkenyl group having between two and six carbon atoms.

As used herein, the term “alkynyl” refers to a straight or branched hydrocarbon chain radical group consisting solely of carbon and hydrogen atoms, containing one or more carbon-carbon triple bonds, and having the specified number of carbon atoms, which is attached to the rest of the molecule by a single bond. For example, a “C₂-C₆ alkynyl” group is an alkynyl group having between two and six carbon atoms.

As used herein, the term “cycloalkyl” refers to a stable, non-aromatic, mono- or bicyclic (fused, bridged, or spiro) saturated hydrocarbon radical consisting solely of carbon and hydrogen atoms, having the specified number of carbon ring atoms, and which is attached to the rest of the molecule by a single bond. For example, a “C₃-C₈ cycloalkyl” group is a cycloalkyl group having between three and eight carbon atoms.

As used herein, the term “cycloalkenyl” refers to a stable, non-aromatic, mono- or bicyclic (fused, bridged, or spiro) hydrocarbon radical consisting solely of carbon and hydrogen atoms, containing one or more carbon-carbon double bonds, and having the specified number of carbon ring atoms, which is attached to the rest of the molecule by a single bond. For example, a “C₃-C₈ cycloalkenyl” group is a cycloalkenyl group having between three and eight carbon atoms.

As used herein, the term “haloalkyl” refers to an alkyl group having the specified number of carbon atoms, wherein one or more of the hydrogen atoms of the alkyl group are replaced by halo groups. For example, a “C₁-C₆ haloalkyl” group is an alkyl group having between one and six carbon atoms, wherein one or more of the hydrogen atoms of the alkyl group are replaced by halo groups.

As used herein, the term “alkoxy” refers to a radical of the formula —ORₐ where Rₐ is an alkyl group having the specified number of carbon atoms. For example, a “C₁-C₆ alkoxy” group is a radical of the formula —ORₐ where Rₐ is an alkyl group having the between one and six carbon atoms.

As used herein, the term “haloalkoxy” refers to an alkoxy group having the specified number of carbon atoms, wherein one or more of the hydrogen atoms of the of the alkyl group are replaced by halo groups.

As used herein, the term “alkylene” refers to a divalent, straight or branched hydrocarbon chain radical group consisting solely of carbon and hydrogen atoms, containing no unsaturation, and having the specified number of carbon atoms, which is attached to the rest of the molecule by two single bonds. For example, a “C₁-C₆ alkylene” group is an alkylene group having between one and six carbon atoms.

As used herein, the term “heterocyclyl” refers to a stable, non-aromatic, mono-, bi-, or tricyclic (fused, bridged, or spiro) radical in which one or more ring atoms is a heteroatom (e.g., a heteroatom independently selected from N, O, P, and S), which has the specified number of ring atoms, which is attached to the rest of the molecule by a single bond. Heterocyclic rings can be saturated or can contain one or more double or triple bonds. In some embodiments, the “heterocyclyl” group has the indicated number of ring members, in which one or more ring members is a heteroatom independently selected from oxygen, sulfur, nitrogen, and phosphorus, and each ring in the ring system contains 3 to 7 ring members. For example, a 6-membered heterocyclyl includes a total of 6 ring members, at least one of which is a heteroatom (e.g., a heteroatom independently selected from N, O, P, and S).

As used herein, the term “heteroaryl” refers to a stable mono-, bi-, or tricyclic radical having the specified number of ring atoms, wherein at least one ring in the system is aromatic, at least one aromatic ring in the system contains one or more heteroatoms (e.g., one or more heteroatoms independently selected from N, O, P, and S). In some embodiments, each ring in the system contains 3 to 7 ring members. For example, a 6-membered heteroaryl includes a total of 6 ring members, at least one of which is a heteroatom selected from N, S, O, and P. The term “heteroaryl” may be used interchangeably with the term “heteroaryl ring” or the term “heteroaromatic”.

As used herein, the term “optionally substituted” refers to a group that is either unsubstituted or substituted with the subsequently identified substituents. For example, a group that is “optionally substituted with 1-2 halo” is either unsubstituted, substituted with 1 halo group, or substituted with 2 halo groups.

As used herein, labels such as “*5” and “*6”, such as those shown in the following structures, designate the atoms to which the corresponding R groups (in this case, the R₅ and R₆ groups, respectively) are attached.

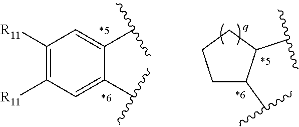

Similarly, labels such as “*3” and “*1”, such as those shown in the following structures, designate the atoms to which the corresponding R groups (in this case, the R₃ and R₁ groups, respectively) are attached.

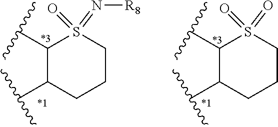

Unless otherwise specified, the compounds of the invention, whether identified by chemical name or chemical structure, include all stereoisomers (e.g., enantiomers and diastereomers), double bond isomers (e.g., (Z) and (E)), conformational isomers, and tautomers of the compounds identified by the chemical names and chemical structures provided herein. In addition, single stereoisomers, double bond isomers, conformational isomers, and tautomers as well as mixtures of stereoisomers, double bond isomers, conformational isomers, and tautomers are within the scope of the invention.

As used herein, in any chemical structure or formula, a non-bold, straight bond attached to a stereocenter of a compound, such as in

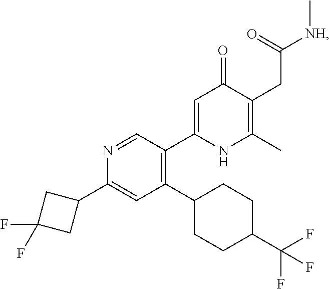 [SMILES: CNC(=O)Cc1c(C)[nH]c(-c2cnc(C3CC(F)(F)C3)cc2C2CCC(C(F)(F)F)CC2)cc1=O]

denotes that the configuration of the stereocenter is unspecified. The compound may have any configuration, or a mixture of configurations, at the stereocenter.

As used herein, in any chemical structure or formula, a bold or hashed straight bond attached to a stereocenter of a compound, such as in

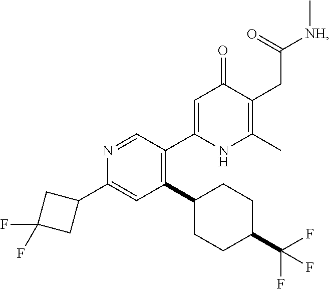

denotes the relative stereochemistry of the stereocenter, relative to other stereocenter(s) to which bold or hashed straight bonds are attached.

As used herein, in any chemical structure or formula, a bold or hashed wedge bond attached to a stereocenter of a compound, such as in

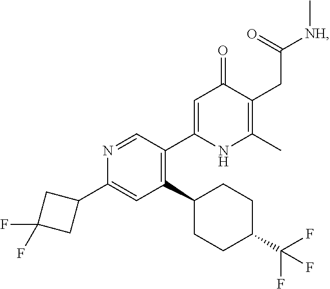 [SMILES: CNC(=O)Cc1c(C)[nH]c(-c2cnc(C3CC(F)(F)C3)cc2[C@H]2CC[C@H](C(F)(F)F)CC2)cc1=O]

denotes the absolute stereochemistry of the stereocenter, as well as the relative stereochemistry of the stereocenter, relative to other stereocenter(s) to which bold or hashed wedge bonds are attached.

As used herein, the prefix “rac-,” when used in connection with a chiral compound, refers to a racemic mixture of the compound. In a compound bearing the “rac-” prefix, the (R)- and (S)-designators in the chemical name reflect the relative stereochemistry of the compound.

As used herein, the prefix “rel-,” when used in connection with a chiral compound, refers to a single enantiomer of unknown absolute configuration. In a compound bearing the “rel-” prefix, the (R)- and (S)-designators in the chemical name reflect the relative stereochemistry of the compound, but do not necessarily reflect the absolute stereochemistry of the compound. Where the relative stereochemistry of a given stereocenter is unknown, no stereochemical designator is provided. In some instances, the absolute configuration of some stereocenters is known, while only the relative configuration of the other stereocenters is known. In these instances, the stereochemical designators associated with the stereocenters of known absolute configuration are marked with an asterisk (*), e.g., (R*)- and (S*)-, while the stereochemical designators associated with stereocenters of unknown absolute configuration are not so marked. The unmarked stereochemical designators associated with the stereocenters of unknown absolute configuration reflect the relative stereochemistry of those stereocenters with respect to other stereocenters of unknown absolute configuration, but do not necessarily reflect the relative stereochemistry with respect to the stereocenters of known absolute configuration.

As used herein, the term “compound,” when referring to the compounds of the invention, refers to a collection of molecules having identical chemical structures, except that there may be isotopic variation among the constituent atoms of the molecules. The term “compound” includes such a collection of molecules without regard to the purity of a given sample containing the collection of molecules. Thus, the term “compound” includes such a collection of molecules in pure form, in a mixture (e.g., solution, suspension, colloid, or pharmaceutical composition, or dosage form) with one or more other substances, or in the form of a hydrate, solvate, or co-crystal.

In the specification and claims, unless otherwise specified, any atom not specifically designated as a particular isotope in any compound of the invention is meant to represent any stable isotope of the specified element. In the Examples, where an atom is not specifically designated as a particular isotope in any compound of the invention, no effort was made to enrich that atom in a particular isotope, and therefore a person of ordinary skill in the art would understand that such atom likely was present at approximately the natural abundance isotopic composition of the specified element.

As used herein, the term “stable,” when referring to an isotope, means that the isotope is not known to undergo spontaneous radioactive decay. Stable isotopes include, but are not limited to, the isotopes for which no decay mode is identified in V. S. Shirley & C. M. Lederer, Isotopes Project, Nuclear Science Division, Lawrence Berkeley Laboratory, Table of Nuclides (January 1980).

As used herein in the specification and claims, “H” refers to hydrogen and includes any stable isotope of hydrogen, namely ¹H and D. In the Examples, where an atom is designated as “H,” no effort was made to enrich that atom in a particular isotope of hydrogen, and therefore a person of ordinary skill in the art would understand that such hydrogen atom likely was present at approximately the natural abundance isotopic composition of hydrogen.

As used herein, “¹H” refers to protium. Where an atom in a compound of the invention, or a pharmaceutically acceptable salt thereof, is designated as protium, protium is present at the specified position at at least the natural abundance concentration of protium.

As used herein, “D,” “d,” and “²H” refer to deuterium.

In some embodiments, the compounds of the invention, and pharmaceutically acceptable salts thereof, include each constituent atom at approximately the natural abundance isotopic composition of the specified element.

In some embodiments, the compounds of the invention, and pharmaceutically acceptable salts thereof, include one or more atoms having an atomic mass or mass number which differs from the atomic mass or mass number of the most abundant isotope of the specified element (“isotope-labeled” compounds and salts). Examples of stable isotopes which are commercially available and suitable for the invention include without limitation isotopes of hydrogen, carbon, nitrogen, oxygen, and phosphorus, for example ²H, ¹³C, ¹⁵N, ¹⁸O, ¹⁷O, and ³¹P, respectively.

The isotope-labeled compounds and salts can be used in a number of beneficial ways, including as medicaments. In some embodiments, the isotope-labeled compounds and salts are deuterium (²H)-labeled. Deuterium (²H)-labeled compounds and salts are therapeutically useful with potential therapeutic advantages over the non-²H-labeled compounds. In general, deuterium (²H)-labeled compounds and salts can have higher metabolic stability as compared to those that are not isotope-labeled owing to the kinetic isotope effect described below. Higher metabolic stability translates directly into an increased in vivo half-life or lower dosages, which under most circumstances would represent a preferred embodiment of the present invention. The isotope-labeled compounds and salts can usually be prepared by carrying out the procedures disclosed in the synthesis schemes, the examples and the related description, replacing a non-isotope-labeled reactant by a readily available isotope-labeled reactant.

The deuterium (²H)-labeled compounds and salts can manipulate the rate of oxidative metabolism of the compound by way of the primary kinetic isotope effect. The primary kinetic isotope effect is a change of the rate for a chemical reaction that results from exchange of isotopic nuclei, which in turn is caused by the change in ground state energies of the covalent bonds involved in the reaction. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For example, if deuterium is bonded to a carbon atom at a non-exchangeable position, rate differences of k_{H}/k_{D}=2-7 are typical. For a further discussion, see S. L. Harbeson and R. D. Tung, Deuterium In Drug Discovery and Development, Ann. Rep. Med. Chem. 2011, 46, 403-417, incorporated in its entirety herein by reference.

The concentration of an isotope (e.g., deuterium) incorporated at a given position of an isotope-labeled compound of the invention, or a pharmaceutically acceptable salt thereof, may be defined by the isotopic enrichment factor. The term “isotopic enrichment factor,” as used herein, means the ratio between the abundance of an isotope at a given position in an isotope-labeled compound (or salt) and the natural abundance of the isotope.

Where an atom in a compound of the invention, or a pharmaceutically acceptable salt thereof, is designated as deuterium, such compound (or salt) has an isotopic enrichment factor for such atom of at least 3000 (˜45% deuterium incorporation). In some embodiments, the isotopic enrichment factor is at least 3500 (˜52.5% deuterium incorporation), at least 4000 (˜60% deuterium incorporation), at least 4500 (˜67.5% deuterium incorporation), at least 5000 (˜75% deuterium incorporation), at least 5500 (˜82.5% deuterium incorporation), at least 6000 (˜90% deuterium incorporation), at least 6333.3 (˜95% deuterium incorporation), at least 6466.7 (˜97% deuterium incorporation), at least 6600 (˜99% deuterium incorporation), or at least 6633.3 (˜99.5% deuterium incorporation).

In some embodiments, the invention relates to a compound of formula (I), or a pharmaceutically acceptable salt thereof, wherein R₁ is H, halo, CN, OH, C₁-C₆ alkyl, C₁-C₆ alkoxy, (C₁-C₆ alkylene)-OH, NR₈R₉, or CH(OH)(CH₂)ₘ(CHOH)ₙ(CH₂)ₚH; and R₂ and R₃ are each independently H, halo, CN, OH, C₁-C₆ alkyl optionally substituted with C(O)OR₈, C₂-C₆ alkynyl, C₁-C₆ haloalkyl, C₁-C₆ alkoxy, C₁-C₆ haloalkoxy, (C₁-C₆ alkylene)-OH, NR₈R₉, (C₁-C₆ alkylene)-O—(C₁-C₆ alkyl), C(O)NR₈R₉, CH(OR₈)—C(O)NR₈R₉, C(O)OR₈, CHR₈—C(O)OR₉, CH(OR₈)—C(O)OR₉, CH(OH)(CH₂)ₘ(CHOH)ₙ(CH₂)ₚH, O—(C₁-C₆ alkylene)-O—CH₃, C₁-C₆ alkenyl optionally substituted with C(O)OR₈, S(O)R₈, C(O)C(O)NR₈R₉, CHR₈—C(O)NR₈R₉, C(O)R₁₀, S(═O)(═NR⁸)R⁹, S(O)₂NR₈R₉, 3-7 membered cycloalkyl, 4-7 membered heterocyclyl, or 5-6 membered heteroaryl, wherein said cycloalkyl, heterocyclyl, or heteroaryl in said 3-7 membered cycloalkyl, 4-7 membered heterocyclyl, or 5-6 membered heteroaryl is optionally substituted with 1-4 substituents independently selected from oxo, OH, C₁-C₆ alkyl, C₁-C₆ alkoxy, and C(O)NR₈R₉.

In some embodiments, the invention relates to a compound of formula (I), or a pharmaceutically acceptable salt thereof, wherein R₁ is H, halo, CN, OH, C₁-C₆ alkyl, C₁-C₆ alkoxy, (C₁-C₆ alkylene)-OH, NR₈R₉, or CH(OH)(CH₂)ₘ(CHOH)ₙ(CH₂)ₚH; and R₂ and R₃ are each independently H, halo, CN, OH, C₁-C₆ alkyl optionally substituted with C(O)OR₈, C₂-C₆ alkynyl, C₁-C₆ haloalkyl, C₁-C₆ alkoxy, C₁-C₆ haloalkoxy, (C₁-C₆ alkylene)-OH, NR₈R₉, (C₁-C₆ alkylene)-O—(C₁-C₆ alkyl), C(O)NR₈R₉, CH(OR₈)—C(O)NR₈R₉, C(O)OR₈, CHR₈—C(O)OR₉, CH(OR₈)—C(O)OR₉, CH(OH)(CH₂)ₘ(CHOH)ₙ(CH₂)ₚH, O—(C₁-C₆ alkylene)-O—CH₃, C₁-C₆ alkenyl optionally substituted with C(O)OR₉, S(O)R₈, C(O)C(O)NR₈R₉, CHR₈—C(O)NR₈R₉, C(O)R₁₀, S(═O)(═NR⁸)R⁹, or S(O)₂NR₈R₉.

In some embodiments, the invention relates to a compound of formula (I-A),

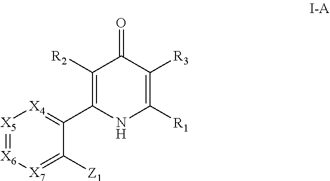

or a pharmaceutically acceptable salt thereof, wherein R₂, R₃, X₄, X₅, X₆, and X₇, are defined as set forth above in connection with formula (I);
R₁ is H, halo, CN, OH, C₁-C₆ alkyl, C₁-C₆ alkoxy, (C₁-C₆ alkylene)-OH, or —NR₈R₉;
R₄, R₅, R₆, and R₇ are each independently H, halo, C₁-C₆ alkyl, C₁-C₆ haloalkyl, C₃-C₆ cycloalkyl optionally substituted with halo; and
Z₁ is 5-10 membered cycloalkyl, phenyl, 4-10 membered heterocyclyl, or 5-6 membered heteroaryl, wherein said 5-10 membered cycloalkyl, phenyl, 4-10 membered heterocyclyl, or 5-6 membered heteroaryl may be unsubstituted or may be substituted with 1-4 substituents selected from halo, CH₂OH, C₁-C₆ alkyl, C₁-C₆ alkoxy, C₁-C₆ haloalkyl, and C₁-C₆ haloalkoxy.

In some embodiments, the invention relates to a compound of any one of formulas (I) and (I-A), or a pharmaceutically acceptable salt thereof, wherein X₄, X₅, X₆, or X₇ is N. In some embodiments, X₄ is N. In some embodiments, X₅ is N. In some embodiments, X₆ is N. In some embodiments, X₇ is N.

In some embodiments, the invention relates to a compound of any one of formulas (I) and (I-A), or a pharmaceutically acceptable salt thereof, wherein R₁ is H, halo, CN, OH, C₁-C₆ alkyl, C₁-C₆ alkoxy, (C₁-C₆ alkylene)-OH, or NR₈R₉. In some embodiments, R₁ is H. In some embodiments, R₁ is halo. In some embodiments, R₁ is CN. In some embodiments, R₁ is OH. In some embodiments, R₁ is C₁-C₆ alkyl. In some embodiments, R₁ is C₁-C₆ alkoxy. In some embodiments, R₁ is (C₁-C₆ alkylene)-OH. In some embodiments, R₁ is NR₈R₉.

In some embodiments, the invention relates to a compound of any one of formulas (I) and (I-A), or a pharmaceutically acceptable salt thereof, wherein X₂ is CR₂ and X₃ is CR₃. In some embodiments, X₂ is N. In some embodiments, X₃ is N.

In some embodiments, the invention relates to a compound of any one of formulas (I) and (I-A), or a pharmaceutically acceptable salt thereof, wherein R₂ is H, halo, CN, OH, C₁-C₆ alkyl optionally substituted with C(O)OR₈, C₂-C₆ alkynyl, C₁-C₆ haloalkyl, C₁-C₆ alkoxy, C₁-C₆ haloalkoxy, (C₁-C₆ alkylene)-OH, NR₈R₉, (C₁-C₆ alkylene)-O—(C₁-C₆ alkyl), C(O)NR₈R₉, CH(OR₈)—C(O)NR₈R₉, C(O)OR₈, CHR₈—C(O)OR₉, CH(OR₈)—C(O)OR₉, CH(OH)(CH₂)ₘ(CHOH)ₙ(CH₂)ₚH, O—(C₁-C₆ alkylene)-O—CH₃, C₁-C₆ alkenyl optionally substituted with C(O)OR₈, S(O)R₈, C(O)C(O)NR₈R₉, CHR₈—C(O)NR₈R₉, C(O)R₁₀, S(═O)(═NR⁸)R⁹, or S(O)₂NR₈R₉. In some embodiments, R₂ is H. In some embodiments, R₂ is halo. In some embodiments, R₂ is CN. In some embodiments, R₂ is OH. In some embodiments, R₂ is C₁-C₆ alkyl. In some embodiments, R₂ is C₂-C₆ alkynyl. In some embodiments, R₂ is C₁-C₆ haloalkyl. In some embodiments, R₂ is C₁-C₆ alkoxy. In some embodiments, R₂ is C₁-C₆ haloalkoxy. In some embodiments, R₂ is (C₁-C₆ alkylene)-OH. In some embodiments, R₂ is NR₈R₉. In some embodiments, R₂ is (C₁-C₆ alkylene)-O—(C₁-C₆ alkyl). In some embodiments, R₂ is C(O)NR₈R₉. In some embodiments, R₂ is C(O)OR₈. In some embodiments, R₂ is CH(OR₈)—C(O)OR₉. In some embodiments, R₂ is CH(OH)(CH₂)ₘ(CHOH)ₙ(CH₂)ₚH. In some embodiments, R₂ is O—(C₁-C₆ alkylene)-O—CH₃. In some embodiments, R₂ is C₁-C₆ alkenyl. In some embodiments, R₂ is C₁-C₆ alkenyl substituted with C(O)OR₈. In some embodiments, R₂ is S(═O)(═NH)CH₃. In some embodiments, R₂ is S(O)R₈. In some embodiments, R₂ is C(O)C(O)NR₈R₉. In some embodiments, R₂ is CHR₈—C(O)NR₈R₉. In some embodiments, R₂ is C(O)R₁₀. In some embodiments, R₂ is S(O)₂NR₈R₉. In some embodiments, R₂ is C₁-C₆ alkyl substituted with C(O)OR₈.

In some embodiments, the invention relates to a compound of any one of formulas (I) and (I-A), or a pharmaceutically acceptable salt thereof, wherein R₃ is H, halo, CN, OH, C₁-C₆ alkyl optionally substituted with C(O)OR₈, C₂-C₆ alkynyl, C₁-C₆ haloalkyl, C₁-C₆ alkoxy, C₁-C₆ haloalkoxy, (C₁-C₆ alkylene)-OH, NR₈R₉, (C₁-C₆ alkylene)-O—(C₁-C₆ alkyl), C(O)NR₈R₉, CHR₈—C(O)NR₈R₉, CH(OR₈)—C(O)NR₈R₉, C(O)OR₈, CHR₈—C(O)OR₉, CH(OR₈)—C(O)OR₉, CH(OH)(CH₂)ₘ(CHOH)ₙ(CH₂)ₚH, O—(C₁-C₆ alkylene)-O—CH₃, C₁-C₆ alkenyl optionally substituted with C(O)OR₈, S(O)R₈, C(O)C(O)NR₈R₉, CHR₈—C(O)NR₈R₉, C(O)R₁₀, S(═O)(═NR)R⁹, or S(O)₂NR₈R₉. In some embodiments, R₃ is H. In some embodiments, R₃ is halo. In some embodiments, R₃ is CN. In some embodiments, R₃ is OH. In some embodiments, R₃ is C₁-C₆ alkyl. In some embodiments, R₃ is C₂-C₆ alkynyl. In some embodiments, R₃ is C₁-C₆ haloalkyl. In some embodiments, R₃ is C₁-C₆ alkoxy. In some embodiments, R₃ is C₁-C₆ haloalkoxy. In some embodiments, R₃ is (C₁-C₆ alkylene)-OH. In some embodiments, R₃ is NR₈R₉. In some embodiments, R₃ is (C₁-C₆ alkylene)-O—(C₁-C₆ alkyl). In some embodiments, R₃ is C(O)NR₈R₉. In some embodiments, R₃ is C(O)OR₈. In some embodiments, R₃ is CH(OR₈)—C(O)OR₉. In some embodiments, R₃ is CH(OH)(CH₂)ₘ(CHOH)ₙ(CH₂)ₚH. In some embodiments, R₃ is O—(C₁-C₆ alkylene)-O—CH₃. In some embodiments, R₃ is C₁-C₆ alkenyl. In some embodiments, R₃ is C₁-C₆ alkenyl substituted with C(O)OR₈. In some embodiments, R₃ is S(═O)(═NH)CH₃. In some embodiments, R₃ is S(O)R₈. In some embodiments, R₃ is C(O)C(O)NR₈R₉. In some embodiments, R₃ is CHR₈—C(O)NR₈R₉. In some embodiments, R₃ is C(O)R₁₀. In some embodiments, R₃ is S(O)₂NR₈R₉. In some embodiments, R₃ is C₁-C₆ alkyl substituted with C(O)OR₈.

In some embodiments, the invention relates to a compound of any one of formulas (I) and (I-A), or a pharmaceutically acceptable salt thereof, wherein R₄, R₅, R₆, and R₇ are each independently H, halo, C₁-C₆ alkyl, C₁-C₆ haloalkyl, C₃-C₆ cycloalkyl optionally substituted with halo. In some embodiments, R₄, R₅, R₆, and R₇ are each independently H, halo, C₁-C₆ haloalkyl, or C₃-C₆ cycloalkyl substituted with 1-4 halo. In some embodiments, R₆ is C₃-C₆ cycloalkyl substituted with halo. In some embodiments, R₆ is cyclobutyl substituted with 1-2 halo. In some embodiments, X₄ is CR₄; X₅ is N; X₆ is CR₆; X₇ is CR₇; R₄ is H; R₆ is C₃-C₆ cycloalkyl substituted with halo; and R₇ is H.

In some embodiments, the invention relates to a compound of any one of formulas (I) and (I-A), or a pharmaceutically acceptable salt thereof, wherein Z₁ is 3-10 membered cycloalkyl, 3-10 membered cycloalkenyl, phenyl, 5-10 membered heterocyclyl, or 5-6 membered heteroaryl, wherein said 3-10 membered cycloalkyl, 3-10 membered cycloalkenyl, phenyl, 5-10 membered heterocyclyl, or 5-6 membered heteroaryl may be unsubstituted or may be substituted with 1-4 substituents selected from halo, OH, C₁-C₆ alkyl, C₁-C₆ alkoxy, C₁-C₆ haloalkyl, and C₁-C₆ haloalkoxy;

In some embodiments, the invention relates to a compound of any one of formulas (I) and (I-A), or a pharmaceutically acceptable salt thereof, wherein Z₁ is 5-10 membered cycloalkyl optionally substituted with 1-4 substituents selected from halo, CH₂OH, C₁-C₆ alkyl, C₁-C₆ alkoxy, C₁-C₆ haloalkyl, and C₁-C₆ haloalkoxy. In some embodiments, Z₁ is 5-10 membered cycloalkyl substituted with 1-4 substituents selected from halo, C₁-C₆ alkyl, C₁-C₆ alkoxy, C₁-C₆ haloalkyl, and C₁-C₆ haloalkoxy. In some embodiments, Z₁ is 5-7 membered cycloalkyl substituted with 1-4 substituents selected from halo, C₁-C₆ alkyl, C₁-C₆ alkoxy, C₁-C₆ haloalkyl, and C₁-C₆ haloalkoxy. In some embodiments, Z₁ is 6 membered cycloalkyl optionally substituted with 1-4 substituents selected from halo, C₁-C₆ alkyl, C₁-C₆ alkoxy, C₁-C₆ haloalkyl, and C₁-C₆ haloalkoxy. In some embodiments, Z₁ is 6 membered cycloalkyl substituted with 1-4 substituents selected from halo, C₁-C₆ alkyl, C₁-C₆ alkoxy, C₁-C₆ haloalkyl, and C₁-C₆ haloalkoxy. In some embodiments, Z₁ is 5-10 membered cycloalkyl optionally substituted with C₁-C₆ haloalkyl. In some embodiments, Z₁ is 5-10 membered cycloalkyl substituted with C₁-C₆ haloalkyl. In some embodiments, Z₁ is 6 membered cycloalkyl substituted with C₁-C₆ haloalkyl.

In some embodiments, the invention relates to a compound of any one of formulas (I) and (I-A), or a pharmaceutically acceptable salt thereof, wherein R_{L} is C₁-C₆ alkyl; R₂ and R₃ are each independently C₁-C₆ alkyl or C(O)OR₈; X₅ is N; R₄, R₅, R₆, and R₇ are each independently H or C₃-C₆ cycloalkyl substituted with 1-2 halo; and Z₁ is 5-10 membered cycloalkyl substituted with C₁-C₆ haloalkyl.

In some embodiments, the invention relates to a compound of any one of formulas (I) and (I-A), or a pharmaceutically acceptable salt thereof, wherein each R₈ is C₁-C₆ alkyl. In some embodiments, R₈ is C₁-C₆ alkyl and R₉ is H. In some embodiments, each R₈ and R₉ is H. In some embodiments, each R₈ and R₉ is C₁-C₆ alkyl.

In some embodiments, the invention relates to a compound of formula (I), or a pharmaceutically acceptable salt thereof, wherein:
L is O, —O—C(R)₂—, or —C(R)₂—O—.

In some embodiments, the invention relates to a compound of formula (I), or a pharmaceutically acceptable salt thereof, wherein:
L is O;
X₂ is CR₂;
X₃ is CR₃;
R₁ is C₁-C₆ alkyl.

In some embodiments, the invention relates to a compound of formula (I), or a pharmaceutically acceptable salt thereof, wherein:
L is O; and
Z₁ is phenyl, wherein said phenyl may be unsubstituted or may be substituted with 1-4 substituents selected from halo, CH₂OH, C₁-C₆ alkyl, C₁-C₆ alkoxy, C₁-C₆ haloalkyl, and C₁-C₆ haloalkoxy.

In some embodiments, the invention relates to a compound of formula (I), or a pharmaceutically acceptable salt thereof, wherein:
L is —O—C(R)₂— or —C(R)₂—O—;
X₂ is CR₂;
X₃ is CR₃; and
Z₁ is 3 membered cycloalkyl or 4-10 membered cycloalkyl, wherein said 3 membered cycloalkyl is substituted with 1-2 substituents selected from halo, CH₂OH, C₁-C₆ alkyl, C₁-C₆ alkoxy, C₁-C₆ haloalkyl, and C₁-C₆ haloalkoxy, and said 4-10 membered cycloalkyl may be unsubstituted or may be substituted with 1-4 substituents selected from halo, CH₂OH, C₁-C₆ alkyl, C₁-C₆ alkoxy, C₁-C₆ haloalkyl, and C₁-C₆ haloalkoxy.

In some embodiments, the invention relates to a compound of formula (I), or a pharmaceutically acceptable salt thereof, wherein X₂ is CR₂; X₃ is CR₃; and R₂ and R₃ are each independently H, halo, CN, OH, C₁-C₆ alkyl optionally substituted with C(O)OR₈, C₁-C₆ alkoxy, C₁-C₆ haloalkoxy, C(O)OR₈, S(═O)(═NH)CH₃, S(O)R₈, C(O)R₁₀, or S(O)₂NR₈R₉. In some embodiments, R₂ and R₃ are each independently H, halo, C(O)OR₈, S(═O)(═NH)CH₃, S(O)R₈, or S(O)₂NR₈R₉.

In some embodiments, the invention relates to a compound of formula (I), or a pharmaceutically acceptable salt thereof, wherein Z₁ is phenyl substituted with 1-3 substituents selected from halo, C₁-C₆ alkyl, C₁-C₆ alkoxy, and C₁-C₆ haloalkoxy. In some embodiments, the invention relates to a compound of formula (I), or a pharmaceutically acceptable salt thereof, wherein Z₁ is phenyl substituted 1-3 substituents selected from halo, CH₂OH, C₁-C₆ alkyl, C₁-C₆ alkoxy, and C₁-C₆ haloalkoxy.

In some embodiments, the invention relates to a compound of formula (I), or a pharmaceutically acceptable salt thereof, wherein X₂ is CR₂; X₃ is CR₃; X₄ is CR₄; X₅ is CR₅; X₆ is CR₆; and X₇ is CR₇. In some embodiments, R₄, R₅, R₆, and R₇ are each independently H, halo, C₁-C₆ alkyl, or C₁-C₆ haloalkyl.

In some embodiments, the invention relates to a compound of formula (I), or a pharmaceutically acceptable salt thereof, wherein X₅ is CR₅; X₆ is CR₆; and R₅ and R₆, together with the carbon atoms to which they are attached, form a ring of formula:

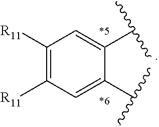

In some embodiments, the invention relates to a compound of formula (I), or a pharmaceutically acceptable salt thereof, wherein each R₁ is independently H or halo.

In some embodiments, the invention relates to a compound of formula (I), or a pharmaceutically acceptable salt thereof, wherein:
L is a single bond;
X₂ is CR₂;
X₃ is CR₃;
Z₁ is 4-10 membered heterocyclyl or 5-6 membered heteroaryl, wherein said 4-10 membered heterocyclyl or 5-6 membered heteroaryl may be unsubstituted or may be substituted with 1-4 substituents selected from halo, OH, CH₂OH, C₁-C₆ alkyl, C₁-C₆ alkoxy, C₁-C₆ haloalkyl, and C₁-C₆ haloalkoxy.

In some embodiments, the invention relates to a compound of formula (I), or a pharmaceutically acceptable salt thereof, wherein:
L is a single bond;
X₂ is CR₂;
X₃ is CR₃;
Z₁ is 5-10 membered heterocyclyl or 5-6 membered heteroaryl, wherein said 5-10 membered heterocyclyl or 5-6 membered heteroaryl may be unsubstituted or may be substituted with 1-4 substituents selected from halo, OH, C₁-C₆ alkyl, C₁-C₆ alkoxy, C₁-C₆ haloalkyl, and C₁-C₆ haloalkoxy.

In some embodiments, the invention relates to a compound of formula (I), or a pharmaceutically acceptable salt thereof, wherein:
L is a single bond;
Z₁ is a 7 membered heterocyclyl, wherein said 7 membered heterocyclyl may be unsubstituted or may be substituted with 1-4 substituents selected from halo, OH, CH₂OH, C₁-C₆ alkyl, C₁-C₆ alkoxy, C₁-C₆ haloalkyl, and C₁-C₆ haloalkoxy. In some embodiments, Z₁ is azepanyl optionally substituted with halo. In some embodiments, Z₁ is azepanyl substituted with halo.

In some embodiments, the invention relates to a compound of formula (I), or a pharmaceutically acceptable salt thereof, wherein X₂ is CR₂; X₃ is CR₃; and R₂ and R₃ are each independently H, C₁-C₆ alkoxy, or C(O)OR₈. In some embodiments, R₂ and R₃ are each independently C₁-C₆ alkoxy or C(O)OR₈.

In some embodiments, the invention relates to a compound of formula (I), or a pharmaceutically acceptable salt thereof, wherein R₂ is 3-7 membered cycloalkyl, 4-7 membered heterocyclyl, or 5-6 membered heteroaryl, wherein said cycloalkyl, heterocyclyl, or heteroaryl in said 3-7 membered cycloalkyl, 4-7 membered heterocyclyl, or 5-6 membered heteroaryl is optionally substituted with 1-4 substituents independently selected from oxo, OH, C₁-C₆ alkyl, C₁-C₆ alkoxy, and C(O)NR₈R₉; and R₃ is H, halo, CN, OH, C₁-C₆ alkyl optionally substituted with C(O)OR₈, C₂-C₆ alkynyl, C₁-C₆ haloalkyl, C₁-C₆ alkoxy, C₁-C₆ haloalkoxy, (C₁-C₆ alkylene)-OH, NR₈R₉, (C₁-C₆ alkylene)-O—(C₁-C₆ alkyl), C(O)NR₈R₉, CH(OR₈)—C(O)NR₈R₉, C(O)OR₈, CHR₈—C(O)OR₉, CH(OR₈)—C(O)OR₉, CH(OH)(CH₂)ₘ(CHOH)ₙ(CH₂)ₚH, O—(C₁-C₆ alkylene)-O—CH₃, C₁-C₆ alkenyl optionally substituted with C(O)OR₈, S(O)R₈, C(O)C(O)NR₈R₉, CHR₈—C(O)NR₈R₉, C(O)R₁₀, S(═O)(═NR⁸)R⁹, or S(O)₂NR₈R₉. In other embodiments, R₂ is 4-7 membered heterocyclyl, wherein said heterocyclyl is optionally substituted with 1-4 substituents independently selected from oxo, OH, C₁-C₆ alkyl, C₁-C₆ alkoxy, and C(O)NR₈R₉. In other embodiments. R₂ is

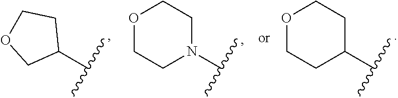 [SMILES: *C1CCOC1 ; "," ; *N1CCOCC1 ; "or" ; *C1CCOCC1 ; "."]

In other embodiments, R₂ is 5-6 membered heteroaryl, wherein said heteroaryl is optionally substituted with 1-4 substituents independently selected from oxo, OH, C₁-C₆ alkyl, C₁-C₆ alkoxy, and C(O)NR₈R₉. In other embodiments, R₂ is 5-6 membered heteroaryl, wherein said heteroaryl is

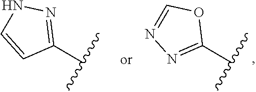 [SMILES: *c1cc[nH]n1 ; *c1nnco1]

wherein said heteroaryl is optionally substituted with 1-4 C₁-C₆ alkyl substituents. In other embodiments, R₂ is

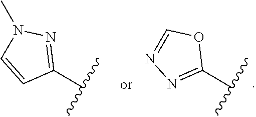

In some embodiments, the invention relates to a compound of formula (I), or a pharmaceutically acceptable salt thereof, wherein R₃ is H or C₁-C₆ alkyl.

In some embodiments, the invention relates to a compound of formula (I), or a pharmaceutically acceptable salt thereof, wherein R₂ is H, halo, CN, OH, C₁-C₆ alkyl optionally substituted with C(O)OR₈, C₂-C₆ alkynyl, C₁-C₆ haloalkyl, C₁-C₆ alkoxy, C₁-C₆ haloalkoxy, (C₁-C₆ alkylene)-OH, NR₈R₉, (C₁-C₆ alkylene)-O—(C₁-C₆ alkyl), C(O)NR₈R₉, CH(OR₈)—C(O)NR₈R₉, C(O)OR₈, CHR₈—C(O)OR₉, CH(OR₈)—C(O)OR₉, CH(OH)(CH₂)ₘ(CHOH)ₙ(CH₂)ₚH, O—(C₁-C₆ alkylene)-O—CH₃, C₁-C₆ alkenyl optionally substituted with C(O)OR₈, S(O)R₈, C(O)C(O)NR₈R₉, CHR₈—C(O)NR₈R₉, C(O)R₁₀, S(═O)(═NR⁸)R⁹, or S(O)₂NR₈R₉; and R₃ is 3-7 membered cycloalkyl, 4-7 membered heterocyclyl, or 5-6 membered heteroaryl, wherein said cycloalkyl, heterocyclyl, or heteroaryl in said 3-7 membered cycloalkyl, 4-7 membered heterocyclyl, or 5-6 membered heteroaryl is optionally substituted with 1-4 substituents independently selected from oxo, OH, C₁-C₆ alkyl, C₁-C₆ alkoxy, and C(O)NR₈R₉. In other embodiments, R₂ is H or C(O)OR₈, wherein R₈ is C₁-C₆ alkyl. In other embodiments, R₃ is 3-7 membered cycloalkyl, wherein said cycloalkyl is optionally substituted with 1-4 substituents independently selected from oxo, OH, C₁-C₆ alkyl, C₁-C₆ alkoxy, and C(O)NR₈R₉. In other embodiments, R₃ is

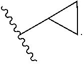 [SMILES: *C1CC1]

In other embodiments, R₃ is 4-7 membered heterocyclyl, wherein said heterocyclyl is optionally substituted with 1-4 substituents independently selected from oxo, OH, C₁-C₆ alkyl, C₁-C₆ alkoxy, and C(O)NR₈R₉. In other embodiments, R₃ is 4-7 membered heterocyclyl, wherein said heterocyclyl is

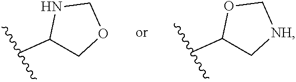 [SMILES: *C1COCN1 ; *C1CNCO1]

wherein said heterocyclyl is substituted with one oxo substituent. In other embodiments, R₃ is

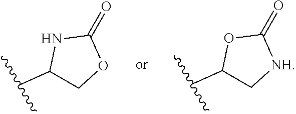 [SMILES: *C1COC(=O)N1 ; *C1CNC(=O)O1]

In other embodiments, R₃ is 5-6 membered heteroaryl, wherein said heteroaryl is optionally substituted with 1-4 substituents independently selected from oxo, OH, C₁-C₆ alkyl, C₁-C₆ alkoxy, and C(O)NR₈R₉. In other embodiments, R₃ is 5-6 membered heteroaryl, wherein said heteroaryl is

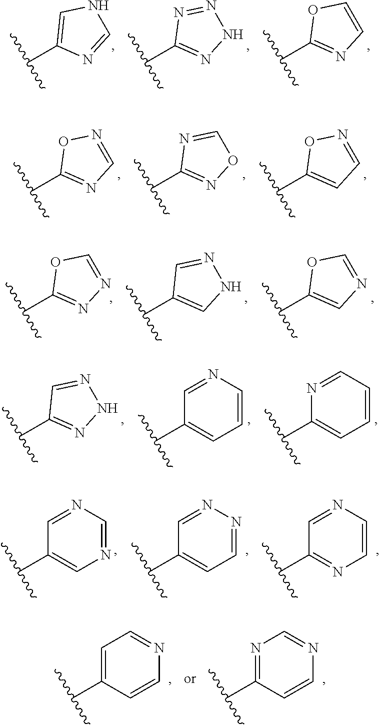

wherein said heteroaryl is optionally substituted with 1-4 substituents independently selected from oxo, OH, C₁-C₆ alkyl, C₁-C₆ alkoxy, and C(O)NR₈R₉. In other embodiments, R₃ is

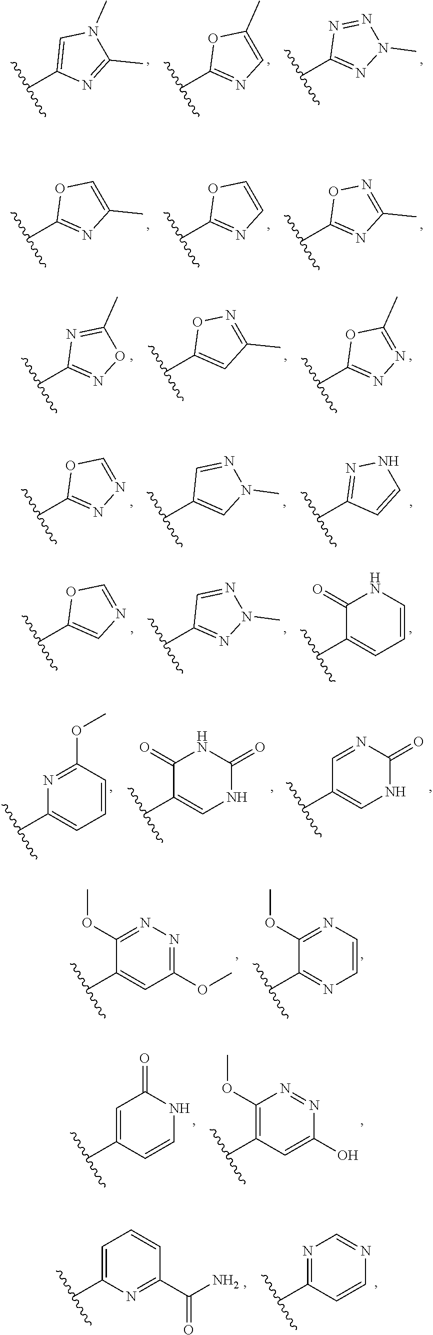

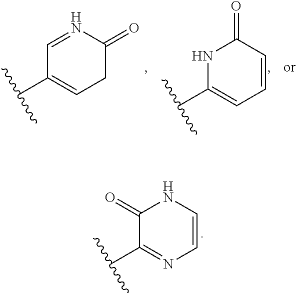

In some embodiments, the invention relates to a compound of formula (II), or a pharmaceutically acceptable salt thereof, wherein L is a single bond; X₂ is CH; X₃ is CR₃; R₁ is C₁-C₆ alkyl; R₃ is 5-6 membered heteroaryl, wherein said heteroaryl is optionally substituted with 1-4 C₁-C₆ alkyl substituents; R₄ and R₅ are each independently C₁-C₆ alkyl; and Z₁ is 3-10 membered cycloalkyl, wherein said 3-10 membered cycloalkyl may be unsubstituted or may be substituted with 1-4 C₁-C₆ alkyl substituents.

In some embodiments, the invention relates to a compound of any one of formulas (I), (II), and (I-A), or any embodiment thereof, i.e., the compound in non-salt form.

In some embodiments, the invention relates to a compound selected from Table A, or a pharmaceutically acceptable salt thereof. In other embodiments, the invention relates to a compound selected from Table A, i.e., the compound in non-salt form.

TABLE A
Compound Structures and Names

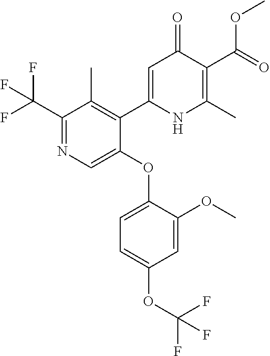 [SMILES: COC(=O)c1c(C)[nH]c(-c2c(Oc3ccc(OC(F)(F)F)cc3OC)cnc(C(F)(F)F)c2C)cc1=O]

methyl 6-[5-[2-methoxy-4-
(trifluoromethoxy)phenoxy]-3-methyl-2-
(trifluoromethyl)-4-pyridyl]-2-methyl-4-oxo-1H-
pyridine-3-carboxylate

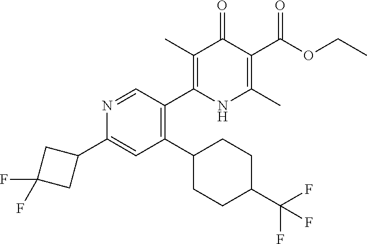 [SMILES: CCOC(=O)c1c(C)[nH]c(-c2cnc(C3CC(F)(F)C3)cc2C2CCC(C(F)(F)F)CC2)c(C)c1=O]

ethyl 6-[6-(3,3-difluorocyclobutyl)-4-[4-
(trifluoromethyl)cyclohexyl]-3-pyridyl]-2,5-
dimethyl-4-oxo-1H-pyridine-3-carboxylate

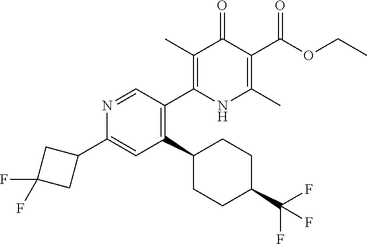 [SMILES: CCOC(=O)c1c(C)[nH]c(-c2cnc(C3CC(F)(F)C3)cc2[C@H]2CC[C@@H](C(F)(F)F)CC2)c(C)c1=O]

ethyl 6-[6-(3,3-difluorocyclobutyl)-4-[(1s,4s)-4-
(trifluoromethyl)cyclohexyl]-3-pyridyl]-2,5-
dimethyl-4-oxo-1H-pyridine-3-carboxylate

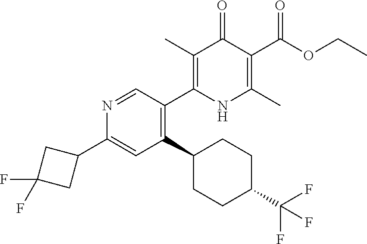 [SMILES: CCOC(=O)c1c(C)[nH]c(-c2cnc(C3CC(F)(F)C3)cc2[C@H]2CC[C@H](C(F)(F)F)CC2)c(C)c1=O]

ethyl 6-[6-(3,3-difluorocyclobutyl)-4-[(1r,4r)-4-
(trifluoromethyl)cyclohexyl]-3-pyridyl]-2,5-
dimethyl-4-oxo-1H-pyridine-3-carboxylate

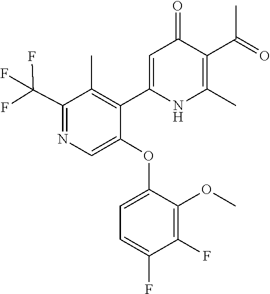 [SMILES: COc1c(Oc2cnc(C(F)(F)F)c(C)c2-c2cc(=O)c(C(C)=O)c(C)[nH]2)ccc(F)c1F]

3-acetyl-6-[5-(3,4-difluoro-2-methoxy-phenoxy)-
3-methyl-2-(trifluoromethyl)-4-pyridyl]-2-methyl-
1H-pyridin-4-one

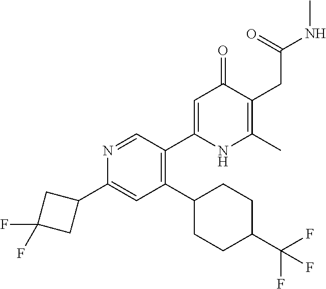 [SMILES: CNC(=O)Cc1c(C)[nH]c(-c2cnc(C3CC(F)(F)C3)cc2C2CCC(C(F)(F)F)CC2)cc1=O]

2-[6-[6-(3,3-difluorocyclobutyl)-4-[4-
(trifluoromethyl)cyclohexyl]-3-pyridyl]-2-methyl-
4-oxo-1H-pyridin-3-yl]-N-methyl-acetamide

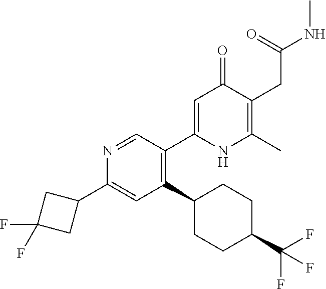 [SMILES: CNC(=O)Cc1c(C)[nH]c(-c2cnc(C3CC(F)(F)C3)cc2[C@H]2CC[C@@H](C(F)(F)F)CC2)cc1=O]

2-[6-[6-(3,3-difluorocyclobutyl)-4-((1s,4s)-4-
(trifluoromethyl)cyclohexyl)-3-pyridyl]-2-methyl-
4-oxo-1H-pyridin-3-yl]-N-methyl-acetamide

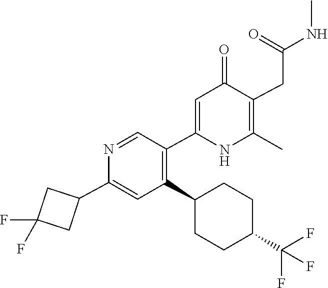 [SMILES: CNC(=O)Cc1c(C)[nH]c(-c2cnc(C3CC(F)(F)C3)cc2[C@H]2CC[C@H](C(F)(F)F)CC2)cc1=O]

2-[6-[6-(3,3-difluorocyclobutyl)-4-((1r,4r)-4-
(trifluoromethyl)cyclohexyl)-3-pyridyl]-2-methyl-
4-oxo-1H-pyridin-3-yl]-N-methyl-acetamide

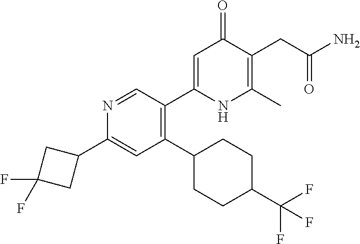 [SMILES: Cc1[nH]c(-c2cnc(C3CC(F)(F)C3)cc2C2CCC(C(F)(F)F)CC2)cc(=O)c1CC(N)=O]

2-[6-[6-(3,3-difluorocyclobutyl)-4-[4-
(trifluoromethyl)cyclohexyl]-3-pyridyl]-2-methyl-
4-oxo-1H-pyridin-3-yl]acetamide

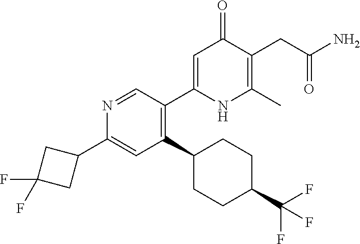 [SMILES: Cc1[nH]c(-c2cnc(C3CC(F)(F)C3)cc2[C@H]2CC[C@@H](C(F)(F)F)CC2)cc(=O)c1CC(N)=O]

2-[6-[6-(3,3-difluorocyclobutyl)-4-((1s,4s)4-
(trifluoromethyl)cyclohexyl)-3-pyridyl]-2-methyl-
4-oxo-1H-pyridin-3-yl]acetamide

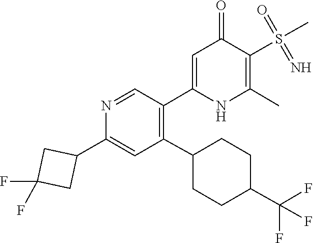 [SMILES: Cc1[nH]c(-c2cnc(C3CC(F)(F)C3)cc2C2CCC(C(F)(F)F)CC2)cc(=O)c1S(C)(=N)=O]

6-[6-(3,3-difluorocyclobutyl)-4-[4-
(trifluoromethyl)cyclohexyl]-3-pyridyl]-2-methyl-
3-(methylsulfonimidoyl)-1H-pyridin-4-one

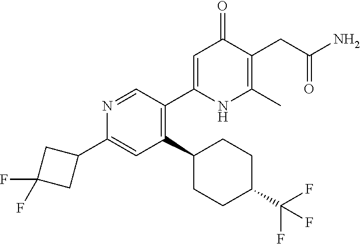 [SMILES: Cc1[nH]c(-c2cnc(C3CC(F)(F)C3)cc2[C@H]2CC[C@H](C(F)(F)F)CC2)cc(=O)c1CC(N)=O]

2-[6-[6-(3,3-difluorocyclobutyl)-4-((1r,4r)4-
(trifluoromethyl)cyclohexyl)-3-pyridyl]-2-methyl-
4-oxo-1H-pyridin-3-yl]acetamide

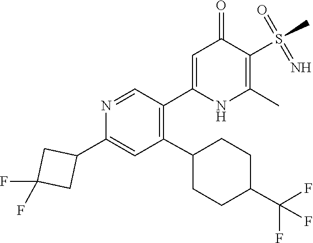 [SMILES: Cc1[nH]c(-c2cnc(C3CC(F)(F)C3)cc2C2CCC(C(F)(F)F)CC2)cc(=O)c1[S@@](C)(=N)=O]

(S)-6-[6-(3,3-difluorocyclobutyl)-4-[4-
(trifluoromethyl)cyclohexyl]-3-pyridyl]-2-methyl-
3-(methylsulfonimidoyl)-1H-pyridin-4-one

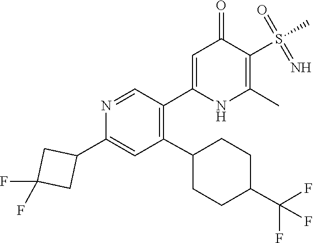 [SMILES: Cc1[nH]c(-c2cnc(C3CC(F)(F)C3)cc2C2CCC(C(F)(F)F)CC2)cc(=O)c1[S@](C)(=N)=O]

(R)-6-[6-(3,3-difluorocyclobutyl)-4-[4-
(trifluoromethyl)cyclohexyl]-3-pyridyl]-2-methyl-
3-(methylsulfonimidoyl)-1H-pyridin-4-one

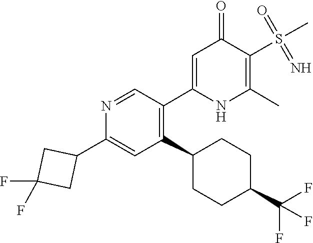 [SMILES: Cc1[nH]c(-c2cnc(C3CC(F)(F)C3)cc2[C@H]2CC[C@@H](C(F)(F)F)CC2)cc(=O)c1S(C)(=N)=O]

6-[6-(3,3-difluorocyclobutyl)-4-(1s,4s)-4-
(trifluoromethyl)cyclohexyl)-3-pyridyl]-2-methyl-
3-(methylsulfonimidoyl)-1H-pyridin-4-one

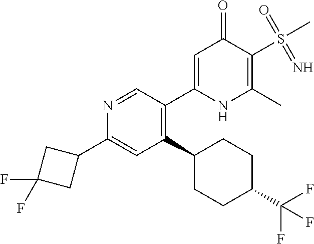 [SMILES: Cc1[nH]c(-c2cnc(C3CC(F)(F)C3)cc2[C@H]2CC[C@H](C(F)(F)F)CC2)cc(=O)c1S(C)(=N)=O]

6-[6-(3,3-difluorocyclobutyl)-4-(1r,4r)-4-
(trifluoromethyl)cyclohexyl)-3-pyridyl]-2-methyl-
3-(methylsulfonimidoyl)-1H-pyridin-4-one

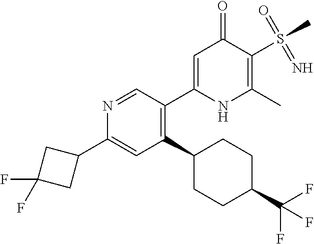 [SMILES: Cc1[nH]c(-c2cnc(C3CC(F)(F)C3)cc2[C@H]2CC[C@@H](C(F)(F)F)CC2)cc(=O)c1[S@@](C)(=N)=O]

6-[6-(3,3-difluorocyclobutyl)-4-(1s,4s)-4-
(trifluoromethyl)cyclohexyl)-3-pyridyl]-2-methyl-
3-((S)-methylsulfonimidoyl)-1H-pyridin-4-one

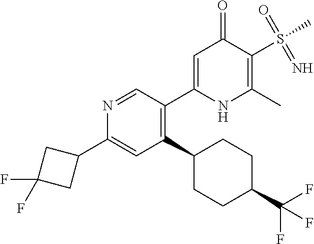 [SMILES: Cc1[nH]c(-c2cnc(C3CC(F)(F)C3)cc2[C@H]2CC[C@@H](C(F)(F)F)CC2)cc(=O)c1[S@](C)(=N)=O]

6-[6-(3,3-difluorocyclobutyl)-4-(1s,4s)-4-
(trifluoromethyl)cyclohexyl)-3-pyridyl]-2-methyl-
3-((R)-methylsulfonimidoyl)-1H-pyridin-4-one

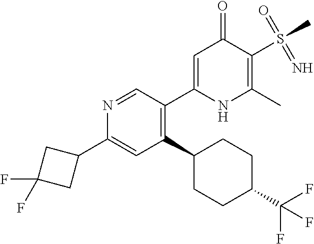 [SMILES: Cc1[nH]c(-c2cnc(C3CC(F)(F)C3)cc2[C@H]2CC[C@H](C(F)(F)F)CC2)cc(=O)c1[S@@](C)(=N)=O]

6-[6-(3,3-difluorocyclobutyl)-4-(1r,4r)-4-
(trifluoromethyl)cyclohexyl)-3-pyridyl]-2-methyl-
3-((S)-methylsulfonimidoyl)-1H-pyridin-4-one

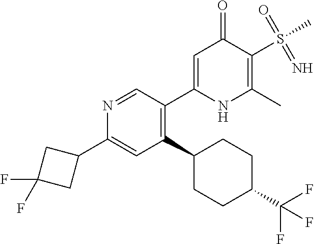 [SMILES: Cc1[nH]c(-c2cnc(C3CC(F)(F)C3)cc2[C@H]2CC[C@H](C(F)(F)F)CC2)cc(=O)c1[S@](C)(=N)=O]

6-[6-(3,3-difluorocyclobutyl)-4-(1r,4r)-4-
(trifluoromethyl)cyclohexyl)-3-pyridyl]-2-methyl-
3-((R)-methylsulfonimidoyl)-1H-pyridin-4-one

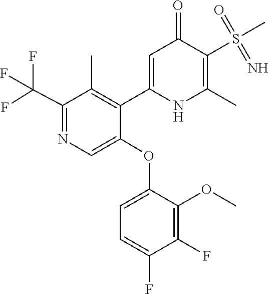 [SMILES: COc1c(Oc2cnc(C(F)(F)F)c(C)c2-c2cc(=O)c(S(C)(=N)=O)c(C)[nH]2)ccc(F)c1F]

6-[5-(3,4-difluoro-2-methoxy-phenoxy)-3-methyl-
2-(trifluoromethyl)-4-pyridyl]-2-methyl-3-
(methylsulfonimidoyl)-1H-pyridin-4-one

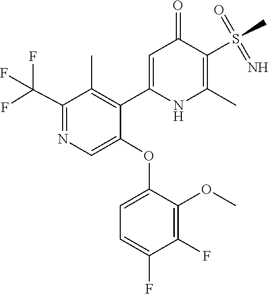 [SMILES: COc1c(Oc2cnc(C(F)(F)F)c(C)c2-c2cc(=O)c([S@@](C)(=N)=O)c(C)[nH]2)ccc(F)c1F]

(S)-6-[5-(3,4-difluoro-2-methoxy-phenoxy)-3-
methyl-2-(trifluoromethyl)-4-pyridyl]-2-methyl-3-
(methylsulfonimidoyl)-1H-pyridin-4-one

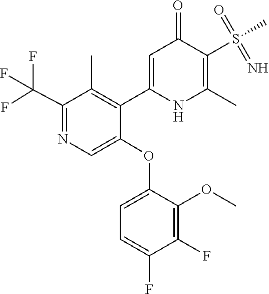 [SMILES: COc1c(Oc2cnc(C(F)(F)F)c(C)c2-c2cc(=O)c([S@](C)(=N)=O)c(C)[nH]2)ccc(F)c1F]

(R)-6-[5-(3,4-difluoro-2-methoxy-phenoxy)-3-
methyl-2-(trifluoromethyl)-4-pyridyl]-2-methyl-3-
(methylsulfonimidoyl)-1H-pyridin-4-one

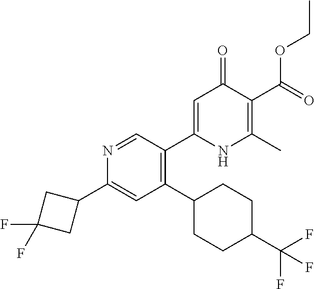 [SMILES: CCOC(=O)c1c(C)[nH]c(-c2cnc(C3CC(F)(F)C3)cc2C2CCC(C(F)(F)F)CC2)cc1=O]

ethyl 6-[6-(3,3-difluorocyclobutyl)-4-[4-
(trifluoromethyl)cyclohexyl]-3-pyridyl]-2-methyl-
4-oxo-1H-pyridine-3-carboxylate

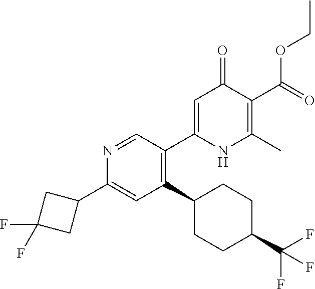 [SMILES: CCOC(=O)c1c(C)[nH]c(-c2cnc(C3CC(F)(F)C3)cc2[C@H]2CC[C@@H](C(F)(F)F)CC2)cc1=O]

ethyl 6-[6-(3,3-difluorocyclobutyl)-4-((1s,4s)-4-
(trifluoromethyl)cyclohexyl)-3-pyridyl]-2-methyl-
4-oxo-1H-pyridine-3-carboxylate

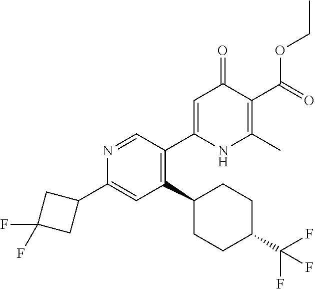 [SMILES: CCOC(=O)c1c(C)[nH]c(-c2cnc(C3CC(F)(F)C3)cc2[C@H]2CC[C@H](C(F)(F)F)CC2)cc1=O]

ethyl 6-[6-(3,3-difluorocyclobutyl)-4-((1r,4r)-4-
(trifluoromethyl)cyclohexyl)-3-pyridyl]-2-methyl-
4-oxo-1H-pyridine-3-carboxylate

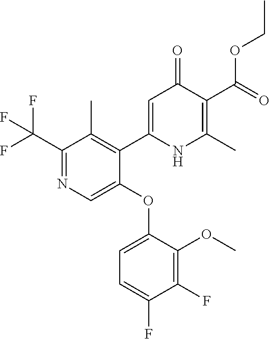 [SMILES: CCOC(=O)c1c(C)[nH]c(-c2c(Oc3ccc(F)c(F)c3OC)cnc(C(F)(F)F)c2C)cc1=O]

ethyl 6-[5-(3,4-difluoro-2-methoxy-phenoxy)-3-
methyl-2-(trifluoromethyl)-4-pyridyl]-2-methyl-4-
oxo-1H-pyridine-3-carboxylate

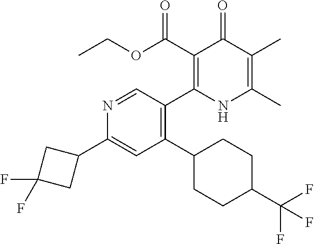 [SMILES: CCOC(=O)c1c(-c2cnc(C3CC(F)(F)C3)cc2C2CCC(C(F)(F)F)CC2)[nH]c(C)c(C)c1=O]

ethyl 2-[6-(3,3-difluorocyclobutyl)-4-(4-
(trifluoromethyl)cyclohexyl)-3-pyridyl]-5,6-
dimethyl-4-oxo-1H-pyridine-3-carboxylate

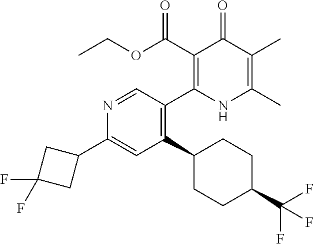 [SMILES: CCOC(=O)c1c(-c2cnc(C3CC(F)(F)C3)cc2[C@H]2CC[C@@H](C(F)(F)F)CC2)[nH]c(C)c(C)c1=O]

ethyl 2-[6-(3,3-difluorocyclobutyl)-4-((1s,4s)-4-
(trifluoromethyl)cyclohexyl)-3-pyridyl]-5,6-
dimethyl-4-oxo-1H-pyridine-3-carboxylate

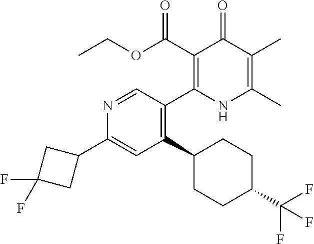 [SMILES: CCOC(=O)c1c(-c2cnc(C3CC(F)(F)C3)cc2[C@H]2CC[C@H](C(F)(F)F)CC2)[nH]c(C)c(C)c1=O]

ethyl 2-[6-(3,3-difluorocyclobutyl)-4-((1r,4r)-4-
(trifluoromethyl)cyclohexyl)-3-pyridyl]-5,6-
dimethyl-4-oxo-1H-pyridine-3-carboxylate

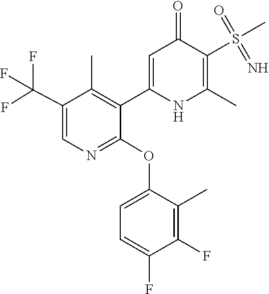 [SMILES: Cc1[nH]c(-c2c(Oc3ccc(F)c(F)c3C)ncc(C(F)(F)F)c2C)cc(=O)c1S(C)(=N)=O]

6-[2-(3,4-difluoro-2-methyl-phenoxy)-4-methyl-
5-(trifluoromethyl)-3-pyridyl]-2-methyl-3-
(methylsulfonimidoyl)-1H-pyridin-4-one

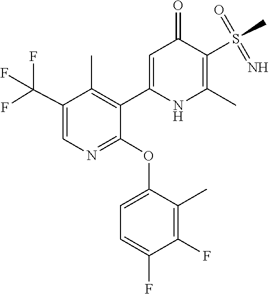 [SMILES: Cc1[nH]c(-c2c(Oc3ccc(F)c(F)c3C)ncc(C(F)(F)F)c2C)cc(=O)c1[S@@](C)(=N)=O]

(S)-6-[2-(3,4-difluoro-2-methyl-phenoxy)-4-
methyl-5-(trifluoromethyl)-3-pyridyl]-2-methyl-3-
(methylsulfonimidoyl)-1H-pyridin-4-one

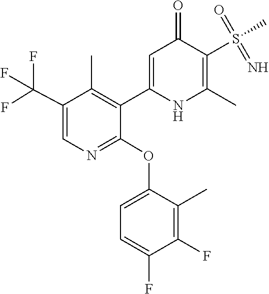 [SMILES: Cc1[nH]c(-c2c(Oc3ccc(F)c(F)c3C)ncc(C(F)(F)F)c2C)cc(=O)c1[S@](C)(=N)=O]

(R)-6-[2-(3,4-difluoro-2-methyl-phenoxy)-4-
methyl-5-(trifluoromethyl)-3-pyridyl]-2-methyl-3-
(methylsulfonimidoyl)-1H-pyridin-4-one

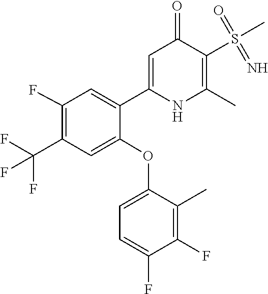 [SMILES: Cc1[nH]c(-c2cc(F)c(C(F)(F)F)cc2Oc2ccc(F)c(F)c2C)cc(=O)c1S(C)(=N)=O]

6-[2-(3,4-difluoro-2-methyl-phenoxy)-5-fluoro-4-
(trifluoromethyl)phenyl]-2-methyl-3-
(methylsulfonimidoyl)-1H-pyridin-4-one

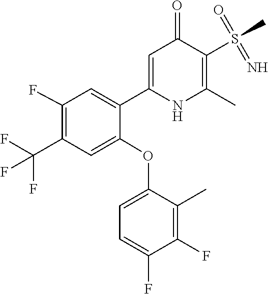 [SMILES: Cc1[nH]c(-c2cc(F)c(C(F)(F)F)cc2Oc2ccc(F)c(F)c2C)cc(=O)c1[S@@](C)(=N)=O]

(S)-6-[2-(3,4-difluoro-2-methyl-phenoxy)-5-
fluoro-4-(trifluoromethyl)phenyl]-2-methyl-3-
(methylsulfonimidoyl)-1H-pyridin-4-one

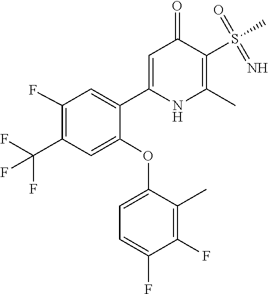 [SMILES: Cc1[nH]c(-c2cc(F)c(C(F)(F)F)cc2Oc2ccc(F)c(F)c2C)cc(=O)c1[S@](C)(=N)=O]

(R)-6-[2-(3,4-difluoro-2-methyl-phenoxy)-5-
fluoro-4-(trifluoromethyl)phenyl]-2-methyl-3-
(methylsulfonimidoyl)-1H-pyridin-4-one

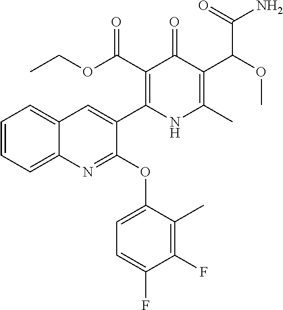 [SMILES: CCOC(=O)c1c(-c2cc3ccccc3nc2Oc2ccc(F)c(F)c2C)[nH]c(C)c(C(OC)C(N)=O)c1=O]

ethyl 5-(2-amino-1-methoxy-2-oxo-ethyl)-2-[2-
(3,4-difluoro-2-methyl-phenoxy)-3-quinolyl]-6-
methyl-4-oxo-1H-pyridine-3-carboxylate

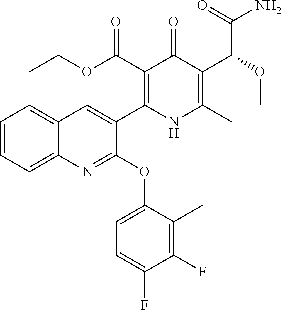 [SMILES: CCOC(=O)c1c(-c2cc3ccccc3nc2Oc2ccc(F)c(F)c2C)[nH]c(C)c([C@@H](OC)C(N)=O)c1=O]

ethyl (R)-5-(2-amino-1-methoxy-2-oxo-ethyl)-2-
[2-(3,4-difluoro-2-methyl-phenoxy)-3-quinolyl]-
6-methyl-4-oxo-1H-pyridine-3-carboxylate

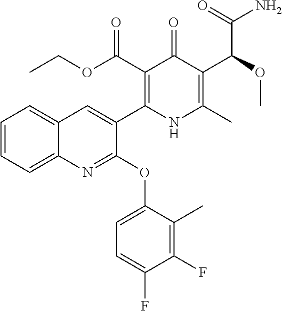 [SMILES: CCOC(=O)c1c(-c2cc3ccccc3nc2Oc2ccc(F)c(F)c2C)[nH]c(C)c([C@H](OC)C(N)=O)c1=O]

ethyl (S)-5-(2-amino-1-methoxy-2-oxo-ethyl)-2-
[2-(3,4-difluoro-2-methyl-phenoxy)-3-quinolyl]-
6-methyl-4-oxo-1H-pyridine-3-carboxylate

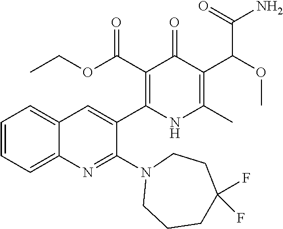 [SMILES: CCOC(=O)c1c(-c2cc3ccccc3nc2N2CCCC(F)(F)CC2)[nH]c(C)c(C(OC)C(N)=O)c1=O]

ethyl 5-(2-amino-1-methoxy-2-oxo-ethyl)-2-[2-
(4,4-difluoroazepan-1-yl)-3-quinolyl]-6-methyl-4-
oxo-1H-pyridine-3-carboxylate

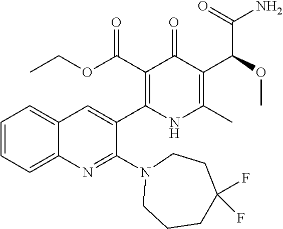 [SMILES: CCOC(=O)c1c(-c2cc3ccccc3nc2N2CCCC(F)(F)CC2)[nH]c(C)c([C@H](OC)C(N)=O)c1=O]

ethyl (S)-5-(2-amio-1-methoxy-2-oxo-ethyl)-2-
[2-(4,4-difluoroazepan-1-yl)-3-quinolyl]-6-
methyl-4-oxo-1H-pyridine-3-carboxylate

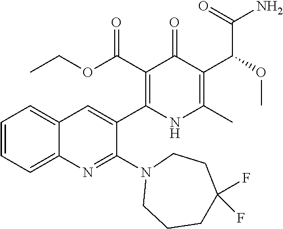 [SMILES: CCOC(=O)c1c(-c2cc3ccccc3nc2N2CCCC(F)(F)CC2)[nH]c(C)c([C@@H](OC)C(N)=O)c1=O]

ethyl (R)-5-(2-amino-methoxy-2-oxo-ethyl)-2-
[2-(4,4-difluoroazepan-1-yl)-3-quinolyl]-6-
methyl-4-oxo-1H-pyridine-3-carboxylate

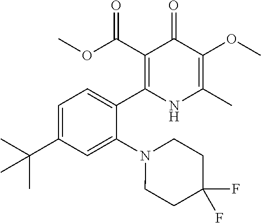 [SMILES: COC(=O)c1c(-c2ccc(C(C)(C)C)cc2N2CCC(F)(F)CC2)[nH]c(C)c(OC)c1=O]

methyl 2-[4-tert-butyl-2-(4,4-
difluorocyclohexyl)phenyl]-5-methoxy-6-methyl-
4-oxo-1H-pyridine-3-carboxylate

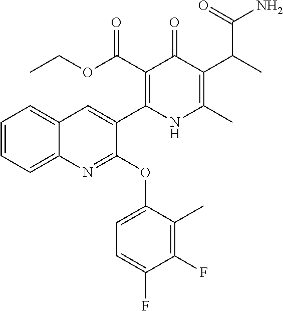 [SMILES: CCOC(=O)c1c(-c2cc3ccccc3nc2Oc2ccc(F)c(F)c2C)[nH]c(C)c(C(C)C(N)=O)c1=O]

ethyl 5-(2-amino-1-methyl-2-oxo-ethyl)-2-[2-
(3,4-difluoro-2-methyl-phenoxy)-3-quinolyl]-6-
methyl-4-oxo-1H-pyridine-3-carboxylate

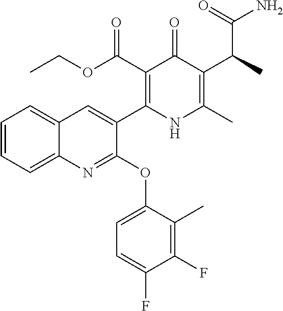 [SMILES: CCOC(=O)c1c(-c2cc3ccccc3nc2Oc2ccc(F)c(F)c2C)[nH]c(C)c([C@H](C)C(N)=O)c1=O]

ethyl (S)-5-(2-amino-1-methyl-2-oxo-ethyl)-2-[2-
(3,4-difluoro-2-methyl-phenoxy)-3-quinolyl]-6-
methyl-4-oxo-1H-pyridine-3-carboxylate

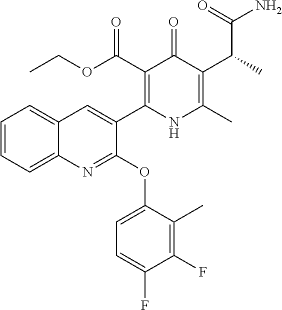 [SMILES: CCOC(=O)c1c(-c2cc3ccccc3nc2Oc2ccc(F)c(F)c2C)[nH]c(C)c([C@@H](C)C(N)=O)c1=O]

ethyl (R)-5-(2-amino-1-methyl-2-oxo-ethyl)-2-[2-
(3,4-difluoro-2-methyl-phenoxy)-3-quinolyl]-6-
methyl-4-oxo-1H-pyridine-3-carboxylate

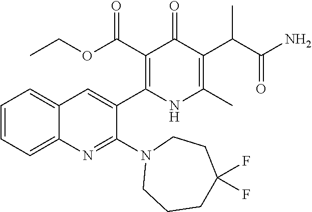 [SMILES: CCOC(=O)c1c(-c2cc3ccccc3nc2N2CCCC(F)(F)CC2)[nH]c(C)c(C(C)C(N)=O)c1=O]

ethyl 5-(2-amino-1-methyl-2-oxo-ethyl)-2-[2-
(4,4-difluoroazepan-1-yl)-3-quinolyl]-6-methyl-4-
oxo-1H-pyridine-3-carboxylate

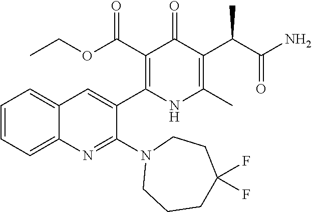 [SMILES: CCOC(=O)c1c(-c2cc3ccccc3nc2N2CCCC(F)(F)CC2)[nH]c(C)c([C@@H](C)C(N)=O)c1=O]

ethyl (R)-5-(2-amino-1-methyl-2-oxo-ethyl)-2-[2-
(4,4-difluoroazepan-1-yl)-3-quinolyl]-6-methyl-4-
oxo-1H-pyridine-3-carboxylate

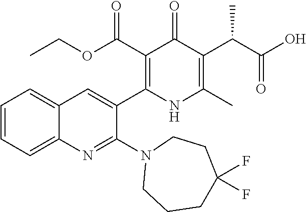 [SMILES: CCOC(=O)c1c(-c2cc3ccccc3nc2N2CCCC(F)(F)CC2)[nH]c(C)c([C@H](C)C(=O)O)c1=O]

ethyl (S)-5-(2-amino-1-methyl-2-oxo-ethyl)-2-[2-
(4,4-difluoroazepan-1-yl)-3-quinolyl]-6-methyl-4-
oxo-1H-pyridine-3-carboxylate

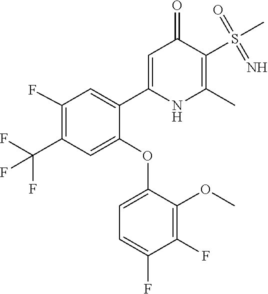 [SMILES: COc1c(Oc2cc(C(F)(F)F)c(F)cc2-c2cc(=O)c(S(C)(=N)=O)c(C)[nH]2)ccc(F)c1F]

6-[2-(3,4-difluoro-2-methoxy-phenoxy)-5-fluoro-
4-(trifluoromethyl)phenyl]-2-methyl-3-
(methylsulfonimidoyl)-1H-pyridin-4-one

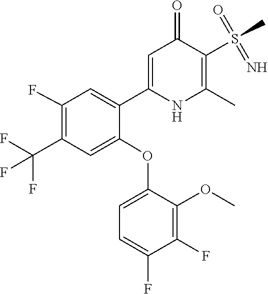 [SMILES: COc1c(Oc2cc(C(F)(F)F)c(F)cc2-c2cc(=O)c([S@@](C)(=N)=O)c(C)[nH]2)ccc(F)c1F]

(S)-6-[2-(3,4-difluoro-2-methoxy-phenoxy)-5-
fluoro-4-(trifluoromethyl)phenyl]-2-methyl-3-
(methylsulfonimidoyl)-1H-pyridin-4-one

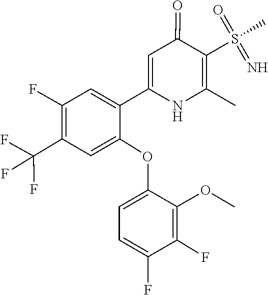 [SMILES: COc1c(Oc2cc(C(F)(F)F)c(F)cc2-c2cc(=O)c([S@](C)(=N)=O)c(C)[nH]2)ccc(F)c1F]

(R)-6-[2-(3,4-difluoro-2-methoxy-phenoxy)-5-
fluoro-4-(trifluoromethyl)phenyl]-2-methyl-3-
(methylsulfonimidoyl)-1H-pyridin-4-one

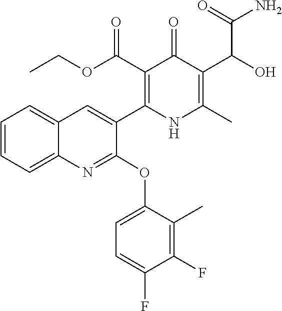 [SMILES: CCOC(=O)c1c(-c2cc3ccccc3nc2Oc2ccc(F)c(F)c2C)[nH]c(C)c(C(O)C(N)=O)c1=O]

ethyl 5-(2-amino-1-hydroxy-2-oxo-ethyl)-2-[2-
(3,4-difluoro-2-methyl-phenoxy)-3-quinolyl]-6-
methyl-4-oxo-1H-pyridine-3-carboxylate

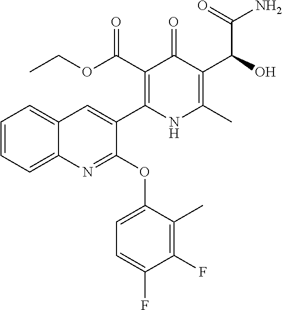 [SMILES: CCOC(=O)c1c(-c2cc3ccccc3nc2Oc2ccc(F)c(F)c2C)[nH]c(C)c([C@H](O)C(N)=O)c1=O]

ethyl (S)-5-(2-amino-1-hydroxy-2-oxo-ethyl)-2-
[2-(3,4-difluoro-2-methyl-phenoxy)-3-quinolyl]-
6-methyl-4-oxo-1H-pyridine-3-carboxylate

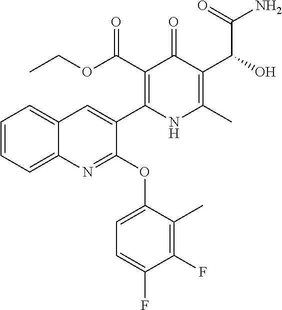 [SMILES: CCOC(=O)c1c(-c2cc3ccccc3nc2Oc2ccc(F)c(F)c2C)[nH]c(C)c([C@@H](O)C(N)=O)c1=O]

ethyl (R)-5-(2-amino-1-hydroxy-2-oxo-ethyl)-2-
[2-(3,4-difluoro-2-methyl-phenoxy)-3-quinolyl]-
6-methyl-4-oxo-1H-pyridine-3-carboxylate

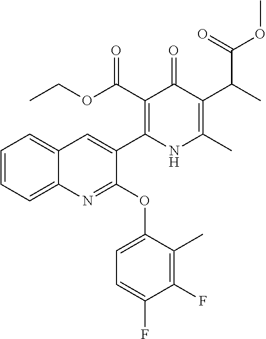 [SMILES: CCOC(=O)c1c(-c2cc3ccccc3nc2Oc2ccc(F)c(F)c2C)[nH]c(C)c(C(C)C(=O)OC)c1=O]

ethyl 2-[2-(3,4-difluoro-2-methyl-phenoxy)-3-
quinolyl]-5-(2-methoxy-1-methyl-2-oxo-ethyl)-6-
methyl-4-oxo-1H-pyridine-3-carboxylate

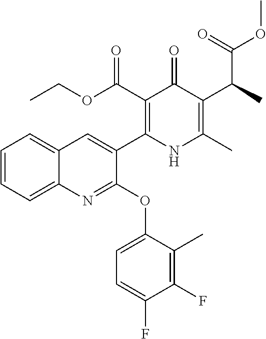 [SMILES: CCOC(=O)c1c(-c2cc3ccccc3nc2Oc2ccc(F)c(F)c2C)[nH]c(C)c([C@H](C)C(=O)OC)c1=O]

ethyl (S)-2-[2-(3,4-difluoro-2-methyl-phenoxy)-3-
quinolyl]-5-(2-methoxy-1-methyl-2-oxo-ethyl)-6-
methyl-4-oxo-1H-pyridine-3-carboxylate

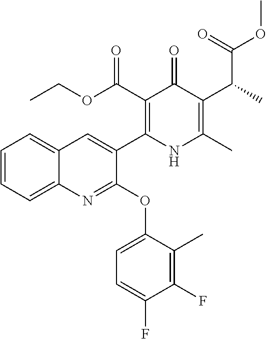 [SMILES: CCOC(=O)c1c(-c2cc3ccccc3nc2Oc2ccc(F)c(F)c2C)[nH]c(C)c([C@@H](C)C(=O)OC)c1=O]

ethyl (R)-2-[2-(3,4-difluoro-2-methyl-phenoxy)-3-
quinolyl]-5-(2-methoxy-1-methyl-2-oxo-ethyl)-6-
methyl-4-oxo-1H-pyridine-3-carboxylate

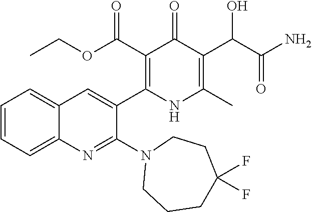 [SMILES: CCOC(=O)c1c(-c2cc3ccccc3nc2N2CCCC(F)(F)CC2)[nH]c(C)c(C(O)C(N)=O)c1=O]

ethyl 5-(2-amino-1-hydroxy-2-oxo-ethyl)-2-[2-
(4,4-difluoroazepan-1-yl)-3-quinolyl]-6-methyl-4-
oxo-1H-pyridine-3-carboxylate

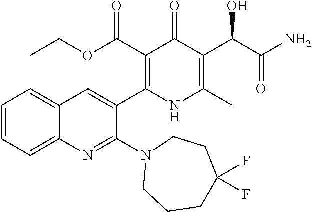 [SMILES: CCOC(=O)c1c(-c2cc3ccccc3nc2N2CCCC(F)(F)CC2)[nH]c(C)c([C@@H](O)C(N)=O)c1=O]

ethyl (R)-5-(2-amino-1-hydroxy-2-oxo-ethyl)-2-
[2-(4,4-difluoroazepan-1-yl)-3-quinolyl]-6-
methyl-4-oxo-1H-pyridine-3-carboxylate

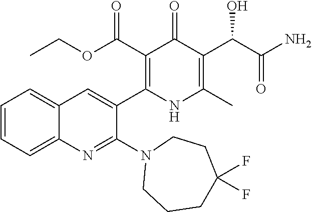 [SMILES: CCOC(=O)c1c(-c2cc3ccccc3nc2N2CCCC(F)(F)CC2)[nH]c(C)c([C@H](O)C(N)=O)c1=O]

ethyl (S)-5-(2-amino-1-hydroxy-2-oxo-ethyl)-2-
[2-(4,4-difluoroazepan-1-yl)-3-quinolyl]-6-
methyl-4-oxo-1H-pyridine-3-carboxylate

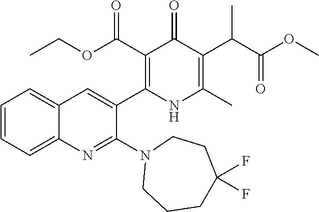 [SMILES: CCOC(=O)c1c(-c2cc3ccccc3nc2N2CCCC(F)(F)CC2)[nH]c(C)c(C(C)C(=O)OC)c1=O]

ethyl 2-[2-(4,4-difluoroazepan-1-yl)-3-quinolyl]-
5-(2-methoxy-1-methyl-2-oxo-ethyl)-6-methyl-4-
oxo-1H-pyridine-3-carboxylate

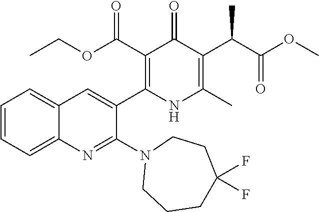 [SMILES: CCOC(=O)c1c(-c2cc3ccccc3nc2N2CCCC(F)(F)CC2)[nH]c(C)c([C@@H](C)C(=O)OC)c1=O]

ethyl (R)-2-[2-(4,4-difluoroazepan-1-yl)-3-
quinolyl]-5-(2-methoxy-1-methyl-2-oxo-ethyl)-6-
methyl-4-oxo-1H-pyridine-3-carboxylate

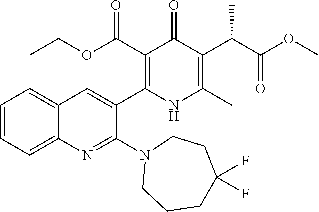 [SMILES: CCOC(=O)c1c(-c2cc3ccccc3nc2N2CCCC(F)(F)CC2)[nH]c(C)c([C@H](C)C(=O)OC)c1=O]

ethyl (S)-2-[2-(4,4-difluoroazepan-1-yl)-3-
quinolyl]-5-(2-methoxy-1-methyl-2-oxo-ethyl)-6-
methyl-4-oxo-1H-pyridine-3-carboxylate

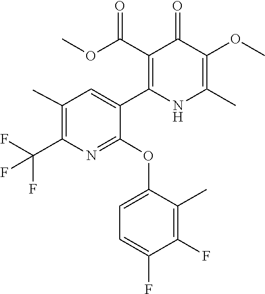 [SMILES: COC(=O)c1c(-c2cc(C)c(C(F)(F)F)nc2Oc2ccc(F)c(F)c2C)[nH]c(C)c(OC)c1=O]

methyl 2-[2-(3,4-difluoro-2-methyl-phenoxy)-5-
methyl-6-(trifluoromethyl)-3-pyridyl]-5-methoxy-
6-methyl-4-oxo-1H-pyridine-3-carboxylate

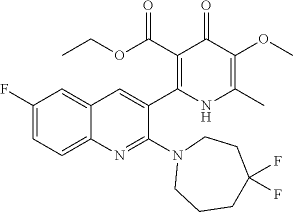 [SMILES: CCOC(=O)c1c(-c2cc3cc(F)ccc3nc2N2CCCC(F)(F)CC2)[nH]c(C)c(OC)c1=O]

ethyl 2-[2-(4,4-difluoroazepan-1-yl)-6-fluoro-3-
quinolyl]-5-methoxy-6-methyl-4-oxo-1H-
pyridine-3-carboxylate

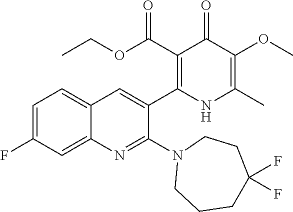 [SMILES: CCOC(=O)c1c(-c2cc3ccc(F)cc3nc2N2CCCC(F)(F)CC2)[nH]c(C)c(OC)c1=O]

ethyl 2-[2-(4,4-difluoroazepan-1-yl)-7-fluoro-3-
quinolyl]-5-methoxy-6-methyl-4-oxo-1H-
pyridine-3-carboxylate

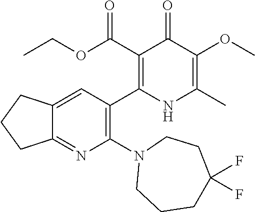 [SMILES: CCOC(=O)c1c(-c2cc3c(nc2N2CCCC(F)(F)CC2)CCC3)[nH]c(C)c(OC)c1=O]

ethyl 2-[2-(4,4-difluoroazepan-1-yl)-6,-dihydro-
5H-cyclopenta[b]pyridin-3-yl]-5-methoxy-6-
methyl-4-oxo-1H-pyridine-3-carboxylate

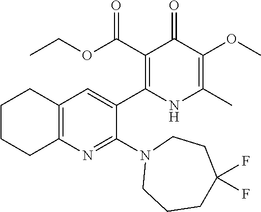 [SMILES: CCOC(=O)c1c(-c2cc3c(nc2N2CCCC(F)(F)CC2)CCCC3)[nH]c(C)c(OC)c1=O]

ethyl 2-[2-(4,4-difluoroazepan-1-yl)-5,6,7,8-
tetrahydroquinolin-3-yl]-5-methoxy-6-methyl-4-
oxo-1H-pyridine-3-carboxylate

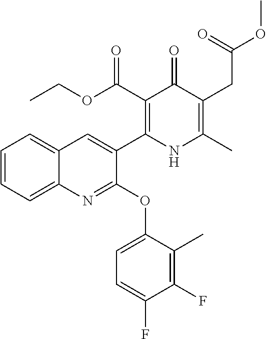 [SMILES: CCOC(=O)c1c(-c2cc3ccccc3nc2Oc2ccc(F)c(F)c2C)[nH]c(C)c(CC(=O)OC)c1=O]

ethyl 2-[2-(3,4-difluoro-2-methyl-phenoxy)-3-
quinolyl]-5-(2-methoxy-2-oxo-ethyl)-6-methyl-4-
oxo-1H-pyridine-3-carboxylate

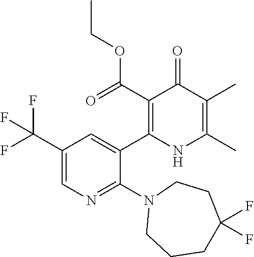 [SMILES: CCOC(=O)c1c(-c2cc(C(F)(F)F)cnc2N2CCCC(F)(F)CC2)[nH]c(C)c(C)c1=O]

ethyl 2-[2-(4,4-difluoroazepan-1-yl)-5-
(trifluoromethyl)-3-pyridyl]-5,6-dimethyl-4-oxo-
1H-pyridine-3-carboxylate

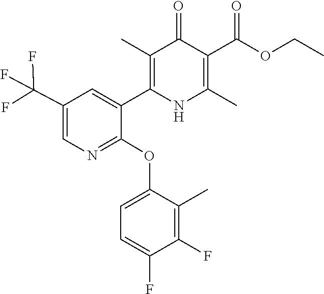 [SMILES: CCOC(=O)c1c(C)[nH]c(-c2cc(C(F)(F)F)cnc2Oc2ccc(F)c(F)c2C)c(C)c1=O]

ethyl 6-[2-(3,4-difluoro-2-methyl-phenoxy)-5-
(trifluoromethyl)-3-pyridyl]-2,5-dimethyl-4-oxo-
1H-pyridine-3-carboxylate

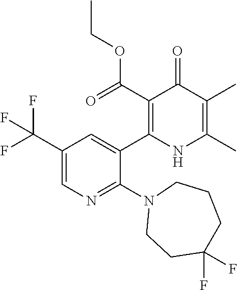 [SMILES: CCOC(=O)c1c(-c2cc(C(F)(F)F)cnc2N2CCCC(F)(F)CC2)[nH]c(C)c(C)c1=O]

ethyl 2-[5-(4,4-difluoroazepan-1-yl)-2-
(trifluoromethyl)-4-pyridyl]-5,6-dimethyl-4-oxo-
1H-pyridine-3-carboxylate

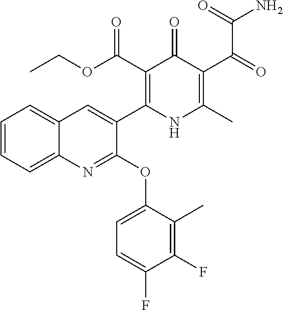 [SMILES: CCOC(=O)c1c(-c2cc3ccccc3nc2Oc2ccc(F)c(F)c2C)[nH]c(C)c(C(=O)C(N)=O)c1=O]

ethyl 2-[2-(3,4-difluoro-2-methyl-phenoxy)-3-
quinolyl]-6-methyl-5-oxamoyl-4-oxo-1H-
pyridine-3-carboxylate

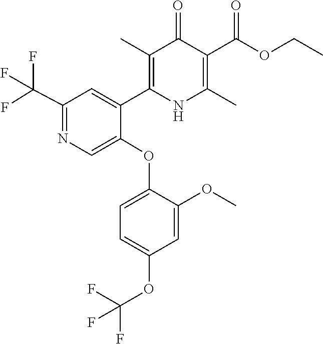 [SMILES: CCOC(=O)c1c(C)[nH]c(-c2cc(C(F)(F)F)ncc2Oc2ccc(OC(F)(F)F)cc2OC)c(C)c1=O]

ethyl 6-[5-[2-methoxy-4-
(trifluoromethoxy)phenoxy]-2-(trifluoromethyl)-
4-pyridyl]-2,5-dimethyl-4-oxo-1H-pyridine-3-
carboxylate

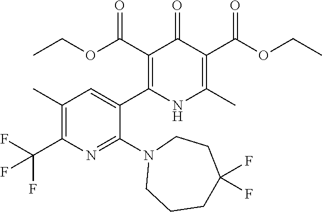 [SMILES: CCOC(=O)c1c(C)[nH]c(-c2cc(C)c(C(F)(F)F)nc2N2CCCC(F)(F)CC2)c(C(=O)OCC)c1=O]

diethyl 2-[2-(4,4-difluoroazepan-1-yl)-5-methyl-
6-(trifluoromethyl)-3-pyridyl]-6-methyl-4-oxo-
1H-pyridine-3,5-dicarboxylate

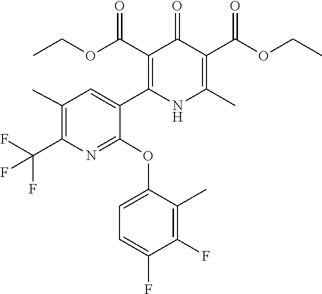 [SMILES: CCOC(=O)c1c(C)[nH]c(-c2cc(C)c(C(F)(F)F)nc2Oc2ccc(F)c(F)c2C)c(C(=O)OCC)c1=O]

diethyl 2-[2-(3,4-difluoro-2-methyl-phenoxy)-5-
methyl-6-(trifluoromethyl)-3-pyridyl]-6-methyl-4-
oxo-1H-pyridine-3,5-dicarboxylate

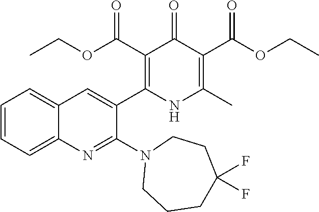 [SMILES: CCOC(=O)c1c(C)[nH]c(-c2cc3ccccc3nc2N2CCCC(F)(F)CC2)c(C(=O)OCC)c1=O]

diethyl 2-[2-(4,4-difluoroazepan-1-yl)-3-
quinolyl]-6-methyl-4-oxo-1H-pyridine-3,5-
dicarboxylate

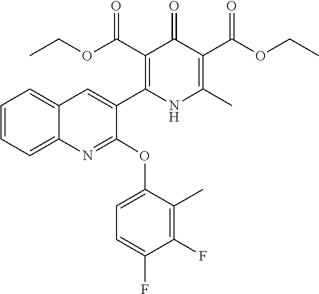 [SMILES: CCOC(=O)c1c(C)[nH]c(-c2cc3ccccc3nc2Oc2ccc(F)c(F)c2C)c(C(=O)OCC)c1=O]

diethyl 2-[2-(3,4-difluoro-2-methyl-phenoxy)-3-
quinolyl]-6-methyl-4-oxo-1H-pyridine-3,5-
dicarboxylate

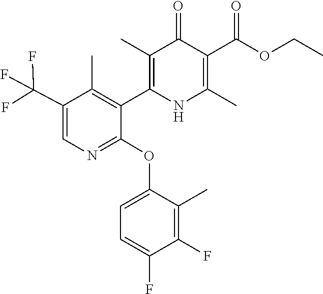 [SMILES: CCOC(=O)c1c(C)[nH]c(-c2c(Oc3ccc(F)c(F)c3C)ncc(C(F)(F)F)c2C)c(C)c1=O]

ethyl 6-[2-(3,4-difluoro-2-methyl-phenoxy)-4-
methyl-5-(trifluoromethyl)-3-pyridyl]-2,5-
dimethyl-4-oxo-1H-pyridine-3-carboxylate

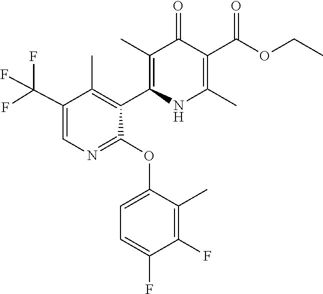

ethyl 6-[2-(3,4-difluoro-2-methyl-phenoxy)-4-
methyl-5-(trifluoromethyl)-3-pyridyl]-2,5-
dimethyl-4-oxo-1H-pyridine-3-carboxylate

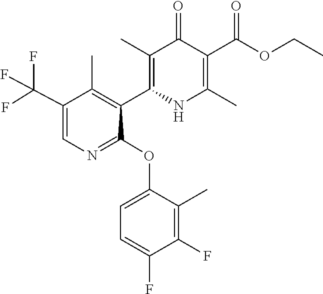

ethyl 6-[2-(3,4-difluoro-2-methyl-phenoxy)-4-
methyl-5-(trifluoromethyl)-3-pyridyl]-2,5-
dimethyl-4-oxo-1H-pyridine-3-carboxylate

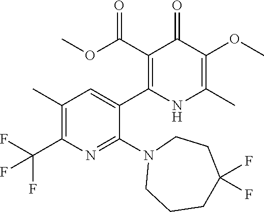 [SMILES: COC(=O)c1c(-c2cc(C)c(C(F)(F)F)nc2N2CCCC(F)(F)CC2)[nH]c(C)c(OC)c1=O]

methyl 2-[2-(4,4-difluoroazepan-1-yl)-5-methyl-
6-(trifluoromethyl)-3-pyridyl]-5-methoxy-6-
methyl-4-oxo-1H-pyridine-3-carboxylate

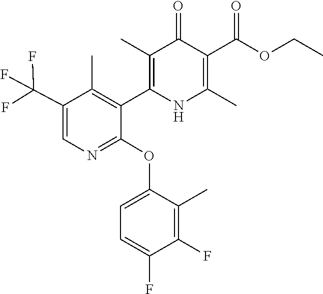 [SMILES: CCOC(=O)c1c(C)[nH]c(-c2c(Oc3ccc(F)c(F)c3C)ncc(C(F)(F)F)c2C)c(C)c1=O]

ethyl 6-[2-(3,4-difluoro-2-methyl-phenoxy)-4-
methyl-5-(trifluoromethyl)-3-pyridyl]-2,5-
dimethyl-4-oxo-1H-pyridine-3-carboxylate

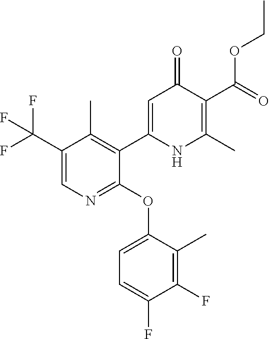 [SMILES: CCOC(=O)c1c(C)[nH]c(-c2c(Oc3ccc(F)c(F)c3C)ncc(C(F)(F)F)c2C)cc1=O]

ethyl 6-[2-(3,4-difluoro-2-methyl-phenoxy)-4-
methyl-5-(trifluoromethyl)-3-pyridyl]-2-methyl-4-
oxo-1H-pyridine-3-carboxylate

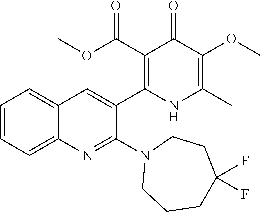 [SMILES: COC(=O)c1c(-c2cc3ccccc3nc2N2CCCC(F)(F)CC2)[nH]c(C)c(OC)c1=O]

methyl 2-[2-(4,4-difluoroazepan-1-yl)-3-
quinolyl]-5-methoxy-6-methyl-4-oxo-1H-
pyridine-3-carboxylate

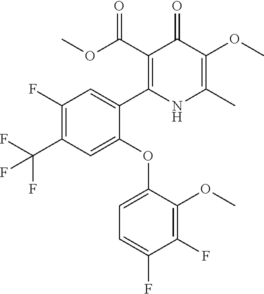 [SMILES: COC(=O)c1c(-c2cc(F)c(C(F)(F)F)cc2Oc2ccc(F)c(F)c2OC)[nH]c(C)c(OC)c1=O]

methyl 2-[2-(3,4-difluoro-2-methoxy-phenoxy)-5-
fluoro-4-(trifluoromethyl)phenyl]-5-methoxy-6-
methyl-4-oxo-1H-pyridine-3-carboxylate

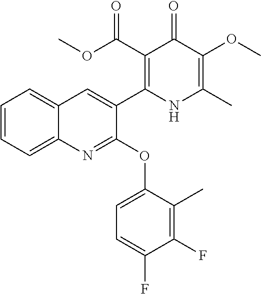 [SMILES: COC(=O)c1c(-c2cc3ccccc3nc2Oc2ccc(F)c(F)c2C)[nH]c(C)c(OC)c1=O]

methyl 2-[2-(3,4-difluoro-2-methyl-phenoxy)-3-
quinolyl]-5-methoxy-6-methyl-4-oxo-1H-
pyridine-3-carboxylate

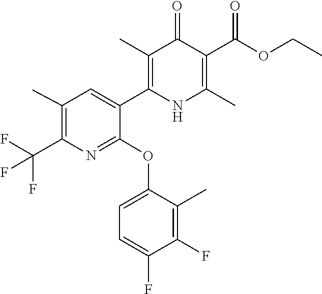 [SMILES: CCOC(=O)c1c(C)[nH]c(-c2cc(C)c(C(F)(F)F)nc2Oc2ccc(F)c(F)c2C)c(C)c1=O]

ethyl 6-[2-(3,4-difluoro-2-methyl-phenoxy)-5-
methyl-6-(trifluoromethyl)-3-pyridyl]-2,5-
dimethyl-4-oxo-1H-pyridine-3-carboxylate

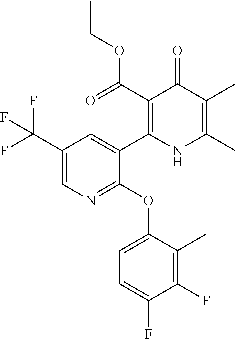 [SMILES: CCOC(=O)c1c(-c2cc(C(F)(F)F)cnc2Oc2ccc(F)c(F)c2C)[nH]c(C)c(C)c1=O]

ethyl 2-[2-(3,4-difluoro-2-methyl-phenoxy)-5-
(trifluoromethyl)-3-pyridyl]-5,6-dimethyl-4-oxo-
1H-pyridine-3-carboxylate

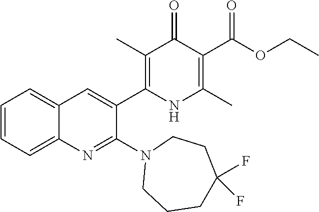 [SMILES: CCOC(=O)c1c(C)[nH]c(-c2cc3ccccc3nc2N2CCCC(F)(F)CC2)c(C)c1=O]

ethyl 6-[2-(4,4-difluoroazepan-1-yl)-3-quinolyl]-
2,5-dimethyl-4-oxo-1H-pyridine-3-carboxylate

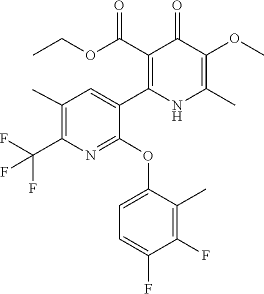 [SMILES: CCOC(=O)c1c(-c2cc(C)c(C(F)(F)F)nc2Oc2ccc(F)c(F)c2C)[nH]c(C)c(OC)c1=O]

ethyl 2-[2-(3,4-difluoro-2-methyl-phenoxy)-5-
methyl-6-(trifluoromethyl)-3-pyridyl]-5-methoxy-
6-methyl-4-oxo-1H-pyridine-3-carobxylate

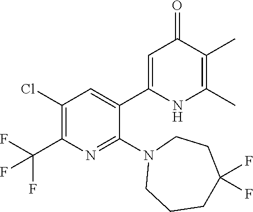 [SMILES: Cc1[nH]c(-c2cc(Cl)c(C(F)(F)F)nc2N2CCCC(F)(F)CC2)cc(=O)c1C]

6-[5-chloro-2-(4,4-difluoroazepan-1-yl)-6-
(trifluoromethyl)-3-pyridyl]-2,3-dimethyl-1H-
pyridin-4-one

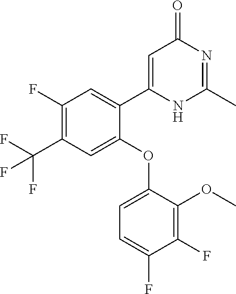 [SMILES: COc1c(Oc2cc(C(F)(F)F)c(F)cc2-c2cc(=O)nc(C)[nH]2)ccc(F)c1F]

6-[2-(3,4-difluoro-2-methoxy-phenoxy)-5-fluoro-
4-(trifluoromethyl)phenyl]-2-methyl-1H-
pyrimidin-4-one

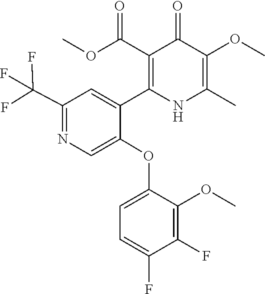 [SMILES: COC(=O)c1c(-c2cc(C(F)(F)F)ncc2Oc2ccc(F)c(F)c2OC)[nH]c(C)c(OC)c1=O]

methyl 2-[5-(3,4-difluoro-2-methoxy-phenoxy)-2-
(trifluoromethyl)-4-pyridyl]-5-methoxy-6-methyl-
4-oxo-1H-pyridine-3-carboxylate

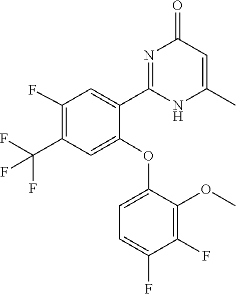 [SMILES: COc1c(Oc2cc(C(F)(F)F)c(F)cc2-c2nc(=O)cc(C)[nH]2)ccc(F)c1F]

2-[2-(3,4-difluoro-2-methoxy-phenoxy)-5-fluoro-
4-(trifluoromethyl)phenyl]-6-methyl-1H-
pyrimidin-4-one

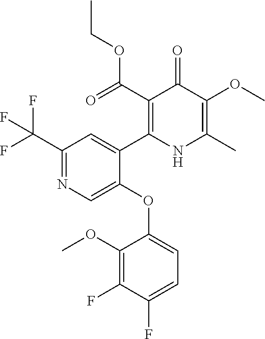 [SMILES: CCOC(=O)c1c(-c2cc(C(F)(F)F)ncc2Oc2ccc(F)c(F)c2OC)[nH]c(C)c(OC)c1=O]

ethyl 2-[5-(3,4-difluoro-2-methoxy-phenoxy)-2-
(trifluoromethyl)-4-pyridyl]-5-methoxy-6-methyl-
4-oxo-1H-pyridine-3-carboxylate

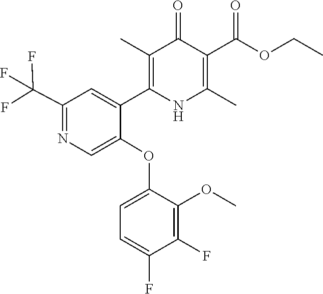 [SMILES: CCOC(=O)c1c(C)[nH]c(-c2cc(C(F)(F)F)ncc2Oc2ccc(F)c(F)c2OC)c(C)c1=O]

ethyl 6-[5-(3,4-difluoro-2-methoxy-phenoxy)-2-
(trifluoromethyl)-4-pyridyl]-2,5-dimethyl-4-oxo-
1H-pyridine-3-carboxylate

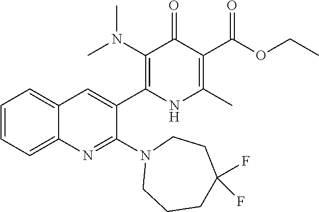 [SMILES: CCOC(=O)c1c(C)[nH]c(-c2cc3ccccc3nc2N2CCCC(F)(F)CC2)c(N(C)C)c1=O]

ethyl 6-[2-(4,4-difluoroazepan-1-yl)-3-quinolyl]-
5-(dimethylamino)-2-methyl-4-oxo-1H-pyridine-
3-carboxylate

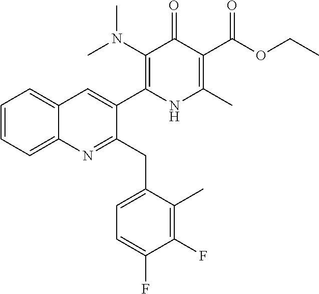 [SMILES: CCOC(=O)c1c(C)[nH]c(-c2cc3ccccc3nc2Cc2ccc(F)c(F)c2C)c(N(C)C)c1=O]

ethyl 6-[2-(3,4-difluoro-2-methyl-phenoxy)-3-
quinolyl]-5-(dimethylamino)-2-methyl-4-oxo-1H-
pyridine-3-carboxylate

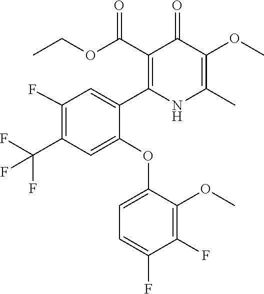 [SMILES: CCOC(=O)c1c(-c2cc(F)c(C(F)(F)F)cc2Oc2ccc(F)c(F)c2OC)[nH]c(C)c(OC)c1=O]

ethyl 2-[2-(3,4-difluoro-2-methoxy-phenoxy)-5-
fluoro-4-(trifluoromethyl)phenyl]-5-methoxy-6-
methyl-4-oxo-1H-pyridine-3-carboxylate

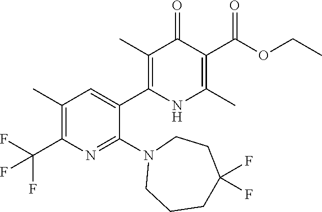 [SMILES: CCOC(=O)c1c(C)[nH]c(-c2cc(C)c(C(F)(F)F)nc2N2CCCC(F)(F)CC2)c(C)c1=O]

ethyl 6-[2-(4,4-difluoroazepan-1-yl)-5-methyl-6-
(trifluoromethyl)-3-pyridyl]-2,5-dimethyl-4-oxo-
1H-pyridine-3-carboxylate

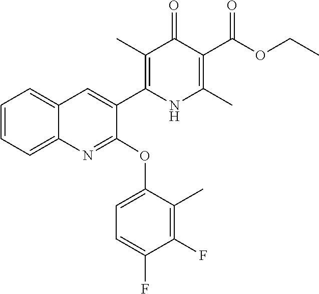 [SMILES: CCOC(=O)c1c(C)[nH]c(-c2cc3ccccc3nc2Oc2ccc(F)c(F)c2C)c(C)c1=O]

ethyl 6-[2-(3,4-difluoro-2-methyl-phenoxy)-3-
quinolyl]-2,5-dimethyl-4-oxo-1H-pyridine-3-
carboxylate

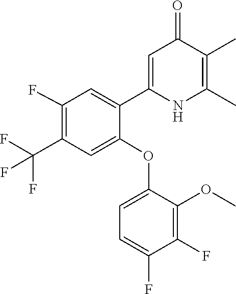 [SMILES: COc1c(Oc2cc(C(F)(F)F)c(F)cc2-c2cc(=O)c(C)c(C)[nH]2)ccc(F)c1F]

6-[2-(3,4-difluoro-2-methoxy-phenoxy)-5-fluoro-
4-(trifluoromethyl)phenyl]-2,3-dimethyl-1H-
pyridin-4-one

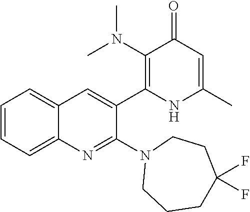 [SMILES: Cc1cc(=O)c(N(C)C)c(-c2cc3ccccc3nc2N2CCCC(F)(F)CC2)[nH]1]

2-[2-(4,4-difluoroazepan-1-yl)-3-quinolyl]-3-
(dimethylamino)-6-methyl-1H-pyridin-4-one

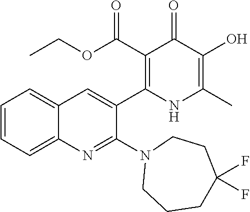 [SMILES: CCOC(=O)c1c(-c2cc3ccccc3nc2N2CCCC(F)(F)CC2)[nH]c(C)c(O)c1=O]

ethyl 2-[2-(4,4-difluoroazepan-1-yl)-3-quinolyl]-
5-hydroxy-6-methyl-4-oxo-1H-pyridine-3-
carboxylate

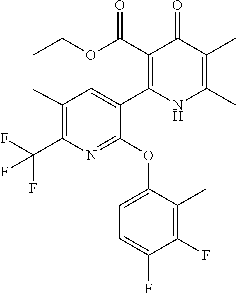 [SMILES: CCOC(=O)c1c(-c2cc(C)c(C(F)(F)F)nc2Oc2ccc(F)c(F)c2C)[nH]c(C)c(C)c1=O]

ethyl 2-[2-(3,4-difluoro-2-methyl-phenoxy)-5-
methyl-6-(trifluoromethyl)-3-pyridyl]-5,6-
dimethyl-4-oxo-1H-pyridine-3-carboxylate

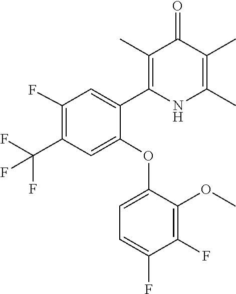 [SMILES: COc1c(Oc2cc(C(F)(F)F)c(F)cc2-c2[nH]c(C)c(C)c(=O)c2C)ccc(F)c1F]

2-[2-(3,4-difluoro-2-methoxy-phenoxy)-5-fluoro-
4-(trifluoromethyl)phenyl]-3,5,6-trimethyl-1H-
pyridin-4-one

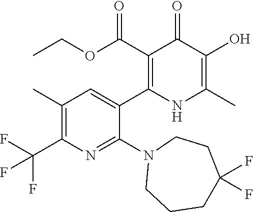 [SMILES: CCOC(=O)c1c(-c2cc(C)c(C(F)(F)F)nc2N2CCCC(F)(F)CC2)[nH]c(C)c(O)c1=O]

ethyl 2-[2-(4,4-difluoroazepan-1-yl)-5-methyl-6-
(trifluoromethyl)-3-pyridyl]-5-hydroxy-6-methyl-
4-oxo-1H-pyridine-3-carboxylate

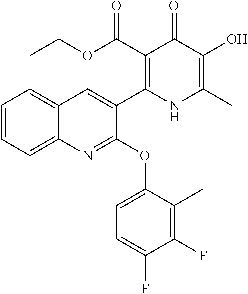 [SMILES: CCOC(=O)c1c(-c2cc3ccccc3nc2Oc2ccc(F)c(F)c2C)[nH]c(C)c(O)c1=O]

ethyl 2-[2-(3,4-difluoro-2-methyl-phenoxy)-3-
quinolyl]-5-hydroxy-6-methyl-4-oxo-1H-
pyridine-3-carboxylate

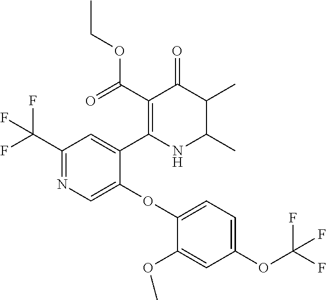 [SMILES: CCOC(=O)C1=C(c2cc(C(F)(F)F)ncc2Oc2ccc(OC(F)(F)F)cc2OC)NC(C)C(C)C1=O]

ethyl 2-[5-[2-methoxy-4-
(trifluoromethoxy)phenoxy]-2-(trifluoromethyl)-
4-pyridyl]-5,6-dimethyl-4-oxo-1H-pyridine-3-
carboxylate

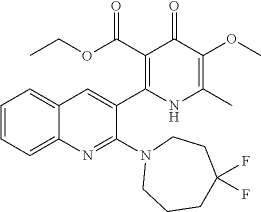 [SMILES: CCOC(=O)c1c(-c2cc3ccccc3nc2N2CCCC(F)(F)CC2)[nH]c(C)c(OC)c1=O]

ethyl 2-[2-(4,4-difluoroazepan-1-yl)-3-quinolyl]-
5-methoxy-6-methyl-4-oxo-1H-pyridin-3-
carboxylate

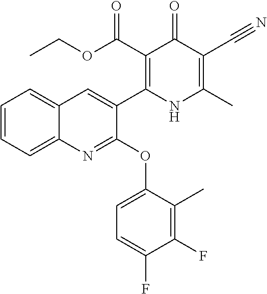 [SMILES: CCOC(=O)c1c(-c2cc3ccccc3nc2Oc2ccc(F)c(F)c2C)[nH]c(C)c(C#N)c1=O]

ethyl 5-cyano-2-[2-(3,4-difluoro-2-methyl-
phenoxy)-3-quinolyl]-6-methyl-4-oxo-1H-
pyridine-3-carboxylate

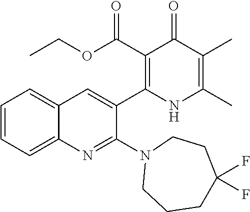 [SMILES: CCOC(=O)c1c(-c2cc3ccccc3nc2N2CCCC(F)(F)CC2)[nH]c(C)c(C)c1=O]

ethyl 2-[2-(4,4-difluoroazepan-1-yl)-3-quinolyl]-
5,6-dimethyl-4-oxo-1H-pyridine-3-carboxylate

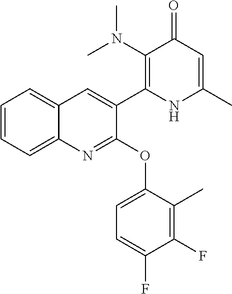 [SMILES: Cc1cc(=O)c(N(C)C)c(-c2cc3ccccc3nc2Oc2ccc(F)c(F)c2C)[nH]1]

2-[2-(3,4-difluoro-2-methyl-phenoxy)-3-
quinolyl]-3-(dimethylamino)-6-methyl-1H-
pyridin-4-one

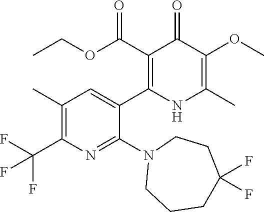 [SMILES: CCOC(=O)c1c(-c2cc(C)c(C(F)(F)F)nc2N2CCCC(F)(F)CC2)[nH]c(C)c(OC)c1=O]

ethyl 2-[2-(4,4-difluoroazepan-1-yl)-5-methyl-6-
(trifluoromethyl)-3-pyridyl]-5-methoxy-6-methyl-
4-oxo-1H-pyridine-3-carboxylate

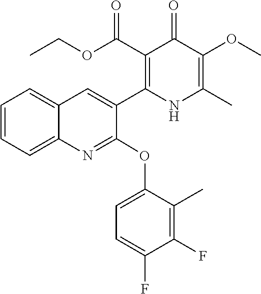 [SMILES: CCOC(=O)c1c(-c2cc3ccccc3nc2Oc2ccc(F)c(F)c2C)[nH]c(C)c(OC)c1=O]

ethyl 2-[2-(3,4-difluoro-2-methyl-phenoxy)-3-
quinolyl]-5-methoxy-6-methyl-4-oxo-1H-
pyridine-3-carboxylate

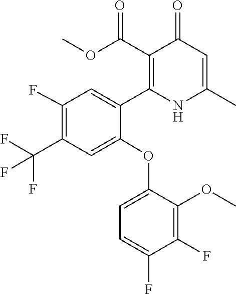 [SMILES: COC(=O)c1c(-c2cc(F)c(C(F)(F)F)cc2Oc2ccc(F)c(F)c2OC)[nH]c(C)cc1=O]

methyl 2-[2-(3,4-difluoro-2-methoxy-phenoxy)-5-
fluoro-4-(trifluoromethyl)phenyl]-6-mehtyl-4-
oxo-1H-pyridine-3-carboxylate

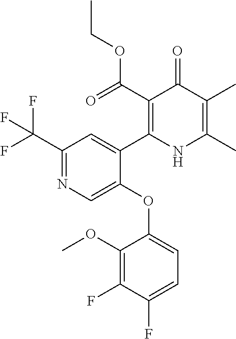 [SMILES: CCOC(=O)c1c(-c2cc(C(F)(F)F)ncc2Oc2ccc(F)c(F)c2OC)[nH]c(C)c(C)c1=O]

ethyl 2-[5-(3,4-difluoro-2-methoxy-phenoxy)-2-
(trifluoromethyl)-4-pyridiyl]-5,6-dimethyl-4-oxo-
1H-pyridine-3-carboxylate

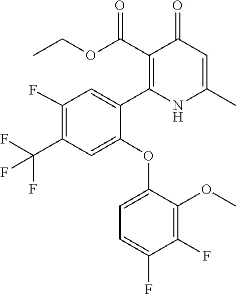 [SMILES: CCOC(=O)c1c(-c2cc(F)c(C(F)(F)F)cc2Oc2ccc(F)c(F)c2OC)[nH]c(C)cc1=O]

ethyl 2-[2-(3,4-difluoro-2-methoxy-phenoxy)-5-
fluoro-4-(trifluoromethyl)phenyl]-6-methyl-4-
oxo-1H-pyridine-3-carboxylate

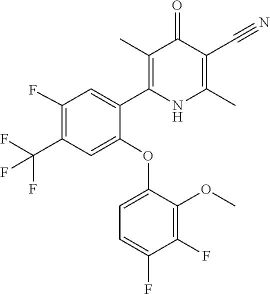 [SMILES: COc1c(Oc2cc(C(F)(F)F)c(F)cc2-c2[nH]c(C)c(C#N)c(=O)c2C)ccc(F)c1F]

6-[2-(3,4-difluoro-2-methoxy-phenoxy)-5-fluoro-
4-(trifluoromethyl)phenyl]-2,5-dimethyl-4-oxo-
1H-pyridine-3-carbonitrile

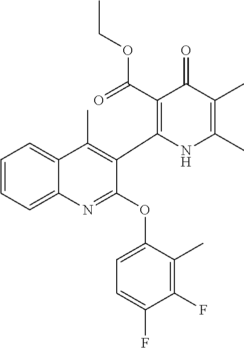 [SMILES: CCOC(=O)c1c(-c2c(Oc3ccc(F)c(F)c3C)nc3ccccc3c2C)[nH]c(C)c(C)c1=O]

ethyl 2-[2-(3,4-difluoro-2-methyl-phenoxy)-4-
methyl-3-quinolyl]-5,6-dimethyl-4-oxo-1H-
pyridine-3-carboxylate

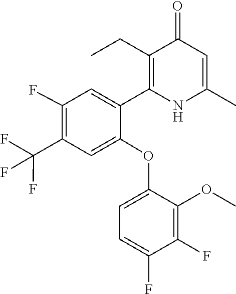 [SMILES: CCc1c(-c2cc(F)c(C(F)(F)F)cc2Oc2ccc(F)c(F)c2OC)[nH]c(C)cc1=O]

2-[2-(3,4-difluoro-2-methoxy-phenoxy)-5-fluoro-
4-(trifluoromethyl)phenyl]-3-methoxy-6-methyl-
1H-pyridin-4-one

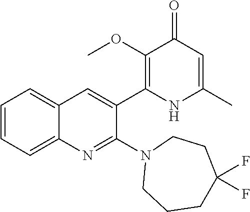 [SMILES: COc1c(-c2cc3ccccc3nc2N2CCCC(F)(F)CC2)[nH]c(C)cc1=O]

2-[2-(4,4-difluoroazepan-1-yl)-3-quinolyl]-3-
methoxy-6-methyl-1H-pyridin-4-one

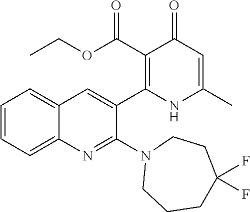 [SMILES: CCOC(=O)c1c(-c2cc3ccccc3nc2N2CCCC(F)(F)CC2)[nH]c(C)cc1=O]

ethyl 2-[2-(4,4-difluoroazepan-1-yl)-3-quinolyl]-
6-methyl-4-oxo-1H-pyridine-3-carboxylate

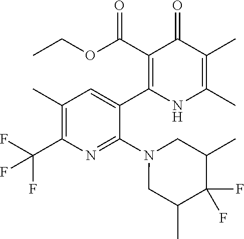 [SMILES: CCOC(=O)c1c(-c2cc(C)c(C(F)(F)F)nc2N2CC(C)C(F)(F)C(C)C2)[nH]c(C)c(C)c1=O]

ethyl 2-[2-[4,4-difluoro-3,5-dimethyl-1-
piperidyl]-5-methyl-6-(trifluoromethyl)-3-
pyridyl]-5,6-dimethyl-4-oxo-1H-pyridine-3-
carboxylate

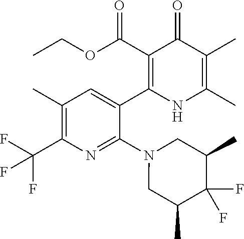 [SMILES: CCOC(=O)c1c(-c2cc(C)c(C(F)(F)F)nc2N2C[C@@H](C)C(F)(F)[C@@H](C)C2)[nH]c(C)c(C)c1=O]

ethyl 2-[2-[(3R,5S)-4,4-difluoro-3,5-dimethyl-1-
piperidyl]-5-methyl-6-(trifluoromethyl)-3-
pyridyl]-5,6-dimethyl-4-oxo-1H-pyridine-3-
carboxylate

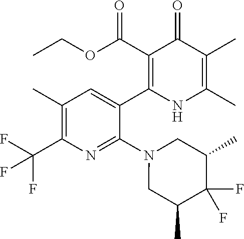 [SMILES: CCOC(=O)c1c(-c2cc(C)c(C(F)(F)F)nc2N2C[C@H](C)C(F)(F)[C@@H](C)C2)[nH]c(C)c(C)c1=O]

ethyl 2-[2-[(3S,5S)-4,4-difluoro-3,5-dimethyl-1-
piperidyl]-5-methyl-6-(trifluoromethyl)-3-
pyridyl]-5,6-dimethyl-4-oxo-1H-pyridin-3-
carboxylate

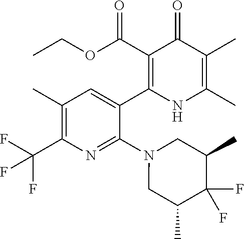 [SMILES: CCOC(=O)c1c(-c2cc(C)c(C(F)(F)F)nc2N2C[C@@H](C)C(F)(F)[C@H](C)C2)[nH]c(C)c(C)c1=O]

ethyl 2-[2-[(3R,5R)-4,4-difluoro-3,5-dimethyl-1-
piperidyl]-5-methyl-6-(trifluoromethyl)-3-
pyridyl]-5,6-dimethyl-4-oxo-1H-pyridine-3-
carboxylate

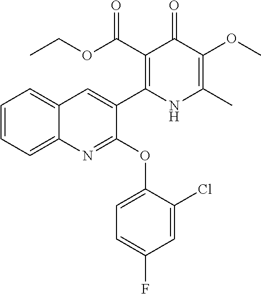 [SMILES: CCOC(=O)c1c(-c2cc3ccccc3nc2Oc2ccc(F)cc2Cl)[nH]c(C)c(OC)c1=O]

ethyl 2-[2-(2-chloro-4-fluoro-phenoxy)-3-
quinolyl]-5-methoxy-6-methyl-4-oxo-1H-
pyridine-3-carboxylate

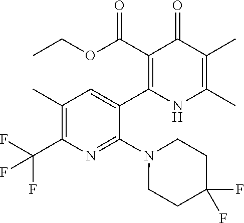 [SMILES: CCOC(=O)c1c(-c2cc(C)c(C(F)(F)F)nc2N2CCC(F)(F)CC2)[nH]c(C)c(C)c1=O]

ethyl 2-[2-(4,4-difluoro-1-piperidyl)-5-methyl-6-
(trifluoromethyl)-3-pyridyl]-5,6-dimethyl-4-oxo-
1H-pyridine-3-carboxylate

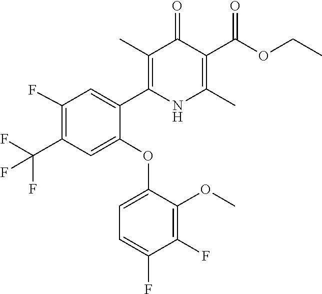 [SMILES: CCOC(=O)c1c(C)[nH]c(-c2cc(F)c(C(F)(F)F)cc2Oc2ccc(F)c(F)c2OC)c(C)c1=O]

ethyl 6-[2-(3,4-difluoro-2-methoxy-phenoxy)-5-
fluoro-4-(trifluoromethyl)phenyl]-2,5-dimethyl-4-
oxo-1H-pyridine-3-carboxylate

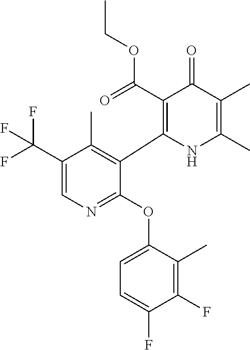 [SMILES: CCOC(=O)c1c(-c2c(Oc3ccc(F)c(F)c3C)ncc(C(F)(F)F)c2C)[nH]c(C)c(C)c1=O]

ethyl 2-[2-(3,4-difluoro-2-methyl-phenoxy)-4-
methyl-5-(trifluoromethyl)-3-pyridyl]-5,6-
dimethyl-4-oxo-1H-pyridine-3-carboxylate

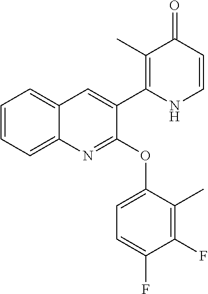 [SMILES: Cc1c(Oc2nc3ccccc3cc2-c2[nH]ccc(=O)c2C)ccc(F)c1F]

2-[2-(3,4-difluoro-2-methyl-phenoxy)-3-
quinolyl]-3-methyl-1H-pyridin-4-one

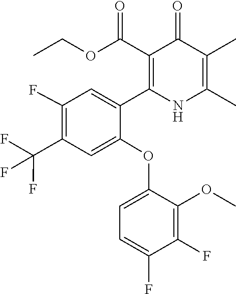 [SMILES: CCOC(=O)c1c(-c2cc(F)c(C(F)(F)F)cc2Oc2ccc(F)c(F)c2OC)[nH]c(C)c(C)c1=O]

ethyl 2-[2-(3,4-difluoro-2-methoxy-phenoxy)-5-
fluoro-4-(trifluoromethyl)phenyl]-5,6-dimethyl-4-
oxo-1H-pyridine-3-carboxylate

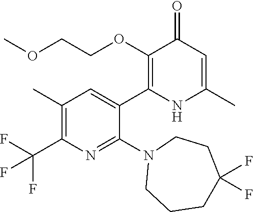 [SMILES: COCCOc1c(-c2cc(C)c(C(F)(F)F)nc2N2CCCC(F)(F)CC2)[nH]c(C)cc1=O]

2-[2-(4,4-difluoroazepan-1-yl)-5-methyl-6-
(trifluoromethyl)-3-pyridyl]-3-(2-
methoxyethoxy)-6-methyl-1H-pyridin-4-one

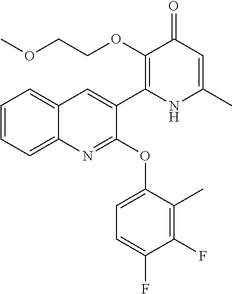 [SMILES: COCCOc1c(-c2cc3ccccc3nc2Oc2ccc(F)c(F)c2C)[nH]c(C)cc1=O]

2-[2-(3,4-difluoro-2-methyl-phenoxy)-3-
quinolyl]-3-(2-methoxyethoxy)-6-methyl-1H-
pyridin-4-one

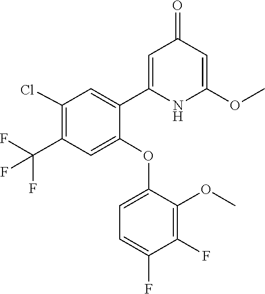 [SMILES: COc1cc(=O)cc(-c2cc(Cl)c(C(F)(F)F)cc2Oc2ccc(F)c(F)c2OC)[nH]1]

2-[5-chloro-2-(3,4-difluroo-2-methoxy-phenoxy)-
4-(trifluoromethyl)phenyl]-6-methoxy-1H-
pyridin-4-one

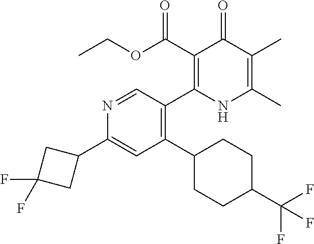 [SMILES: CCOC(=O)c1c(-c2cnc(C3CC(F)(F)C3)cc2C2CCC(C(F)(F)F)CC2)[nH]c(C)c(C)c1=O]

ethyl 2-[6-(3,3-difluorocyclobutyl)-4-[4-
(trifluoromethyl)cyclohexyl]-3-pyridyl]-5,6-
dimethyl-4-oxo-1H-pyridine-3-carboxylate

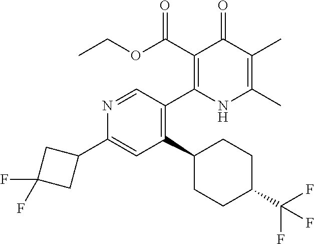 [SMILES: CCOC(=O)c1c(-c2cnc(C3CC(F)(F)C3)cc2[C@H]2CC[C@H](C(F)(F)F)CC2)[nH]c(C)c(C)c1=O]

ethyl 2-[6-(3,3-difluorocyclobutyl)-4-((1r,4r)4-
(trifluoromethyl)cyclohexyl)-3-pyridyl]-5,6-
dimethyl-4-oxo-1H-pyridine-3-carboxylate

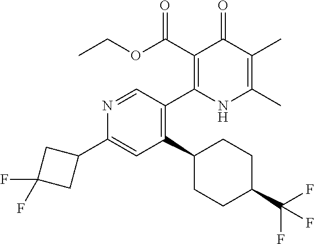 [SMILES: CCOC(=O)c1c(-c2cnc(C3CC(F)(F)C3)cc2[C@H]2CC[C@@H](C(F)(F)F)CC2)[nH]c(C)c(C)c1=O]

ethyl 2-[6-(3,3-difluorocyclobutyl)-4-((1s,4s)4-
(trifluoromethyl)cyclohexyl)-3-pyridyl]-5,6-
dimethyl-4-oxo-1H-pyridine-3-carboxylate

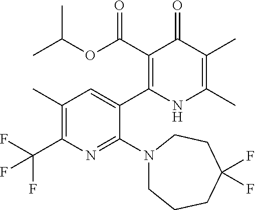 [SMILES: Cc1cc(-c2[nH]c(C)c(C)c(=O)c2C(=O)OC(C)C)c(N2CCCC(F)(F)CC2)nc1C(F)(F)F]

isopropyl 2-[2-(4,4-difluoroazepan-1-yl)-5-
methyl-6-(trifluoromethyl)-3-pyridyl]-5,6-
dimethyl-4-oxo-1H-pyridine-3-carboxylate

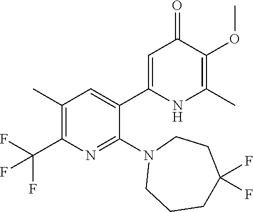 [SMILES: COc1c(C)[nH]c(-c2cc(C)c(C(F)(F)F)nc2N2CCCC(F)(F)CC2)cc1=O]

6-[2-(4,4-difluoroazepan-1-yl)-5-methyl-6-
(trifluoromethyl)-3-pyridyl]-3-methoxy-2-methyl-
1H-pyridin-4-one

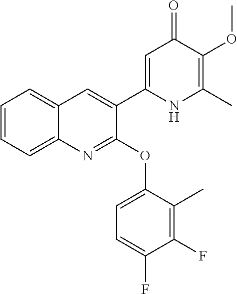 [SMILES: COc1c(C)[nH]c(-c2cc3ccccc3nc2Oc2ccc(F)c(F)c2C)cc1=O]

6-[2-(3,4-difluoro-2-methyl-phenxoy)-3-
quinolyl]-3-methoxy-2-methyl-1H-pyridin-4-one

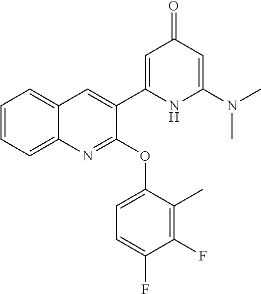 [SMILES: Cc1c(Oc2nc3ccccc3cc2-c2cc(=O)cc(N(C)C)[nH]2)ccc(F)c1F]

2-[2-(3,4-difluoro-2-methyl-phenoxy)-3-
quinolyl]-6-(dimethylamino)-1H-pyridin-4-one

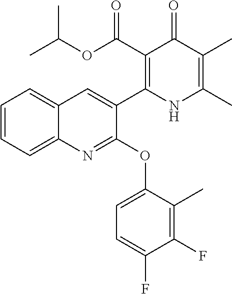 [SMILES: Cc1[nH]c(-c2cc3ccccc3nc2Oc2ccc(F)c(F)c2C)c(C(=O)OC(C)C)c(=O)c1C]

isopropyl 2-[2-(3,4-difluoro-2-methyl-phenoxy)-
3-quinolyl]-5,6-dimethyl-4-oxo-1H-pyridine-3-
carboxylate

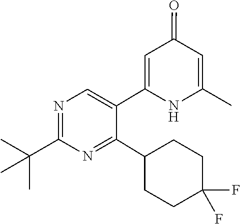 [SMILES: Cc1cc(=O)cc(-c2cnc(C(C)(C)C)nc2C2CCC(F)(F)CC2)[nH]1]

2-[2-tert-butyl-4-(4,4-
difluorocyclohexyl)pyrimidin-5-yl]-6-methyl-1H-
pyridin-4-one

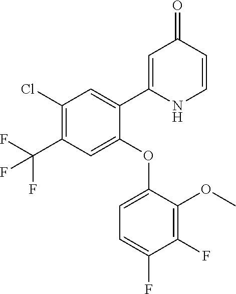 [SMILES: COc1c(Oc2cc(C(F)(F)F)c(Cl)cc2-c2cc(=O)cc[nH]2)ccc(F)c1F]

2-[5-chloro-2-(3,4-difluoro-2-methoxy-phenoxy)-
4-(trifluoromethyl)phenyl]-1H-pyridin-4-one

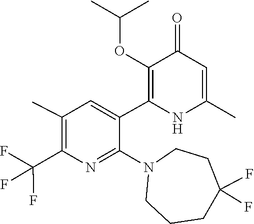 [SMILES: Cc1cc(=O)c(OC(C)C)c(-c2cc(C)c(C(F)(F)F)nc2N2CCCC(F)(F)CC2)[nH]1]

2-[2-(4,4-difluoroazepan-1-yl)-5-methyl-6-
(trifluoromethyl)-3-pyridyl]-3-isopropoxy-6-
methyl-1H-pyridin-4-one

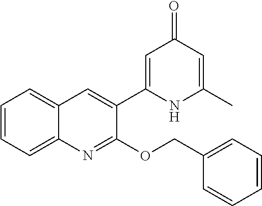 [SMILES: Cc1cc(=O)cc(-c2cc3ccccc3nc2OCc2ccccc2)[nH]1]

2-(2-benzyloxy-3-quinolyl)-6-methyl-1H-pyridin-
4-one

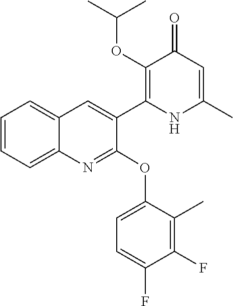 [SMILES: Cc1cc(=O)c(OC(C)C)c(-c2cc3ccccc3nc2Oc2ccc(F)c(F)c2C)[nH]1]

2-[2-(3,4-difluoro-2-methyl-phenoxy)-3-
quinolyl]-3-isopropoxy-6-methyl-1H-pyridin-4-
one

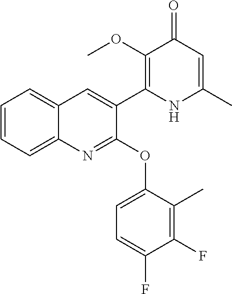 [SMILES: COc1c(-c2cc3ccccc3nc2Oc2ccc(F)c(F)c2C)[nH]c(C)cc1=O]

2-[2-(3,4-difluoro-2-methyl-phenoxy)-3-
quinolyl]-3-methoxy-6-methyl-1H-pyridin-4-one

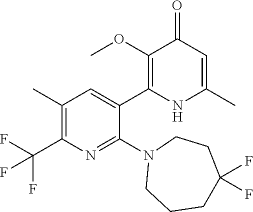 [SMILES: COc1c(-c2cc(C)c(C(F)(F)F)nc2N2CCCC(F)(F)CC2)[nH]c(C)cc1=O]

2-[2-(4,4-difluoroazepan-1-yl)-5-methyl-6-
(trifluoromethyl)-3-pyridyl]-3-methoxy-6-methyl-
1H-pyridin-4-one

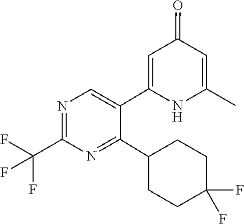 [SMILES: Cc1cc(=O)cc(-c2cnc(C(F)(F)F)nc2C2CCC(F)(F)CC2)[nH]1]

2-[4-(4,4-difluorocyclohexyl)-2-
(trifluoromethyl)pyrimidin-5-yl]-6-methyl-1H-
pyridin-4-one

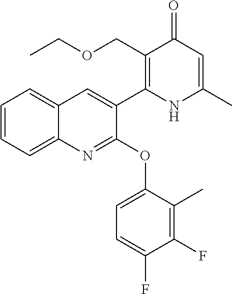 [SMILES: CCOCc1c(-c2cc3ccccc3nc2Oc2ccc(F)c(F)c2C)[nH]c(C)cc1=O]

2-[2-(3,4-difluoro-2-methyl-phenoxy)-3-
quinolyl]-3-(ethoxymethyl)-6-methyl-1H-pyridin-
4-one

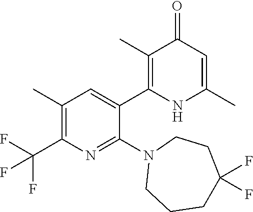 [SMILES: Cc1cc(=O)c(C)c(-c2cc(C)c(C(F)(F)F)nc2N2CCCC(F)(F)CC2)[nH]1]

2-[2-(4,4-difluoroazepan-1-yl)-5-methyl-6-
(trifluoromethyl)-3-pyridyl]-3,6-dimethyl-1H-
pyridin-4-one

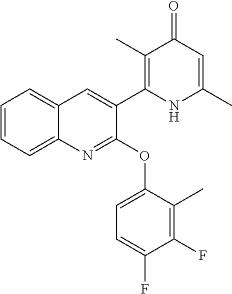 [SMILES: Cc1cc(=O)c(C)c(-c2cc3ccccc3nc2Oc2ccc(F)c(F)c2C)[nH]1]

2-[2-(3,4-difluoro-2-methyl-phenoxy)-3-
quinolyl]-3,6-dimethyl-1H-pyridin-4-one

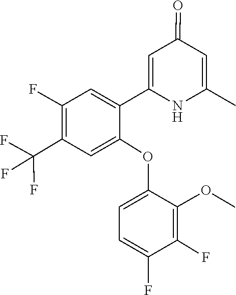 [SMILES: COc1c(Oc2cc(C(F)(F)F)c(F)cc2-c2cc(=O)cc(C)[nH]2)ccc(F)c1F]

2-[2-(3,4-difluoro-2-methoxy-phenoxy)-5-fluoro-
4-(trifluoromethyl)phenyl]-6-methyl-1H-pyridin-
4-one

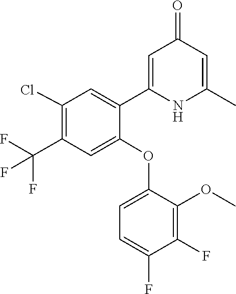 [SMILES: COc1c(Oc2cc(C(F)(F)F)c(Cl)cc2-c2cc(=O)cc(C)[nH]2)ccc(F)c1F]

2-[5-chloro-2-(3,4-difluoro-2-methoxy-phenoxy)-
4-(trifluoromethyl)phenyl]-6-methyl-1H-pyridin-
4-one

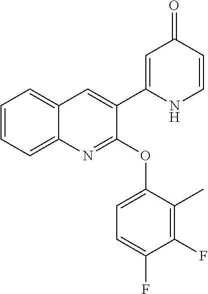 [SMILES: Cc1c(Oc2nc3ccccc3cc2-c2cc(=O)cc[nH]2)ccc(F)c1F]

2-[2-(3,4-difluoro-2-methyl-phenoxy)-3-
quinolyl]-1H-pyridin-4-one

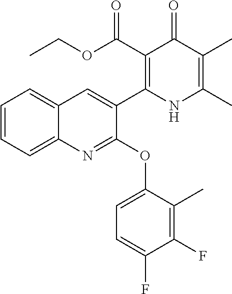 [SMILES: CCOC(=O)c1c(-c2cc3ccccc3nc2Oc2ccc(F)c(F)c2C)[nH]c(C)c(C)c1=O]

ethyl 2-[2-(3,4-difluoro-2-methyl-phenoxy)-3-
quinolyl]-5,6-dimethyl-4-oxo-1H-pyridine-3-
carboxylate

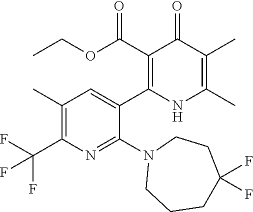 [SMILES: CCOC(=O)c1c(-c2cc(C)c(C(F)(F)F)nc2N2CCCC(F)(F)CC2)[nH]c(C)c(C)c1=O]

ethyl 2-[2-(4,4-difluoroazepan-1-yl)-5-methyl-6-
(trifluoromethyl)-3-pyridyl]-5,6-dimethyl-4-oxo-
1H-pyridine-3-carboxylate

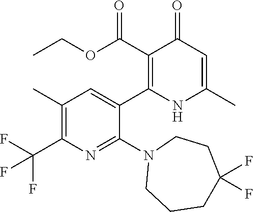 [SMILES: CCOC(=O)c1c(-c2cc(C)c(C(F)(F)F)nc2N2CCCC(F)(F)CC2)[nH]c(C)cc1=O]

ethyl 2-[2-(4,4-difluoroazepan-1-yl)-5-methyl-6-
(trifluoromethyl)-3-pyridyl]-6-methyl-4-oxo-1H-
pyridine-3-carboxylate

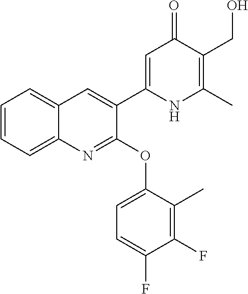 [SMILES: Cc1[nH]c(-c2cc3ccccc3nc2Oc2ccc(F)c(F)c2C)cc(=O)c1CO]

6-[2-(3,4-difluoro-2-methyl-phenoxy)-3-
quinolyl]-3-(hydroxymethyl)-2-methyl-1H-
pyridin-4-one

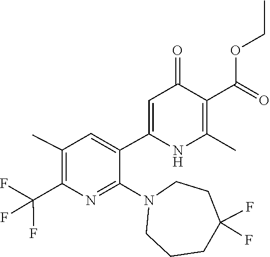 [SMILES: CCOC(=O)c1c(C)[nH]c(-c2cc(C)c(C(F)(F)F)nc2N2CCCC(F)(F)CC2)cc1=O]

ethyl 6-[2-(4,4-difluoroazepan-1-yl)-5-methyl-6-
(trifluoromethyl)-3-pyridyl]-2-methyl-4-oxo-1H-
pyridine-3-carboxylate

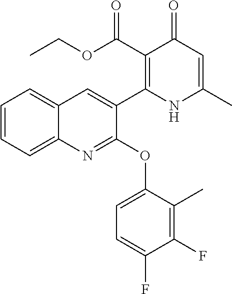 [SMILES: CCOC(=O)c1c(-c2cc3ccccc3nc2Oc2ccc(F)c(F)c2C)[nH]c(C)cc1=O]

ethyl 2-[2-(3,4-difluoro-2-methyl-phenoxy)-3-
quinolyl]-6-methyl-4-oxo-1H-pyridine-3-
carboxylate

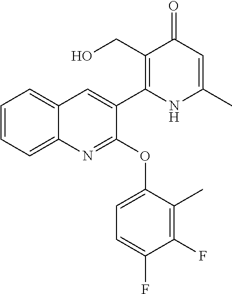 [SMILES: Cc1cc(=O)c(CO)c(-c2cc3ccccc3nc2Oc2ccc(F)c(F)c2C)[nH]1]

2-[2-(3,4-difluoro-2-methyl-phenoxy)-3-
quinolyl]-3-(hydroxymethyl)-6-methyl-1H-
pyridin-4-one

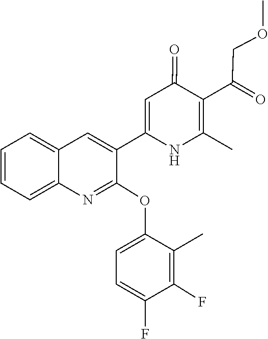 [SMILES: COCC(=O)c1c(C)[nH]c(-c2cc3ccccc3nc2Oc2ccc(F)c(F)c2C)cc1=O]

ethyl 6-[2-(3,4-difluoro-2-methyl-phenoxy)-3-
quinolyl]-2-methyl-4-oxo-1H-pyridine-3-
carboxylate

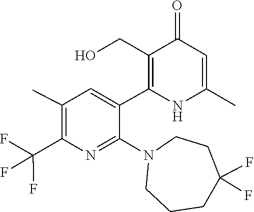 [SMILES: Cc1cc(=O)c(CO)c(-c2cc(C)c(C(F)(F)F)nc2N2CCCC(F)(F)CC2)[nH]1]

2-[2-(4,4-difluoroazepan-1-yl)-5-methyl-6-
(trifluoromethyl)-3-pyridyl]-3-(hydroxymethyl)-
6-methyl-1H-pyridin-4-one

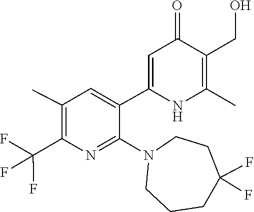 [SMILES: Cc1cc(-c2cc(=O)c(CO)c(C)[nH]2)c(N2CCCC(F)(F)CC2)nc1C(F)(F)F]

6-[2-(4,4-difluoroazepan-1-yl)-5-methyl-6-
(trifluoromethyl)-3-pyridyl]-3-(hydroxymethyl)-
2-methyl-1H-pyridin-4-one

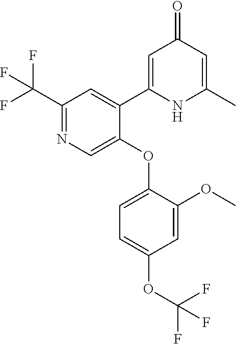 [SMILES: COc1cc(OC(F)(F)F)ccc1Oc1cnc(C(F)(F)F)cc1-c1cc(=O)cc(C)[nH]1]

2-[5-[2-methoxy-4-(trifluoromethoxy)phenoxy]-
2-(trifluoromethyl)-4-pyridyl]-6-methyl-1H-
pyridin-4-one

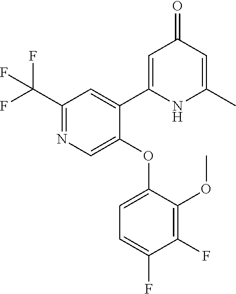 [SMILES: COc1c(Oc2cnc(C(F)(F)F)cc2-c2cc(=O)cc(C)[nH]2)ccc(F)c1F]

2-[5-(3,4-difluoro-2-methoxy-phenoxy)-2-
(trifluoromethyl)-4-pyridyl]-6-methyl-1H-pyridin-
4-one

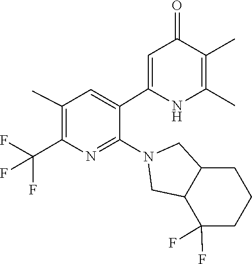 [SMILES: Cc1cc(-c2cc(=O)c(C)c(C)[nH]2)c(N2CC3CCCC(F)(F)C3C2)nc1C(F)(F)F]

6-[2-(7,7-difluoro-3,3a,4,5,6,7a-hexahydro-1H-
isoindol-2-yl)-5-methyl-6-(trifluoromethyl)-3-
pyridyl]-2,3-dimethyl-1H-pyridin-4-one

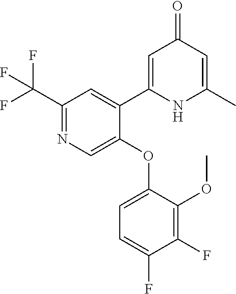 [SMILES: COc1c(Oc2cnc(C(F)(F)F)cc2-c2cc(=O)cc(C)[nH]2)ccc(F)c1F]

6-[2-(3,4-difluoro-2-methyl-phenoxy)-5-
(trifluoromethyl)-3-pyridyl]-2,3-dimethyl-1H-
pyridin-4-one

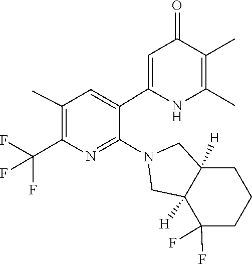 [SMILES: Cc1cc(-c2cc(=O)c(C)c(C)[nH]2)c(N2C[C@H]3CCCC(F)(F)[C@H]3C2)nc1C(F)(F)F]

6-[(3aR,7aS)-2-(7,7-difluoro-3,3a,,5,6,7a-
hexahydro-1H-isoindol-2-yl)-5-methyl-6-
(trifluoromethyl)-3-pyridyl]-2,3-dimethyl-1H-
pyridin-4-one

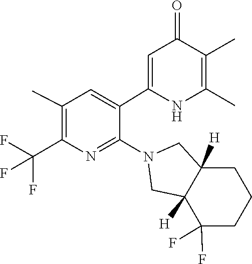 [SMILES: Cc1cc(-c2cc(=O)c(C)c(C)[nH]2)c(N2C[C@@H]3CCCC(F)(F)[C@@H]3C2)nc1C(F)(F)F]

6-[(3aS,7aR)-2-(7,7-difluoro-3,3a,4,5,6,7a-
hexahydro-1H-isoindol-2-yl)-5-methyl-6-
(trifluoromethyl)-3-pyridyl]-2,3-dimethyl-1H-
pyridin-4-one

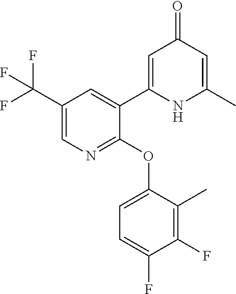 [SMILES: Cc1cc(=O)cc(-c2cc(C(F)(F)F)cnc2Oc2ccc(F)c(F)c2C)[nH]1]

2-[2-(3,4-difluoro-2-methyl-phenoxy)-5-
(trifluoromethyl)-3-pyridyl]-6-methyl-1H-pyridin-
4-one

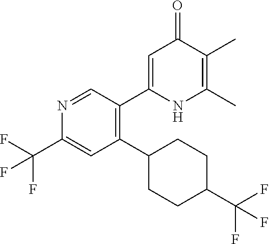 [SMILES: Cc1[nH]c(-c2cnc(C(F)(F)F)cc2C2CCC(C(F)(F)F)CC2)cc(=O)c1C]

2,3-dimethyl-6-[6-(trifluoromethyl)-4-[4-
(trifluoromethyl)cyclohexyl]-3-pyridyl]-1H-
pyridin-4-one

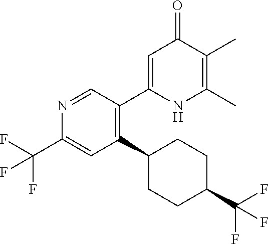 [SMILES: Cc1[nH]c(-c2cnc(C(F)(F)F)cc2[C@H]2CC[C@@H](C(F)(F)F)CC2)cc(=O)c1C]

2,3-dimethyl-6-[6-(trifluoromethyl)-4-((1s,4s)4-
(trifluoromethyl)cyclohexyl)-3-pyridyl]-1H-
pyridin-4-one

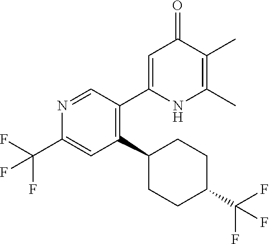 [SMILES: Cc1[nH]c(-c2cnc(C(F)(F)F)cc2[C@H]2CC[C@H](C(F)(F)F)CC2)cc(=O)c1C]

2,3-dimethyl-6-[6-(trifluoromethyl)-4-((1r,4r)4-
(trifluoromethyl)cyclohexyl)-3-pyridyl]-1H-
pyridin-4-one

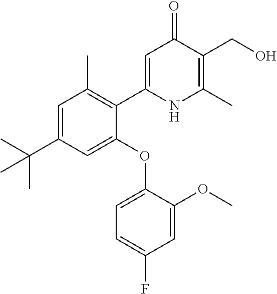 [SMILES: COc1cc(F)ccc1Oc1cc(C(C)(C)C)cc(C)c1-c1cc(=O)c(CO)c(C)[nH]1]

6-[4-tert-butyl-2-(4-fluoro-2-methoxy-phenoxy)-
6-methyl-phenyl]-3-(hydroxymethyl)-2-methyl-
1H-pyridin-4-one

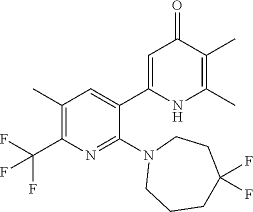 [SMILES: Cc1cc(-c2cc(=O)c(C)c(C)[nH]2)c(N2CCCC(F)(F)CC2)nc1C(F)(F)F]

6-[2-(4,4-difluoroazepan-1-yl)-5-methyl-6-
(trifluoromethyl)-3-pyridyl]-2,3-dimethyl-1H-
pyridin-4-one

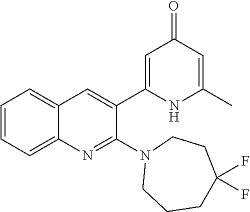 [SMILES: Cc1cc(=O)cc(-c2cc3ccccc3nc2N2CCCC(F)(F)CC2)[nH]1]

2-[2-(4,4-difluoroazepan-1-yl)-3-quinolyl]-6-
methyl-1H-pyridin-4-one

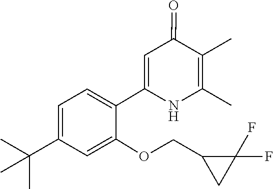 [SMILES: Cc1[nH]c(-c2ccc(C(C)(C)C)cc2OCC2CC2(F)F)cc(=O)c1C]

6-[4-tert-butyl-2-[(2,2-
difluorocyclopropyl)methoxy)phenyl]-2,3-
dimethyl-1H-pyridin-4-one

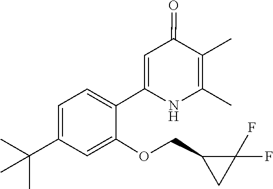 [SMILES: Cc1[nH]c(-c2ccc(C(C)(C)C)cc2OC[C@@H]2CC2(F)F)cc(=O)c1C]

(S)-6-[4-tert-butyl-2-[(2,2-
difluorocyclopropyl)methoxy]phenyl]-2,3-
dimethyl-1H-pyridin-4-one

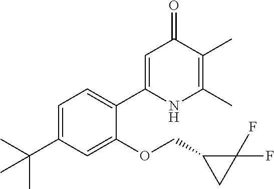 [SMILES: Cc1[nH]c(-c2ccc(C(C)(C)C)cc2OC[C@H]2CC2(F)F)cc(=O)c1C]

(R)-6-[4-tert-butyl-2-[(2,2-
difluorocyclopropyl)methoxy]phenyl]-2,3-
dimethyl-1H-pyridin-4-one

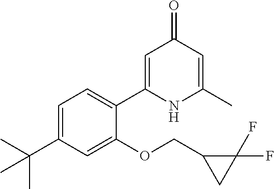 [SMILES: Cc1cc(=O)cc(-c2ccc(C(C)(C)C)cc2OCC2CC2(F)F)[nH]1]

2-[4-tert-butyl-2-[(2,2-
difluorocyclopropyl)methoxy]phenyl]-6-methyl-
1H-pyridin-4-one

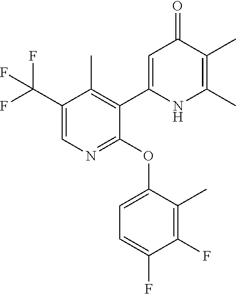 [SMILES: Cc1[nH]c(-c2c(Oc3ccc(F)c(F)c3C)ncc(C(F)(F)F)c2C)cc(=O)c1C]

6-[2-(3,4-difluoro-2-methyl-phenoxy)-4-methyl-
5-(trifluoromethyl)-3-pyridyl]-2,3-dimethyl-1H-
pyridin-4-one

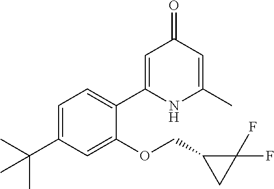 [SMILES: Cc1cc(=O)cc(-c2ccc(C(C)(C)C)cc2OC[C@H]2CC2(F)F)[nH]1]

(R)-2-[4-tert-butyl-2-[(2,2-
difluorocyclopropyl)methoxy]phenyl]-6-methyl-
1H-pyridin-4-one

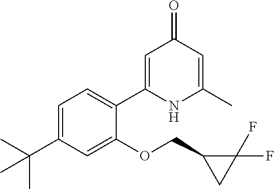 [SMILES: Cc1cc(=O)cc(-c2ccc(C(C)(C)C)cc2OC[C@@H]2CC2(F)F)[nH]1]

(S)-2-[4-tert-butyl-2-[(2,2-
difluorocyclopropyl)methoxy]phenyl]-6-methyl-
1H-pyridin-4-one

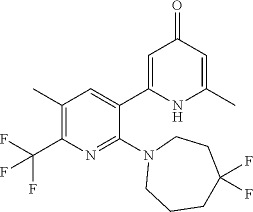 [SMILES: Cc1cc(=O)cc(-c2cc(C)c(C(F)(F)F)nc2N2CCCC(F)(F)CC2)[nH]1]

2-[2-(4,4-difluoroazepan-1-yl)-5-methyl-6-
(trifluoromethyl)-3-pyridyl]-6-methyl-1H-pyridin-
4-one

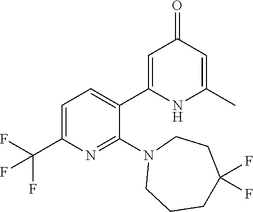 [SMILES: Cc1cc(=O)cc(-c2ccc(C(F)(F)F)nc2N2CCCC(F)(F)CC2)[nH]1]

2-[2-(4,4-difluoroazepan-1-yl)-6-
(trifluoromethyl)-3-pyridyl]-6-methyl-1H-pyridin-
4-one

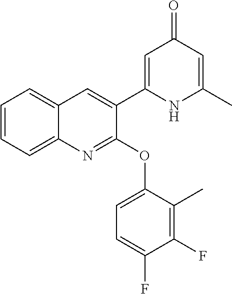 [SMILES: Cc1cc(=O)cc(-c2cc3ccccc3nc2Oc2ccc(F)c(F)c2C)[nH]1]

2-[2-(3,4-difluoro-2-methyl-phenoxy)-3-
quinolyl]-6-methyl-1H-pyridin-4-one

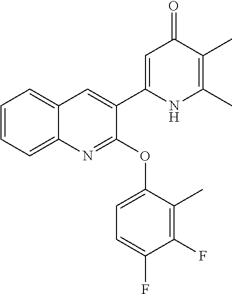 [SMILES: Cc1[nH]c(-c2cc3ccccc3nc2Oc2ccc(F)c(F)c2C)cc(=O)c1C]

6-[2-(3,4-difluoro-2-methyl-phenoxy)-3-
quinolyl]-2,3-dimethyl-1H-pyridin-4-one

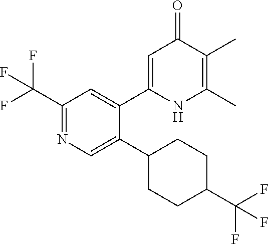 [SMILES: Cc1[nH]c(-c2cc(C(F)(F)F)ncc2C2CCC(C(F)(F)F)CC2)cc(=O)c1C]

2,3-dimethyl-6-[2-(trifluoromethyl)-5-[4-
(trifluoromethyl)cyclohexyl]-4-pyridyl]-1H_
pyridin-4-one

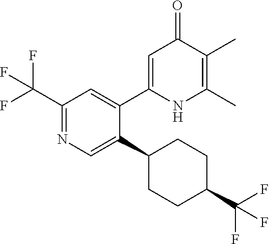 [SMILES: Cc1[nH]c(-c2cc(C(F)(F)F)ncc2[C@H]2CC[C@@H](C(F)(F)F)CC2)cc(=O)c1C]

2,3-dimethyl-6-[2-(trifluoromethyl)-5-((1s,4s)-4-
(trifluoromethyl)cyclohexyl)-4-pyridyl]-1H-
pyridin-4-one

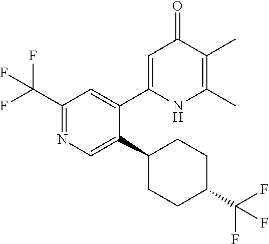 [SMILES: Cc1[nH]c(-c2cc(C(F)(F)F)ncc2[C@H]2CC[C@H](C(F)(F)F)CC2)cc(=O)c1C]

2,3-dimethyl-6-[2-(trifluoromethyl)-5-((1r,4r)-4-
(trifluoromethyl)cyclohexyl)-4-pyridyl]-1H-
pyridin-4-one

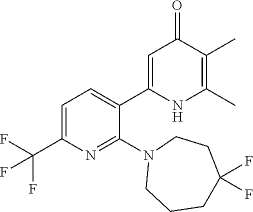 [SMILES: Cc1[nH]c(-c2ccc(C(F)(F)F)nc2N2CCCC(F)(F)CC2)cc(=O)c1C]

6-[2-(4,4-difluoroazepan-1-yl)-6-
(trifluoromethyl)-3-pyridyl]-2,3-dimethyl-1H-
pyridin-4-one

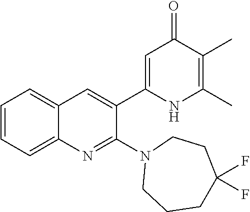 [SMILES: Cc1[nH]c(-c2cc3ccccc3nc2N2CCCC(F)(F)CC2)cc(=O)c1C]

6-[2-(4,4-difluoroazepan-1-yl)-3-quinolyl]-2,3-
dimethyl-1H-pyridin-4-one

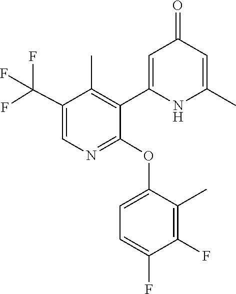 [SMILES: Cc1cc(=O)cc(-c2c(Oc3ccc(F)c(F)c3C)ncc(C(F)(F)F)c2C)[nH]1]

2-[2-(3,4-difluoro-2-methyl-phenoxy)-4-methyl-
5-(trifluoromethyl)-3-pyridyl]-6-methyl-1H-
pyridin-4-one

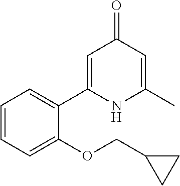 [SMILES: Cc1cc(=O)cc(-c2ccccc2OCC2CC2)[nH]1]

2-[2-(cyclopropylmethoxy)phenyl]-6-methyl-1H-
pyridin-4-one

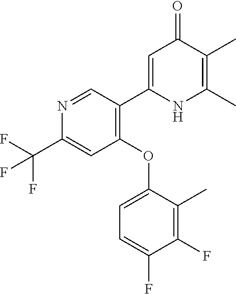 [SMILES: Cc1[nH]c(-c2cnc(C(F)(F)F)cc2Oc2ccc(F)c(F)c2C)cc(=O)c1C]

6-[4-(3,4-difluoro-2-methyl-phenoxy)-6-
(trifluoromethyl)-3-pyridyl]-2,3-dimethyl-1H-
pyridin-4-one

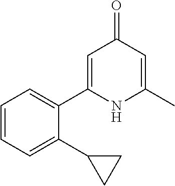 [SMILES: Cc1cc(=O)cc(-c2ccccc2C2CC2)[nH]1]

2-(2-cyclopropylphenyl)-6-methyl-1H-pyridin-4-
one

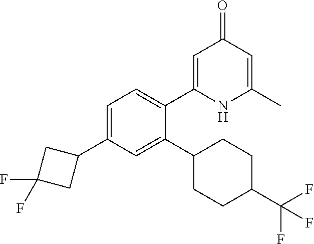 [SMILES: Cc1cc(=O)cc(-c2ccc(C3CC(F)(F)C3)cc2C2CCC(C(F)(F)F)CC2)[nH]1]

2-[4-(3,3-difluorocylcobutyl)-2-[4-
(trifluoromethyl)cyclohexyl]phenyl]-6-methyl-
1H-pyridin-4-one

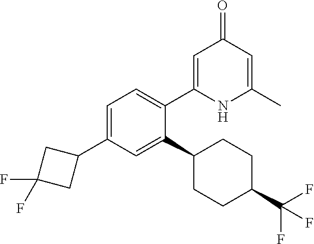 [SMILES: Cc1cc(=O)cc(-c2ccc(C3CC(F)(F)C3)cc2[C@H]2CC[C@@H](C(F)(F)F)CC2)[nH]1]

2-[4-(3,3-difluorocyclobutyl)-2-((1s,4s)-4-
(trifluoromethyl)cyclohexyl)phenyl]-6-methyl-
1H-pyridin-4-one

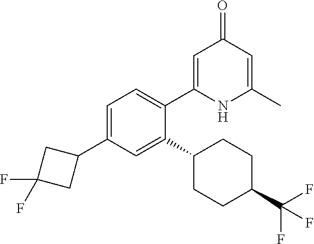 [SMILES: Cc1cc(=O)cc(-c2ccc(C3CC(F)(F)C3)cc2[C@H]2CC[C@H](C(F)(F)F)CC2)[nH]1]

2-[4-(3,3-difluorocyclobutyl)-2-((1r,4r)-4-
(trifluoromethyl)cyclohexyl]phenyl]-6-methyl-
1H-pyridin-4-one

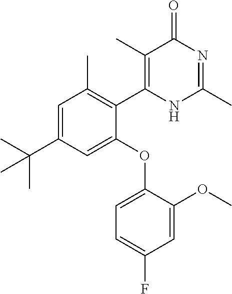 [SMILES: COc1cc(F)ccc1Oc1cc(C(C)(C)C)cc(C)c1-c1[nH]c(C)nc(=O)c1C]

6-[4-tert-butyl-2-(4-fluoro-2-methoxy-phenoxy)-
6-methyl-phenyl]-2,5-dimethyl-1H-pyrimidin-4-
one

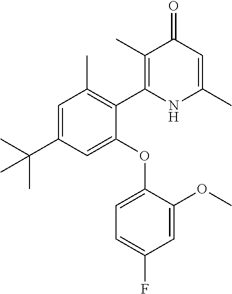 [SMILES: COc1cc(F)ccc1Oc1cc(C(C)(C)C)cc(C)c1-c1[nH]c(C)cc(=O)c1C]

2-[4-tert-butyl-2-(4-fluoro-2-methoxy-phenoxy)-
6-methyl-phenyl]-3,6-dimethyl-1H-pyridin-4-one

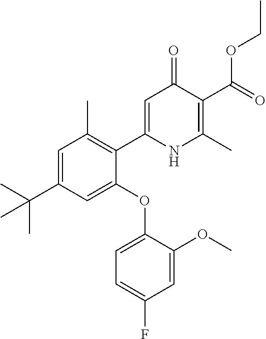 [SMILES: CCOC(=O)c1c(C)[nH]c(-c2c(C)cc(C(C)(C)C)cc2Oc2ccc(F)cc2OC)cc1=O]

ethyl 6-[4-tert-butyl-2-(4-fluoro-2-methoxy-
phenoxy)-6-methyl-phenyl]-2-methyl-4-oxo-1H-
pyridine-3-carboxylate

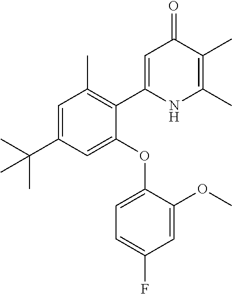 [SMILES: COc1cc(F)ccc1Oc1cc(C(C)(C)C)cc(C)c1-c1cc(=O)c(C)c(C)[nH]1]

6-[4-tert-butyl-2-(4-fluoro-2-methoxy-phenoxy)-
6-methyl-phenyl]-2,3-dimethyl-1H-pyridin-4-one

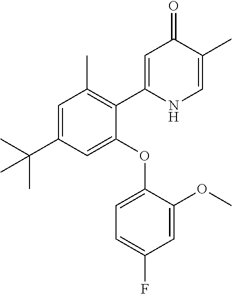 [SMILES: COc1cc(F)ccc1Oc1cc(C(C)(C)C)cc(C)c1-c1cc(=O)c(C)c[nH]1]

2-[4-tert-butyl-2-(4-fluoro-2-methoxy-phenoxy)-
6-methyl-phenyl]-5-methyl-1H-pyridin-4-one

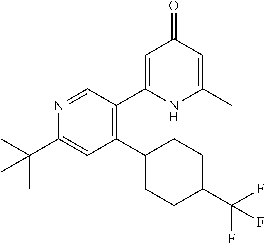 [SMILES: Cc1cc(=O)cc(-c2cnc(C(C)(C)C)cc2C2CCC(C(F)(F)F)CC2)[nH]1]

2-[6-tert-butyl-4-[4-(trifluoromethyl)cyclohexyl]-
3-pyridyl]-6-methyl-1H-pyridin-4-one

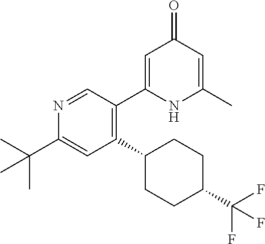 [SMILES: Cc1cc(=O)cc(-c2cnc(C(C)(C)C)cc2[C@H]2CC[C@@H](C(F)(F)F)CC2)[nH]1]

2-[6-tert-butyl-4-((1s,4s)-4-
(trifluoromethyl)cyclohexyl)-3-pyridyl]-6-methyl-
1H-pyridin-4-one

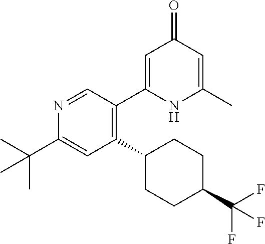 [SMILES: Cc1cc(=O)cc(-c2cnc(C(C)(C)C)cc2[C@H]2CC[C@H](C(F)(F)F)CC2)[nH]1]

2-[6-tert-butyl-4-((1r,4r)-4-
(trifluoromethyl)cyclohexyl]-3-pyridyl]-6-methyl-
1H-pyridin-4-one

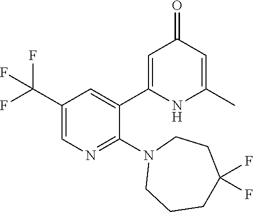 [SMILES: Cc1cc(=O)cc(-c2cc(C(F)(F)F)cnc2N2CCCC(F)(F)CC2)[nH]1]

2-[2-(4,4-difluoroazepan-1-yl)-5-
(trifluoromethyl)-3-pyridyl]-6-methyl-1H-pyridin-
4-one

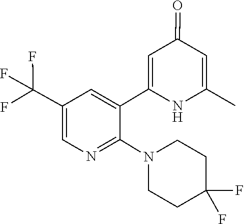 [SMILES: Cc1cc(=O)cc(-c2cc(C(F)(F)F)cnc2N2CCC(F)(F)CC2)[nH]1]

2-[2-(4,4-difluoro-1-piperidyl)-5-
(trifluoromethyl)-3-pyridyl]-6-methyl-1H-pyridin-
4-one

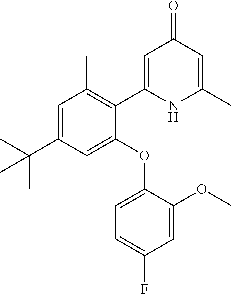 [SMILES: COc1cc(F)ccc1Oc1cc(C(C)(C)C)cc(C)c1-c1cc(=O)cc(C)[nH]1]

2-[4-tert-butyl-2-(4-fluoro-2-methoxy-phenoxy)-
6-methyl-phenyl]-6-methyl-1H-pyridin-4-one

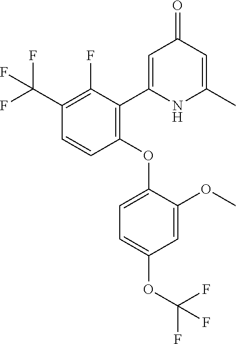 [SMILES: COc1cc(OC(F)(F)F)ccc1Oc1ccc(C(F)(F)F)c(F)c1-c1cc(=O)cc(C)[nH]1]

2-[2-fluoro-6-[2-methoxy-4-
(trifluoromethoxy)phenoxy]-3-
(trifluoromethyl)phenyl]-6-methyl-1H-pyridin-4-
one

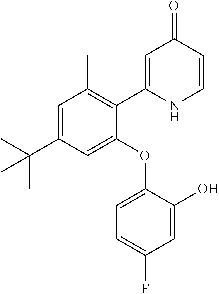 [SMILES: Cc1cc(C(C)(C)C)cc(Oc2ccc(F)cc2O)c1-c1cc(=O)cc[nH]1]

2-[4-tert-butyl-2-(4-fluoro-2-hydroxy-phenoxy)-
6-methyl-phenyl]-1H-pyridin-4-one

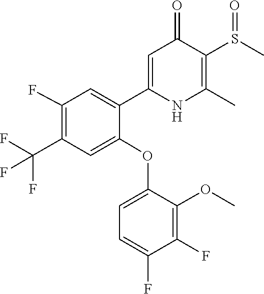 [SMILES: COc1c(Oc2cc(C(F)(F)F)c(F)cc2-c2cc(=O)c(S(C)=O)c(C)[nH]2)ccc(F)c1F]

6-[2-(3,4-difluoro-2-methoxy-phenoxy)-5-fluoro-
4-(trifluromethyl)phenyl]-2-methyl-3-
methylsulfinyl-1H-pyridin-4-one

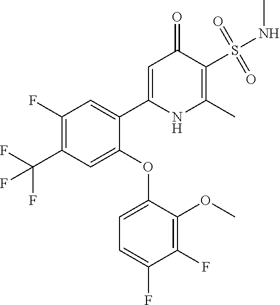 [SMILES: CNS(=O)(=O)c1c(C)[nH]c(-c2cc(F)c(C(F)(F)F)cc2Oc2ccc(F)c(F)c2OC)cc1=O]

6-[2-(3,4-difluoro-2-methoxy-phenoxy)-5-fluoro-
4-(trifluoromethyl)phenyl]-N,2-dimethyl-4-oxo-
1,4-dihydropyridine-3-sulfonamide

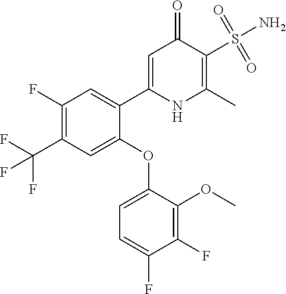 [SMILES: COc1c(Oc2cc(C(F)(F)F)c(F)cc2-c2cc(=O)c(S(N)(=O)=O)c(C)[nH]2)ccc(F)c1F]

6-[2-(3,4-difluoro-2-methoxy-phenoxy)-5-fluoro-
4-(trifluoromethyl)phenyl]-2-methyl-4-oxo-1H-
pyridine-3-sulfonamide

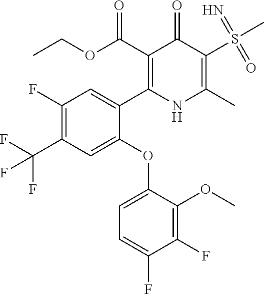 [SMILES: CCOC(=O)c1c(-c2cc(F)c(C(F)(F)F)cc2Oc2ccc(F)c(F)c2OC)[nH]c(C)c(S(C)(=N)=O)c1=O]

ethyl 2-[2-(3,4-difluoro-2-methoxy-phenoxy)-5-
fluoro-4-(trifluoromethyl)phenyl]-6-methyl-5-
(methyslulfonimidoyl)-4-oxo-1H-pyridine-3-
carboxylate

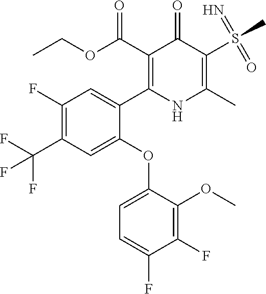 [SMILES: CCOC(=O)c1c(-c2cc(F)c(C(F)(F)F)cc2Oc2ccc(F)c(F)c2OC)[nH]c(C)c([S@](C)(=N)=O)c1=O]

ethyl (R)-2-[2-(3,4-difluoro-2-methoxy-phenoxy)-
5-fluoro-4-(trifluoromethyl)phenyl]-6-methyl-5-
(methylsulfonimidoyl)-4-oxo-1H-pyridine-3-
carboxylate

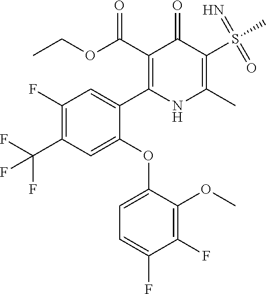 [SMILES: CCOC(=O)c1c(-c2cc(F)c(C(F)(F)F)cc2Oc2ccc(F)c(F)c2OC)[nH]c(C)c([S@@](C)(=N)=O)c1=O]

ethyl (S)-2-[2-(3,4-difluoro-2-methoxy-phenoxy)-
5-fluoro-4-(trifluoromethyl)phenyl]-6-methyl-5-
(methylsulfonimidoyl)-4-oxo-1H-pyridine-3-
carboxylate

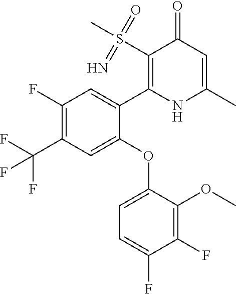 [SMILES: COc1c(Oc2cc(C(F)(F)F)c(F)cc2-c2[nH]c(C)cc(=O)c2S(C)(=N)=O)ccc(F)c1F]

2-[2-(3,4-difluoro-2-methoxy-phenxoy)-5-fluoro-
4-(trifluoromethyl)phenyl]-6-methyl-3-
(methylsulfonimidoyl)-1H-pyridin-4-one

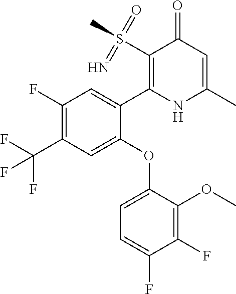 [SMILES: COc1c(Oc2cc(C(F)(F)F)c(F)cc2-c2[nH]c(C)cc(=O)c2[S@](C)(=N)=O)ccc(F)c1F]

(R)-2-[2-(3,4-difluoro-2-methoxy-phenoxy)-5-
fluoro-4-(trifluoromethyl)phenyl]-6-methyl-3-
(methylsulfonimidoyl)-1H-pyridin-4-one

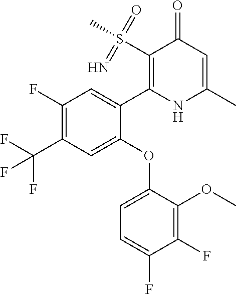 [SMILES: COc1c(Oc2cc(C(F)(F)F)c(F)cc2-c2[nH]c(C)cc(=O)c2[S@@](C)(=N)=O)ccc(F)c1F]

(S)-2-[2-(3,4-difluoro-2-methoxy-phenoxy)-5-
fluoro-4-(trifluoromethyl)phenyl]-6-methyl-3-
(methylsulfonimidoyl)-1H-pyridin-4-one

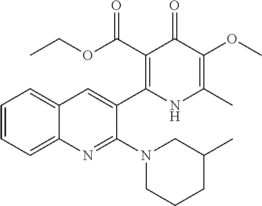 [SMILES: CCOC(=O)c1c(-c2cc3ccccc3nc2N2CCCC(C)C2)[nH]c(C)c(OC)c1=O]

ethyl 5-methoxy-6-methyl-2-[2-[3-methyl-1-
piperidyl]-3-quinolyl]-4-oxo-1H-pyridine-3-
carboxylate

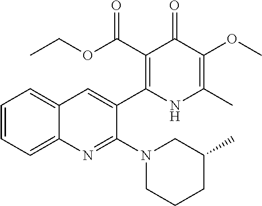 [SMILES: CCOC(=O)c1c(-c2cc3ccccc3nc2N2CCC[C@@H](C)C2)[nH]c(C)c(OC)c1=O]

ethyl 5-methoxy-6-methyl-2-[2-[(3S)-3-methyl-1-
piperidyl]-3-quinolyl]-4-oxo-1H-pyridin-3-
carboxylate

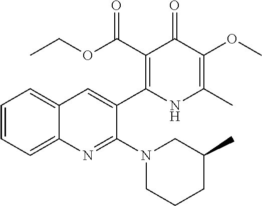 [SMILES: CCOC(=O)c1c(-c2cc3ccccc3nc2N2CCC[C@H](C)C2)[nH]c(C)c(OC)c1=O]

ethyl 5-methoxy-6-methyl-2-[2-[(3R)-3-methyl-1-
piperidyl]-3-quinolyl]-4-oxo-1H-pyridin-3-
carboxylate

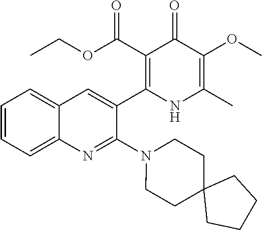 [SMILES: CCOC(=O)c1c(-c2cc3ccccc3nc2N2CCC3(CCCC3)CC2)[nH]c(C)c(OC)c1=O]

ethyl 2-[2-(8-azaspiro[4.5]decan-8-yl)-3-
quinolyl]-5-methoxy-6-methyl-4-oxo-1H-
pyridine-3-carboxylate

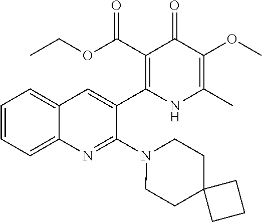 [SMILES: CCOC(=O)c1c(-c2cc3ccccc3nc2N2CCC3(CCC3)CC2)[nH]c(C)c(OC)c1=O]

ethyl 2-[2-(7-azaspiro[3.5]nonan-7-yl)-3-
quinolyl]-5-methoxy-6-methyl-4-oxo-1H-
pyridine-3-carboxylate

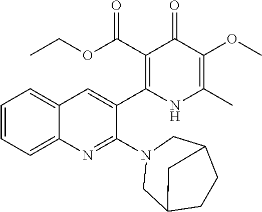 [SMILES: CCOC(=O)c1c(-c2cc3ccccc3nc2N2CC3CCC(C3)C2)[nH]c(C)c(OC)c1=O]

ethyl 2-[2-(3-azabicyclo[3.2.1]octan-3-yl)-3-
quinolyl]-5-methoxy-6-methyl-4-oxo-1H-
pyridine-3-carboxylate

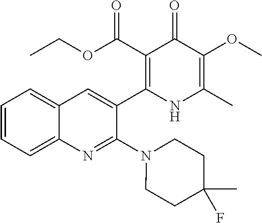 [SMILES: CCOC(=O)c1c(-c2cc3ccccc3nc2N2CCC(C)(F)CC2)[nH]c(C)c(OC)c1=O]

ethyl 2-[2-(4-fluoro-4-methyl-1-piepridyl)-3-
quinolyl]-5-methoxy-6-methyl-4-oxo-1H-
pyridine-3-carboxylate

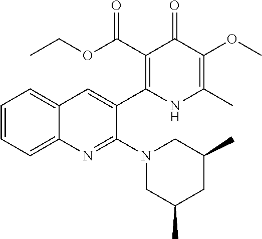 [SMILES: CCOC(=O)c1c(-c2cc3ccccc3nc2N2C[C@H](C)C[C@H](C)C2)[nH]c(C)c(OC)c1=O]

ethyl 2-[2-[(3S,5R)-3,5-dimethyl-1-piperidyl]-3-
quinolyl]-5-methoxy-6-methyl-4-oxo-1H-
pyridine-3-carboxylate

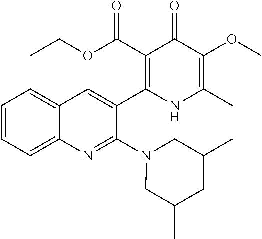 [SMILES: CCOC(=O)c1c(-c2cc3ccccc3nc2N2CC(C)CC(C)C2)[nH]c(C)c(OC)c1=O]

ethyl 2-[2-[3,5-dimethyl-1-piperidyl]-3-quinolyl]-
5-methoxy-6-methyl-4-oxo-1H-pyridine-3-
carboxylate

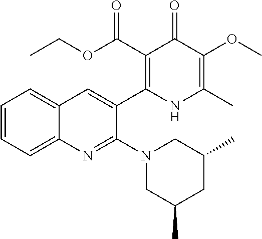 [SMILES: CCOC(=O)c1c(-c2cc3ccccc3nc2N2C[C@H](C)C[C@@H](C)C2)[nH]c(C)c(OC)c1=O]

ethyl 2-[2-[(3R,5R)-3,5-dimethyl-1-piperidyl]-3-
quinolyl]-5-methoxy-6-methyl-4-oxo-1H-
pyridine-3-carboxylate

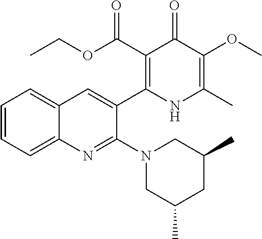 [SMILES: CCOC(=O)c1c(-c2cc3ccccc3nc2N2C[C@@H](C)C[C@H](C)C2)[nH]c(C)c(OC)c1=O]

ethyl 2-[2-[(3S,5S)-3,5-dimethyl-1-piperidyl]-3-
quinolyl]-5-methoxy-6-methyl-4-oxo-1H-
pyridine-3-carboxylate

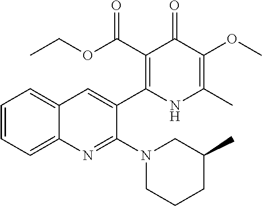 [SMILES: CCOC(=O)c1c(-c2cc3ccccc3nc2N2CCC[C@H](C)C2)[nH]c(C)c(OC)c1=O]

ethyl 5-methoxy-6-methyl-2-[2-[(3R)-3-methyl-1-
piperidyl]-3-quinolyl]-4-oxo-1H-pyridine-3-
carboxylate

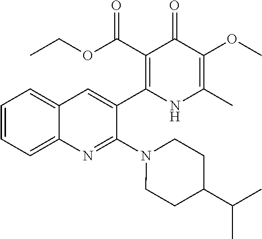 [SMILES: CCOC(=O)c1c(-c2cc3ccccc3nc2N2CCC(C(C)C)CC2)[nH]c(C)c(OC)c1=O]

ethyl 2-[2-(4-isopropyl-1-piperidyl)-3-quinolyl]-
5-methoxy-6-methyl-4-oxo-1H-pyridine-3-
carboxylate

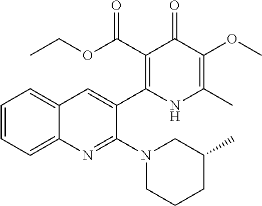 [SMILES: CCOC(=O)c1c(-c2cc3ccccc3nc2N2CCC[C@@H](C)C2)[nH]c(C)c(OC)c1=O]

ethyl 5-methoxy-6-methyl-2-[2-[(3S)-3-methyl-1-
piperidyl]-3-quinolyl]-4-oxo-1H-pyridine-3-
carboxylate

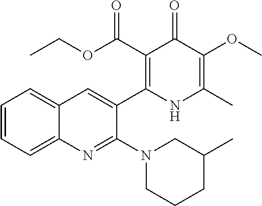 [SMILES: CCOC(=O)c1c(-c2cc3ccccc3nc2N2CCCC(C)C2)[nH]c(C)c(OC)c1=O]

ethyl 5-methoxy-6-methyl-2-[2-[3-methyl-1-
piperidyl]-3-quinolyl]-4-oxo-1H-pyridine-3-
carboxylate

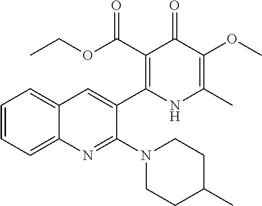 [SMILES: CCOC(=O)c1c(-c2cc3ccccc3nc2N2CCC(C)CC2)[nH]c(C)c(OC)c1=O]

ethyl 5-methoxy-6-methyl-2-[2-(4-methyl-1-
piperidyl)-3-quinolyl]-4-oxo-1H-pyridine-3-
carboxylate

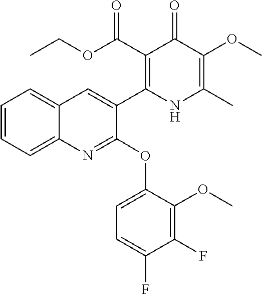 [SMILES: CCOC(=O)c1c(-c2cc3ccccc3nc2Oc2ccc(F)c(F)c2OC)[nH]c(C)c(OC)c1=O]

ethyl 2-[2-(3,4-difluoro-2-methoxy-phenoxy)-3-
quinolyl]-5-methoxy-6-methyl-4-oxo-1H-
pyridine-3-carboxylate

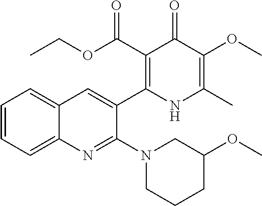 [SMILES: CCOC(=O)c1c(-c2cc3ccccc3nc2N2CCCC(OC)C2)[nH]c(C)c(OC)c1=O]

ethyl 5-methoxy-2-[2-[3-methoxy-1-piperidyl]-3-
quinolyl]-6-methyl-4-oxo-1H-pyridine-3-
carboxylate

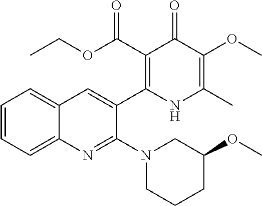 [SMILES: CCOC(=O)c1c(-c2cc3ccccc3nc2N2CCC[C@H](OC)C2)[nH]c(C)c(OC)c1=O]

ethyl 5-methoxy-2-[2-[(3R)-3-methoxy-1-
piperidyl]-3-quinolyl]-6-methyl-4-oxo-1H-
pyridine-3-carboxylate

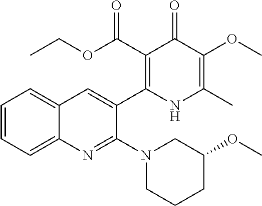 [SMILES: CCOC(=O)c1c(-c2cc3ccccc3nc2N2CCC[C@@H](OC)C2)[nH]c(C)c(OC)c1=O]

ethyl 5-methoxy-2-[2-[(3S)-3-methoxy-1-
piperidyl]-3-quinolyl]-6-methyl-4-oxo-1H-
pyridine-3-carboxylate

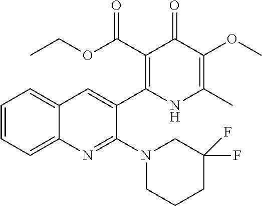 [SMILES: CCOC(=O)c1c(-c2cc3ccccc3nc2N2CCCC(F)(F)C2)[nH]c(C)c(OC)c1=O]

ethyl 2-[2-(3,3-difluoro-1-piperidyl)-3-quinolyl]-
5-methoxy-6-methyl-4-oxo-1H-pyridine-3-
carboxylate

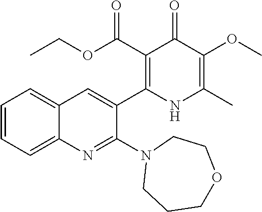 [SMILES: CCOC(=O)c1c(-c2cc3ccccc3nc2N2CCCOCC2)[nH]c(C)c(OC)c1=O]

ethyl 5-methoxy-6-methyl-2-[2-(1,4-oxazepan-4-
yl)-3-quinolyl]-4-oxo-1H-pyridine-3-carboxylate

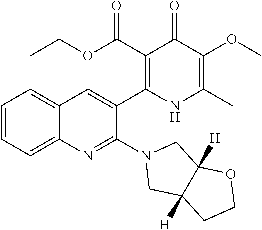 [SMILES: CCOC(=O)c1c(-c2cc3ccccc3nc2N2C[C@H]3CCO[C@H]3C2)[nH]c(C)c(OC)c1=O]

ethyl 2-[2-[(3aS,6aS)-2,3,3a,4,6,6a-
hexahydrofuro[2,3-c]pyrrol-5-yl]-3-quinolyl]-5-
methoxy-6-methyl-4-oxo-1H-pyridine-3-
carboxylate

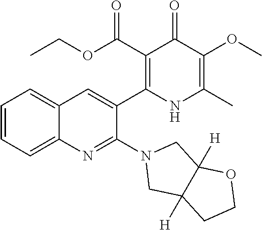 [SMILES: CCOC(=O)c1c(-c2cc3ccccc3nc2N2CC3CCOC3C2)[nH]c(C)c(OC)c1=O]

ethyl 2-[2-[2,3,3a,4,6,6a-hexahydrofuro[2,3-
c]pyrrol-5-yl]-3-quinolyl]-5-methoxy-6-methyl-4-
oxo-1H-pyridine-3-carboxylate

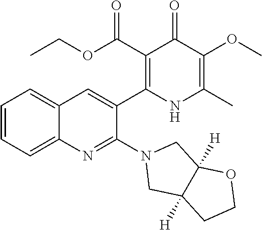 [SMILES: CCOC(=O)c1c(-c2cc3ccccc3nc2N2C[C@@H]3CCO[C@@H]3C2)[nH]c(C)c(OC)c1=O]

ethyl 2-[2-[(3aR,6aR)-3,3,3a,4,6,6a-
hexahydrofuro[2,3-c]pyrrol-5-yl]-3-quinolyl]-5-
methoxy-6-methyl-4-oxo-1H-pyridine-3-
carobxylate

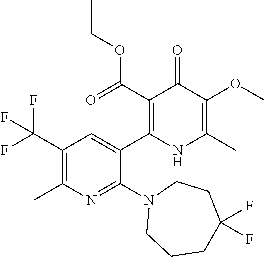 [SMILES: CCOC(=O)c1c(-c2cc(C(F)(F)F)c(C)nc2N2CCCC(F)(F)CC2)[nH]c(C)c(OC)c1=O]

ethyl 2-[2-(4,4-difluoroazepan-1-yl)-6-methyl-5-
(trifluoromethyl)-3-pyridyl]-5-methoxy-6-methyl-
4-oxo-1H-pyridine-3-carobxylate

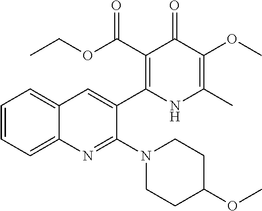 [SMILES: CCOC(=O)c1c(-c2cc3ccccc3nc2N2CCC(OC)CC2)[nH]c(C)c(OC)c1=O]

ethyl 5-methoxy-2-[2-(4-methoxy-1-piperidyl)-3-
quinolyl]-6-methyl-4-oxo-1H-pyridine-3-
carboxylate

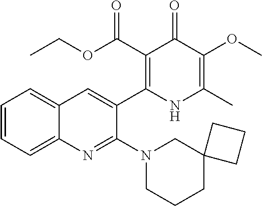 [SMILES: CCOC(=O)c1c(-c2cc3ccccc3nc2N2CCCC3(CCC3)C2)[nH]c(C)c(OC)c1=O]

ethyl 2-[2-(6-azaspiro[3.5]nonan-6-yl)-3-
quinolyl]-5-methoxy-6-methyl-4-oxo-1H-
pyridine-3-carobxylate

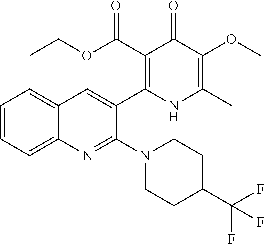 [SMILES: CCOC(=O)c1c(-c2cc3ccccc3nc2N2CCC(C(F)(F)F)CC2)[nH]c(C)c(OC)c1=O]

ethyl 5-methoxy-6-methyl-4-oxo-2-[2-[4-
(trifluoromethyl)-1-piperidyl]-3-quinolyl]-1H-
pyridine-3-carboxylate

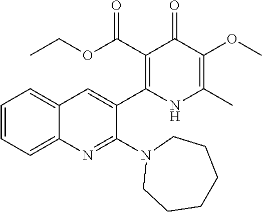 [SMILES: CCOC(=O)c1c(-c2cc3ccccc3nc2N2CCCCCC2)[nH]c(C)c(OC)c1=O]

ethyl 2-[2-(azepan-1-yl)-3-quinolyl]-5-methoxy-
6-methyl-4-oxo-1H-pyridine-3-carboxylate

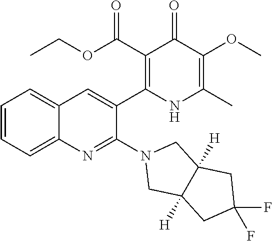 [SMILES: CCOC(=O)c1c(-c2cc3ccccc3nc2N2C[C@@H]3CC(F)(F)C[C@@H]3C2)[nH]c(C)c(OC)c1=O]

ethyl 2-[2-[(3aS,6aR)-5,5-difluoro-1,3,3a,4,6,6a-
hexahydrocyclopenta[c]pyrrol-2-yl]-3-quinolyl]-
5-methoxy-6-methyl-4-oxo-1H-pyridine-3-
carboxylate

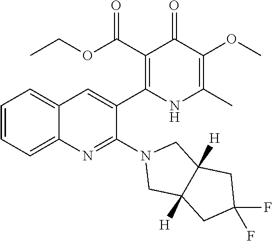 [SMILES: CCOC(=O)c1c(-c2cc3ccccc3nc2N2C[C@@H]3CC(F)(F)C[C@@H]3C2)[nH]c(C)c(OC)c1=O]

ethyl 2-[2-[(3aR,6aS)-,5-difluoro-1,3,3a,4,6,6a-
hexahydrocyclopenta[c]pyrrol-2-yl]-3-quinolyl]-
5-methoxy-6-methyl-4-oxo-1H-pyridine-3-
carboxylate

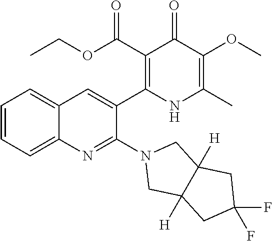 [SMILES: CCOC(=O)c1c(-c2cc3ccccc3nc2N2CC3CC(F)(F)CC3C2)[nH]c(C)c(OC)c1=O]

ethyl 2-[2-[5,5-difluoro-1,3,3a,4,6,6a-
hexahydrocylcopenta[c]pyrrol-2-yl]-3-quinolyl]-
5-methoxy-6-methyl-4-oxo-1H-pyridine-3-
carboxylate

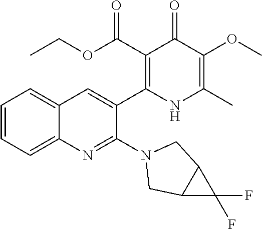 [SMILES: CCOC(=O)c1c(-c2cc3ccccc3nc2N2CC3C(C2)C3(F)F)[nH]c(C)c(OC)c1=O]

ethyl 2-[2-(6,6-difluoro-3-azabicyclo[3.1.0]hexan-
3-yl)-3-quinolyl]-5-methoxy-6-methyl-4-oxo-1H-
pyridine-3-carboxylate

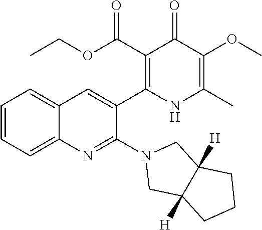 [SMILES: CCOC(=O)c1c(-c2cc3ccccc3nc2N2C[C@H]3CCC[C@H]3C2)[nH]c(C)c(OC)c1=O]

ethyl 2-[2-[(3aR,6aS)-3,3a,4,5,6,6a-hexahydro-
1H-cyclopenta[c]pyrrol-2-yl]-3-quinolyl]-5-
methoxy-6-methyl-4-oxo-1H-pyridine-3-
carboxylate

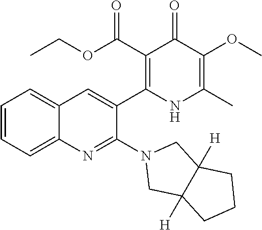 [SMILES: CCOC(=O)c1c(-c2cc3ccccc3nc2N2CC3CCCC3C2)[nH]c(C)c(OC)c1=O]

ethyl 2-[2-[3,3a,4,5,6,6a-hexahydro-1H-
cyclopenta[c]pyrrol-2-yl]-3-quinolyl]-5-methoxy-
6-methyl-4-oxo-1H-pyridine-3-carboxylate

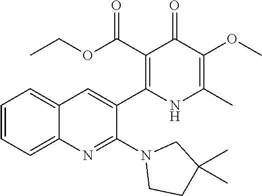 [SMILES: CCOC(=O)c1c(-c2cc3ccccc3nc2N2CCC(C)(C)C2)[nH]c(C)c(OC)c1=O]

ethyl 2-[2-(3,3-dimethylpyrrolidin-1-yl)-3-
quinolyl]-5-methoxy-6-methyl-4-oxo-1H-
pyridine-3-carboxylate

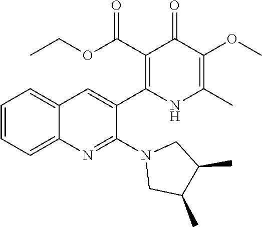 [SMILES: CCOC(=O)c1c(-c2cc3ccccc3nc2N2C[C@@H](C)[C@@H](C)C2)[nH]c(C)c(OC)c1=O]

ethyl 2-[2-[(3S,4R)-3,4-dimethylpyrrolidin-1-yl]-
3-quinolyl]-5-methoxy-6-methyl-4-oxo-1H-
pyridine-3-carboxylate

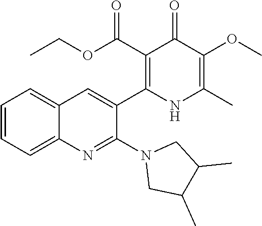 [SMILES: CCOC(=O)c1c(-c2cc3ccccc3nc2N2CC(C)C(C)C2)[nH]c(C)c(OC)c1=O]

ethyl 2-[2-[3,4-dimethylpyrrolidin-1-yl]-3-
quinolyl]-5-methoxy-6-methyl-4-oxo-1H-
pyridine-3-carboxylate

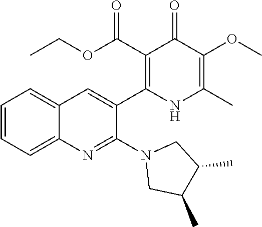 [SMILES: CCOC(=O)c1c(-c2cc3ccccc3nc2N2C[C@H](C)[C@@H](C)C2)[nH]c(C)c(OC)c1=O]

ethyl 2-[2-[(3R,4R)-3,4-dimethylpyrrolidin-1-yl]-
3-quinolyl]-5-methoxy-6-methyl-4-oxo-1H-
pyridine-3-carboxylate

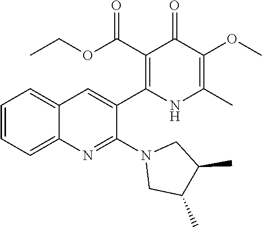 [SMILES: CCOC(=O)c1c(-c2cc3ccccc3nc2N2C[C@@H](C)[C@H](C)C2)[nH]c(C)c(OC)c1=O]

ethyl 2-[2-[(3S,4S)-3,4-dimethylpyrrolidin-1-yl]-
3-quinolyl]-5-methoxy-6-methyl-4-oxo-1H-
pyridine-3-carboxylate

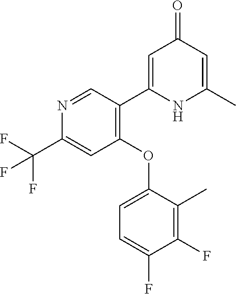 [SMILES: Cc1cc(=O)cc(-c2cnc(C(F)(F)F)cc2Oc2ccc(F)c(F)c2C)[nH]1]

2-[4-(3,4-difluoro-2-methyl-phenoxy)-6-
(trifluoromethyl)-3-pyridyl]-6-methyl-1H-pyridin-
4-one

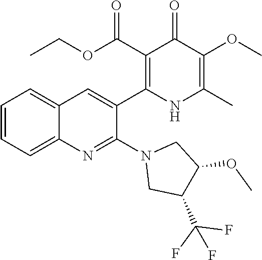 [SMILES: CCOC(=O)c1c(-c2cc3ccccc3nc2N2C[C@H](OC)[C@H](C(F)(F)F)C2)[nH]c(C)c(OC)c1=O]

ethyl 5-methoxy-2-[2-[(3S,4S)-3-methoxy-4-
(trifluoromethyl)pyrrolidin-1-yl]-3-quinolyl]-6-
methyl-4-oxo-1H-pyridine-3-carboxylate

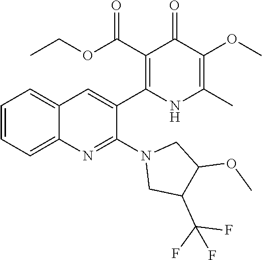 [SMILES: CCOC(=O)c1c(-c2cc3ccccc3nc2N2CC(OC)C(C(F)(F)F)C2)[nH]c(C)c(OC)c1=O]

ethyl 5-methoxy-2-[2-(3-methoxy-4-
(trifluoromethyl)pyrrolidin-1-yl]-3-quinolyl]-6-
methyl-4-oxo-1H-pyridine-3-carboxylate

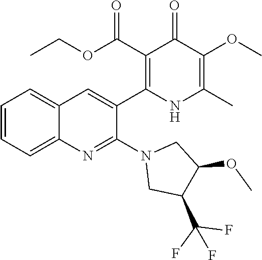 [SMILES: CCOC(=O)c1c(-c2cc3ccccc3nc2N2C[C@@H](OC)[C@@H](C(F)(F)F)C2)[nH]c(C)c(OC)c1=O]

ethyl 5-methoxy-2-[2-[(3R,4R)-3-methoxy-4-
(trifluoromethyl)pyrrolidin-1-yl]-3-quinolyl]-6-
methyl-4-oxo-1H-pyridine-3-carboxylate

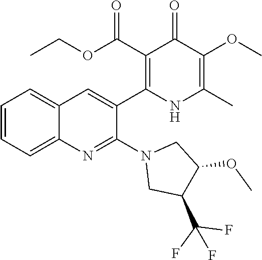 [SMILES: CCOC(=O)c1c(-c2cc3ccccc3nc2N2C[C@H](OC)[C@@H](C(F)(F)F)C2)[nH]c(C)c(OC)c1=O]

ethyl 5-methoxy-2-[2-[(3S,4R)-3-methoxy-4-
(trifluoromethyl)pyrrolidin-1-yl]-3-quinolyl]-6-
methyl-4-oxo-1H-pyridine-3-carboxylate

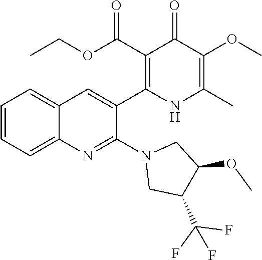 [SMILES: CCOC(=O)c1c(-c2cc3ccccc3nc2N2C[C@@H](C(F)(F)F)[C@H](OC)C2)[nH]c(C)c(OC)c1=O]

ethyl 5-methoxy-2-[2-[(3R,4S)-3-methoxy-4-
(trifluoromethyl)pyrrolidin-1-yl]-3-quinolyl]-6-
methyl-4-oxo-1H-pyridine-3-carboxylate

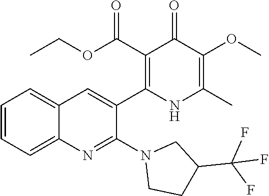 [SMILES: CCOC(=O)c1c(-c2cc3ccccc3nc2N2CCC(C(F)(F)F)C2)[nH]c(C)c(OC)c1=O]

ethyl 5-methoxy-6-methyl-4-oxo-2-[2-[3-
(trifluoromethyl)pyrrolidin-1-yl]-3-quinolyl]-1H-
pyridine-3-carboxylate

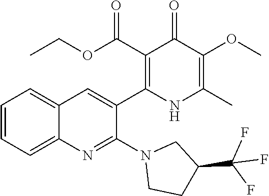 [SMILES: CCOC(=O)c1c(-c2cc3ccccc3nc2N2CC[C@H](C(F)(F)F)C2)[nH]c(C)c(OC)c1=O]

ethyl (S)-5-methoxy-6-methyl-4-oxo-2-[2-[3-
(trifluoromethyl)pyrrolidin-1-yl]-3-quinolyl]-1H-
pyridine-3-carboxylate

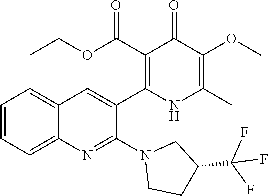 [SMILES: CCOC(=O)c1c(-c2cc3ccccc3nc2N2CC[C@@H](C(F)(F)F)C2)[nH]c(C)c(OC)c1=O]

ethyl (R)-5-methoxy-6-methyl-4-oxo-2-[2-[3-
(trifluoromethyl)pyrrolidin-1-yl]-3-quinolyl]-1H-
pyridine-3-carboxylate

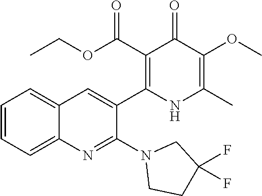 [SMILES: CCOC(=O)c1c(-c2cc3ccccc3nc2N2CCC(F)(F)C2)[nH]c(C)c(OC)c1=O]

ethyl 2-[2-(3,3-difluoropyrrolidin-1-yl)-3-
quinolyl]-5-methoxy-6-methyl-4-oxo-1H-
pyridine-3-carboxylate

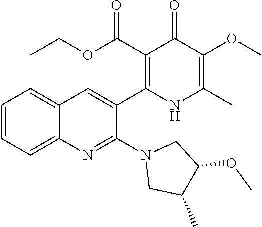 [SMILES: CCOC(=O)c1c(-c2cc3ccccc3nc2N2C[C@@H](C)[C@@H](OC)C2)[nH]c(C)c(OC)c1=O]

ethyl 5-methoxy-2-[2-[(3S,4S)-3-methoxy-4-
methyl-pyrroldiin-1-yl]-3-quinolyl]-6-methyl-4-
oxo-1H-pyridine-3-carboxylate

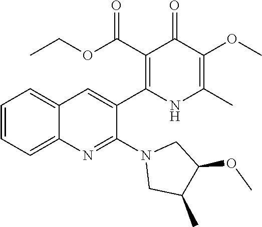 [SMILES: CCOC(=O)c1c(-c2cc3ccccc3nc2N2C[C@H](C)[C@H](OC)C2)[nH]c(C)c(OC)c1=O]

ethyl 5-methoxy-2-[2-[(3R,4R)-3-methoxy-4-
methyl-pyrroldiin-1-yl]-3-quinolyl]-6-methyl-4-
oxo-1H-pyridine-3-carboxylate

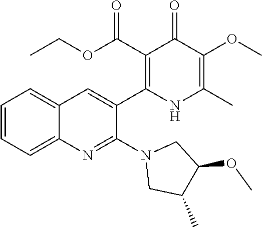 [SMILES: CCOC(=O)c1c(-c2cc3ccccc3nc2N2C[C@@H](C)[C@H](OC)C2)[nH]c(C)c(OC)c1=O]

ethyl 5-methoxy-2-[2-[(3S,4R)-3-methoxy-4-
methyl-pyrrolidin-1-yl]-3-quinolyl]-6-methyl-4-
oxo-1H-pyridine-3-carboxylate

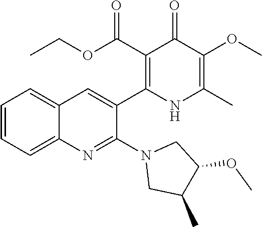 [SMILES: CCOC(=O)c1c(-c2cc3ccccc3nc2N2C[C@H](OC)[C@@H](C)C2)[nH]c(C)c(OC)c1=O]

ethyl 5-methoxy-2-[2-[(3R,4S)-3-methoxy-4-
methyl-pyrrolidin-1-yl]-3-quinolyl]-6-methyl-4-
oxo-1H-pyridine-3-carboxylate

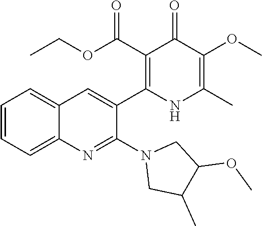 [SMILES: CCOC(=O)c1c(-c2cc3ccccc3nc2N2CC(C)C(OC)C2)[nH]c(C)c(OC)c1=O]

ethyl 5-methoxy-2-[2-[3-methoxy-4-methyl-
pyrrolidin-1-yl]-3-quinolyl]-6-methyl-4-oxo-1H-
pyridine-3-carboxylate

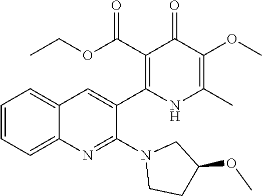 [SMILES: CCOC(=O)c1c(-c2cc3ccccc3nc2N2CC[C@H](OC)C2)[nH]c(C)c(OC)c1=O]

ethyl 5-methoxy-2-[2-[(3R)-3-methoxypyrrolidin-
1-yl]-3-quinolyl]-6-methyl-4-oxo-1H-pyridine-3-
carboxylate

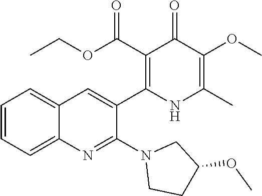 [SMILES: CCOC(=O)c1c(-c2cc3ccccc3nc2N2CC[C@@H](OC)C2)[nH]c(C)c(OC)c1=O]

ethyl 5-methoxy-2-[2-[(3S)-3-methoxypyrrolidin-
1-yl]-3-quinolyl]-6-methyl-4-oxo-1H-pyridine-3-
carboxylate

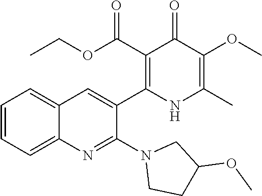 [SMILES: CCOC(=O)c1c(-c2cc3ccccc3nc2N2CCC(OC)C2)[nH]c(C)c(OC)c1=O]

ethyl 5-methoxy-2-[2-[3-methoxypyrrolidin-1-yl]-
3-quinolyl]-6-methyl-4-oxo-1H-pyridine-3-
carboxylate

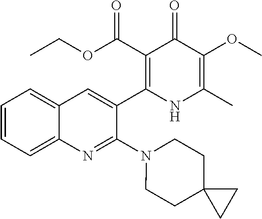 [SMILES: CCOC(=O)c1c(-c2cc3ccccc3nc2N2CCC3(CC2)CC3)[nH]c(C)c(OC)c1=O]

ethyl 2-[2-(6-azaspiro[2.5]octan-6-yl)-3-
quinolyl]-5-methoxy-6-methyl-4-oxo-1H-
pyridine-3-carboxylate

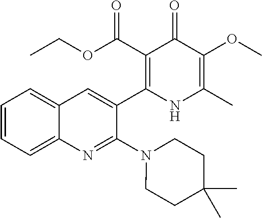 [SMILES: CCOC(=O)c1c(-c2cc3ccccc3nc2N2CCC(C)(C)CC2)[nH]c(C)c(OC)c1=O]

ethyl 2-[2-(4,4-dimethyl-1-piperidyl)-3-quinolyl]-
5-methoxy-6-methyl-4-oxo-1H-pyridine-3-
carboxylate

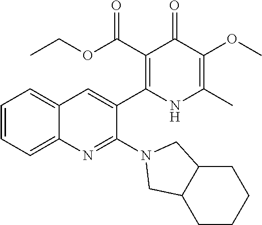 [SMILES: CCOC(=O)c1c(-c2cc3ccccc3nc2N2CC3CCCCC3C2)[nH]c(C)c(OC)c1=O]

ethyl 2-[2-(1,3,3a,4,5,6,7,7a-octahydroisoindol-2-
yl)-3-quinolyl]-5-methoxy-6-methyl-4-oxo-1H-
pyridine-3-carboxylate

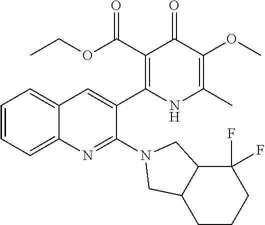 [SMILES: CCOC(=O)c1c(-c2cc3ccccc3nc2N2CC3CCCC(F)(F)C3C2)[nH]c(C)c(OC)c1=O]

ethyl 2-[2-(7,7-difluoro-3,3a,4,5,6,7a-hexahydro-
1H-isoindol-2-yl)-3-quinolyl]-5-methoxy-6-
methyl-4-oxo-1H-pyridine-3-carboxylate

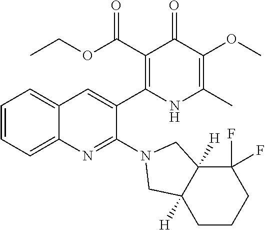 [SMILES: CCOC(=O)c1c(-c2cc3ccccc3nc2N2C[C@@H]3CCCC(F)(F)[C@@H]3C2)[nH]c(C)c(OC)c1=O]

ethyl 2-[2-((3aS,7aR)-7,7-difluroo-3,3a,4,5,6,7a-
hexahydro-1H-isoindol-2-yl)-3-quinolyl]-5-
methoxy-6-methyl-4-oxo-1H-pyridine-3-
carboxylate

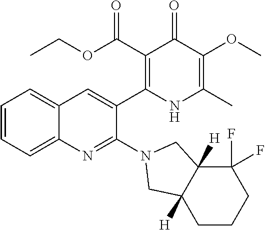 [SMILES: CCOC(=O)c1c(-c2cc3ccccc3nc2N2C[C@H]3CCCC(F)(F)[C@H]3C2)[nH]c(C)c(OC)c1=O]

ethyl 2-[2-((3aR,7aS)-7,7-difluoro-3,3a,4,5,6,7a-
hexahydro-1H-isoindol-2-yl)-3-quinolyl]-5-
methoxy-6-methyl-4-oxo-1H-pyridine-3-
carboxylate

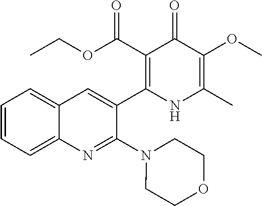 [SMILES: CCOC(=O)c1c(-c2cc3ccccc3nc2N2CCOCC2)[nH]c(C)c(OC)c1=O]

ethyl 5-methoxy-6-methyl-2-(2-morpholino-3-
quinolyl)-4-oxo-1H-pyridine-3-carboxylate

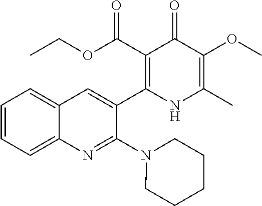 [SMILES: CCOC(=O)c1c(-c2cc3ccccc3nc2N2CCCCC2)[nH]c(C)c(OC)c1=O]

ethyl 5-methoxy-6-methyl-4-oxo-2-[2-(1-
piperidyl)-3-quinolyl]-1H-pyridine-3-carboxylate

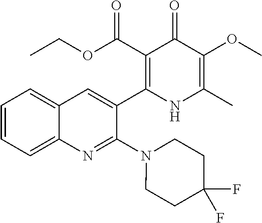 [SMILES: CCOC(=O)c1c(-c2cc3ccccc3nc2N2CCC(F)(F)CC2)[nH]c(C)c(OC)c1=O]

ethyl 5-methoxy-[2-[4,4-difluoro-1-piperidyl]-3-
quinolyl]-6-methyl-4-oxo-1H-pyridine-3-
carboxylate

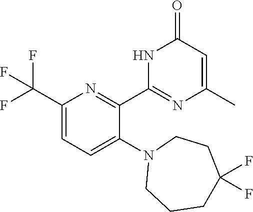 [SMILES: Cc1cc(=O)[nH]c(-c2nc(C(F)(F)F)ccc2N2CCCC(F)(F)CC2)n1]

2-[3-(4,4-difluoroazepan-1-yl)-6-
(trifluoromethyl)-2-pyridyl]-4-methyl-1H-
pyrimidin-6-one

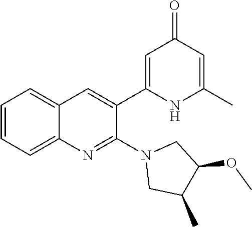 [SMILES: CO[C@@H]1CN(c2nc3ccccc3cc2-c2cc(=O)cc(C)[nH]2)C[C@@H]1C]

2-[2-[(3S,4S)-3-methoxy-4-methyl-pyrrolidin-1-
yl]-3-quinolyl]-6-methyl-1H-pyridin-4-one

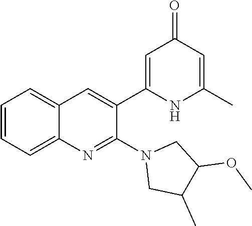 [SMILES: COC1CN(c2nc3ccccc3cc2-c2cc(=O)cc(C)[nH]2)CC1C]

2-[2-[3-methoxy-4-methyl-pyrrolidin-1-yl]-3-
quinolyl]-6-methyl-1H-pyridin-4-one

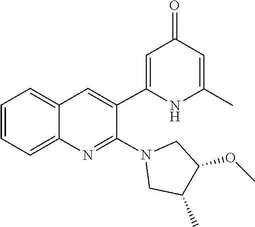 [SMILES: CO[C@H]1CN(c2nc3ccccc3cc2-c2cc(=O)cc(C)[nH]2)C[C@H]1C]

2-[2-[(3R,4R)-3-methoxy-4-methyl-pyrrolidin-1-
yl]-3-quinolyl]-6-methyl-1H-pyridin-4-one

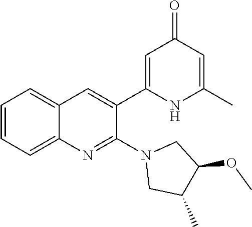 [SMILES: CO[C@@H]1CN(c2nc3ccccc3cc2-c2cc(=O)cc(C)[nH]2)C[C@H]1C]

2-[2-[(3S,4R)-3-methoxy-4-methyl-pyrrolidin-1-
yl]-3-quinolyl]-6-methyl-1H-pyridin-4-one

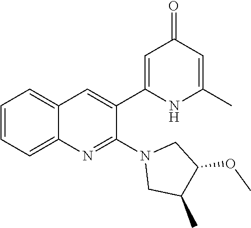 [SMILES: CO[C@H]1CN(c2nc3ccccc3cc2-c2cc(=O)cc(C)[nH]2)C[C@@H]1C]

2-[2-[(3R,4S)-3-methoxy-4-methyl-pyrrolidin-1-
yl]-3-quinolyl]-6-methyl-1H-pyridin-4-one

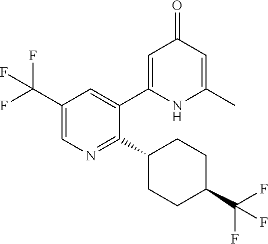 [SMILES: Cc1cc(=O)cc(-c2cc(C(F)(F)F)cnc2[C@H]2CC[C@H](C(F)(F)F)CC2)[nH]1]

6-methyl-5′-(trifluoromethyl)-2′-((1r,4r)-4-
(trifluroomethyl)cyclohexyl)-[2,3′-bipyridin]-
4(1H)-one

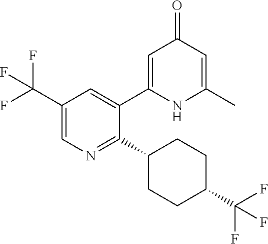 [SMILES: Cc1cc(=O)cc(-c2cc(C(F)(F)F)cnc2[C@H]2CC[C@@H](C(F)(F)F)CC2)[nH]1]

6-methyl-5′-(trifluoromethyl)-2′-((1s,4s)-4-
(trifluoromethyl)cyclohexyl)-[2,3′-bipyridin]-
4(1H)-one

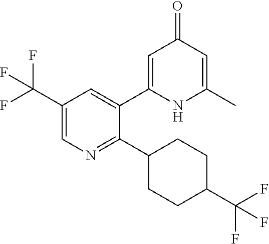 [SMILES: Cc1cc(=O)cc(-c2cc(C(F)(F)F)cnc2C2CCC(C(F)(F)F)CC2)[nH]1]

6-methyl-5′-(trifluoromethyl)-2′-(4-
(trifluoromethyl)cyclohexyl)-[2,3′-bipyridin]-
4(1H)-one

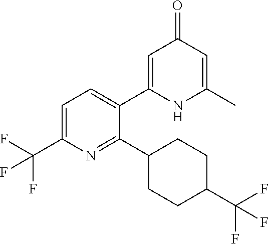 [SMILES: Cc1cc(=O)cc(-c2ccc(C(F)(F)F)nc2C2CCC(C(F)(F)F)CC2)[nH]1]

2-methyl-6-[6-(trifluoromethyl)-2-[4-
(trifluoromethyl)cyclohexyl]-3-pyridyl]-1H-
pyridin-4-one

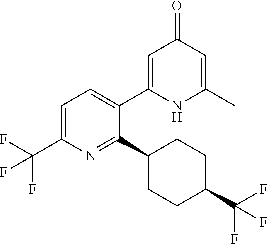 [SMILES: Cc1cc(=O)cc(-c2ccc(C(F)(F)F)nc2[C@H]2CC[C@@H](C(F)(F)F)CC2)[nH]1]

2-methyl-6-[6-(trifluoromethyl)-2-((1s,4s)-4-
(trifluoromethyl)cylcohexyl)-3-pyridyl]-1H-
pyridin-4-one

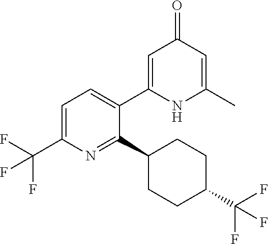 [SMILES: Cc1cc(=O)cc(-c2ccc(C(F)(F)F)nc2[C@H]2CC[C@H](C(F)(F)F)CC2)[nH]1]

2-methyl-6-[6-(trifluoromethyl)-2-((1r,4r)-4-
(trifluoromethyl)cylcohexyl)-3-pyridyl]-1H-
pyridin-4-one

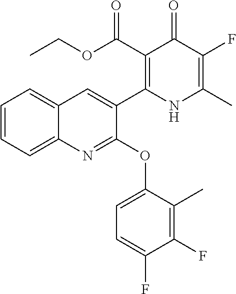 [SMILES: CCOC(=O)c1c(-c2cc3ccccc3nc2Oc2ccc(F)c(F)c2C)[nH]c(C)c(F)c1=O]

ethyl 2-[2-(3,4-difluoro-2-methyl-phenoxy)-3-
quinolyl]-5-fluoro-6-methyl-4-oxo-1H-pyridine-
3-carboxylate

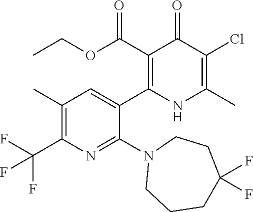 [SMILES: CCOC(=O)c1c(-c2cc(C)c(C(F)(F)F)nc2N2CCCC(F)(F)CC2)[nH]c(C)c(Cl)c1=O]

ethyl 5-chloro-2-[2-(4,4-difluoroazepan-1-yl)-5-
methyl-6-(trifluoromethyl)-3-pyridyl]-6-methyl-4-
oxo-1H-pyridine-3-carboxylate

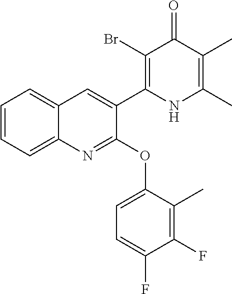 [SMILES: Cc1[nH]c(-c2cc3ccccc3nc2Oc2ccc(F)c(F)c2C)c(Br)c(=O)c1C]

3-bromo-2-[2-(3,4-difluoro-2-methyl-phenoxy)-3-
quinolyl]-5,6-dimethyl-1H-pyridin-4-one

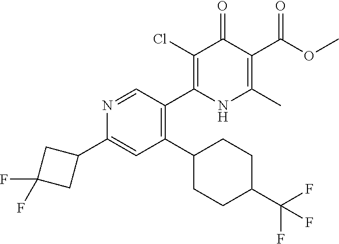 [SMILES: COC(=O)c1c(C)[nH]c(-c2cnc(C3CC(F)(F)C3)cc2C2CCC(C(F)(F)F)CC2)c(Cl)c1=O]

methyl 5-chloro-6-[6-(3,3-difluorocyclobutyl)-4-
[4-(trifluoromethyl)cyclohexyl]-3-pyridyl]-2-
methyl-4-oxo-1H-pyridine-3-carboxylate

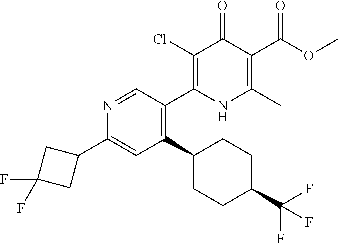 [SMILES: COC(=O)c1c(C)[nH]c(-c2cnc(C3CC(F)(F)C3)cc2[C@H]2CC[C@@H](C(F)(F)F)CC2)c(Cl)c1=O]

methyl 5-chloro-6-[6-(3,3-difluorocyclobutyl)-4-
((1s,4s)-4-(trifluoromethyl)cyclohexyl)-3-
pyridyl]-2-methyl-4-oxo-1H-pyridine-3-
carboxylate

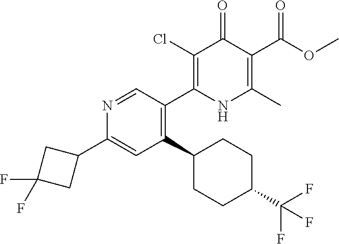 [SMILES: COC(=O)c1c(C)[nH]c(-c2cnc(C3CC(F)(F)C3)cc2[C@H]2CC[C@H](C(F)(F)F)CC2)c(Cl)c1=O]

methyl 5-chloro-6-[6-(3,3-difluorocyclobutyl)-4-
((1r,4r)-4-(trifluromethyl)cyclohexyl)-3-
pyridyl]-2-methyl-4-oxo-1H-pyridine-3-
carboxylate

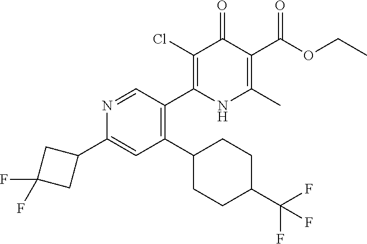 [SMILES: CCOC(=O)c1c(C)[nH]c(-c2cnc(C3CC(F)(F)C3)cc2C2CCC(C(F)(F)F)CC2)c(Cl)c1=O]

ethyl 5-chloro-6-[6-(3,3-difluorocyclobutyl)-4-[4-
(trifluoromethyl)cyclohexyl]-3-pyridyl]-2-methyl-
4-oxo-1H-pyridine-3-carboxylate

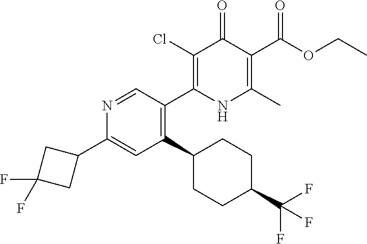 [SMILES: CCOC(=O)c1c(C)[nH]c(-c2cnc(C3CC(F)(F)C3)cc2[C@H]2CC[C@@H](C(F)(F)F)CC2)c(Cl)c1=O]

ethyl 5-chloro-6-[6-(3,3-difluorocyclobutyl)-4-
((1s,4s)-4-(trifluoromethyl)cyclohexyl]-3-
pyridyl]-2-methyl-4-oxo-1H-pyridine-3-
carboxylate

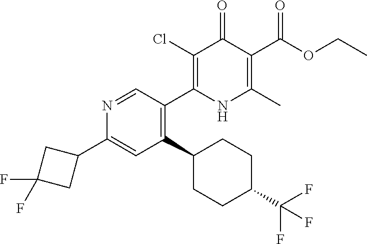 [SMILES: CCOC(=O)c1c(C)[nH]c(-c2cnc(C3CC(F)(F)C3)cc2[C@H]2CC[C@H](C(F)(F)F)CC2)c(Cl)c1=O]

ethyl 5-chloro-6-[6-(3,3-difluorocyclobutyl)-4-
((1r,4r)-4-(trifluoromethyl)cyclohexyl]-3-
pyridyl]-2-methyl-4-oxo-1H-pyridine-3-
carobxylate

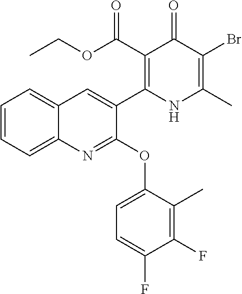 [SMILES: CCOC(=O)c1c(-c2cc3ccccc3nc2Oc2ccc(F)c(F)c2C)[nH]c(C)c(Br)c1=O]

ethyl 5-bromo-2-[2-(3,4-difluoro-2-methyl-
phenoxy)-3-quinolyl]-6-methyl-4-oxo-1H-
pyridine-3-carboxylate

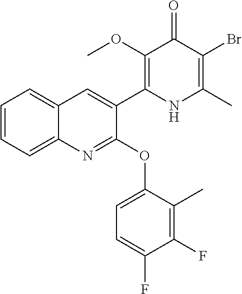 [SMILES: COc1c(-c2cc3ccccc3nc2Oc2ccc(F)c(F)c2C)[nH]c(C)c(Br)c1=O]

5-bromo-2-[2-(3,4-difluoro-2-methyl-phenoxy)-3-
quinolyl]-3-methoxy-6-methyl-1H-pyridin-4-one

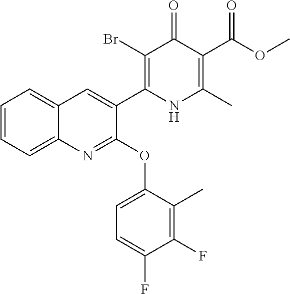 [SMILES: COC(=O)c1c(C)[nH]c(-c2cc3ccccc3nc2Oc2ccc(F)c(F)c2C)c(Br)c1=O]

methyl 5-bromo-6-[2-(3,4-difluoro-2-methyl-
phenoxy)-3-quinolyl]-2-methyl-4-oxo-1H-
pyridine-3-carboxylate

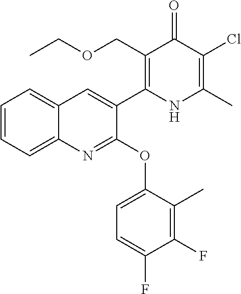 [SMILES: CCOCc1c(-c2cc3ccccc3nc2Oc2ccc(F)c(F)c2C)[nH]c(C)c(Cl)c1=O]

5-chloro-2-[2-(3,4-difluoro-2-methyl-phenoxy)-3-
quinolyl]-3-(ethoxymethyl)-6-methyl-1H-pyridin-
4-one

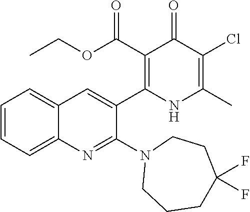 [SMILES: CCOC(=O)c1c(-c2cc3ccccc3nc2N2CCCC(F)(F)CC2)[nH]c(C)c(Cl)c1=O]

ethyl 5-chloro-2-[2-(4,4-difluoroazepan-1-yl)-3-
quinolyl]-6-methyl-4-oxo-1H-pyridine-3-
carboxylate

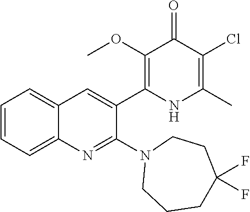 [SMILES: COc1c(-c2cc3ccccc3nc2N2CCCC(F)(F)CC2)[nH]c(C)c(Cl)c1=O]

5-chloro-2-[2-(4,4-difluoroazepan-1-yl)-3-
quinolyl]-3-methoxy-6-methyl-1H-pyridin-4-one

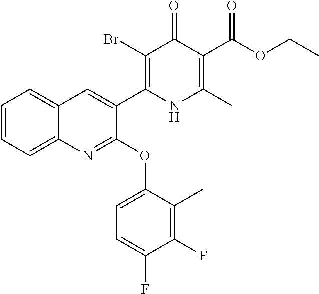 [SMILES: CCOC(=O)c1c(C)[nH]c(-c2cc3ccccc3nc2Oc2ccc(F)c(F)c2C)c(Br)c1=O]

ethyl 5-bromo-6-[2-(3,4-difluoro-2-methyl-
phenoxy)-3-quinolyl]-2-methyl-4-oxo-1H-
pyridine-3-carboxylate

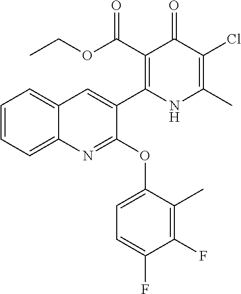 [SMILES: CCOC(=O)c1c(-c2cc3ccccc3nc2Oc2ccc(F)c(F)c2C)[nH]c(C)c(Cl)c1=O]

ethyl 5-chloro-2-[2-(3,4-difluoro-2-methyl-
phenoxy)-3-quinolyl]-6-methyl-4-oxo-1H-
pyridine-3-carboxylate

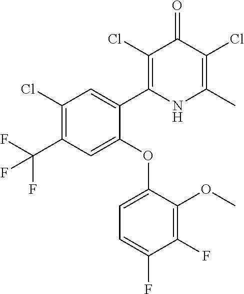 [SMILES: COc1c(Oc2cc(C(F)(F)F)c(Cl)cc2-c2[nH]c(C)c(Cl)c(=O)c2Cl)ccc(F)c1F]

3,5-dichloro-2-[5-chloro-2-(3,4-difluoro-2-
methoxy-phenoxy)-4-(trifluoromethyl)phenyl]-6-
methyl-1H-pyridin-4-one

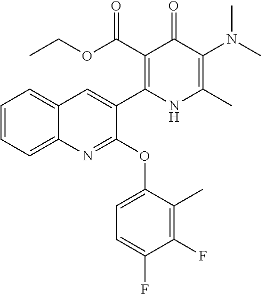 [SMILES: CCOC(=O)c1c(-c2cc3ccccc3nc2Oc2ccc(F)c(F)c2C)[nH]c(C)c(N(C)C)c1=O]

ethyl 2-[2-(3,4-difluoro-2-methyl-phenoxy)-3-
quinolyl]-5-(dimethylamino)-6-methyl-4-oxo-1H-
pyridine-3-carboxylate

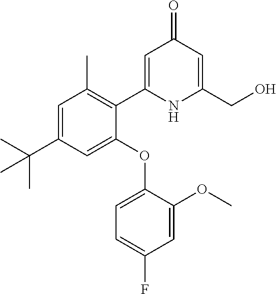 [SMILES: COc1cc(F)ccc1Oc1cc(C(C)(C)C)cc(C)c1-c1cc(=O)cc(CO)[nH]1]

2-[4-tert-butyl-2-(4-fluoro-2-methoxy-phenoxy)-
6-methyl-phenyl]-6-(hydroxymethyl)-1H-pyridin-
4-one

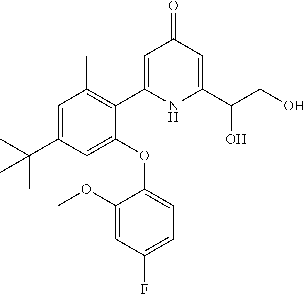 [SMILES: COc1cc(F)ccc1Oc1cc(C(C)(C)C)cc(C)c1-c1cc(=O)cc(C(O)CO)[nH]1]

2-[4-tert-butyl-2-(4-fluoro-2-methoxy-phenoxy)-
6-methyl-phenyl]-6-(1,2-dihydroxyethyl)-1H-
pyridin-4-one

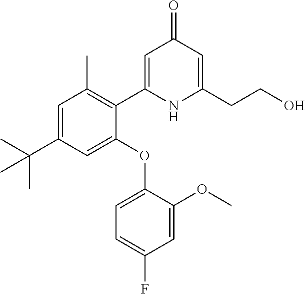 [SMILES: COc1cc(F)ccc1Oc1cc(C(C)(C)C)cc(C)c1-c1cc(=O)cc(CCO)[nH]1]

2-[4-tert-butyl-2-(4-fluoro-2-methoxy-phenoxy)-
6-methyl-phenyl]-6-(2-hydroxyethyl)-1H-pyridin-
4-one

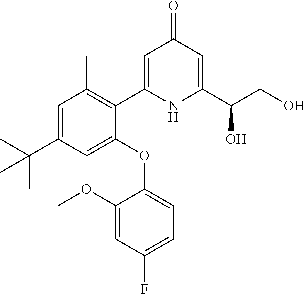 [SMILES: COc1cc(F)ccc1Oc1cc(C(C)(C)C)cc(C)c1-c1cc(=O)cc([C@H](O)CO)[nH]1]

(S)-2-[4-tert-butyl-2-(4-fluoro-2-methoxy-
phenoxy)-6-methyl-phenyl]-6-(1,2-
dihydroxyethyl)-1H-pyridin-4-one

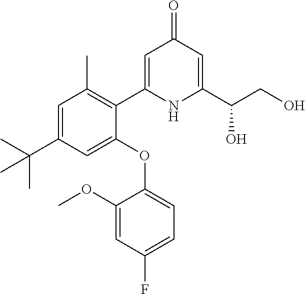 [SMILES: COc1cc(F)ccc1Oc1cc(C(C)(C)C)cc(C)c1-c1cc(=O)cc([C@@H](O)CO)[nH]1]

(R)-2-[4-tert-butyl-2-(4-fluoro-2-methoxy-
phenoxy)-6-methyl-phenyl]-6-(1,2-
dihydroxyethyl)-1H-pyridin-4-one

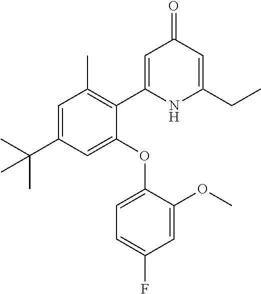 [SMILES: CCc1cc(=O)cc(-c2c(C)cc(C(C)(C)C)cc2Oc2ccc(F)cc2OC)[nH]1]

2-[4-tert-butyl-2-(4-fluoro-2-methoxy-phenoxy)-
6-methyl-phenyl]-6-ethyl-1H-pyridin-4-one

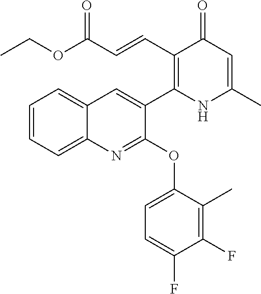 [SMILES: CCOC(=O)/C=C/c1c(-c2cc3ccccc3nc2Oc2ccc(F)c(F)c2C)[nH]c(C)cc1=O]

ethyl (E)-3-[2-[2-(3,4-difluroo-2-methyl-
phenoxy)-3-quinolyl]-6-methyl-4-oxo-1H-
pyridin-3-yl]prop-2-enoate

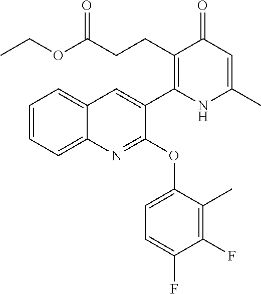 [SMILES: CCOC(=O)CCc1c(-c2cc3ccccc3nc2Oc2ccc(F)c(F)c2C)[nH]c(C)cc1=O]

ethyl 3-[2-[2-(3,4-difluoro-2-methyl-phenoxy)-3-
quinolyl]-6-methyl-4-oxo-1H-pyridin-3-
yl]propanoate

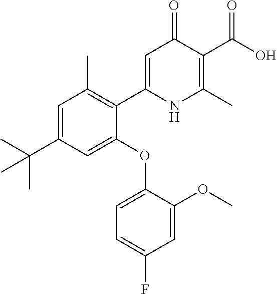 [SMILES: COc1cc(F)ccc1Oc1cc(C(C)(C)C)cc(C)c1-c1cc(=O)c(C(=O)O)c(C)[nH]1]

6-[4-tert-butyl-2-(4-fluoro-2-methoxy-phenoxy)-
6-methyl-phenyl]-2-methyl-4-oxo-1H-pyridine-3-
carboxylic acid

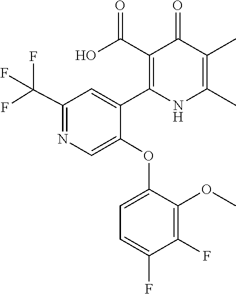 [SMILES: COc1c(Oc2cnc(C(F)(F)F)cc2-c2[nH]c(C)c(C)c(=O)c2C(=O)O)ccc(F)c1F]

2-[5-(3,4-difluoro-2-methoxy-phenoxy)-2-
(trifluoromethyl)-4-pyridyl]-5,6-dimethyl-4-oxo-
1H-pyridine-3-carboxylic acid

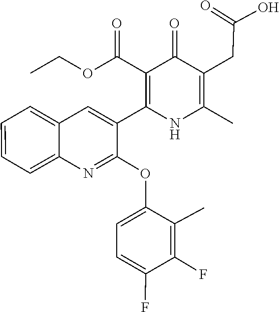 [SMILES: CCOC(=O)c1c(-c2cc3ccccc3nc2Oc2ccc(F)c(F)c2C)[nH]c(C)c(CC(=O)O)c1=O]

2-[6-[2-(3,4-difluoro-2-methyl-phenoxy)-3-
quinolyl]-5-ethoxycarbonyl]-2-methyl-4-oxo-1H-
pyridin-3-yl]acetic acid

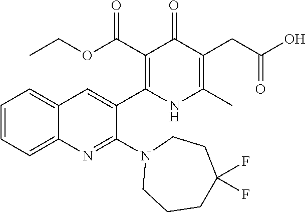 [SMILES: CCOC(=O)c1c(-c2cc3ccccc3nc2N2CCCC(F)(F)CC2)[nH]c(C)c(CC(=O)O)c1=O]

2-[6-[2-(4,4-difluoroazepan-1-yl)-3-quinolyl]-5-
ethoxycarbonyl-2-methyl-4-oxo-1H-pyridin-3-
yl]acetic acid

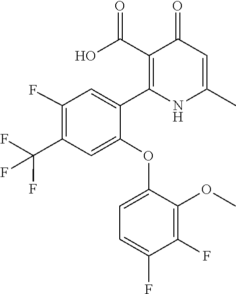 [SMILES: COc1c(Oc2cc(C(F)(F)F)c(F)cc2-c2[nH]c(C)cc(=O)c2C(=O)O)ccc(F)c1F]

2-[2-(3,4-difluoro-2-methoxy-phenoxy)-5-fluoro-
4-(trifluoromethyl)phenyl]-6-methyl-4-oxo-1H-
pyridine-3-carboxylic acid

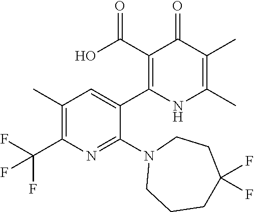 [SMILES: Cc1cc(-c2[nH]c(C)c(C)c(=O)c2C(=O)O)c(N2CCCC(F)(F)CC2)nc1C(F)(F)F]

2-[2-(4,4-difluoroazepan-1-yl)-5-methyl-6-
(trifluoromethyl)-3-pyridyl]-5,6-dimethyl-4-oxo-
1H-pyridine-3-carboxylic acid

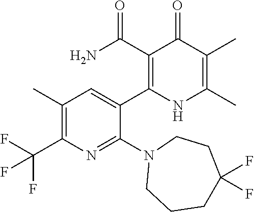 [SMILES: Cc1cc(-c2[nH]c(C)c(C)c(=O)c2C(N)=O)c(N2CCCC(F)(F)CC2)nc1C(F)(F)F]

2-[2-(4,4-difluoroazepan-1-yl)-5-methyl-6-
(trifluoromethyl)-3-pyridyl]-5,6-dimethyl-4-oxo-
1H-pyridine-3-carboxamide

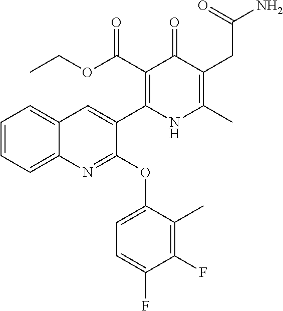 [SMILES: CCOC(=O)c1c(-c2cc3ccccc3nc2Oc2ccc(F)c(F)c2C)[nH]c(C)c(CC(N)=O)c1=O]

ethyl 5-(2-amino-2-oxo-ethyl)-2-[2-(3,4-difluoro-
2-methyl-phenoxy)-3-quinolyl]-6-methyl-4-oxo-
1H-pyridine-3-carboxylate

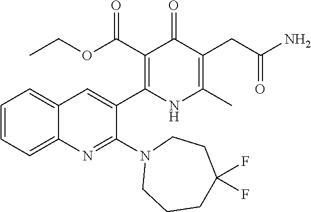 [SMILES: CCOC(=O)c1c(-c2cc3ccccc3nc2N2CCCC(F)(F)CC2)[nH]c(C)c(CC(N)=O)c1=O]

ethyl 5-(2-amino-2-oxo-ethyl)-2-[2-(4,4-
difluoroazepan-1-yl)-3-quinolyl]-6-methyl-4-oxo-
1H-pyridine-3-carboxylate

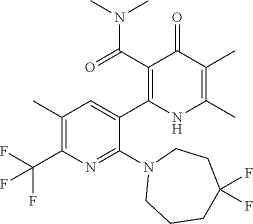 [SMILES: Cc1cc(-c2[nH]c(C)c(C)c(=O)c2C(=O)N(C)C)c(N2CCCC(F)(F)CC2)nc1C(F)(F)F]

2-[2-(4,4-difluoroazepan-1-yl)-5-methyl-6-
(trifluoromethyl)-3-pyridyl]-N,N,5,6-tetramethyl-
4-oxo-1H-pyridine-3-carboxamide

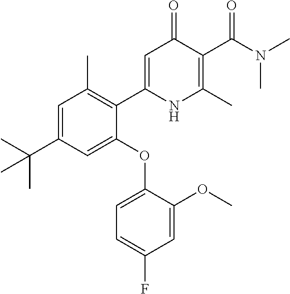 [SMILES: COc1cc(F)ccc1Oc1cc(C(C)(C)C)cc(C)c1-c1cc(=O)c(C(=O)N(C)C)c(C)[nH]1]

6-[4-tert-butyl-2-(4-fluoro-2-methoxy-phenoxy)-
6-methyl-phenyl]-N,N,2-trimethyl-4-oxo-1H-
pyridine-3-carboxamide

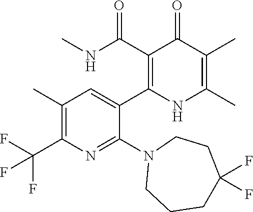 [SMILES: CNC(=O)c1c(-c2cc(C)c(C(F)(F)F)nc2N2CCCC(F)(F)CC2)[nH]c(C)c(C)c1=O]

2-[2-(4,4-difluoroazepan-1-yl)-5-methyl-6-
(trifluoromethyl)-3-pyridyl]-N,5,6-trimethyl-4-
oxo-1H-pyridine-3-carboxamide

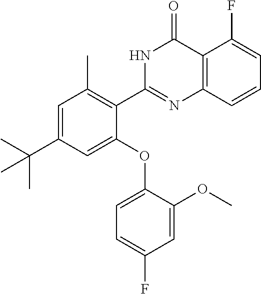 [SMILES: COc1cc(F)ccc1Oc1cc(C(C)(C)C)cc(C)c1-c1nc2cccc(F)c2c(=O)[nH]1]

6-[4-tert-butyl-2-(4-fluoro-2-methoxy-phenoxy)-
6-methyl-phenyl]-2-methyl-4-oxo-1H-pyridine-3-
carboxamide

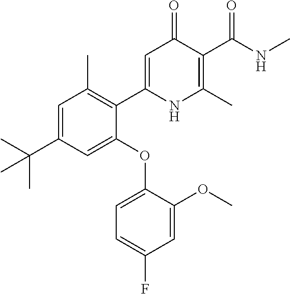 [SMILES: CNC(=O)c1c(C)[nH]c(-c2c(C)cc(C(C)(C)C)cc2Oc2ccc(F)cc2OC)cc1=O]

6-[4-tert-butyl-2-(4-fluoro-2-methoxy-phenoxy)-
6-methyl-phenyl]-N,2-dimethyl-4-oxo-1H-
pyridine-3-carboxamide

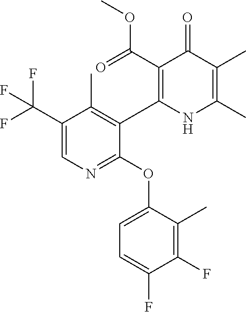 [SMILES: COC(=O)c1c(-c2c(Oc3ccc(F)c(F)c3C)ncc(C(F)(F)F)c2C)[nH]c(C)c(C)c1=O]

methyl 2-[2-(3,4-difluoro-2-methyl-phenoxy)-4-
methyl-5-(trifluoromethyl)-3-pyridyl]-5,6-
dimethyl-4-oxo-1H-pyridine-3-carboxylate

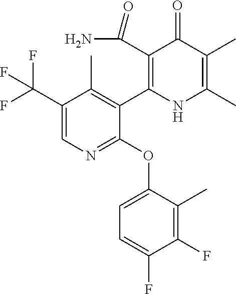 [SMILES: Cc1[nH]c(-c2c(Oc3ccc(F)c(F)c3C)ncc(C(F)(F)F)c2C)c(C(N)=O)c(=O)c1C]

2-[2-(3,4-difluoro-2-methyl-phenoxy)-4-methyl-
5-(trifluoromethyl)-3-pyridyl]-5,6-dimethyl-4-
oxo-1H-pyridine-3-carboxamide

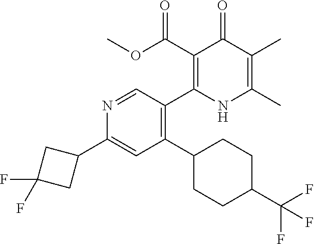 [SMILES: COC(=O)c1c(-c2cnc(C3CC(F)(F)C3)cc2C2CCC(C(F)(F)F)CC2)[nH]c(C)c(C)c1=O]

methyl 2-[6-(3,3-difluorocyclobutyl)-4-[4-
(trifluoromethyl)cyclohexyl]-3-pyridyl]-5,6-
dimethyl-4-oxo-1H-pyridine-3-carboxylate

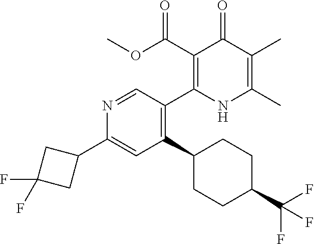 [SMILES: COC(=O)c1c(-c2cnc(C3CC(F)(F)C3)cc2[C@H]2CC[C@@H](C(F)(F)F)CC2)[nH]c(C)c(C)c1=O]

methyl 2-[6-(3,3-difluorocyclobutyl)-4-((1s,4s)-4-
(trifluoromethyl)cyclohexyl)-3-pyridyl]-5,6-
dimethyl-4-oxo-1H-pyridine-3-carboxylate

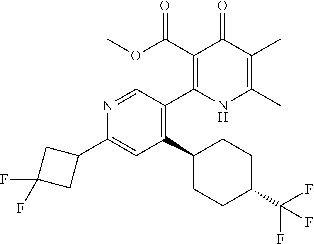 [SMILES: COC(=O)c1c(-c2cnc(C3CC(F)(F)C3)cc2[C@H]2CC[C@H](C(F)(F)F)CC2)[nH]c(C)c(C)c1=O]

methyl 2-[6-(3,3-difluorocyclobutyl)-4-((1r,4r)-4-
(trifluoromethyl)cyclohexyl)-3-pyridyl]-5,6-
dimethyl-4-oxo-1H-pyridine-3-carboxylate

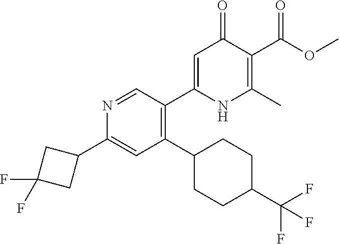 [SMILES: COC(=O)c1c(C)[nH]c(-c2cnc(C3CC(F)(F)C3)cc2C2CCC(C(F)(F)F)CC2)cc1=O]

methyl 6-[6-(3,3-difluorocyclobutyl)-4-[4-
(trifluoromethyl)cyclohexyl]-3-pyridyl]-2-methyl-
4-oxo-1H-pyridine-3-carboxylate

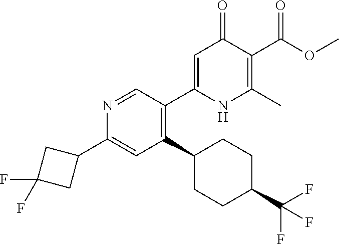 [SMILES: COC(=O)c1c(C)[nH]c(-c2cnc(C3CC(F)(F)C3)cc2[C@H]2CC[C@@H](C(F)(F)F)CC2)cc1=O]

methyl 6-[6-(3,3-difluorocyclobutyl)-4-((1s,4s)-4-
(trifluoromethyl)cyclohexyl)-3-pyridyl]-2-methyl-
4-oxo-1H-pyridine-3-carboxylate

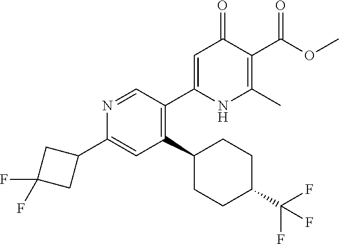 [SMILES: COC(=O)c1c(C)[nH]c(-c2cnc(C3CC(F)(F)C3)cc2[C@H]2CC[C@H](C(F)(F)F)CC2)cc1=O]

methyl 6-[6-(3,3-difluorocyclobutyl)-4-((1r,4r)-4-
(trifluoromethyl)cyclohexyl)-3-pyridyl]-2-methyl-
4-oxo-1H-pyridine-3-carboxylate

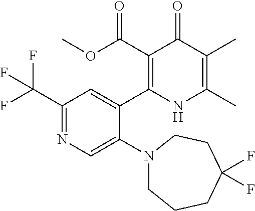 [SMILES: COC(=O)c1c(-c2cc(C(F)(F)F)ncc2N2CCCC(F)(F)CC2)[nH]c(C)c(C)c1=O]

methyl 2-[5-(4,4-difluoroazepan-1-yl)-2-
(trifluoromethyl)-4-pyridyl]-5,6-dimethyl-4-oxo-
1H-pyridine-3-carboxylate

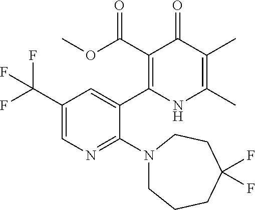 [SMILES: COC(=O)c1c(-c2cc(C(F)(F)F)cnc2N2CCCC(F)(F)CC2)[nH]c(C)c(C)c1=O]

methyl 2-[2-(4,4-difluoroazepan-1-yl)-5-
(trifluoromethyl)-3-pyridyl]-5,6-dimethyl-4-oxo-
1H-pyridine-3-carboxylate

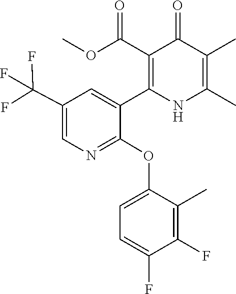 [SMILES: COC(=O)c1c(-c2cc(C(F)(F)F)cnc2Oc2ccc(F)c(F)c2C)[nH]c(C)c(C)c1=O]

methyl 2-[2-(3,4-difluoro-2-methyl-phenoxy)-5-
(trifluoromethyl)-3-pyridyl]-5,6-dimethyl-4-oxo-
1H-pyridine-3-carboxylate

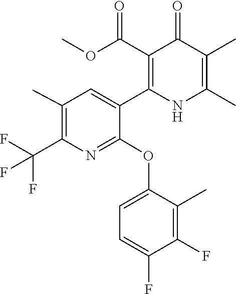 [SMILES: COC(=O)c1c(-c2cc(C)c(C(F)(F)F)nc2Oc2ccc(F)c(F)c2C)[nH]c(C)c(C)c1=O]

methyl 2-[2-(3,4-difluoro-2-methyl-phenoxy)-5-
methyl-6-(trifluoromethyl)-3-pyridyl]-5,6-
dimethyl-4-oxo-1H-pyridine-3-carboxylate

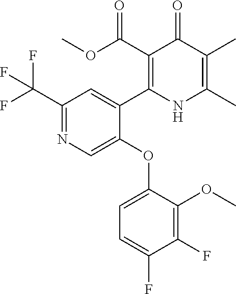 [SMILES: COC(=O)c1c(-c2cc(C(F)(F)F)ncc2Oc2ccc(F)c(F)c2OC)[nH]c(C)c(C)c1=O]

methyl 2-[5-(3,4-difluoro-2-methoxy-phenoxy)-2-
(trifluoromethyl)-4-pyridyl]-5,6-dimethyl-4-oxo-
1H-pyridine-3-carboxylate

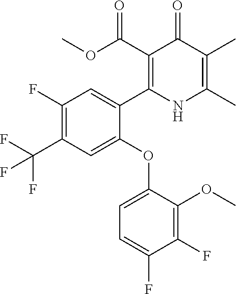 [SMILES: COC(=O)c1c(-c2cc(F)c(C(F)(F)F)cc2Oc2ccc(F)c(F)c2OC)[nH]c(C)c(C)c1=O]

methyl 2-[2-(3,4-difluoro-2-methoxy-phenoxy)-5-
fluoro-4-(trifluoromethyl)phenyl]-5,6-dimethyl-4-
oxo-1H-pyridine-3-carboxylate

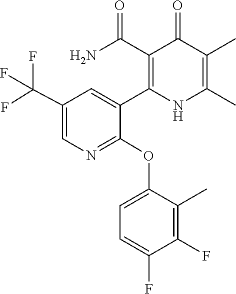 [SMILES: Cc1[nH]c(-c2cc(C(F)(F)F)cnc2Oc2ccc(F)c(F)c2C)c(C(N)=O)c(=O)c1C]

2-[2-(3,4-difluoro-2-methyl-phenoxy)-5-
(trifluoromethyl)-3-pyridyl]-5,6-dimethyl-4-oxo-
1H-pyridine-3-carboxamide

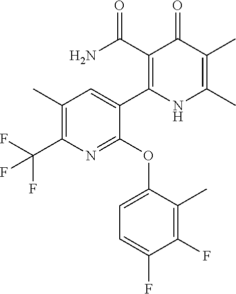 [SMILES: Cc1cc(-c2[nH]c(C)c(C)c(=O)c2C(N)=O)c(Oc2ccc(F)c(F)c2C)nc1C(F)(F)F]

2-[2-(3,4-difluoro-2-methyl-phenoxy)-5-methyl-
6-(trifluoromethyl)-3-pyridyl]-5,6-dimethyl-4-
oxo-1H-pyridine-3-carboxamide

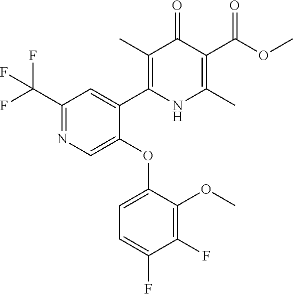 [SMILES: COC(=O)c1c(C)[nH]c(-c2cc(C(F)(F)F)ncc2Oc2ccc(F)c(F)c2OC)c(C)c1=O]

methyl 6-[5-(3,4-difluoro-2-methoxy-phenoxy)-2-
(trifluoromethyl)-4-pyridyl]-2,5-dimethyl-4-oxo-
1H-pyridine-3-carboxylate

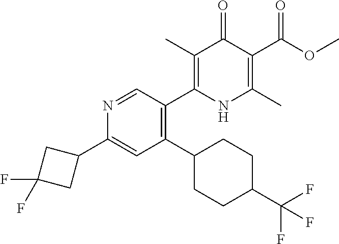 [SMILES: COC(=O)c1c(C)[nH]c(-c2cnc(C3CC(F)(F)C3)cc2C2CCC(C(F)(F)F)CC2)c(C)c1=O]

methyl 6-[6-(3,3-difluorocyclobutyl)-4-[4-
(trifluoromethyl)cyclohexyl]-3-pyridyl]-2,5-
dimethyl-4-oxo-1H-pyridine-3-carboxylate

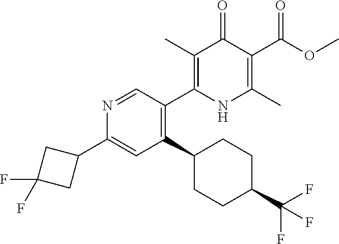 [SMILES: COC(=O)c1c(C)[nH]c(-c2cnc(C3CC(F)(F)C3)cc2[C@H]2CC[C@@H](C(F)(F)F)CC2)c(C)c1=O]

methyl 6-[6-(3,3-difluorocyclobutyl)-4-((1s,4s)-4-
(trifluoromethyl)cyclohexyl)-3-pyridyl]-2,5-
dimethyl-4-oxo-1H-pyridine-3-carboxylate

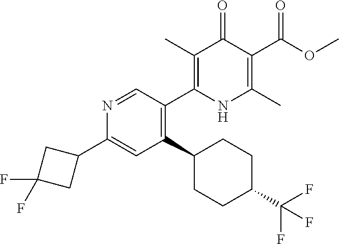 [SMILES: COC(=O)c1c(C)[nH]c(-c2cnc(C3CC(F)(F)C3)cc2[C@H]2CC[C@H](C(F)(F)F)CC2)c(C)c1=O]

methyl 6-[6-(3,3-difluorocyclobutyl)-4-((1r,4r)-4-
(trifluoromethyl)cyclohexyl)-3-pyridyl]-2,5-
dimethyl-4-oxo-1H-pyridine-3-carboxylate

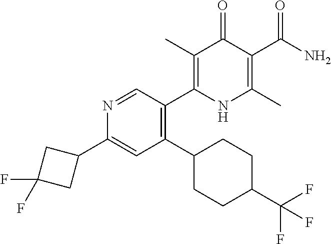 [SMILES: Cc1[nH]c(-c2cnc(C3CC(F)(F)C3)cc2C2CCC(C(F)(F)F)CC2)c(C)c(=O)c1C(N)=O]

6-[6-(3,3-difluorocyclobutyl)-4-[4-
(trifluoromethyl)cyclohexyl]-3-pyridyl]-2,5-
dimethyl-4-oxo-1H-pyridine-3-carboxamide

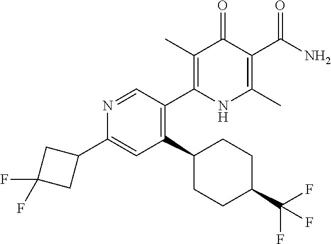 [SMILES: Cc1[nH]c(-c2cnc(C3CC(F)(F)C3)cc2[C@H]2CC[C@@H](C(F)(F)F)CC2)c(C)c(=O)c1C(N)=O]

6-[6-(3,3-difluorocyclobutyl)-4-((1s,4s)-4-
(trifluoromethyl)cyclohexyl)-3-pyridyl]-2,5-
dimethyl-4-oxo-1H-pyridine-3-carboxmaide/

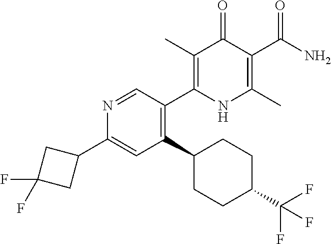 [SMILES: Cc1[nH]c(-c2cnc(C3CC(F)(F)C3)cc2[C@H]2CC[C@H](C(F)(F)F)CC2)c(C)c(=O)c1C(N)=O]

6-[6-(3,3-difluorocyclobutyl)-4-((1r,4r)-4-
(trifluoromethyl)cyclohexyl)-3-pyridyl]-2,5-
dimethyl-4-oxo-1H-pyridine-3-carboxamide

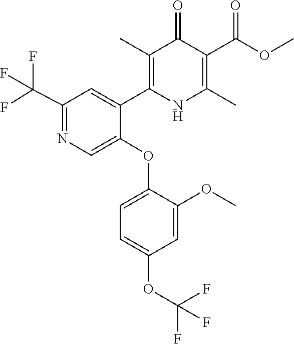 [SMILES: COC(=O)c1c(C)[nH]c(-c2cc(C(F)(F)F)ncc2Oc2ccc(OC(F)(F)F)cc2OC)c(C)c1=O]

methyl 6-[5-[2-methoxy-4-
(trifluoromethoxy)phenoxy]-2-(trifluoromethyl)-
4-pyridyl]-2,5-dimethyl-4-oxo-1H-pyridine-3-
carboxylate

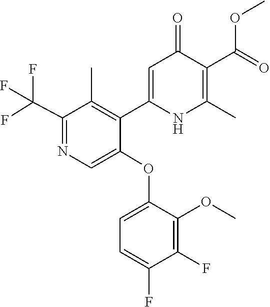 [SMILES: COC(=O)c1c(C)[nH]c(-c2c(Oc3ccc(F)c(F)c3OC)cnc(C(F)(F)F)c2C)cc1=O]

methyl 6-[5-(3,4-difluoro-2-methoxy-phenoxy)-3-
methyl-2-(trifluoromethyl)-4-pyridyl]-2-methyl-4-
oxo-1H-pyridine-3-carboxylate

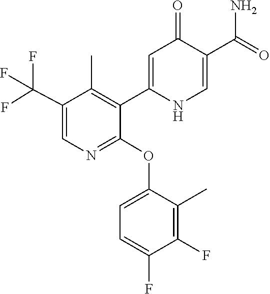 [SMILES: Cc1c(Oc2ncc(C(F)(F)F)c(C)c2-c2cc(=O)c(C(N)=O)c[nH]2)ccc(F)c1F]

6-[2-(3,4-difluoro-2-methyl-phenoxy)-4-methyl-
5-(trifluoromethyl)-3-pyridyl]-4-oxo-1H-pyridine-
3-carboxamide

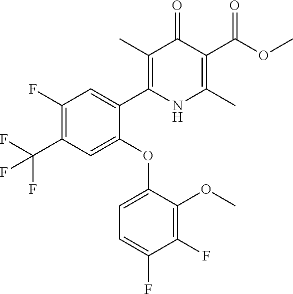 [SMILES: COC(=O)c1c(C)[nH]c(-c2cc(F)c(C(F)(F)F)cc2Oc2ccc(F)c(F)c2OC)c(C)c1=O]

methyl 6-[2-(3,4-difluoro-2-methoxy-phenoxy)-5-
fluoro-4-(trifluoromethyl)phenyl]-2,5-dimethyl-4-
oxo-1H-pyridine-3-carboxylate

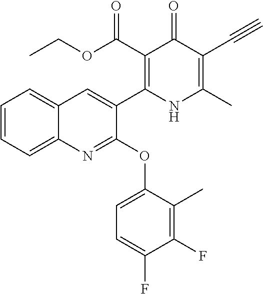 [SMILES: C#Cc1c(C)[nH]c(-c2cc3ccccc3nc2Oc2ccc(F)c(F)c2C)c(C(=O)OCC)c1=O]

ethyl 2-[2-(3,4-difluoro-2-methyl-phenoxy)-3-
quinolyl]-5-ethynyl-6-methyl-4-oxo-1H-pyridine-
3-carboxylate

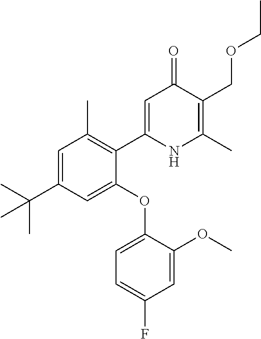 [SMILES: CCOCc1c(C)[nH]c(-c2c(C)cc(C(C)(C)C)cc2Oc2ccc(F)cc2OC)cc1=O]

6-[4-tert-butyl-2-(4-fluoro-2-methoxy-phenoxy)-
6-methyl-phenyl]-3-(ethoxymethyl)-2-methyl-1H-
pyridin-4-one

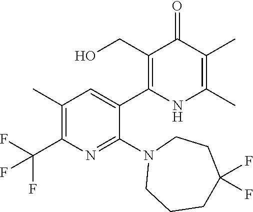 [SMILES: Cc1cc(-c2[nH]c(C)c(C)c(=O)c2CO)c(N2CCCC(F)(F)CC2)nc1C(F)(F)F]

2-[2-(4,4-difluoroazepan-1-yl)-5-methyl-6-
(trifluoromethyl)-3-pyridyl]-3-(hydroxymethyl)-
5,6-dimethyl-1H-pyridin-4-one

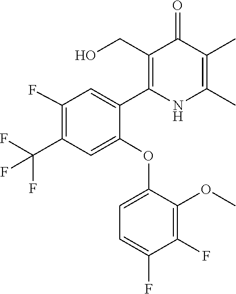 [SMILES: COc1c(Oc2cc(C(F)(F)F)c(F)cc2-c2[nH]c(C)c(C)c(=O)c2CO)ccc(F)c1F]

2-[2-(3,4-difluoro-2-methoxy-phenoxy)-5-fluoro-
4-(trifluoromethyl)phenyl]-3-(hydroxymethyl)-
5,6-dimethyl-1H-pyridin-4-one

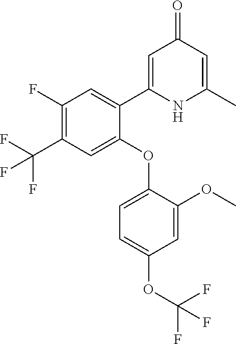 [SMILES: COc1cc(OC(F)(F)F)ccc1Oc1cc(C(F)(F)F)c(F)cc1-c1cc(=O)cc(C)[nH]1]

2-[5-fluoro-2-[2-methoxy-4-
(trifluoromethoxy)phenoxy]-4-
(trifluoromethyl)phenyl]-6-methyl-1H-pyridin-4-
one

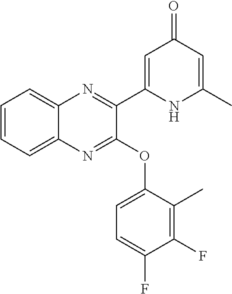 [SMILES: Cc1cc(=O)cc(-c2nc3ccccc3nc2Oc2ccc(F)c(F)c2C)[nH]1]

2-[3-(3,4-difluoro-2-methyl-phenoxy)quinoxalin-
2-yl]-6-methyl-1H-pyridin-4-one

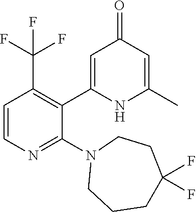 [SMILES: Cc1cc(=O)cc(-c2c(C(F)(F)F)ccnc2N2CCCC(F)(F)CC2)[nH]1]

2-[2-(4,4-difluoroazepan-1-yl)-4-
(trifluoromethyl)-3-pyridyl]-6-methyl-1H-pyridin-
4-one

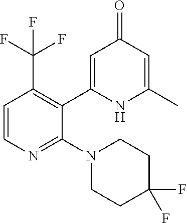 [SMILES: Cc1cc(=O)cc(-c2c(C(F)(F)F)ccnc2N2CCC(F)(F)CC2)[nH]1]

2-[2-(4,4-difluoro-1-piperidyl)-4-
(trifluoromethyl)-3-pyridyl]-6-methyl-1H-pyridin-
4-one

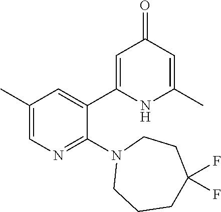 [SMILES: Cc1cnc(N2CCCC(F)(F)CC2)c(-c2cc(=O)cc(C)[nH]2)c1]

2-[2-(4,4-difluoroazepan-1-yl)-5-methyl-3-
pyridyl]-6-methyl-1H-pyridin-4-one

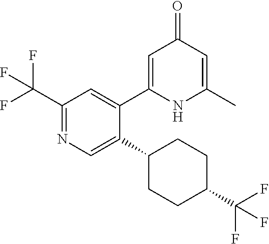 [SMILES: Cc1cc(=O)cc(-c2cc(C(F)(F)F)ncc2[C@H]2CC[C@@H](C(F)(F)F)CC2)[nH]1]

2-methyl-6-[2-(trifluoromethyl)-5-[4-
(trifluoromethyl)cyclohexyl]-4-pyridyl]-1H-
pyridin-4-one

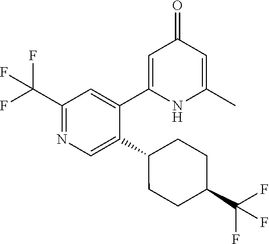 [SMILES: Cc1cc(=O)cc(-c2cc(C(F)(F)F)ncc2[C@H]2CC[C@H](C(F)(F)F)CC2)[nH]1]

2-methyl-6-[2-(trifluoromethyl)-5-((1r4r)-4-
(trifluoromethyl)cyclohexyl)-4-pyridyl]-1H-
pyridin-4-one

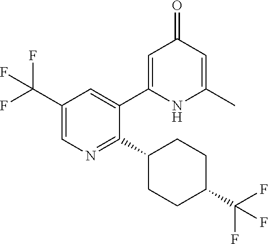 [SMILES: Cc1cc(=O)cc(-c2cc(C(F)(F)F)cnc2[C@H]2CC[C@@H](C(F)(F)F)CC2)[nH]1]

2-methyl-6-[2-(trifluoromethyl)-5-((1s,4s)-4-
(trifluoromethyl)cyclohexyl)-4-pyridyl]-1H-
pyridin-4-one

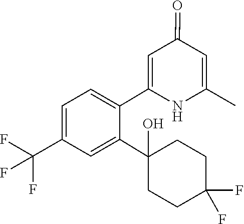 [SMILES: Cc1cc(=O)cc(-c2ccc(C(F)(F)F)cc2C2(O)CCC(F)(F)CC2)[nH]1]

2-[2-(4,4-difluoro-1-hydroxy-cyclohexyl)-4-
(trifluoromethyl)phenyl]-6-methyl-1H-pyridin-4-
one

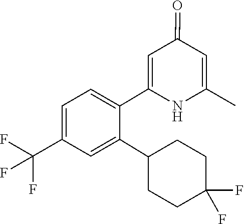 [SMILES: Cc1cc(=O)cc(-c2ccc(C(F)(F)F)cc2C2CCC(F)(F)CC2)[nH]1]

2-[2-(4,4-difluorocyclohexyl)-4-
(trifluoromethyl)phenyl]-6-methyl-1H-pyridin-4-
one

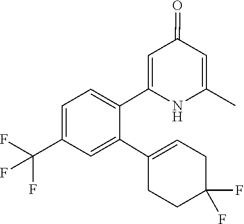 [SMILES: Cc1cc(=O)cc(-c2ccc(C(F)(F)F)cc2C2=CCC(F)(F)CC2)[nH]1]

2-[2-(4,4-difluorocyclohexen-1-yl)-4-
(trifluoromethyl)phenyl]-6-methyl-1H-pyridin-4-
one

In some embodiments, the invention relates to a compound selected from Table B, or a pharmaceutically acceptable salt thereof. In other embodiments, the invention relates to a compound selected from Table B, i.e., the compound in non-salt form.

TABLE B
[Compound Structures and Names.

| | |
|---|---|
| 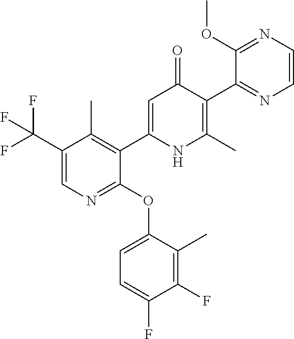 [SMILES: COc1nccnc1-c1c(C)[nH]c(-c2c(Oc3ccc(F)c(F)c3C)ncc(C(F)(F)F)c2C)cc1=O] | 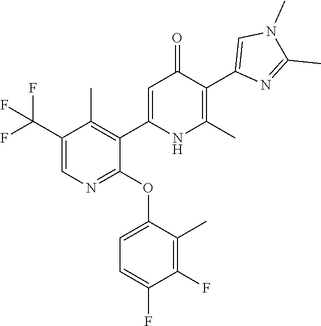 [SMILES: Cc1[nH]c(-c2c(Oc3ccc(F)c(F)c3C)ncc(C(F)(F)F)c2C)cc(=O)c1-c1cn(C)c(C)n1] |
| 6-[2-(3,4-difluoro-2-methyl-phenoxy)-4-methyl- | 6-[2-(3,4-difluoro-2-methyl-phenoxy)-4-methyl- |
| 5-(trifluoromethyl)-3-pyridyl]-3-(3- | 5-(trifluoromethyl)-3-pyridyl]-3-(1,2- |
| methoxypyrazin-2-yl)-2-methyl-1H-pyridin-4- | dimethylimidazol-4-yl)-2-methyl-1H-pyridin-4- |
| one | one |
| 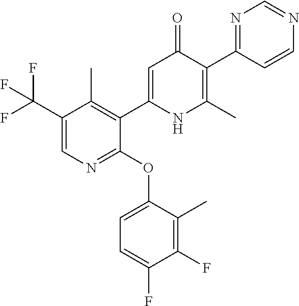 [SMILES: Cc1[nH]c(-c2c(Oc3ccc(F)c(F)c3C)ncc(C(F)(F)F)c2C)cc(=O)c1-c1ccncn1] | 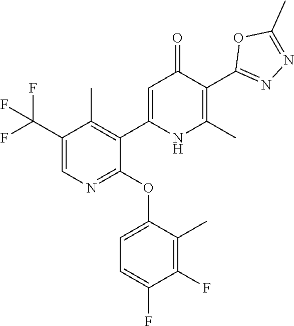 [SMILES: Cc1nnc(-c2c(C)[nH]c(-c3c(Oc4ccc(F)c(F)c4C)ncc(C(F)(F)F)c3C)cc2=O)o1] |
| 6-[2-(3,4-difluoro-2-methyl-phenoxy)-4-methyl- | 6-[2-(3,4-difluoro-2-methyl-phenoxy)-4-methyl- |
| 5-(trifluoromethyl)-3-pyridyl]-2-methyl-3- | 5-(trifluoromethyl)-3-pyridyl]-2-methyl-3-(5- |
| pyrimidin-4-yl-1H-pyridin-4-one | methyl-1,3,4-oxadiazol-2-yl)-1H-pyridin-4-one |
| 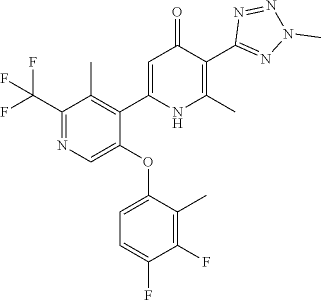 [SMILES: Cc1[nH]c(-c2c(Oc3ccc(F)c(F)c3C)cnc(C(F)(F)F)c2C)cc(=O)c1-c1nnn(C)n1] | 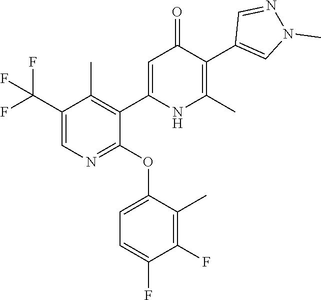 [SMILES: Cc1[nH]c(-c2c(Oc3ccc(F)c(F)c3C)ncc(C(F)(F)F)c2C)cc(=O)c1-c1cnn(C)c1] |
| 6-[5-(3,4-difluoro-2-methyl-phenoxy)-3-methyl- | 6-[2-(3,4-difluoro-2-methyl-phenoxy)-4-methyl- |
| 2-(trifluoromethyl)-4-pyridyl]-2-methyl-3-(2- | 5-(trifluoromethyl)-3-pyridyl]-2-methyl-3-(1- |
| methyltetrazol-5-yl)-1H-pyridin-4-one | methylpyrazol-4-yl)-1H-pyridin-4-one |
| 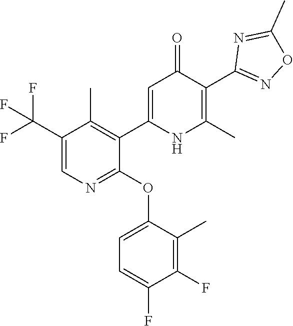 [SMILES: Cc1nc(-c2c(C)[nH]c(-c3c(Oc4ccc(F)c(F)c4C)ncc(C(F)(F)F)c3C)cc2=O)no1] | 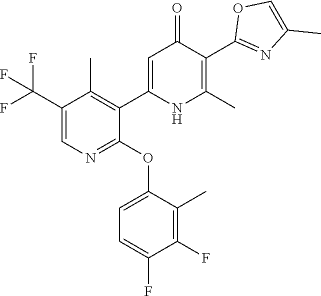 [SMILES: Cc1coc(-c2c(C)[nH]c(-c3c(Oc4ccc(F)c(F)c4C)ncc(C(F)(F)F)c3C)cc2=O)n1] |
| 6-[2-(3,4-difluoro-2-methyl-phenoxy)-4-methyl- | 6-[2-(3,4-difluoro-2-methyl-phenoxy)-4-methyl- |
| 5-(trifluoromethyl)-3-pyridyl]-2-methyl-3-(5- | 5-(trifluoromethyl)-3-pyridyl]-2-methyl-3-(4- |
| methyl-1,2,4-oxadiazol-3-yl)-1H-pyridin-4-one | methyloxazol-2-yl)-1H-pyridin-4-one |
| 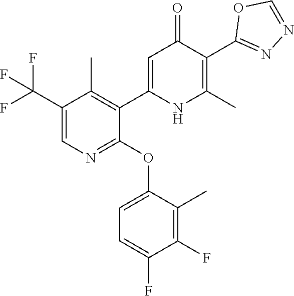 [SMILES: Cc1[nH]c(-c2c(Oc3ccc(F)c(F)c3C)ncc(C(F)(F)F)c2C)cc(=O)c1-c1nnco1] | 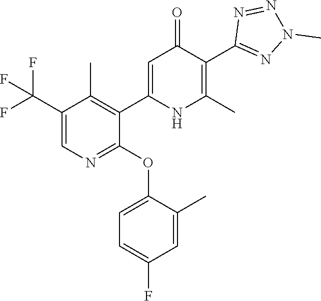 [SMILES: Cc1cc(F)ccc1Oc1ncc(C(F)(F)F)c(C)c1-c1cc(=O)c(-c2nnn(C)n2)c(C)[nH]1] |
| 6-[2-(3,4-difluoro-2-methyl-phenoxy)-4-methyl- | 6-[2-(4-fluoro-2-methyl-phenoxy)-4-methyl-5- |
| 5-(trifluoromethyl)-3-pyridyl]-2-methyl-3-(1,3,4- | (trifluoromethyl)-3-pyridyl]-2-methyl-3-(2- |
| oxadiazol-2-yl)-1H-pyridin-4-one | methyltetrazol-5-yl)-1H-pyridin-4-one |
| 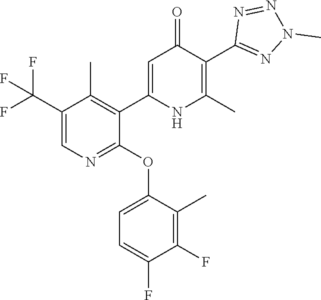 [SMILES: Cc1[nH]c(-c2c(Oc3ccc(F)c(F)c3C)ncc(C(F)(F)F)c2C)cc(=O)c1-c1nnn(C)n1] | 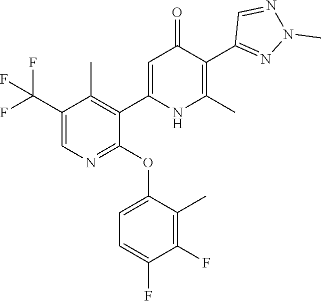 [SMILES: Cc1[nH]c(-c2c(Oc3ccc(F)c(F)c3C)ncc(C(F)(F)F)c2C)cc(=O)c1-c1cnn(C)n1] |
| 6-[2-(3,4-difluoro-2-methyl-phenoxy)-4-methyl- | 6-[2-(3,4-difluoro-2-methyl-phenoxy)-4-methyl- |
| 5-(trifluoromethyl)-3-pyridyl]-2-methyl-3-(2- | 5-(trifluoromethyl)-3-pyridyl]-2-methyl-3-(2- |
| methyltetrazol-5-yl)-1H-pyridin-4-one | methyltriazol-4-yl)-1H-pyridin-4-one |
| 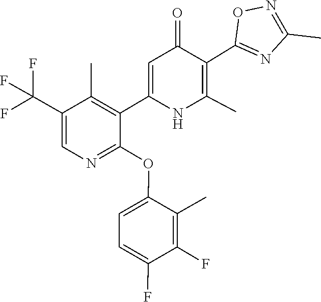 [SMILES: Cc1noc(-c2c(C)[nH]c(-c3c(Oc4ccc(F)c(F)c4C)ncc(C(F)(F)F)c3C)cc2=O)n1] | 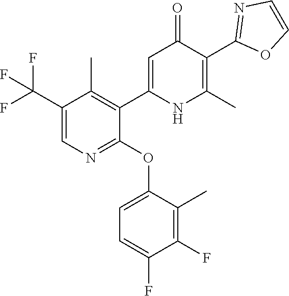 [SMILES: Cc1[nH]c(-c2c(Oc3ccc(F)c(F)c3C)ncc(C(F)(F)F)c2C)cc(=O)c1-c1ncco1] |
| 6-[2-(3,4-difluoro-2-methyl-phenoxy)-4-methyl- | 6-[2-(3,4-difluoro-2-methyl-phenoxy)-4-methyl- |
| 5-(trifluoromethyl)-3-pyridyl]-2-methyl-3-(3- | 5-(trifluoromethyl)-3-pyridyl]-2-methyl-3- |
| methyl-1,2,4-oxadiazol-5-yl)-1H-pyridin-4-one | oxazol-2-yl-1H-pyridin-4-one |
| 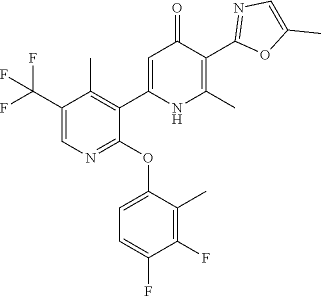 [SMILES: Cc1cnc(-c2c(C)[nH]c(-c3c(Oc4ccc(F)c(F)c4C)ncc(C(F)(F)F)c3C)cc2=O)o1] | 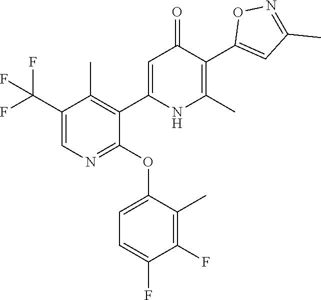 [SMILES: Cc1cc(-c2c(C)[nH]c(-c3c(Oc4ccc(F)c(F)c4C)ncc(C(F)(F)F)c3C)cc2=O)on1] |
| 6-[2-(3,4-difluoro-2-methyl-phenoxy)-4-methyl- | 6-[2-(3,4-difluoro-2-methyl-phenoxy)-4-methyl- |
| 5-(trifluoromethyl)-3-pyridyl]-2-methyl-3-(5- | 5-(trifluoromethyl)-3-pyridyl]-2-methyl-3-(3- |
| methyloxazol-2-yl)-1H-pyridin-4-one | methylisoxazol-5-yl)-1H-pyridin-4-one |
| 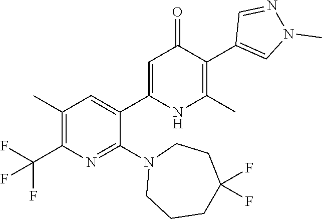 [SMILES: Cc1cc(-c2cc(=O)c(-c3cnn(C)c3)c(C)[nH]2)c(N2CCCC(F)(F)CC2)nc1C(F)(F)F] | 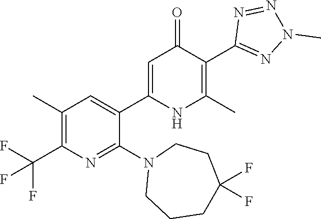 [SMILES: Cc1cc(-c2cc(=O)c(-c3nnn(C)n3)c(C)[nH]2)c(N2CCCC(F)(F)CC2)nc1C(F)(F)F] |
| 6-[2-(4,4-difluoroazepan-1-yl)-5-methyl-6- | 6-[2-(4,4-difluoroazepan-1-yl)-5-methyl-6- |
| (trifluoromethyl)-3-pyridyl]-2-methyl-3-(1- | (trifluoromethyl)-3-pyridyl]-2-methyl-3-(2- |
| mcthylpyrazol-4-yl)-1H-pyridin-4-onc | mcthyltetrazol-5-yl)-1H-pyridin-4-onc |
| 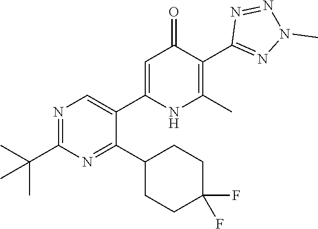 6-[2-tert-butyl-4-(4,4- difluorocyclohexyl)pyrimidin-5-yl]-2-methyl-3- (2-methyltetrazol-5-yl)-1H-pyridin-4-one | 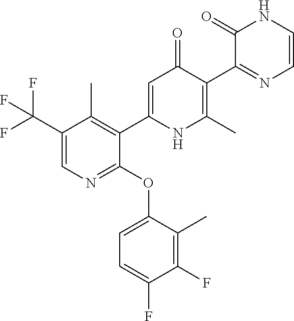 [SMILES: Cc1[nH]c(-c2c(Oc3ccc(F)c(F)c3C)ncc(C(F)(F)F)c2C)cc(=O)c1-c1ncc[nH]c1=O] |
| | 3-[6-[2-(3,4-difluoro-2-methyl-phenoxy)-4- |
| | methyl-5-(trifluoromethyl)-3-pyridyl]-2-methyl- |
| | 4-oxo-1H-pyridin-3-yl]-1H-pyrazin-2-one |
| 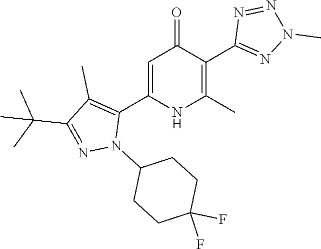 | 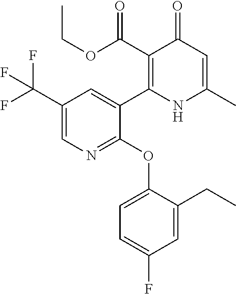 [SMILES: CCOC(=O)c1c(-c2cc(C(F)(F)F)cnc2Oc2ccc(F)cc2CC)[nH]c(C)cc1=O] |
| 6-[5-tert-butyl-2-(4,4-difluorocyclohexyl)-4- | ethyl 2-[2-(2-ethyl-4-fluoro-phenoxy)-5- |
| methyl-pyrazol-3-yl]-2-methyl-3-(2- | (trifluoromethyl)-3-pyridyl]-6-methyl-4-oxo-1H- |
| methyltetrazol-5-yl)-1H-pyridin-4-one | pyridine-3-carboxylate |
| 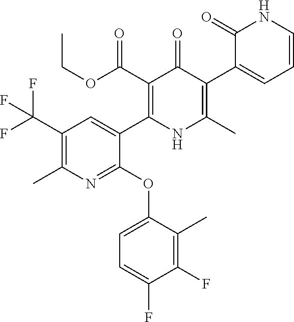 [SMILES: CCOC(=O)c1c(-c2cc(C(F)(F)F)c(C)nc2Oc2ccc(F)c(F)c2C)[nH]c(C)c(-c2ccc[nH]c2=O)c1=O] | 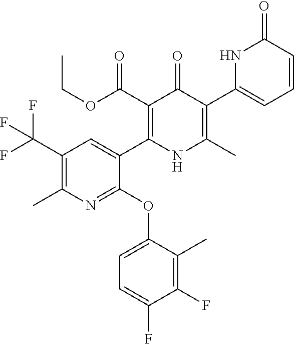 [SMILES: CCOC(=O)c1c(-c2cc(C(F)(F)F)c(C)nc2Oc2ccc(F)c(F)c2C)[nH]c(C)c(-c2cccc(=O)[nH]2)c1=O] |
| ethyl 2-[2-(3,4-difluoro-2-methyl-phenoxy)-6- | ethyl 2-[2-(3,4-difluoro-2-methyl-phenoxy)-6- |
| methyl-5-(trifluoromethyl)-3-pyridyl]-6-methyl- | methyl-5-(trifluoromethyl)-3-pyridyl]-6-methyl- |
| 4-oxo-5-(2-oxo-1H-pyridin-3-yl)-1H-pyridine-3- | 4-oxo-5-(6-oxo-1H-pyridin-2-yl)-1H-pyridine-3- |
| carboxylate | carboxylate |
| 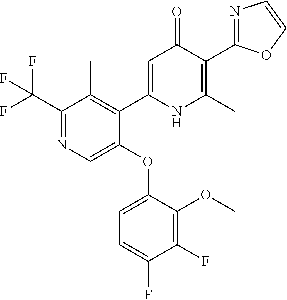 [SMILES: COc1c(Oc2cnc(C(F)(F)F)c(C)c2-c2cc(=O)c(-c3ncco3)c(C)[nH]2)ccc(F)c1F] | 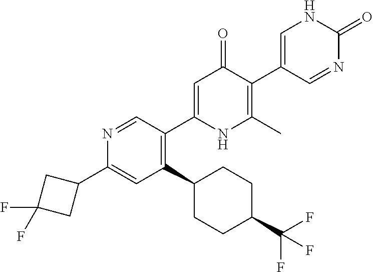 [SMILES: Cc1[nH]c(-c2cnc(C3CC(F)(F)C3)cc2[C@H]2CC[C@@H](C(F)(F)F)CC2)cc(=O)c1-c1cnc(=O)[nH]c1] |
| 6-[5-(3,4-difluoro-2-methoxy-phenoxy)-3- | 5-[6-[6-(3,3-difluorocyclobutyl)-4-[4- |
| methyl-2-(trifluoromethyl)-4-pyridyl]-2-methyl- | (trifluoromethyl)cyclohexyl]-3-pyridyl]-2- |
| 3-oxazol-2-yl-1H-pyridin-4-one | methyl-4-oxo-1H-pyridin-3-yl]-1H-pyrimidin-2- |
| | one |
| 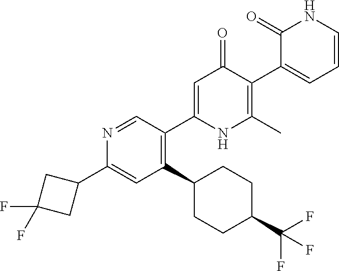 [SMILES: Cc1[nH]c(-c2cnc(C3CC(F)(F)C3)cc2[C@H]2CC[C@@H](C(F)(F)F)CC2)cc(=O)c1-c1ccc[nH]c1=O] | 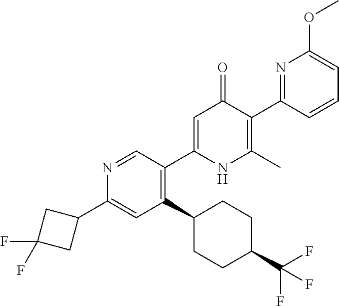 [SMILES: COc1cccc(-c2c(C)[nH]c(-c3cnc(C4CC(F)(F)C4)cc3[C@H]3CC[C@@H](C(F)(F)F)CC3)cc2=O)n1] |
| 6-[6-(3,3-difluorocyclobutyl)-4-[4- | 6-[6-(3,3-difluorocyclobutyl)-4-[4- |
| (trifluoromethyl)cyclohexyl]-3-pyridyl]-2- | (trifluoromethyl)cyclohexyl]-3-pyridyl]-3-(6- |
| methyl-3-(2-oxo-1H-pyridin-3-yl)-1H-pyridin-4- | methoxy-2-pyridyl)-2-methyl-1H-pyridin-4-one |
| one | |
| 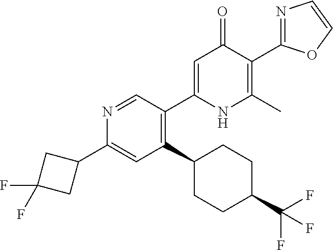 [SMILES: Cc1[nH]c(-c2cnc(C3CC(F)(F)C3)cc2[C@H]2CC[C@@H](C(F)(F)F)CC2)cc(=O)c1-c1ncco1] | 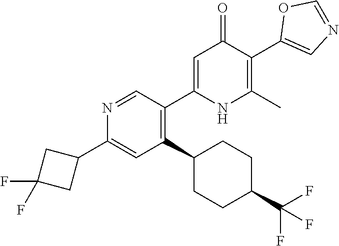 [SMILES: Cc1[nH]c(-c2cnc(C3CC(F)(F)C3)cc2[C@H]2CC[C@@H](C(F)(F)F)CC2)cc(=O)c1-c1cnco1] |
| 6-[6-(3,3-difluorocyclobutyl)-4-[4- | 6-[6-(3,3-difluorocyclobutyl)-4-[4- |
| (trifluoromethyl)cyclohexyl]-3-pyridyl]-2- | (trifluoromethyl)cyclohexyl]-3-pyridyl]-2- |
| methyl-3-oxazol-2-yl-1H-pyridin-4-one | methyl-3-oxazol-5-yl-1H-pyridin-4-one |
| 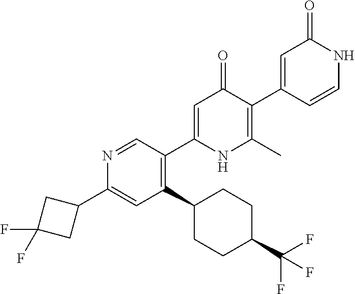 [SMILES: Cc1[nH]c(-c2cnc(C3CC(F)(F)C3)cc2[C@H]2CC[C@@H](C(F)(F)F)CC2)cc(=O)c1-c1cc[nH]c(=O)c1] | 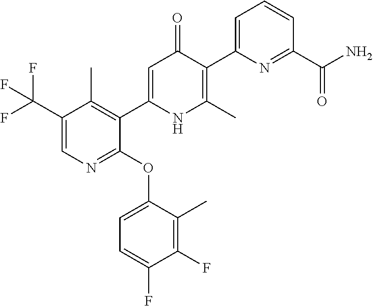 [SMILES: Cc1[nH]c(-c2c(Oc3ccc(F)c(F)c3C)ncc(C(F)(F)F)c2C)cc(=O)c1-c1cccc(C(N)=O)n1] |
| 6-[6-(3,3-difluorocyclobutyl)-4-[4- | 6-[6-[2-(3,4-difluoro-2-methyl-phenoxy)-4- |
| (trifluoromethyl)cyclohexyl]-3-pyridyl]-2- | methyl-5-(trifluoromethyl)-3-pyridyl]-2-methyl- |
| methyl-3-(2-oxo-1H-pyridin-4-yl)-1H-pyridin-4- | 4-oxo-1H-pyridin-3-yl\|pyridine-2-carboxamide |
| one | |
| 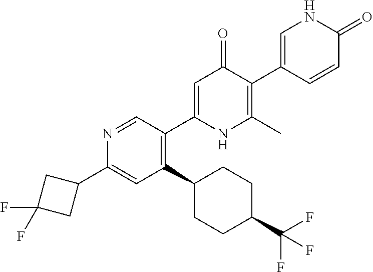 [SMILES: Cc1[nH]c(-c2cnc(C3CC(F)(F)C3)cc2[C@H]2CC[C@@H](C(F)(F)F)CC2)cc(=O)c1-c1ccc(=O)[nH]c1] | 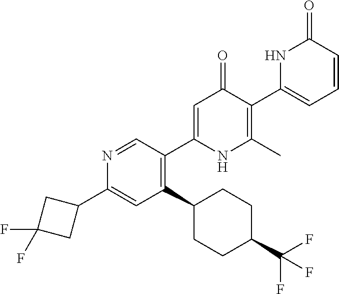 [SMILES: Cc1[nH]c(-c2cnc(C3CC(F)(F)C3)cc2[C@H]2CC[C@@H](C(F)(F)F)CC2)cc(=O)c1-c1cccc(=O)[nH]1] |
| 6-[6-(3,3-difluorocyclobutyl)-4-[4- | 6-[6-(3,3-difluorocyclobutyl)-4-[4- |
| (trifluoromethyl)cyclohexyl]-3-pyridyl]-2- | (trifluoromethyl)cyclohexyl]-3-pyridyl]-2- |
| methyl-3-(6-oxo-1H-pyridin-3-yl)-1H-pyridin-4- | methyl-3-(6-oxo-1H-pyridin-2-yl)-1H-pyridin-4- |
| one | one |
| 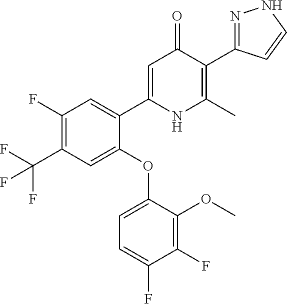 [SMILES: COc1c(Oc2cc(C(F)(F)F)c(F)cc2-c2cc(=O)c(-c3cc[nH]n3)c(C)[nH]2)ccc(F)c1F] | 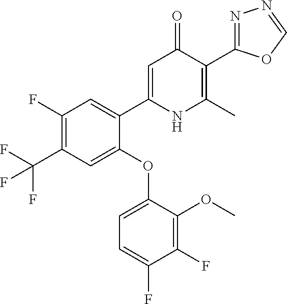 [SMILES: COc1c(Oc2cc(C(F)(F)F)c(F)cc2-c2cc(=O)c(-c3nnco3)c(C)[nH]2)ccc(F)c1F] |
| 6-[2-(3,4-difluoro-2-methoxy-phenoxy)-5- | .6-[2-(3,4-difluoro-2-methoxy-phenoxy)-5- |
| fluoro-4-(trifluoromethyl)phenyl]-2-methyl-3- | fluoro-4-(trifluoromethyl)phenyl]-2-methyl-3- |
| (1H-pyrazol-3-yl)-1H-pyridin-4-one | (1,3,4-oxadiazol-2-yl)-1H-pyridin-4-one |
| 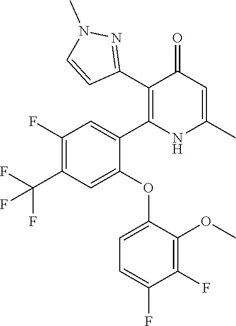 [SMILES: COc1c(Oc2cc(C(F)(F)F)c(F)cc2-c2[nH]c(C)cc(=O)c2-c2ccn(C)n2)ccc(F)c1F] | 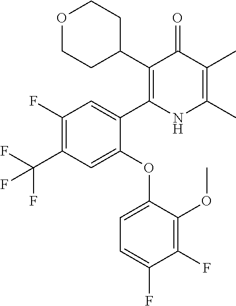 [SMILES: COc1c(Oc2cc(C(F)(F)F)c(F)cc2-c2[nH]c(C)c(C)c(=O)c2C2CCOCC2)ccc(F)c1F] |
| 2-[2-(3,4-difluoro-2-methoxy-phenoxy)-5- | 2-[2-(3,4-difluoro-2-methoxy-phenoxy)-5- |
| fluoro-4-(trifluoromethyl)phenyl]-6-methyl-3-(1- | fluoro-4-(trifluoromethyl)phenyl]-5,6-dimethyl- |
| methylpyrazol-3-yl)-1H-pyridin-4-one | 3-tetrahydropyran-4-yl-1H-pyridin-4-one |
| 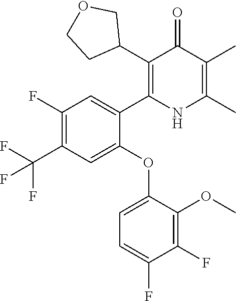 [SMILES: COc1c(Oc2cc(C(F)(F)F)c(F)cc2-c2[nH]c(C)c(C)c(=O)c2C2CCOC2)ccc(F)c1F] | 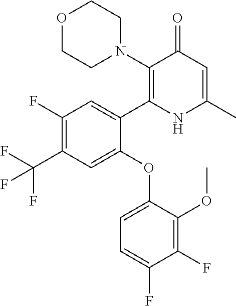 [SMILES: COc1c(Oc2cc(C(F)(F)F)c(F)cc2-c2[nH]c(C)cc(=O)c2N2CCOCC2)ccc(F)c1F] |
| 2-[2-(3,4-difluoro-2-methoxy-phenoxy)-5- | 2-[2-(3,4-difluoro-2-methoxy-phenoxy)-5- |
| fluoro-4-(trifluoromethyl)phenyl]-5,6-dimethyl- | fluoro-4-(trifluoromethyl)phenyl]-6-methyl-3- |
| 3-tetrahydrofuran-3-yl-1H-pyridin-4-one | morpholino-1H-pyridin-4-one |
| 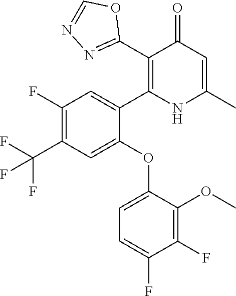 [SMILES: COc1c(Oc2cc(C(F)(F)F)c(F)cc2-c2[nH]c(C)cc(=O)c2-c2nnco2)ccc(F)c1F] | 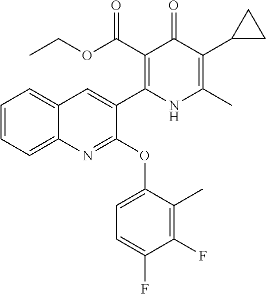 [SMILES: CCOC(=O)c1c(-c2cc3ccccc3nc2Oc2ccc(F)c(F)c2C)[nH]c(C)c(C2CC2)c1=O] |
| 2-[2-(3,4-difluoro-2-methoxy-phenoxy)-5- | ethyl 5-cyclopropyl-2-[2-(3,4-difluoro-2-methyl- |
| fluoro-4-(trifluoromethyl)phenyl]-6-methyl-3- | phenoxy)-3-quinolyl]-6-methyl-4-oxo-1H- |
| (1,3,4-oxadiazol-2-yl)-1H-pyridin-4-one | pyridine-3-carboxylate |
| 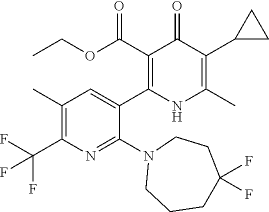 [SMILES: CCOC(=O)c1c(-c2cc(C)c(C(F)(F)F)nc2N2CCCC(F)(F)CC2)[nH]c(C)c(C2CC2)c1=O] ethyl 5-cyclopropyl-2-[2-(4,4-difluoroazepan-1- yl)-5-methyl-6-(trifluoromethyl)-3-pyridyl]-6- methyl-4-oxo-1H-pyridine-3-carboxylate | 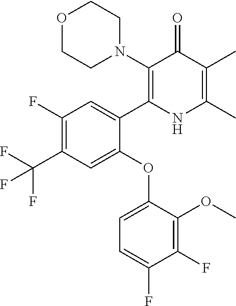 [SMILES: COc1c(Oc2cc(C(F)(F)F)c(F)cc2-c2[nH]c(C)c(C)c(=O)c2N2CCOCC2)ccc(F)c1F] |
| | 2-[2-(3,4-difluoro-2-methoxy-phenoxy)-5- |
| | fluoro-4-(trifluoromethyl)phenyl]-5,6-dimethyl- |
| | 3-morpholino-1H-pyridin-4-one |
| 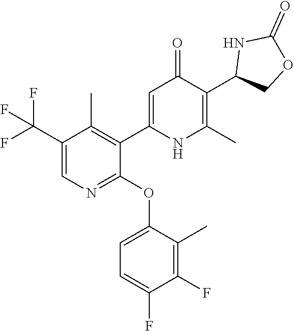 [SMILES: Cc1[nH]c(-c2c(Oc3ccc(F)c(F)c3C)ncc(C(F)(F)F)c2C)cc(=O)c1[C@@H]1COC(=O)N1] | 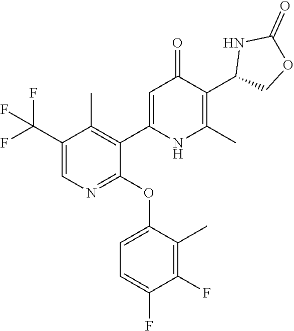 [SMILES: Cc1[nH]c(-c2c(Oc3ccc(F)c(F)c3C)ncc(C(F)(F)F)c2C)cc(=O)c1[C@H]1COC(=O)N1] |
| (R)-4-[6-[2-(3,4-difluoro-2-methyl-phenoxy)-4- | (S)-4-[6-[2-(3,4-difluoro-2-methyl-phenoxy)-4- |
| methyl-5-(trifluoromethyl)-3-pyridyl]-2-methyl- | methyl-5-(trifluoromethyl)-3-pyridyl]-2-methyl- |
| 4-oxo-1H-pyridin-3-yl]oxazolidin-2-one | 4-oxo-1H-pyridin-3-yl]oxazolidin-2-one |
| 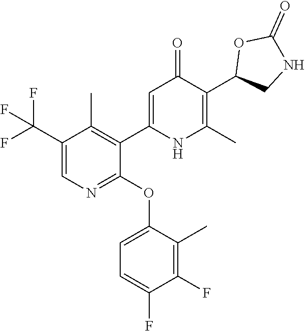 [SMILES: Cc1[nH]c(-c2c(Oc3ccc(F)c(F)c3C)ncc(C(F)(F)F)c2C)cc(=O)c1[C@@H]1CNC(=O)O1] | 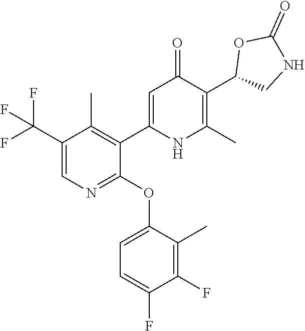 [SMILES: Cc1[nH]c(-c2c(Oc3ccc(F)c(F)c3C)ncc(C(F)(F)F)c2C)cc(=O)c1[C@H]1CNC(=O)O1] |
| (R)-5-[6-[2-(3,4-difluoro-2-methyl-phenoxy)-4- | (S)-5-[6-[2-(3,4-difluoro-2-methyl-phenoxy)-4- |
| methyl-5-(trifluoromethyl)-3-pyridyl]-2-methyl- | methyl-5-(trifluoromethyl)-3-pyridyl]-2-methyl- |
| 4-oxo-1H-pyridin-3-yl]oxazolidin-2-one | 4-oxo-1H-pyridin-3-yl]oxazolidin-2-one |
| 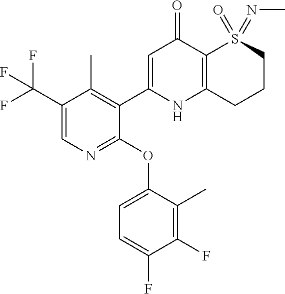 [SMILES: CN=[S@]1(=O)CCCc2[nH]c(-c3c(Oc4ccc(F)c(F)c4C)ncc(C(F)(F)F)c3C)cc(=O)c21] | 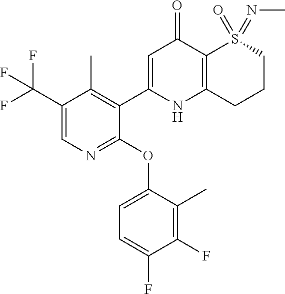 [SMILES: CN=[S@@]1(=O)CCCc2[nH]c(-c3c(Oc4ccc(F)c(F)c4C)ncc(C(F)(F)F)c3C)cc(=O)c21] |
| (S)-6-(2-(3,4-difluoro-2-methylphenoxy)-4- | (R)-6-(2-(3,4-difluoro-2-methylphenoxy)-4- |
| methyl-5-(trifluoromethyl)pyridin-3-yl)-1- | methyl-5-(trifluoromethyl)pyridin-3-yl)-1- |
| (methylimino)-1,3,4,5-tetrahydro-124- | (methylimino)-1,3,4,5-tetrahydro-124- |
| thiopyrano[3,2-b]pyridin-8(2H)-one 1-oxide | thiopyrano[3,2-b]pyridin-8(2H)-one 1-oxide |
| 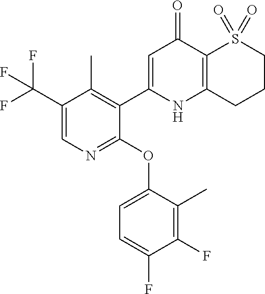 [SMILES: Cc1c(Oc2ncc(C(F)(F)F)c(C)c2-c2cc(=O)c3c([nH]2)CCCS3(=O)=O)ccc(F)c1F] | 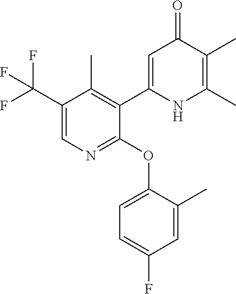 [SMILES: Cc1cc(F)ccc1Oc1ncc(C(F)(F)F)c(C)c1-c1cc(=O)c(C)c(C)[nH]1] |
| 6-(2-(3,4-difluoro-2-methylphenoxy)-4-methyl- | 6-(3-(4-fluoro-2-methylphenoxy)-5-methyl-6- |
| 5-(trifluoromethyl)pyridin-3-yl)-3,4-dihydro-2H- | (trifluoromethyl)pyridazin-4-yl)-2,3- |
| thiopyrano[3,2-b]pyridin-8(5H)-one 1,1-dioxide | dimethylpyridin-4(1H)-one |
| 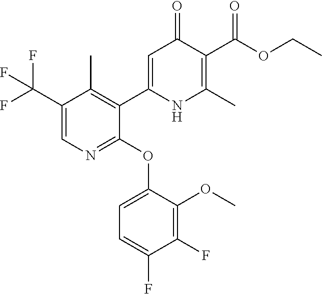 [SMILES: CCOC(=O)c1c(C)[nH]c(-c2c(Oc3ccc(F)c(F)c3OC)ncc(C(F)(F)F)c2C)cc1=O] | 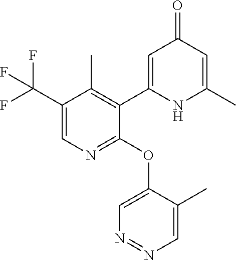 [SMILES: Cc1cc(=O)cc(-c2c(Oc3cnncc3C)ncc(C(F)(F)F)c2C)[nH]1] |
| ethyl 6-(3-(3,4-difluoro-2-methoxyphenoxy)-5- | 4',6-dimethyl-2'-((5-methylpyridazin-4-yl)oxy)- |
| methyl-6-(trifluoromethyl)pyridazin-4-yl)-2- | 5'-(trifluoromethyl)-[2,3'-bipyridin]-4(1H)-one |
| methyl-4-oxo-1,4-dihydropyridine-3-carboxylate | |
| 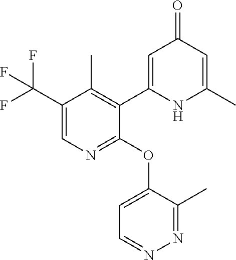 [SMILES: Cc1cc(=O)cc(-c2c(Oc3ccnnc3C)ncc(C(F)(F)F)c2C)[nH]1] | 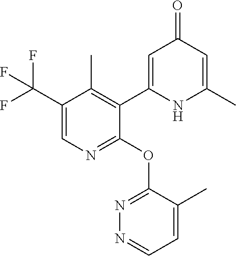 [SMILES: Cc1cc(=O)cc(-c2c(Oc3nnccc3C)ncc(C(F)(F)F)c2C)[nH]1] |
| 4',6-dimethyl-2'-((3-methylpyridazin-4-yl)oxy)- | 4',6-dimethyl-2'-((4-methylpyridazin-3-yl)oxy)- |
| 5'-(trifluoromethyl)-[2,3'-bipyridin]-4(1H)-one | 5'-(trifluoromethyl)-[2,3'-bipyridin]-4(1H)-one |
| 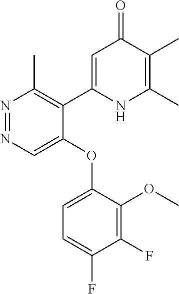 [SMILES: COc1c(Oc2cnnc(C)c2-c2cc(=O)c(C)c(C)[nH]2)ccc(F)c1F] | 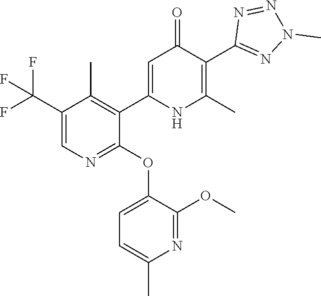 [SMILES: COc1nc(C)ccc1Oc1ncc(C(F)(F)F)c(C)c1-c1cc(=O)c(-c2nnn(C)n2)c(C)[nH]1] |
| 6-(5-(3,4-difluoro-2-methoxyphenoxy)-3- | 6-(3-((2-methoxy-6-methylpyridin-3-yl)oxy)-5- |
| methylpyridazin-4-yl)-2,3-dimethylpyridin- | methyl-6-(trifluoromethyl)pyridazin-4-yl)-2- |
| 4(1H)-one | methyl-3-(2-methyl-2H-tetrazol-5-yl)pyridin- |
| | 4(1H)-one |
| 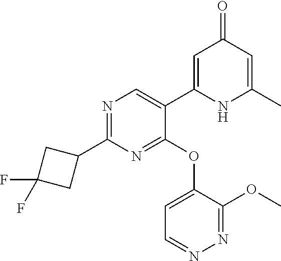 [SMILES: COc1nnccc1Oc1nc(C2CC(F)(F)C2)ncc1-c1cc(=O)cc(C)[nH]1] | 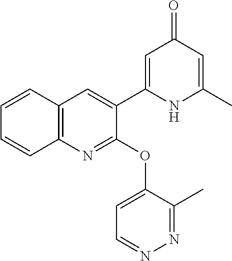 [SMILES: Cc1cc(=O)cc(-c2cc3ccccc3nc2Oc2ccnnc2C)[nH]1] |
| 2-(2-(3,3-difluorocyclobutyl)-4-((3- | 2-methyl-6-(2-((3-methylpyridazin-4- |
| methoxypyridazin-4-yl)oxy)pyrimidin-5-yl)-6- | yl)oxy)quinolin-3-yl)pyridin-4(1H)-one |
| methylpyridin-4(1H)-one | |
| 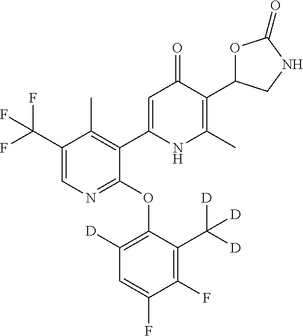 [SMILES: [2H]c1cc(F)c(F)c(C([2H])([2H])[2H])c1Oc1ncc(C(F)(F)F)c(C)c1-c1cc(=O)c(C2CNC(=O)O2)c(C)[nH]1] | 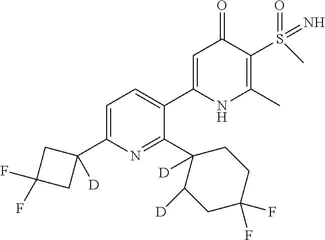 [SMILES: [2H]C1CC(F)(F)CCC1([2H])c1nc(C2([2H])CC(F)(F)C2)ccc1-c1cc(=O)c(S(C)(=N)=O)c(C)[nH]1] |
| 5-(2'-(3,4-difluoro-2-(methyl-d3)phenoxy-6-d)- | 6'-(3,3-difluorocyclobutyl-1-d)-2'-(4,4- |
| 4',6-dimethyl-4-oxo-5'-(trifluoromethyl)-1,4- | difluorocyclohexyl-1,2-d2)-6-methyl-5-(S- |
| dihydro-[2,3'-bipyridin]-5-yl)oxazolidin-2-one | methylsulfonimidoyl)-[2,3'-bipyridin]-4(1H)-one |
| 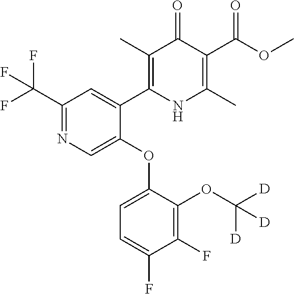 [SMILES: [2H]C([2H])([2H])Oc1c(Oc2cnc(C(F)(F)F)cc2-c2[nH]c(C)c(C(=O)OC)c(=O)c2C)ccc(F)c1F] | 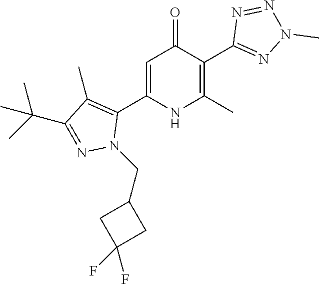 [SMILES: Cc1[nH]c(-c2c(C)c(C(C)(C)C)nn2CC2CC(F)(F)C2)cc(=O)c1-c1nnn(C)n1] |
| mcthyl 5'-(3,4-difluoro-2-(mcthoxy- | 6-(3-(tert-butyl)-1-((3,3- |
| d3)phenoxy)-3,6-dimethyl-4-oxo-2'- | difluorocyclobutyl)methyl)-4-methyl-1H- |
| (trifluoromethyl)-1,4-dihydro-[2,4'-bipyridine]- | pyrazol-5-yl)-2-methyl-3-(2-methyl-2H-tetrazol- |
| 5-carboxylate | 5-yl)pyridin-4(1H)-one |
| 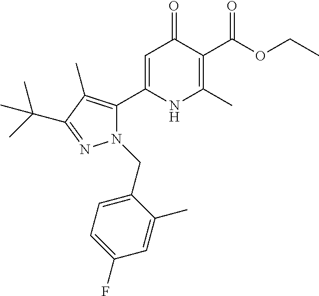 [SMILES: CCOC(=O)c1c(C)[nH]c(-c2c(C)c(C(C)(C)C)nn2Cc2ccc(F)cc2C)cc1=O] | 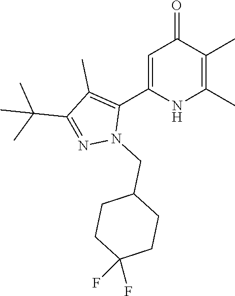 [SMILES: Cc1[nH]c(-c2c(C)c(C(C)(C)C)nn2CC2CCC(F)(F)CC2)cc(=O)c1C] |
| ethyl 6-(3-(tert-butyl)-1-(4-fluoro-2- | 6-(3-(tert-butyl)-1-((4,4- |
| methylbenzyl)-4-methyl-1H-pyrazol-5-yl)-2- | difluorocyclohexyl)methyl)-4-methyl-1H- |
| methyl-4-oxo-1,4-dihydropyridine-3-carboxylate | pyrazol-5-yl)-2,3-dimethylpyridin-4(1H)-one |

In some embodiments, the invention relates to a compound of formula

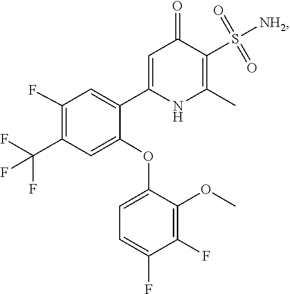 [SMILES: COc1c(Oc2cc(C(F)(F)F)c(F)cc2-c2cc(=O)c(S(N)(=O)=O)c(C)[nH]2)ccc(F)c1F]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

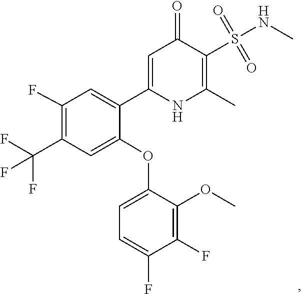 [SMILES: CNS(=O)(=O)c1c(C)[nH]c(-c2cc(F)c(C(F)(F)F)cc2Oc2ccc(F)c(F)c2OC)cc1=O]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

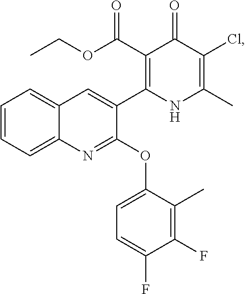 [SMILES: CCOC(=O)c1c(-c2cc3ccccc3nc2Oc2ccc(F)c(F)c2C)[nH]c(C)c(Cl)c1=O]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

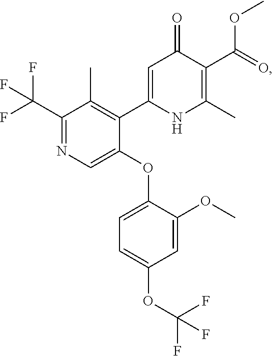 [SMILES: COC(=O)c1c(C)[nH]c(-c2c(Oc3ccc(OC(F)(F)F)cc3OC)cnc(C(F)(F)F)c2C)cc1=O]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

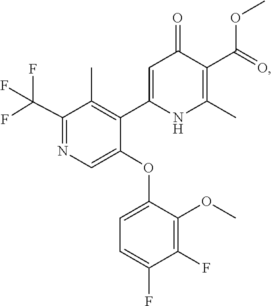 [SMILES: COC(=O)c1c(C)[nH]c(-c2c(Oc3ccc(F)c(F)c3OC)cnc(C(F)(F)F)c2C)cc1=O]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

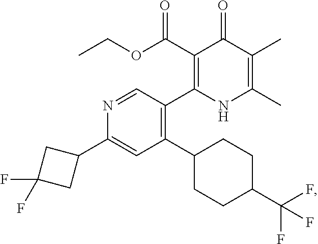 [SMILES: CCOC(=O)c1c(-c2cnc(C3CC(F)(F)C3)cc2C2CCC(C(F)(F)F)CC2)[nH]c(C)c(C)c1=O]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

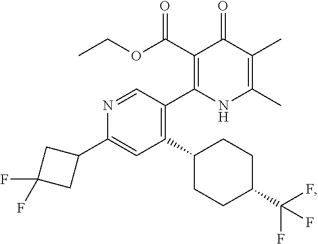 [SMILES: CCOC(=O)c1c(-c2cnc(C3CC(F)(F)C3)cc2[C@H]2CC[C@@H](C(F)(F)F)CC2)[nH]c(C)c(C)c1=O]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

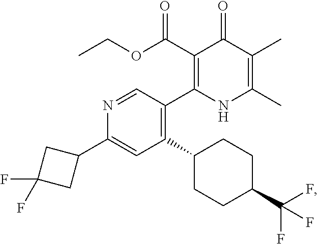 [SMILES: CCOC(=O)c1c(-c2cnc(C3CC(F)(F)C3)cc2[C@H]2CC[C@H](C(F)(F)F)CC2)[nH]c(C)c(C)c1=O]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

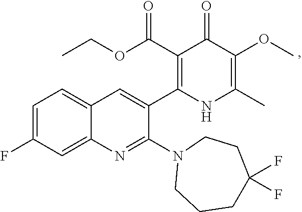 [SMILES: CCOC(=O)c1c(-c2cc3ccc(F)cc3nc2N2CCCC(F)(F)CC2)[nH]c(C)c(OC)c1=O]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

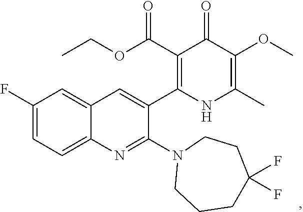 [SMILES: CCOC(=O)c1c(-c2cc3cc(F)ccc3nc2N2CCCC(F)(F)CC2)[nH]c(C)c(OC)c1=O]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

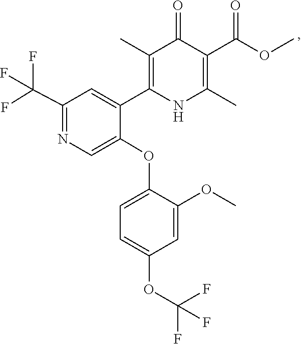 [SMILES: COC(=O)c1c(C)[nH]c(-c2cc(C(F)(F)F)ncc2Oc2ccc(OC(F)(F)F)cc2OC)c(C)c1=O]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

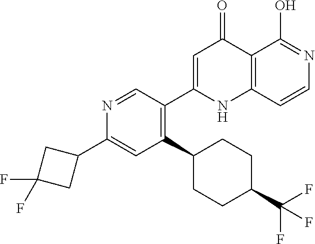 [SMILES: O=c1cc(-c2cnc(C3CC(F)(F)C3)cc2[C@H]2CC[C@@H](C(F)(F)F)CC2)[nH]c2ccnc(O)c12]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

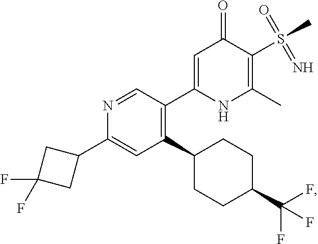 [SMILES: Cc1[nH]c(-c2cnc(C3CC(F)(F)C3)cc2[C@H]2CC[C@@H](C(F)(F)F)CC2)cc(=O)c1[S@@](C)(=N)=O]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

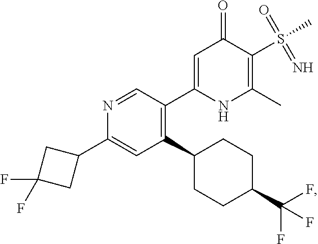 [SMILES: Cc1[nH]c(-c2cnc(C3CC(F)(F)C3)cc2[C@H]2CC[C@@H](C(F)(F)F)CC2)cc(=O)c1[S@](C)(=N)=O]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

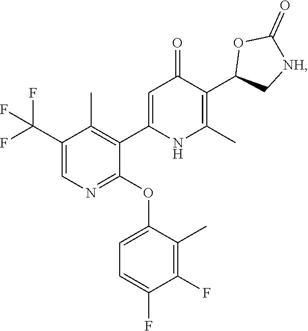 [SMILES: Cc1[nH]c(-c2c(Oc3ccc(F)c(F)c3C)ncc(C(F)(F)F)c2C)cc(=O)c1[C@@H]1CNC(=O)O1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

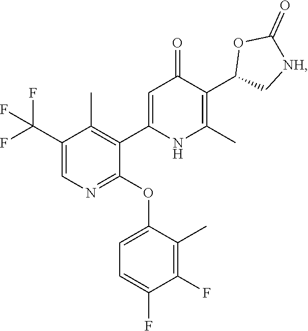 [SMILES: Cc1[nH]c(-c2c(Oc3ccc(F)c(F)c3C)ncc(C(F)(F)F)c2C)cc(=O)c1[C@H]1CNC(=O)O1]

or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound selected from

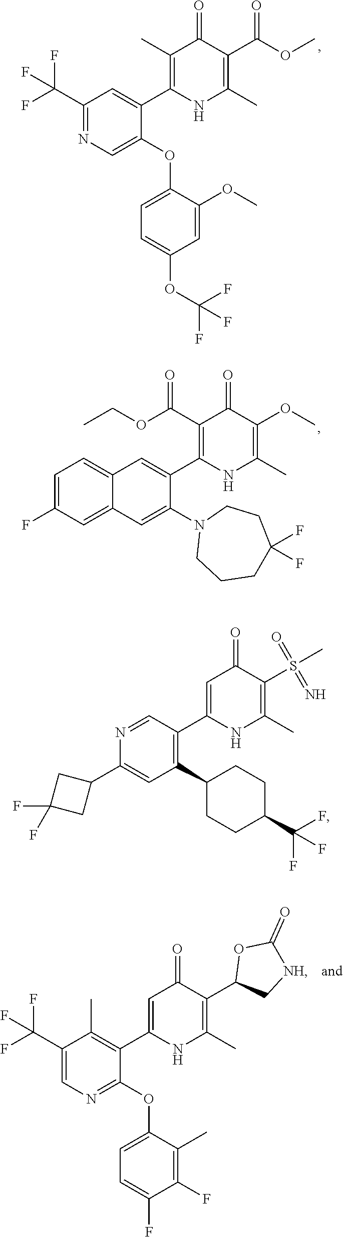

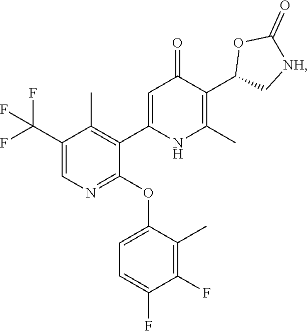 [SMILES: Cc1[nH]c(-c2c(Oc3ccc(F)c(F)c3C)ncc(C(F)(F)F)c2C)cc(=O)c1[C@H]1CNC(=O)O1]

or a pharmaceutically acceptable salt thereof. In other embodiments, the invention relates to the foregoing compound in non-salt form. Such compound is considered to be a “compound of the invention,” as that term is used herein.

### Salts, Compositions, Uses, Formulation, Administration and Additional Agents

### Pharmaceutically Acceptable Salts and Compositions

As discussed herein, the invention provides compounds, and pharmaceutically acceptable salts thereof, that are inhibitors of voltage-gated sodium channels, and thus the present compounds, and pharmaceutically acceptable salts thereof, are useful for the treatment of diseases, disorders, and conditions including, but not limited to chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, or cardiac arrhythmia. Accordingly, in another aspect of the invention, pharmaceutical compositions are provided, wherein these compositions comprise a compound as described herein, or a pharmaceutically acceptable salt thereof, and optionally comprise a pharmaceutically acceptable carrier, adjuvant or vehicle. In certain embodiments, these compositions optionally further comprise one or more additional therapeutic agents. In some embodiments, the additional therapeutic agent is a sodium channel inhibitor.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” of a compound of this invention includes any non-toxic salt that, upon administration to a recipient, is capable of providing, either directly or indirectly, a compound of this invention or an inhibitorily active metabolite or residue thereof. The salt may be in pure form, in a mixture (e.g., solution, suspension, or colloid) with one or more other substances, or in the form of a hydrate, solvate, or co-crystal. As used herein, the term “inhibitorily active metabolite or residue thereof” means that a metabolite or residue thereof is also an inhibitor of a voltage-gated sodium channel.

Pharmaceutically acceptable salts are well known in the art. For example, S. M. Berge, et al. describe pharmaceutically acceptable salts in detail in J Pharmaceutical Sciences, 1977, 66, 1-19, incorporated herein by reference. Pharmaceutically acceptable salts of the compound of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As described herein, the pharmaceutically acceptable compositions of the invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In another aspect, the invention features a pharmaceutical composition comprising a compound of the invention, or a pharmaceutically acceptable salt thereof, and a pharmaceutically acceptable carrier.

In another aspect, the invention features a pharmaceutical composition comprising a therapeutically effective amount of a compound, or a pharmaceutically acceptable salt thereof, and one or more pharmaceutically acceptable carriers or vehicles.

### Uses of Compounds and Pharmaceutically Acceptable Salts and Compositions

In another aspect, the invention features a method of inhibiting a voltage-gated sodium channel in a subject comprising administering to the subject a compound of the invention or a pharmaceutically acceptable salt thereof or a pharmaceutical composition thereof. In another aspect, the voltage-gated sodium channel is Na_{V}1.8.

In yet another aspect, the invention features a method of treating or lessening the severity in a subject of chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, or cardiac arrhythmia comprising administering an effective amount of a compound of the invention, a pharmaceutically acceptable salt thereof or a pharmaceutical composition thereof.

In yet another aspect, the invention features a method of treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia comprising administering an effective amount of a compound of the invention, a pharmaceutically acceptable salt thereof or a pharmaceutical composition thereof.

In yet another aspect, the invention features a method of treating or lessening the severity in a subject of gut pain, wherein gut pain comprises inflammatory bowel disease pain, Crohn's disease pain, irritable bowel syndrome, endometriosis, polycyctic ovarian disease, salpingitis, cervicitis or interstitial cystitis pain wherein said method comprises administering an effective amount of a compound of the invention, a pharmaceutically acceptable salt thereof or a pharmaceutical composition thereof.

In yet another aspect, the invention features a method of treating or lessening the severity in a subject of neuropathic pain comprising administering an effective amount of a compound of the invention, a pharmaceutically acceptable salt thereof or a pharmaceutical composition thereof. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In yet another aspect, the invention features a method of treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic cephalalgia wherein said method comprises administering an effective amount of a compound of the invention, a pharmaceutically acceptable salt thereof or a pharmaceutical composition thereof.

In yet another aspect, the invention features a method of treating or lessening the severity in a subject of musculoskeletal pain comprising administering an effective amount of a compound of the invention, a pharmaceutically acceptable salt thereof or a pharmaceutical composition thereof. In some aspects, the musculoskeletal pain comprises osteoarthritis pain.

In yet another aspect, the invention features a method of treating or lessening the severity in a subject of musculoskeletal pain, wherein musculoskeletal pain comprises osteoarthritis pain, back pain, cold pain, burn pain or dental pain wherein said method comprises administering an effective amount of a compound of the invention, a pharmaceutically acceptable salt thereof or a pharmaceutical composition thereof.

In yet another aspect, the invention features a method of treating or lessening the severity in a subject of inflammatory pain, wherein inflammatory pain comprises rheumatoid arthritis pain, ankylosing spondylitis or vulvodynia wherein said method comprises administering an effective amount of a compound of the invention, a pharmaceutically acceptable salt thereof or a pharmaceutical composition thereof.

In yet another aspect, the invention features a method of treating or lessening the severity in a subject of inflammatory pain, wherein inflammatory pain comprises rheumatoid arthritis pain wherein said method comprises administering an effective amount of a compound of the invention, a pharmaceutically acceptable salt thereof or a pharmaceutical composition thereof.

In yet another aspect, the invention features a method of treating or lessening the severity in a subject of idiopathic pain, wherein idiopathic pain comprises fibromyalgia pain wherein said method comprises administering an effective amount of a compound of the invention, a pharmaceutically acceptable salt thereof or a pharmaceutical composition thereof.

In yet another aspect, the invention features a method of treating or lessening the severity in a subject of idiopathic pain, wherein idiopathic pain comprises reflex sympathetic dystrophy pain, wherein said method comprises administering an effective amount of a compound of the invention, a pharmaceutically acceptable salt thereof or a pharmaceutical composition thereof.

In yet another aspect, the invention features a method of treating or lessening the severity in a subject of pathological cough wherein said method comprises administering an effective amount of a compound of the invention, a pharmaceutically acceptable salt thereof or a pharmaceutical composition thereof.

In yet another aspect, the invention features a method of treating or lessening the severity in a subject of acute pain comprising administering an effective amount of a compound of the invention, a pharmaceutically acceptable salt thereof or a pharmaceutical composition thereof. In some aspects, the acute pain comprises acute post-operative pain.

In yet another aspect, the invention features a method of treating or lessening the severity in a subject of postsurgical pain (e.g., joint replacement pain, soft tissue surgery pain, post-thoracotomy pain, post-mastectomy pain, hemorrhoidectomy pain, herniorrhaphy pain, bunionectomy pain or abdominoplasty pain) comprising administering an effective amount of a compound of the invention, a pharmaceutically acceptable salt thereof or a pharmaceutical composition thereof.

In yet another aspect, the invention features a method of treating or lessening the severity in a subject of bunionectomy pain comprising administering an effective amount of a compound of the invention, a pharmaceutically acceptable salt thereof or a pharmaceutical composition thereof.

In yet another aspect, the invention features a method of treating or lessening the severity in a subject of shoulder arthroplasty pain or shoulder arthroscopy pain comprising administering an effective amount of a compound of the invention, a pharmaceutically acceptable salt thereof or a pharmaceutical composition thereof.

In yet another aspect, the invention features a method of treating or lessening the severity in a subject of herniorrhaphy pain comprising administering an effective amount of a compound of the invention, a pharmaceutically acceptable salt thereof or a pharmaceutical composition thereof.

In yet another aspect, the invention features a method of treating or lessening the severity in a subject of abdominoplasty pain comprising administering an effective amount of a compound of the invention, a pharmaceutically acceptable salt thereof or a pharmaceutical composition thereof.

In yet another aspect, the invention features a method of treating or lessening the severity in a subject of visceral pain comprising administering an effective amount of a compound of the invention, a pharmaceutically acceptable salt thereof or a pharmaceutical composition thereof. In some aspects, the visceral pain comprises visceral pain from abdominoplasty.

In yet another aspect, the invention features a method of treating or lessening the severity in a subject of a neurodegenerative disease comprising administering an effective amount of a compound of the invention, a pharmaceutically acceptable salt thereof or a pharmaceutical composition thereof. In some aspects, the neurodegenerative disease comprises multiple sclerosis. In some aspects, the neurodegenerative disease comprises Pitt Hopkins Syndrome (PTHS).

In yet another aspect, the invention features a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the invention features a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Na_{V}1.8.

In another aspect, the invention features a method of treating or lessening the severity in a subject of acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal neuralgia, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility, comprising administering an effective amount of a compound of the invention, a pharmaceutically acceptable salt thereof or a pharmaceutical composition thereof.

In another aspect, the invention features a method of treating or lessening the severity in a subject of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain, comprising administering an effective amount of a compound of the invention, a pharmaceutically acceptable salt thereof or a pharmaceutical composition thereof.

In another aspect, the invention features a method of treating or lessening the severity in a subject of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbrosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain, comprising administering an effective amount of a compound of the invention, a pharmaceutically acceptable salt thereof or a pharmaceutical composition thereof.

### Compounds, Pharmaceutically Acceptable Salts, and Compositions for Use

In another aspect, the invention features a compound of the invention, or a pharmaceutically acceptable salt or pharmaceutical composition thereof, for use as a medicament.

In another aspect, the invention features a compound of the invention, or a pharmaceutically acceptable salt or pharmaceutical composition thereof, for use in a method of inhibiting a voltage-gated sodium channel in a subject. In another aspect, the voltage-gated sodium channel is Na_{V}1.8.

In another aspect, the invention features a compound of the invention, or a pharmaceutically acceptable salt or pharmaceutical composition thereof, for use in a method of treating or lessening the severity in a subject of chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia.

In another aspect, the invention features a compound of the invention, or a pharmaceutically acceptable salt or pharmaceutical composition thereof, for use in a method of treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia.

In another aspect, the invention features a compound of the invention, or a pharmaceutically acceptable salt or pharmaceutical composition thereof, for use in a method of treating or lessening the severity in a subject of gut pain, wherein gut pain comprises inflammatory bowel disease pain, Crohn's disease pain, irritable bowel syndrome, endometriosis, polycyctic ovarian disease, salpingitis, cervicitis or interstitial cystitis pain.

In another aspect, the invention features a compound of the invention, or a pharmaceutically acceptable salt or pharmaceutical composition thereof, for use in a method of treating or lessening the severity in a subject of neuropathic pain. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In another aspect, the invention features a compound of the invention, or a pharmaceutically acceptable salt or pharmaceutical composition thereof, for use in a method of treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic cephalalgia.

In another aspect, the invention features a compound of the invention, or a pharmaceutically acceptable salt or pharmaceutical composition thereof, for use in a method of treating or lessening the severity in a subject of musculoskeletal pain. In some aspects, the musculoskeletal pain comprises osteoarthritis pain.

In another aspect, the invention features a compound of the invention, or a pharmaceutically acceptable salt or pharmaceutical composition thereof, for use in a method of treating or lessening the severity in a subject of musculoskeletal pain, wherein musculoskeletal pain comprises osteoarthritis pain, back pain, cold pain, burn pain or dental pain.

In another aspect, the invention features a compound of the invention, or a pharmaceutically acceptable salt or pharmaceutical composition thereof, for use in a method of treating or lessening the severity in a subject of inflammatory pain, wherein inflammatory pain comprises rheumatoid arthritis pain, ankylosing spondylitis or vulvodynia.

In another aspect, the invention features a compound of the invention, or a pharmaceutically acceptable salt or pharmaceutical composition thereof, for use in a method of treating or lessening the severity in a subject of inflammatory pain, wherein inflammatory pain comprises rheumatoid arthritis pain.

In another aspect, the invention features a compound of the invention, or a pharmaceutically acceptable salt or pharmaceutical composition thereof, for use in a method of treating or lessening the severity in a subject of idiopathic pain, wherein idiopathic pain comprises fibromyalgia pain.

In yet another aspect, the invention features compound of the invention, or a pharmaceutically acceptable salt or pharmaceutical composition thereof, for use in a method of treating or lessening the severity in a subject of idiopathic pain, wherein idiopathic pain comprises reflex sympathetic dystrophy pain.

In another aspect, the invention features a compound of the invention, or a pharmaceutically acceptable salt or pharmaceutical composition thereof, for use in a method of treating or lessening the severity in a subject of pathological cough.

In another aspect, the invention features a compound of the invention, or a pharmaceutically acceptable salt or pharmaceutical composition thereof, for use in a method of treating or lessening the severity in a subject of acute pain. In some aspects, the acute pain comprises acute post-operative pain.

In another aspect, the invention features a compound of the invention, or a pharmaceutically acceptable salt or pharmaceutical composition thereof, for use in a method of treating or lessening the severity in a subject of postsurgical pain (e.g., joint replacement pain, soft tissue surgery pain, post-thoracotomy pain, post-mastectomy pain, hemorrhoidectomy pain, herniorrhaphy pain, bunionectomy pain or abdominoplasty pain).

In another aspect, the invention features a compound of the invention, or a pharmaceutically acceptable salt or pharmaceutical composition thereof, for use in a method of treating or lessening the severity in a subject of bunionectomy pain.

In yet another aspect, the invention features a compound of the invention, or a pharmaceutically acceptable salt or pharmaceutical composition thereof, for use in a method of treating or lessening the severity in a subject of shoulder arthroplasty pain or shoulder arthroscopy pain.

In another aspect, the invention features a compound of the invention, or a pharmaceutically acceptable salt or pharmaceutical composition thereof, for use in a method of treating or lessening the severity in a subject of herniorrhaphy pain.

In another aspect, the invention features a compound of the invention, or a pharmaceutically acceptable salt or pharmaceutical composition thereof, for use in a method of treating or lessening the severity in a subject of abdominoplasty pain.

In another aspect, the invention features a compound of the invention, or a pharmaceutically acceptable salt or pharmaceutical composition thereof, for use in a method of treating or lessening the severity in a subject of visceral pain. In some aspects, the visceral pain comprises visceral pain from abdominoplasty.

In another aspect, the invention features a compound of the invention, or a pharmaceutically acceptable salt or pharmaceutical composition thereof, for use in a method of treating or lessening the severity in a subject of a neurodegenerative disease. In some aspects, the neurodegenerative disease comprises multiple sclerosis. In some aspects, the neurodegenerative disease comprises Pitt Hopkins Syndrome (PTHS).

In another aspect, the invention features a compound of the invention, or a pharmaceutically acceptable salt or pharmaceutical composition thereof, for use in a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the invention features a compound of the invention, or a pharmaceutically acceptable salt or pharmaceutical composition thereof, for use in a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Na_{V}1.8.

In another aspect, the invention features a compound of the invention, or a pharmaceutically acceptable salt or pharmaceutical composition thereof, for use in a method of treating or lessening the severity in a subject of acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility.

In another aspect, the invention features a compound of the invention, or a pharmaceutically acceptable salt or pharmaceutical composition thereof, for use in a method of treating or lessening the severity in a subject of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain.

In another aspect, the invention features a compound of the invention, or a pharmaceutically acceptable salt or pharmaceutical composition thereof, for use in a method of treating or lessening the severity in a subject of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbrosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain.

### Manufacture of Medicaments

In another aspect, the invention provides the use of a compound of the invention, or a pharmaceutically acceptable salt or pharmaceutical composition thereof, for the manufacture of a medicament.

In another aspect, the invention provides the use of a compound of the invention, a pharmaceutically acceptable salt thereof, or a pharmaceutical composition thereof for the manufacture of a medicament for use in inhibiting a voltage-gated sodium channel. In another aspect, the voltage-gated sodium channel is Na_{V}1.8.

In yet another aspect, the invention provides the use of a compound of the invention, a pharmaceutically acceptable salt thereof, or a pharmaceutical composition thereof for the manufacture of a medicament for use in treating or lessening the severity in a subject of chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia.

In yet another aspect, the invention provides the use of a compound of the invention, a pharmaceutically acceptable salt thereof, or a pharmaceutical composition thereof for the manufacture of a medicament for use in treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia.

In yet another aspect, the invention provides the use of the compound, pharmaceutically acceptable salt, or pharmaceutical composition described herein for the manufacture of a medicament for use in treating or lessening the severity in a subject of gut pain, wherein gut pain comprises inflammatory bowel disease pain, Crohn's disease pain, irritable bowel syndrome, endometriosis, polycyctic ovarian disease, salpingitis, cervicitis or interstitial cystitis pain.

In yet another aspect, the invention provides a compound of the invention, a pharmaceutically acceptable salt thereof, or a pharmaceutical composition thereof for the manufacture of a medicament for use in treating or lessening the severity in a subject of neuropathic pain. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy).

In yet another aspect, the invention provides the use of a compound of the invention, a pharmaceutically acceptable salt thereof, or a pharmaceutical composition thereof for the manufacture of a medicament for use in a treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic neuropathy.

In yet another aspect, the invention provides the use of a compound of the invention, a pharmaceutically acceptable salt thereof, or a pharmaceutical composition thereof for the manufacture of a medicament for use in treating or lessening the severity in a subject of musculoskeletal pain. In some aspects the musculoskeletal pain comprises osteoarthritis pain.

In yet another aspect, the invention provides the use of a compound of the invention, a pharmaceutically acceptable salt thereof, or a pharmaceutical composition thereof for the manufacture of a medicament for use in treating or lessening the severity in a subject of musculoskeletal pain, wherein musculoskeletal pain comprises osteoarthritis pain, back pain, cold pain, burn pain or dental pain.

In yet another aspect, the invention provides the use of a compound of the invention, a pharmaceutically acceptable salt thereof, or a pharmaceutical composition thereof for the manufacture of a medicament for use in treating or lessening the severity in a subject of inflammatory pain, wherein inflammatory pain comprises rheumatoid arthritis pain, ankylosing spondylitis or vulvodynia.

In yet another aspect, the invention provides the use of a compound of the invention, a pharmaceutically acceptable salt thereof, or a pharmaceutical composition thereof for the manufacture of a medicament for use in treating or lessening the severity in a subject of inflammatory pain, wherein inflammatory pain comprises rheumatoid arthritis pain.

In yet another aspect, the invention provides the use of a compound of the invention, a pharmaceutically acceptable salt thereof, or a pharmaceutical composition thereof for the manufacture of a medicament for use in treating or lessening the severity in a subject of idiopathic pain, wherein idiopathic pain comprises fibromyalgia pain.

In yet another aspect, the invention provides for the use of a compound of the invention, a pharmaceutically acceptable salt thereof, or a pharmaceutical composition thereof for the manufacture of a medicament for use in treating or lessening the severity in a subject of idiopathic pain, wherein idiopathic pain comprises reflex sympathetic dystrophy pain.

In yet another aspect, the invention provides the use of a compound of the invention, a pharmaceutically acceptable salt thereof, or a pharmaceutical composition thereof for the manufacture of a medicament for use in treating or lessening the severity in a subject of pathological cough.

In yet another aspect, the invention provides the use of a compound of the invention, a pharmaceutically acceptable salt thereof, or a pharmaceutical composition thereof for the manufacture of a medicament for use in treating or lessening the severity in a subject of acute pain. In some aspects, the acute pain comprises acute post-operative pain.

In yet another aspect, the invention provides the use of a compound of the invention, a pharmaceutically acceptable salt thereof, or a pharmaceutical composition thereof for the manufacture of a medicament for use in treating or lessening the severity in a subject of postsurgical pain (e.g., joint replacement pain, soft tissue surgery pain, post-thoracotomy pain, post-mastectomy pain, hemorrhoidectomy pain, herniorrhaphy pain, bunionectomy pain or abdominoplasty pain).

In yet another aspect, the invention provides the use of a compound of the invention, a pharmaceutically acceptable salt thereof, or a pharmaceutical composition thereof for the manufacture of a medicament for use in treating or lessening the severity in a subject of herniorrhaphy pain.

In yet another aspect, the invention provides the use of a compound of the invention, a pharmaceutically acceptable salt thereof, or a pharmaceutical composition thereof for the manufacture of a medicament for use in treating or lessening the severity in a subject of bunionectomy pain.

In yet another aspect, the invention provides the use of a compound of the invention, a pharmaceutically acceptable salt thereof, or a pharmaceutical composition thereof for the manufacture of a medicament for use in treating or lessening the severity in a subject of shoulder arthroplasty pain or shoulder arthroscopy pain.

In yet another aspect, the invention provides the use of a compound of the invention, a pharmaceutically acceptable salt thereof, or a pharmaceutical composition thereof for the manufacture of a medicament for use in treating or lessening the severity in a subject of abdominoplasty pain.

In yet another aspect, the invention provides the use of a compound of the invention, a pharmaceutically acceptable salt thereof, or a pharmaceutical composition thereof for the manufacture of a medicament for use in treating or lessening the severity in a subject of visceral pain. In some aspects, the visceral pain comprises visceral pain from abdominoplasty.

In another aspect, the invention features a compound of the invention, or a pharmaceutically acceptable salt or pharmaceutical composition thereof, for the manufacture of a medicament for use in treating or lessening the severity in a subject of a neurodegenerative disease. In some aspects, the neurodegenerative disease comprises multiple sclerosis. In some aspects, the neurodegenerative disease comprises Pitt Hopkins Syndrome (PTHS).

In yet another aspect, the invention provides the use of a compound of the invention, a pharmaceutically acceptable salt thereof, or a pharmaceutical composition thereof for the manufacture of a medicament for use in combination with one or more additional therapeutic agents administered concurrently with, prior to, or subsequent to treatment with the compound or pharmaceutical composition. In some embodiments, the additional therapeutic agent is a sodium channel inhibitor.

In another aspect, the invention provides the use of a compound of the invention, a pharmaceutically acceptable salt thereof, or a pharmaceutical composition thereof for the manufacture of a medicament for use in treating or lessening the severity of acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia reumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodyscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarhropathy, temporo-mandibular joint disorder, painful joint arthroplasties, non-cardiac chest pain, pudendal, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility.

In another aspect, the invention provides the use of a compound of the invention, a pharmaceutically acceptable salt thereof, or a pharmaceutical composition thereof for the manufacture of a medicament for use in treating or lessening the severity of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behect's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain.

In another aspect, the invention provides the use of a compound of the invention, a pharmaceutically acceptable salt thereof, or a pharmaceutical composition thereof for the manufacture of a medicament for use in treating or lessening the severity of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbrosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain.

### Administration of Compounds, Pharmaceutically Acceptable Salts, and Compositions

In certain embodiments of the invention an “effective amount” of a compound of the invention, a pharmaceutically acceptable salt thereof, or a pharmaceutical composition thereof is that amount effective for treating or lessening the severity of one or more of the conditions recited above.

The compounds, salts, and compositions, according to the method of the invention, may be administered using any amount and any route of administration effective for treating or lessening the severity of one or more of the pain or non-pain diseases recited herein. The exact amount required will vary from subject to subject, depending on the species, age, and general condition of the subject, the severity of the condition, the particular agent, its mode of administration, and the like. The compounds, salts, and compositions of the invention are preferably formulated in dosage unit form for ease of administration and uniformity of dosage. The expression “dosage unit form” as used herein refers to a physically discrete unit of agent appropriate for the subject to be treated. It will be understood, however, that the total daily usage of the compounds, salts, and compositions of the invention will be decided by the attending physician within the scope of sound medical judgment. The specific effective dose level for any particular subject or organism will depend upon a variety of factors including the disorder being treated and the severity of the disorder; the activity of the specific compound or salt employed; the specific composition employed; the age, body weight, general health, sex and diet of the subject; the time of administration, route of administration, and rate of excretion of the specific compound or salt employed; the duration of the treatment; drugs used in combination or coincidental with the specific compound or salt employed, and like factors well known in the medical arts. The term “subject” or “patient,” as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the condition being treated. In certain embodiments, the compound, salts, and compositions of the invention may be administered orally or parenterally at dosage levels of about 0.001 mg/kg to about 1000 mg/kg, one or more times a day, effective to obtain the desired therapeutic effect.

Liquid dosage forms for oral administration include, but are not limited to, pharmaceutically acceptable emulsions, microemulsions, solutions, suspensions, syrups and elixirs. In addition to the active compound or salt, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of the compounds of the invention, it is often desirable to slow the absorption of the compounds from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compound or salt of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound or salt is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compound or salt can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound or salt may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound or salt of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As described generally above, the compounds of the invention are useful as inhibitors of voltage-gated sodium channels. In one embodiment, the compounds are inhibitors of Na_{V}1.8 and thus, without wishing to be bound by any particular theory, the compounds, salts, and compositions are particularly useful for treating or lessening the severity of a disease, condition, or disorder where activation or hyperactivity of Na_{V}1.8 is implicated in the disease, condition, or disorder. When activation or hyperactivity of Na_{V}1.8 is implicated in a particular disease, condition, or disorder, the disease, condition, or disorder may also be referred to as a “Na_{V}1.8-mediated disease, condition or disorder.” Accordingly, in another aspect, the invention provides a method for treating or lessening the severity of a disease, condition, or disorder where activation or hyperactivity of Na_{V}1.8 is implicated in the disease state.

The activity of a compound utilized in this invention as an inhibitor of Na_{V}1.8 may be assayed according to methods described generally in International Publication No. WO 2014/120808 A9 and U.S. Publication No. 2014/0213616 A1, both of which are incorporated by reference in their entirety, methods described herein, and other methods known and available to one of ordinary skill in the art.

### Additional Therapeutic Agents

It will also be appreciated that the compounds, salts, and pharmaceutically acceptable compositions of the invention can be employed in combination therapies, that is, the compounds, salts, and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated.” For example, exemplary additional therapeutic agents include, but are not limited to: non-opioid analgesics (indoles such as Etodolac, Indomethacin, Sulindac, Tolmetin; naphthylalkanones such as Nabumetone; oxicams such as Piroxicam; para-aminophenol derivatives, such as Acetaminophen; propionic acids such as Fenoprofen, Flurbiprofen, Ibuprofen, Ketoprofen, Naproxen, Naproxen sodium, Oxaprozin; salicylates such as Aspirin, Choline magnesium trisalicylate, Diflunisal; fenamates such as meclofenamic acid, Mefenamic acid; and pyrazoles such as Phenylbutazone); or opioid (narcotic) agonists (such as Codeine, Fentanyl, Hydromorphone, Levorphanol, Meperidine, Methadone, Morphine, Oxycodone, Oxymorphone, Propoxyphene, Buprenorphine, Butorphanol, Dezocine, Nalbuphine, and Pentazocine). Additionally, nondrug analgesic approaches may be utilized in conjunction with administration of one or more compounds of the invention. For example, anesthesiologic (intraspinal infusion, neural blockade), neurosurgical (neurolysis of CNS pathways), neurostimulatory (transcutaneous electrical nerve stimulation, dorsal column stimulation), physiatric (physical therapy, orthotic devices, diathermy), or psychologic (cognitive methods-hypnosis, biofeedback, or behavioral methods) approaches may also be utilized. Additional appropriate therapeutic agents or approaches are described generally in The Merck Manual, Nineteenth Edition, Ed. Robert S. Porter and Justin L. Kaplan, Merck Sharp & Dohme Corp., a subsidiary of Merck & Co., Inc., 2011, and the Food and Drug Administration website, www.fda.gov, the entire contents of which are hereby incorporated by reference.

In another embodiment, additional appropriate therapeutic agents are selected from the following:
(1) an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine, pentazocine, or difelikefalin;
(2) a nonsteroidal antiinflammatory drug (NSAID), e.g. aspirin, diclofenac, diflunisal, etodolac, fenbufen, fenoprofen, flufenisal, flurbiprofen, ibuprofen (including without limitation intravenous ibuprofen (e.g., Caldolor®)), indomethacin, ketoprofen, ketorolac (including without limitation ketorolac tromethamine (e.g., Toradol®)), meclofenamic acid, mefenamic acid, meloxicam, IV meloxicam (e.g., Anjeso®), nabumetone, naproxen, nimesulide, nitroflurbiprofen, olsalazine, oxaprozin, phenylbutazone, piroxicam, sulfasalazine, sulindac, tolmetin or zomepirac;
(3) a barbiturate sedative, e.g. amobarbital, aprobarbital, butabarbital, butalbital, mephobarbital, metharbital, methohexital, pentobarbital, phenobarbital, secobarbital, talbutal, thiamylal or thiopental;
(4) a benzodiazepine having a sedative action, e.g. chlordiazepoxide, clorazepate, diazepam, flurazepam, lorazepam, oxazepam, temazepam or triazolam;
(5) a histamine (H₁) antagonist having a sedative action, e.g. diphenhydramine, pyrilamine, promethazine, chlorpheniramine or chlorcyclizine;
(6) a sedative such as glutethimide, meprobamate, methaqualone or dichloralphenazone;
(7) a skeletal muscle relaxant, e.g. baclofen, carisoprodol, chlorzoxazone, cyclobenzaprine, methocarbamol or orphenadrine;
(8) an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®), a combination formulation of morphine and dextromethorphan), topiramate, neramexane or perzinfotel including an NR2B antagonist, e.g. ifenprodil, traxoprodil or (−)(R)-6-{2-[4-(3-fluorophenyl)-4-hydroxy-1-piperidinyl]-1-hydroxyethyl-3,4-dihydro-2(1H)-quinolinone;
(9) an alpha-adrenergic, e.g. doxazosin, tamsulosin, clonidine, guanfacine, dexmedetomidine, modafinil, or 4-amino-6,7-dimethoxy-2-(5-methane-sulfonamido-1,2,3,4-tetrahydroisoquinolin-2-yl)-5-(2-pyridyl) quinazoline;
(10) a tricyclic antidepressant, e.g. desipramine, imipramine, amitriptyline or nortriptyline;
(11) an anticonvulsant, e.g. carbamazepine (Tegretol®), lamotrigine, topiramate, lacosamide (Vimpat®) or valproate;
(12) a tachykinin (NK) antagonist, particularly an NK-3, NK-2 or NK-1 antagonist, e.g. (alphaR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
(13) a muscarinic antagonist, e.g oxybutynin, tolterodine, propiverine, tropsium chloride, darifenacin, solifenacin, temiverine and ipratropium;
(14) a COX-2 selective inhibitor, e.g. celecoxib, rofecoxib, parecoxib, valdecoxib, deracoxib, etoricoxib, or lumiracoxib;
(15) a coal-tar analgesic, in particular paracetamol;
(16) a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, belaperidone, palindore, cplivanscrin, osanctant, rimonabant, meclinertant, Miraxion® or sarizotan;
(17) a vanilloid receptor agonist (e.g. resinferatoxin or civamide) or antagonist (e.g. capsazepine, GRC-15300);
(18) a beta-adrenergic such as propranolol;
(19) a local anesthetic such as mexiletine;
(20) a corticosteroid such as dexamethasone;
(21) a 5-HT receptor agonist or antagonist, particularly a 5-HT_{1B/1D} agonist such as eletriptan, sumatriptan, naratriptan, zolmitriptan or rizatriptan;
(22) a 5-HT₂A receptor antagonist such as R(+)-alpha-(2,3-dimethoxy-phenyl)-1-[2-(4-fluorophenylethyl)]-4-piperidinemethanol (MDL-100907);
(23) a cholinergic (nicotinic) analgesic, such as ispronieline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
(24) Tramadol®, Tramadol ER (Ultram ER®), IV Tramadol, Tapentadol ER (Nucynta®);
(25) a PDE5 inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
(26) an alpha-2-delta ligand such as gabapentin (Neurontin®), gabapentin GR (Gralise®), gabapentin, enacarbil (Horizant®), pregabalin (Lyrica®), 3-methyl gabapentin, (1[alpha],3[alpha],5[alpha])(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
(27) a cannabinoid such as KHK-6188;
(28) metabotropic glutamate subtype 1 receptor (mGluR1) antagonist;
(29) a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
(30) a noradrenaline (norepinephrine) reuptake inhibitor, such as maprotiline, lofepramine, mirtazepine, oxaprotiline, fezolamine, tomoxetine, mianserin, bupropion, bupropion metabolite hydroxybupropion, nomifensine and viloxazine (Vivalan®), especially a selective noradrenaline reuptake inhibitor such as reboxetine, in particular (S,S)-reboxetine;
(31) a dual serotonin-noradrenaline reuptake inhibitor, such as venlafaxine, venlafaxine metabolite O-desmethylvenlafaxine, clomipramine, clomipramine metabolite desmethylclomipramine, duloxetine (Cymbalta®), milnacipran and imipramine;
(32) an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-S-chloro-S-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl)butyl]thio]-6-(trifluoromethyl)-3-pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, NXN-462, or guanidinoethyldisulfide;
(33) an acetylcholinesterase inhibitor such as donepezil;
(34) a prostaglandin E2 subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(15)-1-({[5-chloro-2-(3-fluorophenoxy)pyridin-3-yl]carbonyl}amino)ethyl]benzoic acid;
(35) a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870;
(36) a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl)-1,4-benzoquinone (CV-6504);
(37) a sodium channel blocker, such as lidocaine, lidocaine plus tetracaine cream (ZRS-201) or eslicarbazepine acetate;
(38) a Na_{V}1.7 blocker, such as XEN-402, XEN403, TV-45070, PF-05089771, CNV1014802, GDC-0276, RG7893 BIIB-074 (Vixotrigine), BIIB-095, ASP-1807, DSP-3905, OLP-1002, RQ-00432979, FX-301, DWP-1706, DWP-17061, IMB-110, IMB-111, IMB-112 and such as those disclosed in WO2011/140425 (US2011/306607); WO2012/106499 (US2012196869); WO2012/112743 (US2012245136); WO2012/125613 (US2012264749), WO2012/116440 (US2014187533), WO2011026240 (US2012220605), U.S. Pat. Nos. 8,883,840, 8,466,188, WO2013/109521 (US2015005304), CN111217776, WO2020/117626, WO2021/252822, WO2021/252818, WO2021/252820, WO2014/201173, WO2012/125973, WO2013/086229, WO2013/134518, WO2014/201206, or WO2016/141035 the entire contents of each application hereby incorporated by reference;
(38a) a Na_{V}1.7 blocker such as (2-benzylspiro[3,4-dihydropyrrolo[1,2-a]pyrazine-1,4′-piperidine]-1′-yl)-(4-isopropoxy-3-methyl-phenyl)methanone, 2,2,2-trifluoro-1-[1′-[3-methoxy-4-[2-(trifluoromethoxy)ethoxy]benzoyl]-2,4-dimethyl-spiro[3,4-dihydropyrrolo[1,2-a]pyrazine-1,4′-piperidine]-6-yl]ethanone, [8-fluoro-2-methyl-6-(trifluoromethyl)spiro[3,4-dihydropyrrolo[1,2-a]pyrazine-1,4′-piperidine]-1′-yl]-(4-isobutoxy-3-methoxy-phenyl)methanone, 1-(4-benzhydrylpiperazin-1-yl)-3-[2-(3,4-dimethylphenoxy)ethoxy]propan-2-ol, (4-butoxy-3-methoxy-phenyl)-[2-methyl-6-(trifluoromethyl)spiro[3,4-dihydropyrrolo[1,2-a]pyrazine-1,4′-piperidine]-1′-yl]methanone, [8-fluoro-2-methyl-6-(trifluoromethyl)spiro[3,4-dihydropyrrolo[1,2-a]pyrazine-1,4′-piperidine]-1′-yl]-(5-isopropoxy-6-methyl-2-pyridyl)methanone, (4-isopropoxy-3-methyl-phenyl)-[2-methyl-6-(1,1,2,2,2-pentafluoroethyl)spiro[3,4-dihydropyrrolo[1,2-a]pyrazine-1,4′-piperidine]-1′-yl]methanone, 5-[2-methyl-4-[2-methyl-6-(2,2,2-trifluoroacetyl)spiro[3,4-dihydropyrrolo[1,2-a]pyrazine-1,4′-piperidine]-1′-carbonyl]phenyl]pyridine-2-carbonitrile, (4-isopropoxy-3-methyl-phenyl)-[6-(trifluoromethyl)spiro[3,4-dihydro-2H-pyrrolo[1,2-a]pyrazine-1,4′-piperidine]-1′-yl]methanone, 2,2,2-trifluoro-1-[1′-[3-methoxy-4-[2-(trifluoromethoxy)ethoxy]benzoyl]-2-methyl-spiro[3,4-dihydropyrrolo[1,2-a]pyrazine-1,4′-piperidine]-6-yl]ethanone, 2,2,2-trifluoro-1-[1′-(5-isopropoxy-6-methyl-pyridine-2-carbonyl)-3,3-dimethyl-spiro[2,4-dihydropyrrolo[1,2-a]pyrazine-1,4′-piperidine]-6-yl]ethanone, 2,2,2-trifluoro-1-[1′-(5-isopentyloxypyridine-2-carbonyl)-2-methyl-spiro[3,4-dihydropyrrolo[1,2-a]pyrazine-1,4′-piperidine]-6-yl]ethanone, (4-isopropoxy-3-methoxy-phenyl)-[2-methyl-6-(trifluoromethyl)spiro[3,4-dihydropyrrolo[1,2-a]pyrazine-1,4′-piperidine]-1′-yl]methanone, 2,2,2-trifluoro-1-[1′-(5-isopentyloxypyridine-2-carbonyl)-2,4-dimethyl-spiro[3,4-dihydropyrrolo[1,2-a]pyrazine-1,4′-piperidine]-6-yl]ethanone, 1-[(3S)-2,3-dimethyl-1′-[4-(3,3,3-trifluoropropoxymethyl)benzoyl]spiro[3,4-dihydropyrrolo[1,2-a]pyrazine-1,4′-piperidine]-6-yl]-2,2,2-trifluoro-ethanone, [8-fluoro-2-methyl-6-(trifluoromethyl)spiro[3,4-dihydropyrrolo[1,2-a]pyrazine-1,4′-piperidine]-1′-yl]-[3-methoxy-4-[(1R)-1-methylpropoxy]phenyl]methanone, 2,2,2-trifluoro-1-[1′-(5-isopropoxy-6-methyl-pyridine-2-carbonyl)-2,4-dimethyl-spiro[3,4-dihydropyrrolo[1,2-a]pyrazine-1,4′-piperidine]-6-yl]ethanone, 1-[1′-[4-methoxy-3-(trifluoromethyl)benzoyl]-2-methyl-spiro[3,4-dihydropyrrolo[1,2-a]pyrazine-1,4′-piperidine]-6-yl]-2,2-dimethyl-propan-1-one, (4-isopropoxy-3-methyl-phenyl)-[2-methyl-6-(trifluoromethyl)spiro[3,4-dihydropyrrolo[1,2-a]pyrazine-1,4′-piperidine]-1′-yl]methanone, [2-methyl-6-(1-methylcyclopropanecarbonyl)spiro[3,4-dihydropyrrolo[1,2-a]pyrazine-1,4′-piperidine]-1′-yl]-[4-(3,3,3-trifluoropropoxymethyl)phenyl]methanone, 4-bromo-N-(4-bromophenyl)-3-[(1-methyl-2-oxo-4-piperidyl)sulfamoyl]benzamide or (3-chloro-4-isopropoxy-phenyl)-[2-methyl-6-(1,1,2,2,2-pentafluoroethyl)spiro[3,4-dihydropyrrolo[1,2-a]pyrazine-1,4′-piperidine]-1′-yl]methanone.

(39) a Na_{V}1.8 blocker, such as PF-04531083, PF-06372865 and such as those disclosed in WO2008/135826 (US2009048306), WO2006/011050 (US2008312235), WO2013/061205 (US2014296313), US20130303535, WO2013131018, U.S. Pat. No. 8,466,188, WO2013114250 (US2013274243), WO2014/120808 (US2014213616), WO2014/120815 (US2014228371) WO2014/120820 (US2014221435), WO2015/010065 (US20160152561), WO2015/089361 (US20150166589), WO2019/014352 (US20190016671), WO2018/213426, WO2020/146682, WO2020/146612, WO2020/014243, WO2020/014246, WO2020/092187, WO2020/092667 (US2020140411), WO2020/144375, WO2020/261114, WO2020/140959, WO2020/151728, WO2021/032074, WO2021/047622 (CN112479996), WO2021/257490, WO/2021/257420, WO2021/257418, WO2022/263498, WO2022/235558, WO2022/235859, CN112390745, CN111808019, CN112225695, CN112457294, CN112300051, CN112300069, CN112441969, and CN114591293, the entire contents of each application hereby incorporated by reference;
(39a) a Na_{V}1.8 blocker such as 4,5-dichloro-2-(4-fluoro-2-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-4-(perfluoroethyl)benzamide, 4,5-dichloro-2-(4-fluorophenoxy)-N-(2-oxo-1,2-dihydropyridin-4-yl)benzamide, 4,5-dichloro-2-(3-fluoro-4-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-5-(trifluoromethyl)benzamide, N-(2-oxo-1,2-dihydropyridin-4-yl)-2-(4-(trifluoromethoxy)phenoxy)-4-(trifluoromethyl)benzamide, 2-(4-fluorophenoxy)-N-(2-oxo-1,2-dihydropyridin-4-yl)-4-(perfluoroethyl)benzamide, 5-chloro-2-(4-fluoro-2-methoxyphenoxy)-N-(2-oxo-1,2-dihydropyridin-4-yl)benzamide, N-(2-oxo-1,2-dihydropyridin-4-yl)-2-(4-(trifluoromethoxy)phenoxy)-5-(trifluoromethyl)benzamide, 2-(4-fluoro-2-methylphenoxy)-N-(2-oxo-1,2-dihydropyridin-4-yl)-5-(trifluoromethyl)benzamide, 2-(2-chloro-4-fluorophenoxy)-N-(2-oxo-1,2-dihydropyridin-4-yl)-5-(trifluoromethyl)benzamide, 5-chloro-2-(4-fluoro-2-methylphenoxy)-N-(2-oxo-1,2-dihydropyridin-4-yl)benzamide, 4-chloro-2-(4-fluoro-2-methylphenoxy)-N-(2-oxo-1,2-dihydropyridin-4-yl)benzamide, 5-chloro-2-(2-chloro-4-fluorophenoxy)-N-(2-oxo-1,2-dihydropyridin-4-yl)benzamide, 2-((5-fluoro-2-hydroxybenzyl)oxy)-N-(2-oxo-1,2-dihydropyridin-4-yl)-4-(trifluoromethyl)benzamide, N-(2-oxo-1,2-dihydropyridin-4-yl)-2-(o-tolyloxy)-5-(trifluoromethyl)benzamide, 2-(2,4-difluorophenoxy)-N-(2-oxo-1,2-dihydropyridin-4-yl)-4-(trifluoromethyl)benzamide, N-(2-oxo-1,2-dihydropyridin-4-yl)-2-(2-(trifluoromethoxy)phenoxy)-5-(trifluoromethyl)benzamide, 2-(4-fluorophenoxy)-N-(2-oxo-1,2-dihydropyridin-4-yl)-5-(trifluoromethyl)benzamide, 2-(4-fluoro-2-methyl-phenoxy)-N-(2-oxo-1H-pyridin-4-yl)-4-(trifluoromethyl)benzamide, [4-[[2-(4-fluoro-2-methyl-phenoxy)-4-(trifluoromethyl)benzoyl]amino]-2-oxo-1-pyridyl]methyl dihydrogen phosphate, 2-(4-fluoro-2-(methyl-d₃)phenoxy)-N-(2-oxo-1,2-dihydropyridin-4-yl)-4-(trifluoromethyl)benzamide, (4-(2-(4-fluoro-2-(methyl-d₃)phenoxy)-4-(trifluoromethyl)benzamido)-2-oxopyridin-1(2H)-yl)methyl dihydrogen phosphate, 3-(4-fluoro-2-methoxyphenoxy)-N-(3-(methylsulfonyl)phenyl)quinoxaline-2-carboxamide, 3-(2-chloro-4-fluorophenoxy)-N-(3-sulfamoylphenyl)quinoxaline-2-carboxamide, 3-(2-chloro-4-methoxyphenoxy)-N-(3-sulfamoylphenyl)quinoxaline-2-carboxamide, 3-(4-chloro-2-methoxyphenoxy)-N-(3-sulfamoylphenyl)quinoxaline-2-carboxamide, 4-(3-(4-(trifluoromethoxy)phenoxy)quinoxaline-2-carboxamido)picolinic acid, 2-(2,4-difluorophenoxy)-N-(3-sulfamoylphenyl)quinoline-3-carboxamide, 2-(4-fluoro-2-methoxyphenoxy)-N-(3-sulfamoylphenyl)quinoline-3-carboxamide, 3-(2,4-difluorophenoxy)-N-(3-sulfamoylphenyl)quinoxaline-2-carboxamide, N-(3-sulfamoylphenyl)-2-(4-(trifluoromethoxy)phenoxy)quinoline-3-carboxamide, N-(3-sulfamoylphenyl)-3-(4-(trifluoromethoxy)phenoxy)quinoxaline-2-carboxamide, 3-(4-chloro-2-methylphenoxy)-N-(3-sulfamoylphenyl)quinoxaline-2-carboxamide, 5-(3-(4-(trifluoromethoxy)phenoxy)quinoxaline-2-carboxamido)picolinic acid, 3-(4-fluoro-2-methoxyphenoxy)-N-(2-oxo-2,3-dihydro-1H-benzo[d]imidazol-5-yl)quinoxaline-2-carboxamide, 3-(4-fluoro-2-methoxyphenoxy)-N-(pyridin-4-yl)quinoxaline-2-carboxamide, 3-(4-fluorophenoxy)-N-(3-sulfamoylphenyl)quinoxaline-2-carboxamide, N-(3-cyanophenyl)-3-(4-fluoro-2-methoxyphenoxy)quinoxaline-2-carboxamide, N-(4-carbamoylphenyl)-3-(4-fluoro-2-methoxyphenoxy)quinoxaline-2-carboxamide, 4-(3-(4-(trifluoromethoxy)phenoxy)quinoxaline-2-carboxamido)benzoic acid, N-(4-cyanophenyl)-3-(4-fluoro-2-methoxyphenoxy)quinoxaline-2-carboxamide, 5-(4,5-dichloro-2-(4-fluoro-2-methoxyphenoxy)benzamido)picolinic acid, 5-(2-(2,4-dimethoxyphenoxy)-4,6-bis(trifluoromethyl)benzamido)picolinic acid, 4-(4,5-dichloro-2-(4-fluoro-2-methoxyphenoxy)benzamido)benzoic acid, 5-(2-(4-fluoro-2-methoxyphenoxy)-4,6-bis(trifluoromethyl)benzamido)picolinic acid, 4-(2-(4-fluoro-2-methoxyphenoxy)-4-(perfluoroethyl)benzamido)benzoic acid, 5-(2-(4-fluoro-2-methoxyphenoxy)-4-(perfluoroethyl)benzamido)picolinic acid, 4-(2-(4-fluoro-2-methylphenoxy)-4-(trifluoromethyl)benzamido)benzoic acid, 5-(4,5-dichloro-2-(4-fluoro-2-methoxyphenoxy)benzamido)picolinic acid, 4-(2-(2-chloro-4-fluorophenoxy)-4-(perfluoroethyl)benzamido)benzoic acid, 4-(2-(4-fluoro-2-methylphenoxy)-4-(perfluoroethyl)benzamido)benzoic acid, 4-(4,5-dichloro-2-(4-(trifluoromethoxy)phenoxy)benzamido)benzoic acid, 4-(4,5-dichloro-2-(4-chloro-2-methylphenoxy)benzamido)benzoic acid, 5-(4-(tert-butyl)-2-(4-fluoro-2-methoxyphenoxy)benzamido)picolinic acid, 5-(4,5-dichloro-2-(4-(trifluoromethoxy)phenoxy)benzamido)picolinic acid, 4-(4,5-dichloro-2-(4-fluoro-2-methylphenoxy)benzamido)benzoic acid, 5-(4,5-dichloro-2-(2,4-dimethoxyphenoxy)benzamido)picolinic acid, 5-(4,5-dichloro-2-(2-chloro-4-fluorophenoxy)benzamido)picolinic acid, 5-(4,5-dichloro-2-(4-fluoro-2-methylphenoxy)benzamido)picolinic acid, 4-(4,5-dichloro-2-(4-chloro-2-methoxyphenoxy)benzamido)benzoic acid, 5-(4,5-dichloro-2-(2,4-difluorophenoxy)benzamido)picolinic acid, 2-(4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-6-(trifluoromethyl)benzamide, 2-(2-chloro-4-fluorophenoxy)-5-(difluoromethyl)-N-(3-sulfamoylphenyl)benzamide, 2-(4-fluorophenoxy)-4-(perfluoroethyl)-N-(3-sulfamoylphenyl)benzamide, 2-(4-chloro-2-methoxyphenoxy)-4-(perfluoroethyl)-N-(3-sulfamoylphenyl)benzamide, 2-(4-fluoro-2-methoxyphenoxy)-N-(3-sulfamoylphenyl)-5-(trifluoromethyl)benzamide, 5-chloro-2-(4-fluoro-2-methylphenoxy)-N-(3-sulfamoylphenyl)benzamide, 4,5-dichloro-2-(4-fluoro-2-methoxyphenoxy)-N-(3-sulfamoylphenyl)benzamide, 2,4-dichloro-6-(4-chloro-2-methoxyphenoxy)-N-(3-sulfamoylphenyl)benzamide, 2,4-dichloro-6-(4-fluoro-2-methylphenoxy)-N-(3-sulfamoylphenyl)benzamide, 2-(4-fluoro-2-methoxyphenoxy)-N-(3-sulfamoylphenyl)-4,6-bis(trifluoromethyl)benzamide, 2-(4-fluoro-2-methylphenoxy)-N-(3-sulfamoylphenyl)-4,6-bis(trifluoromethyl)benzamide, 5-chloro-2-(2-chloro-4-fluorophenoxy)-N-(3-sulfamoylphenyl)benzamide, 2-(4-fluoro-2-methoxyphenoxy)-N-(3-sulfamoylphenyl)-4-(trifluoromethoxy)benzamide, 2-(4-fluoro-2-methoxyphenoxy)-N-(3-sulfamoylphenyl)-4-(trifluoromethyl)benzamide, 4,5-dichloro-2-(4-fluorophenoxy)-N-(3-sulfamoylphenyl)benzamide, 2-(4-fluoro-2-methoxyphenoxy)-4-(perfluoroethyl)-N-(3-sulfamoylphenyl)benzamide, 5-fluoro-2-(4-fluoro-2-methylphenoxy)-N-(3-sulfamoylphenyl)benzamide, 2-(2-chloro-4-fluorophenoxy)-4-cyano-N-(3-sulfamoylphenyl)benzamide, N-(3-sulfamoylphenyl)-2-(4-(trifluoromethoxy)phenoxy)-4-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethyl)benzamide, N-(3-carbamoyl-4-fluoro-phenyl)-2-fluoro-6-[2-methoxy-4-(trifluoromethoxy)phenoxy]-3-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethoxy)benzamide, 4-[[2-fluoro-6-[2-methoxy-4-(trifluoromethoxy)phenoxyl-3-(trifluoromethyl)benzoyl]amino]pyridine-2-carboxamide, 4-[[3-chloro-2-fluoro-6-[2-methoxy-4-(trifluoromethoxy)phenoxy]benzoyl]amino]pyridine-2-carboxamide, 4-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-3-(difluoromethyl)-2-fluoro-6-[2-methoxy-4-(trifluoromethoxy)phenoxy]benzamide, 4-[[2-fluoro-6-[2-(trideuteriomethoxy)-4-(trifluoromethoxy)phenoxy]-3-(trifluoromethoxy)benzoyl]amino]pyridine-2-carboxamide, N-(3-carbamoyl-4-fluoro-phenyl)-6-[2-chloro-4-(trifluoromethoxy)phenoxy]-2-fluoro-3-(trifluoromethyl)benzamide, N-(3-carbamoyl-4-fluoro-phenyl)-2-fluoro-6-[2-methyl-4-(trifluoromethoxy)phenoxy]-3-(trifluoromethyl)benzamide, N-(3-carbamoyl-4-fluoro-phenyl)-2,3,4-trifluoro-6-[2-methoxy-4-(trifluoromethoxy)phenoxy]benzamide, N-(2-carbamoyl-4-pyridyl)-3-fluoro-5-[2-methoxy-4-(trifluoromethoxy)phenoxy]-2-(trifluoromethyl)pyridine-4-carboxamide, 4-[[6-[2-(difluoromethoxy)-4-(trifluoromethoxy)phenoxy]-2-fluoro-3-(trifluoromethyl)benzoyl]amino]pyridine-2-carboxamide, N-(3-carbamoyl-4-fluoro-phenyl)-6-[3-chloro-4-(trifluoromethoxy)phenoxy]-2-fluoro-3-(trifluoromethyl)benzamide, N-(3-carbamoyl-4-fluoro-phenyl)-2-fluoro-6-[4-(trifluoromethoxy)phenoxy]-3-(trifluoromethyl)benzamide, N-(4-carbamoyl-3-fluoro-phenyl)-2-fluoro-6-[2-methoxy-4-(trifluoromethoxy)phenoxy]-3-(trifluoromethyl)benzamide, 4-[[2-fluoro-6-[2-(trideuteriomethoxy)-4-(trifluoromethoxy)phenoxy]-4-(trifluoromethyl)benzoyl]amino]pyridine-2-carboxamide, N-(3-carbamoyl-4-fluoro-phenyl)-2-fluoro-6-[3-fluoro-4-(trifluoromethoxy)phenoxy]-3-(trifluoromethyl)benzamide, N-(3-carbamoyl-4-fluoro-phenyl)-2-[2-methoxy-4-(trifluoromethoxy)phenoxy]-5-(1,1,2,2,2-pentafluoroethyl)benzamide, 4-[[4-(difluoromethoxy)-2-fluoro-6-[2-methoxy-4-(trifluoromethoxy)phenoxy]benzoyl]amino]pyridine-2-carboxamide, N-(3-carbamoyl-4-fluoro-phenyl)-2-fluoro-6-[2-fluoro-4-(trifluoromethoxy)phenoxy]-3-(trifluoromethyl)benzamide, 4-[[4-cyclopropyl-2-fluoro-6-[2-methoxy-4-(trifluoromethoxy)phenoxy]benzoyl]amino]pyridine-2-carboxamide, N-(3-carbamoyl-4-fluoro-phenyl)-5-fluoro-2-[2-methoxy-4-(trifluoromethoxy)phenoxy]-4-(trifluoromethyl)benzamide, 5-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-2-fluoro-6-(4-fluorophenoxy)-3-(trifluoromethyl)benzamide, or 4-[[2-fluoro-6-[3-fluoro-2-methoxy-4-(trifluoromethoxy)phenoxy]-3-(trifluoromethyl)benzoyl]amino]pyridine-2-carboxamide;
(40) a combined Na_{V}1.7 and Na_{V}1.8 blocker, such as DSP-2230, Lohocla201 or BL-1021;
(41) a 5-HT3 antagonist, such as ondansetron;
(42) a TPRV 1 receptor agonist, such as capsaicin (NeurogesX®, Qutenza®); and the pharmaceutically acceptable salts and solvates thereof;
(43) a nicotinic receptor antagonist, such as varenicline;
(44) an N-type calcium channel antagonist, such as Z-160;
(45) a nerve growth factor antagonist, such as tanezumab;
(46) an endopeptidase stimulant, such as senrebotase;
(47) an angiotensin II antagonist, such as EMA-401;
(48) acetaminophen (including without limitation intravenous acetaminophen (e.g., Ofirmev®));
(49) bupivacaine (including without limitation bupivacaine liposome injectable suspension (e.g., Exparel®) bupivacaine ER (Posimir), bupivacaine collagen (Xaracoll) and transdermal bupivacaine (Eladur®)); and
(50) bupivacaine and meloxicam combination (e.g., HTX-011).

In one embodiment, the additional appropriate therapeutic agents are selected from V-116517, Pregabalin, controlled release Pregabalin, Ezogabine (Potiga®). Ketamine/amitriptyline topical cream (Amiket®), AVP-923, Perampanel (E-2007), Ralfnamide, transdermal bupivacaine (Eladur®), CNV1014802, JNJ-10234094 (Carisbamate), BMS-954561 or ARC-4558.

In another embodiment, the additional appropriate therapeutic agents are selected from N-(6-amino-5-(2,3,5-trichlorophenyl)pyridin-2-yl)acetamide; N-(6-amino-5-(2-chloro-5-methoxyphenyl)pyridin-2-yl)-1-methyl-1H-pyrazole-5-carboxamide; or 3-((4-(4-(trifluoromethoxy)phenyl)-1H-imidazol-2-yl)methyl)oxetan-3-amine.

In another embodiment, the additional therapeutic agent is selected from a GlyT2/5HT2 inhibitor, such as Operanserin (VVZ149), a TRPV modulator such as CA008, CMX-020, NE06860, FTABS, CNTX4975, MCP101, MDR16523, or MDR652, a EGR1 inhibitor such as Brivoglidc (AYX1), an NGF inhibitor such as Tanezumab, Fasinumab, ASP6294, MED17352, a Mu opioid agonist such as Cebranopadol, NKTR181 (oxycodegol), a CB-1 agonist such as NE01940 (AZN1940), an imidazoline 12 agonist such as CR4056 or a p75NTR-Fc modulator such as LEVI-04.

In another embodiment, the additional therapeutic agent is oliceridine or ropivacaine (TLC590).

In another embodiment, the additional therapeutic agent is a Na_{V}1.7 blocker such as ST-2427, ST-2578 and those disclosed in WO2010/129864, WO2015/157559, WO2017/059385, WO2018/183781, WO2018/183782, WO2020/072835, and/or WO2022/036297 the entire contents of each application hereby incorporated by reference.

In another embodiment, the additional therapeutic agent is ASP18071, CC-8464, ANP-230, ANP-231, NOC-100, NTX-1175, ASN008, NW3509, AM-6120, AM-8145, AM-0422, BL-017881, NTM-006, Opiranserin (Unafra™), brivoligide, SR419, NRD.E1, LX9211, LY3016859, ISC-17536, NFX-88, LAT-8881, AP-235, NYX 2925, CNTX-6016, S-600918, S-637880, RQ-00434739, KLS-2031, MEDI 7352, or XT-150.

In another embodiment, the additional therapeutic agent is Olinvyk, Zynrelef, Seglentis, Neumentum, Nevakar, HTX-034, CPL-01, ACP-044, HRS-4800, Tarlige, BAY2395840, LY3526318, Eliapixant, TRV045, RTA901, NRD1355-E1, MT-8554, LY3556050, AP-325, tetrodotoxin, Otenaproxesul, CFTX-1554, Funapide, iN1011-N17, JMKX000623/ODM-111, ETX-801, OLP-1002, ANP-230/DSP-2230, iN1011-N17, DSP-3905 or ACD440,

In another embodiment, the additional therapeutic agent is a sodium channel inhibitor (also known as a sodium channel blocker), such as the Na_{V}1.7 and Na_{V}1.8 blockers identified above.

The amount of additional therapeutic agent present in the compositions of this invention may be no more than the amount that would normally be administered in a composition comprising that therapeutic agent as the only active agent. The amount of additional therapeutic agent in the presently disclosed compositions may range from about 10% to 100% of the amount normally present in a composition comprising that agent as the only therapeutically active agent.

The compounds and salts of this invention or pharmaceutically acceptable compositions thereof may also be incorporated into compositions for coating an implantable medical device, such as prostheses, artificial valves, vascular grafts, stents and catheters. Accordingly, the invention, in another aspect, includes a composition for coating an implantable device comprising a compound or salt of the invention as described generally above, and in classes and subclasses herein, and a carrier suitable for coating said implantable device. In still another aspect, the invention includes an implantable device coated with a composition comprising a compound or salt of the invention as described generally above, and in classes and subclasses herein, and a carrier suitable for coating said implantable device. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

Another aspect of the invention relates to inhibiting Na_{V}1.8 activity in a biological sample or a subject, which method comprises administering to the subject, or contacting said biological sample with a compound of the invention, a pharmaceutically acceptable salt thereof, or a pharmaceutical composition thereof. The term “biological sample,” as used herein, includes, without limitation, cell cultures or extracts thereof; biopsied material obtained from a mammal or extracts thereof; and blood, saliva, urine, feces, semen, tears, or other body fluids or extracts thereof.

Inhibition of Na_{V}1.8 activity in a biological sample is useful for a variety of purposes that are known to one of skill in the art. Examples of such purposes include, but are not limited to, the study of sodium channels in biological and pathological phenomena; and the comparative evaluation of new sodium channel inhibitors.

### Synthesis of the Compounds of the Invention

The compounds of the invention can be prepared from known materials by the methods described in the Examples, other similar methods, and other methods known to one skilled in the art. As one skilled in the art would appreciate, the functional groups of the intermediate compounds in the methods described below may need to be protected by suitable protecting groups. Protecting groups may be added or removed in accordance with standard techniques, which are well-known to those skilled in the art. The use of protecting groups is described in detail in T. G. M. Wuts et al., Greene's Protective Groups in Organic Synthesis (4th ed. 2006).

### Radiolabeled Analogs of the Compounds of the Invention

In another aspect, the invention relates to radiolabeled analogs of the compounds of the invention. As used herein, the term “radiolabeled analogs of the compounds of the invention” refers to compounds that are identical to the compounds of the invention, as described herein, including all embodiments thereof, except that one or more atoms has been replaced with a radioisotope of the atom present in the compounds of the invention.

As used herein, the term “radioisotope” refers to an isotope of an element that is known to undergo spontaneous radioactive decay. Examples of radioisotopes include ³H, ¹⁴C, ³²P, ³⁵S, ¹⁸F, ³⁶Cl, and the like, as well as the isotopes for which a decay mode is identified in V. S. Shirley & C. M. Lederer, Isotopes Project, Nuclear Science Division, Lawrence Berkeley Laboratory, Table of Nuclides (January 1980).

The radiolabeled analogs can be used in a number of beneficial ways, including in various types of assays, such as substrate tissue distribution assays. For example, tritium (³H)- and/or carbon-14 (¹⁴C)-labeled compounds may be useful for various types of assays, such as substrate tissue distribution assays, due to relatively simple preparation and excellent detectability.

In another aspect, the invention relates to pharmaceutically acceptable salts of the radiolabeled analogs, in accordance with any of the embodiments described herein in connection with the compounds of the invention.

In another aspect, the invention relates to pharmaceutical compositions comprising the radiolabeled analogs, or pharmaceutically acceptable salts thereof, and a pharmaceutically acceptable carrier, adjuvant or vehicle, in accordance with any of the embodiments described herein in connection with the compounds of the invention.

In another aspect, the invention relates to methods of inhibiting voltage-gated sodium channels and methods of treating or lessening the severity of various diseases and disorders, including pain, in a subject comprising administering an effective amount of the radiolabeled analogs, pharmaceutically acceptable salts thereof, and pharmaceutical compositions thereof, in accordance with any of the embodiments described herein in connection with the compounds of the invention.

In another aspect, the invention relates to radiolabeled analogs, pharmaceutically acceptable salts thereof, and pharmaceutical compositions thereof, for use, in accordance with any of the embodiments described herein in connection with the compounds of the invention.

In another aspect, the invention relates to the use of the radiolabeled analogs, or pharmaceutically acceptable salts thereof, and pharmaceutical compositions thereof for the manufacture of medicaments, in accordance with any of the embodiments described herein in connection with the compounds of the invention.

In another aspect, the radiolabeled analogs, pharmaceutically acceptable salts thereof, and pharmaceutical compositions thereof, can be employed in combination therapies, in accordance with any of the embodiments described herein in connection with the compounds of the invention.

### EXAMPLES

### Abbreviations

Unless otherwise noted, or where the context dictates otherwise, the following abbreviations shall be understood to have the following meanings:

| Abbreviation | Meaning |
|---|---|
| NMR | Nuclear magnetic resonance |
| ESI-MS | Electrospray mass spectrometry |
| LC/MS | Liquid chromatography-mass spectrometry |
| UPLC | Ultra performance liquid chromatography |
| HPLC/MS/MS | High performance liquid chromatography/tandem mass spectrometry |
| IS | Internal standard |
| HPLC | High performance liquid chromatography |
| SFC | Supercritical fluid chromatography |
| ESI | Electrospray ionization |
| g | Grams |
| mg | Milligrams |
| kg | Kilograms |
| L | Liter(s) |
| mL | Milliliters |
| μL | Microliters |
| nL | Nanoliters |
| mol | Moles |
| mmol | Millimoles |
| hr, h | Hours |
| min | Minutes |
| ms | Millisecond |
| mm | Millimeters |
| μm | Micrometers |
| nm | Nanometer |
| MHz | Megahertz |
| Hz | Hertz |
| N | Normal (concentration) |
| M | Molar (concentration) |
| mM | Millimolar (concentration) |
| μM | Micromolar (concentration) |
| ppm | Parts per million |
| % w/v | Weight-volume concentration |
| % w/w | Weight-weight concentration |
| t-BuOH | Tert-butyl alcohol |
| CDI | 1,1′-Carbonyl diimidazole |
| DAST | Diethylaminosulfur trifluoride |
| DCM | Dichloromcthanc |
| DCE | Dichloroethane |
| DIEA, DIPEA | N, N-Diisopropyl ethyl amine |
| DMA | N,N-Dimethylacetamide |
| DMAP | N,N-Dimethylaminopyridine |
| DMF | N,N-Dimethylformamide |
| DMSO | Dimethyl sulfoxide |
| DRG | Dorsal root ganglia |
| EDC•HCl | Ethyl carbodiimide hydrochloride |
| EtOH | Ethanol |
| EtOAc | Ethyl acetate |
| HATU | 1-[Bis(dimethylamino)methylene]-1H-1,2,3-triazolo[4,5-b]pyridinium |
| | 3-Oxide hexafluorophosphate |
| HOBt | Hydroxybenzotriazole |
| EDCI | 1-Ethyl-3-(3-dimethylaminopropyl)carbodiimide |
| T3P | Propylphosphonic anhydride, i.e., 2,4,6-tripropyl-1,3,5,2,4,6- |
| | trioxatriphosphinane 2,4,6-trioxide |
| KOAc | Potassium acetate |
| m-CPBA | Meta-chloroperoxybenzoic acid |
| MeOH | Methanol |
| MTBE | Methyl tert-butyl ether |
| NaOH | Sodium hydroxide |
| NBS | N-Bromosuccinimide |
| NMP | N-Methylpyrrolidone |
| NMO | N-Methylmorpholine N-oxide |
| PdCl₂(dtbpf) | 1,1′-Bis(di-tert-butylphosphino)ferrocene palladium dichloride |
| PPTS | Pyridinium para-toluene sulfonate |
| TBAB | Tetra-n-butylammonium bromide |
| TBAF | Tetra-n-butylammonium fluoride |
| TBSCl | Tert-butyldimethylsilyl chloride |
| TBSOTf | Tert-butyldimethylsilyl trifluoromethanesulfonate |
| THF | Tetrahydrofuran |
| TEA | Triethylamine |
| TFA | Trifluoroacetic acid |
| RB | Round bottom (flask) |
| RT | Room temperature |
| ca. | Circa (approximately) |
| E-VIPR | Electrical stimulation voltage ion probe reader |
| HEK | Human embryonic kidney |
| KIR2.1 | Inward-rectifier potassium ion channel 2.1 |
| DMEM | Dulbecco's Modified Eagle's Medium |
| FBS | Fetal bovine serum |
| NEAA | Non-essential amino acids |
| HEPES | 2-[4-(2-Hydroxyethyl)piperazin-1-yl]ethanesulfonic acid |
| DiSBAC₆(3) | Bis-(1,3-dihexyl-thiobarbituric acid) trimethine oxonol |
| CC2-DMPE | Chlorocoumarin-2-dimyristoyl phosphatidylethanolamine |
| VABSC-1 | Voltage Assay Background Suppression Compound |
| HS | Human serum |
| BSA | Bovine Serum Albumin |

General methods. ¹H NMR spectra were obtained as solutions in an appropriate deuterated solvent such as dimethyl sulfoxide-d₆ (DMSO-d6).

Compound purity, retention time, and electrospray mass spectrometry (ESI-MS) data were determined by LC/MS analysis.

### LC/MS Methods

LC/MS determinations, unless otherwise specified, were carried out using one of the following chromatographic conditions:
1) Waters BEH C8 (1.7 μm, 2.1×50 mm) 2 to 98% acetonitrile in water (10 mM ammonium formate, pH 9), 45° C., flow rate 0.6 mL/min over 5.0 min;
2) Kinetex EVO C18 (2.6 μm, 2.1×50 mm) 2 to 98% acetonitrile in water (10 mM ammonium formate, pH 9), 45° C., flow rate 0.7 mL/min over 4.0 min;
3) Kinetex EVO C18 (2.6 μm 2.1×50 mm) 2 to 98% acetonitrile in water (10 mM ammonium formate, pH 9), 45° C., flow rate 1.0 mL/min over 1.5 min;
4) Waters Acquity UPLC BEH C18 (1.7 μm, 30×2.1 mm) 1 to 99% acetonitrile (0.035% TFA) in water (0.05% TFA), 60° C., flow rate=1.5 mL/min over 3 min;
5) Kinetex Polar C18 (2.6 μm, 3.0×50 mm) 5 to 95% acetonitrile in water (0.1% formic acid), flow rate 1.2 mL/min over 6 min;
6) SunFire C18 (3.5 μm, 75×4.6 mm) initial 5 to 95% acetonitrile in water (0.1% formic acid) for 1 min then linear gradient to 95% acetonitrile for 5 min. 45° C., flow rate 1.5 mL/min over 6 min;
7) XBridge C18 (5 μm, 4.6×75 mm) initial gradient 5 to 95% acetonitrile (NH₄HCO₃), 6 min run with 1 min equilibration gradient 0 to 3 min at 95% acetonitrile and hold for 3 min, flow rate 1.5 mL/min;
8) Waters CSH C18 (1.7 μm, 2.1×50 mm) 2 to 98% acetonitrile in water (0.1% TFA, pH 2), 45° C., flow rate 0.6 mL/min over 5.0 min;
9) Waters CSH C18 (1.7 μm, 2.1×50 mm) 2 to 95% acetonitrile in water (0.1% formic acid), 40° C., flow rate 0.8 mL/min over 4.6 min;
10) Waters BEH C18 (2.5 μm, 2.1×50 mm) 2 to 95% acetonitrile in water (0.1% NH₃), 40° C., flow rate 0.8 mL/min over 4.6 min;
11) Waters BEH C18 (3.5 μm, 75×4.6 mm) initial gradient 5 to 95% acetonitrile in water (0.1% formic acid) then linear gradient to 95% acetonitrile for 4 min, hold for 2 min at 95% acetonitrile, 45° C., flow rate 1.5 mL/min over 6 min;
12) Waters BEH C18 (2.5 Nm, 2.1×50 mm) 2 to 50% acetonitrile in water (0.1% NH₃), 40° C., flow rate 0.8 mL/min over 4.6 min;
13) Waters CSH C18 (1.7 μm, 2.1×50 mm) 2 to 98% acetonitrile in water (0.1% TFA), 45° C., flow rate 1.0 mL/min over 1.5 min;
14) Waters CSH C18 (1.7 μm, 2.1×50 mm) 2 to 95% acetonitrile in water (0.1% formic acid), 40° C., flow rate 0.8 mL/min over 1.4 min;
15) YMC Triart C18 (3 μm, 33×2.1 mm) 2 to 98% acetonitrile in water (5 mM NH₄OAc), flow rate 1.0 mL/min over 3 min;
16) Waters BEH C18 (2.5 μm, 2.1×50 mm) 2 to 95% acetonitrile in water (0.1% NH₃), 40° C., flow rate 0.8 mL/min over 1.4 min;
17) Waters Acquity UPLC BEH C18 (1.7 μm, 30×2.1 mm) 1 to 99% acetonitrile (0.035% TFA) in water (0.05% TFA), 60° C., flow rate=1.5 mL/min over 5 min;
18) Waters BEH C18 (2.5 μm, 2.1×50 mm) 20 to 70% acetonitrile in water (0.1% NH₃), 40° C., flow rate 0.8 mL/min over 4.60 min;
19) Kinetex Polar C18 (2.6 μm, 3.0×50 mm) 5 to 95% acetonitrile in water (0.1% formic acid), flow rate 1.2 mL/min over 3 min;
20) Waters Acquity UPLC BEH C18 column (1.7 μm, 30×2.1 mm) 1 to 99% acetonitrile (0.035% TFA) in water (0.05% TFA), 60° C., flow rate=1.5 mL/min over 1 min;
21) YMC Triart C18 (3 μm, 33×2.1 mm) 2 to 98% acetonitrile in water (0.05% formic acid), flow rate 1.0 mL/min over 3 min;
22) Waters Acquity UPLC BEH C18 (1.7 μm, 30×2.1 mm) 1 to 99% acetonitrile (0.05% ammonium formate) in water (0.05% ammonium formate), 60° C., flow rate=1.5 mL/min over 5 min;
23) Waters CSH C18 (1.7 μm, 2.1×50 mm) 2 to 98% acetonitrile in water (0.1% TFA), 45° C., flow rate 0.6 mL/min over 4.0 min;
24) Acquity BEH C8 (1.7 μm, 50×2.1 mm) 2 to 98% 90:10 acetonitrile:water (0.05% formic acid), flow rate 0.8 mL/min over 3 min;
25) XBridge C18 (5 μm, 50×4.6 mm) 10 to 90% acetonitrile in water (10 mM NH₄OAc), flow rate 1.2 mL/min over 6 min;
26) YMC Triart C18 (3 μm, 33×2.1 mm) 5 to 95% acetonitrile in water (0.05% formic acid), flow rate 1.0 mL/min over 12 min;
27) Waters BEH C8 (1.7 μm, 2.1×50 mm) 50 to 95% acetonitrile in water (0.1% NH₃), 40° C., flow rate 0.8 mL/min over 1.4 min.

### Example 1

### Preparation 1

### ethyl 4-benzyloxy-2-chloro-5-iodo-6-methyl-pyridine-3-carboxylate

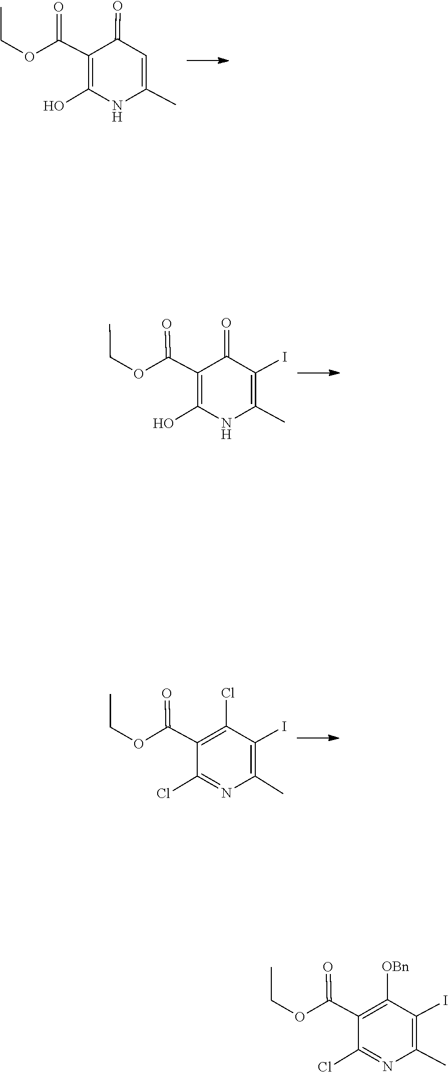 [SMILES: CCOC(=O)c1c(O)[nH]c(C)cc1=O ; CCOC(=O)c1c(O)[nH]c(C)c(I)c1=O ; CCOC(=O)c1c(Cl)nc(C)c(I)c1Cl ; CCOC(=O)c1c(Cl)nc(C)c(I)c1OCc1ccccc1]

### Step 1: ethyl 2-hydroxy-5-iodo-6-methyl-4-oxo-1H-pyridine-3-carboxylate

A suspension of ethyl 2-hydroxy-6-methyl-4-oxo-1H-pyridine-3-carboxylate (1.0 g, 5.1 mmol) and potassium carbonate (700 mg, 5.07 mmol) in water (10 mL) was heated to 100° C. Iodine (1.29 g, 5.08 mmol) was added portionwise over 10 min. After 30 min the reaction was cooled to room temperature and aqueous KHSO₄ was added. The resulting solid was collected by suction filtration and washed with 1:1 Et₂O/acetonitrile provided ethyl 2-hydroxy-5-iodo-6-methyl-4-oxo-1H-pyridine-3-carboxylate (1.43 g, 83%) as a white solid. ¹H NMR (400 MHz, CDCl₃) δ 4.44 (q, J=7.0 Hz, 2H), 2.57 (s, 3H), 1.42 (t, J=7.1 Hz, 3H). ESI-MS m/z calc. 322.97, found 323.84 (M+1)⁺.

### Step 2: ethyl 2,4-dichloro-5-iodo-6-methyl-pyridine-3-carboxylate

A suspension of ethyl 2-hydroxy-5-iodo-6-methyl-4-oxo-1H-pyridine-3-carboxylate (1.434 g, 4.217 mmol) in POCl₃ (16.5 g, 10 mL, 107 mmol) was heated at 120° C. for 2 h, concentrated and azeotroped with toluene twice. The residue was neutralized with a saturated aqueous solution of sodium bicarbonate and extracted with ethyl acetate (2×). The combined organics were washed with brine, dried over magnesium sulfate, filtered and concentrated under reduced pressure. Purification by silica gel chromatography (0-10% ethyl acetate/heptane) provided ethyl 2,4-dichloro-5-iodo-6-methyl-pyridine-3-carboxylate (1.15 g, 73%) as a white solid. ¹H NMR (400 MHz, CDCl₃) δ 4.46 (q, J=7.2 Hz, 2H), 2.83 (s, 3H), 1.40 (t, J=7.1 Hz, 3H). ESI-MS m/z calc. 358.90, found 359.79 (M+1)⁺.

### Step 3: ethyl 4-benzyloxy-2-chloro-5-iodo-6-methyl-pyridine-3-carboxylate

To a solution of benzyl alcohol (2.4 mL, 23 mmol) in THF (80 mL) and DMF (4 mL) at 0° C. was added sodium hydride (972 mg, 60% dispersion in mineral oil, 24.3 mmol) and the reaction mixture was stirred at room temperature for 30 min. Ethyl 2,4-dichloro-5-iodo-6-methyl-pyridine-3-carboxylate (8.3 g, 23 06 mmol) in THF (30 mL) was added at 0° C. and the reaction mixture was warmed to room temperature and stirred for 4 h. The reaction mixture was diluted with ethyl acetate (200 mL) and washed with water (3×200 mL) and brine (200 mL). The organic layer was dried over magnesium sulfate, filtered and concentrated in vacuo. The crude residue was purified by silica gel chromatography (0-10% ethyl acetate/heptane) to provide ethyl 4-benzyloxy-2-chloro-5-iodo-6-methyl-pyridine-3-carboxylate (4.205 g, 42%). ¹H NMR (500 MHz, DMSO-d₆) δ 7.50-7.47 (m, 2H), 7.46-7.38 (m, 3H), 5.10 (s, 2H), 4.35 (q, J=7.1 Hz, 2H), 2.71 (s, 3H), 1.26 (t, J=7.1 Hz, 3H). ESI-MS m/z calc. 430.98, found 432.0 (M+1)⁺.

### Preparation 2

### 4-benzyloxy-6-chloro-3-iodo-2-methyl-pyridine

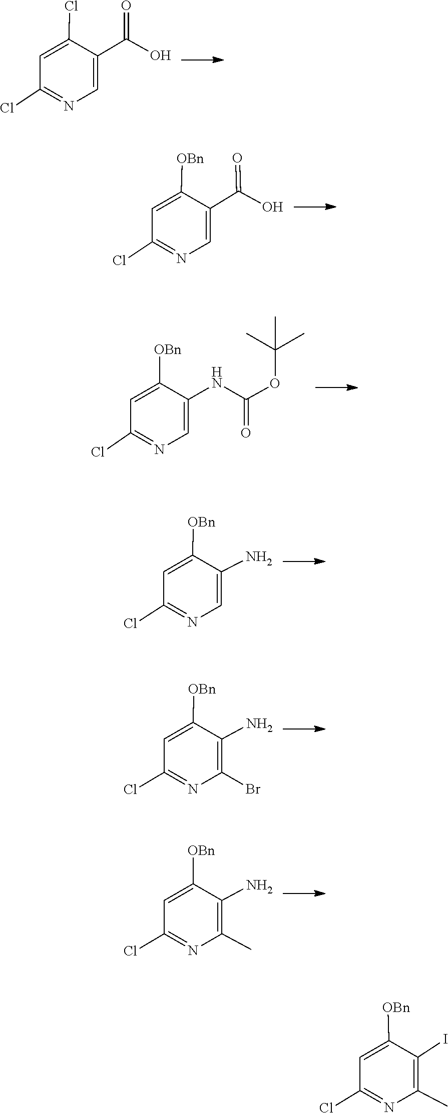

### Step 1: 4-benzyloxy-6-chloro-pyridine-3-carboxylic acid

Sodium hydride (50.0 g, 60% dispersion in mineral oil, 1.25 mol) was added in portions to a stirred solution of benzyl alcohol (140 g, 135 mL, 1.30 mol) in THF (500 mL) at 0° C. under argon. After 90 min at room temperature the reaction was cooled to 10° C. and a solution of 4,6-dichloropyridine-3-carboxylic acid (100 g, 469 mmol) in THF (500 mL) added over 30 min while maintaining reaction temperature below 30° C. After the addition, the reaction was stirred at room temperature for 4 h and then quenched by dropwise addition of water (1 L) at 0° C. The mixture was adjusted to pH 4 with aqueous 2 N HCl solution and extracted with ethyl acetate (2×500 mL). The combined organic extracts were concentrated and combined with toluene (500 mL). The mixture was filtered and the collected solids washed sequentially with toluene (500 mL) followed by heptane (500 mL) and then air dried to afford 4-benzyloxy-6-chloro-pyridine-3-carboxylic acid (120 g, 96%) as a beige solid. ¹H NMR (400 MHz, CDCl₃) δ 8.97 (d, J=8.2 Hz, 1H), 7.51-7.37 (m, 5H), 7.04 (s, 1H), 5.32 (d, J=17.4 Hz, 2H). ESI-MS m/z calc. 263.04, found 263.9 (M+1)⁺.

### Step 2: tert-butyl N-(4-benzyloxy-6-chloro-3-pyridyl)carbamate

DPPA (691 g, 540 mL, 2.51 mol) was added to a stirring suspension of 4-benzyloxy-6-chloro-pyridine-3-carboxylic acid (568 g, 2.05 mol), TEA (598 g, 823 mL, 5.91 mol) and tert-butanol (726 g, 937 mL, 9.80 mol) in toluene (8.5 L) The reaction was heated at 100° C. for 4 h then concentrated. The residue was partitioned between ethyl acetate (7.5 L) and saturated sodium hydrogen carbonate solution (5 L). Some insoluble material was removed by filtration and the organic phase separated and concentrated to afford tert-butyl N-(4-benzyloxy-6-chloro-3-pyridyl)carbamate (710 g, 93%) as a tan solid. ¹H NMR (400 MHz, CDCl₃) δ 8.98 (s, 1H), 7.47-7.35 (m, 5H), 6.84 (s, 1H), 6.74 (s, 1H), 5.14 (s, 2H), 1.51 (s, 9H). ESI-MS m/z calc. 334.11, found 335.02 (M+1)⁺.

### Step 3: 4-benzyloxy-6-chloro-pyridin-3-amine

To a suspension of tert-butyl N-(4-benzyloxy-6-chloro-3-pyridyl)carbamate (10.9 g, 29.3 mmol) in dichloromethane (25 mL) was added trifluoroacetic acid (37 g, 25 mL, 325 mmol). The resultant solution was stirred for 2.5 h then added portion-wise to saturated aqueous sodium carbonate solution (200 mL). The mixture was filtered and the solids washed with dichloromethane (50 mL). The organic phase was separated, dried over sodium sulfate and concentrated to a light brown solid. This was boiled in TBME (250 mL) then allowed to cool to room temperature. The mixture was filtered and the filtrate concentrated to a light brown solid. This was stirred with heptane (50 mL), filtered, washed with heptane (50 mL) and dried in air provided 4-benzyloxy-6-chloro-pyridin-3-amine (6 g, 84%) as a light brown solid. ¹H NMR (400 MHz, DMSO-d₆) δ 7.61 (t, J=7.6 Hz, 1H), 7.46 (d, J=6.9 Hz, 2H), 7.39-7.29 (m, 3H), 6.95 (s, 1H), 5.30-5.15 (m, 2H), 5.05 (d, J=27.9 Hz, 2H). ESI-MS m/z calc. 234.06, found 233.0 (M−1)⁻.

### Step 4: 4-benzyloxy-2-bromo-6-chloro-pyridin-3-amine

To a cooled solution of 4-benzyloxy-6-chloro-pyridin-3-amine (103.8 g, 437.9 mmol) in DCM (2 L) was added portion-wise NBS (84.3 g, 474 mmol) at 15-20° C. The mixture was stirred at room temperature for 30 min, then the solution washed with water (2×1 L) and dried over sodium sulfate. The reaction was repeated twice and all organics were combined and concentrated to a red solid. This was stirred in 1:4 ethyl acetate-heptane (1.5 L) for 1 h and the solid filtered, washed with 1:4 ethyl acetate-heptane (500 mL) then heptane (500 mL) and dried in air give 4-benzyloxy-2-bromo-6-chloro-pyridin-3-amine (411.6 g, 100%) as an orange solid. ¹H NMR (400 MHz, CDCl₃) δ 7.45-7.36 (m, 5H), 6.76 (s, 1H), 5.14 (d, J=16.9 Hz, 2H), 4.12 (t, J=7.1 Hz, 2H). ESI-MS m/z calc. 311.97, found 312.8 (M+1)⁺.

### Step 5: 4-benzyloxy-6-chloro-2-methyl-pyridin-3-amine

A mixture of 4-benzyloxy-2-bromo-6-chloro-pyridin-3-amine (10.0 g, 31.8 mmol), trimethyl boroxine (4.8 g, 38 mmol), potassium carbonate (8.80 g, 63.7 mmol) in 1,4-dioxane (100 mL) and water (10 mL) was degassed with argon then treated with Pd(PPh₃)₄ (1.8 g, 1.6 mmol). The reaction mixture was heated under reflux for 7 h then cooled to room temperature and partitioned between ethyl acetate (300 mL) and water (100 mL). The organic phase was dried over sodium sulfate and concentrated to a dark oil. Purification by silica gel chromatography (0-25% ethyl acetate/heptane) gave 4-benzyloxy-6-chloro-2-methyl-pyridin-3-amine (5 g, 63%) as a light brown solid. ¹H NMR (400 MHz, CDCl₃) δ 7.40 (t, J=3.7 Hz, 5H), 6.72 (d, J=6.4 Hz, 1H), 5.12 (d, J=23.4 Hz, 2H), 3.71 (s, 2H), 2.37 (s, 3H). ESI-MS m/z calc. 248.07, found 248.97 (M+1)⁺.

### Step 6: 4-benzyloxy-6-chloro-3-iodo-2-methyl-pyridine

To a cooled solution of 4-benzyloxy-6-chloro-2-methyl-pyridin-3-amine (25.0 g, 94.8 mmol) in 16% aqueous hydrochloric acid (250 mL) was added dropwise a solution of sodium nitrite (9.80 g, 142 mmol) in water (40 mL) at 0-2° C. After stirring at 0-2° C. for 1 minute the solution was added to a mixture of sodium iodide (71.1 g, 474 mmol), water (250 mL) and dichloromethane (250 mL) at 0° C. After stirring at 0-2° C. for 10 min the mixture was allowed to warm to room temperature over 30 min. The organic phase was separated, dried over sodium sulfate and concentrated. Purification by silica gel chromatography (0-20% ethyl acetate/heptane) followed by trituration with heptane provided 4-benzyloxy-6-chloro-3-iodo-2-methyl-pyridine (13.0 g, 35%). ESI-MS m/z calc. 358.96, found 359.95 (M+1)⁺. ¹H NMR (400 MHz, CDCl₃) δ 7.47-7.34 (m, 5H), 6.61 (s, 1H), 5.23-5.19 (m, 2H), 2.77-2.73 (m, 3H).

### Preparation 3

### 4-benzyloxy-6-chloro-2-methyl-pyridine-3-carboxylic acid

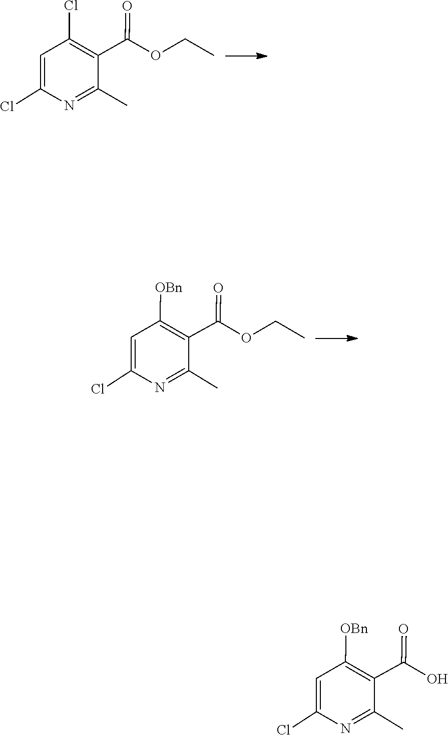 [SMILES: CCOC(=O)c1c(Cl)cc(Cl)nc1C ; CCOC(=O)c1c(OCc2ccccc2)cc(Cl)nc1C ; Cc1nc(Cl)cc(OCc2ccccc2)c1C(=O)O]

### Step 1: ethyl 4-benzyloxy-6-chloro-2-methyl-pyridine-3-carboxylate

A mixture of ethyl 4,6-dichloro-2-methyl-pyridine-3-carboxylate (20.0 g, 85.4 mmol) and benzyl alcohol (10.0 g, 92.5 mmol) in DMF (200 mL) was treated with potassium tert-butoxide (12.0 g, 107 mmol) and stirred at room temperature for 16 h. The mixture was diluted with water (500 mL) and extracted with ethyl acetate (2×400 mL). The combined organic layers were dried over magnesium sulfate, filtered and concentrated. Purification by silica gel chromatography (5-8% ethyl acetate/hexane) provided ethyl 4-benzyloxy-6-chloro-2-methyl-pyridine-3-carboxylate (12 g, 26%). ¹H NMR (400 MHz, CDCl₃) δ 7.43-7.28 (m, 5H), 6.77 (s, 1H), 5.13 (s, 2H), 4.36 (q, J=7.1 Hz, 2H), 2.48 (s, 3H), 1.30 (t, J 7.1 Hz, 3H). ESI-MS m/z calc. 305.08, found 306.14 (M+1)⁺.

### Step 2: 4-benzyloxy-6-chloro-2-methyl-pyridine-3-carboxylic acid

To a solution of ethyl 4-benzyloxy-6-chloro-2-methyl-pyridine-3-carboxylate (1.0 g, 3.2 mmol) in THF (10 mL) and methanol (10 mL) were added a solution of lithium hydroxide monohydrate (700 mg, 16.7 mmol) in water (10 mL) at room temperature and the mixture was stirred at room temperature for 19 h, followed by heating at 50° C. for 2 h. An additional portion of lithium hydroxide monohydrate (700 mg, 16.7 mmol) was added at room temperature and the mixture stirred at 50° C. for 4 days. The mixture was concentrated, dissolved in water (20 mL) and acidified with a 1 M aqueous HCl until pH 3-4. The precipitate was filtered and rinsed with water (50 mL). The solid was dried under high vacuum to provide 4-benzyloxy-6-chloro-2-methyl-pyridine-3-carboxylic acid (1 g, 104%) as a white solid. ¹H NMR (400 MHz, DMSO-d₆) δ 13.49 (br s, 1H), 7.44-7.32 (m, 5H), 7.23 (s, 1H), 5.29 (s, 2H), 2.37 (s, 3H). ESI-MS m/z calc. 277.05, found 278.2 (M+1)⁺.

### Intermediate A-1

### 4-benzyloxy-2-bromo-3,5,6-trimethyl-pyridine

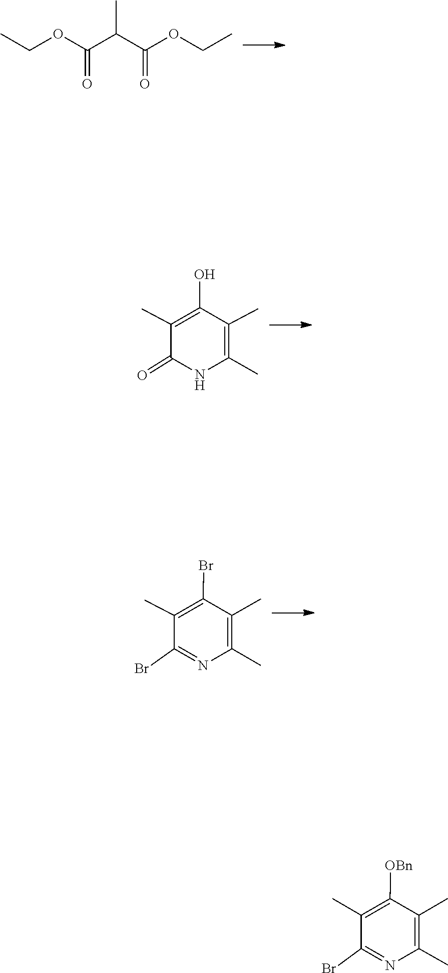

### Step 1: 4-hydroxy-3,5,6-trimethyl-1H-pyridin-2-one

Diethyl 2-methylpropanedioate (21.90 g, 125.7 mmol) was dissolved in toluene (5 mL) and sodium ethoxide (40.7 mL of 21% w/v, 126 mmol) in ethanol was added. The reaction was stirred at room temperature for 1 h. Ethyl 3-amino-2-methyl-but-2-enoate (18.0 g, 126 mmol) was added and the reaction heated to reflux for 18 h. The condenser was left open to the air at reflux for an additional 6 h.

The reaction was then allowed to cool to room temperature, diluted with water (100 mL) and stirred for 1 h. The mixture was partitioned between water and toluene. The aqueous layer was washed with toluene (2×50 mL) and the aqueous layer pH adjusted to pH 5. The resulting precipitate was filtered and dried under vacuum to provide 4-hydroxy-3,5,6-trimethyl-1H-pyridin-2-one (3 g, 16%) as an off-white solid.

¹H NMR (400 MHz, DMSO-d₆) δ 10.80 (s, 1H), 9.11 (s, 1H), 2.08 (s, 3H), 1.85 (s, 3H), 1.82 (s, 3H).

### Step 2: 2,4-dibromo-3,5,6-trimethyl-pyridine

4-Hydroxy-3,5,6-trimethyl-1H-pyridin-2-one (3 g, 19.59 mmol) and POBr₃ (12.0 g, 41.9 mmol) were combined in toluene (2 mL) and heated to 110° C. for 18 h. The reaction was allowed to cool to room temperature then poured into ice water. The resulting solid was filtered to provide 2,4-dibromo-3,5,6-trimethyl-pyridine (2 g, 37%). ¹H NMR (400 MHz, DMSO-d₆) δ 2.50 (dd, J=3.7, 1.9 Hz, 3H), 2.47 (s, 3H), 2.33 (s, 3H). ESI-MS m/z calc. 278.97, found 279.9 (M+1)⁺.

### Step 3: 4-benzyloxy-2-bromo-3,5,6-trimethyl-pyridine

4-Benzyloxy-2-bromo-3,5,6-trimethyl-pyridine was prepared from 2,4-dibromo-3,5,6-trimethyl-pyridine and benzyl alcohol using a procedure analogous to that found in Preparation 1, step 3. H NMR (500 MHz, DMSO-d₆) δ 7.50-7.34 (m, 5H), 4.86 (s, 2H), 2.37 (s, 3H), 2.21 (s, 3H), 2.12 (s, 3H). ESI-MS m/z calc. 305.04, found 306.0 (M+1)⁺.

### Intermediate A-2

### 4-(benzyloxy)-2-bromo-3-methoxy-6-methylpyridine

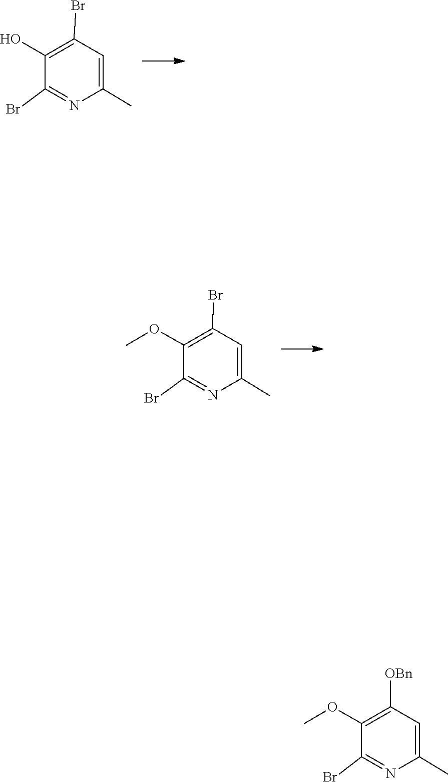

### Step 1: 2,4-dibromo-3-methoxy-6-methylpyridine

In a 2 L round bottomed flask immersed in a water bath, iodomethane (37.61 g, 16.50 mL, 265 mmol) was slowly added via syringe (no exotherm) to a stirred suspension of 2,4-dibromo-6-methylpyridin-3-ol (50 g, 178.0 mmol) and potassium carbonate (36.90 g, 267 mmol) in acetone (1.24 L). The suspension was stirred over the weekend at room temperature. The reaction mixture was filtered, and the filtered cake was washed with acetone. The brown filtrate was collected and concentrated in vacuo at 40° C. The residue was partitioned between MTBE (400 mL) and water (400 mL) and stirred at room temperature. The solid was filtered and rinsed with water (200 mL) provided 2,4-dibromo-3-methoxy-6-methylpyridine (49.72 g, 90%). ¹H NMR (400 MHz, DMSO-d₆) δ 7.67 (s, 1H), 3.82 (s, 3H), 2.41 (s, 3H). ESI-MS m/z calc. 278.89, found 279.8 (M+1)⁺.

### Step 2: 4-(benzyloxy)-2-bromo-3-methoxy-6-methylpyridine

NaH (523 mg, 60% dispersion in mineral oil, 13.076 mmol) was added to a stirred solution of benzyl alcohol (1.35 g, 12.48 mmol) in DMF (24 mL) at −10° C. and the mixture was stirred for 1 h. A solution of 2,4-dibromo-3-methoxy-6-methylpyridine (3.5 g, 12.44 mmol) in DMF (5 mL) was added. The mixture was stirred at −10° C. for 1 h then allowed to warm to room temperature over 30 min. The mixture was stirred at room temperature for a further 1 h. The mixture was partitioned between water (75 mL) and ethyl acetate (100 mL) and the layers were separated. The organic phase was washed with water (3×75 mL) and brine, dried over magnesium sulfate, filtered and concentrated in vacuo. Purification by silica gel chromatography (120 g silica, 0 to 40% ethyl acetate/heptane) gave 4-(benzyloxy)-2-bromo-3-methoxy-6-methylpyridine (2.25 g, 58%) as a white solid. ¹H NMR (400 MHz, CDCl₃) δ 7.42-7.33 (m, 5H), 6.69 (s, 1H), 5.14 (s, 2H), 3.85 (s, 3H), 2.43 (s, 3H). ESI-MS m/z calc. 307.02, found 308.0 (M+1)⁺.

The following Intermediates were prepared using a procedure analogous to that found in

Intermediate A-2 using 2-iodopropane and 2-bromoethyl methyl ether in step 1.

| | | MW & | |
|---|---|---|---|
| | Compound | Found | |
| Intermediate | Name | [M + H]⁺ | NMR (shifts in ppm) |
| Intermediate A - | 4-benzyloxy-2- | 335.05, | ¹H NMR (400 MHz, CDCl₃) δ 7.42-7.34 (m, |
| 3 | bromo-3- | 336.1 | 5H), 6.69 (s, 1H), 5.11 (s, 2H), 4.57-4.48 (m, |
| | isopropoxy-6- | | 1H), 2.46 (s, 3H), 1.32 (d, J = 1.4 Hz, 3H), |
| | methyl-pyridine | | 1.30 (d, J = 1.8 Hz, 3H) |
| Intermediate A - | 4-benzyloxy-2- | 351.05, | ¹H NMR (400 MHz, CDCl₃) δ 7.43-7.32 (m, |
| 4 | bromo-3-(2- | 352.1 | 5H), 6.69 (s, 1H), 5.14 (s, 2H), 4.16 (t, J = 4.8 |
| | methoxyethoxy)- | | Hz, 2H), 3.72 (t, J = 4.8 Hz, 2H), 3.38 (s, 3H), |
| | 6-methyl- | | 2.44 (s, 3H) |
| | pyridine | | |

### Intermediate A-5

### Ethyl 4-(benzyloxy)-2-chloro-5-methoxy-6-methylnicotinate

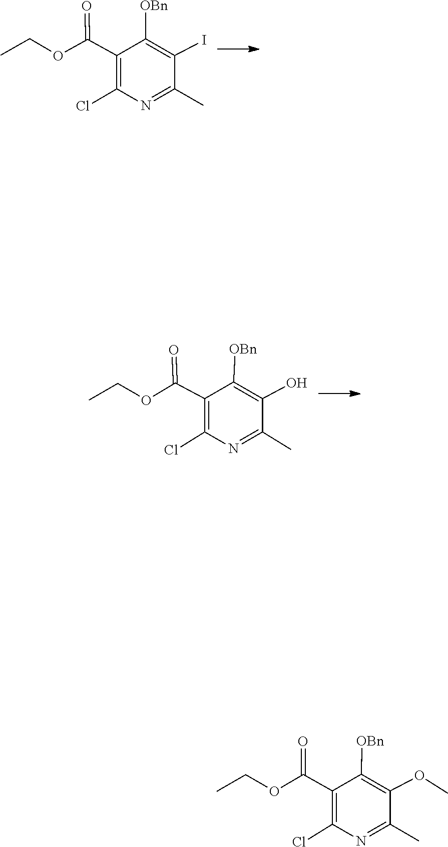 [SMILES: CCOC(=O)c1c(Cl)nc(C)c(I)c1OCc1ccccc1 ; CCOC(=O)c1c(Cl)nc(C)c(O)c1OCc1ccccc1 ; CCOC(=O)c1c(Cl)nc(C)c(OC)c1OCc1ccccc1]

### Step 1: ethyl 4-(benzyloxy)-2-chloro-5-hydroxy-6-methylnicotinate

A solution of iPrMgCl (544 mL, 2.0 M solution in Et₂O, 1.1 mol) was added to a stirring solution of ethyl 4-(benzyloxy)-2-chloro-5-iodo-6-methylnicotinate (Preparation 1, 414 g, 906 mmol) in Et₂O (5 L) at −11 to −5° C. over 40 min. The reaction mixture was stirred at −10° C. for 10 min. Trimethyl borate (188 g, 1.81 mol) was added at −10° C. over 10 min. The reaction was warmed to room temperature and stirred for 1 h. The mixture was quenched by addition of a mixture of ammonium chloride (2 M, 1.5 L) and brine (1.5 L). The organic phase was separated, dried over sodium sulfate, filtered and concentrated in vacuo provided (4-(benzyloxy)-6-chloro-5-(ethoxycarbonyl)-2-methylpyridin-3-yl)boronic acid (395 g, 94%) as a light yellow solid.

The crude boronic acid was suspended in acetonitrile (4 L) and a solution of Oxone (418 g, 680 mmol) in water (2 L) was added at 0° C. The reaction mixture was warmed to room temperature and stirred for 16 h. Additional Oxone (60 g, 98 mmol) was added and the reaction stirred at room temperature for a further 20 h. The mixture was filtered and the solid rinsed with acetonitrile (2×250 mL). The solid was partitioned between brine (1 L) and ethyl acetate (2 L). The organic phase was separated, dried over sodium sulfate and concentrated in vacuo. Heptane (3 L) was added and the mixture was concentrated to around 1.5 kg weight, then cooled to room temperature and filtered. The solid was dissolved in MTBE (4 L) and washed with 1 M NaOH (2×1 L). The combined aqueous extracts were acidified to pH 2 by addition of 3 N HCl and extracted with ethyl acetate (2×1 L). The organic extracts were combined, dried over sodium sulfate, filtered and concentrated in vacuo to around 750 g. Heptane (1.5 L) was added and the mixture was concentrated to around 1.5 kg inducing crystallization of the product. Heptane (1.5 L) was added and the mixture was concentrated to around 1.5 kg. The solid was filtered, rinsed with heptane (200 mL) and dried in vacuo to provide ethyl 4-(benzyloxy)-2-chloro-5-hydroxy-6-methylnicotinate (209 g, 69%). ¹H NMR (400 MHz, DMSO-d₆) δ 9.87 (s, 1H), 7.42-7.31 (m, 5H), 5.16 (s, 2H), 4.23 (q, J=7.1 Hz, 2H), 2.38 (s, 3H), 1.18 (t, J=7.1 Hz, 3H). ESI-MS m/z calc. 321.08, found 322.1 (M+1)⁺.

### Step 2: ethyl 4-(benzyloxy)-2-chloro-5-methoxy-6-methylnicotinate

Cesium carbonate (125 g, 384 mmol) and methyl iodide (43 g, 18.9 mL, 303 mmol) were successively added to a solution of ethyl 4-(benzyloxy)-2-chloro-5-hydroxy-6-methylnicotinate (83 g, 255 mmol) in acetonitrile (415 mL) at room temperature. The reaction mixture was stirred for 1 h and then filtered. The cake was rinsed with acetonitrile (2×100 mL) and the filtrates concentrated in vacuo. The residue was solubilized in DCM (100 mL), filtered through a plug of silica (75 g) and rinsed with DCM (1.5 L). The filtrates were concentrated in vacuo to provide ethyl 4-(benzyloxy)-2-chloro-5-methoxy-6-methylnicotinate (81.4 g, 94%). ¹H NMR (400 MHz, DMSO-d₆) δ 7.48-7.28 (m, 5H), 5.24 (s, 2H), 4.26 (q, J=7.3 Hz, 2H), 3.82 (s, 3H), 2.42 (s, 3H), 1.19 (t, J=7.1 Hz, 3H). ESI-MS m/z calc. 335.09, found 336.1 (M+1)⁺.

### Intermediate A-6

### methyl 4-benzyloxy-2-chloro-5-methoxy-6-methyl-pyridine-3-carboxylate

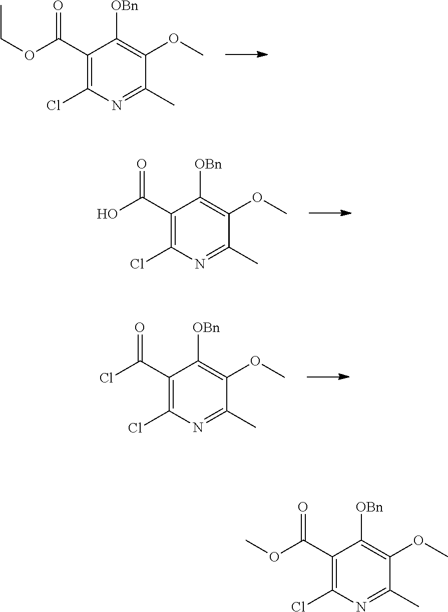

### Step 1: 4-benzyloxy-2-chloro-5-methoxy-6-methyl-pyridine-3-carboxylic acid

A solution of ethyl 4-benzyloxy-2-chloro-5-methoxy-6-methyl-pyridine-3-carboxylate (490 mg, 1.39 mmol) in methanol (5 mL) and THF (3 mL) was treated with aqueous NaOH (3 mL of 1 M, 3 mmol) and stirred for 12 h at 65° C. The mixture was quenched with 1 N HCl solution, diluted with ethyl acetate, washed with a saturated aqueous ammonium chloride and brine. The organic layer was dried over magnesium sulfate, filtered, and concentrated under reduced pressure. The resulting solid was triturated in 1:2 dichloromethane/hexanes and filtered to provide 4-benzyloxy-2-chloro-5-methoxy-6-methyl-pyridine-3-carboxylic acid. ¹H NMR (400 MHz, DMSO-d₆) δ 13.88 (br s, 1H), 7.63-7.18 (m, 5H), 5.22 (s, 2H), 3.81 (s, 3H), 2.40 (s, 3H). ESI-MS m/z calc. 307.06, found 308.2 (M+1)⁺.

### Step 2: 4-benzyloxy-2-chloro-5-methoxy-6-methyl-pyridine-3-carbonyl chloride

A vial was charged with 4-benzyloxy-2-chloro-5-methoxy-6-methyl-pyridine-3-carboxylic acid from step 1. DCM (10 mL) was added and the resulting slurry was cooled to 0° C. A solution of (COCl)₂ in DCM (1.5 mL of 2 M, 3 mmol) was added followed by DMF (10 μL, 0.13 mmol). The resulting mixture was stirred for 30 min at 0° C., then concentrated to provide 4-benzyloxy-2-chloro-5-methoxy-6-methyl-pyridine-3-carbonyl chloride. ESI-MS m/z calc. 325.03, found 326.2 (M+1)⁺.

### Step 3: methyl 4-benzyloxy-2-chloro-5-methoxy-6-methyl-pyridine-3-carboxylate

A solution of 4-benzyloxy-2-chloro-5-methoxy-6-methyl-pyridine-3-carbonyl chloride from step 2 in DCM (5 mL) at 0° C. was treated with DIPEA (315 μL, 1.81 mmol), DMAP (17 mg, 0.14 mmol) and methanol (1 mL, 25 mmol). The resulting mixture was stirred for 30 min at room temperature, then diluted with methylene chloride, washed with a saturated solution of aqueous ammonium chloride and then brine. The organic layer was dried over magnesium sulfate, filtered, and concentrated under reduced pressure. Purification by silica gel chromatography (0-70% ethyl acetate/hexanes over 20 min) provided methyl 4-benzyloxy-2-chloro-5-methoxy-6-methyl-pyridine-3-carboxylate (346.1 mg, 73%). ¹H NMR (400 MHz, DMSO-d₆) δ 7.46-7.32 (m, 5H), 5.23 (s, 2H), 3.82 (s, 3H), 3.79 (s, 3H), 2.42 (s, 3H). ESI-MS m/z calc. 321.08, found 322.256 (M+1)⁺.

### Intermediate A-7

### 6-Bromo-4-((4-methoxybenzyl)oxy)-2,5-dimethylnicotinonitrile

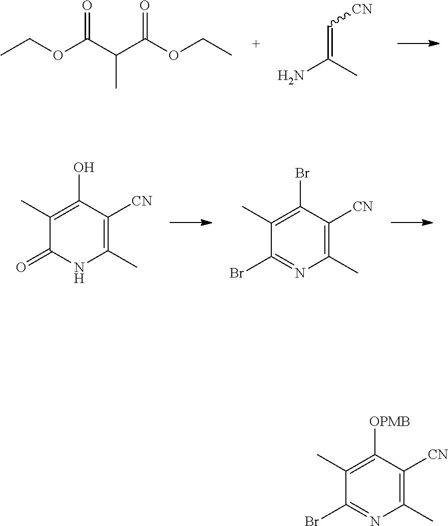

### Step 1: 4-hydroxy-2,5-dimethyl-6-oxo-1,6-dihydropyridine-3-carbonitrile

Diethyl 2-methylmalonate (10.3 g, 59.1 mmol) and 3-aminocrotononitrile (1.8 g, 22 mmol) were combined in a sealed tube and heated at 200° C. for 165 min. The reaction mixture was cooled to room temperature and treated with MTBE. The resulting precipitate was filtered and dried provided 4-hydroxy-2,5-dimethyl-6-oxo-1,6-dihydropyridine-3-carbonitrile (1.3 g, 36%). ESI-MS m/z calc. 164.06, found 165.2 (M+1)⁺.

### Step 2: 4,6-dibromo-2,5-dimethylnicotinonitrile

POBr₃ (5.0 g, 17 mmol) was added to a stirring mixture of 4-hydroxy-2,5-dimethyl-6-oxo-1,6-dihydropyridine-3-carbonitrile (1.3 g, 7.9 mmol) in acetonitrile (15 mL). The reaction mixture was stirred at reflux under nitrogen for 4 h and 45 min. The mixture was concentrated in vacuo and partitioned between ethyl acetate and a saturated sodium bicarbonate solution. The mixture was filtered to provide a first crop of product (250 mg). The organic layer was separated, dried over magnesium sulfate, filtered and concentrated in vacuo to provide a second crop of product. The two crops were combined to provide 4,6-dibromo-2,5-dimethylnicotinonitrile (1.54 g, 67%). ESI-MS m/z calc. 287.89, found 288.8 (M+1)⁺.

### Step 3: 6-bromo-4-((4-methoxybenzyl)oxy)-2,5-dimethylnicotinonitrile

6-Bromo-4-((4-methoxybenzyl)oxy)-2,5-dimethylnicotinonitrile was prepared from 4,6-dibromo-2,5-dimethylnicotinonitrile and (4-methoxyphenyl)methanol using a procedure analogous to that found in Preparation 1, step 3 using 2-MeTHF as the solvent. ESI-MS m/z calc. 346.03, found 224.9 (M-PMB)⁺.

### Intermediate A-8

### 4-(Benzyloxy)-2-chloropyridine

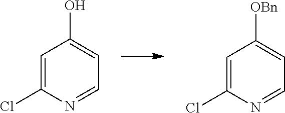 [SMILES: Oc1ccnc(Cl)c1 ; Clc1cc(OCc2ccccc2)ccn1]

Cesium carbonate (1.89 g, 5.80 mmol) and benzyl bromide (700 μL, 5.89 mmol) were successively added to a solution of 2-chloropyridin-4-ol (500 mg, 3.86 mmol) in acetonitrile (8 mL). The reaction mixture was stirred at room temperature for 1 h. The mixture was diluted with ethyl acetate (15 mL) and poured over a saturated aqueous sodium bicarbonate solution (20 mL). The aqueous layer was separated and extracted with ethyl acetate (2×20 mL). The combined organic extracts were washed with brine (30 mL), dried over magnesium sulfate and concentrated in vacuo. Purification by silica gel chromatography (24 g silica, 0-100% ethyl acetate/heptane) gave 4-(benzyloxy)-2-chloropyridine (601 mg, 71%) as a crystalline white solid. ¹H NMR (500 MHz, CDCl₃) δ 8.20 (d, J=5.8 Hz, 1H), 7.44-7.35 (m, 5H), 6.92 (d, J=2.2 Hz, 1H), 6.82 (dd, J=5.8, 2.2 Hz, 1H), 5.11 (s, 2H). ESI-MS m/z calc. 219.05, found 220.3 (M+1)⁺; 218.1 (M−1)⁻.

### Intermediate A-9

### 4-benzyloxy-6-chloro-N,N-dimethyl-pyridin-2-amine

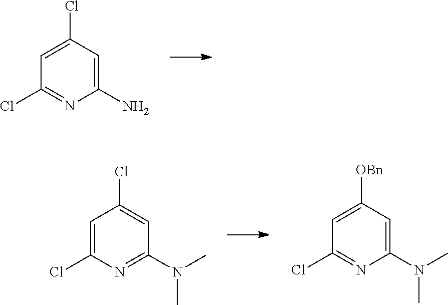 [SMILES: Nc1cc(Cl)cc(Cl)n1 ; CN(C)c1cc(Cl)cc(Cl)n1 ; CN(C)c1cc(OCc2ccccc2)cc(Cl)n1]

### Step 1: 4,6-dichloro-N,N-dimethyl-pyridin-2-amine

A solution of 4,6-dichloropyridin-2-amine (300 mg, 1.84 mmol) in THF (5 mL) was treated with sodium hydride (300 mg, 60% dispersion in mineral oil, 7.50 mmol) at 0° C. Methyl iodide (732 mg, 5.16 mmol) was added and the mixture was stirred at room temperature overnight. The reaction mixture was quenched with water and extracted with ethyl acetate. The organic layer was dried over sodium sulfate, evaporated and purified by silica gel column chromatography (0-30% ethyl acetate/hexanes) to provide 4,6-dichloro-N,N-dimethyl-pyridin-2-amine (220 mg, 63%). ESI-MS m/z calc. 190.01, found 191.1 (M+1)⁺. ¹H NMR (400 MHz, CDCl₃) δ 6.55 (d, J=1.3 Hz, 1H), 6.34 (d, J=1.3 Hz, 1H), 3.07 (s, 6H).

### Step 2: 4-benzyloxy-6-chloro-N,N-dimethyl-pyridin-2-amine

4-Benzyloxy-6-chloro-N,N-dimethyl-pyridin-2-amine was prepared from 4,6-dichloro-N,N-dimethyl-pyridin-2-amine and benzyl alcohol using a procedure analogous to that found in Preparation 1, step 3. ESI-MS m/z calc. 262.09, found 263.3 (M+1)⁺. ¹H NMR (400 MHz, CDCl₃) δ 7.41-7.32 (m, 5H), 6.26 (d, J=1.7 Hz, 1H), 5.89 (d, J=1.8 Hz, 1H), 5.05 (s, 2H), 3.03 (s, 6H).

### Intermediate A-10

### 4-benzyloxy-2-chloro-N,N,6-trimethyl-pyridin-3-amine

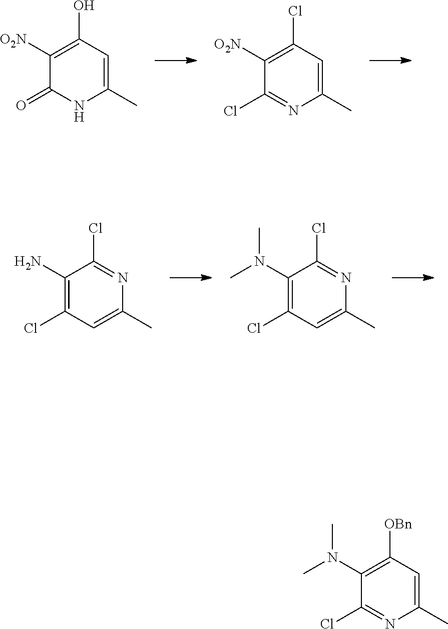

### Step 1: 2,4-dichloro-6-methyl-3-nitro-pyridine

A solution of 4-hydroxy-6-methyl-3-nitro-1H-pyridin-2-one (5.0 g, 28.8 mmol), diethyl aniline (4.1 g, 4.5 mL, 27 mmol) and POCl₃ (39.5 g, 24.5 mL, 252 mmol) was stirred at room temperature for 10 min and then stirred at 120° C. for 12 h. The mixture was cooled, diluted with ice-cold water and stirred for 1.5 h. The aqueous layer was extracted with ethyl acetate. The organic layer was washed with brine, dried over sodium sulfate and concentrated under reduced pressure to afford 2,4-dichloro-6-methyl-3-nitro-pyridine (4.5 g, 72%). ¹H NMR (400 MHz, CDCl₃) δ 7.29 (s, 1H), 2.61 (s, 3H). ESI-MS m/z calc. 205.97, found 206.80 (M+1)⁺.

### Step 2: 2,4-dichloro-6-methyl-pyridin-3-amine

A mixture of 2,4-dichloro-6-methyl-3-nitro-pyridine (3.0 g, 13.9 mmol) and iron (20 g, 350 mmol) in methanol (60 mL) and water (15 mL) was added ammonium chloride (743 mg, 13.6 mmol) at 0° C. and stirred for 10 min. The resulting reaction mixture was heated to 80° C. and stirred for 5 h. The reaction mixture was filtered. The filtrate was diluted with water and extracted with ethyl acetate. The organic layer was washed with brine, dried over sodium sulfate, filtered and concentrated under reduced pressure to afford 2,4-dichloro-6-methyl-pyridin-3-amine (2.2 g, 89%). ¹H NMR (400 MHz, CDCl₃) δ 7.02 (s, 1H), 4.29 (s, 2H), 2.41 (s, 3H). ESI-MS m/z calc. 175.99, found 177.0 (M+1)⁺.

### Step 3: 2,4-dichloro-N,N,6-trimethyl-pyridin-3-amine

To a solution of 2,4-dichloro-6-methyl-pyridin-3-amine (1.02 g, 5.76 mmol) in THF (28 mL) at 0° C. was added potassium tert-butoxide in THF (17.3 mL of 1.0 M, 17.3 mmol) and the mixture shirred for 30 min. Methyl iodide (4.1 g, 1.8 mL, 29 mmol) was added dropwise and the reaction allowed to warm to room temperature and stirred for 1 h. The reaction was quenched with half-saturated brine (50 mL) and the mixture extracted with ethyl acetate (2×50 mL). The combined extracts were washed with brine (50 mL), dried over sodium sulfate and concentrated in vacuo. Purification by silica gel chromatography (5-20% ethyl acetate/heptanes) provided 2,4-dichloro-N,N,6-trimethyl-pyridin-3-amine (668 mg, 57%). ¹H NMR (400 MHz, CDCl₃) δ 7.09 (s, 1H), 2.84 (s, 6H), 2.45 (s, 3H). ESI-MS m/z calc. 204.02, found 205.07 (M+1)⁺.

### Step 4: 4-benzyloxy-2-chloro-N,N,6-trimethyl-pyridin-3-amine

4-Benzyloxy-2-chloro-N,N,6-trimethyl-pyridin-3-amine was prepared from 2,4-dichloro-N,N,6-trimethyl-pyridin-3-amine and benzyl alcohol using a procedure analogous to that found in Preparation 1, step 3. ¹H NMR (400 MHz, CDCl₃) δ 7.42-7.34 (m, 5H), 6.66 (s, 1H), 5.12 (s, 2H), 2.79 (s, 6H), 2.42 (s, 3H). ESI-MS m/z calc. 276.10, found 276.95 (M+1)⁺.

### Intermediate A-11

### methyl 4-benzyloxy-6-chloro-5-(dimethylamino)-2-methyl-pyri dime-3-carboxylate

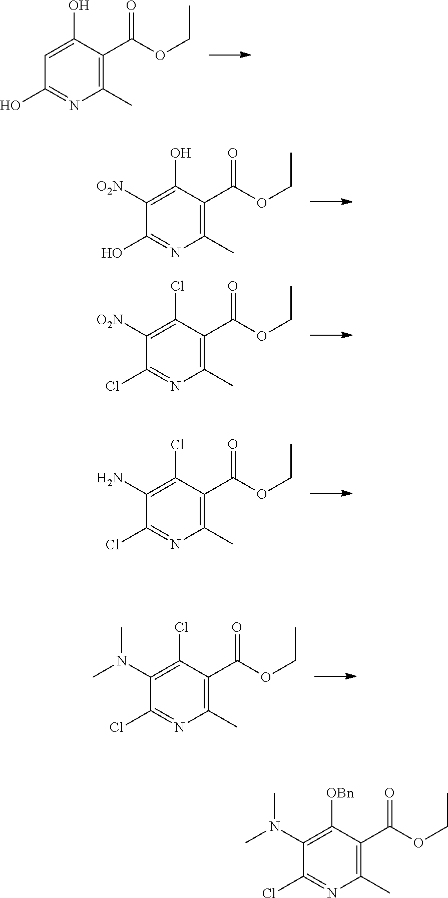

### Step 1: ethyl 4,6-dihydroxy-2-methyl-5-nitro-pyridine-3-carboxylate

To a solution of ethyl 4,6-dihydroxy-2-methyl-pyridine-3-carboxylate (10.0 g, 48.9 mmol) in sulfuric acid (75 mL) at 0° C. was added dropwise nitric acid (4.2 g, 3.0 mL, 67 mmol) over 15 min. The reaction mixture was then stirred at 0° C. for 2 h. It was then poured onto crushed ice (200 g) and vigorously stirred overnight at room temperature. The precipitate was filtered, rinsed with cold water (2×150 mL), washed with heptanes (3×150 mL) and air-dried to provide ethyl 4,6-dihydroxy-2-methyl-5-nitro-pyridine-3-carboxylate (11.51 g, 97%) as a tan solid. ¹H NMR (400 MHz, DMSO-d₆) δ 12.44 (br s, 2H), 4.31 (q, J=7.0 Hz, 2H), 2.43 (s, 3H), 1.30 (t, J=7.1 Hz, 3H). ESI-MS m/z calc. 242.05, found 243.2 (M+1)⁺.

### Step 2: ethyl 4,6-dichloro-2-methyl-5-nitro-pyridine-3-carboxylate

Ethyl 4,6-dichloro-2-methyl-5-nitro-pyridine-3-carboxylate was prepared from ethyl 4,6-dihydroxy-2-methyl-5-nitro-pyridine-3-carboxylate using a procedure analogous to that found in Preparation 1, step 2. ¹H NMR (400 MHz, CDCl₃) δ 4.49 (q, J=7.1 Hz, 2H), 2.63 (s, 3H), 1.43 (t, J=7.1 Hz, 3H). ESI-MS m/z calc. 277.99, found 279.0 (M+1)⁺.

### Step 3: ethyl 5-amino-4,6-dichloro-2-methyl-pyridine-3-carboxylate

Ethyl 5-amino-4,6-dichloro-2-methyl-pyridine-3-carboxylate was prepared from ethyl 4,6-dichloro-2-methyl-5-nitro-pyridine-3-carboxylate using a procedure analogous to that found in

Intermediate A-10, step 2. ¹H NMR (400 MHz, DMSO-d₆) δ 5.89 (s, 2H), 4.37 (q, J=7.1 Hz, 2H), 2.26 (s, 3H), 1.31 (t, J=7.1 Hz, 3H). ESI-MS m/z calc. 248.01, found 249.2 (M+1)⁺.

### Step 4: ethyl 4,6-dichloro-5-(dimethylamino)-2-methyl-pyridine-3-carboxylate

To a solution of ethyl 5-amino-4,6-dichloro-2-methyl-pyridine-3-carboxylate (200 mg, 0.794 mmol) and sodium cyanoborohydride (300 mg, 4.77 mmol) in THF (4 mL) at 0° C. was added a solution of formaldehyde in water (0.30 mL of 37% w/w, 4.0 mmol) followed by the dropwise addition of sulfuric acid (0.25 mL, 4.7 mmol) over 5 min. The mixture was allowed to warm to room temperature and stirred for 2.75 h. A second portion of formaldehyde in w ater (0.30 mL of 37% w/w, 4.0 mmol) and sodium cyanoborohydride (150 mg, 2.39 mmol) were added to the mixture and it was stirred at room temperature for 1 h. A third addition of formaldehyde in water (328 mg, 0.30 mL of 37% w/w, 4.0 mmol) and sodium cyanoborohydride (150 mg, 2.39 mmol) was done and the mixture was stirred for 2 h at room temperature. The mixture was partitioned between water (50 mL) and ethyl acetate (30 mL) and the aqueous layer was extracted with additional ethyl acetate (2×30 mL). The combined organic layers were washed with brine (40 mL), dried over sodium sulfate, filtered and concentrated under reduced pressure. Purification by silica gel chromatography (24 g silica, 0-5% ethyl acetate/heptanes) afforded ethyl 4,6-dichloro-5-(dimethylamino)-2-methyl-pyridine-3-carboxylate (178 mg, 81%) as a light yellow oil. ¹H NMR (400 MHz, CDCl₃) δ 4.45 (q, J=7.2 Hz, 2H), 2.87 (s, 6H), 2.47 (s, 3H), 1.42 (t, J=7.1 Hz, 3H). ESI-MS m/z calc. 276.04, found 277.2 (M+1)⁺.

### Step 5: ethyl 4-benzyloxy-6-chloro-5-(dimethylamino)-2-methyl-pyridine-3-carboxylate

Ethyl 4-benzyloxy-6-chloro-5-(dimethylamino)-2-methyl-pyridine-3-carboxylate was prepared from ethyl 4,6-dichloro-5-(dimethylamino)-2-methyl-pyridine-3-carboxylate and benzyl alcohol using a procedure analogous to that found in Preparation 1, step 3. ¹H NMR (400 MHz, CDCl₃) δ 7.42-7.33 (m, 5H), 5.14 (s, 2H), 4.29 (q, J=7.1 Hz, 2H), 2.85 (s, 6H), 2.45 (s, 3H), 1.27 (t, J=7.1 Hz, 3H). ESI-MS m/z calc. 348.12, found 349.1 (M+1)⁺.

### Intermediate A-12

### 4-benzyloxy-2-chloro-6-vinyl-pyridine

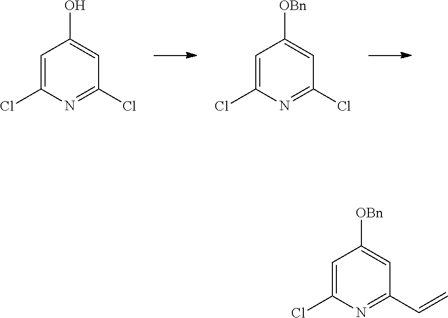 [SMILES: Oc1cc(Cl)nc(Cl)c1 ; Clc1cc(OCc2ccccc2)cc(Cl)n1 ; C=Cc1cc(OCc2ccccc2)cc(Cl)n1]

### Step 1: 4-benzyloxy-2,6-dichloro-pyridine

2,6-Dichloropyridin-4-ol (4.65 g, 28.36 mmol) was dissolved in acetonitrile (55 mL) then treated with cesium carbonate (13.9 g, 42.7 mmol) and benzyl bromide (8.4 mL, 71 mmol). The resulting mixture was stirred at 40° C. for 1 h, then filtered through Celite® and concentrated in vacuo. Purification by silica gel chromatography (0-35% ethyl acetate/heptane) provided 4-benzyloxy-2,6-dichloro-pyridine (6.91 g, 85%) as a white solid. ESI-MS m/z calc. 253.01, found 254.0 (M+1)⁺. ¹H NMR (400 MHz, CDCl₃) δ 7.48-7.33 (m, 5H), 6.86 (s, 2H), 5.11 (s, 2H).

### Step 2: 4-benzyloxy-2-chloro-6-vinyl-pyridine

To a solution of 4-benzyloxy-2,6-dichloro-pyridine (4.6 g, 18 mmol) in 1,4-dioxane (100 mL) and water (20 mL) was added potassium vinyltrifluoroborate (2.5 g, 19 mmol) and sodium carbonate (5.7 g, 54 mmol). The solution was bubbled with nitrogen then Pd(PPh₃)₄ (3.0 g, 2.6 mmol) added and the mixture bubbled with nitrogen for 15 min. The mixture was stirred at reflux for 18 h, then cooled and partitioned between ethyl acetate (200 mL) and water (300 mL). The aqueous phase was extracted with additional ethyl acetate (2×100 mL). The combined organic layers were washed with brine, dried over sodium sulfate, filtered and concentrated under reduced pressure. Purification by silica gel chromatography (0-10% of ethyl acetate/heptanes) provided 4-benzyloxy-2-chloro-6-vinyl-pyridine (2.55 g, 57%) as clear oil. ¹H NMR (400 MHz, CDCl₃) δ 7.49-7.35 (m, 5H), 6.93-6.79 (m, 2H), 6.69 (dd, J=17.4, 10.8 Hz, 1H), 6.25 (dd, J=17.4, 0.7 Hz, 1H), 5.53 (d, J=10.5 Hz, 1H), 5.13 (s, 2H). ESI-MS m/z calc. 245.06, found 246.0 (M+1)⁺.

After Suzuki coupling of 4-benzyloxy-2-chloro-6-vinyl-pyridine with Intermediate-B, the alkene may be reduced using standard hydrogenation conditions to provide the corresponding ethyl analog.

### Intermediate A-13

### (4-benzyloxy-2-chloro-6-methyl-3-pyridyl)methanol

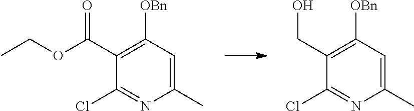 [SMILES: CCOC(=O)c1c(OCc2ccccc2)cc(C)nc1Cl ; Cc1cc(OCc2ccccc2)c(CO)c(Cl)n1]

Ethyl 4-benzyloxy-2-chloro-6-methyl-pyridine-3-carboxylate (Intermediate A-40, 150 mg, 0.491 mmol) was dissolved in THF (1.5 mL). The resulting solution was cooled to 0° C., then LAH (490 L of 1.0 M in THF, 0.49 mmol) in THF was added and the resulting solution was stirred for 4 h at 0° C. and was let to warm up to room temperature. The resulting solution was diluted with ethyl acetate (20 mL), washed with saturated solution of potassium sodium tartrate (Rochelle salt) and brine. Organic layer was dried over magnesium sulfate and concentrated under reduced pressure. The residue was purified reverse phase chromatography (C18, 10-99% acetonitrile/5 mM HCl) to provide (4-benzyloxy-2-chloro-6-methyl-3-pyridyl)methanol (126.3 mg, 98%), as a white solid. ESI-MS m/z calc. 263.07, found 164.12 (M+1)⁺. ¹H NMR (400 MHz, DMSO-d₆) δ 7.49 (d, J=7.0 Hz, 2H), 7.44-7.39 (m, 2H), 7.38-7.33 (m, 1H), 7.09 (s, 1H), 5.24 (s, 2H), 4.54 (s, 2H), 2.39 (s, 3H).

### Intermediate A-14

### 4-benzyloxy-2-chloro-3-(ethoxymethyl)-6-methyl-pyridine

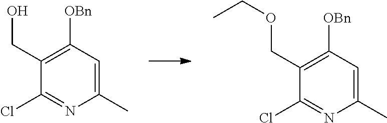 [SMILES: Cc1cc(OCc2ccccc2)c(CO)c(Cl)n1 ; CCOCc1c(OCc2ccccc2)cc(C)nc1Cl]

A stirring solution of (4-benzyloxy-2-chloro-6-methyl-3-pyridyl)methanol (188 mg, 0.713 mmol) in THF (3 mL) at 0° C. was treated with sodium hydride (53.6 mg, 60% dispersion in mineral oil, 1.34 mmol) then stirred at room temperature for 20 min. The reaction mixture was then cooled to 0° C. and iodoethane (172 μL, 2.15 mmol) was added dropwise. The mixture was allowed to warm up to room temperature and stirred at room temperature for 20 h. The mixture was cooled to 0° C., quenched carefully with ice water then partitioned between ethyl acetate and water. The layers were separated and the aqueous layer extracted with additional ethyl acetate (2×). The combined organic layers were dried over sodium sulfate, filtered and concentrated. Purification by silica gel chromatography (0-30% ethyl acetate/hexanes) provided 4-benzyloxy-2-chloro-3-(ethoxymethyl)-6-methyl-pyridine (140.9 mg, 68%). ¹H NMR (400 MHz, DMSO-d₆) δ 7.50-7.31 (m, 5H), 7.12 (s, 1H), 5.26 (s, 2H), 4.50 (s, 2H), 3.45 (q, J=7.0 Hz, 2H), 2.40 (s, 3H), 1.08 (t, J=7.0 Hz, 3H). ESI-MS m/z calc. 291.10, found 292.2 (M+1)⁺.

### Intermediate A-15

### Ethyl 4-benzyloxy-2-chloro-5,6-dimethyl-pyridine-3-carboxylate

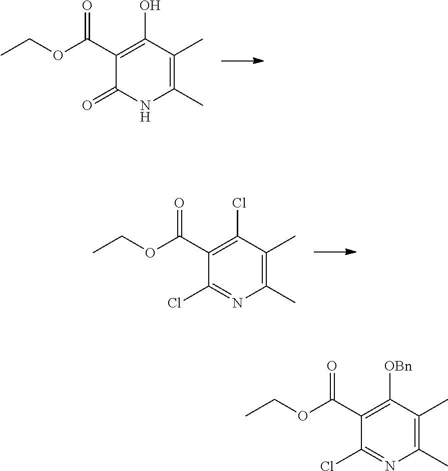 [SMILES: CCOC(=O)c1c(O)c(C)c(C)[nH]c1=O ; CCOC(=O)c1c(Cl)nc(C)c(C)c1Cl ; CCOC(=O)c1c(Cl)nc(C)c(C)c1OCc1ccccc1]

### Step 1: ethyl 2,4-dichloro-5,6-dimethyl-pyridine-3-carboxylate

Ethyl 4-hydroxy-5,6-dimethyl-2-oxo-1H-pyridine-3-carboxylate (500 mg, 2.37 mmol) was dissolved in POCl₃ (1.5 mL, 16 mmol) and the solution was heated at 105° C. for 14 h. After cooling to room temperature, the mixture was poured onto ice and stirred for 30 min. The residue was dissolved in ethyl acetate and was carefully washed with saturated sodium bicarbonate solution (3×5 mL), filtered, and concentrated in vacuo. Purification by silica gel chromatography (0-30% ethyl acetate/hexanes) provided ethyl 2,4-dichloro-5,6-dimethyl-pyridine-3-carboxylate (494 mg, 83%) as a white solid. ESI-MS m/z calc. 247.02, found 248.1 (M+1)⁺.

### Step 2: ethyl 4-benzyloxy-2-chloro-5,6-dimethyl-pyridine-3-carboxylate

Ethyl 4-benzyloxy-2-chloro-5,6-dimethyl-pyridine-3-carboxylate was prepared from ethyl 2,4-dichloro-5,6-dimethyl-pyridine-3-carboxylate and benzyl alcohol using a procedure analogous to that found in Preparation 1, step 3. ESI-MS m/z calc. 319.1, found 320.0 (M+1)⁺.

### Intermediate A-16

### ethyl 4-benzyloxy-6-chloro-2,5-dimethyl-pyridine-3-carboxylate

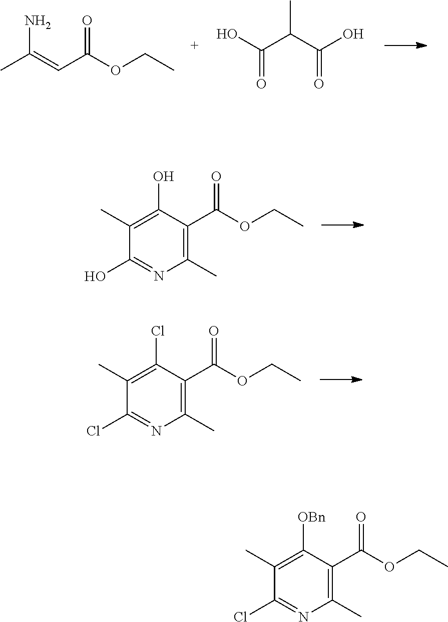

### Step 1: ethyl 4,6-dihydroxy-2,5-dimethyl-pyridine-3-carboxylate

A mixture of ethyl (Z)-3-aminobut-2-enoate (32.7 g, 32.0 mL, 253 mmol) and 2-methylpropanedioic acid (30.0 g, 254 mmol) in acetic anhydride (260 g, 240 mL, 2.54 mol) was heated at 100° C. for 3 h then allowed to cool. The resulting solid was collected by filtration, washed with ethyl acetate (100 mL) and dried provided ethyl 4,6-dihydroxy-2,5-dimethyl-pyridine-3-carboxylate (20.15 g, 37%). ¹H NMR (400 MHz, DMSO-d₆) δ 11.71 (s, 1H), 11.45 (s. 1H), 4.28 (q, J=7.2 Hz, 2H), 2.43 (s, 3H), 1.76 (s, 3H), 1.28 (t, J=7.1 Hz, 3H). EST-MS m/z calc. 211.09, found 212.02 (M+1)⁺.

### Step 2: ethyl 4,6-dichloro-2,5-dimethyl-pyridine-3-carboxylate

A mixture of ethyl 4,6-dihydroxy-2,5-dimethyl-pyridine-3-carboxylate (20.15 g, 95.15 mmol) and tetramethylammonium chloride (11.5 g, 105 mmol) in POCl₃ (146 g, 89 mL. 955 mmol) was heated at reflux for 4 h then left to cool overnight. The mixture was carefully added dropwise to cold water (200 mL). additional water (200 mL) was added and the aqueous phase was extracted with ethyl acetate (2×200 mL). The combined extracts were washed with water (100 mL) and brine (100 mL), dried over sodium sulfate and concentrated to provide ethyl 4,6-dichloro-2,5-dimethyl-pyridine-3-carboxylate (24.13 g, 100%). ¹H NMR (400 MHz, CDCl₃) δ 4.43 (q, J=7.0 Hz, 2H), 2.48 (s, 3H), 2.44 (s, 3H), 1.40 (q. J=7.2 Hz, 3H). ESI-MS m/z calc. 247.02, found 247.91 (M+1)⁺.

### Step 3: ethyl 4-benzyloxy-6-chloro-2,5-dimethyl-pyridine-3-carboxylate

Ethyl 4-benzyloxy-6-chloro-2,5-dimethyl-pyridine-3-carboxylate was prepared from ethyl 4,6-dichloro-2,5-dimethyl-pyridine-3-carboxylate and benzyl alcohol using a procedure analogous to that found in Preparation 1, step 3. ¹H NMR (400 MHz, CDCl₃) δ 7.42-7.40 (m, 5H), 5.01 (s, 2H), 4.37 (q, J=7.2 Hz, 2H), 2.52 (s, 3H), 2.29 (s, 3H), 1.34 (t, J=7.1 Hz, 3H). ESI-MS m/z calc. 319.10, found 320.01 (M+1)⁺.

### Intermediate A-17

### [6-chloro-4-[(4-methoxyphenyl)methoxy]-2-methyl-3-pyridyl]methanol

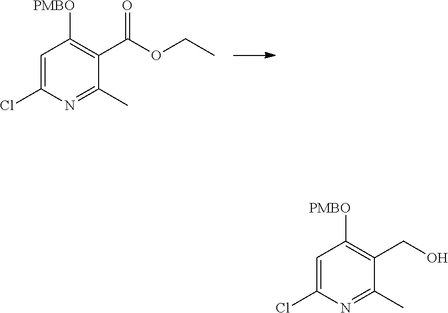

To a stirring solution of ethyl 6-chloro-4-[(4-methoxyphenyl)methoxy]-2-methyl-pyridine-3-carboxylate (Intermediate A-39, 375 mg, 1.12 mmol) in THF (5 mL) at 0° C. was added LAH (0.8 mL of 2.0 M in THF, 1.6 mmol) portionwise. The mixture was stirred at 0° C. for 30 min then stirred at room temperature for 4 h. The mixture was cooled to 0° C. and quenched with saturated sodium sulfate solution, filtered through Celite® and extracted with DCM (2×10 mL). The organic phase was dried over sodium sulfate and concentrated under reduced pressure to afford [6-chloro-4-[(4-methoxyphenyl)methoxy]-2-methyl-3-pyridyl]methanol (350 mg, 107%). ESI-MS m/z calc. 293.08, found 294.0 (M+1)⁺.

### Intermediate A-18

### 1-[6-chloro-4-[(4-methoxyphenyl)methoxy]-2-methyl-3-pyridyl]ethanone

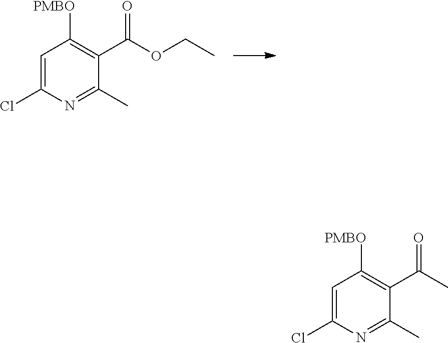

To a solution of ethyl 6-chloro-4-[(4-methoxyphenyl)methoxy]-2-methyl-pyridine-3-carboxylate (100 mg, 0.294 mmol) in THF (2 mL) at −5° C. was slowly added methylmagnesium bromide solution (0.25 mL of 3.0 M in diethyl ether, 0.75 mmol). The reaction mixture was stirred at 0° C. for 3 h. An additional amount of methylmagnesium bromide solution (0.2 mL of 3.0 M in diethyl ether, 0.6 mmol) was added and the reaction mixture stirred at 0° C. for 2 h. The mixture was quenched with aqueous ammonium chloride solution and extracted with ethyl acetate. The organic layer was dried over sodium sulfate, filtered and concentrated. Purification by reverse phase chromatography (C18, 10-70% acetonitrile/water, each with 0.1% formic acid) provided 1-[6-chloro-4-[(4-methoxyphenyl)methoxy]-2-methyl-3-pyridyl]ethanone (20 mg, 21%). ¹H NMR (400 MHz, CDCl₃) δ 7.27 (dd, J=6.6, 2.1 Hz, 2H), 6.92 (dd, J=6.6, 2.1 Hz, 2H), 6.81 (s, 1H), 5.05 (s, 2H), 3.82 (s, 3H), 2.44 (s, 3H), 2.41 (s, 3H).

### Intermediate A-19

### benzyl 4-benzyloxy-2-chloro-5,6-dimethyl-pyridine-3-carboxylate

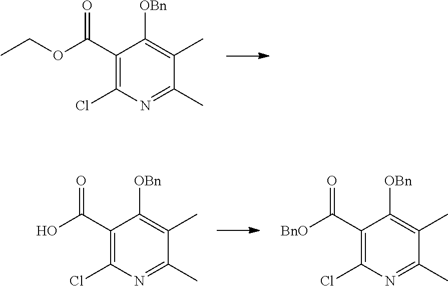 [SMILES: CCOC(=O)c1c(Cl)nc(C)c(C)c1OCc1ccccc1 ; Cc1nc(Cl)c(C(=O)O)c(OCc2ccccc2)c1C ; Cc1nc(Cl)c(C(=O)OCc2ccccc2)c(OCc2ccccc2)c1C]

### Step 1: 4-benzyloxy-2-chloro-5,6-dimethyl-pyridine-3-carboxylic acid

A solution of ethyl 4-benzyloxy-2-chloro-5,6-dimethyl-pyridine-3-carboxylate (Intermediate A-15, 400 mg, 1.23 mmol) in methanol (6 mL) and THF (4 mL) was treated with aqueous NaOH (7 mL of 1 M, 7 mmol) and stirred overnight at 65° C. The mixture was quenched with aqueous 1 N HCl, diluted with ethyl acetate, and washed with saturated aqueous ammonium chloride and brine. The organic layer was dried over magnesium sulfate, filtered and concentrated under reduced pressure. The solid was triturated in dichloromethane/hexanes (2:1) then filtered to provide 4-benzyloxy-2-chloro-5,6-dimethyl-pyridine-3-carboxylic acid (318 mg, 89%). ¹H NMR (400 MHz, DMSO-d₆) δ 13.96 (br s, 1H), 7.49-7.34 (m, 5H), 5.00 (s, 2H), 2.43 (s, 3H), 2.16 (s, 3H). ESI-MS m/z calc. 291.07, found 292.2 (M+1)⁺.

### Step 2: benzyl 4-benzyloxy-2-chloro-5,6-dimethyl-pyridine-3-carboxylate

A solution of 4-benzyloxy-2-chloro-5,6-dimethyl-pyridine-3-carboxylic acid (62 mg, 0.20 mmol) in DCM (2 mL) was treated with benzyl alcohol (50 μL, 0.48 mmol), DIPEA (40 μL, 0.23 mmol) and DMAP (21 mg, 0.17 mmol). PyBOP (105 mg, 0.202 mmol) was added and the resulting mixture was stirred for 3 h. The mixture was quenched with 1 N HCl solution, diluted with ethyl acetate, washed with saturated solution of ammonium chloride and brine. The organic layer was dried over magnesium sulfate, filtered and concentrated under reduced pressure. Purification by silica gel chromatography (0-40% ethyl acetate/hexanes over 20 min) afforded benzyl 4-benzyloxy-2-chloro-5,6-dimethyl-pyridine-3-carboxylate (64.1 mg, 77%) as a clear thick oil which crystallized upon standing. ¹H NMR (400 MHz, DMSO-d₆) δ 7.50-7.28 (m, 10H), 5.36 (s, 2H), 4.91 (s, 2H), 2.44 (s, 3H), 2.15 (s, 3H). ESI-MS m/z calc. 381.11, found 382.36 (M+1)⁺.

### Intermediate A-20

### isopropyl 4-benzyloxy-2-chloro-5,6-dimethyl-pyridine-3-carboxylate

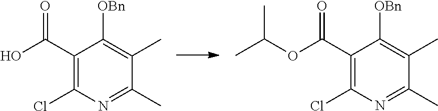 [SMILES: Cc1nc(Cl)c(C(=O)O)c(OCc2ccccc2)c1C ; Cc1nc(Cl)c(C(=O)OC(C)C)c(OCc2ccccc2)c1C]

Isopropyl 4-benzyloxy-2-chloro-5,6-dimethyl-pyridine-3-carboxylate was prepared from 4-benzyloxy-2-chloro-5,6-dimethyl-pyridine-3-carboxylic acid and 2-propanol using a procedure analogous to that found in Intermediate A-19. ¹H NMR (400 MHz, DMSO-d₆) δ 7.47-7.33 (m, 5H), 5.16 (hept, J=6.2 Hz, 1H), 4.99 (s, 2H), 2.44 (s, 3H), 2.16 (s, 3H), 1.26 (d, J=6.3 Hz, 6H). ESI-MS m/z calc. 333.11, found 334.645 (M+1)⁺.

### Intermediate A-21

### ethyl 4-benzyloxy-2-chloro-5-cyano-6-methyl-pyridine-3-carboxylate

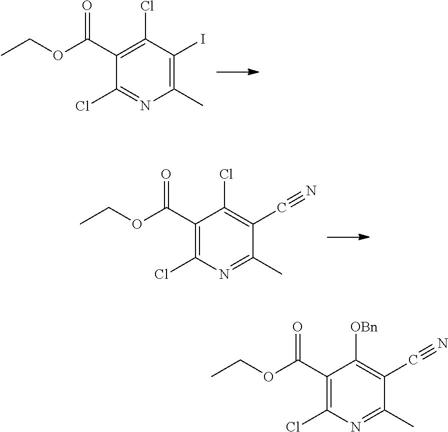 [SMILES: CCOC(=O)c1c(Cl)nc(C)c(I)c1Cl ; CCOC(=O)c1c(Cl)nc(C)c(C#N)c1Cl ; CCOC(=O)c1c(Cl)nc(C)c(C#N)c1OCc1ccccc1]

### Step 1: ethyl 2,4-dichloro-5-cyano-6-methyl-pyridine-3-carboxylate

A mixture of ethyl 2,4-dichloro-5-iodo-6-methyl-pyridine-3-carboxylate (Preparation 1, 100 mg, 0.280 mmol) and CuCN (28 mg, 0.31 mmol) in NMP (1 mL) was degassed under an atmosphere of nitrogen then heated in a scaled vial at 100° C. for 16 h. The mixture was filtered and washed with ethyl acetate. The filtrate was washed with brine (3×), dried over magnesium sulfate, filtered and concentrated. Purification by silica gel chromatography (0-10% ethyl acetate/hexanes) provided ethyl 2,4-dichloro-5-cyano-6-methyl-pyridine-3-carboxylate (49 mg, 68%). ESI-MS m/z calc. 258.0, found 259.1 (M+1)⁺. ¹H NMR (400 MHz, DMSO-d₆) δ 4.46 (q, J=7.1 Hz, 2H), 2.71 (s, 3H), 1.34 (t, J=7.1 Hz, 3H). ¹³C NMR (101 MHz, DMSO-d₆) δ 165.17, 162.35, 149.23, 144.89, 127.34, 114.01, 110.57, 63.79, 24.26, 14.24.

### Step 2: ethyl 4-benzyloxy-2-chloro-5-cyano-6-methyl-pyridine-3-carboxylate

Ethyl 4-benzyloxy-2-chloro-5-cyano-6-methyl-pyridine-3-carboxylate was prepared from ethyl 2,4-dichloro-5-cyano-6-methyl-pyridine-3-carboxylate and benzyl alcohol using a procedure analogous to that found in Preparation 1, step 3. ESI-MS m/z calc. 330.08, found 331.2 (M+1)⁺. ¹H NMR (400 MHz, DMSO-d₆) δ 7.51-7.37 (m, 5H), 5.47 (s, 2H), 4.33 (d, J=7.2 Hz, 2H), 3.29 (s, 3H), 1.22 (t, J=7.1 Hz, 3H).

### Intermediate A-22

### ethyl 4-benzyloxy-2-chloro-5-ethynyl-6-methyl-pyridine-3-carboxylate

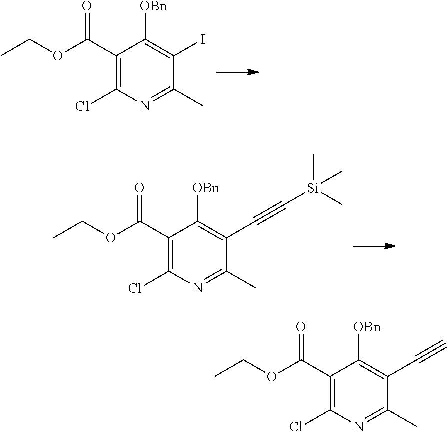

### Step 1: ethyl 4-benzyloxy-2-chloro-6-methyl-5-(2-trimethylsilylethynyl)pyridine-3-carboxylate

A mixture of ethyl 4-benzyloxy-2-chloro-5-iodo-6-methyl-pyridine-3-carboxylate (Preparation 1, 100 mg, 0.232 mmol), ethynyl(trimethyl)silane (150 μL, 1.06 mmol), Na₂PdCl₄ (15.2 mg, 0.0517 mmol), CuI (10 mg, 0.053 mmol) and P(^{t}Bu)₃ (19.2 mg, 0.095 mmol) in diisopropylamine (300 μL) was degassed for 1 min then stirred in a sealed tube at 80° C. for 1 h under nitrogen. The mixture was cooled to room temperature, diluted with ethyl acetate (20 mL) and washed with saturated aqueous ammonium chloride and brine. The organic layer was dried over magnesium sulfate, filtered, and concentrated under reduced pressure. Purification by silica gel chromatography (0-40% ethyl acetate/hexanes over 15 min) provided ethyl 4-benzyloxy-2-chloro-6-methyl-5-(2-trimethylsilylethynyl)pyridine-3-carboxylate. ESI-MS m/z calc. 401.12, found 402.5 (M+1)⁺.

### Step 2: ethyl 4-benzyloxy-2-chloro-5-ethynyl-6-methyl-pyridine-3-carboxylate

Ethyl 4-benzyloxy-2-chloro-6-methyl-5-(2-trimethylsilylethynyl)pyridine-3-carboxylate from step 1 was dissolved in methanol (2 mL) followed by the addition of potassium carbonate (33.5 mg, 0.242 mmol). The mixture was stirred at room temperature for 1 h, then diluted with ethyl acetate and washed with saturated aqueous ammonium chloride and brine. The organic layer was dried over magnesium sulfate, filtered, and concentrated. Purification by silica gel chromatography (0-30% ethyl acetate/heptanes over 15 min) provided ethyl 4-benzyloxy-2-chloro-5-ethynyl-6-methyl-pyridine-3-carboxylate (60 mg, 79%). ¹H NMR (400 MHz, DMSO-d₆) δ 7.45-7.33 (m, 5H), 5.38 (s, 2H), 5.10 (s, 1H), 4.27 (q, J=7.1 Hz, 2H), 2.59 (s, 3H), 1.20 (t, J=7.1 Hz, 3H). ESI-MS m/z calc. 329.08, found 330.3 (M+1)⁺.

### Intermediate A-23

### 4-(benzyloxy)-6-chloro-2-methyl-3-(methylthio)pyridine

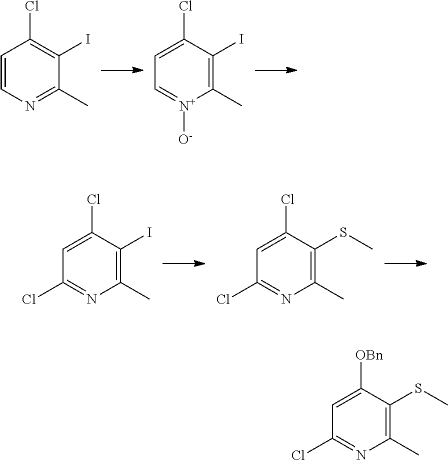

### Step 1: 4-chloro-3-iodo-2-methylpyridine 1-oxide

A solution of mCPBA (23.4 g, 70-75% w/w in water, 101.7 mmol) was added portionwise to a solution of 4-chloro-3-iodo-2-methylpyridine (17.2 g, 67.9 mmol) in DCM (250 mL) at 0° C. The reaction mixture was warmed to room temperature and stirred overnight. The mixture was diluted with DCM (250 mL) and washed with water (250 mL). The aqueous layer was separated and extracted with DCM (200 mL). The combined organic extracts were dried over sodium sulfate, filtered and concentrated in vacuo. The residue was triturated in methanol (2×30 mL), filtered and dried to provided 4-chloro-3-iodo-2-methylpyridine 1-oxide (15 g, 82%) as a white solid. ¹H NMR (400 MHz, CDCl₃) δ 8.17 (d, J=7.3 Hz, 1H), 7.23 (d, J=6.9 Hz, 1H), 2.90 (s, 3H). ESI-MS m/z calc. 268.91, found 269.78 (M+1)⁺.

### Step 2: 4,6-dichloro-3-iodo-2-methylpyridine

A solution of 4-chloro-3-iodo-2-methylpyridine 1-oxide (1.75 g, 6.45 mmol) in POCl₃ (16.5 g, 10.0 mL, 107 mmol) was heated at 85° C. for 5 h. The reaction mixture was cooled and concentrated in vacuo. The residue was partitioned between ethyl acetate (50 mL) and water (20 mL). The aqueous layer was separated and extracted with additional ethyl acetate (2×20 mL). The combined organic extracts were dried over sodium sulfate, filtered and concentrated in vacuo to provide 4,6-dichloro-3-iodo-2-methylpyridine (2.5 g, 70%). ESI-MS m/z calc. 286.88, found 287.73 (M+1)⁺.

### Step 3: 4,6-dichloro-2-methyl-3-(methylthio)pyridine

A mixture of 4,6-dichloro-3-iodo-2-methylpyridine (212 mg, 0.710 mmol), sodium methanethiolate (50 mg, 0.70 mmol), Pd₂dba₃ (16 mg, 0.02 mmol), Xantphos (20 mg, 0.04 mmol) and DIPEA (185 mg, 250 μL, 1.42 mmol) in a mixture of 1,4-dioxane (2 mL) and water (0.2 mL) was stirred under microwave irradiation at 100° C. for 1 h. The mixture was partitioned between ethyl acetate and water. The aqueous phase was separated and extracted with ethyl acetate. The combined organic extracts were washed with brine, dried over magnesium sulfate, filtered and concentrated in vacuo. Purification by silica gel chromatography (0-20% ethyl acetate/heptane) provided 4,6-dichloro-2-methyl-3-(methylthio)pyridine (186 mg, 94%). ¹H NMR (400 MHz, CDCl₃) δ 7.30 (s, 1H), 2.78 (s, 3H), 2.34 (s, 3H). ESI-MS m/z calc. 206.97, found 207.94 (M+1)⁺.

### Step 4: 4-(benzyloxy)-6-chloro-2-methyl-3-(methylthio)pyridine

4-(benzyloxy)-6-chloro-2-methyl-3-(methylthio)pyridine was prepared from_4,6-dichloro-2-methyl-3-(methylthio)pyridine and benzyl alcohol using a procedure analogous to that found in Preparation 1, step 3 and NMP as the solvent. ¹H NMR (400 MHz, CDCl₃) δ 7.47-7.35 (m, 5H), 6.76 (s, 1H), 5.18 (s, 2H), 2.69 (s, 3H), 2.30 (s, 3H). ESI-MS m/z calc. 279.05, found 280.4 (M+1)⁺.

### Intermediate A-24

### (4-benzyloxy-6-chloro-2-methyl-3-pyridyl)-imino-methyl-oxo-λ6-sulfane

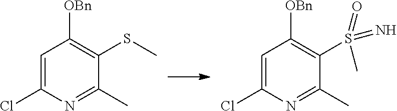 [SMILES: CSc1c(OCc2ccccc2)cc(Cl)nc1C ; Cc1nc(Cl)cc(OCc2ccccc2)c1S(C)(=N)=O]

4-(Benzyloxy)-6-chloro-2-methyl-3-(methylthio)pyridine (2.0 g, 7.2 mmol) was dissolved in DCM (20 mL) and methanol (20 mL) and the solution cooled to 0° C. Ammonium carbamate (837 mg, 10.7 mmol) and (diacetoxyiodo)benzene (4.61 g, 14.3 mmol) were added and the mixture was allowed to warm to room temperature and stirred for 2 h. Additional (diacetoxyiodo)benzene (1.15 g, 3.57 mmol) and ammonium carbamate (223 mg, 2.86 mmol) were added and the resulting mixture was left to stir at room temperature for 1.5 h. The reaction mixture was concentrated in vacuo and purified by silica gel column chromatography (0-100% ethyl acetate/heptane) to provide (4-benzyloxy-6-chloro-2-methyl-3-pyridyl)-imino-methyl-oxo-λ6-sulfane (1.314 g, 59%) as a white solid. ESI-MS m/z calc. 310.05, found 311.2 (M+1)⁺. ¹H NMR (500 MHz, DMSO-d₆) δ 7.56-7.54 (m, 2H), 7.45-7.40 (m, 2H), 7.39-7.35 (m, 2H), 5.39 (d, J=2.0 Hz, 2H), 4.59 (s, 1H), 3.17 (d, J=1.2 Hz, 3H), 2.74 (s, 3H).

The enantiomers were separated by chiral SFC using a ChiralPak IC (250×20 mm), 5 um, (Daicel Corp.) column at 40° C. using a Nexera UC Prep (Shimadzu). Separation was achieved using an isocratic method where the mobile phase was 20% methanol (20 mM NH₃), 80% CO₂ at a flow rate of 100 mL/min. Detection wavelength was 215 nm. Retention time of Peak 1 was 4.380 min and Peak 2 was 5.468 min.

Peak 1: (4-benzyloxy-6-chloro-2-methyl-3-pyridyl)-imino-methyl-oxo-λ6-sulfane (561 mg, 51%), ESI-MS m/z calc. 310.05, found 311.1 (M+1); Retention time: 2.19 min. 98.1% ee.

Peak 2: (4-benzyloxy-6-chloro-2-methyl-3-pyridyl)-imino-methyl-oxo-λ6-sulfane (523 mg, 47%), ESI-MS m/z calc. 310.05, found 311.1 (M+1)⁺; Retention time: 2.19 min. 91.1% ee.

### Intermediate A-25

### ethyl 4,5-dibenzyloxy-2-chloro-6-methyl-pyridine-3-carboxylate

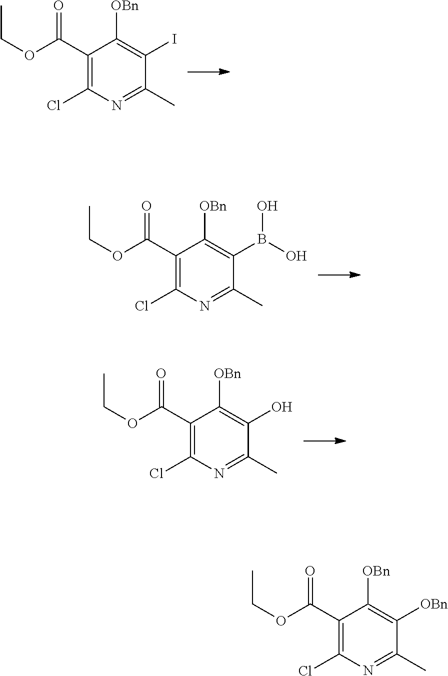 [SMILES: CCOC(=O)c1c(Cl)nc(C)c(I)c1OCc1ccccc1 ; CCOC(=O)c1c(Cl)nc(C)c(B(O)O)c1OCc1ccccc1 ; CCOC(=O)c1c(Cl)nc(C)c(O)c1OCc1ccccc1 ; CCOC(=O)c1c(Cl)nc(C)c(OCc2ccccc2)c1OCc1ccccc1]

### Step 1: (4-benzyloxy-6-chloro-5-ethoxycarbonyl-2-methyl-3-pyridyl)boronic acid

Ethyl 4-benzyloxy-2-chloro-5-iodo-6-methyl-pyridine-3-carboxylate (Preparation 1, 236 mg, 0.547 mmol) was dissolved in Et₂O (5 mL). The resulting mixture was cooled to −78° C. Then n-BuLi (250 μL of 2.5 M, 0.63 mmol) was added dropwise over 5 min and the resulting mixture was stirred at −78° C. for 15 min. (MeO)₃B (250 μL, 2.20 mmol) was added dropwise and the resulting mixture was allowed to stir for 2 h at −78° C. After 2 h the reaction was quenched with saturated aqueous solution of ammonium chloride, diluted with ethyl acetate and washed with brine. Organic layer was dried over magnesium sulfate, filtered and concentrated under reduced pressure to yield a mixture of (4-benzyloxy-6-chloro-5-ethoxycarbonyl-2-methyl-3-pyridyl)boronic acid, ESI-MS m/z calc. 349.09, found 350.2 (M+1)⁺; and ethyl 4-benzyloxy-2-chloro-6-methyl-pyridine-3-carboxylate, ESI-MS m/z calc. 305.08, found 306.2 (M+1)⁺.

### Step 2: ethyl 4-benzyloxy-2-chloro-5-hydroxy-6-methyl-pyridine-3-carboxylate

(4-Benzyloxy-6-chloro-5-ethoxycarbonyl-2-methyl-3-pyridyl)boronic acid from step 1 was dissolved in Et₂O (2 mL) and THF (2 mL), cooled to 0° C. and then H₂O₂ (200 μL of 35% w/w, 2.34 mmol) was added dropwise followed by aqueous NaOH (150 μL of 1 M, 0.15 mmol). The mixture was stirred for 4 h at room temperature, then cooled to 0° C. and quenched with saturated aqueous sodium thiosulfate (2 mL). The mixture was warmed up to room temperature, diluted with ethyl acetate and washed with brine. Organic layer was dried over magnesium sulfate, filtered and concentrated under reduced pressure. Purification by silica gel chromatography (0-50% ethyl acetate/hexanes over 15 min) provided a 3:2 mixture of ethyl 4-benzyloxy-2-chloro-5-hydroxy-6-methyl-pyridine-3-carboxylate, ESI-MS m/z calc. 321.08, found 322.256 (M+1) and ethyl 4-benzyloxy-2-chloro-6-methyl-pyridine-3-carboxylate, ESI-MS m/z calc. 305.08, found 306.2 (M+1)⁺.

### Step 3: ethyl 4,5-dibenzyloxy-2-chloro-6-methyl-pyridine-3-carboxylate

The resulting mixture from step 2 was dissolved in DMF (2 mL) and cooled to 0° C. Benzyl bromide (30 μL, 0.25 mmol) and cesium carbonate (89 mg, 0.27 mmol) were added and the mixture stirred for 30 min. The mixture was diluted with ethyl acetate, washed with saturated aqueous ammonium chloride and brine. Organic layer was dried over magnesium sulfate, filtered and concentrated under reduced pressure. Purification by silica gel chromatography (0-30% ethyl acetate/hexanes over 15 min) provided ethyl 4,5-dibenzyloxy-2-chloro-6-methyl-pyridine-3-carboxylate (74.6 mg, 33%). ¹H NMR (400 MHz, DMSO-d₆) δ 7.50-7.29 (m, 10H), 5.24 (s, 2H), 5.02 (s, 2H), 4.27 (q, J=7.1 Hz, 2H), 2.35 (s, 3H), 1.20 (t, J=7.1 Hz, 3H). ESI-MS m/z calc. 411.12, found 412.4 (M+1)⁺.

### Intermediate A-26

### 2-(4-benzyloxy-6-chloro-2-methyl-3-pyridyl)acetamide

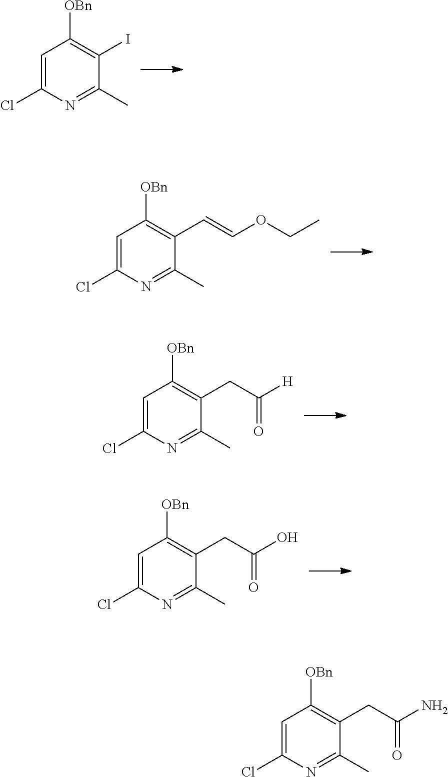 [SMILES: Cc1nc(Cl)cc(OCc2ccccc2)c1I ; CCO/C=C/c1c(OCc2ccccc2)cc(Cl)nc1C ; Cc1nc(Cl)cc(OCc2ccccc2)c1CC=O ; Cc1nc(Cl)cc(OCc2ccccc2)c1CC(=O)O ; Cc1nc(Cl)cc(OCc2ccccc2)c1CC(N)=O]

### Step 1: 4-benzyloxy-6-chloro-3-[(E)-2-ethoxyvinyl]-2-methyl-pyridine

A mixture of 4-benzyloxy-6-chloro-3-iodo-2-methyl-pyridine (314 mg, 0.87 mmol) (Preparation 2, step 2), 2-[(E)-2-ethoxyvinyl]-4,4,5,5-tetramethyl-1,3,2-dioxaborolane (173 mg, 0.87 mmol), PdCl₂(dtbpf) (66 mg, 0.1 mmol), potassium phosphate (550 mg, 2.6 mmol) in 1,4-dioxane (3 mL) and water (1 mL) was degassed for 5 min and heated in a sealed vial at 110° C. for 20 h under nitrogen. The mixture was cooled and diluted with ethyl acetate. The organic layer washed with a saturated aqueous solution of ammonium chloride and brine, dried over magnesium sulfate, filtered, and concentrated under reduced pressure. Purification by silica gel column chromatography (0-50% of ethyl acetate/hexanes) gave 4-benzyloxy-6-chloro-3-[(E)-2-ethoxyvinyl]-2-methyl-pyridine (188 mg, 71%). ESI-MS m/z calc. 303.10, found 304.3 (M+1)⁺. ¹H NMR (400 MHz, CDCl₃) δ 7.46-7.32 (m, 5H), 7.13 (d, J=12.7 Hz, 1H), 6.76 (s, 1H), 5.73 (d, J=12.7 Hz, 1H), 5.11 (s, 2H), 3.81 (q, J=7.0 Hz, 2H), 2.51 (s, 3H), 1.26 (t, J=7.0 Hz, 3H). ¹H NMR (400 MHz, CD₃OD) δ 7.54-7.32 (m, 5H), 7.16 (d, J=12.7 Hz, 1H), 7.02 (s, 1H), 5.70 (d, J=12.7 Hz, 1H), 5.20 (s, 2H), 3.80 (q, J=7.0 Hz, 2H), 2.44 (s, 3H), 1.21 (t, J=7.1 Hz, 3H).

### Step 2: 2-(4-benzyloxy-6-chloro-2-methyl-3-pyridyl)acetaldehyde

To a solution of 4-benzyloxy-6-chloro-3-[(E)-2-ethoxyvinyl]-2-methyl-pyridine in acetone (2 mL) from step 1 was added aqueous HCl (2 mL of 1 M, 2 mmol) and the mixture was stirred at 65° C. for 3 h. The mixture was diluted with ethyl acetate and washed with a saturated aqueous solution of ammonium chloride and brine. The organic layer was dried over magnesium sulfate, filtered, and then concentrated under reduced pressure to provide 2-(4-benzyloxy-6-chloro-2-methyl-3-pyridyl)acetaldehyde.

### Step 3: 2-(4-benzyloxy-6-chloro-2-methyl-3-pyridyl)acetic acid

A solution of 2-(4-benzyloxy-6-chloro-2-methyl-3-pyridyl)acetaldehyde from step 2 in DMF (2 mL) was treated with OXONE (545 mg, 0.89 mmol) and stirred at room temperature for 1 h. The mixture was diluted with ethyl acetate and washed with a saturated aqueous solution of ammonium chloride and brine. The organic layer was dried over anhydrous magnesium sulfate, filtered, and then concentrated under reduced pressure. Purification by silica gel column chromatography (0-70% ethyl acetate/heptanes) provided 2-(4-benzyloxy-6-chloro-2-methyl-3-pyridyl)acetic acid (125 mg, 49%). ESI-MS m/z calc. 291.07, found 292.3 (M+1)⁺. ¹H NMR (400 MHz, DMSO-d₆) δ 12.45 (br s, 1H), 7.46-7.30 (m, 5H), 7.09 (s, 1H), 5.24 (s, 2H), 3.60 (s, 2H), 2.36 (s, 3H).

### Step 4: 2-(4-benzyloxy-6-chloro-2-methyl-3-pyridyl)acetamide

A solution of 2-(4-benzyloxy-6-chloro-2-methyl-3-pyridyl)acetic acid (55 mg, 0.19 mmol) in DMF (550 μL) was treated with HATU (80 mg, 0.21 mmol), DIPEA (50 μL, 0.29 mmol) and NH₃ in methanol (150 μL of 7 M, 1.0 mmol) and stirred for 30 min. The mixture was diluted with ethyl acetate and washed with a saturated aqueous solution of ammonium chloride and brine. The organic layer was dried over magnesium sulfate, filtered, and concentrated under reduced pressure. Purification by silica gel column chromatography (0-100% ethyl acetate/hexanes) gave 2-(4-benzyloxy-6-chloro-2-methyl-3-pyridyl)acetamide (47 mg, 81%). ESI-MS m/z calc. 290.08, found 291.3 (M+1)⁺. ¹H NMR (400 MHz, DMSO-d₆) δ 7.50-7.28 (m, 6H), 7.05 (s, 1H), 6.94 (br s, 1H), 5.23 (s, 2H), 3.47 (s, 2H), 2.34 (s, 3H).

### Intermediate A-27

### diethyl 4-benzyloxy-2-chloro-6-methyl-pyridine-3,5-dicarboxylate

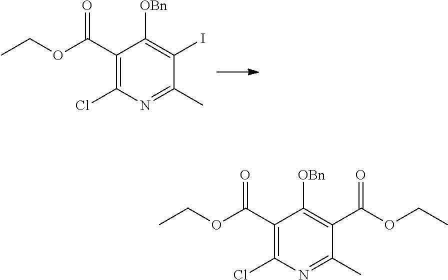 [SMILES: CCOC(=O)c1c(Cl)nc(C)c(I)c1OCc1ccccc1 ; CCOC(=O)c1c(C)nc(Cl)c(C(=O)OCC)c1OCc1ccccc1]

Ethyl 4-benzyloxy-2-chloro-5-iodo-6-methyl-pyridine-3-carboxylate (Preparation 1, 265 mg, 0.6139 mmol) was dissolved in Et₂O (6 mL). The resulting mixture was cooled to −78° C. and placed under a nitrogen atmosphere. n-BuLi (300 μL of 2.5 M, 0.75 mmol) was added dropwise over 5 min and the resulting mixture was stirred at −78° C. for 15 min. Ethyl carbonochloridate (200 μL, 2.09 mmol) was added dropwise and the mixture stirred for 60 min at −78° C. After 60 min the reaction was placed into a 0° C. bath and quenched with a saturated aqueous ammonium chloride (5 mL). The mixture was diluted with ethyl acetate and washed with brine. The organic layer was dried over magnesium sulfate, filtered and concentrated under reduced pressure. Purification by silica gel chromatography (0-40% ethyl acetate/hexanes over 20 min) provided diethyl 4-benzyloxy-2-chloro-6-methyl-pyridine-3,5-dicarboxylate (145 mg, 63%). ¹H NMR (400 MHz, DMSO-d₆) δ 7.46-7.31 (m, 5H), 5.10 (s, 2H), 4.34 (q, J=7.1 Hz, 2H) overlaps with 4.34 (q, J=7.1 Hz, 2H), 2.46 (s, 3H), 1.24 (t, J=7.1 Hz, 6H) (two overlapping triplets). ESI-MS m/z calc. 377.10, found 378.4 (M+1)⁺.

### Intermediate A-28

### ethyl 4-benzyloxy-2-chloro-6-methyl-5-oxamoyl-pyridine-3-carboxylate

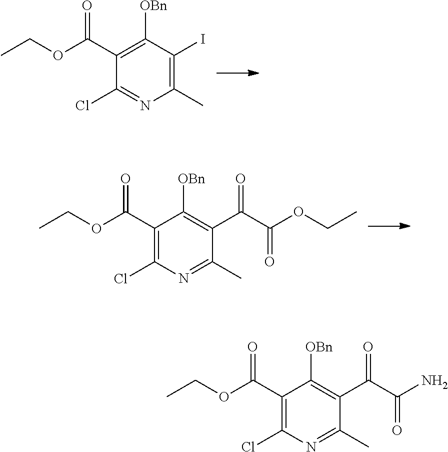

### Step 1: ethyl 4-benzyloxy-2-chloro-5-(2-ethoxy-2-oxo-acetyl)-6-methyl-pyridine-3-carboxylate

Ethyl 4-benzyloxy-2-chloro-5-iodo-6-methyl-pyridine-3-carboxylate (Preparation 1, 408 mg, 0.945 mmol) was dissolved in Et₂O (7 mL). The resulting mixture was cooled to −78° C. n-BuLi (500 μL of 2.5 M, 1.3 mmol) was added dropwise over 5 min and the resulting mixture was stirred at −78° C. for 25 min. Ethyl 2-chloro-2-oxo-acetate (340 μL, 3.04 mmol) was added dropwise and the resulting mixture was allowed to stir for 30 min at −78° C. After 30 min the reaction was placed into a 0° C. bath and quenched with a saturated aqueous solution of ammonium chloride (5 mL). The mixture was diluted with ethyl acetate and then washed with brine. The organic layer was dried over magnesium sulfate, filtered, and then concentrated under reduced pressure. Purification by silica gel chromatography (0-40% ethyl acetate/hexanes over 20 min) provided ethyl 4-benzyloxy-2-chloro-5-(2-ethoxy-2-oxo-acetyl)-6-methyl-pyridine-3-carboxylate (200 mg, 50%). ¹H NMR (400 MHz, DMSO-d₆) δ 7.53-7.23 (m, 5H), 5.08 (s, 2H), 4.41 (q, J=7.1 Hz, 2H), 3.95 (q, J=7.1 Hz, 2H), 2.46 (s, 3H), 1.30 (t, J=7.1 Hz, 3H), 1.05 (t, J=7.1 Hz, 3H). ESI-MS m/z calc. 405.10, found 406.4 (M+1)⁺.

### Step 2: ethyl 4-benzyloxy-2-chloro-6-methyl-5-oxamoyl-pyridine-3-carboxylate

A solution of ethyl 4-benzyloxy-2-chloro-5-(2-ethoxy-2-oxo-acetyl)-6-methyl-pyridine-3-carboxylate (200 mg, 0.468 mmol) in ethanol (1 mL) at 0° C. was treated dropwise with ammonia in methanol (550 μL of 2.0 M, 1.1 mmol) then stirred at room temperature for 3 h. The solvent was removed in vacuo to provide ethyl 4-benzyloxy-2-chloro-6-methyl-5-oxamoyl-pyridine-3-carboxylate (166 mg, 89%). ¹H NMR (400 MHz, DMSO-d₆) δ 8.43 (s, 1H), 8.08 (s, 1H), 7.47-7.27 (m, 5H), 5.00 (s, 2H), 4.36 (q, J=7.1 Hz, 2H), 2.37 (s, 3H), 1.27 (t, J=7.1 Hz, 3H). ESI-MS m/z calc. 376.08, found 377.4 (M+1)⁺.

### Intermediate A-29

### ethyl 5-(2-amino-1-hydroxy-2-oxo-ethyl)-4-benzyloxy-2-chloro-6-methyl-pyridine-3-carboxylate

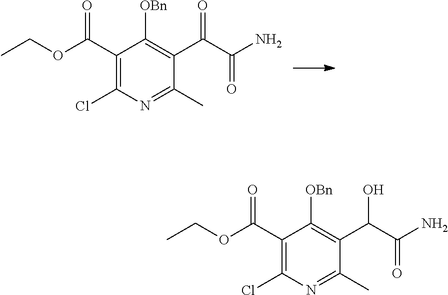 [SMILES: CCOC(=O)c1c(Cl)nc(C)c(C(=O)C(N)=O)c1OCc1ccccc1 ; CCOC(=O)c1c(Cl)nc(C)c(C(O)C(N)=O)c1OCc1ccccc1]

A solution of ethyl 4-benzyloxy-2-chloro-6-methyl-5-oxamoyl-pyridine-3-carboxylate (135 mg, 0.340 mmol) in ethanol (1 mL) and THF (1 mL) at 0° C. was treated with sodium borohydride (14 mg, 0.37 mmol) and stirred at room temperature for 10 min. The mixture was quenched with a saturated aqueous solution of ammonium chloride (5 mL), diluted with ethyl acetate and then washed with brine. The organic layer was dried over magnesium sulfate, filtered, and then concentrated under reduced pressure to provide ethyl 5-(2-amino-1-hydroxy-2-oxo-ethyl)-4-benzyloxy-2-chloro-6-methyl-pyridine-3-carboxylate (128 mg, 99%). ¹H NMR (400 MHz, DMSO-d₆) δ 7.63 (br s, 1H), 7.60 (br s, 1H), 7.48-7.36 (m, 5H), 6.49 (d, J=5.2 Hz, 1H), 5.40 (d, J=4.9 Hz, 1H), 5.14 (d, J=10.7 Hz, 1H), 5.02 (d, J=10.6 Hz, 1H), 4.41-4.27 (m, 2H), 2.50 (s, 3H) overlaps with DMSO, 1.25 (t. J=7.1 Hz, 3H). ESI-MS m/z calc. 378.10, found 379.3 (M+1)⁺.

### Intermediate A-30

### ethyl 5-(2-amino-1-methoxy-2-oxo-ethyl)-4-benzyloxy-2-chloro-6-methyl-pyridine-3-carboxylate

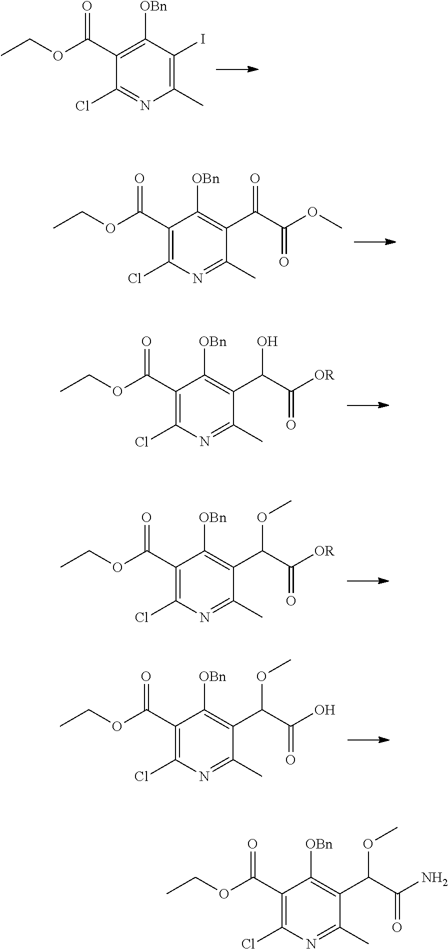

### Step 1: ethyl 4-benzyloxy-2-chloro-5-(2-methoxy-2-oxo-acetyl)-6-methyl-pyridine-3-carboxylate

Ethyl 4-benzyloxy-2-chloro-5-iodo-6-methyl-pyridine-3-carboxylate (Preparation 1, 539 mg, 1.25 mmol) was dissolved in Et₂O (8 mL). The resulting mixture was cooled to −78° C. n-BuLi (650 μL of 2.5 M, 1.63 mmol) was added dropwise over 5 min and the resulting mixture was stirred at −78° C. for 25 min. Methyl 2-chloro-2-oxo-acetate (350 μL, 3.80 mmol) was then added dropwise and the resulting mixture was allowed to stir for 30 min at −78° C. After 30 min the reaction was placed into a 0° C. bath and quenched with a saturated aqueous solution of ammonium chloride (5 mL), diluted with ethyl acetate and then washed with brine. The organic layer was dried over anhydrous magnesium sulfate, filtered, and then concentrated under reduced pressure. Purification by silica gel chromatography (0-40% of ethyl acetate/hexanes over 20 min) provided ethyl 4-benzyloxy-2-chloro-5-(2-methoxy-2-oxo-acetyl)-6-methyl-pyridine-3-carboxylate (300 mg, 61%). ¹H NMR (400 MHz, DMSO-d₆) δ 7.47-7.36 (m, 3H), 7.36-7.29 (m, 2H), 5.09 (s, 2H), 4.42 (q, J=7.1 Hz, 2H), 3.50 (s, 3H), 2.46 (s, 3H), 1.31 (t, J=7.1 Hz, 3H). ESI-MS m/z calc. 391.08, found 392.3 (M+1)⁺.

### Step 2: ethyl 4-benzyloxy-2-chloro-5-(2-ethoxy-1-hydroxy-2-oxo-ethyl)-6-methyl-pyridine-3-carboxylate and ethyl 4-benzyloxy-2-chloro-5-(1-hydroxy-2-methoxy-2-oxo-ethyl)-6-methyl-pyridine-3-carboxylate

Ethyl 4-benzyloxy-2-chloro-5-(2-methoxy-2-oxo-acetyl)-6-methyl-pyridine-3-carboxylate (300 mg, 0.766 mmol) was dissolved in ethanol (1.5 mL) and THF (1.5 mL). The resulting mixture was cooled to 0° C. Then sodium borohydride (20 mg, 0.53 mmol) was added and the resulting mixture was stirred at room temperature for 10 min. The mixture was quenched with a saturated aqueous ammonium chloride (5 mL), diluted with ethyl acetate and then washed with brine. The organic layer was dried over magnesium sulfate, filtered, and then concentrated under reduced pressure to yield a mixture of the major ethyl ester trans-esterification product: ethyl 4-benzyloxy-2-chloro-5-(2-ethoxy-1-hydroxy-2-oxo-ethyl)-6-methyl-pyridine-3-carboxylate. ESI-MS m/z calc. 407.11, found 408.55 (M+1)⁺ and the minor methyl ester product: ethyl 4-benzyloxy-2-chloro-5-(1-hydroxy-2-methoxy-2-oxo-ethyl)-6-methyl-pyridine-3-carboxylate. ESI-MS m/z calc. 393.10, found 394.46 (M+1)⁻.

Step 3: A solution of the mixture from step 2 in DMF (3 mL) was cooled to 0° C. and treated with sodium hydride (33 mg, 60% dispersion in mineral oil, 1.4 mmol) at 0° C. for 5 min. Methyl iodide (150 μL, 2.41 mmol) was then added and the reaction gradually warmed to room temperature and stirred for 30 min. The mixture was diluted with ethyl acetate, washed with a saturated aqueous solution of ammonium chloride and then brine. The organic layer was dried over magnesium sulfate, filtered, and concentrated under reduced pressure to provide a mixture of the major product ethyl 4-benzyloxy-2-chloro-5-(2-ethoxy-1-methoxy-2-oxo-ethyl)-6-methyl-pyridine-3-carboxylate, ESI-MS m/z calc. 421.13, found 422.58 (M+1)⁺ and minor product ethyl 4-benzyloxy-2-chloro-5-(1,2-dimethoxy-2-oxo-ethyl)-6-methyl-pyridine-3-carboxylate. ESI-MS m/z calc. 407.11, found 408.38 (M+1)⁺.

### Step 4: 2-(4-benzyloxy-6-chloro-5-ethoxycarbonyl-2-methyl-3-pyridyl)-2-methoxy-acetic acid

The mixture from step 3 was dissolved in ethanol (5 mL) and treated with aqueous NaOH (1.5 mL of 1 M, 1.5 mmol) and stirred at 25° C. for 40 min under nitrogen. The mixture was acidified using 1 M aqueous HCl to pH<5 and diluted with ethyl acetate. The organic phase was washed with saturated aqueous ammonium chloride and brine. The organic layer was dried over magnesium sulfate, filtered, and concentrated under reduced pressure to provide 2-(4-benzyloxy-6-chloro-5-ethoxycarbonyl-2-methyl-3-pyridyl)-2-methoxy-acetic acid (250 mg, 77%). ¹H NMR (400 MHz, DMSO-d₆) δ 13.13 (br s, 1H), 7.46-7.34 (m, 5H), 5.18 (d, J=10.7 Hz, 1H) overlaps with 5.18 (s, 1H), 4.95 (d, J=10.7 Hz, 1H), 4.32 (q, J=7.1 Hz, 2H), 3.35 (s, 3H) overlaps with HOH, 2.51 (s, 3H) overlaps with DMSO, 1.24 (t, J=7.1 Hz, 3H). ESI-MS m/z calc. 393.10, found 394.18 (M+1)⁺.

### Step 5: ethyl 5-(2-amino-1-methoxy-2-oxo-ethyl)-4-benzyloxy-2-chloro-6-methyl-pyridine-3-carboxylate

A solution of 2-(4-benzyloxy-6-chloro-5-ethoxycarbonyl-2-methyl-3-pyridyl)-2-methoxy-acetic acid (250 mg, 0.590 mmol) in DMF (1 mL) was treated with HATU (225 mg, 0.592 mmol), DIPEA (110 μL, 0.632 mmol) and NH₃ in methanol (200 μL of 7 M, 1.4 mmol) then stirred for 20 min. The mixture was diluted with ethyl acetate and washed with saturated aqueous ammonium chloride and brine. The organic layer was dried over anhydrous magnesium sulfate, filtered, and concentrated under reduced pressure. Purification by silica gel chromatography (0-100% ethyl acetate/heptanes over 25 min) provided ethyl 5-(2-amino-1-methoxy-2-oxo-ethyl)-4-benzyloxy-2-chloro-6-methyl-pyridine-3-carboxylate (187 mg, 81%). ¹H NMR (400 MHz, DMSO-d₆) δ 7.63 (br s, 1H), 7.61 (br s, 1H), 7.47-7.34 (m, 5H), 5.15 (d, J=10.8 Hz, 1H), 5.11 (s, 1H), 4.99 (d, J=10.8 Hz, 1H), 4.38-4.26 (m, 2H), 3.21 (s, 3H), 2.47 (s, 3H), 1.25 (t, J=7.1 Hz, 3H). ESI-MS m/z calc. 392.11, found 393.4 (M+1)⁺.

### Intermediate A-31

### 2-(4-benzyloxy-6-chloro-5-ethoxycarbonyl-2-methyl-3-pyridyl)acetic acid

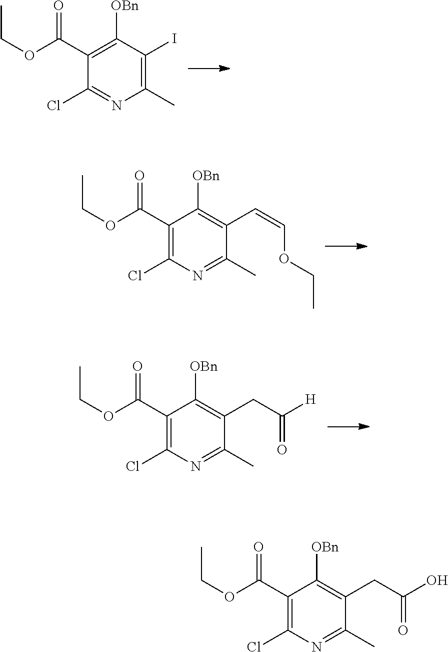 [SMILES: CCOC(=O)c1c(Cl)nc(C)c(I)c1OCc1ccccc1 ; CCO/C=C\c1c(C)nc(Cl)c(C(=O)OCC)c1OCc1ccccc1 ; CCOC(=O)c1c(Cl)nc(C)c(CC=O)c1OCc1ccccc1 ; CCOC(=O)c1c(Cl)nc(C)c(CC(=O)O)c1OCc1ccccc1]

### Step 1: ethyl 4-benzyloxy-2-chloro-5-[(Z)-2-ethoxyvinyl]-6-methyl-pyridine-3-carboxylate

A mixture of ethyl 4-benzyloxy-2-chloro-5-iodo-6-methyl-pyridine-3-carboxylate (Preparation 1, 573 mg, 1.327 mmol), tributyl-[(Z)-2-ethoxyvinyl]stannane (593 mg, 1.642 mmol) and PdCl₂(PPh₃)₂ (182 mg, 0.260 mmol) in toluene (5 mL) was degassed for 5 min and placed under a nitrogen atmosphere. The tube was sealed and stirred at 110° C. for 20 h. The mixture was diluted with ethyl acetate, washed with saturated aqueous ammonium chloride and brine. The organic layer was dried over magnesium sulfate, filtered, and then concentrated under reduced pressure. Purification by silica gel chromatography (0-50% ethyl acetate/heptanes over 15 min) provided ethyl 4-benzyloxy-2-chloro-5-[(Z)-2-ethoxyvinyl]-6-methyl-pyridine-3-carboxylate (235 mg, 47%). ¹H NMR (400 MHz, DMSO-d₆) δ 7.45-7.32 (m, 5H), 6.58 (d, J=6.8 Hz, 1H), 5.32 (d, J=6.8 Hz, 1H), 5.04 (s, 2H), 4.27 (q, J=7.1 Hz, 2H), 3.94 (q, J=7.0 Hz, 2H), 2.41 (s, 3H), 1.21 (t, J=7.1 Hz, 3H), 1.17 (t, J=7.1 Hz, 3H). ESI-MS m/z calc. 375.12, found 376.1 (M+1)⁺.

### Step 2: ethyl 4-benzyloxy-2-chloro-6-methyl-5-(2-oxoethyl)pyridine-3-carboxylate

To a solution of ethyl 4-benzyloxy-2-chloro-5-[(Z)-2-ethoxyvinyl]-6-methyl-pyridine-3-carboxylate from step 1 in acetone (4 mL) was added aqueous HCl (2.5 mL of 1 M, 2.500 mmol) and the mixture stirred at 50° C. for 3 h. The mixture was diluted with ethyl acetate, washed with a saturated aqueous ammonium chloride and brine. The organic layer was dried over magnesium sulfate, filtered, and then concentrated under reduced pressure to provide ethyl 4-benzyloxy-2-chloro-6-methyl-5-(2-oxoethyl)pyridine-3-carboxylate was used directly in step 3.

### Step 3: 2-(4-benzyloxy-6-chloro-5-ethoxycarbonyl-2-methyl-3-pyridyl)acetic acid

A solution of crude ethyl 4-benzyloxy-2-chloro-6-methyl-5-(2-oxoethyl)pyridine-3-carboxylate from step 2 was in DMF (3 mL) was treated with OXONE (813 mg, 1.32 mmol) and stirred at room temperature for 1 hour. The mixture was diluted with ethyl acetate, washed with a saturated aqueous ammonium chloride and brine. The organic layer was dried over magnesium sulfate, filtered, and then concentrated under reduced pressure. Purification by silica gel chromatography (0-50% ethyl acetate/heptanes over 20 min) provided 2-(4-benzyloxy-6-chloro-5-ethoxycarbonyl-2-methyl-3-pyridyl)acetic acid (285 mg, 59%). ¹H NMR (400 MHz, DMSO-d₆) δ 12.72 (br s, 1H), 7.48-7.35 (m, 5H), 5.00 (s, 2H), 4.35 (q, J=7.1 Hz, 2H), 3.67 (s, 2H), 2.44 (s, 3H), 1.27 (t, J=7.1 Hz, 3H). ESI-MS m/z calc. 363.09, found 364.4 (M+1)⁺.

### Intermediate A-32

### ethyl 4-benzyloxy-2-chloro-5-(2-methoxy-2-oxo-ethyl)-6-methyl-pyridine-3-carboxylate

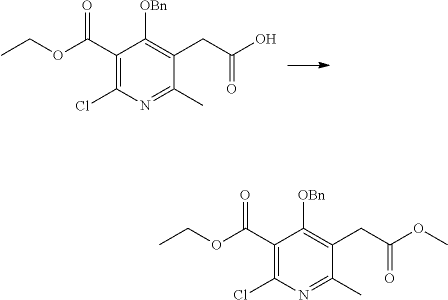 [SMILES: CCOC(=O)c1c(Cl)nc(C)c(CC(=O)O)c1OCc1ccccc1 ; CCOC(=O)c1c(Cl)nc(C)c(CC(=O)OC)c1OCc1ccccc1]

A solution of 2-(4-benzyloxy-6-chloro-5-ethoxycarbonyl-2-methyl-3-pyridyl)acetic acid (285 mg, 0.783 mmol) in DCM (8 mL) at 0° C. was treated with oxalyl dichloride (600 μL of 2 M in DCM, 1.2 mmol) followed by DMF (6 μL, 0.08 mmol). The resulting mixture was stirred for 30 min at 0° C., then treated slowly with DIPEA (150 μL, 0.861 mmol) followed by methanol (1 mL, 25 mmol). The mixture was stirred for 30 min, then diluted with ethyl acetate. The organic phase was washed with saturated aqueous ammonium chloride and brine, dried over magnesium sulfate, filtered, and concentrated under reduced pressure. Purification by silica gel chromatography (0-50% of ethyl acetate/heptanes over 20 min) provided ethyl 4-benzyloxy-2-chloro-5-(2-methoxy-2-oxo-ethyl)-6-methyl-pyridine-3-carboxylate (220 mg, 74%). ¹H NMR (400 MHz, DMSO-d₆) δ 7.47-7.34 (m, 5H), 5.00 (s, 2H), 4.36 (q, J=7.1 Hz, 2H), 3.74 (s, 2H), 3.58 (s, 3H), 2.43 (s, 3H), 1.28 (t, J=7.1 Hz, 3H). ESI-MS m/z calc. 377.82, found 379.3 (M+1)⁺.

### Intermediate A-33

### ethyl 4-benzyloxy-2-chloro-5-(2-methoxy-1-methyl-2-oxo-ethyl)-6-methyl-pyridine-3-carboxylate

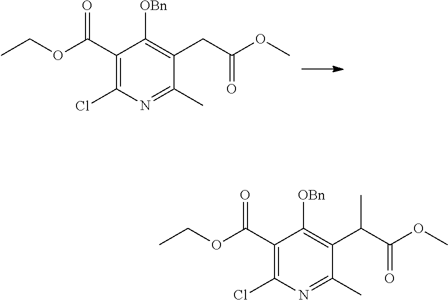

A solution of ethyl 4-benzyloxy-2-chloro-5-(2-methoxy-2-oxo-ethyl)-6-methyl-pyridine-3-carboxylate (220 mg, 0.577 mmol) in DMF (3 mL) at 0° C. was treated with sodium hydride (43 mg, 60% dispersion in mineral oil, 1.1 mmol) at 0° C. for 5 min. MeI (150 μL, 2.41 mmol) was added and the reaction gradually warmed to room temperature and stirred for 30 min. The mixture was diluted with ethyl acetate, washed with saturated aqueous ammonium chloride and brine. The organic layer was dried over magnesium sulfate, filtered, and concentrated under reduced pressure to provide ethyl 4-benzyloxy-2-chloro-5-(2-methoxy-1-methyl-2-oxo-ethyl)-6-methyl-pyridine-3-carboxylate. ESI-MS m/z calc. 391.12, found 392.53 (M+1)⁺.

### Intermediate A-34

### ethyl 5-(2-amino-1-methyl-2-oxo-ethyl)-4-benzyloxy-2-chloro-6-methyl-pyridine-3-carboxylate

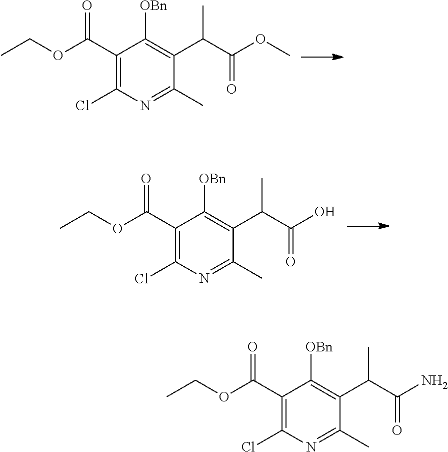

### Step 1: 2-(4-benzyloxy-6-chloro-5-ethoxycarbonyl-2-methyl-3-pyridyl)propanoic acid

A solution of ethyl 4-benzyloxy-2-chloro-5-(2-methoxy-1-methyl-2-oxo-ethyl)-6-methyl-pyridine-3-carboxylate (210 mg, 0.536 mmol) in ethanol (6 mL) was treated with aqueous NaOH (1 mL of 1 M, 1.0 mmol) and stirred under nitrogen atmosphere at 45° C. for 6 h. The mixture was acidified using aqueous 1 M HCl to pH<5 and diluted with ethyl acetate. The organic layer was washed with a saturated aqueous ammonium chloride and brine. The organic layer was dried over magnesium sulfate, filtered, and concentrated under reduced pressure to provide 2-(4-benzyloxy-6-chloro-5-ethoxycarbonyl-2-methyl-3-pyridyl)propanoic acid. ESI-MS m/z calc. 377.10, found 378.4 (M+1)⁺.

### Step 2: ethyl 5-(2-amino-1-methyl-2-oxo-ethyl)-4-benzyloxy-2-chloro-6-methyl-pyridine-3-carboxylate

A solution of 2-(4-benzyloxy-6-chloro-5-ethoxycarbonyl-2-methyl-3-pyridyl)propanoic acid in DMF (1 mL) from step 1 was treated with HATU (200 mg, 0.526 mmol), DIPEA (100 μL, 0.574 mmol) and NH₃ in methanol (150 μL of 7 M, 1.1 mmol) and stirred for 30 min. The mixture was diluted with ethyl acetate, washed with saturated aqueous ammonium chloride and brine. The organic layer was dried over magnesium sulfate, filtered, and concentrated under reduced pressure. Purification by silica gel chromatography (0-100% of ethyl acetate/heptanes over 25 min) provided ethyl 5-(2-amino-1-methyl-2-oxo-ethyl)-4-benzyloxy-2-chloro-6-methyl-pyridine-3-carboxylate (214 mg, 99%). ¹H NMR (400 MHz, DMSO-d₆) δ 7.47-7.33 (m, 5H), 7.11 (br s, 1H), 7.05 (br s, 1H), 5.08-4.97 (m, 2H), 4.38-4.26 (m, 2H), 3.92 (q, J=7.2 Hz, 1H), 2.43 (s, 3H), 1.28 (d, J=7.2 Hz, 3H) overlaps with 1.25 (t, J=7.1 Hz, 3H). ESI-MS m/z calc. 376.12, found 377.23 (M+1)⁺.

### Intermediate A-35

### 2-(4-benzyloxy-6-chloro-2-methyl-3-pyridyl)-N-methyl-acetamide

### Step 1: 2-(4-benzyloxy-6-chloro-2-methyl-3-pyridyl)-N-methyl-acetamide

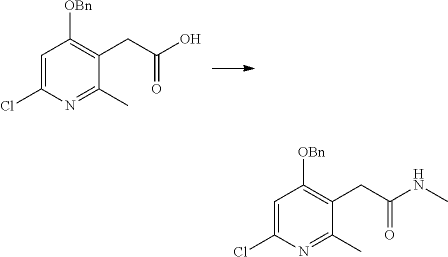 [SMILES: Cc1nc(Cl)cc(OCc2ccccc2)c1CC(=O)O ; CNC(=O)Cc1c(OCc2ccccc2)cc(Cl)nc1C]

A solution of 2-(4-Benzyloxy-6-chloro-2-methyl-3-pyridyl)acetic acid (Intermediate A-26, step 3, 73 mg, 0.25 mmol) was dissolved in DMF (750 μL), then HATU (110 mg, 0.29 mmol), DIPEA (65 μL, 0.37 mmol), and MeNH₂ in ethanol (120 μL of 33% w/v, 1.28 mmol) were added and the resulting mixture was stirred for 30 min. The resulting mixture was diluted with ethyl acetate, washed with a saturated aqueous solution of ammonium chloride and then brine. The organic layer was dried over anhydrous magnesium sulfate, filtered, and concentrated under reduced pressure. Purification by silica gel chromatography (0-100% ethyl acetate/hexanes) gave 2-(4-benzyloxy-6-chloro-2-methyl-3-pyridyl)-N-methyl-acetamide (70.1 mg, 92%). ESI-MS m/z calc. 304.1, found 305.3 (M+1)⁺. ¹H NMR (400 MHz, DMSO-d₆) δ 7.79 (q, J=4.8 Hz, 1H), 7.45-7.29 (m, 5H), 7.06 (s, 1H), 5.22 (s, 2H), 3.46 (s, 2H), 2.56 (d, J=4.6 Hz, 3H), 2.34 (s, 3H).

### Intermediate A-36

### 4-benzyloxy-2-chloro-6-methyl-3-methylsulfanyl-pyridine

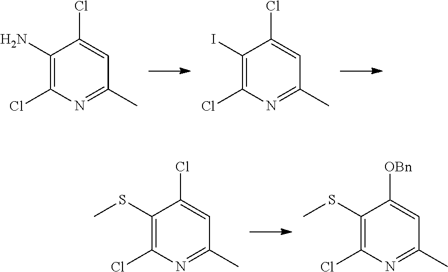

### Step 1: 2,4-dichloro-3-iodo-6-methyl-pyridine

2,4-Dichloro-6-methyl-pyridin-3-amine (250 mg, 1.41 mmol) and p-TsOH hydrate (806 mg, 4.24 mmol) were suspended in acetonitrile (5.6 mL) and cooled to 0° C. In a separate flask sodium nitrite (195 mg, 2.83 mmol) and potassium iodide (586 mg, 3.53 mmol) were dissolved in water (0.84 mL). The aqueous NaNO₂/KI mixture was added dropwise to the amine/p-TsOH mixture at 0° C. The resulting mixture was allowed to warm to room temperature and stirred for 2 h. The mixture was poured over water (20 mL) and diluted with TBME (20 mL). The organic layer was separated and the aqueous layer was extracted with TBME (2×10 mL). The combined organic extracts were washed with brine (20 mL), dried over magnesium sulfate, filtered and concentrated in vacuo. Purification by silica gel chromatography (24 g silica, 0-20% ethyl acetate/heptane) provided 2,4-dichloro-3-iodo-6-methyl-pyridine (223 mg, 55%) as a pale yellow oil. ESI-MS m/z calc. 286.88, found 288.2 (M+1)⁺. ¹H NMR (400 MHz, CDCl₃) δ 7.16 (d, J=0.6 Hz, 1H), 1.56 (s, 3H).

### Step 2: 2,4-dichloro-6-methyl-3-methylsulfanyl-pyridine

2,4-Dichloro-3-iodo-6-methyl-pyridine (200 mg, 0.695 mmol), Pd₂(dba)₃ (16 mg, 0.018 mmol) and Xantphos (20 mg, 0.035 mmol) were added to a vial which was purged with N₂. 1,4-dioxane (2.5 mL), DIPEA (250 μL, 1.44 mmol) and sodium thiomethoxide (230 μL of 21% w/v in water, 0.6891 mmol) were added and the resulting mixture was stirred at 100° C. for 45 min. The mixture was poured over water (20 mL) and diluted with TBME (20 mL). The organic layer was separated and the aqueous layer was extracted with TBME (20 mL). The combined organic extracts were washed with brine, dried over magnesium sulfate and concentrated in vacuo. Purification by silica gel chromatography (24 g silica, 0-20% ethyl acetate/heptane) provided 2,4-dichloro-6-methyl-3-methylsulfanyl-pyridine (77 mg, 53%) as a yellow oil. ESI-MS w % z calc. 206.97, found 208.2 (M+1)⁺. ¹H NMR (400 MHz, CDCl₃) δ 7.21 (d, J=0.5 Hz, 1H), 2.50 (d, J=0.5 Hz, 3H), 2.44 (s, 3H).

### Step 3: 4-benzyloxy-2-chloro-6-methyl-3-methylsulfanyl-pyridine

4-Benzyloxy-2-chloro-6-methyl-3-methylsulfanyl-pyridine was prepared from_2,4-dichloro-6-methyl-3-methylsulfanyl-pyridine and benzyl alcohol using a procedure analogous to that found in Preparation 1, step 3 and NMP as the solvent. ESI-MS m/z calc. 279.05, found 280.4 (M+1)⁺. ¹H NMR (400 MHz, CDCl₃) δ 7.46-7.35 (m, 5H), 6.67 (s, 1H), 5.20 (s, 2H), 2.47 (d, J=0.5 Hz, 3H), 2.38 (s, 3H).

### Intermediate A-37

### N-benzyl-4-benzyloxy-6-chloro-N,2-dimethyl-pyridine-3-sulfonamide

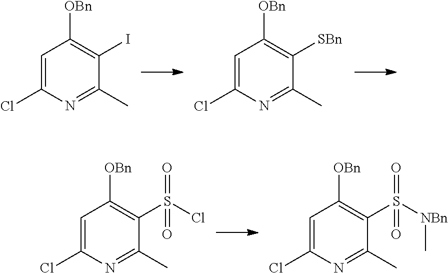

### Step 1: 4-benzyloxy-3-benzylsulfanyl-6-chloro-2-methyl-pyridine

To a solution of 4-benzyloxy-6-chloro-3-iodo-2-methyl-pyridine (Preparation 2, 1.5 g, 4.2 mmol) in 1,4-dioxane (30 mL) was added phenylmethanethiol (640 mg, 0.6 mL, 5.1 mmol) followed by DIPEA (1.1 g, 1.5 mL, 8.6 mmol). The solution was purged with argon, treated with Xantphos (240 mg, 0.415 mmol) and Pd₂(dba)₃ (190 mg, 0.208 mmol) then heated at 80° C. for 2 h. The mixture was filtered through a Celite® pad and concentrated under vacuum. Purification by silica gel chromatography (10-12% ethyl acetate/hexanes) provided 4-benzyloxy-3-benzylsulfanyl-6-chloro-2-methyl-pyridine (810 mg, 55%). ESI-MS m/z calc. 355.08, found 356.08 (M+1)⁺.

### Step 2: 4-benzyloxy-6-chloro-2-methyl-pyridine-3-sulfonyl chloride

To a solution of 4-benzyloxy-3-benzylsulfanyl-6-chloro-2-methyl-pyridine (800 mg, 2.25 mmol) in acetonitrile (25 mL), AcOH (1 mL) and water (0.5 mL) at 0° C. was added 1,3-dichloro-5,5-dimethyl-imidazolidine-2,4-dione (900 mg, 4.57 mmol) portion-wise. The mixture was stirred for 2 h at 0-5° C. then concentrated under vacuum. The residue was diluted with DCM (30 mL) and the solution cooled to 0° C. A solution of 5% saturated aqueous sodium bicarbonate (30 mL) was added slowly (maintaining temperature<10° C.) and stirred at 0-5° C. for 15 min. The organic layer was separated, washed with brine (10 mL), dried over sodium sulfate and concentrated in vacuo to afford 4-benzyloxy-6-chloro-2-methyl-pyridine-3-sulfonyl chloride (520 mg, 70%) as white solid.

### Step 3: N-benzyl-4-benzyloxy-6-chloro-N,2-dimethyl-pyridine-3-sulfonamide

To a solution of 4-benzyloxy-6-chloro-2-methyl-pyridine-3-sulfonyl chloride (250 mg, 0.753 mmol) in DCM (5 mL) was added pyridine (0.3 mL, 3.7 mmol) followed by N-methylbenzylamine (110 mg, 0.908 mmol). The mixture was stirred at room temperature for 5 h, then quenched with water (20 mL) and extracted with DCM (2×25 mL). The combined organic layers were washed with brine (20 mL), dried over sodium sulfate and concentrated in vacuo. Purification by silica gel chromatography (5-8% ethyl acetate/hexanes) afforded N-benzyl-4-benzyloxy-6-chloro-N,2-dimethyl-pyridine-3-sulfonamide (142 mg, 43%). ¹H NMR (400 MHz, CDCl₃) δ 7.48-7.35 (m, 5H), 7.31-7.19 (m, 2H), 7.11-7.04 (m, 2H), 6.91 (s, 1H), 5.17 (s, 2H), 4.04 (s, 2H), 2.88 (s, 3H), 2.54 (s, 3H). (1H missing, likely under solvent signal). ESI-MS m/z calc. 416.09, found 417.33 (M+1)⁺.

### Intermediate A-38

### N,N-dibenzyl-4-benzyloxy-6-chloro-2-methyl-pyridine-3-sulfonamide

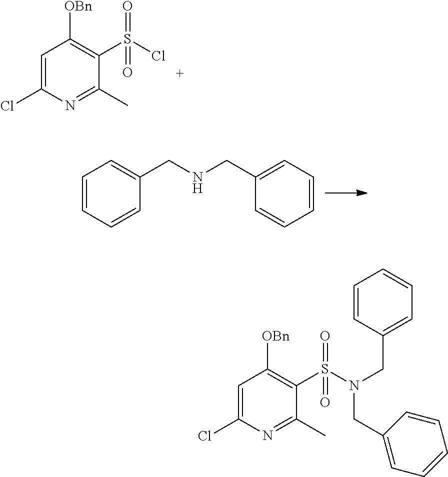 [SMILES: Cc1nc(Cl)cc(OCc2ccccc2)c1S(=O)(=O)Cl ; c1ccc(CNCc2ccccc2)cc1 ; Cc1nc(Cl)cc(OCc2ccccc2)c1S(=O)(=O)N(Cc1ccccc1)Cc1ccccc1]

N,N-Dibenzyl-4-benzyloxy-6-chloro-2-methyl-pyridine-3-sulfonamide is prepared from 4-benzyloxy-6-chloro-2-methyl-pyridine-3-sulfonyl chloride and dibenzylamine using a procedure analogous to that used to prepare Intermediate A-37. ¹H NMR (400 MHz, DMSO-d₆) δ 7.42 (s, 5H), 7.36 (s, 1H), 7.23 (h, J=3.6 Hz, 6H), 6.89 (dd, J=7.2, 2.4 Hz, 4H), 5.22 (s, 2H), 4.03 (s, 4H), 2.71 (s, 3H). ESI-MS m/z calc. 492.13, found 493.33 (M+1)⁺.

### General Scheme for the Preparation of Intermediate A

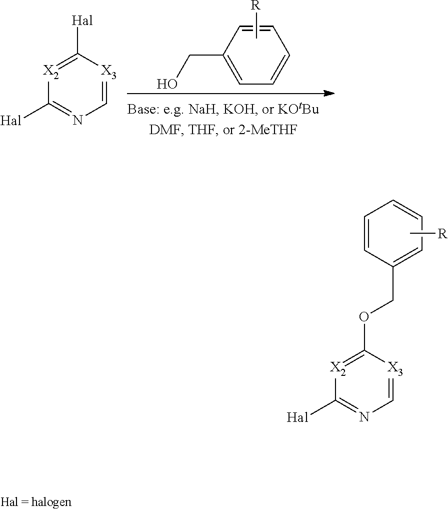

Intermediates in Table 1 were prepared using the corresponding dichloro-pyridine or -pyrimidines and a procedure analogous to that found in Preparation 1, step 3. Dichloro-pyridines or -pyrimidines were obtained from commercial sources. Benzyl alcohol or substituted benzyl alcohols, such as 2-methoxy benzyl alcohol, may be used. DMF, THF, 2-MeTHF, or mixtures of these solvents may be used as the reaction solvent.

TABLE 1

| | | MW & | |
|---|---|---|---|
| | Compound | Found | |
| Intermediate | Name | [M + H]⁺ | NMR (shifts in ppm) |
| Intermediate A - | ethyl 6-chloro-4-[(4- | 335.09, | ¹H NMR (500 MHz, DMSO-d₆) δ 7.40- |
| 39 | methoxyphenyl)methoxy]- | 336.2 | 7.32 (m, 2H), 7.28 (s, 1H), 7.02-6.92 |
| | 2-methyl-pyridinc-3- | | (m, 2H), 5.21 (s, 2H), 4.29 (q, J = 7.1 |
| | carboxylate | | Hz, 2H), 3.76 (s, 3H), 2.35 (s, 3H), 1.21 |
| | | | (t, J = 7.1 Hz, 3H). |
| Intermediate A - | ethyl 4-benzyloxy-2- | 305.08, | ¹H NMR (400 MHz, DMSO-d₆) δ 7.45- |
| 40 | chloro-6-methyl-pyridine- | 306.3 | 7.38 (m, 4H), 7.38-7.33 (m, 1H), 7.26 |
| | 3-carboxylate | | (s, 1H), 5.29 (s, 2H), 4.30 (q, J = 7.1 Hz, |
| | | | 2H), 2.44 (s, 3H), 1.22 (t, J = 7.1 Hz, |
| | | | 3H). |
| Intermediate A - | 4-benzyloxy-6-chloro-2,3- | 247.08, | ¹H NMR (400 MHz, CDCl₃) δ 7.46- |
| 41 | dimethyl-pyridine | 248.1 | 7.33 (m, 5H), 6.72 (s, 1H), 5.09 (s, 2H), |
| | | | 2.46 (s, 3H), 2.15 (s, 3H). |
| Intermediate A - | 4-benzyloxy-6-chloro-3- | 263.07 | ¹H NMR (400 MHz, CDCl₃) δ 7.44- |
| 42 | methoxy-2-methyl- | 264.2 | 7.34 (m, 5H), 6.78 (s, 1H), 5.13 (s, 2H), |
| | pyridine | | 3.82 (s, 3H), 2.44 (s, 3H). |
| Intermediate A - | 4-benzyloxy-2-chloro-6- | 233.06 | ¹H NMR (400 MHz, CDCl₃) δ 7.46- |
| 43 | methyl-pyridine | 234.1 | 7.34 (m, 5H), 6.76 (d, J = 1.6 Hz, 1H), |
| | | | 6.68 (d, J = 1.6 Hz, 1H), 5.09 (s, 2H), |
| | | | 2.48 (s, 3H). |
| Intermediate A - | ethyl 6-chloro-4-[(4- | 335.09 | ¹H NMR (500 MHz, DMSO-d₆) δ 7.40- |
| 44 | methoxyphenyl)methoxy]- | 336.2 | 7.32 (m, 2H), 7.28 (s, 1H), 7.02-6.92 |
| | 2-methyl-pyridine-3- | | (m, 2H), 5.21 (s, 2H), 4.29 (q, J = 7.1 |
| | carboxylate | | Hz, 2H), 3.76 (s, 3H), 2.35 (s, 3H), 1.21 |
| | | | (t, J = 7.1 Hz, 3H). |
| Intermediate A - | 4-benzyloxy-2-chloro-6- | 249.05 | ¹H NMR (400 MHz, CDCl₃) δ 7.40 (d, |
| 45 | methoxy-pyridine | 250.2 | J = 5.8 Hz, 5H), 6.58 (d, J = 1.9 Hz, |
| | | | 1H), 6.19 (d, J = 1.9 Hz, 1H), 5.06 (s, |
| | | | 2H), 3.91 (s, 3H). |
| Intermediate A - | 2-chloro-4-1(4- | 277.08 | ¹H NMR (500 MHz, CDCl₃) δ 7.36- |
| 46 | methoxyphenyl)methoxy]- | 278.4 | 7.29 (m, 2H), 6.97-6.90 (m, 2H), 6.64 |
| | 3,6-dimethyl-pyridine | | (s, 1H), 5.03 (s, 2H), 3.83 (s, 3H), 2.46 |
| | | | (s, 3H), 2.23 (s, 3H). |
| Intermediate A - | 4-benzyloxy-2-chloro-6- | 234.06, | ¹H NMR (400 MHz, CDCl₃): 7.39- |
| 47 | methyl-pyrimidine | 235.2 | 7.25 (m, 5H), 6.46 (s, 1H), 5.34 (s, 2H), |
| | | | 2.35 (s, 3H). |
| Intermediate A - | 4-benzyloxy-6-chloro-2- | 234.06, | ¹H NMR (400 MHz, CDCl₃) δ 7.48- |
| 48 | methyl-pyrimidine | 235.1 | 7.31 (m, 5H), 6.63 (s, 1H), 5.43 (s, 2H), |
| | | | 2.63 (s, 3H). |
| Intermediate A - | 4-benzyloxy-6-chloro-2,5- | 248.07, | ¹H NMR (500 MHz, DMSO-d₆) δ 7.50- |
| 49 | dimethyl-pyrimidine | 249.1 | 7.45 (m, 2H), 7.43-7.38 (m, 2H), 7.37- |
| | | | 7.32 (m, 1H), 5.43 (s, 2H), 2.49 (s, |
| | | | 3H), 2.14 (s, 3H). |
| Intermediate A - | 2-bromo-4-[(4- | 307.02, | ¹H NMR (400 MHz, CD₃OD) δ 8.03 (d, |
| 50 | methoxyphenyl)methoxy]- | 308.1 | J = 5.7 Hz, 1H), 7.37 (d, J = 8.7 Hz, |
| | 3-methyl-pyridine | | 2H), 7.09 (d, J = 5.7 Hz, 1H), 6.95 (d, |
| | | | J = 8.7 Hz, 2H), 5.14 (s, 2H), 3.80 (s, |
| | | | 3H), 2.28 (s, 3H). |
| Intermediate A - | 2-chloro-4-[(4- | 263.07, | ¹H NMR (400 MHz, DMSO-d₆) δ 8.06 |
| 51 | methoxyphenyl)methoxy]- | 264.16 | (s, 1H), 7.40 (d, J = 8.1 Hz, 2H), 7.18 |
| | 5-methyl-pyridine | | (s, 1H), 6.97 (d, J = 8.2 Hz, 2H), 5.16 |
| | | | (s, 2H), 3.76 (s, 3H), 2.07 (s, 3H) |

### Intermediate A-52

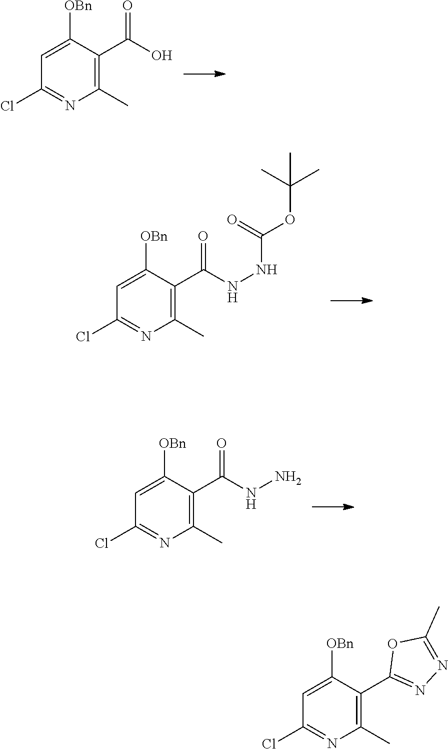 [SMILES: Cc1nc(Cl)cc(OCc2ccccc2)c1C(=O)O ; Cc1nc(Cl)cc(OCc2ccccc2)c1C(=O)NNC(=O)OC(C)(C)C ; Cc1nc(Cl)cc(OCc2ccccc2)c1C(=O)NN ; Cc1nnc(-c2c(OCc3ccccc3)cc(Cl)nc2C)o1]

### Step 1: tert-butyl N-[(4-benzyloxy-6-chloro-2-methyl-pyridine-3-carbonyl)amino]carbamate

To a stirring solution of 4-benzyloxy-6-chloro-2-methyl-pyridine-3-carboxylic acid (1.0 g, 2.9 mmol) and DMF (95 mg, 100 μL, 1.3 mmol) in dichloromethane (10 mL) at 0° C. was added oxalyl chloride (1.5 g, 1.0 mL, 11.5 mmol) dropwise. The mixture was allowed to warm to room temperature and stirred for 18 h. The reaction was concentrated, then redissolved in dichloromethane (10 mL) and added dropwise to a solution of Hunig's base (1.1 g, 1.5 mL, 8.6 mmol) and tert-butyl carbazate (420 mg, 3.18 mmol) in dichloromethane (10 mL) at 0° C. The reaction was warmed to room temperature and stirred for 5 h. Additional Hunig's base (1.1 g, 1.5 mL, 8.6 mmol) and tert-butyl carbazate (420 mg, 3.18 mmol) were added and the reaction mixture was stirred for 18 h. The reaction was diluted with water (100 mL) and extracted with ethyl acetate (100 mL). The organic layer was washed with 1 M HCl (100 mL), saturated aqueous sodium bicarbonate (100 mL), water (100 mL) and brine (100 mL). The organic phase was dried over magnesium sulfate, filtered and concentrated. Purification by silica gel chromatography (0-50% ethyl acetate/heptane) provided tert-butyl N-[(4-benzyloxy-6-chloro-2-methyl-pyridine-3-carbonyl)amino]carbamate (808 mg, 70%). ESI-MS m/z calc. 391.13, found 392.15 (M+1)⁺. ¹H NMR (400 MHz, CDCl₃) δ 7.66 (br s, 1H), 7.43-7.34 (m, 5H), 6.80 (s, 1H), 6.61 (br s, 1H), 5.15 (s, 2H), 2.61 (s, 3H), 1.50 (s, 9H).

### Step 2: 4-benzyloxy-6-chloro-2-methyl-pyridine-3-carbohydrazide

A solution of tert-butyl N-[(4-benzyloxy-6-chloro-2-methyl-pyridine-3-carbonyl)amino]carbamate (808 mg, 1.98 mmol) in HCl in dioxane (10 mL of 4 M, 40 mmol) was stirred at room temperature for 4 days. The mixture was diluted with diethyl ether, and the resulting solid filtered and dried to provide 4-benzyloxy-6-chloro-2-methyl-pyridine-3-carbohydrazide (dihydrochloride salt) (722 mg, 95%) as a pale yellow solid. ESI-MS m/z calc. 291.07, found 292.04 (M+1)⁺. ¹H NMR (400 MHz, DMSO-d₆) δ 11.72 (s, 1H), 7.44-7.33 (m, 5H), 7.27 (s, 1H), 5.29 (s, 2H), 2.37 (s, 3H).

### Step 3: 2-(4-benzyloxy-6-chloro-2-methyl-3-pyridyl)-5-methyl-1,3,4-oxadiazole

A solution of 4-benzyloxy-6-chloro-2-methyl-pyridine-3-carbohydrazide (100 mg, 0.27 mmol) in triethyl orthoacetate (89 mg, 1.0 mL, 5.5 mmol) was subjected to microwave irradiation at 120° C. for 1 h. The reaction mixture was cooled and partitioned between ethyl acetate (10 mL) and saturated aqueous sodium bicarbonate (10 mL). The organic layer was separated and washed with brine (10 mL), dried over magnesium sulfate, filtered and concentrated. Purification by reverse phase column chromatography (C18, 5-95% acetonitrile/water containing 0.1% formic acid) provided 2-(4-benzyloxy-6-chloro-2-methyl-3-pyridyl)-5-methyl-1,3,4-oxadiazole (45 mg, 44%). ESI-MS m/z calc. 315.07, found 316.04 (M+1)⁺. H NMR (400 MHz, CDCl₃) δ 7.39-7.30 (m, 5H), 6.86 (s, 1H), 5.15 (s, 2H), 2.58 (s, 3H), 2.52 (s, 3H).

### Intermediate A-53

### 2-(4-benzyloxy-6-chloro-2-methyl-3-pyridyl)-1,3,4-oxadiazole

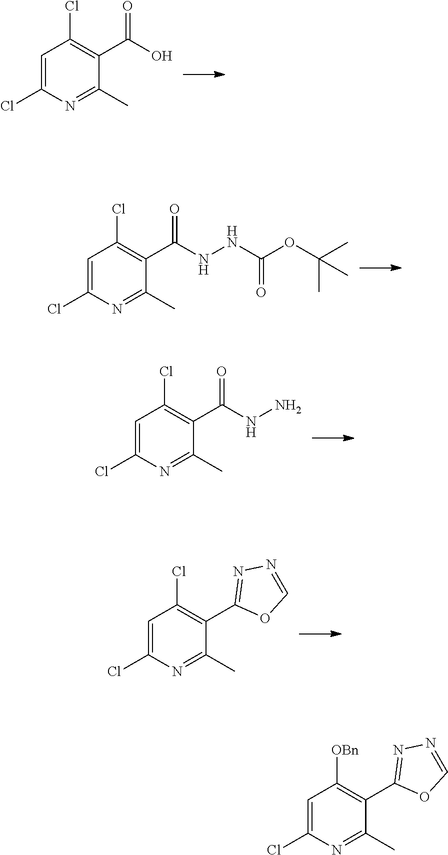 [SMILES: Cc1nc(Cl)cc(Cl)c1C(=O)O ; Cc1nc(Cl)cc(Cl)c1C(=O)NNC(=O)OC(C)(C)C ; Cc1nc(Cl)cc(Cl)c1C(=O)NN ; Cc1nc(Cl)cc(Cl)c1-c1nnco1 ; Cc1nc(Cl)cc(OCc2ccccc2)c1-c1nnco1]

### Step 1: tert-butyl N-[(4,6-dichloro-2-methyl-pyridine-3-carbonyl)amino]carbamate

A mixture of 4,6-dichloro-2-methyl-pyridine-3-carboxylic acid (7.0 g, 34 mmol) and SOCl₂ (24.5 g, 15.0 mL, 206 mmol) was refluxed for 2 h. The mixture was concentrated under vacuum, then dissolved in toluene and evaporated under reduced pressure to give 9 g of the corresponding acid chloride. A solution of the above acid chloride (9 g) in DCM (40 mL) was added slowly, at 0° C., to a mixture of tert-Butyl carbazate (4.5 g, 34 mmol) and TEA (10.9 g, 15.0 mL. 108 mmol) in DCM (80 mL). After 2 h of stirring, the reaction mixture was diluted with water (60 mL) and extracted with EtOAc (2×300 mL) and the organic layer was washed with sodium bicarbonate solution (100 mL) and brine (100 mL). The combined organic layers were dried over magnesium sulfate, filtered concentrated, Purification by silica gel column chromatography (16-20% ethyl acetate/hexane) provided tert-butyl N-[(4,6-dichloro-2-methyl-pyridine-3-carbonyl)amino]carbamate (7 g, 64%). ESI-MS m/z calc. 319.05, found 320.1 (M+1)⁺.

### Step 2: 4,6-dichloro-2-methyl-pyridine-3-carbohydrazide

A mixture of tert-butyl N-[(4,6-dichloro-2-methyl-pyridine-3-carbonyl)amino]carbamate (7.0 g, 22 mmol) and hydrochloric acid solution in dioxane (120 mL of 4.0 M, 480 mmol) was stirred at room temperature for 3 h. The solvent was evaporated to obtain 4,6-dichloro-2-methyl-pyridine-3-carbohydrazide (5 g, 98%). ESI-MS m/z calc. 218.99, found 220.07 (M+1)⁺.

### Step 3: 2-(4,6-dichloro-2-methyl-3-pyridyl)-1,3,4-oxadiazole

A mixture of 4,6-dichloro-2-methyl-pyridine-3-carbohydrazide (5.0 g, 27 mmol) and triethylorthoformate (22 g, 25 mL, 150 mmol) was heated at 140° C. for 16 h. The mixture was diluted with water (20 mL) and extracted with EtOAc (2×200 mL). The combined organic layers were washed with brine (80 mL), dried over magnesium sulfate, filtered concentrated. Purification by silica gel column chromatography (10-15% ethyl acetate/hexane) provided 2-(4,6-dichloro-2-methyl-3-pyridyl)-1,3,4-oxadiazole (3 g, 56%). ESI-MS m/z calc. 228.98, found 230.14 (M+1)⁺. ¹H NMR (400 MHz, DMSO-d₆) δ 9.60 (s, 1H), 7.99 (s, 1H), 2.43 (s, 3H).

### Step 4: 2-(4-benzyloxy-6-chloro-2-methyl-3-pyridyl)-1,3,4-oxadiazole

To a mixture of 2-(4,6-dichloro-2-methyl-3-pyridyl)-1,3,4-oxadiazole (3.0 g, 13 mmol) and benzyl alcohol (1.5 g, 1.4 mL, 13.5 mmol) in DMF (40 mL) was added potassium tert-butoxide (2.0 g, 18 mmol) portionwise at 0° C. The reaction mixture was gradually warmed to room temperature and stirred for 3 h. The mixture was diluted with water (50 mL) and extracted with ethyl acetate (2×200 mL). The combined organic layers were washed with brine (2×80 mL), dried over magnesium sulfate, filtered and concentrated. Purification by silica gel column chromatography (20-25% ethyl acetate/hexane) provided 2-(4-benzyloxy-6-chloro-2-methyl-3-pyridyl)-1,3,4-oxadiazole (1.7 g, 43%). ESI-MS m/z calc. 301.06, found 302.27 (M+1)⁺. ¹H NMR (400 MHz, DMSO-d₆) δ 9.48 (s, 1H), 7.44-7.28 (m, 6H), 5.34 (s, 2H), 2.37 (s, 3H).

### Intermediate A-54

### 3-(4-benzyloxy-6-chloro-2-methyl-3-pyridyl)-5-methyl-1,2,4-oxadiazole

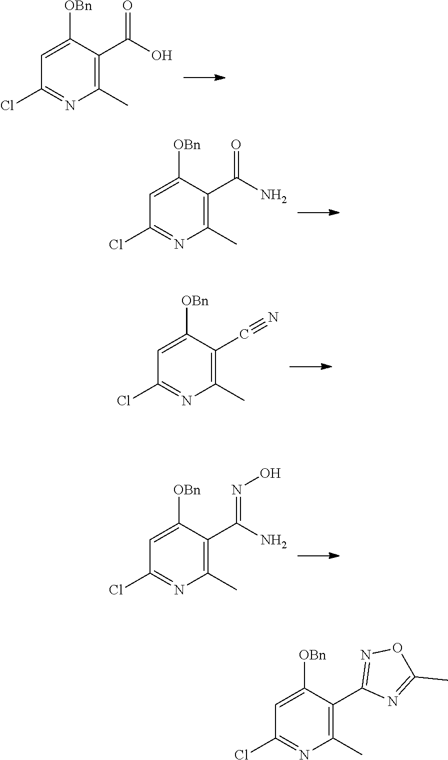 [SMILES: Cc1nc(Cl)cc(OCc2ccccc2)c1C(=O)O ; Cc1nc(Cl)cc(OCc2ccccc2)c1C(N)=O ; Cc1nc(Cl)cc(OCc2ccccc2)c1C#N ; Cc1nc(Cl)cc(OCc2ccccc2)c1/C(N)=N/O ; Cc1nc(-c2c(OCc3ccccc3)cc(Cl)nc2C)no1]

### Step 1: 4-benzyloxy-6-chloro-2-methyl-pyridine-3-carboxamide

To a stirring solution of 4-benzyloxy-6-chloro-2-methyl-pyridine-3-carboxylic acid (1.0 g, 2.9 mmol) and Hunig's base (1.1 g, 1.5 mL, 8.6 mmol) in DMF (5 mL) and dichloromethane (10 mL) at 0° C. was added ammonium chloride (460 mg, 0.301 mL, 8.60 mmol). The mixture was stirred for 30 min before addition of HATU (1.3 g, 3.4 mmol). The reaction was allowed to warm to room temperature and stirred for 20 h. Additional ammonium chloride (230 mg, 0.150 mL, 4.30 mmol), Hunig's base (560 mg, 0.75 mL, 4.3 mmol) and HATU (650 mg, 1.71 mmol) were added and the reaction stirred for 4 days. The mixture was diluted with water (100 mL) and extracted with ethyl acetate (100 mL). The organic phase was dried over magnesium sulfate and concentrated. Purification by silica gel chromatography (0-100% ethyl acetate/heptane) provided 4-benzyloxy-6-chloro-2-methyl-pyridine-3-carboxamide (743 mg, 89%) as a white solid. ¹H NMR (400 MHz, CDCl₃) δ 7.44-7.35 (m, 5H), 6.83 (s, 1H), 5.88 (d, J=18.8 Hz, 2H), 5.16 (s, 2H), 2.59 (s, 3H). ESI-MS m/z calc. 276.07, found 277.05 (M+1)⁺.

### Step 2: 4-benzyloxy-6-chloro-2-methyl-pyridine-3-carbonitrile

To a stirring mixture of 4-benzyloxy-6-chloro-2-methyl-pyridine-3-carboxamide (640 mg, 2.20 mmol), triethylamine (1.0 mL, 7.2 mmol) and DMF (20 μL, 0.26 mmol) in DCM (6 mL) at 0° C. was added oxalyl chloride (0.39 mL, 4.5 mmol) dropwise. The mixture was allowed to warm to room temperature and stirred for 20 h. The mixture was partitioned between DCM (50 mL) and water (50 mL). The organic phase was separated, washed with 1 M aqueous HCl (50 mL) and brine (50 mL), dried over magnesium sulfate and concentrated to provide 4-benzyloxy-6-chloro-2-methyl-pyridine-3-carbonitrile (1.1 g, 97%). ¹H NMR (400 MHz, CDCl₃) δ 7.46-7.37 (m, 5H), 6.84 (s, 1H), 5.25 (s, 2H), 2.70 (s, 3H). ESI-MS m/z calc. 258.06, found 259.08 (M+1)⁺.

### Step 3: 4-benzyloxy-6-chloro-N-hydroxy-2-methyl-pyridine-3-carboxamidine

A mixture of 4-benzyloxy-6-chloro-2-methyl-pyridine-3-carbonitrile (440 mg, 1.70 mmol), sodium carbonate (450 mg, 4.25 mmol) and hydroxylamine hydrochloride (590 mg, 8.49 mmol) in methanol (10 mL) and water (1 mL) was stirred at 65° C. for 3 days. The mixture was cooled and partitioned between ethyl acetate (100 mL) and water (100 mL). The organic phase was separated, washed with brine (100 mL), dried over magnesium sulfate and concentrated to provide 4-benzyloxy-6-chloro-N-hydroxy-2-methyl-pyridine-3-carboxamidine (371 mg, 58%). ESI-MS m/z calc. 291.08, found 292.05 (M+1)⁺.

### Step 4: 3-(4-benzyloxy-6-chloro-2-methyl-3-pyridyl)-5-methyl-1,2,4-oxadiazole

To a solution of 4-benzyloxy-6-chloro-N-hydroxy-2-methyl-pyridine-3-carboxamidine (371 mg, 0.983 mmol) and triethylamine (0.35 mL, 2.5 mmol) in acetone (5 mL) at 0° C. was added acetyl chloride (99 mg, 90 μL, 1.3 mmol). The reaction was allowed to warm to room temperature and stirred for 20 h. The mixture was cooled to 0° C. and additional triethylamine (0.35 mL, 2.5 mmol) and acetyl chloride (99 mg, 90 μL, 1.3 mmol) added. The mixture was allowed to warm to room temperature and stirred for 20 h. The mixture was partitioned between ethyl acetate (20 mL) and water (20 mL). The organic phase was separated, washed with brine (20 mL), dried over magnesium sulfate and concentrated. The residue was dissolved in DMSO (3 mL) and potassium hydroxide (70 mg, 1.25 mmol) added. The mixture was stirred at room temperature for 2 h. The reaction mixture was directly purified by reverse phase chromatography (C18, 5-95% acetonitrile in water, 0.1% v/v ammonia) to provide 3-(4-benzyloxy-6-chloro-2-methyl-3-pyridyl)-5-methyl-1,2,4-oxadiazole (55 mg, 17%) as a white solid. ¹H NMR (400 MHz, CDCl₃) δ 7.39-7.30 (m, 5H), 6.83 (s, 1H), 5.15 (s, 2H), 2.69 (s, 3H), 2.43 (s, 3H). ESI-MS m/z calc. 315.08, found 316.04 (M+1)⁺.

### Intermediate A-55

### 5-(4-benzyloxy-6-chloro-2-methyl-3-pyridyl)-3-methyl-1,2,4-oxadiazole

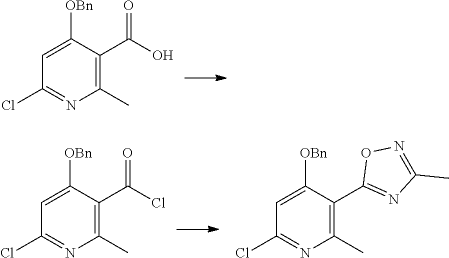 [SMILES: Cc1nc(Cl)cc(OCc2ccccc2)c1C(=O)O ; Cc1nc(Cl)cc(OCc2ccccc2)c1C(=O)Cl ; Cc1noc(-c2c(OCc3ccccc3)cc(Cl)nc2C)n1]

### Step 1: 4-benzyloxy-6-chloro-2-methyl-pyridine-3-carbonyl chloride

To a solution of 4-benzyloxy-6-chloro-2-methyl-pyridine-3-carboxylic acid (200 mg, 0.67 mmol) in DCM (2 mL) at 0° C. was added oxalyl chloride (102 mg. 0.07 mL, 0.8 mmol) and DMF (11 mg, 12 μL, 0.15 mmol) and the mixture was warmed to room temperature for 4 h. The reaction mixture was concentrated under reduced pressure and co-evaporated with DCM (3×10 mL) to provide 4-benzyloxy-6-chloro-2-methyl-pyridine-3-carbonyl chloride (200 mg, 100%).

### Step 2: 5-(4-benzyloxy-6-chloro-2-methyl-3-pyridyl)-3-methyl-1,2,4-oxadiazole

A mixture of 4-benzyloxy-6-chloro-2-methyl-pyridine-3-carbonyl chloride (2.22 g, 6.37 mmol), potassium carbonate (2.65 g, 19.17 mmol) and acetamide oxime (710 mg, 9.58 mmol) in DMF (20 mL) was stirred at room temperature for 3 days. Additional acetamide oxime (350 mg, 4.72 mmol) and potassium carbonate (1.3 g, 9.4 mmol) were added and the reaction was stirred at 100° C. for 3 h. The mixture was cooled to room temperature and partitioned between ethyl acetate (100 mL) and water (100 mL). The layers were separated and the organic layer was washed with water (100 mL) and brine (100 mL), dried over magnesium sulfate, filtered and concentrated. Purification by reverse phase column chromatography (C18, 5-95% acetonitrile in water, both containing 0.1% formic acid) provided 5-(4-benzyloxy-6-chloro-2-methyl-3-pyridyl)-3-methyl-1,2,4-oxadiazole (383 mg, 18%). ESI-MS m/z calc. 315.07, found 316.06 (M+1)⁺. H NMR (400 MHz, CDCl₃) δ 7.41-7.32 (m, 5H), 6.87 (s, 1H), 5.19 (s, 2H), 2.52 (s, 3H), 2.51 (s, 3H).

### Intermediate A-56

### 4-benzyloxy-6-chloro-2-methyl-3-(2-methyltetrazol-5-yl)pyridine

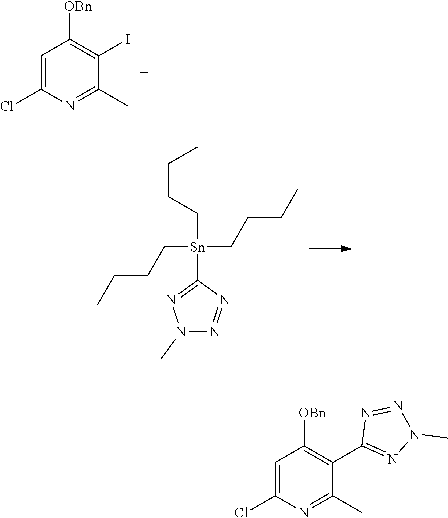 [SMILES: Cc1nc(Cl)cc(OCc2ccccc2)c1I ; CCC[CH2][Sn]([CH2]CCC)([CH2]CCC)[c]1nnn(C)n1 ; Cc1nc(Cl)cc(OCc2ccccc2)c1-c1nnn(C)n1]

A microwave vial was charged with 4-benzyloxy-6-chloro-3-iodo-2-methyl-pyridine (206 mg, 0.572 mmol), tributyl-(2-methyltetrazol-5-yl)stannane (273 mg, 0.732 mmol), CuI (15 mg, 0.078 mmol) and Pd(PPh₃)₂Cl₂ (82.6 mg, 0.118 mmol). The mixture was dissolved in toluene (3 mL), degassed for 5 min and placed under a nitrogen atmosphere. The tube was sealed and stirred at 110° C. for 20 h. The mixture was diluted with ethyl acetate, washed with a saturated aqueous ammonium chloride and brine. The organic layer was dried over magnesium sulfate, filtered, and concentrated. Purification by silica gel chromatography (0-70% ethyl acetate/hexanes over 20 min) provided 4-benzyloxy-6-chloro-2-methyl-3-(2-methyltetrazol-5-yl)pyridine (172.5 mg, 91%). ¹H NMR (400 MHz, DMSO-d₆) δ 7.40-7.25 (m, 6H), 5.29 (s, 2H), 4.46 (s, 3H), 2.23 (s, 3H). ESI-MS m/z calc. 315.09, found 316.2 (M+1)⁺.

Intermediates in Table 2 were prepared from 4-benzyloxy-6-chloro-3-iodo-2-methyl-pyridine and the appropriate stannane using a procedure analogous to that described for Intermediate A-56 (4-benzyloxy-6-chloro-2-methyl-3-(2-methyltetrazol-5-yl)pyridine).

TABLE 2

| | | MW & | |
|---|---|---|---|
| | Compound | Found | |
| Intermediate | Name | [M + H]⁺ | NMR (shifts in ppm) |
| Intermediate A - | 2-(4-benzyloxy-6-chloro-2- | 341.09; | ¹H NMR (400 MHz, CD₃OD) δ 8.24 (s, |
| 57 | methyl-3-pyridyl)-3- | 342.38 | 2H), 7.36-7.24 (m, 3H), 7.24-7.17 (m, |
| | methoxy-pyrazine | | 2H), 7.14 (s, 1H), 5.15 (s, 2H), 3.91 (s, |
| | | | 3H), 2.18 (s, 3H) |
| Intermediate A - | 4-benzyloxy-6-chloro-3- | 327.11; | ¹H NMR (400 MHz, CD₃OD) δ 7.38- |
| 58 | (1,2-dimethylimidazol-4- | 328.4 | 7.25 (m, 5H), 7.03 (s, 2H), 5.15 (s, 2H), |
| | yl)-2-methyl-pyridine | | 3.64 (s, 3H), 2.40 (s, 3H), 2.36 (s, 3H) |
| Intermediate A - | 4-(4-benzyloxy-6-chloro-2- | 311.08; | |
| 59 | methy1-3- | 312.0 | |
| | pyridyl)pyrimidine | | |
| Intermediate A - | 2-(4-benzyloxy-6-chloro-2- | 314.08; | ¹H NMR (400 MHz, CD₃OD) δ 7.81- |
| 60 | methyl-3-pyridyl)-4-methyl- | 315.3 | 7.76 (m, 1H), 7.39-7.27 (m, 5H), 7.18 |
| | oxazole | | (s, 1H), 5.23 (s, 2H), 2.35 (s, 3H), 2.24 |
| | | | (d, J = 1.2 Hz, 3H). |
| Intermediate A - | 4-benzyloxy-6-chloro-2- | 314.09; | ¹H NMR (400 MHz, DMSO-d₆) δ 7.86 |
| 61 | methyl-3-(2-methyltriazol- | 315.3 | (s, 1H), 7.43-7.30 (m, 5H), 7.26 (s, |
| | 4-y1)pyridine | | 1H), 5.25 (s, 2H), 4.20 (s, 3H), 2.37 (s, |
| | | | 3H) |
| Intermediate A - | 2-(4-benzyloxy-6-chloro-2- | 300.07; | ¹H NMR (400 MHz, CD₃OD) δ 8.09 |
| 62 | methyl-3-pyridyl)oxazole | 301.2 | (d, J = 0.9 Hz, 1H), 7.38 (d, J = 0.9 Hz, |
| | | | IH), 7.37-7.27 (m, 5H), 7.20 (s, 1H), |
| | | | 5.23 (s, 2H), 2.35 (s, 3H) |
| Intermediate A - | 2-(4-benzyloxy-6-chloro-2- | 314.08; | ¹H NMR (400 MHz, CD₃OD) δ 7.40- |
| 63 | methyl-3-pyridyl)-5-methyl- | 315.1 | 7.26 (m, 5H), 7.18 (s, 1H), 7.01-6.96 |
| | oxazole | | (m, 1H), 5.23 (s, 2H), 2.38 (d, J = 1.1 |
| | | | Hz, 3H), 2.37 (s, 3H) |
| Intermediate A - | 4-benzyloxy-6-chloro-3-(6- | 340.10; | ¹H NMR (400 MHz, CD₃OD) δ 7.74 |
| 64 | methoxy-2-pyridyl)-2- | 341.3 | (dd, J = 8.4, 7.3 Hz, 1H), 7.35-7.20 (m, |
| | methyl-pyridine | | 5H), 7.12 (s, 1H), 6.98 (dd, J = 7.3, 0.8 |
| | | | Hz, 1H), 6.79 (dd, J = 8.4, 0.8 Hz, 1H), |
| | | | 5.17 (s, 2H), 3.89 (s, 3H), 2.26 (s, 3H) |

### Intermediate A-65

### 4-benzyloxy-6-chloro-2-methyl-3-(1-methylpyrazol-4-yl)pyridine

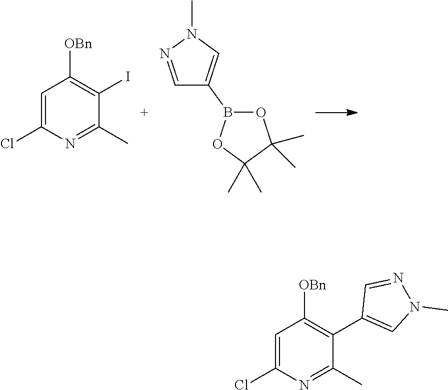

A mixture of 4-benzyloxy-6-chloro-3-iodo-2-methyl-pyridine (107 mg, 0.298 mmol), 1-methyl-4-(4,4,5,5-tetramethyl-1,3,2-dioxaborolan-2-yl)pyrazole (76 mg, 0.37 mmol), PdCl₂(dtbpf) (32.5 mg, 0.0499 mmol) and potassium phosphate (275 mg, 1.29 mmol) in dioxane (2 mL) and water (500 L) in a microwave vial was nitrogen degassed for 5 min then stirred at 45° C. for 1 h. The mixture was diluted with ethyl acetate and washed with saturated aqueous ammonium chloride and brine. The organic layer was dried over magnesium sulfate, filtered, and concentrated. Purification by silica gel chromatography (0-75% ethyl acetate/hexanes over 20 min) provided 4-benzyloxy-6-chloro-2-methyl-3-(1-methylpyrazol-4-yl)pyridine (67 mg, 72%). ¹H NMR (400 MHz, CD₃OD) δ 7.73 (s, 1H), 7.56 (s, 1H), 7.40-7.25 (m, 5H), 7.08 (s, 1H), 5.18 (s, 2H), 3.91 (s, 3H), 2.43 (s, 3H). ESI-MS m/z calc. 313.10, found 314.26 (M+1)⁺.

Intermediates in Table 3 were prepared from 4-benzyloxy-6-chloro-3-iodo-2-methyl-pyridine or ethyl 4-benzyloxy-2-chloro-5-iodo-6-methyl-pyridine-3-carboxylate and the appropriate boronic acid or boronic ester using a procedure analogous to that described for Intermediate A-56 (4-benzyloxy-6-chloro-2-methyl-3-(1-methylpyrazol-4-yl)pyridine).

TABLE 3

| | | MW & | |
|---|---|---|---|
| | Compound | Found | |
| Intermediate | Name | [M + H]⁺ | NMR (shifts in ppm) |
| Intermediate A - | tert-butyl 5-(4-benzyloxy-6- | 299.08; | ¹H NMR (400 MHz, CD₃OD) δ 7.80- |
| 66 | chloro-2-methyl-3- | 300.3 | 7.57 (m, 1H), 7.36-7.24 (m, 5H), 7.10 |
| 11433 | pyridyl)pyrazole-1- | | (br s, 1H), 6.39 (d, J = 2.3 Hz, 1H), |
| | carboxylate | | 5.17 (s, 2H), 2.32 (s, 3H) |
| Intermediate A - | 5-(4-benzyloxy-6-chloro-2- | 300.07; | ¹H NMR (400 MHz, CD₃OD) δ 8.33 (s, |
| 67 | methyl-3-pyridyl)oxazole | 301.2 | 1H), 7.42-7.34 (m, 5H), 7.34 (s, 1H), |
| | | | 7.19 (s, 1H), 5.25 (s, 2H), 2.50 (s, 3H) |
| Intermediate A - | 4-benzyloxy-3-(2- | 416.1; | ¹H NMR (400 MHz, CD₃OD) & 8.20 |
| 68 | benzyloxy-3-pyridyl)-6- | 417.4 | (dd, J = 5.0, 1.9 Hz, 1H), 7.57 (dd, J = |
| | chloro-2-methyl-pyridine | | 7.3, 1.9 Hz, 1H), 7.27-7.17 (m, 8H), |
| | | | 7.17-7.10 (m, 2H), 7.09-7.04 (m, 2H), |
| | | | 5.37 (d, J = 12.7 Hz, 1H), 5.32 (d, J = |
| | | | 12.6 Hz, 1H), 5.13 (d, J = 12.4 Hz, 1H), |
| | | | 5.06 (d, J = 12.4 Hz, 1H), 2.17 (s, 3H). |
| Intermediate A - | 2-benzyloxy-5-(4- | 417.12; | ¹H NMR (400 MHz, CD₃OD) δ 8.53 (s, |
| 69 | benzyloxy-6-chloro-2- | 418.4 | 2H), 7.51-7.44 (m, 2H), 7.41-7.22 (m, |
| | methyl-3- | | 8H), 7.18 (s, 1H), 5.49 (s, 2H), 5.17 (s, |
| | pyridyl)pyrimidine | | 2H), 2.32 (s, 3H) |
| Intermediate A - | 4-benzyloxy-3-(2- | 416.13; | ¹H NMR (400 MHz, CD₃OD) & 8.20 |
| 70 | benzyloxy-4-pyridyl)-6- | 417.5 | (dd, J = 5.2, 0.8 Hz, 1H), 7.47-7.40 (m, |
| | chloro-2-methyl-pyridine | | 2H), 7.39-7.20 (m, 8H), 7.13 (s, 1H), |
| | | | 6.88 (dd, J = 5.3, 1.4 Hz, 1H), 6.79 (dd, |
| | | | J = 1.3, 0.8 Hz, 1H), 5.38 (s, 2H), 5.16 |
| | | | (s, 2H), 2.25 (s, 3H) |
| Intermediate A - | 6-(4-benzyloxy-6-chloro-2- | 335.08; | ¹H NMR (400 MHz, CDCl₃) δ 7.92 (t, |
| 71 | methyl-3-pyridyl)pyridine- | 336.2 | J = 7.8 Hz, 1H), 7.72 (dd, J = 7.7, 1.1 |
| | 2-carbonitrilc | | Hz, 1H), 7.57 (dd, J = 8.0, 1.1 Hz, 1H), |
| | | | 7.41-7.29 (m, 3H), 7.22 (dd, J = 7.7, |
| | | | 1.8 Hz, 2H), 6.89 (s, 1H), 5.10 (s, 2H), |
| | | | 2.35 (s, 3H) |
| Intermediate A - | 4-benzyloxy-3-(6- | 416.13; | ¹H NMR (400 MHz, CDCl₃) δ 8.07 (s, |
| 72 | benzyloxy-3-pyridyl)-6- | 417.4 | 1H), 7.53-7.46 (m, 3H), 7.44-7.37 (m, |
| | chloro-2-methyl-pyridine | | 2H), 7.37-7.29 (m, 4H), 7.24-7.17 (m, |
| | | | 2H), 6.90 (d, J = 8.4 Hz, 1H), 6.83 (s, |
| | | | 1H), 5.44 (s, 2H), 5.09 (s, 2H), 2.36 (s, |
| | | | 3H) |
| Intermediate A - | 5-(4-benzyloxy-6-chloro-2- | 314.08; | ¹H NMR (400 MHz, CD₃OD) δ 7.39- |
| 73 | methyl-3-pyridyl)-3-methyl- | 315.4 | 7.30 (m, 5H), 7.19 (s, 1H), 6.49 (s, 1H), |
| | isoxazole | | 5.24 (s, 2H), 2.43 (s, 3H), 2.32 (s, 3H) |
| Intermediate A- | ethyl 4-benzyloxy-5-(2- | 488.15; | ¹H NMR (400 MHz, DMSO-d₆) δ 8.31 |
| 74 | benzyloxy-3-pyridyl)-2- | 489.6 | (dd, J = 5.0, 1.9 Hz, 1H), 7.77 (dd, J = |
| | chloro-6-methyl-pyridine-3- | | 7.4, 1.9 Hz, 1H), 7.33-7.20 (m, 8H), |
| | carboxylate | | 7.15 (dd, J = 7.3, 5.0 Hz, 1H), 6.91- |
| | | | 6.84 (m, 2H), 5.43-5.30 (m, 2H), 4.66- |
| | | | 4.55 (m, 2H), 4.38-4.26 (m, 2H), 2.19 |
| | | | (s, 3H), 1.24 (t, J = 7.1 Hz, 3H) |
| Intermediate A - | ethyl 4-benzyloxy-5-(6- | 488.15; | |
| 75 | benzyloxy-2-pyridyl)-2- | 489.77 | |
| | chloro-6-methyl-pyridine-3- | | |
| | carboxylate | | |
| Intermediate A - | ethyl 4-benzyloxy-2-chloro- | 345.11; | ¹H NMR (400 MHz, DMSO-d₆) δ 7.46- |
| 76 | 5-cyclopropyl-6-methyl- | 347.41 | 7.33 (m, 5H), 5.18 (s, 2H), 4.22 (q, J = |
| | pyridine-3-carboxylate | | 7.1 Hz, 2H), 2.57 (s, 3H), 1.88 (td, J = |
| | | | 8.3, 4.2 Hz, 1H), 1.17 (t, J = 7.1 Hz, |
| | | | 3H), 1.13-1.04 (m, 2H), 0.72-0.58 (m, |
| | | | 2H) |

### Intermediate A-77

### 5-(4-benzyloxy-6-chloro-2-methyl-3-pyridyl)oxazolidin-2-one

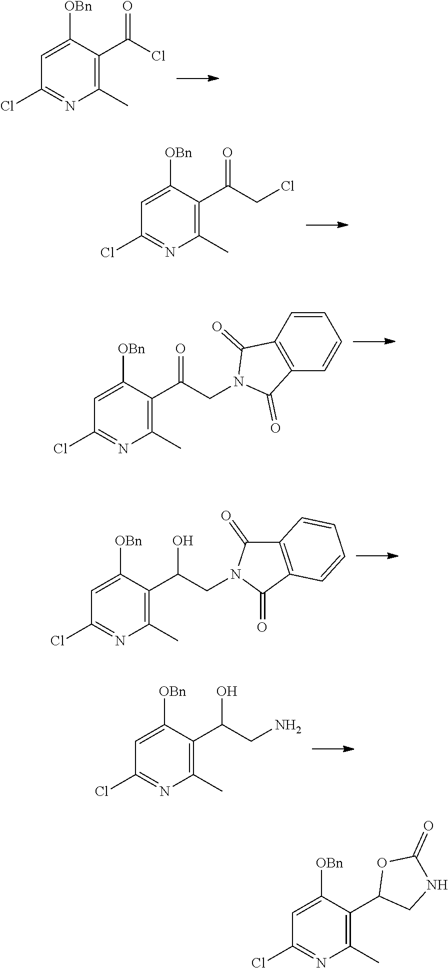 [SMILES: Cc1nc(Cl)cc(OCc2ccccc2)c1C(=O)Cl ; Cc1nc(Cl)cc(OCc2ccccc2)c1C(=O)CCl ; Cc1nc(Cl)cc(OCc2ccccc2)c1C(=O)CN1C(=O)c2ccccc2C1=O ; Cc1nc(Cl)cc(OCc2ccccc2)c1C(O)CN1C(=O)c2ccccc2C1=O ; Cc1nc(Cl)cc(OCc2ccccc2)c1C(O)CN ; Cc1nc(Cl)cc(OCc2ccccc2)c1C1CNC(=O)O1]

### Step 1: 1-4-benzyloxy-6-chloro-2-methyl-3-pyridyl-2-chloro-ethanone

A solution of 4-benzyloxy-6-chloro-2-methyl-pyridine-3-carbonyl chloride (10.9 g, 35.0 mmol) in THF (200 mL) at 0° C. was treated dropwise with diazomethyl(trimethyl)silane (35 mL of 2.0 M in hexanes, 70 mmol). The mixture was allowed to warm to room temperature and stirred overnight. Additional (trimethylsilyl)diazomethane) (17.5 mL of 2.0 M, 35 mmol) was added and the mixture stirred at room temperature for 72 h. The mixture was cooled to 0° C. and hydrochloric acid in 1,4-dioxane (17.5 mL of 4.0 M, 70 mmol) was added dropwise. The mixture was stirred at 0° C. for 30 min then at room temperature for 1 h. The mixture was added into saturated aqueous sodium bicarbonate solution (600 mL) and extracted with ethyl acetate (1 L). The organic phase was washed with brine (250 mL), dried over magnesium sulfate, filtered and concentrated. Purification by silica gel chromatography (0-20% EtOAc/heptanes) provided 1-(4-benzyloxy-6-chloro-2-methyl-3-pyridyl)-2-chloro-ethanone (6.78 g, 60%). ¹H NMR (400 MHz, CDCl₃) δ 7.44-7.34 (m, 5H), 6.84 (s, 1H), 5.14 (s, 2H), 4.37 (s, 2H), 2.45 (s, 3H). ESI-MS m/z calc. 309.03, found 309.97 (M+1)⁺.

### Step 2: 2-[2-(4-benzyloxy-6-chloro-2-methyl-3-pyridyl)-2-oxo-ethyl]isoindoline-1,3-dione

A mixture of 1-(4-benzyloxy-6-chloro-2-methyl-3-pyridyl)-2-chloro-ethanone (9.18 g, 28.1 mmol) and phthalimide potassium salt (5.75 g, 31.0 mmol) in DMF (200 mL) was stirred for 48 h at room temperature. The mixture was partitioned between ethyl acetate (800 mL) and water (800 mL) and the layers separated. The organic phase was washed with water (2×600 mL) and brine, dried over magnesium sulphate, filtered and concentrated. The residue was triturated in diethyl ether (2×100 mL). The resulting solid was isolated by filtration and dried under reduced pressure at 50° C. to provide 2-[2-(4-benzyloxy-6-chloro-2-methyl-3-pyridyl)-2-oxo-ethyl]isoindoline-1,3-dione (9.77 g, 81%). ¹H NMR (400 MHz, CDCl₃) δ 7.92-7.83 (m, 2H), 7.80-7.69 (m, 2H), 7.47-7.36 (m, 5H), 6.85 (s, 1H), 5.23 (s, 2H), 4.84 (s, 2H), 2.50 (s, 3H). ESI-MS m/z calc. 420.09, found 421.12 (M+1)⁺.

### Step 3: 2-[2-(4-benzyloxy-6-chloro-2-methyl-3-pyridyl)-2-hydroxy-ethyl]isoindoline-1,3-dione

To a solution of 2-[2-(4-benzyloxy-6-chloro-2-methyl-3-pyridyl)-2-oxo-ethyl]isoindoline-1,3-dione (50 mg, 0.11 mmol) in acetic acid (1.5 mL) was added sodium cyanoborohydride (14 mg, 0.22 mmol). The mixture was stirred at room temperature for 2 h then treated with additional sodium cyanoborohydride (14 mg, 0.22 mmol) and stirred for 16 h. A further batch of sodium cyanoborohydride (50 mg, 0.80 mmol) was added and the mixture stirred for 24 h. Additional sodium cyanoborohydride (100 mg, 1.59 mmol) was added and the mixture stirred over 48 h. The reaction mixture was diluted with saturated aqueous sodium bicarbonate solution (100 mL) and extracted with ethyl acetate. The organic phase was washed with brine, dried over magnesium sulfate, filtered and concentrated. Purification by reverse phase chromatography (C18, 20-80% acetonitrile/water each with 0.1% formic acid) provided 2-[2-(4-benzyloxy-6-chloro-2-methyl-3-pyridyl)-2-hydroxy-ethyl]isoindoline-1,3-dione (30 mg, 61%). ¹H NMR (400 MHz, CDCl₃) δ 7.83-7.80 (m, 2H), 7.74-7.69 (m, 2H), 7.54-7.40 (m, 5H), 6.86 (s, 1H), 5.35-5.14 (m, 3H), 4.28-4.15 (m, 1H), 3.91 (dd, J=13.7, 5.3 Hz, 1H), 3.35 (d, J=11.4 Hz, 1H), 2.54 (s, 3H). ESI-MS m/z calc. 422.10, found 423.13 (M+1)⁺.

### Step 4: 2-amino-1-(4-benzyloxy-6-chloro-2-methyl-3-pyridyl)ethanol

To a solution of 2-[2-(4-benzyloxy-6-chloro-2-methyl-3-pyridyl)-2-hydroxy-ethyl]isoindoline-1,3-dione (2.47 g, 5.45 mmol) in ethanol (100 mL) was added hydrazine hydrate (2.73 g, 2.65 mL, 54.5 mmol). The mixture was stirred at room temperature for 48 h. The precipitate was removed by filtration and the residue concentrated under reduced pressure. Purification by reverse phase chromatography (C18, 20-70% acetonitrile/water each with 0.1% ammonia) provided 2-amino-1-(4-benzyloxy-6-chloro-2-methyl-3-pyridyl)ethanol (1.65 g, 87%). ¹H NMR (400 MHz, CD₃OD) δ 7.51-7.31 (m, 5H), 7.01 (s, 1H), 5.22-5.17 (m, 2H), 5.16-5.11 (m, 1H), 3.13-3.00 (m, 1H), 2.80-2.70 (m, 1H), 2.56 (s, 3H). ESI-MS m/z calc. 292.10, found 293.05 (M+1)⁺.

### Step 5: 5-(4-benzyloxy-6-chloro-2-methyl-3-pyridyl)oxazolidine-2-one

To a solution of 2-amino-1-(4-benzyloxy-6-chloro-2-methyl-3-pyridyl)ethanol (1.55 g, 4.43 mmol) in DCM (25 mL) was added CDI (755 mg, 4.66 mmol) and the mixture stirred at room temperature overnight. The mixture was partitioned between DCM (250 mL) and water (100 mL) and the phases separated. The organic phase was washed with brine, dried over magnesium sulfate, filtered and concentrated. Purification by silica gel chromatography (0-3% methanol/DCM) provided 5-(4-benzyloxy-6-chloro-2-methyl-3-pyridyl) oxazolidine-2-one (762 mg, 53%). ¹H NMR (400 MHz, DMSO-d₆) δ 7.61 (s, 1H), 7.44-7.42 (m, 2H), 7.38-7.28 (m, 3H), 7.18 (s, 1H), 5.89 (t, J=9.2 Hz, 1H), 5.31-5.21 (m, 2H), 3.72 (t, J=8.9 Hz, 1H), 3.48-3.43 (m, 1H), 2.41 (s, 3H). ESI-MS m/z calc. 318.08, found 319.03 (M+1)⁺.

### Intermediate A-78

### 4-(4-benzyloxy-6-chloro-2-methyl-3-pyridyl)oxazolidine-2-one

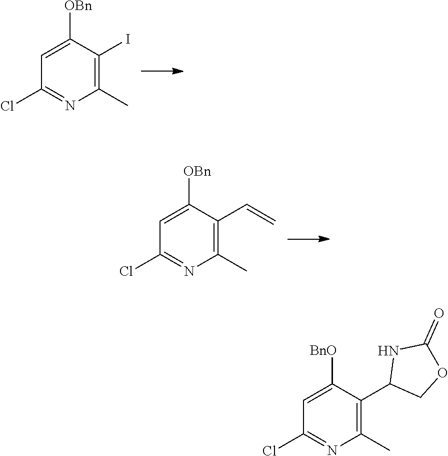 [SMILES: Cc1nc(Cl)cc(OCc2ccccc2)c1I ; C=Cc1c(OCc2ccccc2)cc(Cl)nc1C ; Cc1nc(Cl)cc(OCc2ccccc2)c1C1COC(=O)N1]

### Step 1: 4-benzyloxy-6-chloro-2-methyl-3-vinyl-pyridine

A solution of 4-benzyloxy-6-chloro-3-iodo-2-methyl-pyridine (7.13 g, 19.4 mmol), vinyl boronic acid pinacol ester (3.6 g, 4.0 mL, 24 mmol), Pd(dppf)Cl₂ (1.43 g, 1.95 mmol) and aqueous sodium carbonate (30 mL of 2.0 M, 60 mmol) in dioxane (60 mL) was stirred at 70° C. under argon for 20 h. The mixture was cooled and partitioned between ethyl acetate (200 mL) and water (200 mL). The aqueous layer extracted with additional ethyl acetate (100 mL), and the combined organic extracts washed with brine, dried over magnesium sulfate and concentrated. Purification by silica gel chromatography (0-5% ethyl acetate/heptane) provided 4-benzyloxy-6-chloro-2-methyl-3-vinyl-pyridine (4.79 g, 75%). ¹H NMR (400 MHz, CDCl₃) δ 7.48-7.35 (m, 5H), 6.78 (s, 1H), 6.72-6.62 (m, 1H), 5.75 (dd, J=17.9, 1.8 Hz, 1H), 5.58 (dd, J=11.4, 1.8 Hz, 1H), 5.13 (s, 2H), 2.55 (s, 3H). ESI-MS m/z calc. 259.08, found 260.03 (M+1)⁺.

### Step 2: 4-(4-benzyloxy-6-chloro-2-methyl-3-pyridyl)oxazolidin-2-one

To a solution of diphenyl diselenide (120 mg, 0.385 mmol), ammonium persulfate (105 mg, 0.460 mmol) and triflic acid (34 μL, 0.38 mmol) in dioxane (1 mL) was added a solution of 4-benzyloxy-6-chloro-2-methyl-3-vinyl-pyridine (100 mg, 0.381 mmol) and ethyl carbamate (102 mg, 1.15 mmol) in dioxane (1 mL). The mixture was stirred at 100° C. for 18 h. Additional diphenyl diselenide (120 mg, 0.385 mmol), ammonium persulfate (105 mg, 0.460 mmol) and triflic acid (34 μL, 0.38 mmol) were combined in dioxane (1 mL) and added to the reaction mixture, which was stirred for an additional 18 h at 100° C. Ammonium persulfate (105 mg, 0.4601 mmol) was added and the reaction stirred for 3 h. The mixture was cooled to room temperature and partitioned between ethyl acetate (20 mL) and water (20 mL). The organic phase was separated, washed with brine, dried over magnesium sulfate, filtered and concentrated. Purification by reverse phase chromatography (5-80% acetonitrile in water, 0.1% v/v formic acid) provided 4-(4-benzyloxy-6-chloro-2-methyl-3-pyridyl)oxazolidin-2-one (20 mg, 14%). ¹H NMR (400 MHz, CDCl₃) δ 7.44-7.36 (m, 5H), 6.84 (s, 1H), 5.37 (dd, J=10.1, 6.0 Hz, 1H), 5.18 (s, 2H), 4.69 (dd, J=9.8, 8.5 Hz, 1H), 4.37 (dd, J=8.7, 6.0 Hz, 1H), 2.56 (s, 3H). NH not observed. ESI-MS m/z calc. 318.08, found 319.07 (M+1)⁺.

### Intermediate A-79

### 4-benzyloxy-2-chloro-3-(2,5-dihydrofuran-3-yl)-5,6-dimethyl-pyridine

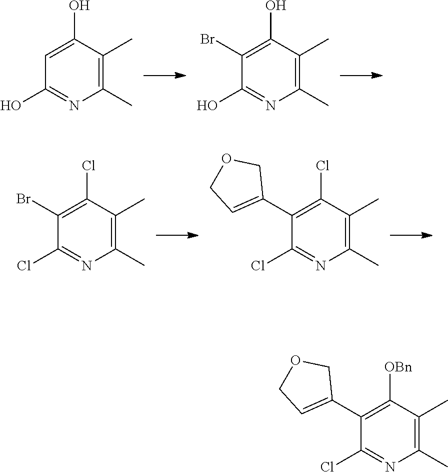

### Step 1: 3-bromo-5,6-dimethyl-pyridine-2,4-diol

To a solution of 5,6-dimethylpyridine-2,4-diol (15.0 g, 108 mmol) in dichloromethane (1 L) at 0° C. was added a solution of bromine (18.6 g, 6.0 mL, 116 mmol) in dichloromethane (50 mL) and the mixture stirred for 1 h at room temperature. The resulting yellow precipitate was filtered and washed with dichloromethane. The solid was combined with sodium bicarbonate (15 g, 179 mmol) in THF (1 L) and heated at 70° C. for 15 min. The mixture was filtered and concentrated to afford 3-bromo-5,6-dimethyl-pyridine-2,4-diol (16.26 g, 69%). ESI-MS m/z calc. 216.97, found 218.0 (M+1)⁺.

### Step 2: 3-bromo-2,4-dichloro-5,6-dimethyl-pyridine

A mixture of 3-bromo-5,6-dimethyl-pyridine-2,4-diol (15.0 g, 68.8 mmol), POCl₃ (49 g, 30 mL, 322 mmol) and DMF (1.9 g, 2.0 mL, 26 mmol) was heated at 100° C. for 5 h. The mixture was quenched with ice-cold water (200 mL) followed by saturated aqueous sodium bicarbonate (200 mL) and extracted with ethyl acetate (2×500 mL). The combined organic layers were washed with brine (300 mL), dried over sodium sulfate, filtered and concentrated. Purification by silica gel chromatography (0-3% ethyl acetate/hexane) provided 3-bromo-2,4-dichloro-5,6-dimethyl-pyridine (12.24 g, 57%). ¹H NMR (400 MHz, DMSO-d₆) δ 2.37 (s, 3H), additional 3H obscured by solvent peak. ESI-MS m/z calc. 252.91, found 254.0 (M+1)⁺.

### Step 3: 2,4-dichloro-3-(2,5-dihydrofuran-3-yl)-5,6-dimethyl-pyridine

To a solution of 3-bromo-2,4-dichloro-5,6-dimethyl-pyridine (2.0 g, 6.4 mmol) in THF (35 mL) and water (5 mL) was added potassium phosphate (2.8 g, 13 mmol) followed by 2-(2,5-dihydro-3-furanyl)-4,4,5,5-tetramethyl-1,3,2-dioxaborolane (1.6 g, 8.2 mmol) and the mixture purged with argon. PdCl₂(dtbpf) (210 mg, 0.322 mmol) was added and the mixture heated at 60° C. for 3 h. The mixture was filtered and the filter washed with ethyl acetate. The organic layer was separated, dried over sodium sulfate, filtered and concentrated. Purification by silica gel chromatography (5-7% ethyl acetate/hexane) provided 2,4-dichloro-3-(2,5-dihydrofuran-3-yl)-5,6-dimethyl-pyridine (860 mg, 55%). ¹H NMR (400 MHz, DMSO-d₆) δ 6.12 (t, J=2.0 Hz, 1H), 4.81-4.72 (m, 2H), 4.68 (td, J=5.2, 2.2 Hz, 2H), 2.31 (s, 3H), additional 3H obscured by solvent peak. ESI-MS m/z calc. 243.02, found 244.08 (M+1)⁺.

### Step 4: 4-benzyloxy-2-chloro-3-(2,5-dihydrofuran-3-yl)-5,6-dimethyl-pyridine

To a solution of benzyl alcohol (0.4 mL, 3.8 mmol) in DMSO (8 mL) was added potassium tert-butoxide (500 mg, 4.46 mmol) portionwise at 0° C. and the mixture stirred for 30 min at 0° C. A solution of 2,4-dichloro-3-(2,5-dihydrofuran-3-yl)-5,6-dimethyl-pyridine (850 mg, 3.48 mmol) in DMSO (5 mL) was added and the mixture stirred at room temperature for 24 h. The mixture was quenched with ice-cold water (20 mL) and extracted with ethyl acetate (2×30 mL). The combined organic layers were washed with water (2×50 mL) and brine (50 mL), dried over sodium sulfate, filtered and concentrated. Purification by silica gel chromatography (5-7% ethyl acetate/hexane) provided 4-benzyloxy-2-chloro-3-(2,5-dihydrofuran-3-yl)-5,6-dimethyl-pyridine (776 mg, 70%). ¹H NMR (400 MHz, DMSO-d₆) δ 7.46-7.33 (m, 5H), 6.17-6.12 (m, 1H), 4.85 (s, 2H), 4.70 (s, 4H), 2.41 (s, 3H), 2.14 (s, 3H). ESI-MS m/z calc. 315.10, found 316.15 (M+1)⁺.

After Suzuki coupling of 4-benzyloxy-2-chloro-3-(2,5-dihydrofuran-3-yl)-5,6-dimethyl-pyridine with Intermediate-B, the alkene can be reduced using standard hydrogenation conditions to provide the corresponding tetrahydrofuran analog.

### Intermediate A-80

### 4-benzyloxy-2-chloro-3-(3,6-dihydro-2H-pyran-4-yl)-5,6-dimethyl-pyridine

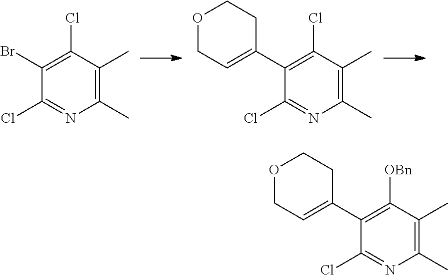

4-Benzyloxy-2-chloro-3-(3,6-dihydro-2H-pyran-4-yl)-5,6-dimethyl-pyridine was prepared in an analogous fashion as 4-benzyloxy-2-chloro-3-(2,5-dihydrofuran-3-yl)-5,6-dimethyl-pyridine using 3,6-dihydro-2H-pyran-4-boronic acid pinacol ester. ¹H NMR (400 MHz, DMSO-d₆) δ 7.44 (s, 1H), 7.43-7.34 (m, 4H), 5.79 (s, 1H), 4.89 (s, 2H), 4.20-4.13 (m, 2H), 3.75 (t, J=5.3 Hz, 2H), 2.40 (s, 3H), 2.24 (s, 2H), 2.14 (s, 3H). ESI-MS m/z calc. 329.12, found 330.23 (M+1)⁺.

After Suzuki coupling of 4-benzyloxy-2-chloro-3-(3,6-dihydro-2H-pyran-4-yl)-5,6-dimethyl-pyridine with Intermediate-B, the alkene can be reduced using standard hydrogenation conditions to provide the corresponding tetrahydropyran analog.

### Intermediate A-81

### 4-(4-benzyloxy-2-chloro-6-methyl-3-pyridyl)morpholine

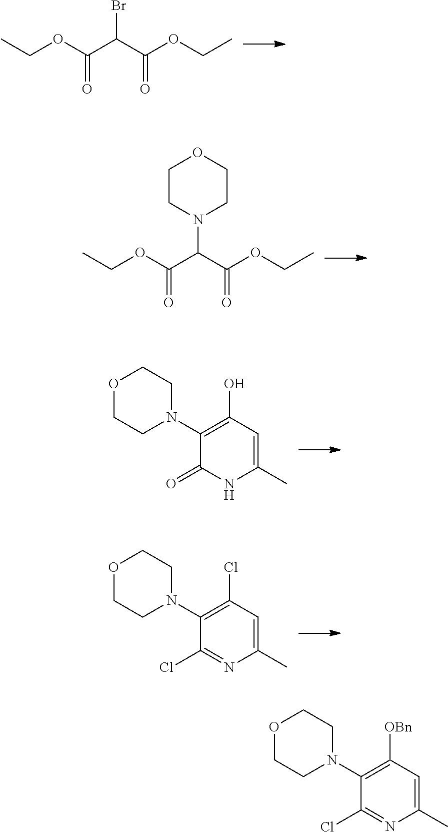

### Step 1: diethyl 2-morpholinopropanedioate

To a solution of diethyl 2-bromopropanedioate (25.0 g, 105 mmol) in acetonitrile (1 L) was added potassium carbonate (40.0 g, 289 mmol) followed by morpholine (25 g, 25 mL, 287 mmol) and the mixture stirred at room temperature for 3 h. The mixture was filtered, concentrated and purified by silica gel chromatography (10-15% ethyl acetate/hexane) to afford diethyl 2-morpholinopropanedioate (22.53 g, 88%). ¹H NMR (400 MHz, DMSO-d₆) δ 4.26 (s, 1H), 4.16 (q, J=7.1 Hz, 4H), 3.60-3.54 (m, 4H), 2.71-2.64 (m, 4H), 1.20 (t, J=7.1 Hz, 6H). ESI-MS m/z calc. 245.13, found 246.23 (M+1)⁺.

### Step 2: 4-hydroxy-6-methyl-3-morpholino-1H-pyridin-2-one

A mixture of ethyl (E)-3-aminobut-2-enoate (5.0 g, 39 mmol) and diethyl 2-morpholinopropanedioate (10.0 g, 40.8 mmol) was heated neat at 220° C. for 45 min. The residue was then dissolved in aqueous NaOH solution (200 mL of 2 M, 400 mmol) and heated at 130° C. for 24 h. The mixture was cooled and acidified with aqueous 1 N HCl. The precipitated solids were collected by filtration and dried in vacuo to afford crude 4-hydroxy-6-methyl-3-morpholino-1H-pyridin-2-one (3.12 g, 38%). ¹H NMR (400 MHz, DMSO-d₆) δ 10.96 (s, 1H), 5.67 (s, 1H), 3.66 (t, J=4.7 Hz, 4H), 2.96-2.86 (m, 4H), 2.06 (s. 3H). ESI-MS m/z cale. 210.10, found 211.39 (M+1)⁺

### Step 3: 4-(2,4-dichloro-6-methyl-3-pyridyl)morpholine

A solution of 4-hydroxy-6-methyl-3-morpholino-1H-pyridin-2-one (3.0 g, 14 mmol) and phenyl dichlorophosphate (28.2 g, 20.0 mL, 134 mmol) was heated at 180° C. for 1 h. The mixture was quenched with ice-cold water (50 mL) followed by saturated aqueous sodium bicarbonate (50 mL) and extracted with ethyl acetate (2×100 mL). The combined organic layers were washed with brine (100 mL), dried over sodium sulfate, filtered and concentrated. Purification by silica gel chromatography (5-8% ethyl acetate/hexane) provided 4-(2,4-dichloro-6-methyl-3-pyridyl)morpholine (1.56 g, 44%). ¹H NMR (400 MHz, DMSO-d₆) δ 7.46 (s, 1H), 3.70 (t, J=4.5 Hz, 4H), 3.09 (s, 4H), 2.39 (s, 3H). ESI-MS m/z calc. 246.03, found 247.31 (M+1)⁺.

### Step 4: 4-(4-benzyloxy-2-chloro-6-methyl-3-pyridyl)morpholine

To a solution of benzyl alcohol (800 mg, 7.40 mmol) in DMSO (15 mL) was added potassium tert-butoxide (902 mg, 8.04 mmol) portionwise at 0° C. and stirred for 30 min at 0° C. A solution of 4-(2,4-dichloro-6-methyl-3-pyridyl)morpholine (1.5 g, 6.1 mmol) in DMSO (10 mL) was added and the reaction mixture stirred at room temperature for 24 h. The mixture was quenched with ice-cold water (50 mL) and extracted with ethyl acetate (2×100 mL). The combined organic layers were washed with water (2×200 mL) and brine (100 mL), dried over sodium sulfate, filtered and concentrated. Purification by silica gel chromatography (20-50% ethyl acetate/hexane) provided 4-(4-benzyloxy-2-chloro-6-methyl-3-pyridyl)morpholine (1.03 g, 53%). ¹H NMR (400 MHz, DMSO-d₆) δ 7.49 (d, J=7.0 Hz, 2H), 7.44 (t, J=7.4 Hz, 2H), 7.40-7.33 (m, 1H), 7.11 (s, 1H), 5.22 (s, 2H), 3.62 (s, 4H), 2.96 (s, 4H), 2.35 (s, 3H). ESI-MS m/z calc. 318.11, found 319.28 (M+1)⁺.

### Intermediate A-82

### 2-(4-(benzyloxy-2-chloro-6-methylpyridin-3-yl)-1,3,4-oxadiazole

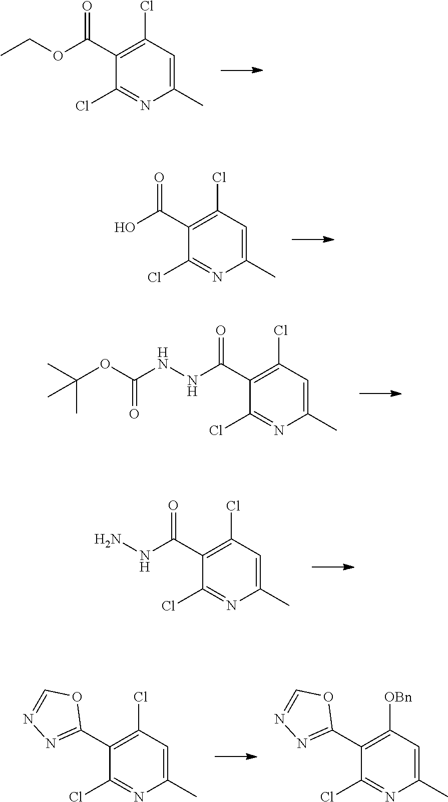 [SMILES: CCOC(=O)c1c(Cl)cc(C)nc1Cl ; Cc1cc(Cl)c(C(=O)O)c(Cl)n1 ; Cc1cc(Cl)c(C(=O)NNC(=O)OC(C)(C)C)c(Cl)n1 ; Cc1cc(Cl)c(C(=O)NN)c(Cl)n1 ; Cc1cc(Cl)c(-c2nnco2)c(Cl)n1 ; Cc1cc(OCc2ccccc2)c(-c2nnco2)c(Cl)n1]

### Step 1: 2,4-dichloro-6-methyl-pyridine-3-carboxylic acid

To a solution of ethyl 2,4-dichloro-6-methyl-pyridine-3-carboxylate (1.0 g, 4.3 mmol) in ethanol (10 mL) was added LiOH (6.5 mL of 2 M, 13 mmol). The mixture stirred at room temperature for 2 h, then heated at 75° C. for 20 h. Additional LiOH (5 mL of 2 M, 10 mmol) was added and the mixture heated at 80° C. for 3 h. The mixture was cooled and concentrated, then acidified with 2 M HCl. The mixture was extracted with ethyl acetate (×3), then dried and filtered using Whatman 1PS hydrophobic phase separator filter paper. The filtrate was concentrated to provide 2,4-dichloro-6-methyl-pyridine-3-carboxylic acid (823 mg, 94%). ¹H NMR (400 MHz, CD₃OD) δ 7.44 (d, J=0.7 Hz, 1H), 2.53 (d, J=0.6 Hz, 3H).

### Step 2: tert-butyl N-[(2,4-dichloro-6-methyl-pyridine-3-carbonyl)amino]carbamate

A mixture of 2,4-dichloro-6-methyl-pyridine-3-carboxylic acid (7.0 g, 34 mmol) and SOCl₂ (24.5 g, 15 mL, 206 mmol) was refluxed for 2 h. The mixture was concentrated and the residue was dissolved in benzene and evaporated under reduced pressure to provide the corresponding acid chloride (9 g). A solution of the acid chloride (9 g) in dichloromethane (40 mL) was added slowly to a mixture of tert-butyl carbazate (4.5 g, 34 mmol) and TEA (10.9 g, 15.0 mL, 108 mmol) in dichloromethane (80 mL) at 0° C. The mixture was stirred for 2 h, then diluted with water (60 mL) and extracted with ethyl acetate (2×300 mL). The combined organic layers were washed with aqueous sodium bicarbonate solution (100 mL) and brine (100 mL), dried over magnesium sulfate, filtered and concentrated. Purification by silica gel chromatography (25-30% ethyl acetate/hexane) provided tert-butyl N-[(2,4-dichloro-6-methyl-pyridine-3-carbonyl)amino]carbamate (6.3 g, 54%). ¹H NMR (400 MHz, DMSO-d₆) δ 10.32 (s, 1H), 9.17 (s, 1H), 7.59 (s, 1H), 2.48 (s, 3H), 1.43 (s, 9H). ESI-MS m/z calc. 319.05, found 320.2 (M+1)⁺.

### Step 3: 2,4-dichloro-6-methyl-pyridine-3-carbohydrazide

Hydrochloric acid (in dioxane) (34 mL of 4 M, 136.00 mmol) was added to tert-butyl N-[(4,6-dichloro-2-methyl-pyridine-3-carbonyl)amino]carbamate (2.0 g, 5.8 mmol) and the mixture stirred at room temperature for 2 h. The mixture was concentrated to provide 4,6-dichloro-2-methyl-pyridine-3-carbohydrazide (1.27 g, 94%) as a white solid. ¹H NMR (400 MHz, CD₃OD) δ 7.53 (s, 1H), 2.55 (s, 3H). ESI-MS m/z calc. 219.00, found 219.9 (M+1)⁺.

### Step 4: 2-(2,4-dichloro-6-methyl-3-pyridyl)-1,3,4-oxadiazole

A solution of 4,6-dichloro-2-methyl-pyridine-3-carbohydrazide (1.27 g, 5.44 mmol) in triethyl orthoformate (5.3 g, 6.0 mL, 36 mmol) was heated to 140° C. for 17 h. After cooling to room temperature, the mixture was poured into water (10 mL) and extracted with ethyl acetate (×3). The combined organic layers were washed with brine, dried over sodium sulfate, filtered and concentrated. Purification by silica gel chromatography (5-20% ethyl acetate/heptane) provided 2-(4,6-dichloro-2-methyl-3-pyridyl)-1,3,4-oxadiazole (0.75 g, 59%). ¹H NMR (400 MHz, CDCl₃) δ 8.64 (s, 1H), 7.33 (s, 1H), 2.63 (s, 3H). ESI-MS m/z calc. 228.98, found 229.85 (M+1)⁺.

### Step 5: 2-(4-(benzyloxy)-2-chloro-6-methylpyridin-3-yl)-1,3,4-oxadiazole

A solution of benzyl alcohol (350 mg, 0.34 mL, 3.3 mmol) in 2-MeTHF (15 mL) under argon was cooled to 0° C. and NaH in mineral oil (155 mg, 60% w/w, 3.9 mmol) was added in one portion. The mixture was stirred at 0° C. for 30 min before being cooled to −25° C. A solution of 2-(4,6-dichloro-2-methyl-3-pyridyl)-1,3,4-oxadiazole (0.75 g, 3.2 mmol) in 2-MeTHF (3 mL) was added and the reaction was stirred for 3 h at room temperature. The mixture was partitioned between water and ethyl acetate and the layers separated. The aqueous layer was extracted with ethyl acetate (×3). The combined organic layers were washed with brine, dried over sodium sulfate, filtered and concentrated. Purification by silica gel chromatography (0-40% ethyl acetate/heptane) provided first 2-(2-benzyloxy-4-chloro-6-methyl-3-pyridyl)-1,3,4-oxadiazole (276 mg, 25%) as a clear oil. ¹H NMR (400 MHz, CDCl₃) δ 8.43 (s, 1H), 7.24-7.16 (m, 5H), 6.84 (s, 1H), 5.32 (s, 2H), 2.39 (s, 3H). ESI-MS m/z calc. 301.06, found 302.05 (M+1)⁺, followed by the desired 2-(4-benzyloxy-2-chloro-6-methyl-3-pyridyl)-1,3,4-oxadiazole (644 mg, 66%) as a white crystalline solid. ¹H NMR (400 MHz, CDCl₃) δ 8.58 (s, 1H), 7.39-7.26 (m, 5H), 6.79 (s, 1H), 5.17 (s, 2H), 2.56 (s, 3H). ESI-MS m/z calc. 301.06, found 302.04 (M+1)⁺

### Intermediate A-83

### 4-benzyloxy-2-chloro-6-methyl-3-(1-methylpyrazol-3-yl)pyridine

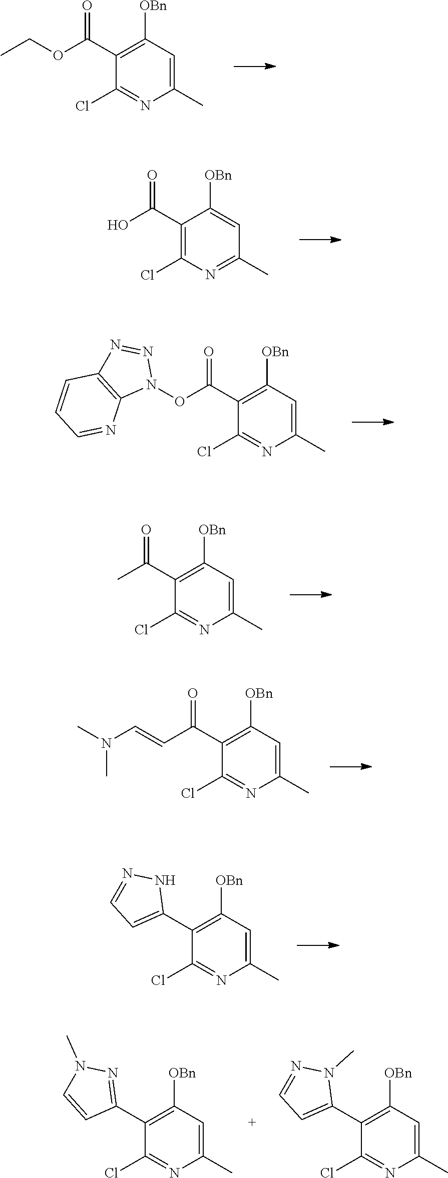

### Step 1: 4-benzyloxy-2-chloro-6-methyl-pyridine-3-carboxylic acid

To a stirring solution of ethyl 4-benzyloxy-2-chloro-6-methyl-pyridine-3-carboxylate (300 mg, 0.981 mmol) in THF (3 mL) and MeOH (1 mL) was added aqueous NaOH (4.9 mL of 1.0 M, 4.9 mmol) and the mixture stirred at 65° C. overnight. The mixture was diluted with ethyl acetate and washed with 1 N HCl, water and brine. Then organic layer was dried over sodium sulfate, filtered and concentrated. Purification by reverse phase HPLC (C18, 1-99% acetonitrile in water/5 mM HCl) provided 4-benzyloxy-2-chloro-6-methyl-pyridine-3-carboxylic acid (203 mg, 75%). ESI-MS m/z cale. 277.05, found 278.1 (M+1)⁺.

### Step 2: triazolo[4,5-b]pyridin-3-yl 4-benzyloxy-2-chloro-6-methyl-pyridine-3-carboxylate

To a stirring solution of 4-benzyloxy-2-chloro-6-methyl-pyridine-3-carboxylic acid (500 mg, 1.35 mmol) in acetonitrile (4 mL) was added HATU (720 mg, 1.89 mmol) followed by DIPEA (455 mg, 3.52 mmol). The mixture was stirred for 16 h at room temperature under argon atmosphere then partitioned between ethyl acetate and water. The aqueous layer was separated and extracted with additional ethyl acetate (×2). The combined organic layers were dried over magnesium sulfate, filtered and concentrated. The residue was then stirred with ethyl acetate (25 mL) for 30 min and the resulting solid collected by filtration to provide triazolo[4,5-b]pyridin-3-yl 4-benzyloxy-2-chloro-6-methyl-pyridine-3-carboxylate (623 mg, 88%). ESI-MS m/z calc. 395.08, found 394.1 (M−1)⁻. ¹H NMR (400 MHz, DMSO-d₆) δ 8.88 (d, J=3.9 Hz, 1H), 8.77 (d, J=7.8 Hz, 1H), 7.70 (dd, J=8.4 Hz, 4.3 Hz, 1H), 7.60-7.54 (m, 3H), 7.46-7.36 (m, 3H), 5.50 (s, 2H), 2.57 (s, 3H).

### Step 3: 1-(4-benzyloxy-2-chloro-6-methyl-3-pyridyl)ethanone

To a stirring solution of triazolo[4,5-b]pyridin-3-yl 4-benzyloxy-2-chloro-6-methyl-pyridine-3-carboxylate (330 mg, 0.628 mmol) in THF (6 mL) was added MeLi (in solution in THF) (1.2 mL of 1.6 M, 1.9 mmol) at −40° C. dropwise over a period of 5 min. The mixture was stirred for 30 min at the same temperature then quenched with saturated aqueous ammonium chloride solution (10 mL). Water (5 mL) was added and the aqueous phase was extracted with ethyl acetate (2×20 mL). The combined organic layers were dried over magnesium sulfate, filtered and concentrated. Purification by silica gel chromatography (0-50% ethyl acetate/heptane) provided 1-(4-benzyloxy-2-chloro-6-methyl-3-pyridyl)ethanone (136 mg, 47%). ESI-MS m/z calc. 275.07, found 276.03 (M+1)⁺. ¹H NMR (400 MHz, CDCl₃) δ 7.34-7.24 (m, 5H), 6.62 (s, 1H), 5.05 (s, 2H), 2.43 (s, 3H), 2.40 (s, 3H).

### Step 4: (E)-1-(4-benzyloxy-2-chloro-6-methyl-3-pyridyl)-3-(dimethylamino)prop-2-en-1-one

To a stirring solution of 1-(4-benzyloxy-2-chloro-6-methyl-3-pyridyl)ethanone (1.3 g, 4.5 mmol) in DMF (30 mL) was added DMF-DMA (1.11 g, 1.24 mL, 9.33 mmol) and the mixture heated at 80° C. for 3 h. The mixture was diluted with water (300 mL) and extracted with ethyl acetate (3×100 mL). The combined organic layers were dried over magnesium sulfate, filtered and concentrated to provide (E)-1-(4-benzyloxy-2-chloro-6-methyl-3-pyridyl)-3-(dimethylamino)prop-2-en-1-one (1.7 g, 92%). ESI-MS m/z calc. 330.11, found 331.11 (M+1)⁺. ¹H NMR (400 MHz, DMSO-d₆) δ 7.43 (d, J=4.8 Hz, 1H), 7.39-7.33 (m, 4H), 7.11 (s, 1H), 5.28-5.21 (m, 3H), 5.12 (d, J=12.9 Hz, 1H), 3.03 (s, 3H), 2.80 (s, 3H), 2.41 (s, 3H).

### Step 5: 4-benzyloxy-2-chloro-6-methyl-3-(1H-pyrazol-5-yl)pyridine

To a stirring solution of (E)-1-(4-benzyloxy-2-chloro-6-methyl-3-pyridyl)-3-(dimethylamino)prop-2-en-1-one (90 mg, 0.23 mmol) in ethanol (3 mL) was added hydrazine in THF (0.6 mL of 1 M, 0.6 mmol) at room temperature. The mixture was refluxed at 90° for 16 h. The mixture was concentrated and purified by silica gel chromatography (80% ethyl acetate/hexane) to provide 4-benzyloxy-2-chloro-6-methyl-3-(1H-pyrazol-5-yl)pyridine (40 mg, 55%). ESI-MS m/z calc. 299.08, found 300.1 (M+1)⁺.

### Step 6: 4-benzyloxy-2-chloro-6-methyl-3-(1-methylpyrazol-3-yl)pyridine and 4-benzyloxy-2-chloro-6-methyl-3-(2-methylpyrazol-3-yl)pyridine

To a stirred solution of 4-benzyloxy-2-chloro-6-methyl-3-(1H-pyrazol-5-yl)pyridine (30 mg, 0.10 mmol) in DMF (5 mL) was added potassium carbonate (45 mg, 0.33 mmol) followed by MeI (46 mg, 0.02 mL, 0.32 mmol) at room temperature. The mixture was stirred for 16 h at room temperature. The mixture was diluted with water (500 mL) and extracted with ethyl acetate (200 mL). The organic layer was dried over magnesium sulfate, filtered and concentrated. Purification by silica gel chromatography (20-30% ethyl acetate/hexane) provided 4-benzyloxy-2-chloro-6-methyl-3-(2-methylpyrazol-3-yl)pyridine (1.01 g, 30%). ¹H NMR (400 MHz, DMSO-d₆) δ 7.48 (d, J=1.9 Hz, 1H), 7.36-7.25 (m, 6H), 6.29 (d, J=1.9 Hz, 1H), 5.30-5.17 (m, 2H), 3.55 (s, 3H), 2.48 (s, 3H). ESI-MS m/z calc. 313.10, found 314.17 (M+1)⁺, and 4-benzyloxy-2-chloro-6-methyl-3-(1-methylpyrazol-3-yl)pyridine (440 mg, 13%). ¹H NMR (400 MHz, DMSO-d₆) δ 7.73 (d, J=2.2 Hz, 1H), 7.38-7.30 (m, 5H), 7.14 (s, 1H), 6.28 (d, J=2.2 Hz, 1H), 5.20 (s, 2H), 3.87 (s, 3H), 2.43 (s, 3H). ESI-MS m/z calc. 313.10, found 314.17 (M+1)⁺.

### Intermediate A-84

### 4-(4-benzyloxy-2-chloro-5,6-dimethyl-3-pyridyl)morpholine

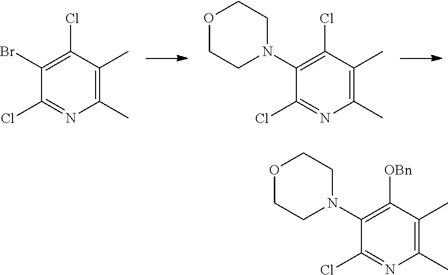

### Step 1: 4-(2,4-dichloro-5,6-dimethyl-3-pyridyl)morpholine

To a solution of 3-bromo-2,4-dichloro-5,6-dimethyl-pyridine (4.0 g, 13 mmol) in 2-MeTHF (50 mL) was added sodium tert-butoxide (1.8 g, 19 mmol) followed by morpholine (1.3 g, 1.3 mL, 15 mmol) and the mixture purged with argon. Xantphos (750 mg, 1.30 mmol) and Pd₂(dba)₃ (590 mg, 0.644 mmol) were added and the mixture heated at 100° C. for 48 h. The mixture was filtered, washed with ethyl acetate and the filtrate concentrated. Purification by silica gel chromatography (5-7% ethyl acetate/hexane) provided 4-(2,4-dichloro-5,6-dimethyl-3-pyridyl)morpholine (1.62 g, 30%). ESI-MS m/z calc. 260.05, found 261.2 (M+1)⁺.

### Step 2: 4-(4-benzyloxy-2-chloro-5,6-dimethyl-3-pyridyl)morpholine

To a solution of benzyl alcohol (520 mg, 0.50 mL, 4.8 mmol) in DMF (10 mL) was added sodium hydride (dispersion in mineral oil) (200 mg, 60% w/w, 5.0 mmol) portionwise at 0° C. and the mixture stirred for 30 min at 0° C. A solution of 4-(2,4-dichloro-5,6-dimethyl-3-pyridyl)morpholine (1.6 g, 3.8 mmol) in DMF (5 mL) was added to the reaction mixture and heated at 100° C. for 24 h. The mixture was quenched with ice-cold water (30 mL) and extracted with ethyl acetate (2×30 mL). The combined organic layers were washed with water (2×50 mL) and brine (30 mL), dried over sodium sulfate, filtered and concentrated. Purification by silica gel chromatography (5-7% ethyl acetate/hexane) provided 4-(4-benzyloxy-2-chloro-5,6-dimethyl-3-pyridyl)morpholine (915 mg, 72%). ¹H NMR (400 MHz, DMSO-d₆) δ 7.53-7.35 (m, 5H), 5.00 (s, 2H), 3.66 (t, J=4.5 Hz, 4H), 3.10 (t, J=4.6 Hz, 4H), 2.34 (s, 3H), 2.07 (s, 3H). ESI-MS m/z calc. 332.13, found 333.24 (M+1)⁺.

### Intermediate B-1

### [2-(3,4-difluoro-2-methyl-phenoxy)-3-quinolyl]boronic acid

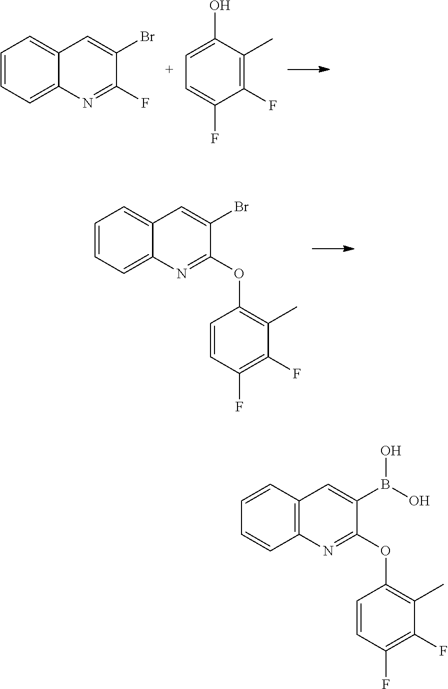

### Step 1: 3-bromo-2-(3,4-difluoro-2-methyl-phenoxy)quinoline

To a mixture of 3-bromo-2-fluoro-quinoline (500 mg, 2.21 mmol) and 3,4-difluoro-2-methyl-phenol (478 mg, 3.32 mmol) in DMSO (12 mL) was added cesium carbonate (1.8 g, 5.5 mmol). The resulting mixture was stirred at 55° C. for 4 h. The reaction was cooled to room temperature, diluted with ethyl acetate, washed with water and brine. The organic layer was dried over sodium sulfate, filtered and concentrated under reduced pressure. Purification using silica gel chromatography (0-20% ethyl acetate/hexanes) provided 3-bromo-2-(3,4-difluoro-2-methyl-phenoxy)quinoline (596 mg, 77%). ¹H NMR (400 MHz, DMSO-d₆) δ 8.89 (s, 1H), 7.95 (dd, J=8.1, 1.4 Hz, 1H), 7.69 (ddd, J=8.4, 6.9, 1.5 Hz, 1H), 7.60 (dd, J=8.1, 1.0 Hz, 1H), 7.54 (ddd, J=8.1, 6.8, 1.3 Hz, 1H), 7.40 (m, 1H), 7.17 (ddd, J=9.1, 4.4, 2.1 Hz, 1H), 2.07 (d, J=2.2 Hz, 3H). ESI-MS m/z calc. 348.99, found 350.0 (M+1)⁺.

### Step 2: [2-(3,4-difluoro-2-methyl-phenoxy)-3-quinolyl]boronic acid

A solution of 3-bromo-2-(3,4-difluoro-2-methyl-phenoxy)quinoline (600 mg, 1.71 mmol) in THF (4 mL) at −78° C. was treated dropwise with n-BuLi (750 μL of 2.5 M in hexanes, 1.9 mmol). The reaction mixture was stirred for 30 min then treated dropwise with triisopropyl borate (550 μL, 2.40 mmol). The mixture was stirred for 30 min at −78° C., then removed from the cooling bath and quenched with saturated aqueous ammonium chloride. The mixture was diluted with diethyl ether and the layers separated. The aqueous layer was extracted with additional diethyl ether (3×), and the combined organic layers dried over magnesium sulfate, filtered and concentrated to provide [2-(3,4-difluoro-2-methyl-phenoxy)-3-quinolyl]boronic acid (505 mg, 94%). ESI-MS m/z calc. 315.09, found 316.2 (M+1)⁺. ¹H NMR (400 MHz, DMSO-d₆) δ 8.51 (d, J=4.8 Hz, 1H), 8.36 (s, 2H), 7.94 (dd, J=8.1, 1.5 Hz, 1H), 7.66-7.57 (m, 1H), 7.54 (d, J=8.7 Hz, 1H), 7.46 (ddd, J=8.1, 6.8, 1.4 Hz, 1H), 7.41-7.28 (m, 1H), 7.13 (m, 1H), 2.06 (dd, J=5.4, 2.3 Hz, 3H).

### Intermediate B-2

### 4-(3,4-Difluoro-2-methyl-phenoxy)-5-(4,4,5,5-tetramethyl-1,3,2-dioxaborolan-2-yl)-2-(trifluoromethyl)pyridine

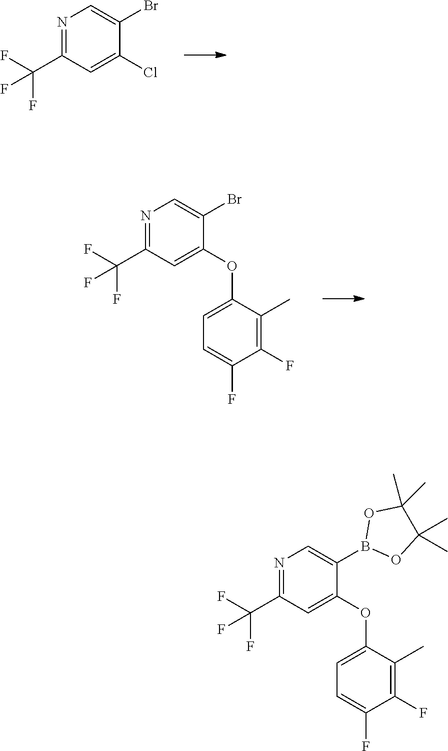 [SMILES: FC(F)(F)c1cc(Cl)c(Br)cn1 ; Cc1c(Oc2cc(C(F)(F)F)ncc2Br)ccc(F)c1F ; Cc1c(Oc2cc(C(F)(F)F)ncc2B2OC(C)(C)C(C)(C)O2)ccc(F)c1F]

### Step 1: 5-bromo-4-(3,4-difluoro-2-methyl-phenoxy)-2-(trifluoromethyl)pyridine

3,4-Difluoro-2-methyl-phenol (588 mg, 4.08 mmol), 5-bromo-4-chloro-2-(trifluoromethyl)pyridine (1.00 g, 3.84 mmol), cesium carbonate (2.68 g, 8.23 mmol) were combined in DMF (5.0 mL) and stirred at 100° C. (microwave irradiation) for 2 h. Purification by silica gel chromatotgraphy provided 5-bromo-4-(3,4-difluoro-2-methyl-phenoxy)-2-(trifluoromethyl)pyridine (1.260 g, 82%) as a pale orange solid ESI-MS m/z calc. 366.96, found 367.93 (M+1)⁺

### Step 2: 4-(3,4-Difluoro-2-methyl-phenoxy)-5-(4,4,5,5-tetramethyl-1,3,2-dioxaborolan-2-yl)-2-(trifluoromethyl)pyridine

4-(3,4-Difluoro-2-methyl-phenoxy)-5-(4,4,5,5-tetramethyl-1,3,2-dioxaborolan-2-yl)-2-(trifluoromethyl)pyridine was prepared from 5-bromo-4-chloro-2-(trifluoromethyl)pyridine and 3,4-difluoro-2-methyl-phenol using a procedure analogous to that found in Intermediate B-1, step 2. ESI-MS m/z calc. 415.14, found 334.157 (M−82)⁺.

### Intermediate B-3

### 2-[4-tert-butyl-2-(4-fluoro-2-methoxy-phenoxy)-6-methyl-phenyl]-4,4,5,5-tetramethyl-1,3,2-dioxaborolane

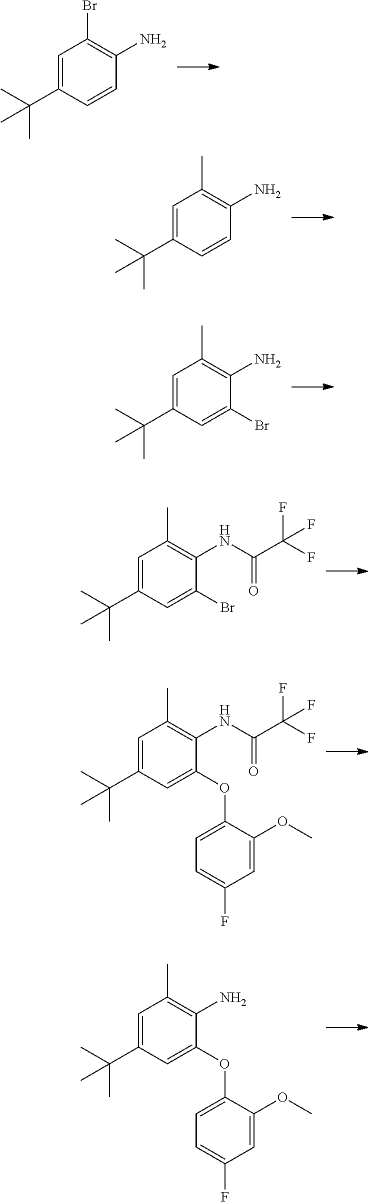

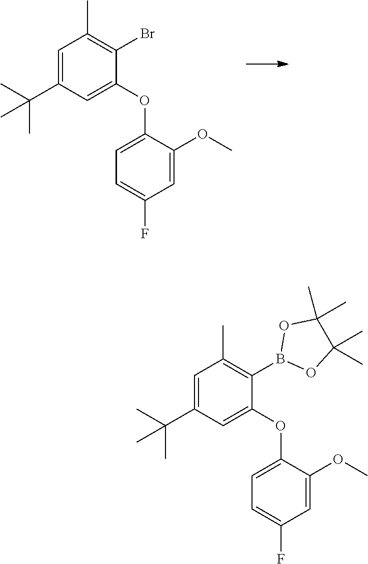

### Step 1: 4-tert-butyl-2-methyl-aniline

To a solution of 2-bromo-4-tert-butyl-aniline (25.0 g, 110 mmol) in dioxane (750 mL) and water (85 mL) was added methylboronic acid (32.8 g, 548 mmol), tricyclohexylphosphine (6.3 g, 22.5 mmol) and potassium phosphate (70 g, 330 mmol). The mixture was bubbled with nitrogen for 5 min then palladium acetate (2.5 g, 11 mmol) was added. The mixture was heated at 110° C. for 18 h. The crude was filtered on Celite® and washed with dichloromethane (300 mL). The filtrate was washed with brine (2×200 mL), dried with anhydrous sodium sulfate, filtered and concentrated under reduced pressure. Purification by silica gel chromatography (330 g silica, 0-30% ethyl acetate/heptane) provided 4-tert-butyl-2-methyl-aniline (16.9 g, 94%) as a dark oil. ESI-MS m/z calc. 163.14, found 164.2 (M+1)⁺. ¹H NMR (400 MHz, CDCl₃) δ 7.12-7.05 (m, 2H), 6.66 (d, J=7.8 Hz, 1H), 3.52 (br s, 2H), 2.21 (s, 3H), 1.31 (s, 9H).

### Step 2: 2-bromo-4-tert-butyl-6-methyl-aniline

N-Bromosuccinimide (20.5 g, 115 mmol) was slowly added to a cold (−30° C.) solution of 4-tert-butyl-2-methyl-aniline (19.57 g, 119.9 mmol) in dichloromethane (1.2 L). The reaction mixture was stirred at −30° C. for 3 h then reaction was quenched with water (700 mL). Once warmed to room temperature, the organic layer was separated, dried over sodium sulfate and concentrated under reduced pressure to afford crude 2-bromo-4-tert-butyl-6-methyl-aniline (29.14 g, 96%) as a dark oil. The crude product was used for the next step without any further purification. ESI-MS m/z calc. 241.05, found 242.1 (M+1)⁺. ¹H NMR (400 MHz, CDCl₃) δ 7.32 (d, J=2.2 Hz, 1H), 7.06-7.02 (m, 1H), 4.05-3.89 (m, 2H), 2.24 (s, 3H), 1.29 (s, 9H).

### Step 3: N-(2-bromo-4-tert-butyl-6-methyl-phenyl)-2,2,2-trifluoro-acetamide

Trifluoroacetic anhydride (20 mL, 144 mmol) was added dropwise to a solution of 2-bromo-4-tert-butyl-6-methyl-aniline (29.14 g, 114.6 mmol) and triethylamine (24 mL, 172 mmol) in dichloromethane (300 mL) at 0° C. The reaction mixture was stirred at room temperature for 3 h, then water (200 mL) was added and mixture was extracted using dichloromethane (3×100 mL). Organic layer was washed with aqueous saturated sodium bicarbonate (2×150 mL), dried over sodium sulfate, filtered and concentrated under reduced pressure to afford N-(2-bromo-4-tert-butyl-6-methyl-phenyl)-2,2,2-trifluoro-acetamide (40.05 g, 100%) as brown solid. The crude material was used for the next step without any further purification. ESI-MS m/z calc. 337.03, found 338.0 (M+1)⁺. ¹H NMR (400 MHz, CDCl₃) δ 7.64-7.56 (m, 1H), 7.51 (d, J=1.5 Hz, 1H), 7.26 (s, 1H), 2.32 (s, 3H), 1.33 (s, 9H).

### Step 4: N-[4-tert-butyl-2-(4-fluoro-2-methoxy-phenoxy)-6-methyl-phenyl]-2,2,2-trifluoro-acetamide

A mixture of N-(2-bromo-4-tert-butyl-6-methyl-phenyl)-2,2,2-trifluoro-acetamide (87.8 g, 235 mmol), 4-fluoro-2-methoxy-phenol (39.9 g, 32 mL, 281 mmol), cesium carbonate (230 g, 706 mmol) and NN-dimethylglycine (24.2 g, 235 mmol) in 1,4-dioxane (920 mL) was degassed with argon for 10 min, then copper (I) iodide (13.4 g, 70.4 mmol) added. The reaction was warmed to 60° C. under argon for 20 h, then allowed to cool. The reaction was filtered through Celite® and concentrated. The Celite® was washed with warm water (1.2 l) and ethyl acetate (600 mL), which were combined with the concentrate, and the phases separated. The aqueous was extracted with ethyl acetate (3×400 mL). The combined organics were washed with water (3×400 mL) then brine (400 mL), dried over sodium sulfate and silica gel, filtered and concentrated. The crude was boiled in heptane (200 mL), allowed to cool slowly to room temperature, then cooled to 0° C. The solid was collected by filtration to provide N-[4-tert-butyl-2-(4-fluoro-2-methoxy-phenoxy)-6-methyl-phenyl]-2,2,2-trifluoro-acetamide (70.3 g, 75%). ESI-MS m/z calc. 399.15, found 400.16 (M+1)⁺. ¹H NMR (301 MHz, CDCl₃) δ 7.91 (br s, 1H), 7.00 (d, J=1.4 Hz, 1H), 6.94 (dd, J=8.9, 5.4 Hz, 1H), 6.73-6.69 (m, 2H), 6.61 (m, 1H), 3.80 (s, 3H), 2.27 (s, 3H), 1.21 (s, 9H).

### Step 5: 4-tert-butyl-2-(4-fluoro-2-methoxy-phenoxy)-6-methyl-aniline

To a solution of N-[4-tert-butyl-2-(4-fluoro-2-methoxy-phenoxy)-6-methyl-phenyl]-2,2,2-trifluoro-acetamide (70 g, 175 mmol) in ethanol (875 mL) was added NaOH (350 mL of 2 M, 700 mmol), and the mixture was stirred under reflux for 3 h. The reaction was allowed to cool, then the ethanol removed under reduced pressure. The residue was diluted with water (300 mL) then extracted with CPME (3×250 mL). The combined organics were washed with 1 N NaOH (200 mL) then brine (200 mL), dried over sodium sulfate, filtered and concentrated to provide 4-tert-butyl-2-(4-fluoro-2-methoxy-phenoxy)-6-methyl-aniline (54.9 g, 100%). ESI-MS m/z calc. 303.16, found 304.14 (M+1)⁺. ¹H NMR (301 MHz, CDCl₃) δ 6.87 (d, J=1.7 Hz, 1H), 6.79-6.71 (m, 2H), 6.67 (d, J=2.1 Hz, 1H), 6.59-6.52 (m, 1H), 3.87 (s, 3H), 2.22 (s, 3H), 1.20 (s, 9H).

### Step 6: 2-bromo-5-tert-butyl-1-(4-fluoro-2-methoxy-phenoxy)-3-methyl-benzene

To a slurry of copper (II) bromide (48.6 g, 218 mmol), lithium bromide (45.4 g, 523 mmol), and tert-butyl nitrite (19.7 g, 16 mL, 172 mmol) in acetonitrile (700 mL) was added a solution of 4-tert-butyl-2-(4-fluoro-2-methoxy-phenoxy)-6-methyl-aniline (54.9 g, 174 mmol) in acetonitrile (420 mL) at room temperature over 40 min. The reaction was warmed to 60° C. for 4 h then allowed to cool. The reaction was quenched with 1 N HCl (800 mL) and extracted with CPME (3×300 mL). The combined organics were washed with water (2×300 mL) and brine (300 mL), dried over sodium sulfate and concentrated. Purification by silica gel chromatography (0-2% ethyl acetate/heptane) provided 2-bromo-5-tert-butyl-1-(4-fluoro-2-methoxy-phenoxy)-3-methyl-benzene (41.3 g, 62%). ¹H NMR (400 MHz, CDCl₃) δ 6.99 (d, J=2.3 Hz, 1H), 6.79-6.72 (m, 2H), 6.63 (d, J=2.3 Hz, 1H), 6.61-6.56 (m, 1H), 3.84 (s, 3H), 2.45 (s, 3H), 1.20 (s, 9H).

### Step 7: 2-[4-tert-butyl-2-(4-fluoro-2-methoxy-phenoxy)-6-methyl-phenyl]-4,4,5,5-tetramethyl-1,3,2-dioxaborolane

n-BuLi (1.2 mL of 2.5 M, 3.0 mmol) was slowly added to a solution of 2-bromo-5-tert-butyl-1-(4-fluoro-2-methoxy-phenoxy)-3-methyl-benzene (1.0 g, 2.7 mmol) in THF (20 mL) at −78° C. The reaction mixture was stirred for 15 min then a pre-cooled solution of 2-isopropoxy-4,4,5,5-tetramethyl-1,3,2-dioxaborolane (684 mg, 0.75 mL, 3.68 mmol) in THF (5 mL) was slowly added. The reaction mixture was stirred for 1.5 h at −78° C., then warmed to 0° C. Reaction mixture was quenched with water (20 mL), poured into 1:1 saturated sodium chloride/water solution (50 mL) and extracted using ethyl acetate (3×50 mL). The organic layers were combined, dried over sodium sulfate, filtered and concentrated under reduced pressure. The residue was purified by silica gel chromatography (0-20% ethyl acetate/heptane) to afford 2-[4-tert-butyl-2-(4-fluoro-2-methoxy-phenoxy)-6-methyl-phenyl]-4,4,5,5-tetramethyl-1,3,2-dioxaborolane (890 mg, 79%) as clear oil. ¹H NMR (400 MHz, CDCl₃) δ 6.96 (d, J=1.0 Hz, 1H), 6.76-6.62 (m, 3H), 6.59-6.47 (m, 1H), 3.89 (s, 3H), 2.45 (s, 3H), 1.24 (s, 9H), 1.24 (s, 12H).

### Intermediate B-4

### [2-(4-fluoro-2-methyl-phenoxy)-4-methyl-5-(trifluoromethyl)-3-pyridyl]boronic acid

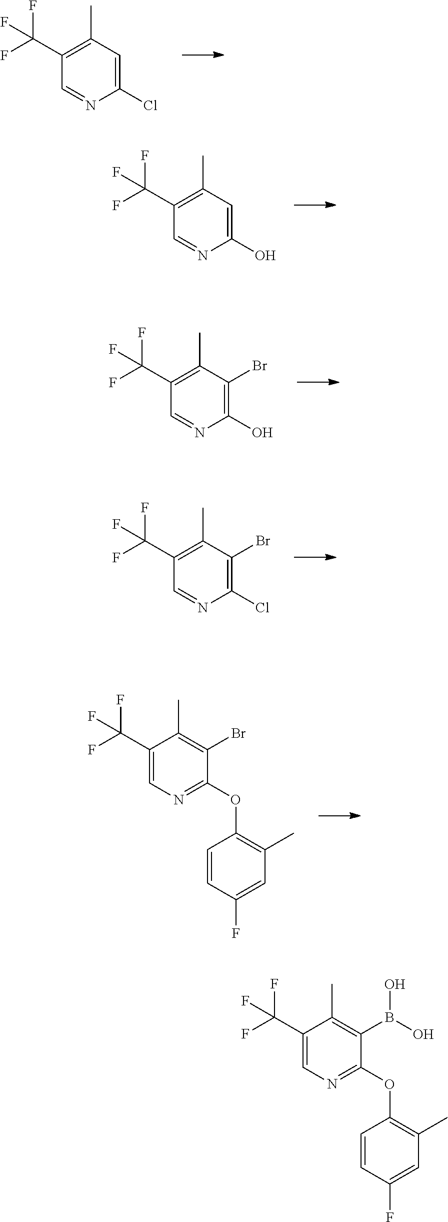 [SMILES: Cc1cc(Cl)ncc1C(F)(F)F ; Cc1cc(O)ncc1C(F)(F)F ; Cc1c(C(F)(F)F)cnc(O)c1Br ; Cc1c(C(F)(F)F)cnc(Cl)c1Br ; Cc1cc(F)ccc1Oc1ncc(C(F)(F)F)c(C)c1Br ; Cc1cc(F)ccc1Oc1ncc(C(F)(F)F)c(C)c1B(O)O]

### Step 1: 4-methyl-5-(trifluoromethyl)pyridin-2-ol

A mixture of 2-chloro-4-methyl-5-(trifluoromethyl)pyridine (5.0 g, 25.6 mmol), aqueous hydrochloric acid (20 mL of 37% w/v, 203 mmol), 1,4-dioxane (40 mL) and water (20 mL) was stirred at 95° C. for 48 h, then stirred at 80° C. for an additional 24 h. The mixture was diluted with 2 M aqueous sodium hydroxide (100 mL) and saturated aqueous sodium bicarbonate (100 mL), then extracted with ethyl acetate (4×200 mL). The combined organic layers were washed with water (100 mL) and brine (100 mL), dried over sodium sulfate, filtered and evaporated under reduced pressure provided 4-methyl-5-(trifluoromethyl)pyridin-2-ol (4.82 g, 98%) as a white solid. ESI-MS m/z calc. 177.04, found 178.2 (M+1)⁺. ¹H NMR (400 MHz, CDCl₃) δ 13.21 (br s, 1H), 7.70 (s. 1H), 6.44 (s, 1H), 2.35 (s, 3H). ¹⁹F NMR (377 MHz, CDCl₃) δ −61.29 (s, 3F).

### Step 2: 3-bromo-4-methyl-5-(trifluoromethyl)pyridin-2-ol

To a solution of 4-methyl-5-(trifluoromethyl)pyridin-2-ol (4.8 g, 25 mmol) in acetic acid (65 mL) was added bromine (8.7 g, 2.8 mL, 54 mmol). The reaction mixture was stirred at room temperature for 65 h, then poured onto a stirring mixture of sodium carbonate (90 g), water (400 mL) and sodium thiosulfate pentahydrate (17 g). The aqueous layer was extracted with ethyl acetate (3×250 mL). The organic layers were combined and washed with water (100 mL) and brine (100 mL), dried over sodium sulfate, filtered and evaporated under reduced pressure provided a beige solid (6.05 g). The crude product was triturated in 1:1 MTBE/heptane (30 mL) and filtered. The solid was washed with 1:1 MTBE/heptane (15 mL) and dried provided 3-bromo-4-methyl-5-(trifluoromethyl)pyridin-2-ol (5.38 g, 84%) as a white solid. ESI-MS m/z calc. 254.95, found 256.0 (M+1)⁺. ¹H NMR (400 MHz, CDCl₃) δ 13.23 (br s, 1H), 7.83 (s, 1H), 2.53 (s, 3H). ¹⁹F NMR (377 MHz, CDCl₃) δ −60.81 (s, 3F).

### Step 3: 3-bromo-2-chloro-4-methyl-5-(trifluoromethyl)pyridine

3-Bromo-2-chloro-4-methyl-5-(trifluoromethyl)pyridine was prepared from 3-bromo-4-methyl-5-(trifluoromethyl)pyridin-2-ol using a procedure analogous to that found in Preparation 1, step 2. ESI-MS m/z calc. 272.92, found 273.6 (M+1)⁺. ¹H NMR (400 MHz, CDCl₃) δ 8.54 (s, 1H), 2.68-2.59 (m, 3H).

### Step 4: 3-bromo-2-(4-fluoro-2-methyl-phenoxy)-4-methyl-5-(trifluoromethyl)pyridine

3-Bromo-2-(4-fluoro-2-methyl-phenoxy)-4-methyl-5-(trifluoromethyl)pyridine was prepared from 3-bromo-2-chloro-4-methyl-5-(trifluoromethyl)pyridine and 4-fluoro-2-methyl-phenol using a procedure analogous to that found in Intermediate B-1, step 1. ESI-MS m/z calc. 362.99, found 364.1 (M+1)⁺. ¹H NMR (400 MHz, CD₃OD) δ 8.22 (s, 1H), 7.11-7.03 (m, 2H), 6.98 (td, J=8.5, 3.4 Hz, 1H), 2.63 (d, J=1.4 Hz, 3H), 2.10 (s, 3H).

### Step 5: [2-(4-fluoro-2-methyl-phenoxy)-4-methyl-5-(trifluoromethyl)-3-pyridyl]boronic acid

[2-(4-fluoro-2-methyl-phenoxy)-4-methyl-5-(trifluoromethyl)-3-pyridyl]boronic acid was prepared using a procedure analogous to that found in Intermediate B-3, step 7 using diethyl ether as the solvent. ESI-MS m/z calc. 329.09, found 330.1 (M+1)⁺. ¹H NMR (400 MHz, CD₃OD) δ 8.24 (s, 1H), 7.07-7.01 (m, 2H), 6.95 (td, J=8.4, 3.1 Hz, 1H), 2.46 (d, J=1.5 Hz, 3H), 2.11 (s, 3H).

### Intermediate B-5

### [2-(3,4-difluoro-2-methyl-phenoxy)-4-methyl-5-(trifluoromethyl)-3-pyridyl]boronic acid

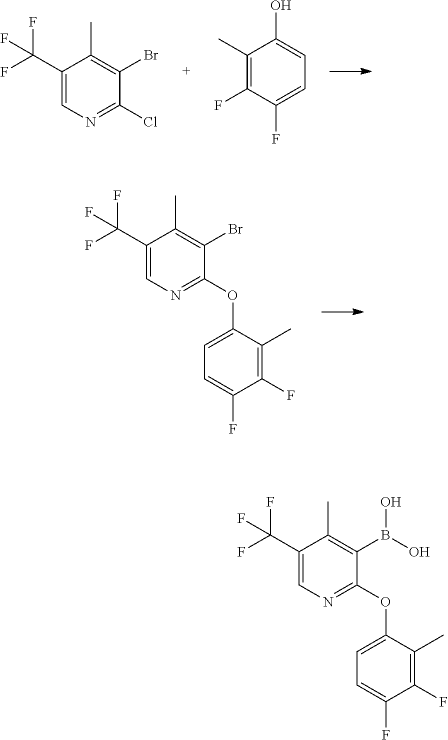

### Step 1: 3-bromo-2-(3,4-difluoro-2-methyl-phenoxy)-4-methyl-5-(trifluoromethyl)pyridine

3-Bromo-2-(3,4-difluoro-2-methyl-phenoxy)-4-methyl-5-(trifluoromethyl)pyridine was prepared from 3-bromo-2-chloro-4-methyl-5-(trifluoromethyl)pyridine and 3,4-difluoro-2-methyl-phenol using a procedure analogous to that found in Intermediate B-1, step 1. ESI-MS m/z calc. 380.98, found 382.1 (M+1)⁺. ¹H NMR (400 MHz, CDCl₃) δ 8.25 (s, 1H), 7.09 (q, J=9.2 Hz, 1H), 6.92-6.83 (m, 1H), 2.65 (s, 3H), 2.12 (d, J=2.0 Hz, 3H). ¹⁹F NMR (377 MHz, CDCl₃) δ −60.62 (s, 3F), −137.90 (d, J=21.8 Hz, 1F), −140.55 (d, J=21.8 Hz, 1F).

### Step 2: [2-(3,4-difluoro-2-methyl-phenoxy)-4-methyl-5-(trifluoromethyl)-3-pyridyl]boronic acid

[2-(3,4-Difluoro-2-methyl-phenoxy)-4-methyl-5-(trifluoromethyl)-3-pyridyl]boronic acid was prepared from 3-bromo-2-(3,4-difluoro-2-methyl-phenoxy)-4-methyl-5-(trifluoromethyl)pyridine using a procedure analogous to that found in Intermediate B-1, step 2 using trimethyl borate and diethyl ether solvent. ESI-MS m/z calc. 347.08, found 348.2 (M+1)⁺. ¹H NMR (400 MHz, CDCl₃) δ 8.34 (s, 1H), 7.15-7.02 (m, 1H), 6.90-6.79 (m, 1H), 5.50 (s, 2H), 2.71 (s, 3H), 2.11 (d, J=2.0 Hz, 3H). ¹⁹F NMR (377 MHz, CDCl₃) δ −60.66 (s, 3F), −137.80 (d, J=21.8 Hz, 1F), −140.46 (d, J=21.8 Hz, 1F).

### Intermediate B-6

### 2-(3,4-difluoro-2-methyl-phenoxy)-5-methyl-3-(4,4,5,5-tetramethyl-1,3,2-dioxaborolan-2-yl)-6-(trifluoromethyl)pyridine

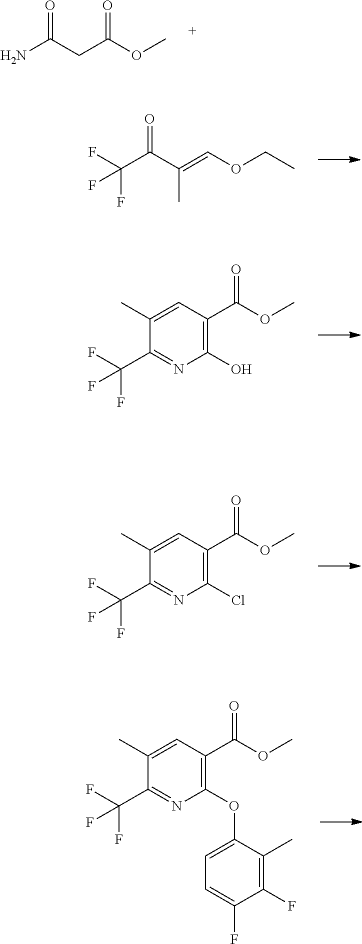

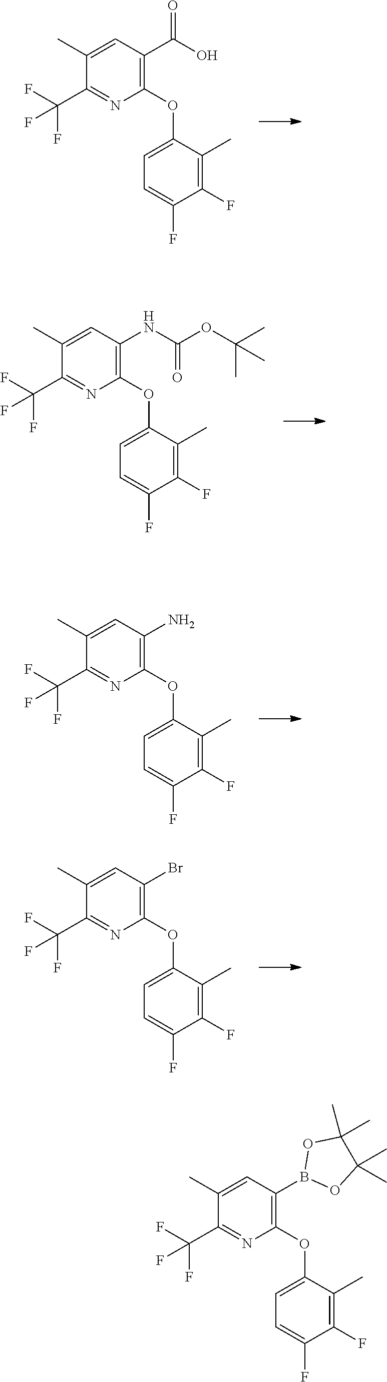 [SMILES: Cc1cc(C(=O)O)c(Oc2ccc(F)c(F)c2C)nc1C(F)(F)F ; Cc1cc(NC(=O)OC(C)(C)C)c(Oc2ccc(F)c(F)c2C)nc1C(F)(F)F ; Cc1cc(N)c(Oc2ccc(F)c(F)c2C)nc1C(F)(F)F ; Cc1cc(Br)c(Oc2ccc(F)c(F)c2C)nc1C(F)(F)F ; Cc1cc(B2OC(C)(C)C(C)(C)O2)c(Oc2ccc(F)c(F)c2C)nc1C(F)(F)F]

### Step 1: methyl 2-hydroxy-5-methyl-6-(trifluoromethyl)pyridine-3-carboxylate

To a solution of methyl 3-amino-3-oxo-propanoate (9.6 g, 82 mmol) and 4-ethoxy-1,1,1-trifluoro-3-methyl-but-3-en-2-one (15 g, 82 mmol) in methanol (120 mL) was added sodium methoxide in methanol (23 mL of 25% w/v, 106 mmol). The reaction mixture was heated at reflux for 1 h then allowed to cool to room temperature. The precipitate was removed by filtration and washed with methanol (2×250 mL). The resultant solid was suspended in ethyl acetate (500 mL) and 2 M hydrochloric acid (500 mL). The solid filtered off and the filtrate separated. The aqueous phase was re-extracted with ethyl acetate (500 mL). The combined organic extracts were washed with brine (500 mL), dried over magnesium sulfate and concentrated provided methyl 2-hydroxy-5-methyl-6-(trifluoromethyl)pyridine-3-carboxylate (11.4 g, 56%) as an off-white solid. ESI-MS m/z calc. 235.04, found 233.97 (M−1)⁻. ¹H NMR (400 MHz, CDCl₃) δ 8.17 (s, 1H), 4.03 (s, 3H), 2.45 (m, 3H).

### Step 2: methyl 2-chloro-5-methyl-6-(trifluoromethyl)pyridine-3-carboxylate

A solution of methyl 2-hydroxy-5-methyl-6-(trifluoromethyl)pyridine-3-carboxylate (10 g, 40.5 mmol) in neat phenyl dichlorophosphate (56.5 g, 40 mL, 268 mmol) was heated to 155° C. for 6 h. The reaction mixture was cooled to room temperature then quenched into a vigorously stirred mixture of ethyl acetate (1000 mL), water (250 mL) and sodium carbonate (60 g) at a rate to keep the temperature below 45° C. The mixture was then stirred vigorously for 2 h. The layers were separated. The aqueous layer was extracted with ethyl acetate (200 mL). The combined organic layers were washed with water (500 mL), brine (500 mL), dried over magnesium sulfate and concentrated. Purification by silica gel chromatography (0-20% ethyl acetate/heptane) provided methyl 2-chloro-5-methyl-6-(trifluoromethyl)pyridine-3-carboxylate (6.15 g, 56%) as a white solid. ESI-MS m/z calc. 253.01, found 253.96 (M+1)⁺. ¹H NMR (400 MHz, CDCl₃) δ 8.11 (s, 1H), 3.99 (s, 3H), 2.53 (q, J=1.7 Hz, 3H).

### Step 3: methyl 2-(3,4-difluoro-2-methyl-phenoxy)-5-methyl-6-(trifluoromethyl)pyridine-3-carboxylate

2-(3,4-Difluoro-2-methyl-phenoxy)-5-methyl-6-(trifluoromethyl)pyridine-3-carboxylic acid was prepared from methyl 2-chloro-5-methyl-6-(trifluoromethyl)pyridine-3-carboxylate and 3,4-difluoro-2-methylphenol using a procedure analogous to that found in Intermediate B-1, step 1. ESI-MS m/z calc. 361.07, found 362.06 (M+1)⁺. ¹H NMR (400 MHz, CDCl₃) δ 8.19 (s, 1H), 6.99 (q, J=9.2 Hz, 1H), 6.84 (m, 1H), 3.97 (s, 3H), 2.45 (q, J=1.8 Hz, 3H), 2.11 (d, J=2.3 Hz, 3H).

### Step 4: 2-(3,4-difluoro-2-methyl-phenoxy)-5-methyl-6-(trifluoromethyl)pyridine-3-carboxylic acid

To a mixture of methyl 2-(3,4-difluoro-2-methyl-phenoxy)-5-methyl-6-(trifluoromethyl)pyridine-3-carboxylate (30 g, 78.5 mmol) in methanol (60 mL), THF (120 mL), and water (60 mL) was added lithium hydroxide monohydrate (6.5 g, 155 mmol). The mixture was stirred at room temperature for 2 h and then the volatiles were removed under reduced pressure. The residue was acidified (˜pH 6) using 2 M HCL. The resulting solid was collected by filtration and dried to provide 2-(3,4-difluoro-2-methyl-phenoxy)-5-methyl-6-(trifluoromethyl)pyridine-3-carboxylic acid (27 g, 97%). ESI-MS m/z calc. 347.06, found 346.0 (M−1)⁻. ¹H NMR (400 MHz, CDCl₃) δ 8.39 (s, 1H), 7.07-6.99 (m, 1H), 6.86 (m, 1H), 2.48-2.44 (m, 3H), 2.15-2.08 (m, 3H).

### Step 5: tert-butyl N-[2-(3,4-difluoro-2-methyl-phenoxy)-5-methyl-6-(trifluoromethyl)-3-pyridyl]carbamate

To a solution of 2-(3,4-difluoro-2-methyl-phenoxy)-5-methyl-6-(trifluoromethyl)pyridine-3-carboxylic acid (27 g, 76 mmol) in toluene (240 mL) was added triethylamine (16 mL, 115 mmol) and DPPA (25.5 g, 20 mL, 93 mmol). The mixture was stirred at room temperature for 30 min and t-BuOH (45 mL) was added. The mixture was then heated at 110° C. for 2 h, cooled to room temperature and partitioned between ethyl acetate (400 mL) and water (200 mL). The aqueous layer was extracted with additional ethyl acetate (2×200 mL). The combined organics were washed with brine (200 mL), dried over sodium sulfate, filtered and concentrated under reduced pressure. Purification using silica gel chromatography (heptanes, followed by 10% ethyl acetate/heptanes) provided tert-butyl N-[2-(3,4-difluoro-2-methyl-phenoxy)-5-methyl-6-(trifluoromethyl)-3-pyridyl]carbamate (24 g, 74%). ESI-MS m/z calc. 418.13, found 417.05 (M−1)⁻. ¹H NMR (400 MHz, CDCl₃) δ 8.43 (s, 1H), 7.18 (s, 1H), 7.00 (q, J=9.0 Hz, 1H), 6.81 (m, 1H), 2.40 (q, J=2.0 Hz, 3H), 2.09 (d, J=2.3 Hz, 3H), 1.56 (t, J=5.0 Hz, 9H).

### Step 6: 2-(3,4-difluoro-2-methyl-phenoxy)-5-methyl-6-(trifluoromethyl)pyridin-3-amine

A solution of tert-butyl N-[2-(3,4-difluoro-2-methyl-phenoxy)-5-methyl-6-(trifluoromethyl)-3-pyridyl]carbamate (24 g, 56 mmol) in HCl in 1,4-dioxane (150 mL of 4 M, 600 mmol) was stirred at room temperature overnight. The mixture was concentrated in vacuo to provide 2-(3,4-difluoro-2-methyl-phenoxy)-5-methyl-6-(trifluoromethyl)pyridin-3-amine (21.5 g, 97%). ESI-MS m/z calc. 318.08, found 318.93 (M+1)⁺. ¹H NMR (400 MHz, CDCl₃) δ 6.98 (q, J=9.2 Hz, 1H), 6.88 (s, 1H), 6.84 (m, 1H), 2.33 (q, J=2.0 Hz, 3H), 2.11 (d, J=2.3 Hz, 3H).

### Step 7: 3-bromo-2-(3,4-difluoro-2-methyl-phenoxy)-5-methyl-6-(trifluoromethyl)pyridine

tert-Butyl nitrite (208 mg, 0.24 mL, 2.02 mmol) was added dropwise to a stirring mixture of 2-(3,4-difluoro-2-methyl-phenoxy)-5-methyl-6-(trifluoromethyl)pyridin-3-amine (300 mg, 0.823 mmol) and copper (II) bromide (420 mg, 1.88 mmol) in anhydrous acetonitrile (6 mL) under argon at 0° C. After stirring at 0° C. for 30 min, the mixture was allowed to warm to room temperature and stirred for 1 h. The mixture was diluted with water (20 mL) and extracted with ethyl acetate (3×30 mL). The combined organics were washed with brine, dried over anhydrous sodium sulfate and concentrated under reduced pressure. The crude was purified by silica gel chromatography (0-10% ethyl acetate/heptanes) yielded 3-bromo-2-(3,4-difluoro-2-methyl-phenoxy)-5-methyl-6-(trifluoromethyl)pyridine (270 mg, 83%). ¹H NMR (400 MHz, CDCl₃) δ 7.92 (s, 1H), 7.07-6.98 (m, 1H), 6.91 (m, 1H), 2.44 (q, J=1.9 Hz, 3H), 2.15 (d, J=2.4 Hz, 3H).

### Step 8: 2-(3,4-difluoro-2-methyl-phenoxy)-5-methyl-3-(4,4,5,5-tetramethyl-1,3,2-dioxaborolan-2-yl)-6-(trifluoromethyl)pyridine

2-(3,4-difluoro-2-methyl-phenoxy)-5-methyl-3-(4,4,5,5-tetramethyl-1,3,2-dioxaborolan-2-yl)-6-(trifluoromethyl)pyridine was prepared from 3-bromo-2-(3,4-difluoro-2-methyl-phenoxy)-5-methyl-6-(trifluoromethyl)pyridine using a procedure analogous to that found in Intermediate B-1, step 2 using 2-isopropoxy-4,4,5,5-tetramethyl-1,3,2-dioxaborolane. ESI-MS m/z calc. 429.15, found 346.0 (M-pinacol)⁻. ¹H NMR (400 MHz, CDCl₃) δ 8.01 (s, 1H), 6.94 (t, J=8.9 Hz, 1H), 6.87 (m, 1H), 2.40 (q, J=2.0 Hz, 3H), 2.16 (d, J=2.3 Hz, 3H), 1.34 (s, 12H).

### Intermediate B-7

### 5-(3,4-difluoro-2-methoxy-phenoxy)-4-(4,4,5,5-tetramethyl-1,3,2-dioxaborolan-2-yl)-2-(trifluoromethyl)pyridine

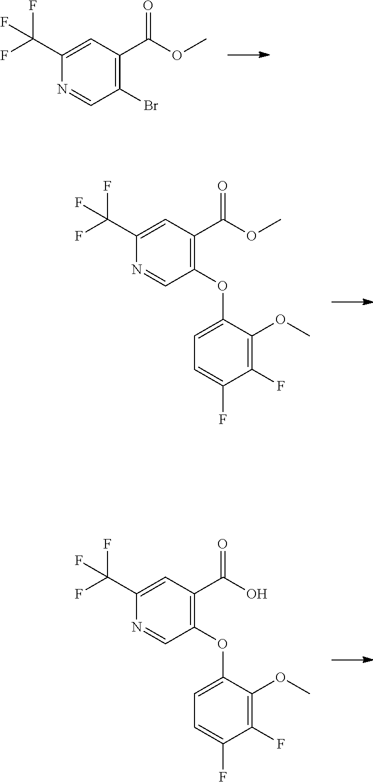 [SMILES: COC(=O)c1cc(C(F)(F)F)ncc1Br ; COC(=O)c1cc(C(F)(F)F)ncc1Oc1ccc(F)c(F)c1OC ; COc1c(Oc2cnc(C(F)(F)F)cc2C(=O)O)ccc(F)c1F]

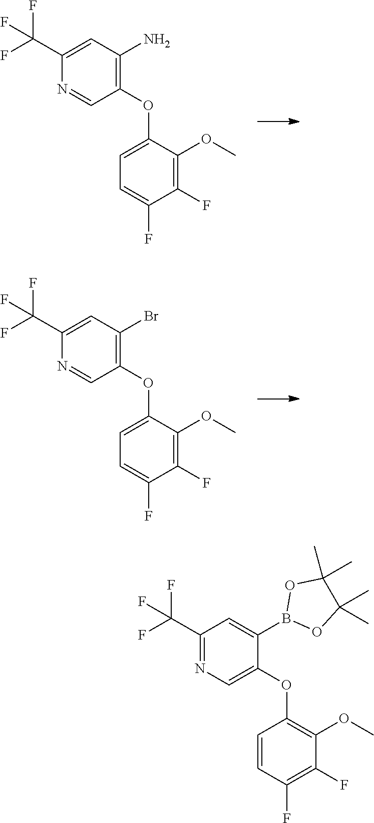 [SMILES: COc1c(Oc2cnc(C(F)(F)F)cc2N)ccc(F)c1F ; COc1c(Oc2cnc(C(F)(F)F)cc2Br)ccc(F)c1F ; COc1c(Oc2cnc(C(F)(F)F)cc2B2OC(C)(C)C(C)(C)O2)ccc(F)c1F]

### Step 1: methyl 5-(3,4-difluoro-2-methoxy-phenoxy)-2-(trifluoromethyl)pyridine-4-carboxylate

Methyl 5-(3,4-difluoro-2-methoxy-phenoxy)-2-(trifluoromethyl)pyridine-4-carboxylate was prepared from methyl 5-bromo-2-(trifluoromethyl)pyridine-4-carboxylate and 3,4-difluoro-2-methoxy-phenol using a procedure analogous to that found in Intermediate B-3, step 4 but without NN-dimethylglycine. ESI-MS m/z calc. 363.05, found 364.0 (M+1)⁺. ¹H NMR (500 MHz, CDCl₃) δ 8.24 (s, 1H), 8.12 (s, 1H), 6.95 (td, J=9.3, 7.9 Hz, 1H), 6.88 (ddd, J=9.3, 4.9, 2.2 Hz, 1H), 3.99 (s, 3H), 3.95 (d, J=1.8 Hz, 3H).

### Step 2: 5-(3,4-difluoro-2-methoxy-phenoxy)-2-(trifluoromethyl)pyridine-4-carboxylic acid

5-(3,4-Difluoro-2-methoxy-phenoxy)-2-(trifluoromethyl)pyridine-4-carboxylic acid was prepared from methyl 5-(3,4-difluoro-2-methoxy-phenoxy)-2-(trifluoromethyl)pyridine-4-carboxylate using a procedure analogous to that found in Intermediate B-6, step 4. ESI-MS m/z calc. 349.04, found 350.0 (M+1)⁺. ¹HNMR (500 MHz, DMSO-d₆) δ 14.07 (s, 1H), 8.43 (s, 1H), 8.17 (s, 1H), 7.25 (td, J=9.7, 8.4 Hz, 1H), 7.12-7.08 (m, 1H), 3.88 (d, J=1.2 Hz, 3H).

### Step 3: 5-(3,4-difluoro-2-methoxy-phenoxy)-2-(trifluoromethyl)pyridin-4-amine

5-(3,4-Difluoro-2-methoxy-phenoxy)-2-(trifluoromethyl)pyridin-4-amine was prepared from 5-(3,4-difluoro-2-methoxy-phenoxy)-2-(trifluoromethyl)pyridine-4-carboxylic acid using a procedure analogous to that found in Intermediate B-6, step 5 and step 6. ESI-MS m/z calc. 320.06, found 321.1 (M+1)⁺; 319.0 (M−1)⁻. ¹H NMR (400 MHz, CDCl₃) δ 7.91 (s, 1H), 7.06 (s, 1H), 6.90 (td, J=9.3, 8.0 Hz, 1H), 6.80 (ddd, J=9.3, 4.9, 2.3 Hz, 1H), 4.72 (s, 2H), 3.97 (d, J=1.8 Hz, 3H).

### Step 4: 4-bromo-5-(3,4-difluoro-2-methoxy-phenoxy)-2-(trifluoromethyl)pyridine

4-Bromo-5-(3,4-difluoro-2-methoxy-phenoxy)-2-(trifluoromethyl)pyridine was prepared from 5-(3,4-difluoro-2-methoxy-phenoxy)-2-(trifluoromethyl)pyridin-4-amine using a procedure analogous to that found in Intermediate B-3, step 6. ESI-MS m/z cale. 382.96, found 384.2 (M+1)⁺. ¹H NMR (400 MHz, CDCl₃) δ 8.02 (s, 1H), 7.97 (s, 1H), 7.02-6.84 (m, 2H), 3.97 (d, J=1.9 Hz, 3H).

### Step 5: 5-(3,4-difluoro-2-methoxy-phenoxy)-4-(4,4,5,5-tetramethyl-1,3,2-dioxaborolan-2-yl)-2-(trifluoromethyl)pyridine

5-(3,4-Difluoro-2-methoxy-phenoxy)-4-(4,4,5,5-tetramethyl-1,3,2-dioxaborolan-2-yl)-2-(trifluoromethyl)pyridine was prepared using a procedure analogous to that found in Intermediate B-22, step 4 using PdCl₂(PhP₃)₂ catalyst. ESI-MS m/z calc. 431.13, found 350.1 (M-pinacol)⁺.

### Intermediate B-8

### [2-(3,4-difluoro-2-methyl-phenoxy)-5-(trifluoromethyl)-3-pyridyl]boronic acid

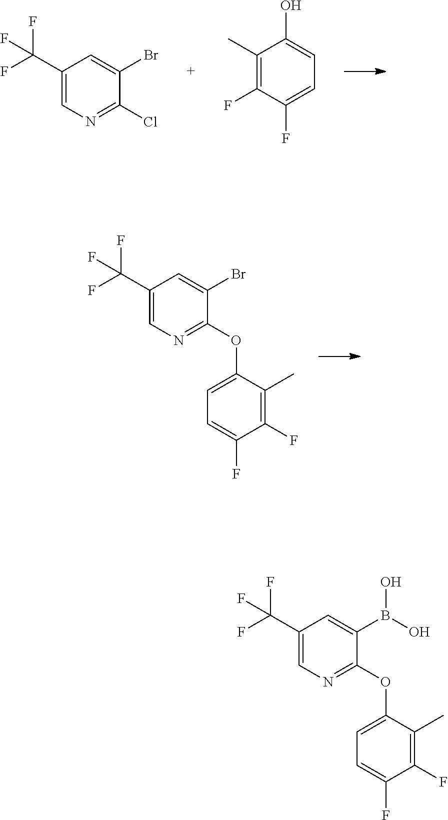

### Step 1: 3-bromo-2-(3,4-difluoro-2-methyl-phenoxy)-5-(trifluoromethyl)pyridine

3-Bromo-2-(3,4-difluoro-2-methyl-phenoxy)-5-(trifluoromethyl)pyridine was prepared from 3-bromo-2-chloro-5-(trifluoromethyl)pyridine and 3,4-difluoro-2-methyl-phenol using a procedure analogous to that found in Intermediate B-1, step 1. ESI-MS m/z calc. 366.96, found 368.0 (M+1)⁺. ¹H NMR (400 MHz, DMSO-d₆) δ 8.74-8.67 (m, 1H), 8.57-8.44 (m, 1H), 7.39 (q, J=9.4 Hz, 1H), 7.19-7.05 (m, 1H), 2.04 (d, J=2.1 Hz, 3H).

### Step 2: [2-(3,4-difluoro-2-methyl-phenoxy)-5-(trifluoromethyl)-3-pyridyl]boronic acid

[2-(3,4-Difluoro-2-methyl-phenoxy)-5-(trifluoromethyl)-3-pyridyl]boronic acid was prepared from 3-bromo-2-(3,4-difluoro-2-methyl-phenoxy)-5-(trifluoromethyl)pyridine using a procedure analogous to that found in Intermediate B-1, step 2. ESI-MS m/z calc. 333.06, found 334.0 (M+1)⁺. ¹H NMR (400 MHz, DMSO-d₆) δ 8.54 (s, 2H), 8.51-8.45 (m, 1H), 8.28-8.23 (m, 1H), 7.33 (q, J=9.4 Hz, 1H), 7.09-7.01 (m, 1H), 2.10-2.01 (m, 3H).

### Intermediate B-9

### 5-[2-methoxy-4-(trifluoromethoxy)phenoxy]-3-methyl-4-(4,4,5,5-tetramethyl-1,3,2-dioxaborolan-2-yl)-2-(trifluoromethyl)pyridine

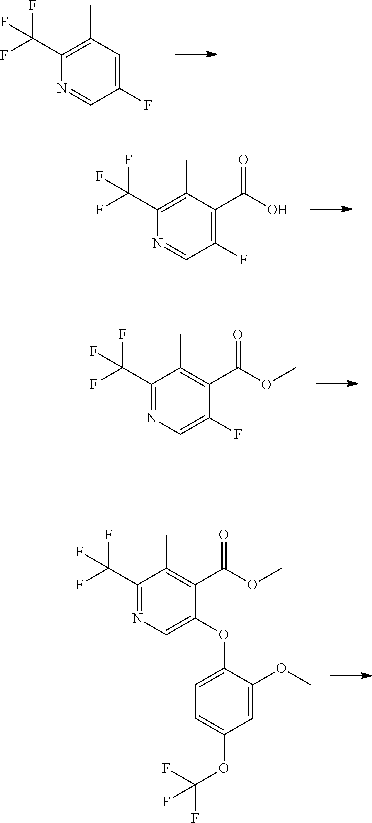 [SMILES: Cc1cc(F)cnc1C(F)(F)F ; Cc1c(C(F)(F)F)ncc(F)c1C(=O)O ; COC(=O)c1c(F)cnc(C(F)(F)F)c1C ; COC(=O)c1c(Oc2ccc(OC(F)(F)F)cc2OC)cnc(C(F)(F)F)c1C]

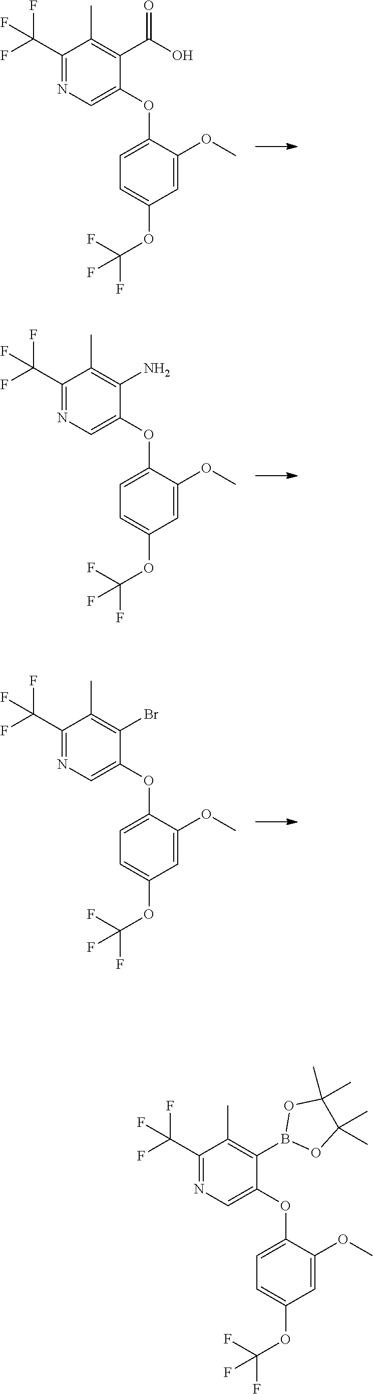 [SMILES: COc1cc(OC(F)(F)F)ccc1Oc1cnc(C(F)(F)F)c(C)c1C(=O)O ; COc1cc(OC(F)(F)F)ccc1Oc1cnc(C(F)(F)F)c(C)c1N ; COc1cc(OC(F)(F)F)ccc1Oc1cnc(C(F)(F)F)c(C)c1Br ; COc1cc(OC(F)(F)F)ccc1Oc1cnc(C(F)(F)F)c(C)c1B1OC(C)(C)C(C)(C)O1]

### Step 1: 5-fluoro-3-methyl-2-(trifluoromethyl)pyridine-4-carboxylic acid

A solution of n-BuLi (62 mL of 2.5 M in hexane, 155 mmol) was added dropwise to a solution of diisopropylamine (16.2 g, 22.5 mL, 161 mmol) in THF (190 mL) at −30° C. and the reaction mixture was slowly warmed up to 0° C. over 30 min. The reaction mixture was cooled to −78° C. and a solution of 5-fluoro-3-methyl-2-(trifluoromethyl)pyridine (23.87 g, 122.6 mmol) in THF (50 mL) was added dropwise over 25 minutes. After 1 h, solid carbon dioxide (55 g, 1.3 mol) was added in one portion (exotherm=−74 to −60° C.). After 10 min, the reaction mixture was slowly warmed up to room temperature. After 1.5 hours, heptanes (75 mL), TBME (100 mL), aqueous 1.5 M sodium hydroxide (200 mL) and water (100 mL) were added and the biphasic mixture was filtered through Celite®. The organic layer was separated and extracted with water (150 mL). The aqueous layers were combined, washed with TBME (150 mL), acidified with a 3.0 M aqueous solution of hydrochloric acid (200 mL) and extracted with additional TBME (2×200 mL). The combined organic extracts were washed with a 15% aqueous solution of sodium chloride (100 mL), dried over sodium sulfate, filtered, and concentrated to a weight of approximately 42.6 g. This mixture was slowly cooled to 0° C. over 30 min then treated with heptanes (50 mL) and the mixture stirred at 0° C. for 15 min. The solid was collected by filtration, rinsed with heptanes (50 mL), and dried under vacuum to provide 5-fluoro-3-methyl-2-(trifluoromethyl)pyridine-4-carboxylic acid (15.59 g). The mother liquor was concentrated to dryness under vacuum then resuspended in TBME (9 mL) and heated at 55° C. for 10 min. Heptanes (20 mL) was added over 25 minutes and the mixture slowly cooled to room temperature overnight. The solid was collected by filtration, rinsed with heptanes (25 mL), and dried under vacuum to provide additional 5-fluoro-3-methyl-2-(trifluoromethyl)pyridine-4-carboxylic acid (3.97 g). Both crops were combined to provide 5-fluoro-3-methyl-2-(trifluoromethyl)pyridine-4-carboxylic acid (19.56 g, 70%). ESI-MS m/z calc. 223.03, found 222.1 (M−1)⁻. ¹H NMR (400 MHz, DMSO-d₆) δ 14.74 (br. s., 1H), 8.73 (s, 1H), 2.47-2.43 (m, 3H). ¹⁹F NMR (377 MHz, DMSO-d6) δ −62.99 (s, 1F), −125.50 (s, 1F).

### Step 2: methyl 5-fluoro-3-methyl-2-(trifluoromethyl)pyridine-4-carboxylate

To a stirring mixture of 5-fluoro-3-methyl-2-(trifluoromethyl)pyridine-4-carboxylic acid (8.20 g, 36.8 mmol) in methanol (75 mL) at 0° C. was added dropwise thionyl chloride (4.9 g, 3 mL, 41 mmol). The reaction mixture was heated to 70° C. and stirred for 24 h. The reaction mixture was cooled to room temperature and partitioned between water and dichloromethane. The organic layer was washed with saturated aqueous sodium bicarbonate (2×) and brine, dried over sodium sulfate and then concentrated in vacuo. Purification by silica gel chromatography (0-20% ethyl acetate/heptane) provided methyl 5-fluoro-3-methyl-2-(trifluoromethyl)pyridine-4-carboxylate (1.085 g, 8%) as a clear, colorless oil. ESI-MS m/z calc. 237.04, found 238.03 (M+1)⁺. ¹H NMR (400 MHz, CDCl₃) δ 8.45 (s, 1H), 4.00 (s, 3H), 2.50-2.48 (m, 3H). ¹⁹F NMR (376 MHz, CDCl₃) δ −64.4 (s, 3F), −124.2 (s, 1F).

### Step 3: methyl 5-[2-methoxy-4-(trifluoromethoxy)phenoxy]-3-methyl-2-(trifluoromethyl)pyridine-4-carboxylate

Methyl 5-[2-methoxy-4-(trifluoromethoxy)phenoxy]-3-methyl-2-(trifluoromethyl)pyridine-4-carboxylate was prepared from methyl 5-fluoro-3-methyl-2-(trifluoromethyl)pyridine-4-carboxylate and 2-methoxy-4-(trifluoromethoxy)phenol using a procedure analogous to that found in Intermediate B-1, step 1 and using toluene as the solvent. ESI-MS m/z calc. 425.07, found 426.04 (M+1)⁺. ¹H NMR (400 MHz, CDCl₃) δ 7.86 (s, 1H), 7.13 (d, 1H), 6.87-6.83 (m, 2H), 3.97 (s, 3H), 3.79 (s, 3H), 2.44-2.44 (m, 3H). ¹⁹F NMR (376 MHz, CDCl₃) δ −57.9-−58.0 (m, 3F), −64.0-−64.0 (m, 3F).

### Step 4: 5-[2-methoxy-4-(trifluoromethoxy)phenoxy]-3-methyl-2-(trifluoromethyl)pyridine-4-carboxylic acid

5-[2-Methoxy-4-(trifluoromethoxy)phenoxy]-3-methyl-2-(trifluoromethyl)pyridine-4-carboxylic acid was prepared from methyl 5-[2-methoxy-4-(trifluoromethoxy)phenoxy]-3-methyl-2-(trifluoromethyl)pyridine-4-carboxylate using a procedure analogous to that found in Intermediate B-6, step 4. ESI-MS m/z calc. 411.05, found 411.98 (M+1)⁺.

### Step 5: 5-[2-methoxy-4-(trifluoromethoxy)phenoxy]-3-methyl-2-(trifluoromethyl)pyridin-4-amine

5-[2-Methoxy-4-(trifluoromethoxy)phenoxy]-3-methyl-2-(trifluoromethyl)pyridin-4-amine was prepared from 5-[2-methoxy-4-(trifluoromethoxy)phenoxy]-3-methyl-2-(trifluoromethyl)pyridine-4-carboxylic acid using a procedure analogous to that found in Intermediate B-6, steps 5 and 6. ESI-MS m/z calc. 382.08, found 383.02 (M+1)⁺. ¹H NMR (400 MHz, CDCl₃) δ 7.69 (s, 1H), 7.22 (dd, J=8.7, 7.3 Hz, 1H), 7.02-6.99 (m, 1H), 6.86-6.78 (m, 3H), 3.83 (s, 3H), 2.28 (d, J=1.4 Hz, 3H). ¹⁹F NMR (376 MHz, CDCl₃) δ −57.9-58.0 (m, 3F), −63.3 (d, J=18.8 Hz, 3F).

### Step 6: 4-bromo-5-[2-methoxy-4-(trifluoromethoxy)phenoxy]-3-methyl-2-(trifluoromethyl)pyridine

4-Bromo-5-[2-methoxy-4-(trifluoromethoxy)phenoxy]-3-methyl-2-(trifluoromethyl)pyridine was prepared from 5-[2-methoxy-4-(trifluoromethoxy)phenoxy]-3-methyl-2-(trifluoromethyl)pyridin-4-amine using a procedure analogous to that found in Intermediate B-3, step 6. ESI-MS m/z calc. 444.98, found 445.89 (M+1)⁺.

### Step 7: 5-[2-methoxy-4-(trifluoromethoxy)phenoxy]-3-methyl-4-(4,4,5,5-tetramethyl-1,3,2-dioxaborolan-2-yl)-2-(trifluoromethyl)pyridine

To a stirring mixture of 4-bromo-5-[2-methoxy-4-(trifluoromethoxy)phenoxy]-3-methyl-2-(trifluoromethyl)pyridine (200 mg, 0.448 mmol) in 2-MeTHF (2 mL) under argon was added isopropylmagnesium chloride lithium chloride (0.68 mL of 1.3 M in THF, 0.88 mmol) dropwise at 0° C. The reaction mixture was warmed to room temperature and stirred for 30 min, then 2-isopropoxy-4,4,5,5-tetramethyl-1,3,2-dioxaborolane (163 mg, 0.19 mL, 0.82 mmol) was added dropwise and stirred at 50° C. for 1 h. The mixture was quenched with a saturated solution of ammonium chloride and extracted with DCM. The organic extract was dried over sodium sulfate, filtered and concentrated to provide 5-[2-methoxy-4-(trifluoromethoxy)phenoxy]-3-methyl-4-(4,4,5,5-tetramethyl-1,3,2-dioxaborolan-2-yl)-2-(trifluoromethyl)pyridine (220 mg, 95%) as a pale yellow solid. ESI-MS m/z calc. 493.15, found 494.06 (M+1)⁺. ¹H NMR (400 MHz, CDCl₃) δ 7.82 (s, 1H), 7.02 (d, J=8.2 Hz, 1H), 6.84-6.79 (m, 2H), 3.80 (s, 3H), 2.52 (d, J=1.8 Hz, 3H), 1.34 (s, 12H). ¹⁹F NMR (376 MHz, CDCl₃) δ −58.0 (s, 3F), −64.0 (d, J=1.6 Hz, 3F).

### Intermediate B-10

### 2-[2-(3,4-difluoro-2-methoxy-phenoxy)-5-fluoro-4-(trifluoromethyl)phenyl]-4,4,5,5-tetramethyl-1,3,2-dioxaborolane

### Step 1: 1,2-difluoro-4-[4-fluoro-2-nitro-5-(trifluoromethyl)phenoxy]-3-methoxy-benzene

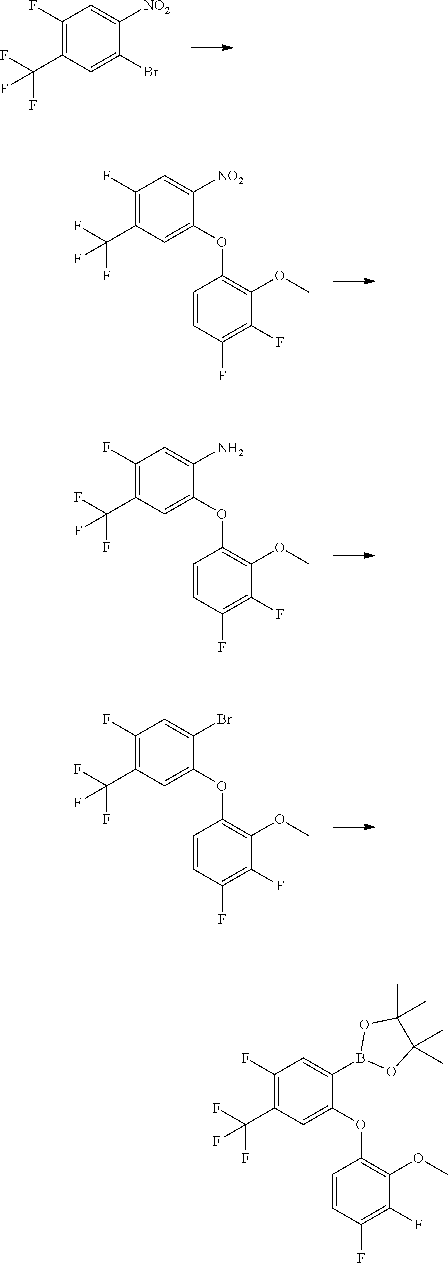 [SMILES: O=[N+]([O-])c1cc(F)c(C(F)(F)F)cc1Br ; COc1c(Oc2cc(C(F)(F)F)c(F)cc2[N+](=O)[O-])ccc(F)c1F ; COc1c(Oc2cc(C(F)(F)F)c(F)cc2N)ccc(F)c1F ; COc1c(Oc2cc(C(F)(F)F)c(F)cc2Br)ccc(F)c1F ; COc1c(Oc2cc(C(F)(F)F)c(F)cc2B2OC(C)(C)C(C)(C)O2)ccc(F)c1F]

A solution of 1-bromo-4-fluoro-2-nitro-5-(trifluoromethyl)benzene (4.526 g, 15.72 mmol), 3,4-difluoro-2-methoxy-phenol (2.589 g, 16.17 mmol) and cesium carbonate (10.3 g, 31.6 mmol) in toluene (45 mL) was purged with nitrogen for 5 min and CuI (905 mg, 4.75 mmol) was added and the reaction mixture was purged with nitrogen for 5 min. The reaction mixture was heated to 110° C. for 20 h. The mixture was diluted with ethyl acetate (100 mL) and washed with water (150 mL). The aqueous layer was extracted with additional ethyl acetate (2×100 mL), and the combined organic extracts were dried over magnesium sulfate, filtered and concentrated in vacuo. Purification by silica gel chromatography (0-10% ethyl acetate/heptane) provided 1,2-difluoro-4-[4-fluoro-2-nitro-5-(trifluoromethyl)phenoxy]-3-methoxy-benzene (2.899 g, 39%).

### Step 2: 2-(3,4-difluoro-2-methoxy-phenoxy)-5-fluoro-4-(trifluoromethyl)aniline

A solution of 1,2-difluoro-4-[4-fluoro-2-nitro-5-(trifluoromethyl)phenoxy]-3-methoxy-benzene (13.70 g, 37.31 mmol) in methanol (130 mL) was stirred with 5% Pd/C (1.6 g) under an atmosphere of hydrogen for 16 h. The mixture was filtered and concentrated to provide 2-(3,4-difluoro-2-methoxy-phenoxy)-5-fluoro-4-(trifluoromethyl)aniline (12.10 g, 91%). ¹H NMR (400 MHz, CDCl₃) δ 6.94-6.81 (m, 2H), 6.70 (ddd, J=9.3, 4.9, 2.4 Hz, 1H), 6.59 (dt, J=11.4, 0.8 Hz, 1H), 3.97 (d, J=1.6 Hz, 3H). ESI-MS m/z calc. 337.05, found 336.1 (M−1)⁻.

### Step 3: 1-bromo-2-(3,4-difluoro-2-methoxy-phenoxy)-5-fluoro-4-(trifluoromethyl)benzene

To a vial containing 2-(3,4-difluoro-2-methoxy-phenoxy)-5-fluoro-4-(trifluoromethyl)aniline (1.29 g, 3.83 mmol), copper (I) bromide (678 mg, 4.726 mmol) and lithium bromide (1.034 g, 11.91 mmol) in acetonitrile (25 mL) was added tert-butyl nitrite (680 μL, 5.72 mmol). The reaction mixture was heated to 105° C. and stirred for 20 min. The mixture was quenched with water (30 mL) and extracted with ethyl acetate (2×30 mL). The combined organic extracts were dried over magnesium sulfate, filtered and concentrated in vacuo. Purification by silica gel chromatography (0-5% ethyl acetate/heptane) provided 1-bromo-2-(3,4-difluoro-2-methoxy-phenoxy)-5-fluoro-4-(trifluoromethyl)benzene (1.102 g, 72%). ¹H NMR (500 MHz, DMSO-d₆) δ 8.14 (dd, J=9.9, 0.8 Hz, 1H), 7.31 (d, J=6.4 Hz, 1H), 7.20 (ddd, J=10.1, 9.4, 8.5 Hz, 1H), 6.90 (ddd, J=9.4, 5.0, 2.4 Hz, 1H), 3.90 (d, J=1.1 Hz, 3H).

### Step 4: 2-[2-(3,4-difluoro-2-methoxy-phenoxy)-5-fluoro-4-(trifluoromethyl)phenyl]-4,4,5,5-tetramethyl-1,3,2-dioxaborolane

2-[2-(3,4-Difluoro-2-methoxy-phenoxy)-5-fluoro-4-(trifluoromethyl)phenyl]-4,4,5,5-tetramethyl-1,3,2-dioxaborolane was prepared from 1-bromo-2-(3,4-difluoro-2-methoxy-phenoxy)-5-fluoro-4-(trifluoromethyl)benzene using a procedure analogous to that found in Intermediate B-22, step 4 and using Pd(PPh₃)₂Cl₂ as the catalyst. ¹H NMR (400 MHz, CDCl₃) δ 7.61 (d, J=10.0 Hz, 1H), 7.08 (d, J=5.5 Hz, 1H), 6.81 (td, J=9.4, 8.2 Hz, 1H), 6.47 (ddd, J=9.3, 4.8, 2.4 Hz, 1H), 4.02 (d, J=1.1 Hz, 3H), 1.26 (s, 12H). ESI-MS m/z calc. 448.13, found 365.0 (M-pinacol)⁻.

### Intermediate B-11

### [2-(4,4-difluoroazepan-1-yl)-3-quinolyl]boronic acid

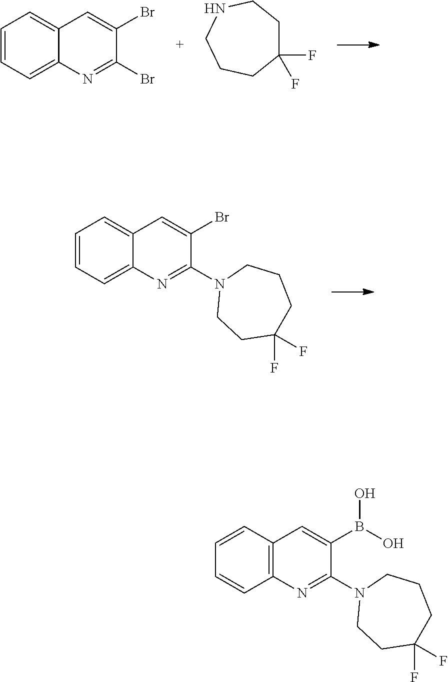 [SMILES: Brc1cc2ccccc2nc1Br ; FC1(F)CCCNCC1 ; FC1(F)CCCN(c2nc3ccccc3cc2Br)CC1 ; OB(O)c1cc2ccccc2nc1N1CCCC(F)(F)CC1]

### Step 1: 3-bromo-2-(4,4-difluoroazepan-1-yl)quinoline

A solution of 2,3-dibromoquinoline (8.16 g, 28.4 mmol) and 4,4-difluoroazepane hydrochloride (5.0 g, 29 mmol) in NMP (65 mL) was treated with potassium carbonate (7.9 g, 57.16 mmol) and heated at 80-85° C. for 2 h. Additional 4,4-difluoroazepane hydrochloride (0.5 g, 2.9 mmol) was added and the mixture stirred for another 19 h at 80-85° C. Additional 4,4-difluoroazepane hydrochloride (0.5 g, 2.9 mmol) was added and the mixture stirred for another 6 h at 80-85° C. The mixture was extracted with MTBE (250 mL)/water (500 mL) and the organic phase was washed with water (500 mL) and brine (200 mL). The aqueous phases were extracted with additional MTBE (100 mL) and the combined organic phases were dried, filtered and evaporated to provide 3-bromo-2-(4,4-difluoroazepan-1-yl)quinoline (9.48 g, 94%). ESI-MS m/z calc. 340.04, found 341.0 (M+1)⁺. ¹H NMR (400 MHz, CDCl₃) δ 8.26 (s, 1H), 7.84-7.70 (m, 1H), 7.66-7.52 (m, 2H), 7.34 (td, J=7.2, 1.2 Hz, 1H), 3.86-3.61 (m, 4H), 2.58-2.38 (m, 2H), 2.35-2.13 (m, 2H), 2.08-1.92 (m, 2H) ppm; ¹⁹F NMR (376 MHz, CDCl₃) δ −88.51.

### Step 2: [2-(4,4-difluoroazepan-1-yl)-3-quinolyl]boronic acid

n-BuLi (16 mL of 1.6 M in hexanes, 25.6 mmol) was added slowly to a stirring solution of 3-bromo-2-(4,4-difluoroazepan-1-yl)quinoline (7.42 g, 20.95 mmol) in diethyl ether (95 mL) at −78° C. under argon. The mixture was stirred at −78° C. for 1 h, then treated dropwise with a solution of trimethylborate (3.3 g, 3.5 mL, 31 mmol) in diethyl ether (35 mL). The reaction was warmed to room temperature and stirred for 18 h. The mixture was diluted with saturated aqueous ammonium chloride (200 mL) and extracted with ethyl acetate (200 mL). The organic extract was washed with brine (200 mL), dried over magnesium sulfate, filtered and concentrated. The solid was triturated with 10% ethyl acetate/heptane (10 vol.), filtered and dried to provide [2-(4,4-difluoroazepan-1-yl)-3-quinolyl]boronic acid (2.6 g, 32%) as a white solid. ESI-MS m/z calc. 306.14, found 307.15 (M+1)⁺. ¹H NMR (400 MHz, CD₃OD) δ 8.00 (s, 1H) 7.64 (t, J=7.1 Hz, 2H), 7.54-7.50 (m, 1H), 7.24-7.19 (m, 1H), 3.85-3.82 (m, 2H), 3.67 (t, J=5.5 Hz, 2H), 2.45-2.35 (m, 2H), 2.08-2.00 (m, 4H). ¹⁹F NMR (376 MHz, CD₃OD) δ −92.0-−92.1 (m, 2F).

### Intermediate B-12

### 4,4-difluoro-1-[5-methyl-3-(4,4,5,5-tetramethyl-1,3,2-dioxaborolan-2-yl)-6-(trifluoromethyl)-2-pyridyl]azepane

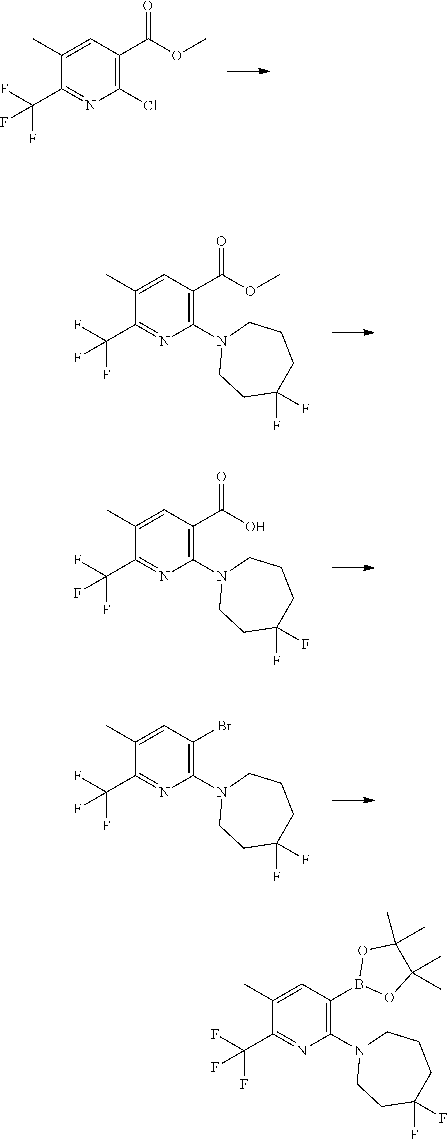 [SMILES: COC(=O)c1cc(C)c(C(F)(F)F)nc1Cl ; COC(=O)c1cc(C)c(C(F)(F)F)nc1N1CCCC(F)(F)CC1 ; Cc1cc(C(=O)O)c(N2CCCC(F)(F)CC2)nc1C(F)(F)F ; Cc1cc(Br)c(N2CCCC(F)(F)CC2)nc1C(F)(F)F ; Cc1cc(B2OC(C)(C)C(C)(C)O2)c(N2CCCC(F)(F)CC2)nc1C(F)(F)F]

### Step 1: methyl 2-(4,4-difluoroazepan-1-yl)-5-methyl-6-(trifluoromethyl)pyridine-3-carboxylate

Methyl 2-(4,4-difluoroazepan-1-yl)-5-methyl-6-(trifluoromethyl)pyridine-3-carboxylate was prepared from methyl 2-chloro-5-methyl-6-(trifluoromethyl)pyridine-3-carboxylate (Intermediate B-6, step 2) and 4,4-difluoroazepane using a procedure analogous to that found in Intermediate B-11, step 1 using cesium carbonate as the base and DMF as solvent. ESI-MS m/z calc. 352.12, found 353.15 (M+1)⁺. ¹H NMR (400 MHz, CDCl₃) δ 7.79 (s, 1H), 3.89 (s, 3H), 3.74-3.67 (m, 2H), 3.31-3.24 (m, 2H), 2.44-2.28 (m, 5H), 2.02-1.89 (m, 4H).

### Step 2: 2-(4,4-difluoroazepan-1-yl)-5-methyl-6-(trifluoromethyl)pyridine-3-carboxylic acid

2-(4,4-Difluoroazepan-1-yl)-5-methyl-6-(trifluoromethyl)pyridine-3-carboxylic acid was prepared from methyl 2-(4,4-difluoroazepan-1-yl)-5-methyl-6-(trifluoromethyl)pyridine-3-carboxylate using a procedure analogous to that found in Intermediate B-6, step 4. ESI-MS m/z calc. 338.11, found 338.99 (M+1)⁺. ¹H NMR (400 MHz, CDCl₃) δ 8.09 (s, 1H), 3.57-3.50 (m, 2H), 3.32 (t, J=6.0 Hz, 2H), 2.43-2.30 (m, 5H), 2.13-2.04 (m, 2H), 1.97-1.90 (m, 2H).

### Step 3: 1-[3-bromo-5-methyl-6-(trifluoromethyl)-2-pyridyl]-4,4-difluoro-azepane

A vial was loaded with 2-(4,4-difluoroazepan-1-yl)-5-methyl-6-(trifluoromethyl)pyridine-3-carboxylic acid (1.5 g, 4.4 mmol), potassium phosphate (941 mg, 4.43 mmol), and tetrabutylammonium tribromide (3.2 g, 6.6 mmol). The vial was capped and purged with nitrogen. Acetonitrile (22 mL) was added via syringe and the reaction was stirred at 90° C. for 1.5 h. The reaction was cooled and concentrated under reduced pressure. Purification by reverse phase chromatography (C18, 1-99% acetonitrile/5 mM HCl) provided 1-[3-bromo-5-methyl-6-(trifluoromethyl)-2-pyridyl]-4,4-difluoro-azepane (1.3 g, 79%) as a yellow oil. ESI-MS m/z calc. 372.03, found 372.9 (M+1)⁺. ¹H NMR (400 MHz, CDCl₃) δ 7.70 (s, 1H), 3.73-3.62 (m, 2H), 3.64-3.56 (m, 2H), 2.50-2.36 (m, 2H), 2.34 (s, 3H), 2.26-2.06 (m, 2H), 2.06-1.86 (m, 2H).

### Step 4: 4,4-difluoro-1-[5-methyl-3-(4,4,5,5-tetramethyl-1,3,2-dioxaborolan-2-yl)-6-(trifluoromethyl)-2-pyridyl]azepane

4,4-Difluoro-1-[5-methyl-3-(4,4,5,5-tetramethyl-1,3,2-dioxaborolan-2-yl)-6-(trifluoromethyl)-2-pyridyl]azepane was prepared from 1-[3-bromo-5-methyl-6-(trifluoromethyl)-2-pyridyl]-4,4-difluoro-azepane using a procedure analogous to that found in Intermediate B-11, step 2 and 2-isopropoxy-4,4,5,5-tetramethyl-1,3,2-dioxaborolane. ESI-MS m/z calc. 420.21, found 339.12 (M-pinacol)⁺; Mass of the corresponding boronic acid observed by LC/MS. ¹H NMR (400 MHz, CDCl₃) δ 7.66 (s, 1H), 3.73-3.70 (m, 2H), 3.52 (t, J=5.5 Hz, 2H), 2.37-2.26 (m, 5H), 2.01-1.91 (m, 4H), 1.34 (s, 12H).

The following Intermediates were made in a fashion analogous to found in Intermediate B-12, using (3S,5R)-4,4-difluoro-3,5-dimethyl-piperidine, 4,4-difluoropiperidine and 7,7-difluoro-1,2,3,3a,4,5,6,7a-octahydroisoindole in step 1, respectively.

| | | MW & | |
|---|---|---|---|
| | Compound | Found | |
| Intermediate | Name | [M + H]⁺ | NMR (shifts in ppm) |
| Intermediate | 2-[(3R,5S)-4,4-difluoro-3,5- | 434.21, | ¹H NMR (400 MHz, CDCl₃) δ 7.82 (s, |
| B-13 | dimethyl-1-piperidyl]-5- | 353.15 | 1H), 3.85-3.80 (m, 2H), 2.82 (t, J = 12.4 |
| | methyl-3-(4,4,5,5- | (M-82)+ | Hz, 2H), 2.33 (q, J = 2.0 Hz, 3H), 2.28- |
| | tetramethyl-1,3,2- | | 2.11 (m, 2H), 1.34 (s, 12H), 1.04 (d, J = |
| | dioxaborolan-2-yl)-6- | | 6.9 Hz, 6H) |
| | (trifluoromethyl)pyridine | | |
| Intermediate | 2-(4,4-difluoro-1-piperidyl)- | 406.19, | ¹H NMR (400 MHz, CDCl₃) δ 7.83 (s, |
| B-14 | 5-methyl-3-(4,4,5,5- | found | 1H), 3.53 (t, J = 5.7 Hz, 4H), 2.33 (q, J = |
| | tetramethyl-1,3,2- | 325.04 | 2.1 Hz, 3H), 2.15-2.03 (m, 4H), 1.35 (s, |
| | dioxaborolan-2-y1)-6- | (M-82)+ | 12H) |
| | (trifluoromethyl)pyridine | | |
| Intermediate | [2-(7,7-difluoro- | 364.14, | |
| B-15 | 3,3a,4,5,6,7a-hexahydro-1H- | 365.0 | |
| | isoindol-2-y1)-5-methyl-6- | | |
| | (trifluoromethyl)-3- | | |
| | pyridyl]boronic acid | | |

### Intermediate B-16

### [2-(4,4-difluoro-1-piperidyl)-5-(trifluoromethyl)-3-pyridyl]boronic acid

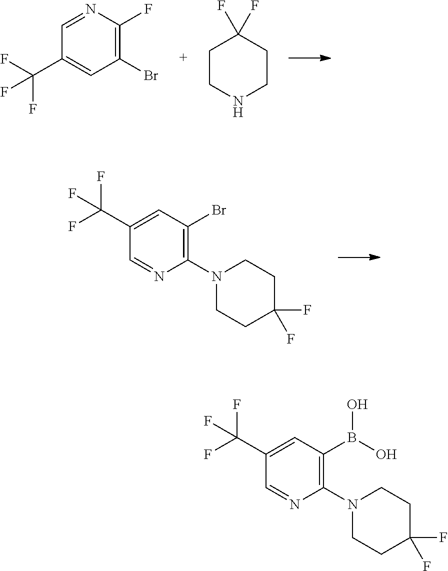 [SMILES: Fc1ncc(C(F)(F)F)cc1Br ; FC1(F)CCNCC1 ; FC1(F)CCN(c2ncc(C(F)(F)F)cc2Br)CC1 ; OB(O)c1cc(C(F)(F)F)cnc1N1CCC(F)(F)CC1]

### Step 1: 3-bromo-2-(4,4-difluoro-1-piperidyl)-5-(trifluoromethyl)pyridine

To a solution of 3-bromo-2-fluoro-5-(trifluoromethyl)pyridine (1.224 g, 5.017 mmol) in THF (6 mL) at 0° C. was added a solution of 4,4-difluoropiperidine (1.22 g, 10.1 mmol) in THF (2 ml) dropwise. The mixture was removed from the ice bath and allowed to come to room temperature over 1 h, then heated at 50° C. for 24 h. The mixture was partitioned between water and ethyl acetate. The organic layer was separated and washed with 50% saturated aqueous ammonium chloride, dried over sodium sulfate, filtered and concentrated in vacuo. Purification by silica gel chromatography (40 g silica, 10-100% ethyl acetate/hexane) provided 3-bromo-2-(4,4-difluoro-1-piperidyl)-5-(trifluoromethyl)pyridine (1.68 g, 97%). ESI-MS m/z calc. 343.99, found 345.0 (M+1)⁺. ¹H NMR (400 MHz, DMSO-d₆) δ 8.62 (dd, J=2.2, 1.1 Hz, 1H), 8.40-8.35 (m, 1H), 3.58-3.51 (m, 4H), 2.19-2.06 (m, 4H).

### Step 2: [2-(4,4-difluoro-1-piperidyl)-5-(trifluoromethyl)-3-pyridyl]boronic acid

[2-(4,4-Difluoro-1-piperidyl)-5-(trifluoromethyl)-3-pyridyl]boronic acid was prepared from 3-bromo-2-(4,4-difluoro-1-piperidyl)-5-(trifluoromethyl)pyridine using a procedure analogous to that found in Intermediate B-11, step 2 with triisopropyl borate and diethyl ether as the solvent. ESI-MS m z calc. 310.09, found 311.0 (M+1)⁺. ¹H NMR (400 MHz, DMSO-d₆) δ 8.42 (dd, J=2.6, 1.2 Hz, 1H), 7.79 (d, J=2.6 Hz, 1H), 3.66-3.55 (m, 4H), 2.16-1.98 (m, 4H).

The following Intermediate was prepared from 3-bromo-2-fluoro-6-(trifluoromethyl)pyridine and 4,4-difluoro-azepane using a procedure analogous to that found in Intermediate B-16

| | | MW & | |
|---|---|---|---|
| | Compound | Found | |
| Intermediate | Name | [M + H]⁺ | NMR (shifts in ppm) |
| Intermediate | [2-(4,4-difluoroazepan-1-yl)- | 324.11, | 1H NMR (400 MHz, DMSO-d₆) δ 7.72- |
| B-17 | 6-(trifluoromethyl)-3- | 325.2 | 7.62 (m, 1H), 7.00 (dd, J = 7.3, 1.3 Hz, |
| | pyridyl]boronic acid | | 1H), 3.71-3.60 (m, 2H), 3.54-3.46 (m, |
| | | | 3H), 2.39-2.18 (m, 2H), 2.13-1.83 (m, |
| | | | 5H). |

### Intermediate B-18

### [2-(4,4-difluoroazepan-1-yl)-6-methyl-5-(trifluoromethyl)-3-pyridyl]boronic acid

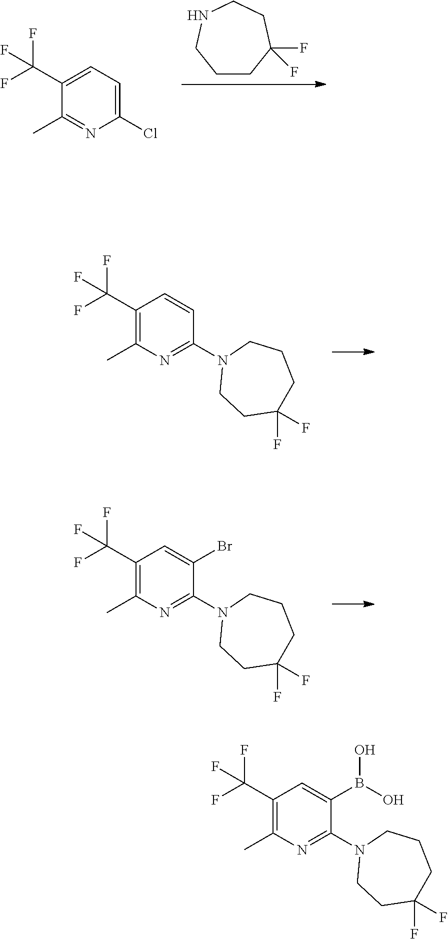 [SMILES: Cc1nc(Cl)ccc1C(F)(F)F ; FC1(F)CCCNCC1 ; Cc1nc(N2CCCC(F)(F)CC2)ccc1C(F)(F)F ; Cc1nc(N2CCCC(F)(F)CC2)c(Br)cc1C(F)(F)F ; Cc1nc(N2CCCC(F)(F)CC2)c(B(O)O)cc1C(F)(F)F]

### Step 1: 4,4-difluoro-1-[6-methyl-5-(trifluoromethyl)-2-pyridyl]azepane

4,4-Difluoro-1-[6-methyl-5-(trifluoromethyl)-2-pyridyl]azepane was prepared from 6-chloro-2-methyl-3-(trifluoromethyl)pyridine using a procedure analogous to that found in Intermediate B-11, step 1 with DMSO as solvent. ESI-MS m/z calc. 294.12, found 295.5 (M+1)⁺. ¹H NMR (400 MHz, CDCl₃) δ 7.60 (d, J=8.9 Hz, 1H), 6.30 (d, J=8.9 Hz, 1H), 3.81-3.76 (m, 2H), 3.65 (t, J=6.1 Hz, 2H), 2.54-2.49 (m, 3H), 2.30-2.16 (m, 2H), 2.11-1.94 (m, 4H).

### Step 2: 1-[3-bromo-6-methyl-5-(trifluoromethyl)-2-pyridyl]-4,4-difluoro-azepane

4,4-Difluoro-1-[6-methyl-5-(trifluoromethyl)-2-pyridyl]azepane (1.29 g, 4.38 mmol) and NBS (786 mg, 4.42 mmol) were combined in DCM (25 mL) and stirred at room temperature for 16 h. The reaction was evaporated and the resulting material purified by silica gel chromatography (0-20% ethyl acetate/hexanes) to provide 1-[3-bromo-6-methyl-5-(trifluoromethyl)-2-pyridyl]-4,4-difluoro-azepane (1.34 g, 82%) as a clear oil. ESI-MS m/z calc. 372.03, found 373.2 (M+1)⁺. ¹H NMR (400 MHz, CDCl₃) δ 7.86 (s, 1H), 3.77 (t, J=6.3 Hz, 2H), 3.74-3.69 (m, 2H), 2.50-2.46 (m, 3H), 2.46-2.33 (m, 2H), 2.19-2.06 (m, 2H), 2.01-1.93 (m, 2H).

### Step 3: [2-(4,4-difluoroazepan-1-yl)-6-methyl-5-(trifluoromethyl)-3-pyridyl]boronic acid

[2-(4,4-Difluoroazepan-1-yl)-6-methyl-5-(trifluoromethyl)-3-pyridyl]boronic acid was prepared from 1-[3-bromo-6-methyl-5-(trifluoromethyl)-2-pyridyl]-4,4-difluoro-azepane using a procedure analogous to that found in Intermediate B-11, step 2. ESI-MS m/z calc. 338.12, found 339.3 (M+1)⁺. ¹H NMR (400 MHz, CD₃OD) δ 7.62 (s, 1H), 3.80-3.75 (m, 2H), 3.54-3.48 (m, 2H), 2.48 (s, 3H), 2.36-2.21 (m, 2H), 2.00-1.92 (m, 4H).

### Intermediate B-19

### [5-chloro-2-(4,4-difluoroazepan-1-yl)-6-(trifluoromethyl)-3-pyridyl]boronic acid

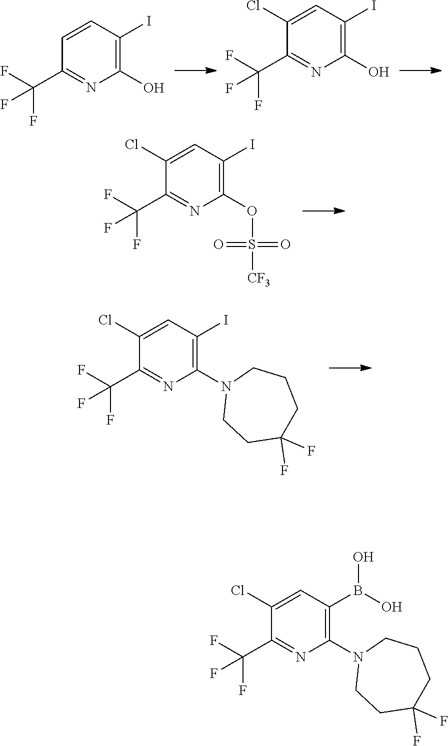

### Step 1: 5-chloro-3-iodo-6-(trifluoromethyl)pyridin-2-ol

To a solution of 3-iodo-6-(trifluoromethyl)pyridin-2-ol (15.0 g, 51.0 mmol) in DMF (75 mL) was added 1,3-dichloro-5,5-dimethylhydantoin (18.0 g, 91.4 mmol). The reaction mixture was stirred 24 h at room temperature then partitioned between ethyl acetate (500 mL) and water (200 mL). The vigorously stirred biphasic mixture was cooled to 0-10° C. and sodium thiosulfate 10% aqueous solution (200 mL) was added slowly. Sodium hydroxide 25% aqueous solution was added until neutral pH was reached (˜20 mL). The layers were separated and the aqueous layer was extracted with ethyl acetate (2×100 mL). The combined organic layers were washed with water (4×100 mL), brine (100 mL), dried over sodium sulfate, filtered and concentrated. The pH of the aqueous layer was adjusted to 3 by addition of HCl 3 N aqueous solution (˜20 mL) and was extracted with ethyl acetate (4×100 mL). The combined organic layers were washed with water (50 mL), brine (50 mL), dried over sulfate, filtered and concentrated. The combined extracted materials were purified by silica gel chromatography (0-10% methanol/DCM) to provide 5-chloro-3-iodo-6-(trifluoromethyl)pyridin-2-ol (7.37 g, 45%). ¹H NMR (400 MHz, CDCl₃) δ 9.30 (br.s, 1H), 8.20 (s, 1H). ¹⁹F NMR (377 MHz, CDCl₃) δ −65.64 (s, 3F). ESI-MS m/z calc. 322.88, found 324.0 (M+1)⁺.

### Step 2: [5-chloro-3-iodo-6-(trifluoromethyl)-2-pyridyl]trifluoromethanesulfonate

To a solution of 5-chloro-3-iodo-6-(trifluoromethyl)pyridin-2-ol (7.37 g, 22.8 mmol) in DCM (150 mL) at 0° C. was added DIPEA (8.9 g, 12 mL, 69 mmol) and trifluoromethanesulfonic anhydride (13.4 g, 8.0 mL, 48 mmol). The resulting mixture was stirred overnight at room temperature. The mixture was diluted with DCM (350 mL), cooled to 0° C. and treated with saturated aqueous sodium bicarbonate (200 mL) under vigorous stirring. The layers were separated and the aqueous layer was extracted with DCM (100 mL). The combined organic layers were washed with water (100 mL), dried over sodium sulfate, filtered and concentrated under reduced pressure. The residue was taken up in pentane (100 mL), filtered to remove insoluble solids and concentrated. Purification by silica gel chromatography (220 g silica, 0-15% ethyl acetate/heptane) provided [5-chloro-3-iodo-6-(trifluoromethyl)-2-pyridyl]trifluoromethanesulfonate (8.89 g, 86%). ESI-MS m/z calc. 454.83, found 456.0 (M+1)⁺. ¹HNMR (400 MHz, CDCl₃) δ 8.45 (s, 1H). ¹⁹F NMR (377 MHz, CDCl₃) δ −66.56 (s, 3F), −71.85 (s, 3F).

### Step 3: 1-[5-chloro-3-iodo-6-(trifluoromethyl)-2-pyridyl]-4,4-difluoro-azepane

1-[5-Chloro-3-iodo-6-(trifluoromethyl)-2-pyridyl]-4,4-difluoro-azepane was prepared from [5-chloro-3-iodo-6-(trifluoromethyl)-2-pyridyl]trifluoromethanesulfonate using a procedure analogous to that found in Intermediate B-11, step 1 with DIPEA as base and DMF as solvent. ESI-MS m/z calc. 439.96, found 441.2 (M+1)⁺. ¹H NMR (400 MHz, CDCl₃) δ 8.17 (s, 1H), 3.71-3.57 (m, 4H), 2.47-2.33 (m, 2H), 2.24-2.09 (m, 2H), 2.02-1.92 (m, 2H). ¹⁹F NMR (377 MHz, CDCl₃) δ −66.38 (s, 3F), −89.34 (quin, J=15.0 Hz, 2F).

### Step 4: [5-chloro-2-(4,4-difluoroazepan-1-yl)-6-(trifluoromethyl)-3-pyridyl]boronic acid

[5-Chloro-2-(4,4-difluoroazepan-1-yl)-6-(trifluoromethyl)-3-pyridyl]boronic acid was prepared from 1-[5-chloro-3-iodo-6-(trifluoromethyl)-2-pyridyl]-4,4-difluoro-azepane using a procedure analogous to that found in Intermediate B-11, step 2 with triisopropyl borate and THF as the solvent. ESI-MS m/z calc. 358.07, found 359.1 (M+1)⁺. ¹H NMR (400 MHz, CDCl₃) δ 7.14 (s, 1H), 5.35 (s, 2H), 3.80-3.72 (m, 2H), 3.66 (t, J=6.2 Hz, 2H), 2.31-2.17 (m, 2H), 2.12-1.94 (m, 4H). ¹⁹F NMR (377 MHz, CDCl₃) δ −65.93 (s, 3F), −91.23 (quin, J=14.3 Hz, 2F).

### Intermediate B-20

### [2-(4,4-difluoroazepan-1-yl)-5,6,7,8-tetrahydroquinolin-3-yl]boronic acid

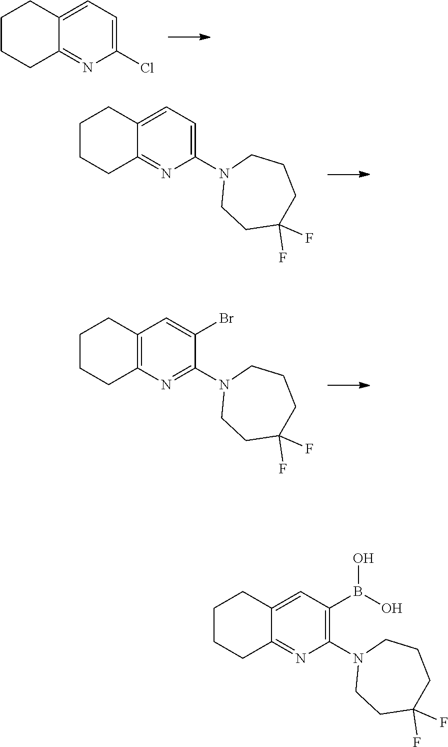

### Step 1: 2-(4,4-difluoroazepan-1-yl)-5,6,7,8-tetrahydroquinoline

2-Chloro-5,6,7,8-tetrahydroquinoline (505 mg, 3.01 mmol), 4,4-difluoroazepane hydrochloride (535 mg, 3.12 mmol), potassium carbonate (1.321 g, 9.558 mmol), dicyclohexyl-[2-(2,6-diisopropoxyphenyl)phenyl]phosphane (88.6 mg, 0.190 mmol), and diacetoxypalladium (35 mg, 0.16 mmol) were combined in DMSO (2.5 mL). The reaction mixture was flushed with nitrogen and heated at 110° C. for 24 h. The reaction was cooled, diluted with DCM (2 mL) and purified by silica gel chromatography (0-10% methanol/dichloromethane) to provide 2-(4,4-difluoroazepan-1-yl)-5,6,7,8-tetrahydroquinoline (532 mg, 66%). ESI-MS m/z calc. 266.16, found 267.4 (M+1)⁺. ¹H NMR (400 MHz, CDCl₃) δ 7.15 (d, J=8.5 Hz, 1H), 6.28 (d, J=8.5 Hz, 1H), 3.74-3.68 (m, 2H), 3.60 (t, J=6.2 Hz, 2H), 2.72 (t, J=6.4 Hz, 2H), 2.65-2.57 (m, 2H), 2.28-2.14 (m, 2H), 2.09-1.90 (m, 4H), 1.87-1.79 (m, 2H), 1.79-1.72 (m, 2H).

### Step 2: 3-bromo-2-(4,4-difluoroazepan-1-yl)-5,6,7,8-tetrahydroquinoline

3-Bromo-2-(4,4-difluoroazepan-1-yl)-5,6,7,8-tetrahydroquinoline was prepared from 2-(4,4-difluoroazepan-1-yl)-5,6,7,8-tetrahydroquinoline using a procedure analogous to that found in Intermediate B-18, step 2. ESI-MS m/z calc. 344.07, found 345.2 (M+1)⁺. ¹H NMR (400 MHz, CDCl₃) δ 7.46 (s, 1H), 3.56-3.47 (m, 4H), 2.71 (t, J=6.4 Hz, 2H), 2.64 (t, J=6.3 Hz, 2H), 2.44-2.29 (m, 2H), 2.27-2.13 (m, 2H), 1.97-1.88 (m, 2H), 1.88-1.79 (m, 2H), 1.79-1.71 (m, 2H).

### Step 3: [2-(4,4-difluoroazepan-1-yl)-5,6,7,8-tetrahydroquinolin-3-yl]boronic acid

[2-(4,4-difluoroazepan-1-yl)-5,6,7,8-tetrahydroquinolin-3-yl]boronic acid was prepared from 3-Bromo-2-(4,4-difluoroazepan-1-yl)-5,6,7,8-tetrahydroquinoline using a procedure analogous to that found in Intermediate B-11, step 2. ESI-MS m/z calc. 310.17, found 311.2 (M+1)⁺.

### Intermediate B-21

### [2-(4,4-difluoroazepan-1-yl)-6,7-dihydro-5H-cyclopenta[b]pyridin-3-yl]boronic acid

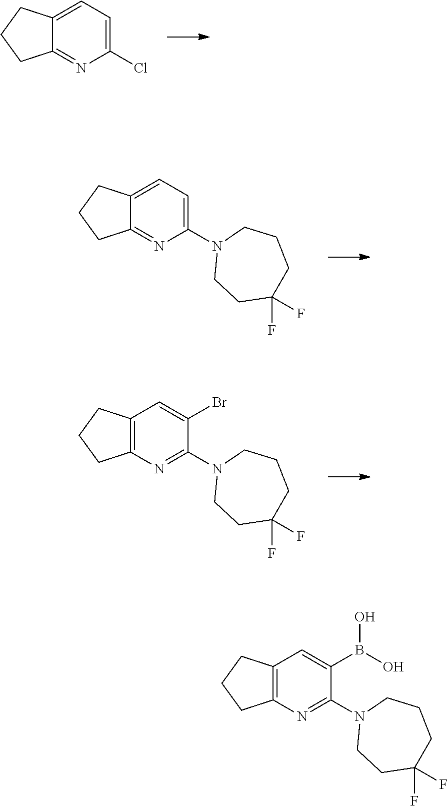 [SMILES: Clc1ccc2c(n1)CCC2 ; FC1(F)CCCN(c2ccc3c(n2)CCC3)CC1 ; FC1(F)CCCN(c2nc3c(cc2Br)CCC3)CC1 ; OB(O)c1cc2c(nc1N1CCCC(F)(F)CC1)CCC2]

### Step 1: 2-(4,4-difluoroazepan-1-yl)-6,7-dihydro-5H-cyclopenta[b]pyridine

2-(4,4-Difluoroazepan-1-yl)-6,7-dihydro-5H-cyclopenta[b]pyridine was prepared from 2-Chloro-6,7-dihydro-5H-cyclopenta[b]pyridine using a procedure analogous to that found in Intermediate B-20, step 1. ESI-MS m/z calc. 252.14, found 253.3 (M+1)⁺. ¹H NMR (400 MHz, CDCl₃) δ 7.30 (d, J=8.5 Hz, 1H), 6.24 (d, J=8.4 Hz, 1H), 3.75-3.69 (m, 2H), 3.62 (t, J=6.2 Hz, 2H), 2.85 (t, J=7.6 Hz, 2H), 2.79 (t, J=7.3 Hz, 2H), 2.29-2.17 (m, 2H), 2.11-1.92 (m, 6H).

### Step 2: 3-bromo-2-(4,4-difluoroazepan-1-yl)-6,7-dihydro-5H-cyclopenta[b]pyridine

3-Bromo-2-(4,4-difluoroazepan-1-yl)-6,7-dihydro-5H-cyclopenta[b]pyridine was prepared from 2-(4,4-difluoroazepan-1-yl)-6,7-dihydro-5H-cyclopenta[b]pyridine using a procedure analogous to that found in Intermediate B-18, step 2. ESI-MS m/z calc. 330.05, found 331.2 (M+1)⁺. ¹H NMR (400 MHz, CDCl₃) δ 7.60 (s, 1H), 3.54-3.44 (m, 4H), 2.89-2.79 (m, 4H), 2.45-2.30 (m, 2H), 2.30-2.17 (m, 2H), 2.11 (p, J=7.6 Hz, 2H), 1.98-1.88 (m, 2H).

### Step 3: [2-(4,4-difluoroazepan-1-yl)-6,7-dihydro-5H-cyclopenta[b]pyridin-3-yl]boronic acid

[2-(4,4-Difluoroazepan-1-yl)-6,7-dihydro-5H-cyclopenta[b]pyridin-3-yl]boronic acid was prepared from -bromo-2-(4,4-difluoroazepan-1-yl)-6,7-dihydro-5H-cyclopenta[b]pyridine using a procedure analogous to that found in Intermediate B-11, step 2. ESI-MS m/z calc. 296.15, found 297.1 (M+1)⁺.

### Intermediate B-22

### [2-(4,4-difluoroazepan-1-yl)-7-fluoro-3-quinolyl]boronic acid

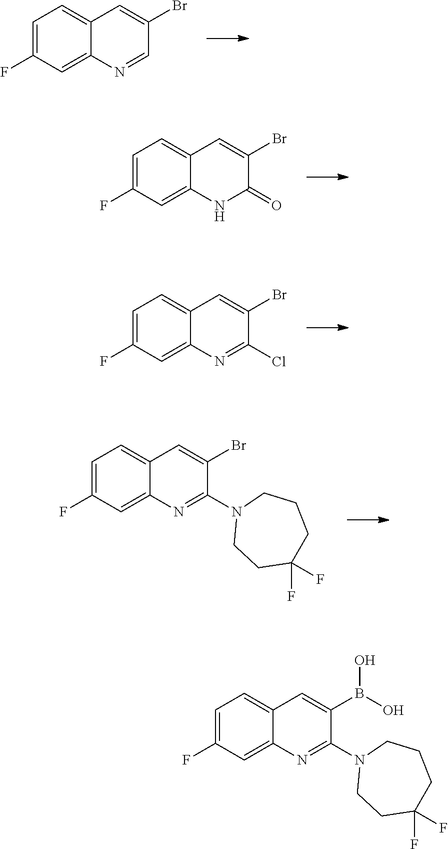 [SMILES: Fc1ccc2cc(Br)cnc2c1 ; O=c1[nH]c2cc(F)ccc2cc1Br ; Fc1ccc2cc(Br)c(Cl)nc2c1 ; Fc1ccc2cc(Br)c(N3CCCC(F)(F)CC3)nc2c1 ; OB(O)c1cc2ccc(F)cc2nc1N1CCCC(F)(F)CC1]

### Step 1: 3-bromo-7-fluoro-1H-quinolin-2-one

Methyltrioxorhenium (70 mg, 0.20 mmol) was added to a solution of hydrogen peroxide (23 mL of 30% w/v in water, 202.85 mmol) in THF (18 mL) and the mixture was stirred for 15 min. 3-Bromo-7-fluoro-quinoline (4.5 g, 20 mmol) was added and the mixture stirred at room temperature for 20 h. The mixture was diluted with ethyl acetate (150 mL), cooled to 5° C. and quenched using aqueous 10% sodium thiosulfate (150 mL). The organic layer was separated and the aqueous layer was extracted with ethyl acetate (2×150 mL). The combined organic extracts were washed with saturated aqueous sodium bicarbonate (150 mL) and brine (150 mL), dried over sodium sulfate, filtered and concentrated under reduced pressure. The crude product was adsorbed on silica gel and purified by silica gel chromatography (120 g silica, 0-80% ethyl acetate/heptane) to afford 3-bromo-7-fluoro-1H-quinolin-2-one (4.19 g, 87%) as a tan solid. ESI-MS m/z calc. 240.95, found 242.0 (M+1)⁺. ¹H NMR (400 MHz, CDCl₃) δ 8.65 (s, 1H), 8.35 (dd, J=9.5, 2.2 Hz, 1H), 7.91 (s, 1H), 7.83 (dd, J=9.0, 5.4 Hz, 1H), 7.46 (d, J=1.2 Hz, 1H). 19F NMR (377 MHz, CDCl₃) δ −104.95-105.05 (m, 1F).

### Step 2: 3-bromo-2-chloro-7-fluoro-quinoline

3-Bromo-2-chloro-7-fluoro-quinoline was prepared from 3-bromo-7-fluoro-1H-quinolin-2-one using a procedure analogous to that found in Preparation 1, step 2. ESI-MS m/z calc. 258.92, found 260.0 (M+1)⁺. ¹H NMR (400 MHz, CDCl₃) δ 8.44 (s, 1H), 7.80-7.75 (m, 1H), 7.66 (dd, J=9.7, 2.3 Hz, 1H), 7.39 (td, J=8.6, 2.4 Hz, 1H). 19F NMR (377 MHz, CDCl₃) δ−106.35-106.44 (m, 1F).

### Step 3: 3-bromo-2-(4,4-difluoroazepan-1-yl)-7-fluoro-quinoline

A solution of 3-bromo-2-chloro-7-fluoro-quinoline (4.14 g, 15.5 mmol), 4,4-difluoroazepane hydrochloride (4.0 g, 23 mmol), cesium carbonate (12.7 g, 39.0 mmol), copper iodide (300 mg, 1.58 mmol) and N,N,N′,N″-tetramethylethane-1,2-diamine (186 mg, 0.24 mL, 1.6 mmol) in NMP (50 mL) was stirred at 105° C. overnight. Once cooled to room temperature, the reaction mixture was diluted with water (50 mL) and extracted using ethyl acetate (3×20 mL). The organic layers were combined, washed with brine (20 mL), dried over sodium sulfate and concentrated under reduced pressure. The residue was purified by silica gel chromatography (0-10% of ethyl acetate/heptanes), followed by reverse phase chromatography (C18, 5-95% acetonitrile/0.1% formic acid) to provide 3-bromo-2-(4,4-difluoroazepan-1-yl)-7-fluoro-quinoline (2.3 g, 37%) as a light yellow oil. ESI-MS m/z calc. 358.03, found 359.0 (M+1)⁺. ¹H NMR (400 MHz, CDCl₃) δ 8.23 (s, 1H), 7.57 (dd, J=8.8, 6.1 Hz, 1H), 7.38 (dd, J=10.5, 2.4 Hz, 1H), 7.11 (td, J=8.6, 2.6 Hz, 1H), 3.80-3.68 (m, 4H), 2.55-2.42 (m, 2H), 2.29-2.17 (m, 2H), 2.05-1.98 (m, 2H). ¹⁹F NMR (377 MHz, CDCl₃) δ −88.99 (quin, J=15.0 Hz, 2F), −109.50-−109.60 (m, 1F).

### Step 4: [2-(4,4-difluoroazepan-1-yl)-7-fluoro-3-quinolyl]boronic acid

A suspension of 3-bromo-2-(4,4-difluoroazepan-1-yl)-7-fluoro-quinoline (200 mg, 0.501 mmol), 4,4,5,5-tetramethyl-2-(4,4,5,5-tetramethyl-1,3,2-dioxaborolan-2-yl)-1,3,2-dioxaborolane (385 mg, 1.52 mmol) and potassium acetate (150 mg, 1.53 mmol) in 1,4-dioxane (3 mL) was purged with nitrogen for 5 min, then Pd(dppf)Cl₂.DCM (42 mg, 0.051 mmol) was added and the mixture was purged with nitrogen for 5 min. The tube was sealed and the mixture was stirred at 80° C. for 20 hours. The mixture was cooled to room temperature, filtered through Celite®, and the filter rinsed with ethyl acetate (20 mL). The filtrate was concentrated under reduced pressure to afford [2-(4,4-difluoroazepan-1-yl)-7-fluoro-3-quinolyl]boronic acid (162 mg, 100%). ESI-MS m/z calc. 324.11, found 325.2 (M+1)⁺.

### Intermediate B-23

### [2-(4,4-difluoroazepan-1-yl)-6-fluoro-3-quinolyl]boronic acid

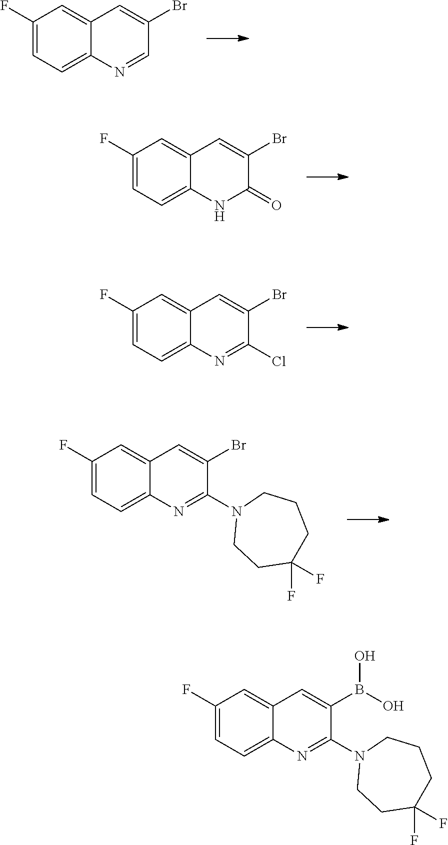 [SMILES: Fc1ccc2ncc(Br)cc2c1 ; O=c1[nH]c2ccc(F)cc2cc1Br ; Fc1ccc2nc(Cl)c(Br)cc2c1 ; Fc1ccc2nc(N3CCCC(F)(F)CC3)c(Br)cc2c1 ; OB(O)c1cc2cc(F)ccc2nc1N1CCCC(F)(F)CC1]

### Step 1: 3-bromo-6-fluoro-1H-quinolin-2-one

3-Bromo-6-fluoro-1H-quinolin-2-one was prepared from 3-bromo-6-fluoro-quinoline using a procedure analogous to that found in Intermediate B-22, step 1. ESI-MS m/z calc. 240.95, found 242.0 (M+1)⁺. ¹H NMR (400 MHz, DMSO-d₆) δ 8.87 (s, 1H), 8.53 (dd, J=9.4, 5.3 Hz, 1H), 8.26 (s, 1H), 7.89 (dd, J=9.2, 2.8 Hz, 1H), 7.80-7.70 (m, 1H). ¹⁹F NMR (377 MHz, DMSO-d₆) δ −109.88 (s, 1F).

### Step 2: 3-bromo-2-chloro-6-fluoro-quinoline

3-Bromo-2-chloro-6-fluoro-quinoline was prepared from 3-bromo-6-fluoro-1H-quinolin-2-one using a procedure analogous to that found in Preparation 1, step 2. ESI-MS m/z calc. 258.92, found 259.9 (M+1)⁺. ¹H NMR (400 MHz, DMSO-d₆) δ 8.94 (s, 1H), 8.07 (dd, J=9.2, 5.3 Hz, 1H), 7.89-7.74 (m, 2H). ¹⁹F NMR (377 MHz, DMSO-d₆) δ −110.94 (s, 1F).

### Step 3: 3-bromo-2-(4,4-difluoroazepan-1-yl)-6-fluoro-quinoline

3-Bromo-2-(4,4-difluoroazepan-1-yl)-6-fluoro-quinoline was prepared from 3-bromo-2-(4,4-difluoroazepan-1-yl)-6-fluoro-quinoline and 4,4-difluoroazepane hydrochloride using a procedure analogous to that found in Intermediate B-22, step 3. ESI-MS m/z calc. 358.03, found 359.0 (M+1)⁺. ¹H NMR (400 MHz, DMSO-d₆) δ 8.59 (s, 1H), 7.75 (dd, J=9.3, 5.4 Hz, 1H), 7.66-7.49 (m, 2H), 3.71-3.60 (m, 4H), 2.49-2.38 (m, 2H), 2.31-2.13 (m, 2H), 1.98-1.87 (m, 2H). ¹⁹F NMR (377 MHz, DMSO-d₆) δ −86.16 (s, 2F), −116.86 (s, 1F).

### Step 4: [2-(4,4-difluoroazepan-1-yl)-6-fluoro-3-quinolyl]boronic acid

[2-(4,4-Difluoroazepan-1-yl)-6-fluoro-3-quinolyl]boronic acid was prepared from 3-bromo-2-(4,4-difluoroazepan-1-yl)-6-fluoro-quinoline using a procedure analogous to that found in Intermediate B-22, step 4 using 4,4,5,5-tetramethyl-2-(4,4,5,5-tetramethyl-1,3,2-dioxaborolan-2-yl)-1,3,2-dioxaborolane and 1,4-dioxane as the organic solvent. ESI-MS m/z calc. 324.11, found 325.1 (M+1)⁺.

### Intermediate B-24

### 4-(4,4,5,5-tetramethyl-1,3,2-dioxaborolan-2-yl)-2-(trifluoromethyl)-5-[4-(trifluoromethyl)cyclohexyl]pyridine

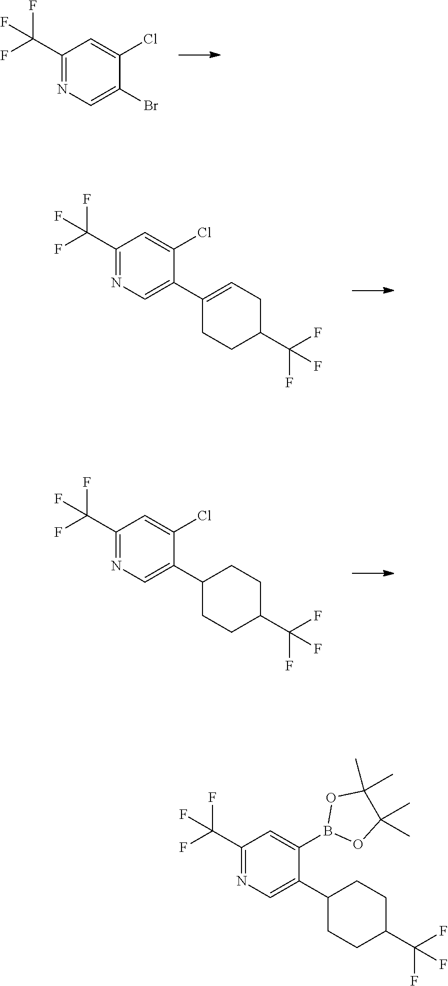 [SMILES: FC(F)(F)c1cc(Cl)c(Br)cn1 ; FC(F)(F)c1cc(Cl)c(C2=CCC(C(F)(F)F)CC2)cn1 ; FC(F)(F)c1cc(Cl)c(C2CCC(C(F)(F)F)CC2)cn1 ; CC1(C)OB(c2cc(C(F)(F)F)ncc2C2CCC(C(F)(F)F)CC2)OC1(C)C]

### Step 1: 4-chloro-2-(trifluoromethyl)-5-[4-(trifluoromethyl)cyclohexen-1-yl]pyridine

A vial was charged with 5-bromo-4-chloro-2-(trifluoromethyl)pyridine (617 mg, 2.37 mmol), 4,4,5,5-tetramethyl-2-[4-(trifluoromethyl)cyclohexen-1-yl]-1,3,2-dioxaborolane (654 mg, 2.37 mmol), Pd(dppf)Cl₂ (223 mg, 0.304 mmol) and potassium carbonate (1.11 g, 8.03 mmol). 1,4-dioxane (14 mL) and water (2 mL) were added and the mixture degassed for 5 min. The vial was sealed and mixture heated at 110° C. under nitrogen atmosphere for 4 h. The mixture was cooled to room temperature, diluted with dichloromethane (20 mL) and washed with saturated aqueous ammonium chloride and brine. The organic layer was dried over magnesium sulfate, filtered and concentrated under reduced pressure. Purification by silica gel chromatography (0-50% ethyl acetate/heptanes over 20 min) provided 4-chloro-2-(trifluoromethyl)-5-[4-(trifluoromethyl)cyclohexen-1-yl]pyridine (616 mg, 79%). ESI-MS m/z calc. 329.04, found 330.1 (M+1)⁺. ¹H NMR (400 MHz, DMSO-d₆) δ 8.61 (s, 1H), 8.14 (s, 1H), 5.93-5.88 (m, 1H), 2.75-2.59 (m, 1H), 2.58-2.43 (m, 2H), 2.43-2.30 (m, 1H), 2.30-2.18 (m, 1H), 2.11-2.03 (m, 1H), 1.70-1.55 (m, 1H). ¹⁹F NMR (376 MHz, DMSO-d₆) δ −66.35, −72.13.

### Step 2: 4-chloro-2-(trifluoromethyl)-5-[4-(trifluoromethyl)cyclohexyl]pyridine

A mixture of 4-chloro-2-(trifluoromethyl)-5-[4-(trifluoromethyl)cyclohexen-1-yl]pyridine (300 mg, 0.910 mmol) and 5% rhodium on alumina (350 mg, 0.170 mmol) in isopropanol (12 mL) was stirred under hydrogen atmosphere for 3 h. The mixture was filtered and the solvent was removed under reduced pressure. Purification by silica gel chromatography (0-50% ethyl acetate/heptanes over 15 min) provided 4-chloro-2-(trifluoromethyl)-5-[4-(trifluoromethyl)cyclohexyl]pyridine (178.1 mg, 59%) as a mixture of cis- and trans-isomers. ESI-MS m/z calc. 331.06, found 332.1 (M+1)⁺. ¹H NMR (400 MHz, DMSO-d₆) δ 8.76 (s, 0.25H), 8.73 (s, 0.75H), 8.10 (s, 1H), 3.15-2.99 (m, 1H), 2.68-2.54 (m, 0.75H), 2.46-2.35 (m, 0.25H), 2.05-1.90 (m, 2.5H), 1.89-1.64 (m, 5H), 1.47 (qd, J=12.8, 3.4 Hz, 0.5H). ¹⁹F NMR (376 MHz, DMSO-d₆) δ −66.03 (major), −66.34 (minor) overlaps with −66.35 (major), −72.31 (minor).

### Step 3: 4-(4,4,5,5-tetramethyl-1,3,2-dioxaborolan-2-yl)-2-(trifluoromethyl)-5-[4-(trifluoromethyl)cyclohexyl]pyridine

4-(4,4,5,5-Tetramethyl-1,3,2-dioxaborolan-2-yl)-2-(trifluoromethyl)-5-[4-(trifluoromethyl)cyclohexyl]pyridine was prepared from 4-chloro-2-(trifluoromethyl)-5-[4-(trifluoromethyl)cyclohexyl]pyridine using a procedure analogous to that found in Intermediate B-22, step 4 and 1,4-dioxane as the organic solvent. The product was isolated as a mixture of cis- and trans-isomers. ESI-MS m/z calc. 423.18, found 424.153 (M+1)⁺. ¹H NMR (400 MHz, DMSO-d₆) δ 8.74 (s, 0.25H), 8.70 (s, 0.75H), 7.86 (s, 0.75H) overlaps with 7.85 (s, 0.25H), 3.31-3.11 (m, 1H), 2.65-2.54 (m, 1H), 2.07-1.87 (m, 2H), 1.75 (m, 5H), 1.45-1.36 (m, 1H), 1.17 (s, 12H). ¹⁹F NMR (376 MHz, DMSO-d₆) δ −65.92 (major), −66.28 (major minor), −72.34 (minor).

### Intermediate B-25

### 5-(4,4,5,5-tetramethyl-1,3,2-dioxaborolan-2-yl)-2-(trifluoromethyl)-4-[4-(trifluoromethyl)cyclohexyl]pyridine (3:1 cis/trans mixture)

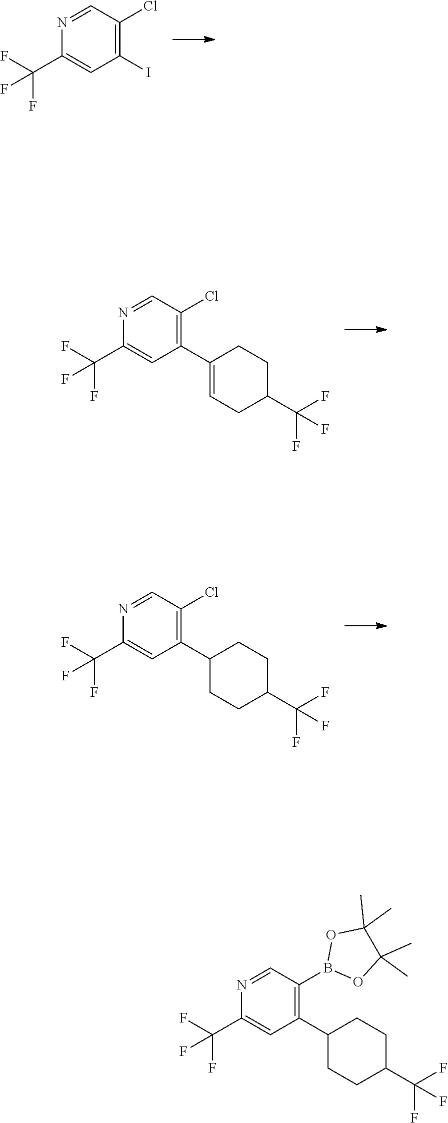 [SMILES: FC(F)(F)c1cc(I)c(Cl)cn1 ; FC(F)(F)c1cc(C2=CCC(C(F)(F)F)CC2)c(Cl)cn1 ; FC(F)(F)c1cc(C2CCC(C(F)(F)F)CC2)c(Cl)cn1 ; CC1(C)OB(c2cnc(C(F)(F)F)cc2C2CCC(C(F)(F)F)CC2)OC1(C)C]

### Step 1: 5-chloro-2-(trifluoromethyl)-4-[4-(trifluoromethyl)cyclohexen-1-yl]pyridine

5-Chloro-2-(trifluoromethyl)-4-[4-(trifluoromethyl)cyclohexen-1-yl]pyridine was prepared from 4,4,5,5-tetramethyl-2-[4-(trifluoromethyl)cyclohexen-1-yl]-1,3,2-dioxaborolane and 5-chloro-4-iodo-2-(trifluoromethyl)pyridine using a cross-coupling procedure analogous to that found in Intermediate B-24, step 1. ESI-MS m/z calc. 329.04, found 330.1 (M+1)⁺.

### Step 2: 5-chloro-2-(trifluoromethyl)-4-[4-(trifluoromethyl)cyclohexyl]pyridine (Cis/Trans Mixture)

5-Chloro-2-(trifluoromethyl)-4-[4-(trifluoromethyl)cyclohexyl]pyridine (3:1 cis/trans mixture) was prepared from 5-chloro-2-(trifluoromethyl)-4-[4-(trifluoromethyl)cyclohexen-1-yl]pyridine using an alkene reduction procedure analogous to that found in Intermediate B-24, step 2. ESI-MS m/z calc. 331.06, found 332.0 (M+1)⁺.

### Step 3: 5-(4,4,5,5-tetramethyl-1,3,2-dioxaborolan-2-yl)-2-(trifluoromethyl)-4-[4-(trifluoromethyl)cyclohexyl]pyridine (3:1 cis/trans mixture)

5-(4,4,5,5-Tetramethyl-1,3,2-dioxaborolan-2-yl)-2-(trifluoromethyl)-4-[4-(trifluoromethyl)cyclohexyl]pyridine (3:1 cis/trans mixture) was prepared from 5-chloro-2-(trifluoromethyl)-4-[4-(trifluoromethyl)cyclohexyl]pyridine from step 2 and 4,4,5,5-tetramethyl-2-(4,4,5,5-tetramethyl-1,3,2-dioxaborolan-2-yl)-1,3,2-dioxaborolane using a procedure analogous to that found in Intermediate B-22, step 4. ESI-MS m/z calc. 423.18, found 424.1 (M+1)⁺.

### Intermediate B-26

### [6-tert-butyl-4-[4-(trifluoromethyl)cyclohexyl]-3-pyridyl]boronic acid

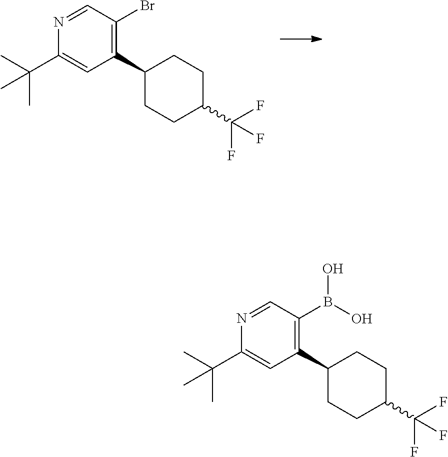

A vial was charged with 5-bromo-2-tert-butyl-4-[4-(trifluoromethyl)cyclohexyl]pyridine (3:1 mixture of cis/trans isomers) (140 mg, 0.384 mmol), bis(pinacol)diboron (151 mg, 0.595 mmol), potassium acetate (121 mg, 1.23 mmol) and CataCXium A Pd G3 (20 mg, 0.026 mmol). The vial was sealed and flushed with nitrogen. N,N-dimethylacetamide (1 mL) was then added under nitrogen atmosphere and the reaction was stirred at 80° C. for 4 h. The reaction was cooled to room temperature, filtered and purified by reverse phase HPLC (C18, 1-99% acetonitrile/5 mM HCl) to provide [6-tert-butyl-4-[4-(trifluoromethyl)cyclohexyl]-3-pyridyl]boronic acid as mixture of cis trans isomers. ESI-MS m/z calc. 329.18, found 330.1 (M+1)⁺.

### Intermediate B-27

### 2-(3,3-difluorocyclobutyl)-5-(4,4,5,5-tetramethyl-1,3,2-dioxaborolan-2-yl)-4-(4-(trifluoromethyl)cyclohexyl)pyridine (cis isomer)

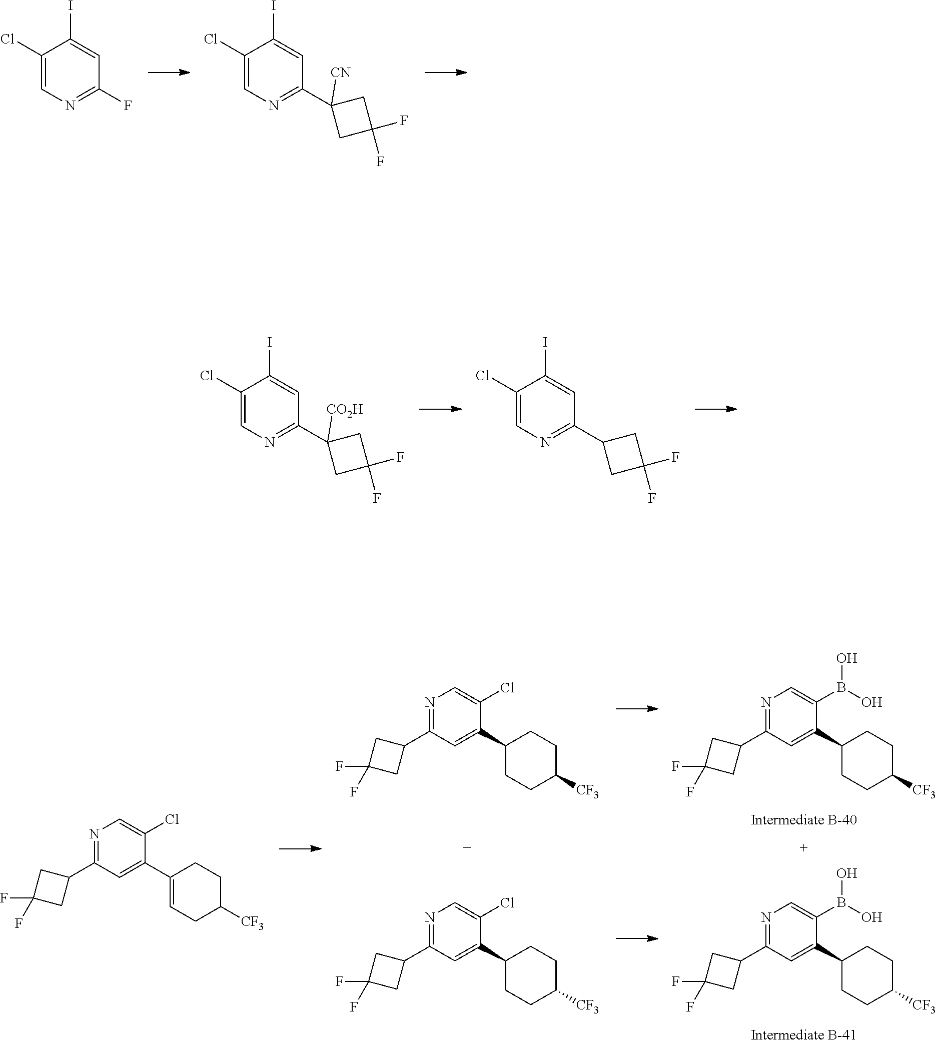

### Step 1: Synthesis of 1-(5-chloro-4-iodopyridin-2-yl)-3,3-difluorocyclobutane-1-carbonitrile

To a solution of 3,3-difluorocyclobutanecarbonitrile (5.0 g, 42.7 mmol) in toluene (200 mL) at 0° C. was added LiHMDS (50 mL of 1 M in toluene, 50 mmol) and the mixture stirred for 10 min. A solution of 5-chloro-2-fluoro-4-iodo-pyridine (11.37 g, 42.10 mmol) in toluene (50 mL) was added. The reaction mixture was allowed to warm to room temperature and stirred overnight. The reaction was quenched with saturated ammonium chloride solution (200 mL) and the mixture extracted with ethyl acetate (400 mL). The organic extract was washed with brine (150 mL), then dried over magnesium sulfate, filtered and concentrated. Purification on silica (0-2.9% ethyl acetate/heptane) provided 1-(5-chloro-4-iodopyridin-2-yl)-3,3-difluoro-cyclobutanecarbonitrile (19% yield) as a white solid. ¹H NMR (400 MHz, CDCl₃) δ 8.55 (s, 1H), 8.14 (s, 1H), 3.54-3.42 (m, 2H), 3.36-3.26 (m, 2H).

### Step 2: 1-(5-chloro-4-iodo-2-pyridyl)-3,3-difluoro-cyclobutanecarboxylic acid

To 1-(5-chloro-4-iodo-2-pyridyl)-3,3-difluoro-cyclobutanecarbonitrile (8.07 g, 22.3 mmol) in acetic acid (90 mL) was added water (45 mL) and sulfuric acid (45 mL of 95% w/v, 844 mmol). The resulting mixture was heated at 85° C. for 6 h. On cooling the reaction mixture was diluted with water (800 mL) and extracted with ethyl acetate (2×800 mL). The combined extracts were washed with brine (250 mL), dried over magnesium sulfate, filtered and concentrated under reduced pressure provided a brown gum 1-(5-chloro-4-iodo-2-pyridyl)-3,3-difluoro-cyclobutanecarboxylic acid (8.26 g, 97%). ESI-MS m/z calc. 372.92, found 327.86 (M-CO₂H)—.

### Step 3: 5-chloro-2-(3,3-difluorocyclobutyl)-4-iodopyridine

A solution of 1-(5-chloro-4-iodo-2-pyridyl)-3,3-difluoro-cyclobutanecarboxylic acid (8.26 g, 21.3 mmol) in toluene (100 mL) was heated at 100° C. overnight. The reaction mixture was concentrated under reduced pressure to provide 5-chloro-2-(3,3-difluorocyclobutyl)-4-iodopyridine (7 g, 98%) as a cream colored solid. ¹H NMR (400 MHz, CDCl₃) δ 8.50 (s, 1H), 7.68 (s, 1H), 3.41-3.31 (m, 1H), 2.94-2.84 (m, 4H).

### Step 4: 5-chloro-2-(3,3-difluorocyclobutyl)-4-(4-(trifluoromethyl)cyclohex-1-en-1-yl)pyridine

5-Chloro-2-(3,3-difluorocyclobutyl)-4-[4-(trifluoromethyl)cyclohex-1-en-1-yl]pyridine was prepared from 5-chloro-2-(3,3-difluorocyclobutyl)-4-iodo-pyridine using a procedure analogous to that found in Intermediate B-24, step 1. ESI-MS m/z calc. 351.08, found 352.09 (M+1)⁺. ¹H NMR (400 MHz, CDCl₃) δ 8.52 (s, 1H), 6.96 (s, 1H), 5.78 (s, 1H), 3.44-3.40 (m, 1H), 2.96-2.84 (m, 4H), 2.49-2.29 (m, 5H), 2.14 (d, J=12.8 Hz, 1H), 1.71 (qd, J=12.1, 5.4 Hz, 1H).

### Step 5: 5-chloro-2-(3,3-difluorocyclobutyl)-4-((1S,4S)-4-(trifluoromethyl)cyclohexyl)pyridine (cis isomer) and 5-chloro-2-(3,3-difluorocyclobutyl)-4-((1R,4R)-4-(trifluoromethyl)cyclohexyl)pyridine (trans isomer)

5-Chloro-2-(3,3-difluorocyclobutyl)-4-(4-(trifluoromethyl)cyclohexyl)pyridine (cis and trans isomers) were prepared from 5-chloro-2-(3,3-difluorocyclobutyl)-4-[4-(trifluoromethyl)cyclohexen-1-yl]pyridine using a procedure analogous to that found in Intermediate B-24, step 2. Purification using silica gel chromatography (0-3.5% ethyl acetate/heptane over 41 min) provided product-containing fractions which were combined and concentrated giving 5-chloro-2-(3,3-difluorocyclobutyl)-4-(4-(trifluoromethyl)cyclohexyl)pyridine (cis isomer, 1.12 g, 19%) as a white solid. ¹H NMR (400 MHz, CDCl₃) δ 8.49 (s, 1H), 7.02 (s, 1H), 3.44-3.35 (m, 1H), 3.47-3.30 (m, 1H), 2.95-2.86 (m, 4H), 2.48-2.32 (m, 1H), 2.20-2.03 (m, 2H), 1.80-1.65 (m, 6H). ESI-MS m/z calc. 353.10, found 354.16 (M+1)⁺; Retention time: 3.23 min and 5-chloro-2-(3,3-difluorocyclobutyl)-4-(4-(trifluoromethyl)cyclohexyl)pyridine (trans isomer, 0.69 g, 11%) as a white solid. ESI-MS m/z calc. 353.10, found 354.14 (M+1)⁺; Retention time: 3.2 min. ¹H NMR (400 MHz, CDCl₃) δ 8.50-8.48 (m, 1H), 6.99 (s, 1H), 3.43-3.34 (m, 1H), 3.03-2.83 (m, 5H), 2.18-2.00 (m, 5H), 1.58-1.37 (m, 4H). Retention times for the cis and trans product isomers were determined using the following conditions: Waters UPLC, BEH C18 column, 2.1×50 mm, 2.5 m particle, 2-95% acetonitrile in water (0.1% NH₃ modifier), 4.6 min run, 0.8 ml/min, 40° C.

### Step 6: 2-(3,3-difluorocyclobutyl)-5-(4,4,5,5-tetramethyl-1,3,2-dioxaborolan-2-yl)-4-(4-(trifluoromethyl)cyclohexyl)pyridine (cis isomer)

2-(3,3-Difluorocyclobutyl)-5-(4,4,5,5-tetramethyl-1,3,2-dioxaborolan-2-yl)-4-(4-(trifluoromethyl)cyclohexyl)pyridine (cis isomer) was prepared from 5-chloro-2-(3,3-difluorocyclobutyl)-4-(4-(trifluoromethyl)cyclohexyl)pyridine (cis isomer) using a procedure analogous to that found in Intermediate B-22, step 4 using SPhos Pd G3 as catalyst. Product was isolated as a ˜1:1 mixture of boronic acid and pinacol ester and used without further purification. ESI-MS m/z calc. 445.22, found 446.3 (M+1)⁺; Retention time: 3.40 min (pinacol ester), and ESI-MS m/z calc. 363.14, found 364.2 (M+1); Retention time: 2.03 min (boronic acid). Retention times were determined using the following conditions: Waters UPLC, BEH C18 column (2.1×50 mm, 2.5 m), 2-95% acetonitrile in water (0.1% NH₃ modifier), 4.6 min run, 0.8 mL/min, 40° C.

### Intermediate B-28

### 2-(3,3-difluorocyclobutyl)-5-(4,4,5,5-tetramethyl-1,3,2-dioxaborolan-2-yl)-4-(4-(trifluoromethyl)cyclohexyl)pyridine (trans isomer)

2-(3,3-Difluorocyclobutyl)-5-(4,4,5,5-tetramethyl-1,3,2-dioxaborolan-2-yl)-4-(4-(trifluoromethyl)cyclohexyl)pyridine (trans isomer) was prepared from 5-chloro-2-(3,3-difluorocyclobutyl)-4-(4-(trifluoromethyl)cyclohexyl)pyridine (trans isomer isolated from Intermediate B-27, step 6) using a procedure analogous to that found in Intermediate B-22, step 4 using SPhos Pd G3 as catalyst. NMR analysis shows isolated product as a ˜2:3 mixture of boronic acid and pinacol ester, and used without further purification. ESI-MS m/z calc. 445.22, found 364.14 (M-pinacol)⁺, only boronic acid observed by LC/MS.

### Intermediate B-29

### 2-tert-butyl-4-(4,4-difluorocyclohexyl)-5-(4,4,5,5-tetramethyl-1,3,2-dioxaborolan-2-yl)pyrimidine

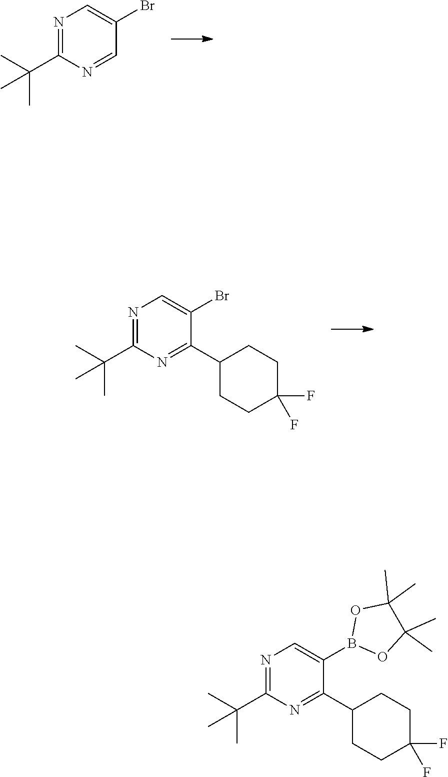 [SMILES: CC(C)(C)c1ncc(Br)cn1 ; CC(C)(C)c1ncc(Br)c(C2CCC(F)(F)CC2)n1 ; CC(C)(C)c1ncc(B2OC(C)(C)C(C)(C)O2)c(C2CCC(F)(F)CC2)n1]

### Step 1: 5-bromo-2-tert-butyl-4-(4,4-difluorocyclohexyl)pyrimidine

5-Bromo-2-tert-butyl-pyrimidine (2.48 g, 11.0 mmol), 4,4-difluorocyclohexanecarboxylic acid (2.762 g, 16.49 mmol), AgNO₃ (3.752 g, 22.08 mmol), and ammonium persulfate (6.39 g, 27.4 mmol) were combined in a flask, then dissolved in a mixture of acetonitrile (50 mL) and water (50 mL) and heated at 60° C. for 3.5 h. Additional 4,4-difluorocyclohexanecarboxylic acid (279 mg, 1.67 mmol), AgNO₃ (386 mg, 2.27 mmol) and ammonium persulfate (657 mg, 2.82 mmol) were added and stirring continued for 1 h. The reaction was cooled to room temperature and the acetonitrile removed under reduced pressure. The mixture was partitioned between brine and ethyl acetate and the layers separated. The organic phase was extracted with additional ethyl acetate (3×), then the combined organic phases were washed with brine, dried over magnesium sulfate, filtered, and concentrated under reduced pressure. Purification by silica gel chromatography (40 g, 0-50% ethyl acetate/hexanes) provided 5-bromo-2-tert-butyl-4-(4,4-difluorocyclohexyl)pyrimidine (3.44 g, 85%) as a clear, colorless oil. ESI-MS m/z calc. 332.07, found 333.1 (M+1)⁺. ¹H NMR (400 MHz, DMSO-d₆) δ 8.84 (s, 1H), 3.28-3.19 (m, 1H), 2.18-2.02 (m, 2H), 1.98-1.79 (m, 6H), 1.33 (s, 9H). ¹⁹F NMR (376 MHz, DMSO-d₆) δ −89.69, −90.31, −99.04, −99.66.

### Step 2: 2-tert-butyl-4-(4,4-difluorocyclohexyl)-5-(4,4,5,5-tetramethyl-1,3,2-dioxaborolan-2-yl)pyrimidine

2-tert-Butyl-4-(4,4-difluorocyclohexyl)-5-(4,4,5,5-tetramethyl-1,3,2-dioxaborolan-2-yl)pyrimidine was prepared from 5-bromo-2-tert-butyl-4-(4,4-difluorocyclohexyl)pyrimidine and 2-isopropoxy-4,4,5,5-tetramethyl-1,3,2-dioxaborolane using a procedure analogous to that found in Intermediate B-1, step 2. ESI-MS m/z calc. 380.25, found 381.27 (M+1)⁺.

### Intermediate B-30

### [4-(4,4-difluorocyclohexyl)-2-(trifluoromethyl)pyrimidin-5-yl]boronic acid

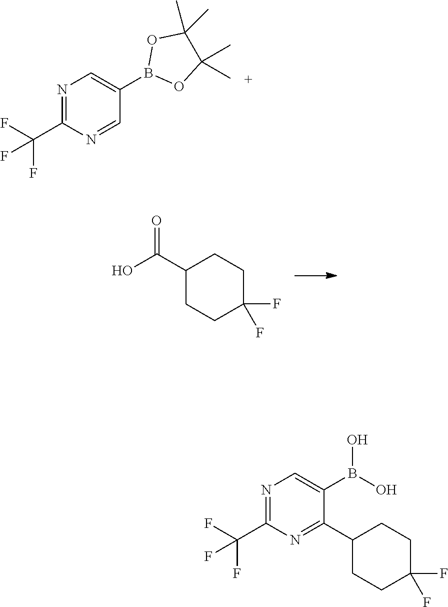 [SMILES: CC1(C)OB(c2cnc(C(F)(F)F)nc2)OC1(C)C ; O=C(O)C1CCC(F)(F)CC1 ; OB(O)c1cnc(C(F)(F)F)nc1C1CCC(F)(F)CC1]

[4-(4,4-Difluorocyclohexyl)-2-(trifluoromethyl)pyrimidin-5-yl]boronic acid was prepared from 5-(4,4,5,5-tetramethyl-1,3,2-dioxaborolan-2-yl)-2-(trifluoromethyl)pyrimidine and 4,4-difluorocyclohexanecarboxylic acid using a procedure analogous to that found in Intermediate B-29, step 1. ESI-MS m/z calc. 310.09, found 311.1 (M+1)⁺.

### Intermediate B-31

### 2-[4-(3,3-difluorocyclobutyl)-2-[4-(trifluoromethyl)cyclohexyl]phenyl]-4,4,5,5-tetramethyl-1,3,2-dioxaborolane (trans isomer)

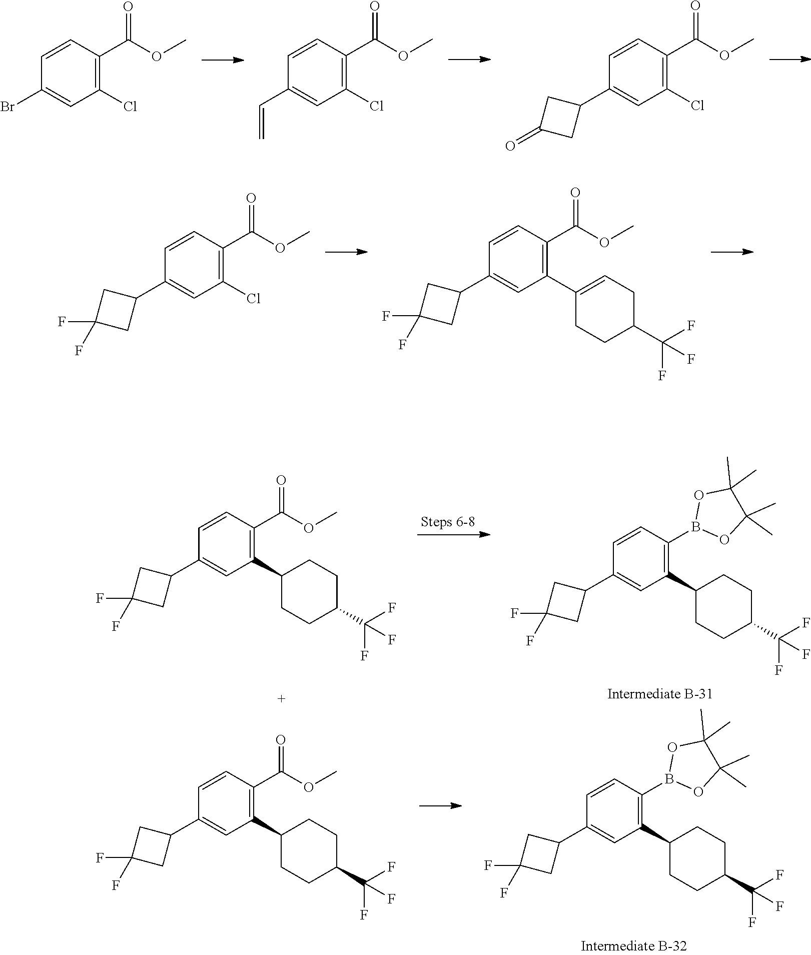

### Step 1: methyl 2-chloro-4-vinyl-benzoate

A mixture of methyl 4-bromo-2-chlorobenzoate (2.0 g, 8.0 mmol), potassium vinyltrifluoroborate (1.3 g, 9.7 mmol), cesium carbonate (5.25 g, 16.1 mmol) in 1,4-dioxane (40 mL) and water (6 mL) was degassed by nitrogen bubbling for 15 min and then Pd(dppf)Cl₂.DCM (197 mg, 0.241 mmol) was added. The mixture was heated at 100° C. and stirred at this temperature for 19 h. Once cooled to room temperature, the reaction mixture was filtered over Celite® and rinsed with ethyl acetate (50 mL). The filtrate was diluted with water (100 mL) and the layers were separated. The aqueous layer was extracted with ethyl acetate (2×50 mL), and the combined organic layers washed with brine (50 mL), dried over sodium sulfate, filtered and concentrated under reduced pressure. The resulting residue was adsorbed on silica gel under vacuum and purified by silica gel chromatography (0-10% ethyl acetate/heptanes) to provide methyl 2-chloro-4-vinyl-benzoate (1.14 g, 71%) as a colorless oil. ESI-MS m/z calc. 196.03, found 197.1 (M+1)⁺. ¹H NMR (400 MHz, CDCl₃) δ 7.83 (d, J=8.1 Hz, 1H), 7.48 (d, J=1.2 Hz, 1H), 7.33 (dd, J=8.1, 1.2 Hz, 1H), 6.68 (dd, J=17.6, 10.9 Hz, 1H), 5.87 (d, J=17.6 Hz, 1H), 5.43 (d, J=10.9 Hz, 1H), 3.94 (s, 3H).

### Step 2: methyl 2-chloro-4-(3-oxocyclobutyl)benzoate

To a solution of N,N-dimethylacetamide (1.3 g, 1.4 mL, 15 mmol) in 1,2-dichloroethane (4 mL) under nitrogen at −15° C. was added a solution of trifluoromethanesulfonic anhydride (4.2 g, 2.5 mL, 15 mmol) in 1,2-dichloroethane (9 mL) dropwise. The mixture was then stirred at −15° C. for 10 min. A separate vial was then charged with methyl 2-chloro-4-vinyl-benzoate (940 mg, 4.78 mmol) and 1,2-dichloroethane (9 mL) followed by a solution of 2,4,6-trimethylpyridine (920 mg, 1.0 mL, 7.6 mmol) in 1,2-dichloroethane (3 mL) was added dropwise under stirring at −15° C. The resulting mixture was added dropwise to the first solution and then the combined mixture heated at 80° C. for 65 h. After cooling to room temperature, the mixture was carefully quenched by water addition (50 mL) and extracted with ethyl acetate (2×100 mL). The combined organic layers were washed with brine (2×50 mL), dried over sodium sulfate, filtered and concentrated under reduced pressure provided 1.82 g of a brown oil. The crude product was adsorbed on silica gel under vacuum and purified by silica gel chromatography (0-30% ethyl acetate/heptanes) to provide methyl 2-chloro-4-(3-oxocyclobutyl)benzoate (271 mg, 22%) as a yellow oil (93% purity). ESI-MS m/z calc. 238.04, found 239.0 (M+1)⁺. ¹H NMR (400 MHz, CDCl₃) δ 7.86 (d, J=8.2 Hz, 1H), 7.40 (d, J=0.9 Hz, 1H), 7.25 (br d, J=1.1 Hz, 1H), 3.94 (s, 3H), 3.74-3.66 (m, 1H), 3.61-3.49 (m, 2H), 3.32-3.22 (m, 2H).

### Step 3: methyl 2-chloro-4-(3,3-difluorocyclobutyl)benzoate

To a solution of methyl 2-chloro-4-(3-oxocyclobutyl)benzoate (270 mg, 1.13 mmol) in dichloromethane (1 mL) was added Deoxo-Fluor® (2 mL of 50% w/v in THF, 4.5 mmol). The reaction mixture was stirred at room temperature for 40 h, then poured into stirring mixture of saturated aqueous sodium bicarbonate (25 mL) and dichloromethane (20 mL). The aqueous layer was extracted with additional dichloromethane (2×30 mL). The organic extracts were combined, dried over sodium sulfate, filtered and evaporated under reduced pressure. The crude product was adsorbed on silica gel under vacuum and purified by silica gel chromatography (0-10% ethyl acetate/heptane) to provide methyl 2-chloro-4-(3,3-difluorocyclobutyl)benzoate (235 mg, 78%) as a colorless oil. ESI-MS m/z calc. 260.04, found 261.0 (M+1)⁺. ¹H NMR (400 MHz, CDCl₃) δ 7.84 (d, J=8.1 Hz, 1H), 7.33 (d, J=0.9 Hz, 1H), 7.19 (dd, J=8.1, 1.1 Hz, 1H), 3.94 (s, 3H), 3.41 (quin, J=8.3 Hz, 1H), 3.12-2.97 (m, 2H), 2.77-2.61 (m, 2H). 19F NMR (377 MHz, CDCl₃) δ −81.94-−82.83 (m, 1F), −98.38-−99.29 (m, 1F).

### Step 4: methyl 4-(3,3-difluorocyclobutyl)-2-[4-(trifluoromethyl)cyclohexen-1-yl]benzoate

Methyl 4-(3,3-difluorocyclobutyl)-2-[4-(trifluoromethyl)cyclohexen-1-yl]benzoate was prepared from methyl 2-chloro-4-(3,3-difluorocyclobutyl)benzoate and 4,4,5,5-tetramethyl-2-[4-(trifluoromethyl)cyclohexen-1-yl]-1,3,2-dioxaborolane using a procedure analogous to that found in Intermediate B-24, step 1 using potassium phosphate as base and XPhos Pd G2 catalyst. ESI-MS m/z calc. 374.13, found 375.1 (M+1)⁺.

### Step 5: methyl 4-(3,3-difluorocyclobutyl)-2-[4-(trifluoromethyl)cyclohexyl]benzoate (trans isomer) and methyl 4-(3,3-difluorocyclobutyl)-2-[4-(trifluoromethyl)cyclohexyl]benzoate (cis isomer)

A solution of methyl 4-(3,3-difluorocyclobutyl)-2-[4-(trifluoromethyl)cyclohexen-1-yl]benzoate (1.05 g, 2.53 mmol) in ethyl acetate (42 mL) was stirred with 10% Pd/C (wet, 540 mg, 0.254 mmol) under hydrogen atmosphere at room temperature for 19 h. The reaction mixture was filtered on Celite® and the pad was rinsed with ethyl acetate (100 mL). The filtrate was evaporated to provide the product as a 78/22 ratio of diastereomers. Purification by silica gel chromatography (0-8% ethyl acetate/heptanes) provided methyl 4-(3,3-difluorocyclobutyl)-2-[4-(trifluoromethyl)cyclohexyl]benzoate (minor trans isomer) (225 mg, 20%). ¹H NMR (400 MHz, CDCl₃) δ 7.83-7.78 (m, 1H), 7.19 (s, 1H), 7.14 (d, J=8.2 Hz, 1H), 3.91 (s, 3H), 3.50-3.35 (m, 2H), 3.03 (tdd, J=14.0, 9.0, 5.1 Hz, 2H), 2.76-2.60 (m, 2H), 2.18-2.11 (m, 1H), 2.11-1.99 (m, 4H), 1.62-1.43 (m, 4H). ¹⁹F NMR (377 MHz, CDCl₃) δ −73.73 (d, J=8.2 Hz, 3F), −81.72-−82.65 (m, 1F), −98.51-−99.35 (m, 1F). ESI-MS m/z calc. 376.15, found 377.2 (M+1)⁺; Retention time: 2.43 min. and methyl 4-(3,3-difluorocyclobutyl)-2-[4-(trifluoromethyl)cyclohexyl]benzoate (major cis isomer) (744 mg, 72%). ¹H NMR (400 MHz, CDCl₃) δ 7.79 (d, J=8.1 Hz, 1H), 7.20 (s, 1H), 7.14 (dd, J=8.1, 1.2 Hz, 1H), 3.90 (s, 3H), 3.56-3.46 (m, 1H), 3.45-3.36 (m, 1H), 3.04 (tdd, J=14.0, 9.0, 5.1 Hz, 2H), 2.76-2.60 (m, 2H), 2.48-2.33 (m, 1H), 2.20-2.09 (m, 2H), 1.84-1.70 (m, 6H).

¹⁹F NMR (377 MHz, CDCl₃) δ −66.68 (br d, J=12.3 Hz, 3F), −81.94-−82.62 (m, 1F), −98.59-−99.32 (m, 1F). ESI-MS m/z calc. 376.15, found 377.2 (M+1)⁺; Retention time: 2.41 min. Reverse phase retention times were determined using a Kinetex Polar C18 (50×3.0 mm, 2.6 m particle), 5-95% acetonitrile/0.1% aqueous formic acid) over 3 min. Flow rate=1.2 mL/min.

### Step 6: 4-(3,3-difluorocyclobutyl)-2-[4-(trifluoromethyl)cyclohexyl]benzoic acid (trans isomer)

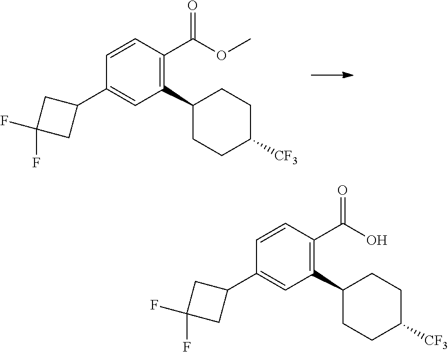 [SMILES: COC(=O)c1ccc(C2CC(F)(F)C2)cc1[C@H]1CC[C@H](C(F)(F)F)CC1 ; O=C(O)c1ccc(C2CC(F)(F)C2)cc1[C@H]1CC[C@H](C(F)(F)F)CC1]

4-(3,3-difluorocyclobutyl)-2-[4-(trifluoromethyl)cyclohexyl]benzoic acid (trans isomer) was prepared from methyl 4-(3,3-difluorocyclobutyl)-2-[4-(trifluoromethyl)cyclohexyl]benzoate (trans isomer) using a procedure analogous to that found in Intermediate B-6, step 4. ¹H NMR (400 MHz, CDCl₃) δ 10.93 (br s, 1H), 7.99 (d, J=8.1 Hz, 1H), 7.23 (s, 1H), 7.19 (d, J=8.2 Hz, 1H), 3.65-3.55 (m, 1H), 3.49-3.38 (m, 1H), 3.05 (tdd, J=14.0, 9.0, 5.1 Hz, 2H), 2.79-2.63 (m, 2H), 2.22-2.00 (m, 5H), 1.63-1.44 (m, 4H). ¹⁹F NMR (377 MHz, CDCl₃) δ −73.72 (d, J=8.2 Hz, 3F), −81.89-−82.59 (m, 1F), −98.49-−99.28 (m, 1F).

### Step 7: 1-bromo-4-(3,3-difluorocyclobutyl)-2-[4-(trifluoromethyl)cyclohexyl]benzene (trans isomer)

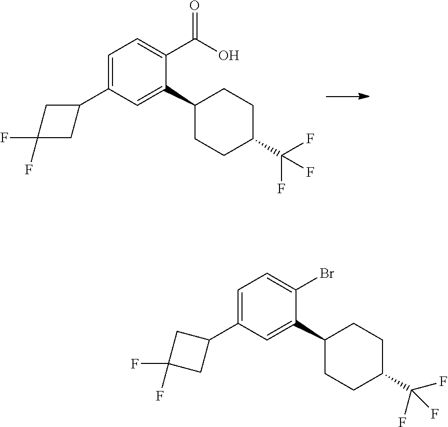 [SMILES: O=C(O)c1ccc(C2CC(F)(F)C2)cc1[C@H]1CC[C@H](C(F)(F)F)CC1 ; FC1(F)CC(c2ccc(Br)c([C@H]3CC[C@H](C(F)(F)F)CC3)c2)C1]

1-Bromo-4-(3,3-difluorocyclobutyl)-2-[4-(trifluoromethyl)cyclohexyl]benzene (trans isomer) was prepared from 4-(3,3-difluorocyclobutyl)-2-[4-(trifluoromethyl)cyclohexyl]benzoic acid (trans isomer) using a procedure analogous to that found in Intermediate B-12, step 3. ¹H NMR (400 MHz, CDCl₃) δ 7.51 (d, J=8.2 Hz, 1H), 7.05 (d, J=2.3 Hz, 1H), 7.01-6.89 (m, 1H), 3.42-3.23 (m, 1H), 3.10-2.91 (m, 3H), 2.76-2.49 (m, 2H), 2.17-2.04 (m, 4H), 1.61-1.36 (m, 5H). ¹⁹F NMR (376 MHz, CDCl₃) δ −73.74, −82.16 (d, J=194.1 Hz), −99.12 (d, J=194.2 Hz).

### Step 8: 2-[4-(3,3-difluorocyclobutyl)-2-[4-(trifluoromethyl)cyclohexyl]phenyl]-4,4,5,5-tetramethyl-1,3,2-dioxaborolane (trans isomer)

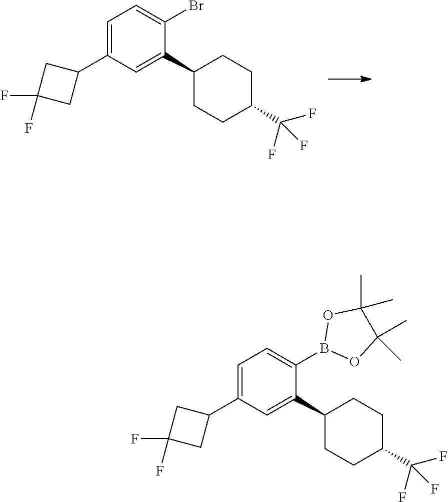 [SMILES: FC1(F)CC(c2ccc(Br)c([C@H]3CC[C@H](C(F)(F)F)CC3)c2)C1 ; CC1(C)OB(c2ccc(C3CC(F)(F)C3)cc2[C@H]2CC[C@H](C(F)(F)F)CC2)OC1(C)C]

2-[4-(3,3-Difluorocyclobutyl)-2-[4-(trifluoromethyl)cyclohexyl]phenyl]-4,4,5,5-tetramethyl-1,3,2-dioxaborolane (trans isomer) was prepared from 1-bromo-4-(3,3-difluorocyclobutyl)-2-[4-(trifluoromethyl)cyclohexyl]benzene (trans isomer) using a procedure analogous to that found in Intermediate B-22, step 4 using Pd(dppf)Cl₂ catalyst and 1,4-dioxane as solvent. ¹H NMR (400 MHz, CDCl₃) δ 7.75 (d, J=8.2 Hz, 1H), 7.11-7.04 (m, 2H), 3.43-3.26 (m, 2H), 3.06-2.91 (m, 2H), 2.76-2.57 (m, 2H), 2.16-2.03 (m, 3H), 1.97 (s, 1H), 1.53-1.39 (m, 5H), 1.34 (s, 12H). ¹⁹F NMR (376 MHz, CDCl₃) δ −73.70, −81.97 (d, J=193.5 Hz), −99.39 (d, J=192.8 Hz).

### Intermediate B-32

### 2-[4-(3,3-difluorocyclobutyl)-2-[4-(trifluoromethyl)cyclohexyl]phenyl]-4,4,5,5-tetramethyl-1,3,2-dioxaborolane (cis isomer)

### Step 1: 4-(3,3-difluorocyclobutyl)-2-[4-(trifluoromethyl)cyclohexyl]benzoic acid (cis isomer)

4-(3,3-Difluorocyclobutyl)-2-[4-(trifluoromethyl)cyclohexyl]benzoic acid (cis isomer) was prepared from methyl 4-(3,3-difluorocyclobutyl)-2-[4-(trifluoromethyl)cyclohexyl]benzoate (cis isomer, from Intermediate B-31, step 5) using a procedure analogous to that found in Intermediate B-6, step 4. ¹H NMR (400 MHz, CDCl₃) δ 11.53-10.26 (m, 1H), 7.97 (d, J=8.1 Hz, 1H), 7.25 (s, 1H), 7.19 (d, J=8.2 Hz, 1H), 3.72-3.60 (m, 1H), 3.49-3.39 (m, 1H), 3.06 (tdd, J=14.0, 9.0, 5.3 Hz, 2H), 2.79-2.62 (m, 2H), 2.48-2.34 (m, 1H), 2.22-2.09 (m, 2H), 1.87-1.72 (m, 6H). ¹⁹F NMR (377 MHz, CDCl₃) δ −66.67 (br d, J=12.3 Hz, 3F), −81.97-−82.67 (m, 1F), −98.45-−99.21 (m, 1F). ESI-MS m/z calc. 362.13, found 363.0 (M+1)⁺; Retention time: 3.42 min.

### Step 2: 1-bromo-4-(3,3-difluorocyclobutyl)-2-[4-(trifluoromethyl)cyclohexyl]benzene (cis isomer)

1-Bromo-4-(3,3-difluorocyclobutyl)-2-[4-(trifluoromethyl)cyclohexyl]benzene (cis isomer) was prepared from 4-(3,3-difluorocyclobutyl)-2-[4-(trifluoromethyl)cyclohexyl]benzoic acid (cis isomer) using a procedure analogous to that found in Intermediate B-12, step 3.

### Step 3: 2-[4-(3,3-difluorocyclobutyl)-2-[4-(trifluoromethyl)cyclohexyl]phenyl]-4,4,5,5-tetramethyl-1,3,2-dioxaborolane (cis isomer)

2-[4-(3,3-Difluorocyclobutyl)-2-[4-(trifluoromethyl)cyclohexyl]phenyl]-4,4,5,5-tetramethyl-1,3,2-dioxaborolane (cis isomer) was prepared from 1-bromo-4-(3,3-difluorocyclobutyl)-2-[4-(trifluoromethyl)cyclohexyl]benzene (cis isomer) using a procedure analogous to that found in Intermediate B-22, step 4.

### Intermediate B-33

### [2-(2-ethyl-4-fluoro-phenoxy)-5-(trifluoromethyl)-3-pyridyl]boronic acid

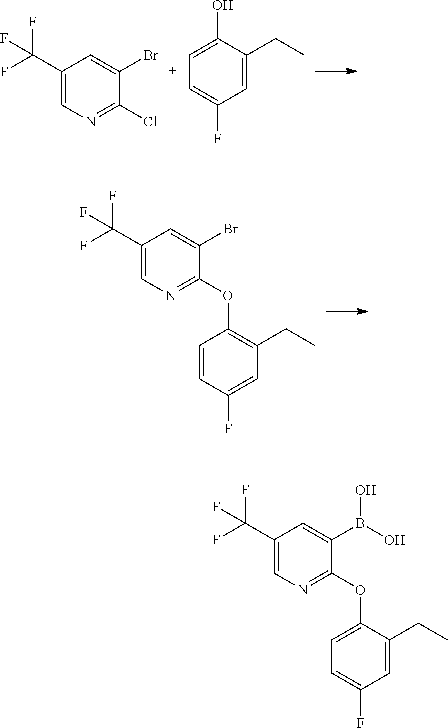

### Step 1: 3-bromo-2-(2-ethyl-4-fluoro-phenoxy)-5-(trifluoromethyl)pyridine

A mixture of 3-bromo-2-chloro-5-(trifluoromethyl)pyridine (750 mg, 2.88 mmol) and 2-ethyl-4-fluoro-phenol (502 mg, 3.58 mmol) in DMSO (6.0 mL) was treated with cesium carbonate (2.35 g, 7.21 mmol) and stirred at 100° C. for 20 min. The mixture was allowed to cool to room temperature and partitioned between diethyl ether and water. The organic layer was separated and washed with 1 N NaOH (2×) and brine, dried over magnesium sulfate, filtered and concentrated. Purification by silica gel chromatography (80 g silica, 0-30% ethyl acetate/hexane) provided 3-bromo-2-(2-ethyl-4-fluoro-phenoxy)-5-(trifluoromethyl)pyridine (1.040 g, 99%). ESI-MS m/z calc. 362.99, found 364.1 (M+1)⁺. ¹H NMR (400 MHz, CDCl₃) δ 8.28 (dd, J=2.2, 1.1 Hz, 1H), 8.15 (d, J=2.2 Hz, 1H), 7.08-7.00 (m, 2H), 6.96 (ddd, J=8.8, 7.8, 3.1 Hz, 1H), 2.50 (q, J=7.5 Hz, 2H), 1.18 (t, J=7.5 Hz, 3H). ¹⁹F NMR (376 MHz, CDCl₃) δ −61.57, −115.62-−117.22 (m).

### Step 2: [2-(2-ethyl-4-fluoro-phenoxy)-5-(trifluoromethyl)-3-pyridyl]boronic acid

To a stirring solution of 3-bromo-2-(2-ethyl-4-fluoro-phenoxy)-5-(trifluoromethyl)pyridine (210 mg, 0.577 mmol) in diethyl ether (3.5 mL) at −78° C. was added n-BuLi in hexanes (250 μL of 2.5 M, 0.63 mmol) dropwise under nitrogen atmosphere. The mixture was stirred at this temperature for 20 min then trimethyl borate (100 μL, 0.881 mmol) added dropwise. The mixture was allowed to warm to room temperature and stirred for 1 h. The mixture was diluted with saturated aqueous ammonium chloride and extracted with ethyl acetate. The organic extract was dried over sodium sulfate, filtered and concentrated to provide [2-(2-ethyl-4-fluoro-phenoxy)-5-(trifluoromethyl)-3-pyridyl]boronic acid (185 mg, 97%). ESI-MS m/z calc. 329.09, found 330.2 (M+1)⁺.

### Intermediate B-34

### 3-tert-butyl-1-(4,4-difluorocyclohexyl)-4-methyl-5-(4,4,5,5-tetramethyl-1,3,2-dioxaborolan-2-yl)pyrazole

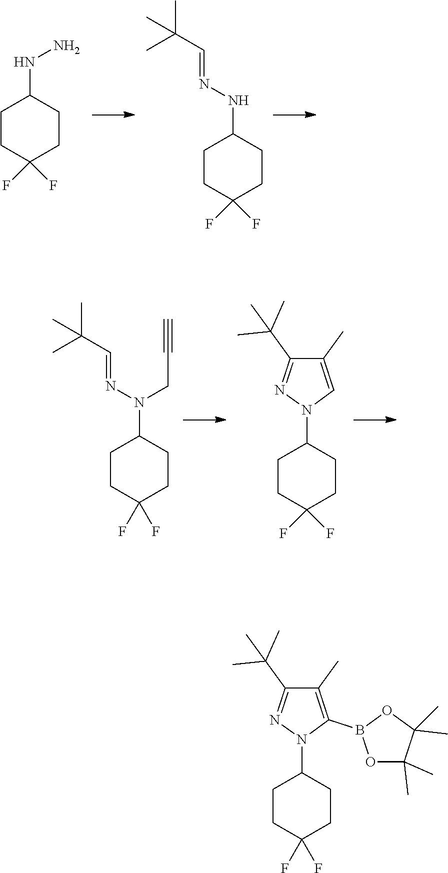 [SMILES: NNC1CCC(F)(F)CC1 ; CC(C)(C)/C=N/NC1CCC(F)(F)CC1 ; C#CCN(/N=C/C(C)(C)C)C1CCC(F)(F)CC1 ; Cc1cn(C2CCC(F)(F)CC2)nc1C(C)(C)C ; Cc1c(C(C)(C)C)nn(C2CCC(F)(F)CC2)c1B1OC(C)(C)C(C)(C)O1]

### Step 1: N-[(E)-2,2-dimethylpropylideneamino]-4,4-difluoro-cyclohexanamine

To a solution of (4,4-difluorocyclohexyl)hydrazine (2 HCl) (29.15 g, 113.7 mmol) in methanol (500 mL) was added 2,2-dimethylpropanal (9.80 g, 12.4 mL, 114 mmol) and the mixture stirred at room temperature overnight. The solvent was removed under reduced pressure and the residue triturated using heptane to provide N-[(E)-2,2-dimethylpropylideneamino]-4,4-difluoro-cyclohexanamine hydrochloride (29.1 g, 98%). ¹H NMR (400 MHz, CDCl₃) δ 11.85 (br s, 2H), 8.40 (s, 1H), 3.49-3.38 (m, 1H), 2.33-2.23 (m, 4H), 2.06-1.94 (m, 2H), 1.91-1.72 (m, 2H), 1.16 (s, 9H)

### Step 2: N-[(E)-2,2-dimethylpropylideneamino]-4,4-difluoro-N-prop-2-ynyl-cyclohexanamine

To a solution of N-[(E)-2,2-dimethylpropylideneamino]-4,4-difluoro-cyclohexanamine hydrochloride (10.0 g, 37.3 mmol) in DMF (160 mL) was added cesium carbonate (21.2 g, 65.1 mmol) followed by a solution of propargyl bromide in toluene (7.7 mL of 80% w/v, 52 mmol). The mixture was stirred at room temperature for 16 h. The mixture was filtered, washed with diethyl ether (200 mL) and the filtrate diluted with water (200 mL). The organic layer was collected and the aqueous was extracted with additional diethyl ether (100 mL). The combined organic extracts were washed with water and brine, dried over sodium sulfate and concentrated. Purification by silica gel chromatography (2-10% EtOAc/heptanes) provided N-[(E)-2,2-dimethylpropylideneamino]-4,4-difluoro-N-prop-2-ynyl-cyclohexanamine (6.1 g, 60%). ¹H NMR (400 MHz, CDCl₃) δ 6.85 (s, 1H), 3.80 (d, J=2.3 Hz, 2H), 3.16 (q, J=4.4 Hz, 1H), 2.19 (t, J=2.5 Hz, 1H), 2.17-2.09 (m, 2H), 1.95-1.72 (m, 6H), 1.07-1.05 (m, 9H). ESI-MS m/z calc. 256.18, found 257.15 (M+1)⁺.

### Step 3: 3-tert-butyl-1-(4,4-difluorocyclohexyl)-4-methyl-pyrazole

To a solution of N-[(E)-2,2-dimethylpropylideneamino]-4,4-difluoro-N-prop-2-ynyl-cyclohexanamine (1.53 g, 5.51 mmol) in acetonitrile (30 mL) was addedepotassium t-butoxide (670 mg, 5.97 mmol) in portions. The mixture was stirred at room temperature for 30 min. The reaction was diluted with water (50 mL) and extracted with ethyl acetate (2×50 mL). The combined extracts were washed with brine, dried over sodium sulfate, filtered and concentrated. Purification by silica gel chromatography (2-10% ethyl acetate/heptanes) provided 3-tert-butyl-1-(4,4-difluorocyclohexyl)-4-methyl-pyrazole (1.38 g, 92%). ¹H NMR (400 MHz, CDCl₃) δ 7.08 (s, 1H), 4.19-4.09 (m, 1H), 2.26-2.14 (m, 4H), 2.12 (d, J=0.9 Hz, 3H), 2.04-1.80 (m, 4H), 1.32 (s, 9H). ESI-MS m/z calc. 256.18, found 257.15 (M+1)⁺.

### Step 4: 5-bromo-3-tert-butyl-1-(4,4-difluorocyclohexyl)-4-methyl-pyrazole

To 3-tert-butyl-1-(4,4-difluorocyclohexyl)-4-methyl-pyrazole (584 mg, 2.16 mmol) in acetonitrile (20 mL) was added NBS (501 mg, 2.82 mmol). The mixture was stirred at room temperature for 1 h then concentrated in vacuo (bath temperature maintained at 35° C.). Purification by silica gel chromatography (2-10% ethyl acetate/heptanes) provided 5-bromo-3-tert-butyl-1-(4,4-difluorocyclohexyl)-4-methyl-pyrazole (690 mg, 91%). ¹H NMR (400 MHz, CDCl₃) δ 4.36-4.27 (m, 1H), 2.38-2.17 (m, 4H), 2.09 (s, 3H), 2.00-1.82 (m, 4H), 1.29 (s, 9H). ESI-MS m/z calc. 334.09, found 335.1 (M+1)⁺.

### Step 5: 3-tert-butyl-1-(4,4-difluorocyclohexyl)-4-methyl-5-(4,4,5,5-tetramethyl-1,3,2-dioxaborolan-2-yl)pyrazole

To a solution of 5-bromo-3-tert-butyl-1-(4,4-difluorocyclohexyl)-4-methyl-pyrazole (7.37 g, 20.9 mmol) in THF (110 mL) at −78° C. under argon was added n-BuLi in hexanes (21 mL of 1.6 M, 34 mmol) dropwise. The mixture was stirred at this temperature for 30 minutes when a solution of 2-isopropoxy-4,4,5,5-tetramethyl-1,3,2-dioxaborolane (7.78 g, 41.8 mmol) in THF (15 mL) was added. The mixture was stirred at −78° C. for 1 h then allowed to warm to room temperature over 4 h. Saturated aqueous ammonium chloride (100 mL) was added and the mixture extracted with ethyl acetate (2×75 mL). The combined extracts were washed with water and brine, dried over sodium sulfate and concentrated. Purification by silica gel chromatography (2-10% EtOAc/heptanes) provided 3-tert-butyl-1-(4,4-difluorocyclohexyl)-4-methyl-5-(4,4,5,5-tetramethyl-1,3,2-dioxaborolan-2-yl)pyrazole (4.38 g, 54%). ¹H NMR (400 MHz, CDCl₃) δ 4.80-4.71 (m, 1H), 2.35 (s, 3H), 2.35-2.19 (m, 4H), 2.05-1.82 (m, 4H), 1.35 (m, 21H). ESI-MS m/z calc. 382.26, found 383.29 (M+1)⁺.

### Intermediate B-35

### 5-(3,4-difluoro-2-methoxy-phenoxy)-3-methyl-4-(4,4,5,5-tetramethyl-1,3,2-dioxaborolan-2-yl)-2-(trifluoromethyl)pyridine

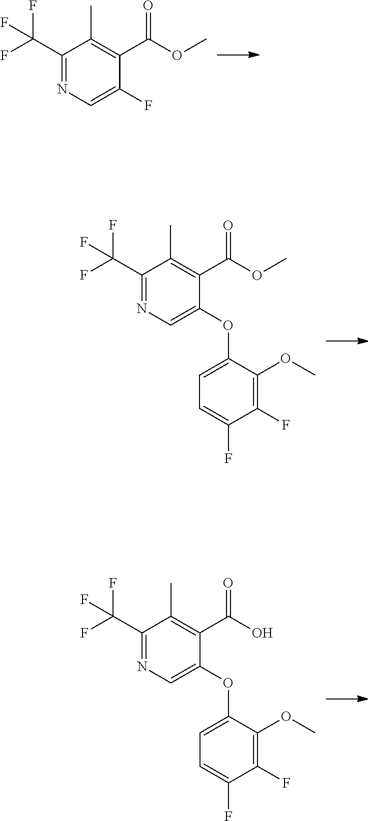 [SMILES: COC(=O)c1c(F)cnc(C(F)(F)F)c1C ; COC(=O)c1c(Oc2ccc(F)c(F)c2OC)cnc(C(F)(F)F)c1C ; COc1c(Oc2cnc(C(F)(F)F)c(C)c2C(=O)O)ccc(F)c1F]

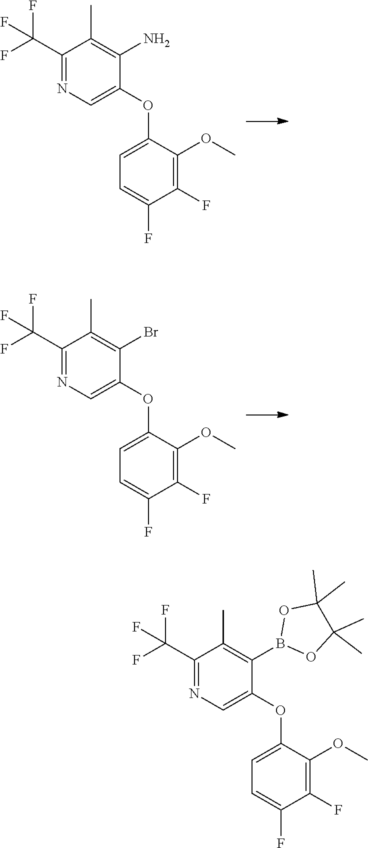 [SMILES: COc1c(Oc2cnc(C(F)(F)F)c(C)c2N)ccc(F)c1F ; COc1c(Oc2cnc(C(F)(F)F)c(C)c2Br)ccc(F)c1F ; COc1c(Oc2cnc(C(F)(F)F)c(C)c2B2OC(C)(C)C(C)(C)O2)ccc(F)c1F]

### Step 1: methyl 5-(3,4-difluoro-2-methoxy-phenoxy)-3-methyl-2-(trifluoromethyl)pyridine-4-carboxylate

Methyl 5-(3,4-difluoro-2-methoxy-phenoxy)-3-methyl-2-(trifluoromethyl)pyridine-4-carboxylate was prepared from methyl 5-fluoro-3-methyl-2-(trifluoromethyl)pyridine-4-carboxylate and 3,4-difluoro-2-methoxy-phenol using a procedure analogous to that found in Intermediate B-1, step 1 and using toluene as the solvent. ESI-MS m/z calc. 377.07, found 378.04 (M+1)⁺. ¹H NMR (400 MHz, CDCl₃) δ 7.91 (s, 1H), 6.95-6.85 (m, 2H), 3.98 (s, 3H), 3.91 (d, J=1.8 Hz, 3H), 2.46 (d, J=1.4 Hz, 3H)

### Step 2: 5-(3,4-difluoro-2-methoxy-phenoxy)-3-methyl-2-(trifluoromethyl)pyridine-4-carboxylic acid

5-(3,4-Difluoro-2-methoxy-phenoxy)-3-methyl-2-(trifluoromethyl)pyridine-4-carboxylic acid was prepared from methyl 5-(3,4-difluoro-2-methoxy-phenoxy)-3-methyl-2-(trifluoromethyl)pyridine-4-carboxylate using a procedure analogous to that found in Intermediate B-6, step 4. ESI-MS m/z calc. 363.05, found 364.01 (M+1)⁺˜. ¹H NMR (400 MHz, DMSO-d₆) δ 8.11 (s, 1H), 7.29-7.22 (in, 1H), 7.12-7.08 (in, 1H), 3.86 (d, J=0.9 Hz, 3H), 2.43 (d, J=1.8 Hz, 3H). COOH peak not observed. ¹⁹F NMR (376 MHz, DMSO-d₆) δ−62.6 (s, 3F), −138.9 (dq, J=21.3, 5.0 Hz, 1F), −152.0 (dd, J=21.5, 8.2 Hz, 1F).

### Step 3: 5-(3,4-difluoro-2-methoxy-phenoxy)-3-methyl-2-(trifluoromethyl)pyridin-4-amine

5-(3,4-Difluoro-2-methoxy-phenoxy)-3-methyl-2-(trifluoromethyl)pyridin-4-amine was prepared from 5-(3,4-difluoro-2-methoxy-phenoxy)-3-methyl-2-(trifluoromethyl)pyridine-4-carboxylic acid using a procedure analogous to that found in Intermediate B-6, steps 5 and 6. ESI-MS m/z calc. 334.07, found 334.97 (M+1)⁺. ¹H NMR (400 MHz, CDCl₃) δ 7.75 (s, 1H), 6.86 (td, J=9.4, 8.1 Hz, 1H), 6.76 (qd, J=4.8, 2.2 Hz, 1H), 4.63 (s, 2H), 3.94 (d, J=1.8 Hz, 3H), 2.28 (d, J=0.7 Hz, 3H). ¹⁹F NMR (376 MHz, CDCl₃) δ −63.4 (d, J=19.1 Hz, 3F), −138.8 (dq, J=20.0, 4.7 Hz, 1F), −151.1 (dd, J=19.9, 7.6 Hz, 1F)

### Step 4: 4-bromo-5-(3,4-difluoro-2-methoxy-phenoxy)-3-methyl-2-(trifluoromethyl)pyridine

4-Bromo-5-(3,4-difluoro-2-methoxy-phenoxy)-3-methyl-2-(trifluoromethyl)pyridine was prepared from 5-(3,4-difluoro-2-methoxy-phenoxy)-3-methyl-2-(trifluoromethyl)pyridin-4-amine using a procedure analogous to that found in Intermediate B-3, step 6. ESI-MS n z calc. 396.97, found 398.0 (M+1)⁺. ¹H NMR (400 MHz, CDCl₃) δ 7.79 (s, 1H), 6.96-6.82 (m, 2H), 3.94 (d, J=1.8 Hz, 3H), 2.63 (d, J=1.3 Hz, 3H). ¹⁹F NMR (376 MHz, CDCl₃) δ −63.9 (s, 3F), −137.4 (qd, J=9.8, 4.9 Hz, 1F), −150.7 (dd, J=20.1, 7.8 Hz, 1F)

### Step 5: 5-(3,4-difluoro-2-methoxy-phenoxy)-3-methyl-4-(4,4,5,5-tetramethyl-1,3,2-dioxaborolan-2-yl)-2-(trifluoromethyl)pyridine

5-(3,4-Difluoro-2-methoxy-phenoxy)-3-methyl-4-(4,4,5,5-tetramethyl-1,3,2-dioxaborolan-2-yl)-2-(trifluoromethyl)pyridine was prepared from 4-bromo-5-(3,4-difluoro-2-methoxy-phenoxy)-3-methyl-2-(trifluoromethyl)pyridine using a procedure analogous to that found in Intermediate B-9, step 7. ESI-MS m/z calc. 445.15, found 446.18 (M+1)⁺. ¹H NMR (400 MHz, CDCl₃) δ 7.83 (s, 1H), 6.87 (td, J=9.3, 7.9 Hz, 1H), 6.79 (qd, J=4.7, 2.2 Hz, 1H), 3.92 (d, J=1.8 Hz, 3H), 2.53 (t, J=1.6 Hz, 3H), 1.37 (s, 12H). ¹⁹F NMR (376 MHz, CDCl₃) δ −64.0 (d, J=1.3 Hz, 3F), −138.7 (dq, J=20.1, 4.7 Hz, 1F), −151.3 (dd, J=20.0, 7.8 Hz, 1F)

### Example 2

### Method for Metal-Mediated Cross-Coupling Between Intermediate-A and Intermediate-B

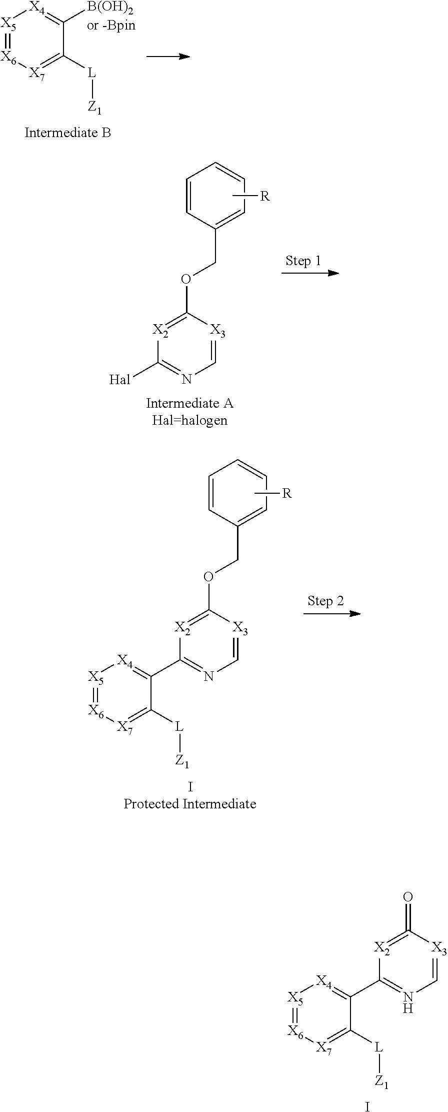

Step 1: A mixture of Intermediate A (1 eq), Intermediate B (1-2 eq, custom or commercial boronic acid or boronic ester), Palladium source (1-5 mol %, e.g. PdCl₂(dppf) or PdCl₂(dtbpf), base (2-3 eq, eg. potassium phosphate) in organic solvent (e.g. dioxane, DMSO, toluene) and water is degassed with nitrogen bubbling and stirred under inert atmosphere at a temperature ranging from room temperature to 120° C. The reaction mixture is filtered and purified via silica gel column chromatography or reverse phase HPLC to obtain protected Intermediate I. In some cases the pyridone protecting group will be cleaved during the cross-coupling conditions leading directly to product I.

Step 2: A mixture of the protected intermediate I and Pd/C is stirred in the appropriate solvent (e.g. methanol, ethanol, or ethyl acetate) under an atmosphere of hydrogen. The reaction mixture is filtered, concentrated, and purified via silica gel column chromatography or reverse phase column chromatography to provide the desired product I.

Alternatively, a solution of protected intermediate I in the appropriate solvent (DCM, 1,4-dioxane or toluene) is treated with acid (e.g. HCl or TFA) and stirred at room temperature or 60-70° C. The mixture is neutralized and purified via silica gel column chromatography or reverse phase column chromatography to provide the desired product I.

The following compounds in Table 4 were synthesized using the corresponding Intermediates A and Intermediates B boronic acid/esters using Suzuki cross-coupling conditions described above.

TABLE 4

| | | MW & | |
|---|---|---|---|
| Cmpd | | Found | |
| No. | Compound Name | [M + H]⁺ | NMR (shifts in ppm) |
| 1 | methyl 6-[5-[2-methoxy-4- | 532.11; | ¹H NMR (400 MHz, CD₃OD) δ 7.99 (s, |
| | (trifluoromethoxy)phenoxy]-3- | 533.12 | 1H), 7.24 (d, J = 8.8 Hz, 1H), 7.10-7.09 |
| | methyl-2-(trifluoromethyl)-4- | | (m, 1H), 6.95 (d, J = 8.7 Hz, 1H), 6.54 |
| | pyridyl]-2-methyl-4-oxo-1H- | | (s, 1H), 3.92 (s, 3H), 3.82 (s, 3H), 2.44 |
| | pyridine-3-carboxylate | | (s, 3H), 2.40 (s, 3H). |
| 2 | ethyl 6-[6-(3,3- | 512.51; | ¹H NMR (400 MHz, DMSO-d₆) δ 11.66 |
| | difluorocyclobutyl)-4-[(1s,4s)-4- | 513.3 | (s, 1H), 8.46 (s, 1H), 7.40 (s, 1H), 4.29- |
| | (trifluoromethyl)cyclohexyl]-3- | | 4.19 (m, 2H), 3.70-3.62 (m, 2H), 3.05- |
| | pyridyl]-2,5-dimethyl-4-oxo-1H- | | 2.90 (m, 3H), 2.90-2.71 (m, 1H), 2.46- |
| | pyridine-3-carboxylate | | 2.40 (m, 1H), 2.22 (s, 3H), 1.97-1.84 |
| | | | (m, 2H), 1.78-1.46 (m, 9H), 1.27 (t, J = |
| | | | 7.1 Hz, 3H). |
| 3 | 3-acetyl-6-[5-(3,4-difluoro-2- | 468.37; | ¹H NMR (400 MHz, CD₃OD) δ 8.01 (s, |
| | methoxy-phenoxy)-3-methyl-2- | 469.15 | 1H), 7.05-6.96 (m, 2H), 6.44 (s, 1H), |
| | (trifluoromethyl)-4-pyridyl]-2- | | 3.84 (d, J = 1.8 Hz, 3H), 2.53-2.50 (m, |
| | methyl-1H-pyridin-4-one | | 3H), 2.37 (d, J = 1.4 Hz, 3H), 2.34 (s, |
| | | | 3H). |
| 4 | 2-[6-[6-(3,3-difluorocyclobutyl)-4- | 497.5; | ¹H NMR (400 MHz, CD₃OD) δ 8.46 (s, |
| | [4-(trifluoromethyl)cyclohexyl]-3- | 498.6 | 1H), 7.33 (s, 1H), 6.33 (s, 1H), 3.59 (qd, |
| | pyridyl]-2-methyl-4-oxo-1H- | | J = 8.7, 3.0 Hz, 1H), 3.53 (s, 2H), 3.01- |
| | pyridin-3-yl]-N-methyl-acetamide | | 2.84 (m, 4H), 2.82-2.75 (m, 1H), 2.73 |
| | | | (s, 3H), 2.43 (s, 3H), 2.40-2.34 (m, 1H), |
| | | | 2.09-2.02 (m, 2H), 1.86-1.62 (m, 6H). |
| 5 | 2-[6-[6-(3,3-difluorocyclobutyl)-4- | 483.47; | ¹H NMR (400 MHz, CD₃OD) 8 8.46 (s, |
| | [4-(trifluoromethyl)cyclohexyl]-3- | 484.6 | 1H), 7.33 (s, 1H), 6.33 (s, 1H), 3.65- |
| | pyridyl]-2-methyl-4-oxo-1H- | | 3.51 (m, 1H) overlaps with 3.56 (s, 2H), |
| | pyridin-3-yl]acetamide | | 3.01-2.85 (m, 4H), 2.83-2.73 (m, 1H), |
| | | | 2.49-2.34 (m, 1H) overlaps with 3.56 (s, |
| | | | 3H), 2.10-1.99 (m, 2H), 1.85-1.62 (m, |
| | | | 6H) |
| 6 | 6-[6-(3,3-difluorocyclobutyl)-4-14- | 503.53; | ¹H NMR (400 MHz, DMSO-d₆) δ 12.78 |
| | (trifluoromethyl)cyclohexyl]-3- | 504.3 | (s, 1H), 9.54 (s, 2H), 8.52 (s, 1H), 7.43 |
| | pyridyl]-2-methyl-3- | | (s, 1H), 6.55 (s, 1H), 3.94 (s, 3H), 3.84- |
| | (methylsulfonimidoyl)-1H- | | 3.51 (m, 1H), 3.07-2.76 (m, 4H), 2.75- |
| | pyridin-4-one (cis isomer, | | 2.59 (m, 3H), 2.02-1.83 (m, 2H), 1.77- |
| | diastereomer 2 at sulfoximine) | | 1.55 (m, 7H). |
| 7 | 6-[6-(3,3-difluorocyclobutyl)-4-[4- | 503.53; | ¹H NMR (400 MHz, DMSO-d₆) δ 12.76 |
| | (trifluoromethyl)cyclohexyl]-3- | 504.3 | (s, 1H), 8.52 (s, 1H), 7.43 (s, 1H), 6.55 |
| | pyridyl]-2-methyl-3- | | (s, 1H), 3.93 (s, 3H), 3.73-3.67 (m, 2H), |
| | (methylsulfonimidoyl)-1H- | | 3.05-2.77 (m, 4H), 2.73-2.58 (m, 4H), |
| | pyridin-4-one (cis isomer, | | 2.00-1.86 (m, 2H), 1.78-1.51 (m, 6H). |
| | diastereomer 1 at sulfoximine) | | |
| 8 | 6-[5-(3,4-difluoro-2-methoxy- | 503.44; | ¹H NMR (400 MHz, CDCl₃) δ 8.00 (s, |
| | phenoxy)-3-methyl-2- | 504.1 | 1H), 6.92-6.85 (m, 2H), 6.83-6.79 (m, |
| | (trifluoromethyl)-4-pyridyl]-2- | | 1H), 3.90 (d, J = 1.8 Hz, 3H), 3.44 (s, |
| | methyl-3-(methylsulfonimidoyl)- | | 3H), 2.90 (s, 3H), 2.33 (d, J = 1.4 Hz, |
| | 1H-pyridin-4-one | | 3H). |
| 9 | 6-[6-(3,3-difluorocyclobutyl)-4-[4- | 503.53; | ¹H NMR (400 MHz, DMSO-d₆) δ 8.51 |
| | (trifluoromethyl)cyclohexyl]-3- | 504.4 | (s, 1H), 7.39 (s, 1H), 6.54 (s, 1H), 3.73 |
| | pyridyl]-2-methyl-3- | | (s, 3H), 3.71-3.57 (m, 2H), 3.03-2.79 |
| | (methylsulfonimidoyl)-1H- | | (m, 4H), 2.76-2.63 (m, 4H), 2.03-1.84 |
| | pyridin-4-one (cis isomer, mixture | | (m, 2H), 1.78-1.53 (m, 6H). |
| | of epimers at sulfoximine) | | |
| 10 | ethyl 6-[6-(3,3- | 498.49; | ¹H NMR (400 MHz, DMSO-d₆) δ 8.52 |
| | difluorocyclobutyl)-4-[(1s,4s)-4- | 499.5 | (s, 1H), 7.37 (s, 1H), 6.43 (s, 1H), 4.28 |
| | (trifluoromethyl)cyclohexyl]-3- | | (q, J = 6.8 Hz, 2H), 3.68-3.56 (m, 2H), |
| | pyridyl]-2-methyl-4-oxo-1H- | | 3.03-2.78 (m, 4H), 2.80-2.60 (m, 1H), |
| | pyridine-3-carboxylate | | 2.33 (s, 3H), 2.00-1.81 (m, 2H), 1.74- |
| | | | 1.52 (m, 6H), 1.29 (t, J = 7.0 Hz, 3H). |
| 11 | ethyl 2-[6-(3,3- | 512.51; | ¹H NMR (400 MHz, DMSO-d₆) δ 8.47- |
| | difluorocyclobutyl)-4-[(1s,4s)-4- | 513.3 | 8.28 (m, 1H), 7.36 (s, 1H), 3.98-3.82 |
| | (trifluoromethyl)cyclohexyl]-3- | | (m, 2H), 3.73-3.62 (m, 1H), 3.09-2.72 |
| | pyridyl]-5,6-dimethyl-4-oxo-1H- | | (m, 5H), 2.31 (s, 3H), 2.00-1.94 (m, |
| | pyridine-3-carboxylate | | 3H), 1.94-1.85 (m, 2H), 1.75-1.50 (m, |
| | | | 6H), 1.09-1.00 (m, 1H), 0.81-0.70 (m, |
| | | | 3H). |
| 12 | ethyl 6-[5-(3,4-difluoro-2- | 498.4; | ¹H NMR (400 MHz, CD₃OD) δ 8.03 (s, |
| | methoxy-phenoxy)-3-methyl-2- | 499.04 | 1H), 7.09-6.97 (m, 2H), 6.49 (s, 1H), |
| | (trifluoromethyl)-4-pyridyl]-2- | | 4.38 (q, J = 7.0 Hz, 2H), 3.88-3.86 (m, |
| | methyl-4-oxo-1H-pyridine-3- | | 3H), 2.41 (d, J = 17.9 Hz, 6H), 1.37 |
| | carboxylate | | (t, J = 7.3 Hz, 3H). |
| 13 | 6-[2-(3,4-difluoro-2-methyl- | 487.44; | ¹H NMR (400 MHz, CD₃OD) δ 8.44 (s, |
| | phenoxy)-4-methyl-5- | 488.6 | 1H), 7.14 (q, J = 9.2 Hz, 1H), 6.90 (ddd, |
| | (trifluoromethyl)-3-pyridyl]-2- | | J = 9.1, 4.2, 2.1 Hz, 1H), 6.62 (s, 1H), |
| | methyl-3-(methylsulfonimidoyl)- | | 3.41 (s, 3H), 2.79 (s, 3H), 2.44 (d, J = 1.4 |
| | 1H-pyridin-4-one | | Hz, 3H), 2.05 (d, J = 2.1 Hz, 3H). |
| 14 | 6-[2-(3,4-difluoro-2-methyl- | 487.44; | ¹H NMR (400 MHz, CD₃OD) δ 8.44 (s, |
| | phenoxy)-4-methyl-5- | 488.6 | 1H), 7.14 (q, J = 9.3 Hz, 1H), 6.90 (ddd, |
| | (trifluoromethyl)-3-pyridyl]-2- | | J = 9.1, 4.1, 2.1 Hz, 1H), 6.62 (s, 1H), |
| | methyl-3-(methylsulfonimidoyl)- | | 3.41 (s, 3H), 2.79 (s, 3H), 2.44 (d, J = 1.5 |
| | 1H-pyridin-4-one | | Hz, 3H), 2.05 (d, J = 2.2 Hz, 3H). |
| 15 | 6-[2-(3,4-difluoro-2-methyl- | 490.42; | ¹H NMR (400 MHz, CD₃OD) δ 7.74 (d, |
| | phenoxy)-5-fluoro-4- | 491.5 | J = 10.3 Hz, 1H), 7.21 (d, J = 5.8 Hz, |
| | (trifluoromethyl)phenyl]-2-methyl- | | 1H), 7.09 (q, J = 9.3 Hz, 1H), 6.76 (s, |
| | 3-(methylsulfonimidoyl)-1H- | | 1H), 6.64 (ddd, J = 9.3, 3.9, 2.1 Hz, 1H), |
| | pyridin-4-one | | 3.37 (s, 3H), 2.78 (s, 3H), 2.19 (d, J = 2.2 |
| | | | Hz, 3H). |
| 16 | ethyl 5-(2-amino-1-methoxy-2- | 537.51; | ¹H NMR (400 MHz, DMSO-d₆) δ 11.96 |
| | oxo-ethyl)-2-[2-(3,4-difluoro-2- | 538.6 | (br s, 1H), 8.53 (s, 1H), 8.03 (d, J = 8.0 |
| | methyl-phenoxy)-3-quinolyl]-6- | | Hz, 1H), 7.74 (t, J = 7.7 Hz, 1H), 7.66 (d, |
| | methyl-4-oxo-1H-pyridine-3- | | J = 8.4 Hz, 1H), 7.56 (t, J = 7.5 Hz, IH), |
| | carboxylate | | 7.40 (app q, J = 9.4 Hz, 1H), 7.31 (br s, |
| | | | 1H), 7.29 (br s, 1H), 7.14-7.06 (m, 1H), |
| | | | 5.25 (s, 1H), 3.92 (q, J = 7.0 Hz, 2H), |
| | | | 3.29 (s, 3H), 2.30 (s, 3H), 2.07 (d, J = 2.1 |
| | | | Hz, 3H), 0.73 (t, J = 7.1 Hz, 3H). |
| 17 | ethyl 5-(2-amino-1-methoxy-2- | 528.55; | ¹H NMR (400 MHz, DMSO-d₆) δ 11.77 |
| | oxo-ethyl)-2-[2-(4,4- | 529.5 | (br s, 1H), 8.12 (s, 1H), 7.81 (d, .J = 8.0 |
| | difluoroazepan-1-yl)-3-quinolyl]- | | Hz, 1H), 7.73-7.61 (m, 2H), 7.39-7.31 |
| | 6-methyl-4-oxo-1H-pyridine-3- | | (m, 1H), 7.28 (br s, 1H), 7.23 (br s, 1H), |
| | carboxylate | | 5.27 (br s, 1H), 3.95 (q, J = 7.1 Hz, 2H), |
| | | | 3.61 (app s, 2H), 3.55-3.37 (m, 2H), |
| | | | 3.26 (s, 3H), 2.26 (s, 3H) overlaps with |
| | | | 2.35-2.15 (m, 2H), 2.13-2.00 (m, 2H), |
| | | | 1.81-1.74 (m, 2H), 0.85 (t, J = 7.1 Hz, |
| | | | 3H). |
| 18 | methyl 2-[4-tert-butyl-2-(4,4- | 447.51; | ¹H NMR (400 MHz, DMSO-d₆) δ 11.54 |
| | difluorocyclohexyl)phenyl]-5- | 448.3 | (s, 1H), 7.39-7.29 (m, 2H), 7.13 (d, J = |
| | methoxy-6-methyl-4-oxo-1H- | | 8.0 Hz, 1H), 3.77 (s, 3H), 3.39 (s, 3H), |
| | pyridine-3-carboxylate | | 2.21 (s, 3H), 2.13-2.00 (m, 2H), 1.99- |
| | | | 1.91 (m, 1H), 1.90-1.58 (m, 6H), 1.30 |
| | | | (s, 9H). |
| 19 | ethyl 5-(2-amino-1-methyl-2-oxo- | 521.51; | ¹H NMR (400 MHz, DMSO-d₆) δ 11.87 |
| | ethyl)-2-[2-(3,4-difluoro-2-methyl- | 522.6 | (br s, 1H), 8.54 (s, 1H), 8.09 (d, J = 8.0 |
| | phenoxy)-3-quinolyl]-6-methyl-4- | | Hz, 1H), 7.84-7.75 (m, 1H), 7.72 (d, J = |
| | oxo-1H-pyridinc-3-carboxylatc | | 8.3 Hz, 1H), 7.66-7.58 (m, 1H), 7.46 |
| | | | (app q, J = 9.4 Hz, 1H), 7.19-7.11 (m, |
| | | | 1H), 7.06 (brs, 1H), 6.82 (br s, 1H), 3.96 |
| | | | (q, J = 6.9 Hz, 2H) overlaps with 3.92 (q, |
| | | | J = 7.2 Hz, 1H), 2.36 (s, 3H), 2.12 (d, J = |
| | | | 2.1 Hz, 3H), 1.34 (d, J = 7.2 Hz, 3H), |
| | | | 0.78 (t, J = 7.0 Hz, 3H). |
| 20 | ethyl 5-(2-amino-1-methyl-2-oxo- | 512.55; | ¹H NMR (400 MHz, DMSO-d₆) δ 11.60 |
| | ethyl)-2-[2-(4,4-difluoroazepan-1- | 513.7 | (br s, 1H), 8.06 (s, 1H), 7.80 (d, J = 8.0 |
| | yl)-3-quinolyl]-6-methyl-4-oxo- | | Hz, 1H), 7.71-7.61 (m, 2H), 7.34 (ddd, |
| | 1H-pyridine-3-carboxylate | | J = 8.0, 5.9, 2.1 Hz, 1H), 6.92 (br s, 1H), |
| | | | 6.75 (br s, 1H), 3.93 (q, J = 7.0 Hz, 3H) - |
| | | | two overlapping signals, 3.64-3.58 (m, |
| | | | 2H), 3.55-3.36 (m, 2H), 2.25 (s, 3H) |
| | | | overlaps with 2.25 (m, 2H), 2.12-1.96 |
| | | | (m, 2H), 1.84-1.73 (m, 2H), 1.24 (d, J = |
| | | | 7.1 Hz, 3H), 0.84 (t, J = 7.1 Hz, 3H). |
| 21 | 6-[2-(3,4-difluoro-2-methoxy- | 506.42; | ¹H NMR (400 MHz, DMSO-d₆) δ 7.97 |
| | phenoxy)-5-fluoro-4- | 507.6 | (d, J = 11.1 Hz, 1H), 7.24 (d, J = 6.0 Hz, |
| | (trifluoromethyl)phenyl]-2-methyl- | | 1H), 7.23-7.16 (m, 1H), 7.14-7.02 (m, |
| | 3-(methylsulfonimidoyl)-1H- | | 1H), 7.01-6.96 (m, 1H), 3.83 (d, J = 1.3 |
| | pyridin-4-one | | Hz, 3H), 3.42 (s, 3H), 2.74 (s, 3H). |
| 22 | 6-12-(3,4-difluoro-2-methoxy- | 506.42; | 1H NMR (400 MHz, DMSO-d₆) δ 7.97 |
| | phenoxy)-5-fluoro-4- | 507.6 | (d, J = 11.1 Hz, 1H), 7.24 (d, J = 6.0 Hz, |
| | (trifluoromethyl)phenyl]-2-methyl- | | 1H), 7.23-7.16 (m, 1H), 7.14-7.02 (m, |
| | 3-(methylsulfonimidoyl)-1H- | | 1H), 7.01-6.96 (m, 1H), 3.83 (d, J = 1.3 |
| | pyridin-4-one | | Hz, 3H), 3.42 (s, 3H), 2.74 (s, 3H). |
| 23 | ethyl 5-(2-amino-1-hydroxy-2- | 523.48; | ¹H NMR (400 MHz, DMSO-d₆) δ 12.04 |
| | oxo-ethyl)-2-[2-(3,4-difluoro-2- | 524.77 | (br s, 1H), 8.51 (s, 1H), 8.03 (d, J = 8.0 |
| | methyl-phenoxy)-3-quinolyl]-6- | | Hz, 1H), 7.74 (t, J = 7.5 Hz, 1H), 7.66 (d, |
| | methyl-4-oxo-1H-pyridine-3- | | J = 8.4 Hz, 1H), 7.57 (t, J = 7.5 Hz, 1H), |
| | carboxylate | | 7.44-7.37 (m, 1H) overlaps with 7.36 |
| | | | (br s, 1H), 7.28 (br s, 1H), 7.12-7.05 (m, |
| | | | 1H), 6.29 (app d, J = 6.9 Hz, 1H), 5.31 |
| | | | (app d, J = 6.7 Hz, 1H), 3.92 (q, J = 7.1 |
| | | | Hz, 2H), 2.35 (s, 3H), 2.07 (d, J = 2.1 Hz, |
| | | | 3H), 0.73 (t, J = 7.1 Hz, 3H). |
| 24 | ethyl 2-[2-(3,4-difluoro-2-methyl- | 536.52; | ¹H NMR (400 MHz, CD₃OD) δ 8.37 (s, |
| | phenoxy)-3-quinolyl]-5-(2- | 537.75 | 1H), 7.94 (d, J = 8.1 Hz, 1H), 7.76-7.66 |
| | methoxy-1-methyl-2-oxo-ethyl)-6- | | (m, 2H), 7.53 (ddd, J = 8.1, 5.8, 2.4 Hz, |
| | methyl-4-oxo-1H-pyridine-3- | | 1H), 7.17 (app q, J = 9.3 Hz, 1H), 7.07- |
| | carboxylate | | 7.00 (m, 1H), 3.99 (app q, J = 7.1 Hz, |
| | | | 2H), 3.87 (q, J = 6.9 Hz, 1H), 3.68 (s, |
| | | | 3H), 2.43 (s, 3H), 2.10 (d, .J = 2.2 Hz, |
| | | | 3H), 1.43 (d, J = 7.0 Hz, 3H), 0.77 (t, J = |
| | | | 7.1 Hz, 3H). |
| 25 | ethyl 5-(2-amino-1-hydroxy-2- | 514.52; | ¹H NMR (400 MHz, DMSO-d₆) δ 11.90 |
| | oxo-ethyl)-2-[2-(4,4- | 515.74 | (br s, 1H), 8.15 (s, 1H), 7.86 (d, J = 8.0 |
| | difluoroazepan-1-yl)-3-quinolyl]- | | Hz, 1H), 7.75-7.69 (m, 2H), 7.45-7.35 |
| | 6-methyl-4-oxo-1H-pyridine-3- | | (m, 2H), 7.31 (br s, 1H), 6.38 (br m, 1H), |
| | carboxylate | | 5.34 (app br s, 1H), 4.00 (q, J = 7.1 Hz, |
| | | | 2H), 3.67 (app s, 2H), 3.60-3.41 (br m, |
| | | | 2H), 2.37 (s, 3H) overlaps with 2.41- |
| | | | 2.29 (m, 2H), 2.16-2.03 (m, 2H), 1.89- |
| | | | 1.78 (m, 2H), 0.91 (t, J = 7.1 Hz, 3H). |
| 26 | ethyl 2-[2-(4,4-difluoroazepan-1- | 527.56; | ¹H NMR (400 MHz, DMSO-d₆) δ 11.69 |
| | yl)-3-quinolyl]-5-(2-methoxy-1- | 528.7 | (br s, 1H), 8.12 (br s, 1H), 7.82 (d, J = |
| | methyl-2-oxo-ethyl)-6-methyl-4- | | 8.0 Hz, 1H), 7.73-7.62 (m, 2H), 7.35 |
| | oxo-1H-pyridine-3-carboxylate | | (ddd, J = 8.1, 6.2, 1.8 Hz, 1H), 3.92 (q, |
| | | | J = 7.1 Hz, 2H), 3.80 (q, J = 6.9 Hz, 1H), |
| | | | 3.68-3.56 (m, 2H) overlaps with 3.54 (s, |
| | | | 3H) overlaps with 3.56-3.41 (m, 2H), |
| | | | 2.26 (s, 3H), 2.22-2.13 (m, 2H), 2.13- |
| | | | 1.98 (m, 2H), 1.83-1.69 (m, 2H), 1.27 |
| | | | (d, J = 7.0 Hz, 3H), 0.82 (t, J = 7.1 Hz, |
| | | | 3H). |
| 27 | ethyl 2-[2-(4,4-difluoroazepan-1- | 489.49; | ¹H NMR (400 MHz, CD₃OD) δ 8.04 (s, |
| | yl)-6-fluoro-3-quinolyl]-5- | 490.2 | 1H), 7.79 (dd, J = 8.8, 5.1 Hz, 1H), 7.53- |
| | methoxy-6-methyl-4-oxo-1H- | | 7.41 (m, 2H), 4.05 (q, J = 6.9 Hz, 2H), |
| | pyridine-3-carboxylate | | 3.90 (s, 3H), 3.69-3.59 (m, 2H), 3.55- |
| | | | 3.40 (m, 2H), 2.40 (s, 3H), 2.36-2.20 |
| | | | (m, 2H), 2.16-2.02 (m, 2H), 1.92-1.79 |
| | | | (m, 2H), 0.89 (t, J = 7.0 Hz, 3H). |
| 28 | ethyl 2-[2-(4,4-difluoroazepan-1- | 489.49; | ¹H NMR (400 MHz, CD₃OD) δ 8.03 (s, |
| | yl)-7-fluoro-3-quinolyl]-5- | 490.1 | 1H), 7.78 (dd, J = 8.9, 6.2 Hz, 1H), 7.35 |
| | methoxy-6-methyl-4-oxo-1H- | | (dd, J = 10.9, 2.6 Hz, 1H), 7.14 (td, J = |
| | pyridine-3-carboxylate | | 8.7, 2.6 Hz, 1H), 4.04 (q, J = 7.3 Hz, 2H), |
| | | | 3.87 (s, 3H), 3.70-3.63 (m, 2H), 3.48 |
| | | | (br. s., 2H), 2.38 (s, 3H), 2.36-2.25 (m, |
| | | | 2H), 2.12-1.99 (m, 2H), 1.90-1.82 (m, |
| | | | 2H), 0.89 (t, J = 7.1 Hz, 3H). |
| 29 | ethyl 2-[2-(4,4-difluoroazepan-1- | 461.5; | ¹H NMR (400 MHz, CDCl₃) δ 11.39 (s, |
| | yl)-6,7-dihydro-5H- | 462.6 | 1H), 7.33 (s, 1H), 4.16-4.05 (m, 1H), |
| | cyclopenta[b]pyridin-3-yl]-5- | | 4.03-3.94 (m, 1H), 3.92 (s, 3H), 3.24 (t, |
| | methoxy-6-methyl-4-oxo-1H- | | J = 5.4 Hz, 2H), 3.18-3.07 (m, 1H), |
| | pyridine-3-carboxylate | | 3.07-2.98 (m, 1H), 2.98-2.78 (m, 4H), 2.53 |
| | | | (s, 3H), 2.17-2.06 (m, 2H), 2.06-1.86 |
| | | | (m, 4H), 1.64-1.56 (m, 2H), 0.85 (t, J = |
| | | | 7.1 Hz, 3H). |
| 30 | ethyl 2-[2-(4,4-difluoroazepan-1- | 475.53; | ¹H NMR (400 MHz, CD₃OD) δ 7.80 (s, |
| | yl)-5,6,7,8-tetrahydroquinolin-3- | 476.7 | 1H), 4.24 (q, J = 7.1 Hz, 2H), 3.94 (s, |
| | yl]-5-methoxy-6-methyl-4-oxo- | | 3H), 3.64-3.55 (m, 2H), 3.50-3.42 (m, |
| | 1H-pyridine-3-carboxylate | | 2H), 3.00 (app t, J = 6.3 Hz, 2H), 2.74 |
| | | | (app t, J = 6.2 Hz, 2H), 2.61 (s, 3H), |
| | | | 2.34-2.18 (m, 2H), 2.18-2.04 (m, 2H), |
| | | | 2.00-1.91 (m, 2H), 1.91-1.80 (m, 4H), 1.15 |
| | | | (t, J = 7.1 Hz, 3H). |
| 31 | ethyl 2-[2-(3,4-difluoro-2-methyl- | 522.5; | ¹H NMR (400 MHz, CD₃OD) δ 8.39 (s, |
| | phenoxy)-3-quinolyl]-5-(2- | 523.61 | 1H), 7.94 (d, J = 8.1 Hz, 1H), 7.76-7.67 |
| | methoxy-2-oxo-ethyl)-6-methyl-4- | | (m, 2H), 7.53 (ddd, J = 8.2, 5.7, 2.4 Hz, |
| | oxo-1H-pyridine-3-carboxylate | | 1H), 7.17 (app q, J = 9.3 Hz, 1H), 7.07- |
| | | | 6.99 (m, 1H), 4.00 (q, J = 7.1 Hz, 2H), |
| | | | 3.71 (s, 3H), 3.69 (s, 2H), 2.42 (s, 3H), |
| | | | 2.09 (d, J = 2.2 Hz, 3H), 0.79 (t, J = 7.1 |
| | | | Hz, 3H). |
| 32 | ethyl 2-[2-(4,4-difluoroazepan-1- | 473.44; | ¹H NMR (400 MHz, CD₃OD) δ 8.55 (s, |
| | yl)-5-(trifluoromethyl)-3-pyridyl]- | 474.3 | 1H), 7.80 (s, 1H), 4.22-4.01 (m, 2H), |
| | 5,6-dimethyl-4-oxo-1H-pyridine- | | 3.71-3.60 (m, 2H), 3.27-3.06 (m, 2H), |
| | 3-carboxylate | | 2.51 (d, J = 3.4 Hz, 3H), 2.37-2.25 (m, |
| | | | 2H), 2.18 (d, J = 3.2 Hz, 3H), 2.06-1.92 |
| | | | (m, 2H), 1.90-1.79 (m, 2H), 1.01 (t, J = |
| | | | 7.1 Hz, 3H). |
| 33 | ethyl 6-[2-(3,4-difluoro-2-methyl- | 482.4; | ¹H NMR (400 MHz, DMSO-d₆) δ 11.66 |
| | phenoxy)-5-(trifluoromethyl)-3- | 483.2 | (s, 1H), 8.71 (d, J = 2.4 Hz, 1H), 8.49 (d, |
| | pyridyl]-2,5-dimethyl-4-oxo-1H- | | J = 2.5 Hz, 1H), 7.37 (q, J = 9.4 Hz, 1H), |
| | pyridine-3-carboxylate | | 7.09-6.96 (m, 1H), 4.23 (q, J = 7.1 Hz, |
| | | | 2H), 2.24 (s, 3H), 2.02 (d, J = 2.0 Hz, |
| | | | 3H), 1.77 (s, 3H), 1.26 (t, J = 7.1 Hz, |
| | | | 3H). |
| 34 | ethyl 2-[5-(4,4-difluoroazepan-1- | 473.44; | ¹H NMR (400 MHz, DMSO-d₆) δ 8.31 |
| | yl)-2-(trifluoromethyl)-4-pyridyl]- | 474.2 | (s, 1H), 7.25 (s, 1H), 3.81 (p, J = 6.9 Hz, |
| | 5,6-dimethyl-4-oxo-1H-pyridine- | | 2H), 3.38-3.31 (m, 4H), 2.15 (s, 3H), |
| | 3-carboxylate | | 2.04 (s, 5H), 1.87 (s, 3H), 1.74 (p, J = 5.9 |
| | | | Hz, 2H), 0.83 (t, J = 7.1 Hz, 3H). |
| 35 | ethyl 2-[2-(3,4-difluoro-2-methyl- | 521.47; | ¹H NMR (400 MHz, DMSO-d₆) δ 12.39 |
| | phenoxy)-3-quinolyl]-6-methyl-5- | 522.785 | (br s, 1H), 8.56 (br s, 1H), 8.05 (d, J = |
| | oxamoyl-4-oxo-1H-pyridine-3- | | 8.0 Hz, 1H), 7.89 (br s, 1H), 7.75 (t, J = |
| | carboxylate | | 7.7 Hz, 1H), 7.67 (d, J = 8.4 Hz, 1H), |
| | | | 7.58 (t, J = 7.5 Hz, 1H), 7.49 (br s, 1H), |
| | | | 7.40 (app q, J = 9.4 Hz, 1H), 7.13-7.06 |
| | | | (m, 1H), 3.93 (q, J = 7.1 Hz, 2H), 2.34 |
| | | | (s, 3H), 2.08 (d, J = 2.1 Hz, 3H), 0.74 (t, |
| | | | J = 7.1 Hz, 3H). |
| 36 | ethyl 6-[5-[2-methoxy-4- | 546.42; | ¹H NMR (400 MHz, DMSO-d₆) δ 11.58 |
| | (trifluoromethoxy)phenoxy]-2- | 547.1 | (s, 1H), 8.29 (s, 1H), 8.14 (s, 1H), 7.29 |
| | (trifluoromethyl)-4-pyridyl]-2,5- | | (d, J = 8.7 Hz, 1H), 7.21 (d, J = 2.8 Hz, |
| | dimethyl-4-oxo-1H-pyridine-3- | | 1H), 6.99 (d, .J = 8.5 Hz, 1H), 4.22 (q, |
| | carboxylate | | J = 7.6, 6.9 Hz, 2H), 3.77 (s, 3H), 2.21 (s, |
| | | | 3H), 1.71 (s, 3H), 1.26 (t, J = 7.1 Hz, 3H) |
| 37 | diethyl 2-[2-(4,4-difluoroazepan-1- | 545.5; | ¹H NMR (400 MHz, CD₃OD) δ 7.58 (s, |
| | yl)-5-methyl-6-(trifluoromethyl)- | 546.7 | 1H), 4.38 (q, J = 7.1 Hz, 2H), 4.10 (q, |
| | 3-pyridyl]-6-methyl-4-oxo-1H- | | J = 7.1 Hz, 2H), 3.61-3.54 (m, 2H), |
| | pyridine-3,5-dicarboxylate | | 3.37-3.31 (m, 2H) overlaps with CD₂HOD, |
| | | | 2.43 (s, 3H), 2.36 (q, J = 2.2 Hz, 3H), |
| | | | 2.34-2.21 (m, 2H), 2.07-1.92 (m, 2H), |
| | | | 1.91-1.81 (m, 2H), 1.37 (t, J = 7.1 Hz, |
| | | | 3H), 1.02 (t, J = 7.1 Hz, 3H). |
| 38 | diethyl 2-[2-(3,4-difluoro-2- | 554.46; | ¹H NMR (400 MHz, CD₃OD) δ 7.95 (s, |
| | methyl-phenoxy)-5-methyl-6- | 555.7 | 1H), 7.13 (app q, J = 9.3 Hz, 1H), 6.93 |
| | (trifluoromethyl)-3-pyridyl]-6- | | (app ddd, J = 9.1, 4.2, 2.1 Hz, 1H), 4.37 |
| | methyl-4-oxo-1H-pyridine-3,5- | | (q, J = 7.1 Hz, 2H), 4.10 (q, J = 7.1 Hz, |
| | dicarboxylate | | 2H), 2.47 (q, J = 2.2 Hz, 3H), 2.42 (s, |
| | | | 3H), 2.06 (d, J = 2.2 Hz, 3H), 1.37 (t, J = |
| | | | 7.1 Hz, 3H), 0.97 (t, .J = 7.1 Hz, 3H). |
| 39 | diethyl 2-[2-(4,4-difluoroazepan-1- | 513.53; | ¹H NMR (400 MHz, CD₃OD) δ 8.07 (s, |
| | yl)-3-quinolyl]-6-methyl-4-oxo- | 514.7 | 1H), 7.80-7.69 (m, 2H), 7.65 (t, J = 7.7 |
| | 1H-pyridine-3,5-dicarboxylate | | Hz, 1H), 7.34 (t, J = 7.4 Hz, 1H), 4.37 (q, |
| | | | J = 7.1 Hz, 2H), 4.04 (q, J = 7.1 Hz, 2H), |
| | | | 3.72-3.64 (m, 2H), 3.57-3.43 (m, 2H), |
| | | | 2.40 (s, 3H) overlaps with 2.37-2.26 (m, |
| | | | 2H), 2.13-1.99 (m, 2H), 1.92-1.80 (m, |
| | | | 2H), 1.37 (t, J = 7.1 Hz, 3H), 0.89 (t, J = |
| | | | 7.1 Hz, 3H). |
| 40 | diethyl 2-[2-(3,4-difluoro-2- | 522.5; | ¹H NMR (400 MHz, CD₃OD) δ 8.41 (s, |
| | methyl-phenoxy)-3-quinolyl]-6- | 523.996 | 1H), 7.94 (app d, J = 8.0 Hz, 1H), 7.77- |
| | methyl-4-oxo-1H-pyridine-3,5- | | 7.66 (m, 2H), 7.54 (ddd, J = 8.2, 6.2, 2.0 |
| | dicarboxylate | | Hz, 1H), 7.17 (app q, J = 9.3 Hz, 1H), |
| | | | 7.03 (app ddd, J = 9.1, 4.3, 2.0 Hz, 1H), |
| | | | 4.38 (q, J = 7.1 Hz, 2H), 4.01 (q, J = 7.1 |
| | | | Hz, 2H), 2.43 (s, 3H), 2.10 (d, J = 2.2 Hz, |
| | | | 3H), 1.37 (t, J = 7.1 Hz, 3H), 0.81 (t, J = |
| | | | 7.1 Hz, 3H). |
| 41 | methyl 2-[2-(3,4-difluoro-2- | 498.4; | ¹H NMR (400 MHz, CD₃OD) δ 7.96 (s, |
| | methyl-phenoxy)-5-methyl-6- | 499.6 | 1H), 7.13 (app q, J = 9.3 Hz, 1H), 6.90 |
| | (trifluoromethyl)-3-pyridyl]-5- | | (app ddd, J = 9.2, 4.2, 2.1 Hz, 1H), 3.91 |
| | methoxy-6-methyl-4-oxo-1H- | | (s, 3H), 3.68 (s, 3H), 2.51-2.45 (m, 6H), |
| | pyridine-3-carboxylate | | 2.03 (d, J = 2.2 Hz, 3H). |
| 42 | ethyl 6-[2-(3,4-difluoro-2-methyl- | 496.43; | ¹H NMR (400 MHz, CD₃OD) δ 8.47 (s, |
| | phenoxy)-4-methyl-5- | 497.4 | 1H), 7.14 (q, J = 9.3 Hz, 1H), 6.87 (m, |
| | (trifluoromethyl)-3-pyridyl]-2,5- | | 1H), 4.37 (q, J = 7.1 Hz, 2H), 2.40 (s, |
| | dimethyl-4-oxo-1H-pyridine-3- | | 3H), 2.38-2.33 (m, 3H), 2.03 (d, J = 2.2 |
| | carboxylate | | Hz, 3H), 1.88 (s, 3H), 1.37 (t, J = 7.1 Hz, |
| | | | 3H). |
| 43 | ethyl 6-[2-(3,4-difluoro-2-methyl- | 496.43; | ¹H NMR (400 MHz, CD₃OD) δ 8.46 (s, |
| | phenoxy)-4-methyl-5- | 497.3 | 1H), 7.13 (q, J = 9.3 Hz, 1H), 6.86 (m, |
| | (trifluoromethyl)-3-pyridyl]-2,5- | | 1H), 4.37 (q, J = 7.1 Hz, 2H), 2.40 (s, |
| | dimethyl-4-oxo-1H-pyridine-3- | | 3H), 2.36 (d, J = 1.5 Hz, 3H), 2.03 (d, |
| | carboxylate | | J = 2.2 Hz, 3H), 1.88 (s, 3H), 1.37 (t, J = |
| | | | 7.1 Hz, 3H). |
| 44 | methyl 2-[2-(4,4-difluoroazepan-1- | 489.44; | ¹H NMR (400 MHz, CD₃OD) δ 7.51 (s, |
| | yl)-5-methyl-6-(trifluoromethyl)- | 490.5 | 1H), 3.85 (s, 3H), 3.63 (s, 3H), 3.58- |
| | 3-pyridyl]-5-methoxy-6-methyl-4- | | 3.50 (m, 2H), 3.36-3.31 (m, 2H) |
| | oxo-1H-pyridine-3-carboxylate | | overlaps with CD₂HOD, 2.38-2.32 (m, |
| | | | 3H) overlaps with 2.38-2.32 (s, 3H), |
| | | | 2.30-2.18 (m, 2H), 2.07-1.92 (m, 2H), |
| | | | 1.88-1.77 (m, 2H). |
| 45 | ethyl 6-[2-(3,4-difluoro-2-methyl- | 496.43; | ¹H NMR (400 MHz, CD₃OD) δ 8.48 (s, |
| | phenoxy)-4-methyl-5- | 497.4 | 1H), 7.14 (q, J = 9.3 Hz, 1H), 6.93-6.82 |
| | (trifluoromethyl)-3-pyridyl]-2,5- | | (m, 1H), 4.40 (q, J = 7.1 Hz, 2H), 2.47 |
| | dimethyl-4-oxo-1H-pyridine-3- | | (s, 3H), 2.37 (d, J = 1.4 Hz, 3H), 2.03 (d, |
| | carboxylate | | J = 2.1 Hz, 3H), 1.92 (s, 3H), 1.38 (t, J = |
| | | | 7.1Hz, 3H). |
| 46 | ethyl 6-[2-(3,4-difluoro-2-methyl- | 482.4; | ¹H NMR (400 MHz, DMSO-d₆) δ 11.86 |
| | phenoxy)-4-methyl-5- | 483.2 | (s, 1H), 8.55 (s, 1H), 7.35 (q, J = 9.4 Hz, |
| | (trifluoromethyl)-3-pyridyl]-2- | | 1H), 7.12-6.97 (m, 1H), 6.33 (s, 1H), |
| | methyl-4-oxo-1H-pyridine-3- | | 4.36-4.14 (m, 2H), 2.41-2.31 (m, 3H), |
| | carboxylate | | 2.27 (s, 3H), 2.02 (d, J = 2.1 Hz, 3H), |
| | | | 1.27 (t, J = 7.1 Hz, 3H). |
| 47 | methyl 2-[2-(4,4-difluoroazepan-1- | 457.47; | ¹H NMR (400 MHz, CD₃OD) δ 8.02 (s, |
| | yl)-3-quinolyl]-5-methoxy-6- | 458.55 | 1H), 7.78-7.69 (m, 2H), 7.69-7.57 (m, |
| | methyl-4-oxo-1H-pyridine-3- | | 1H), 7.39-7.27 (m, 1H), 3.87 (s, 3H), |
| | carboxylate | | 3.67-3.60 (m, 2H), 3.58 (s, 3H), 3.53- |
| | | | 3.39 (m, 2H), 2.37 (s, 3H), 2.34-2.18 |
| | | | (m, 2H), 2.13-1.97 (m, 2H), 1.89-1.77 |
| | | | (m, 2H). |
| 48 | methyl 2-[2-(3,4-difluoro-2- | 517.37; | ¹H NMR (500 MHz, DMSO-d₆) δ 11.87 |
| | methoxy-phenoxy)-5-fluoro-4- | 518.4 | (s, 1H), 7.81-7.75 (m, 1H), 7.25-7.15 |
| | (trifluoromethyl)phenyl]-5- | | (m, 2H), 6.86 (s, 1H), 3.79 (s, 3H), 3.74 |
| | methoxy-6-methyl-4-oxo-1H- | | (s, 3H), 3.53 (s, 3H), 2.23 (s, 3H). |
| | pyridine-3-carboxylate | | |
| 49 | methyl 2-12-(3,4-difluoro-2- | 466.43; | ¹H NMR (400 MHz, CD₃OD) δ 8.43 (s, |
| | methyl-phenoxy)-3-quinolyl]-5- | 467.372 | 1H), 7.96 (app d, J = 8.1 Hz, 1H), 7.79- |
| | methoxy-6-methyl-4-oxo-1H- | | 7.67 (m, 2H), 7.55 (ddd, J = 8.1, 6.5, 1.6 |
| | pyridine-3-carboxylate | | Hz, 1H), 7.18 (app q, J = 9.3 Hz, 1H), |
| | | | 6.99 (app ddd, J = 9.1, 4.2, 2.0 Hz, 1H), |
| | | | 3.93 (s, 3H), 3.60 (s, 3H), 2.52 (s, 3H), |
| | | | 2.07 (d, J = 2.2 Hz, 3H). |
| 50 | ethyl 6-[2-(3,4-difluoro-2-methyl- | 496.43; | ¹H NMR (400 MHz, CD₃OD) δ 8.05 (s, |
| | phenoxy)-5-methyl-6- | 497.2 | 1H), 7.13 (q, J = 9.3 Hz, 1H), 6.99-6.85 |
| | (trifluoromethyl)-3-pyridyl]-2,5- | | (m, 1H), 4.46 (q, J = 7.1 Hz, 2H), 2.60 |
| | dimethyl-4-oxo-1H-pyridine-3- | | (s, 3H), 2.57-2.46 (m, 3H), 2.06 (s, 3H), |
| | carboxylate | | 2.04 (d, J = 2.3 Hz, 3H), 1.41 (t, J = 7.1 |
| | | | Hz, 3H). |
| 51 | ethyl 2-[2-(3,4-difluoro-2-methyl- | 482.4; | ¹H NMR (400 MHz, CD₃OD) δ 8.59-8.48 |
| | phenoxy)-5-(trifluoromethyl)-3- | 483.2 | (m, 1H), 8.30-8.19 (m, 1H), 7.22- |
| | pyridyl]-5,6-dimethyl-4-oxo-1H- | | 7.07 (m, 1H), 6.97-6.81 (m, 1H), 4.14 |
| | pyridine-3-carboxylate | | (q, J = 7.1 Hz, 2H), 2.49 (s, 3H), 2.16 (s, |
| | | | 3H), 2.04 (s, 3H), 0.96 (t, J = 7.1 Hz, |
| | | | 3H). |
| 52 | ethyl 6-[2-(4,4-difluoroazepan-1- | 455.5; | ¹H NMR (400 MHz, CD₃OD) δ 8.63 (s, |
| | yl)-3-quinolyl]-2,5-dimethyl-4- | 456.3 | 1H), 8.10 (d, J = 8.5 Hz, 1H), 8.07-7.90 |
| | oxo-1H-pyridine-3-carboxylate | | (m, 2H), 7.70-7.55 (m, 1H), 4.58 (q, |
| | | | J = 7.1 Hz, 2H), 3.84-3.70 (m, 2H), |
| | | | 3.56-3.45 (m, 2H), 2.87 (s, 3H), 2.46-2.30 |
| | | | (m, 2H), 2.22 (s, 3H), 2.20-2.08 (m, |
| | | | 2H), 1.98-1.86 (m, 2H), 1.48 (t, J = 7.1 |
| | | | Hz, 3H). |
| 53 | ethyl 2-[2-(3,4-difluoro-2-methyl- | 512.43; | ¹H NMR (400 MHz, CD₃OD) δ 7.92 (s, |
| | phenoxy)-5-methyl-6- | 513.7 | 1H), 7.12 (app q, J = 9.3 Hz, 1H), 6.91 |
| | (trifluoromethyl)-3-pyridyl]-5- | | (app ddd, J = 9.1, 4.2, 2.1 Hz, 1H), 4.11 |
| | methoxy-6-methyl-4-oxo-1H- | | (q, J = 7.1 Hz, 2H), 3.88 (s, 3H), 2.47 (q, |
| | pyridine-3-carboxylate | | J = 2.2 Hz, 3H), 2.44 (s, 3H), 2.04 (d, |
| | | | J = 2.3 Hz, 3H), 0.95 (t, J = 7.1 Hz, 3H). |
| 54 | 6-[5-chloro-2-(4,4-difluoroazepan- | 435.82; | ¹H NMR (400 MHz, DMSO-d₆) δ 8.38 |
| | 1-yl)-6-(trifluoromethyl)-3- | 436.1 | (br. s., 1H), 7.01 (s, 1H), 6.76-6.49 (m, |
| | pyridyl]-2,3-dimethyl-1H-pyridin- | | 1H), 3.81-3.73 (m, 2H), 3.73-3.65 (m, |
| | 4-one | | 2H), 2.41-2.20 (m, 5H), 2.18-1.98 (m, |
| | | | 5H), 1.94-1.84 (m, 2H). |
| 55 | 6-[2-(3,4-difluoro-2-methoxy- | 430.3; | ¹H NMR (500 MHz, DMSO-d₆) δ 12.65 |
| | phenoxy)-5-fluoro-4- | 431.4 | (s, 1H), 7.98 (d, J = 11.3 Hz, 1H), 7.26- |
| | (trifluoromethyl)phenyl]-2-methyl- | | 7.15 (m, 2H), 6.97 (ddd, J = 9.4, 5.0, 2.3 |
| | 1H-pyrimidin-4-one | | Hz, 1H), 6.77 (s, 1H), 3.84 (d, J = 1.2 Hz, |
| | | | 3H), 2.34 (s, 3H). |
| 56 | methyl 2-[5-(3,4-difluoro-2- | 500.37; | ¹H NMR (500 MHz, DMSO-d₆) δ 11.91 |
| | methoxy-phenoxy)-2- | 500.9 | (s, 1H), 8.36 (s, 1H), 8.07 (s, 1H), 7.24 |
| | (trifluoromethyl)-4-pyridyl]-5- | | (q, J = 9.3 Hz, 1H), 6.99-6.95 (m, 1H), |
| | methoxy-6-methyl-4-oxo-1H- | | 3.80 (s, 3H), 3.75 (s, 3H), 3.54 (s, 3H), |
| | pyridine-3-carboxylate | | 2.27 (s, 3H). |
| 57 | 2-[2-(3,4-difluoro-2-methoxy- | 430.3; | ¹H NMR (500 MHz, DMSO-d₆) δ 12.72 |
| | phenoxy)-5-fluoro-4- | 431.0 | (s, 1H), 7.90 (d, J = 10.5 Hz, 1H), 7.30 |
| | (trifluoromethyl)phenyl]-6-methyl- | | (s, 1H), 7.20 (d, J = 9.4 Hz, 1H), 7.00 (s, |
| | 1H-pyrimidin-4-one | | 1H), 6.22 (s, 1H), 3.82 (d, J = 1.1 Hz, |
| | | | 3H), 2.20 (s, 3H). |
| 58 | ethyl 2-[5-(3,4-difluoro-2- | 514.4; | ¹H NMR (500 MHz, DMSO-d₆) δ 11.93 |
| | methoxy-phenoxy)-2- | 515.1 | (s, 1H), 8.36 (s, 1H), 8.08 (s, 1H), 7.27- |
| | (trifluoromethyl)-4-pyridyl]-5- | | 7.22 (m, 1H), 7.00-6.97 (m, 1H), 4.01- |
| | methoxy-6-methyl-4-oxo-1H- | | 3.97 (m, 2H), 3.82 (s, 3H), 3.76 (s, 3H), |
| | pyridine-3-carboxylate | | 2.26 (s, 3H), 0.85 (t, J = 7.1 Hz, 3H). |
| 59 | ethyl 6-[5-(3,4-difluoro-2- | 498.4; | ¹H NMR (500 MHz, DMSO-d₆) δ 11.63 |
| | methoxy-phenoxy)-2- | 499.5 | (s, 1H), 8.41 (s, 1H), 8.16 (s, 1H), 7.23 |
| | (trifluoromethyl)-4-pyridyl]-2,5- | | (q, J = 9.4 Hz, 1H), 7.11-7.04 (m, 1H), |
| | dimethyl-4-oxo-1H-pyridine-3- | | 4.21 (q, J = 7.1 Hz, 2H), 3.81 (d, J = 1.5 |
| | carboxylate | | Hz, 3H), 2.22 (s, 3H), 1.72 (s, 3H), 1.25 |
| | | | (t, J = 7.1 Hz, 3H). |
| 60 | ethyl 6-[2-(4,4-difluoroazepan-1- | 484.54; | ¹H NMR (400 MHz, CD₃OD) δ 8.69 (s, |
| | yl)-3-quinolyl]-5- | 485.4 | 1H), 8.11 (d, J = 8.3 Hz, 1H), 8.05-7.93 |
| | (dimethylamino)-2-methyl-4-oxo- | | (m, 2H), 7.64 (t, J = 7.5 Hz, 1H), 4.46 (q, |
| | 1H-pyridine-3-carboxylate | | J = 7.1 Hz, 2H), 3.87 (d, J = 7.4 Hz, 2H), |
| | | | 3.69 (s, 2H), 3.27-3.05 (m, 6H), 2.58 (s, |
| | | | 3H), 2.46 (d, J = 9.9 Hz, 2H), 2.18 (d, |
| | | | J = 16.6 Hz, 2H), 2.04 (d, J = 6.3 Hz, |
| | | | 2H), 1.41 (t, J = 7.1 Hz, 3H). |
| 61 | ethyl 6-[2-(3,4-difluoro-2-methyl- | 493.5; | ¹H NMR (400 MHz, DMSO-d₆) δ 12.35 |
| | phenoxy)-3-quinolyl]-5- | 494.3 | (s, 1H), 8.68 (s, 1H), 8.05 (dd, J = 8.1, |
| | (dimethylamino)-2-methyl-4-oxo- | | 1.5 Hz, 1H), 7.82-7.74 (m, 1H), 7.69 (d, |
| | 1H-pyridine-3-carboxylate | | J = 8.3 Hz, 1H), 7.64-7.56 (m, 1H), 7.39 |
| | | | (q, J = 9.4 Hz, 1H), 7.16-7.08 (m, 1H), |
| | | | 4.28 (q, J = 7.1 Hz, 2H), 2.96 (s, 6H), |
| | | | 2.29 (s, 3H), 2.08 (d, J = 2.1 Hz, 3H), |
| | | | 1.29 (t, J = 7.1 Hz, 3H). |
| 62 | ethyl 2-[2-(3,4-difluoro-2- | 531.4; | ¹H NMR (500 MHz, DMSO-d₆) δ 11.82 |
| | methoxy-phenoxy)-5-fluoro-4- | 532.5 | (s, 1H), 7.74 (s, 1H), 7.23-7.17 (m, 2H), |
| | (trifluoromethyl)phenyl]-5- | | 6.86 (s, 1H), 4.00 (q, J = 7.1 Hz, 2H), |
| | methoxy-6-methyl-4-oxo-1H- | | 3.80 (s, 3H), 3.74 (s, 3H), 2.23 (s, 3H), |
| | pyridine-3-carboxylate | | 0.90 (t, J = 7.1 Hz, 3H). |
| 63 | ethyl 6-[2-(4,4-difluoroazepan-1- | 487.46; | |
| | yl)-5-methyl-6-(trifluoromethyl)- | 488.3 | |
| | 3-pyridyl]-2,5-dimethyl-4-oxo-1H- | | |
| | pyridine-3-carboxylate | | |
| 64 | ethyl 6-[2-(3,4-difluoro-2-methyl- | 464.46; | ¹H NMR (400 MHz, CD₃OD) δ 8.47 (s, |
| | phenoxy)-3-quinolyl]-2,5- | 465.2 | 1H), 8.01-7.95 (m, 1H), 7.78-7.69 (m, |
| | dimethyl-4-oxo-1H-pyridine-3- | | 2H), 7.60-7.52 (m, 1H), 7.17 (q, J = 9.2 |
| | carboxylate | | Hz, 1H), 7.03-6.95 (m, 1H), 4.41 (q, J = |
| | | | 7.1 Hz, 2H), 2.48 (s, 3H), 2.07 (d, J = |
| | | | 2.2 Hz, 3H), 2.01 (s, 3H), 1.39 (t, J = 7.1 |
| | | | Hz, 3H). |
| 65 | 6-[2-(3,4-difluoro-2-methoxy- | 443.34; | ¹H NMR (500 MHz, DMSO-d₆) δ 10.49 |
| | phenoxy)-5-fluoro-4- | 444.0 | (s, 1H), 7.96 (d, J = 11.5 Hz, 1H), 7.36 |
| | (trifluoromethyl)phenyl]-2,3- | | (s, 1H), 7.25-7.12 (m, 2H), 6.93 (s, 1H), |
| | dimethyl-1H-pyridin-4-one | | 3.82 (s, 3H), 2.42 (s, 3H), 2.14-2.02 (m, |
| | | | 3H). |
| 66 | 2-[2-(4,4-difluoroazepan-1-yl)-3- | 412.48; | ¹H NMR (400 MHz, CD₃OD) δ 8.73 (d, |
| | quinolyl]-3-(dimethylamino)-6- | 413.4 | J = 2.2 Hz, 1H), 8.19 (d, J = 8.6 Hz, 1H), |
| | methyl-1H-pyridin-4-one | | 8.08-7.94 (m, 2H), 7.68 (td, J = 7.6, 7.2, |
| | | | 1.0 Hz, 1H), 7.16 (s, 1H), 3.89-3.77 (m, |
| | | | 2H), 3.65 (t, J = 5.5 Hz, 2H), 2.72 (s, |
| | | | 3H), 2.66 (d, J = 5.3 Hz, 6H), 2.52-2.34 |
| | | | (m, 2H), 2.18 (td, J = 14.4, 13.8, 6.6 Hz, |
| | | | 2H), 2.02 (d, J = 8.0 Hz, 2H). |
| 67 | ethyl 2-[2-(4,4-difluoroazepan-1- | 457.47; | ¹H NMR (400 MHz, CD₃OD) δ 8.46 (s, |
| | yl)-3-quinolyl]-5-hydroxy-6- | 458.5 | 1H), 8.05 (d, J = 8.5 Hz, 1H), 7.97-7.86 |
| | methyl-4-oxo-1H-pyridine-3- | | (m, 2H), 7.60 (t, J = 7.6 Hz, 1H), 4.17 (q, |
| | carboxylate | | J = 7.1 Hz, 2H), 3.82-3.71 (m, 2H), 3.68- |
| | | | 3.55 (m, 2H), 2.55 (s, 3H), 2.39-2.25 |
| | | | (m, 2H), 2.21-2.07 (m, 2H), 1.99-1.86 |
| | | | (m, 2H), 0.95 (t, J = 7.1 Hz, 3H). |
| 68 | ethyl 2-[2-(3,4-difluoro-2-methyl- | 496.43; | ¹H NMR (400 MHz, CD₃OD) δ 7.90 (s, |
| | phenoxy)-5-methyl-6- | 497.2 | 1H), 7.11 (q, J = 9.3 Hz, 1H), 6.98-6.81 |
| | (trifluoromethyl)-3-pyridyl]-5,6- | | (m, 1H), 4.10 (q, J = 7.1 Hz, 2H), 2.47 |
| | | | (q, J = 2.2 Hz, 3H), 2.44 (s, 3H), 2.12 (s, |
| | dimethyl-4-oxo-1H-pyridine-3- | | 3H), 2.03 (d, J = 2.3 Hz, 3H), 0.94 (t, J = |
| | carboxylate | | 7.1 Hz, 3H). |
| 69 | 2-[2-(3,4-difluoro-2-methoxy- | 457.37; | ¹H NMR (500 MHz, Acetonitrile-d3) δ |
| | phenoxy)-5-fluoro-4- | 458.0 | 7.42 (d, J = 10.2 Hz, 1H), 7.16 (d, J = 5.9 |
| | (trifluoromethyl)phenyl]-3,5,6- | | Hz, 1H), 6.96 (ddd, J = 10.0, 9.4, 8.4 Hz, |
| | trimethyl-1H-pyridin-4-one | | 1H), 6.81 (ddd, J = 9.3, 5.0, 2.4 Hz, 1H), |
| | | | 3.78 (d, J = 1.5 Hz, 3H), 2.26 (s, 3H), |
| | | | 1.96 (s, 3H), 1.82 (s, 3H). |
| 70 | ethyl 2-[2-(4,4-difluoroazepan-1- | 489.44; | ¹H NMR (400 MHz, CD₃OD) δ 7.51 (s, |
| | yl)-5-methyl-6-(trifluoromethyl)- | 490.5 | 1H), 4.08 (q, J = 7.1 Hz, 2H), 3.56-3.49 |
| | 3-pyridyl]-5-hydroxy-6-methyl-4- | | (m, 2H), 3.29-3.22 (m, 2H) overlaps |
| | oxo-1H-pyridine-3-carboxylate | | with CHD₂OD, 2.38 (s, 3H), 2.36 (q, J = |
| | | | 2.2 Hz, 3H), 2.32-2.17 (m, 2H), 2.06- |
| | | | 1.91 (m, 2H), 1.86-1.75 (m, 2H), 0.98 |
| | | | (t, J = 7.1 Hz, 3H). |
| 71 | ethyl 2-[2-(3,4-difluoro-2-methyl- | 466.43; | ¹H NMR (400 MHz, CD₃OD) δ 8.36 (s, |
| | phenoxy)-3-quinolyl]-5-hydroxy- | 467.5 | 1H), 7.94 (d, J = 8.1 Hz, 1H), 7.76-7.66 |
| | 6-methyl-4-oxo-1H-pyridine-3- | | (m, 2H), 7.53 (ddd, J = 8.2, 5.4, 2.8 Hz, |
| | carboxylate | | 1H), 7.16 (app q, J = 9.3 Hz, 1H), 7.00 |
| | | | (ddd, J = 9.1, 4.3, 2.0 Hz, 1H), 4.01 (q, |
| | | | J = 7.1 Hz, 2H), 2.41 (s, 3H), 2.08 (d, J = |
| | | | 2.2 Hz, 3H), 0.77 (t, J = 7.1 Hz, 3H). |
| 72 | ethyl 2-[5-[2-methoxy-4- | 546.42; | ¹H NMR (400 MHz, CDCl₃) δ 11.87 (s, |
| | (trifluoromethoxy)phenoxy]-2- | 547.05 | 1H), 8.18 (s, 1H), 7.77 (s, 1H), 6.91 (d, |
| | (trifluoromethyl)-4-pyridyl]-5,6- | | J = 8.2 Hz, 1H), 6.75 (d, J = 8.3 Hz, 2H), |
| | dimethyl-4-oxo-1H-pyridine-3- | | 4.19 (q, J = 7.2 Hz, 2H), 3.77 (s, 3H), |
| | carboxylate | | 2.53 (s, 3H), 2.22 (s, 3H), 0.91 (t, J = 7.2 |
| | | | Hz, 3H) |
| 73 | ethyl 2-[2-(4,4-difluoroazepan-1- | 471.5; | ¹H NMR (400 MHz, CD₃OD) δ 8.03 (s, |
| | yl)-3-quinolyl]-5-methoxy-6- | 472.6 | 1H), 7.78-7.69 (m, 2H), 7.64 (ddd, J = |
| | | | 8.4, 6.9, 1.5 Hz, 1H), 7.34 (ddd, J = 8.1, |
| | methyl-4-oxo-1H-pyridine-3- | | 6.9, 1.2 Hz, 1H), 4.03 (q, J = 7.1 Hz, 2H), |
| | carboxylate | | 3.87 (s, 3H), 3.69-3.56 (m, 2H), 3.48 (s, |
| | | | 2H), 2.38 (s, 3H), 2.34-2.18 (m, 2H), |
| | | | 2.14-1.98 (m, 2H), 1.92-1.75 (m, 2H), |
| | | | 0.87 (t, J = 7.1 Hz, 3H). |
| 74 | ethyl 5-cyano-2-[2-(3,4-difluoro-2- | 475.44; | ¹H NMR (400 MHz, CD₃OD) δ 8.44 (s, |
| | methyl-phenoxy)-3-quinolyl]-6- | 476.5 | 1H), 7.95 (d, J = 7.4 Hz, 1H), 7.79-7.67 |
| | methyl-4-oxo-1H-pyridine-3- | | (m, 2H), 7.55 (ddd, J = 8.2, 6.6, 1.6 Hz, |
| | carboxylate | | 1H), 7.18 (q, J = 9.3 Hz, 1H), 7.06-7.00 |
| | | | (m, 1H), 4.02 (q, J = 7.1 Hz, 2H), 2.63 |
| | | | (s, 3H), 2.10 (d, .J = 2.2 Hz, 3H), 0.82 (t, |
| | | | J = 7.1 Hz, 3H). |
| 75 | ethyl 2-[2-(4,4-difluoroazepan-1- | 455.5; | ¹H NMR (400 MHz, CD₃OD) δ 8.65 (s, |
| | yl)-3-quinolyl]-5,6-dimethyl-4- | 456.5 | 1H), 8.19 (d, J = 8.8 Hz, 1H), 8.04-7.96 |
| | oxo-1H-pyridine-3-carboxylate | | (m, 2H), 7.68 (t, J = 7.6 Hz, 1H), 4.28 (q, |
| | | | J = 7.1 Hz, 2H), 3.83 (d, J = 6.9 Hz, 2H), |
| | | | 3.68 (br s, 2H), 2.79 (s, 3H), 2.46-2.33 |
| | | | (m, 3H), 2.23-2.10 (m, 2H), 2.03-1.94 |
| | | | (m, 2H), 0.96 (t, J = 7.1 Hz, 3H). |
| 76 | 2-[2-(3,4-difluoro-2-methyl- | 421.44; | ¹H NMR (400 MHz, DMSO-d₆) δ 8.66 |
| | phenoxy)-3-quinolyl]-3- | 422.3 | (s, 1H), 8.06 (d, J = 7.7 Hz, 1H), 7.82- |
| | (dimethylamino)-6-methyl-1H- | | 7.74 (m, 1H), 7.70 (d, J = 8.3 Hz, 1H), |
| | pyridin-4-one | | 7.61 (t, J = 7.5 Hz, 1H), 7.39 (q, J = 9.3 |
| | | | Hz, 1H), 7.12 (dd, J = 9.6, 3.9 Hz, 1H), |
| | | | 7.00 (s, 1H), 2.72 (s, 6H), 2.53 (s, 3H), |
| | | | 2.03 (d, J = 2.1 Hz, 3H |
| 77 | ethyl 2-[2-(4,4-difluoroazepan-1- | 503.46; | ¹H NMR (400 MHz, CD₃OD) δ 7.51 (s, |
| | yl)-5-methyl-6-(trifluoromethyl)- | 504.6 | 1H), 4.08 (q, J = 7.1 Hz, 2H), 3.85 (s, |
| | 3-pyridyl]-5-methoxy-6-methyl-4- | | 3H), 3.60-3.51 (m, 2H), 3.37-3.31 (m, |
| | oxo-1H-pyridine-3-carboxylate | | 2H) overlaps with CD2HOD, 2.36 (s, |
| | | | 3H) overlaps with 2.35-2.33 (m, 3H), |
| | | | 2.32-2.17 (m, 2H), 2.07-1.92 (m, 2H), |
| | | | 1.88-1.77 (m, 2H), 1.00 (t, J = 7.1 Hz, |
| | | | 3H). |
| 78 | ethyl 2-[2-(3,4-difluoro-2-methyl- | 480.46; | ¹H NMR (400 MHz, CD₃OD) δ 8.37 (s, |
| | phenoxy)-3-quinolyl]-5-methoxy- | 481.404 | 1H), 7.94 (app d, J = 8.2 Hz, 1H), 7.76- |
| | 6-methyl-4-oxo-1H-pyridine-3- | | 7.66 (m, 2H), 7.53 (ddd, J = 8.1, 6.0, 2.1 |
| | carboxylate | | Hz, 1H), 7.17 (app q, J = 9.2 Hz, 1H), |
| | | | 7.01 (ddd, J = 9.1, 4.2, 2.0 Hz, 1H), 4.02 |
| | | | (q, J = 7.1 Hz, 2H), 3.89 (s, 3H), 2.42 (s, |
| | | | 3H), 2.09 (d, J = 2.2 Hz, 3H), 0.78 (t, J = |
| | | | 7.1 Hz, 3H). |
| 79 | methyl 2-[2-(3,4-difluoro-2- | 487.35; | ¹H NMR (400 MHz, DMSO-d₆) δ 11.67 |
| | methoxy-phenoxy)-5-fluoro-4- | 488.4 | (s, 1H), 7.71-7.66 (m, 1H), 7.24-7.12 |
| | (trifluoromethyl)phenyl]-6-methyl- | | (m, 2H), 6.85-6.80 (m, 1H), 6.31 (s, |
| | 4-oxo-1H-pyridine-3-carboxylate | | 1H), 3.77 (s, 3H), 3.54 (s, 3H), 2.26 (s, |
| | | | 3H). |
| 80 | ethyl 2-[5-(3,4-difluoro-2- | 498.4; | ¹H NMR (400 MHz, CDCl₃) δ 11.82 (s, |
| | methoxy-phenoxy)-2- | 499.1 | 1H), 8.16 (s, 1H), 7.76 (d, J = 0.5 Hz, |
| | (trifluoromethyl)-4-pyridyl]-5,6- | | 1H), 6.82 (td, J = 9.4, 8.0 Hz, 1H), 6.67 |
| | dimethyl-4-oxo-1H-pyridine-3- | | (ddd, J = 9.3, 4.9, 2.4 Hz, 1H), 4.16 (dq, |
| | carboxylate | | J = 16.1, 6.8 Hz, 2H), 3.85 (d, J = 1.9 Hz, |
| | | | 3H), 2.55 (d, J = 0.6 Hz, 3H), 2.33-2.21 |
| | | | (m, 3H), 0.90 (t, J = 7.2 Hz, 3H). |
| 81 | ethyl 2-[2-(3,4-difluoro-2- | 501.38; | ¹H NMR (400 MHz, DMSO-d₆) δ 11.68 |
| | methoxy-phenoxy)-5-fluoro-4- | 502.4 | (s, 1H), 7.69-7.64 (m, 1H), 7.24-7.12 |
| | (trifluoromethyl)phenyl]-6-methyl- | | (m, 2H), 6.86-6.81 (m, 1H), 6.34 (s, |
| | 4-oxo-1H-pyridine-3-carboxylate | | 1H), 4.00 (q, J = 7.1 Hz, 2H), 3.78 (s, |
| | | | 3H), 2.26 (s, 3H), 0.90 (t, J = 7.1 Hz, |
| | | | 3H). |
| 82 | 6-[2-(3,4-difluoro-2-methoxy- | 468.35; | ¹H NMR (400 MHz, CD₃OD) δ 7.58 (d, |
| | phenoxy)-5-fluoro-4- | 469.4 | J = 10.0 Hz, 1H), 7.18 (d, J = 5.8 Hz, |
| | (trifluoromethyl)phenyl]-2,5- | | 1H), 7.06-6.95 (m, 1H), 6.88-6.79 (m, |
| | dimethyl-4-oxo-1H-pyridine-3- | | 1H), 3.84 (d, J = 1.7 Hz, 3H), 2.54 (s, |
| | carbonitrile | | 3H), 1.87 (s, 3H). |
| 83 | ethyl 2-[2-(3,4-difluoro-2-methyl- | 478.49; | ¹H NMR (400 MHz, CD₃OD) δ 8.21- |
| | phenoxy)-4-methyl-3-quinolyl]- | 479.3 | 8.13 (m, 1H), 7.80-7.68 (m, 2H), 7.66- |
| | 5,6-dimethyl-4-oxo-1H-pyridine- | | 7.56 (m, 1H), 7.16 (q, J = 9.2 Hz, 1H), |
| | 3-carboxylate | | 6.95-6.86 (m, 1H), 4.23-4.01 (m, 2H), |
| | | | 2.70 (s, 3H), 2.59 (s, 3H), 2.36 (s, 3H), |
| | | | 2.06 (d, J = 2.2 Hz, 3H), 0.60 (t, J = 7.1 |
| | | | Hz, 3H). |
| 84 | 2-[2-(3,4-difluoro-2-methoxy- | 459.34; | ¹H NMR (400 MHz, CD₃OD) δ 7.58 (d, |
| | phenoxy)-5-fluoro-4- | 460.4 | J = 10.2 Hz, 1H), 7.17 (d, J = 5.8 Hz, |
| | (trifluoromethyl)phenyl]-3- | | 1H), 7.08-6.94 (m, 1H), 6.91-6.79 (m, |
| | methoxy-6-methyl-1H-pyridin-4- | | 1H), 6.39 (s, 1H), 3.83 (d, J = 1.4 Hz, |
| | one | | 3H), 3.72 (s, 3H), 2.34 (s, 3H). |
| 85 | 2-[2-(4,4-difluoroazepan-1-yl)-3- | 399.43; | ¹H NMR (400 MHz, CD₃OD) δ 8.65 (s, |
| | quinolyl]-3-methoxy-6-methyl- | 400.3 | 1H), 8.05 (d, J = 8.6 Hz, 1H), 8.01-7.96 |
| | 1H-pyridin-4-one | | (m, 1H), 7.96-7.89 (m, 1H), 7.69-7.55 |
| | | | (m, 1H), 7.21 (s, 1H), 3.84 (s, 3H), 3.80- |
| | | | 3.67 (m, 2H), 3.53 (t, J = 5.7 Hz, 2H), |
| | | | 2.69 (s, 3H), 2.44-2.25 (m, 2H), 2.24- |
| | | | 2.02 (m, 2H), 2.02-1.81 (m, 2H). |
| 86 | ethyl 2-[2-(4,4-difluoroazepan-1- | 441.47; | ¹H NMR (400 MHz, CD₃OD) δ 8.24 (s, |
| | yl)-3-quinolyl]-6-methyl-4-oxo- | 442.6 | 1H), 7.90-7.80 (m, 2H), 7.76 (app t, J = |
| | 1H-pyridine-3-carboxylate | | 7.5 Hz, 1H), 7.45 (app t, J = 7.5 Hz, 1H), |
| | | | 6.82 (s, 1H), 4.11 (q, J = 7.1 Hz, 2H), |
| | | | 3.72-3.65 (m, 2H), 3.58-3.42 (m, 2H), |
| | | | 2.54 (s, 3H), 2.38-2.23 (m, 2H), 2.16- |
| | | | 2.02 (m, 2H), 1.93-1.83 (m, 2H), 0.92 |
| | | | (t, J = 7.1 Hz, 3H). |
| 87 | ethyl 2-[2-[(3R,5S)-4,4-difluoro- | 501.49; | ¹H NMR (400 MHz, CD₃OD) δ 7.61 (s, 1H), |
| | 3,5-dimethyl-1-piperidyl]-5- | 502.4 | 4.07 (q, J = 7.1 Hz, 2H), 3.58 (d, J = |
| | methyl-6-(trifluoromethyl)-3- | | 13.2 Hz, 2H), 2.70 (t, J = 12.6 Hz, 2H), |
| | pyridyl]-5,6-dimethyl-4-oxo-1H- | | 2.42 (s, 3H), 2.40 (q, J = 2.1 Hz, 3H), |
| | pyridine-3-carboxylate | | 2.11 (s, 3H), 2.08-1.95 (m, 2H), 0.97 (t, |
| | | | J = 7.1 Hz, 3H), 0.91 (d, J = 6.8 Hz, 6H). |
| 88 | ethyl 2-[2-(4,4-difluoro-1- | 473.44; | ¹H NMR (400 MHz, CD₃OD) δ 7.68 (s, |
| | piperidyl)-5-methyl-6- | 474.2 | 1H), 4.07 (q, J = 7.2 Hz, 2H), 3.33-3.29 |
| | (trifluoromethyl)-3-pyridyl]-5,6- | | (m, 4H, obscured by solvent peak), 2.47 |
| | dimethyl-4-oxo-1H-pyridine-3- | | (s, 3H), 2.42 (q, J = 2.2 Hz, 3H), 2.14 (s, |
| | carboxylate | | 3H), 2.00-1.86 (m, 4H), 0.94 (t, J = 7.1 |
| | | | Hz, 3H). |
| 89 | ethyl 6-[2-(3,4-difluoro-2- | 515.4; | ¹H NMR (400 MHz, DMSO-d₆) δ 11.57 |
| | methoxy-phenoxy)-5-fluoro-4- | 516.5 | (s, 1H), 7.84 (d, J = 10.4 Hz, 1H), 7.28 |
| | (trifluoromethyl)phenyl]-2,5- | | (d, J = 5.9 Hz, 1H), 7.23-7.11 (m, 1H), |
| | dimethyl-4-oxo-1H-pyridine-3- | | 6.94-6.85 (m, 1H), 4.20 (q, J = 7.1 Hz, |
| | carboxylate | | 2H), 3.79 (d, J = 1.3 Hz, 3H), 2.19 (s, |
| | | | 3H), 1.71 (s, 3H), 1.24 (t, J = 7.1 Hz, |
| | | | 3H). |
| 90 | ethyl 2-[2-(3,4-difluoro-2-methyl- | 496.43; | ¹H NMR (400 MHz, DMSO-d₆) δ 11.71 |
| | phenoxy)-4-methyl-5- | 497.3 | (s, 1H), 8.55 (s, 1H), 7.36 (q, J = 9.4 Hz, |
| | (trifluoromethyl)-3-pyridyl]-5,6- | | 1H), 6.88 (ddd, J = 9.1, 4.3, 1.9 Hz, 1H), |
| | dimethyl-4-oxo-1H-pyridine-3- | | 3.97 (qd, J = 7.1, 3.0 Hz, 2H), 2.30 (d, |
| | carboxylate | | J = 5.7 Hz, 6H), 2.02 (d, J = 2.1 Hz, 3H), |
| | | | 1.92 (s, 3H), 0.82 (t, J = 7.1 Hz, 3H). |
| 91 | 2-[2-(3,4-difluoro-2-methyl- | 378.37; | ¹H NMR (400 MHz, CD₃OD) δ 8.61 (s, 1H), |
| | phenoxy)-3-quinolyl]-3-methyl- | 379.3 | 8.53 (d, J = 6.7 Hz, 1H), 8.02 (dd, J = |
| | 1H-pyridin-4-one | | 8.1, 1.4 Hz, 1H), 7.83-7.73 (m, 2H), |
| | | | 7.60 (ddd, J = 8.1, 6.6, 1.5 Hz, 1H), 7.34 |
| | | | (d, J = 6.8 Hz, 1H), 7.19 (q, J = 9.3 Hz, |
| | | | 1H), 7.01 (ddd, J = 9.2, 4.2, 2.1 Hz, 1H), |
| | | | 2.27 (s, 3H), 2.04 (d, J = 2.2 Hz, 3H). |
| 92 | ethyl 2-[2-(3,4-difluoro-2- | 515.4; | ¹H NMR (400 MHz, DMSO-d₆) δ 11.67 |
| | methoxy-phenoxy)-5-fluoro-4- | 516.5 | (s, 1H), 7.69-7.62 (m, 1H), 7.25-7.13 |
| | (trifluoromethyl)phenyl]-5,6- | | (m, 2H), 6.87-6.82 (m, 1H), 3.97 (q, J = |
| | dimethyl-4-oxo-1H-pyridine-3- | | 7.1 Hz, 2H), 3.80 (d, J = 1.0 Hz, 3H), |
| | carboxylate | | 2.24 (s, 3H), 1.89 (s, 3H), 0.88 (t, J = 7.1 |
| | | | Hz, 3H). |
| 93 | 2-[2-(4,4-difluoroazepan-1-yl)-5- | 475.45; | ¹H NMR (400 MHz, CD₃OD) δ 7.69 (s, |
| | methyl-6-(trifluoromethyl)-3- | 476.3 | 1H), 6.75 (s, 1H), 4.15-4.01 (m, 2H), |
| | pyridyl]-3-(2-methoxyethoxy)-6- | | 3.63-3.52 (m, 2H), 3.42-3.36 (m, 2H), |
| | methyl-1H-pyridin-4-one | | 3.16-3.12 (m, 2H), 3.12 (s, 3H), 2.49 (s, |
| | | | 3H), 2.42-2.35 (m, 3H), 2.32-2.18 (m, |
| | | | 2H), 2.03-1.91 (m, 2H), 1.84-1.75 (m, |
| | | | 2H). |
| 94 | 2-[2-(3,4-difluoro-2-methyl- | 452.45; | ¹H NMR (400 MHz, CD₃OD) δ 8.62 (s, |
| | phenoxy)-3-quinolyl]-3-(2- | 453.3 | 1H), 8.04-7.96 (m, 1H), 7.84-7.66 (m, |
| | methoxyethoxy)-6-methyl-1H- | | 2H), 7.64-7.53 (m, 1H), 7.25 (s, 1H), |
| | pyridin-4-one | | 7.22-7.12 (m, 1H), 7.10-7.01 (m, 1H), |
| | | | 4.22-4.12 (m, 2H), 3.43-3.36 (m, 2H), |
| | | | 2.90 (s, 3H), 2.69 (s, 3H), 2.10 (d, J = 2.2 |
| | | | Hz, 3H). |
| 95 | 2-[5-chloro-2-(3,4-difluoro-2- | 461.77; | ¹H NMR (500 MHz, Acetonitrile-d3) δ |
| | methoxy-phenoxy)-4- | 462.0 | 8.23-8.20 (m, 2H), 7.19 (d, J = 2.0 Hz, |
| | (trifluoromethyl)phenyl]-6- | | 2H), 7.01 (ddd, J = 10.0, 9.3, 8.3 Hz, |
| | methoxy-1H-pyridin-4-one | | 1H), 6.88 (ddd, J = 9.4, 5.0, 2.4 Hz, 1H), |
| | | | 6.20 (d, J = 1.8 Hz, 1H), 3.90 (s, 3H), |
| | | | 3.83 (d, J = 1.4 Hz, 3H). |
| 96 | ethyl 2-[6-(3,3- | 512.51; | |
| | difluorocyclobutyl)-4-[4- | 513.6 | |
| | (trifluoromethyl)cyclohexyl]-3- | | |
| | pyridyl]-5,6-dimethyl-4-oxo-1H- | | |
| | pyridine-3-carboxylate | | |
| 97 | isopropyl 2-[2-(4,4- | 501.49; | ¹H NMR (400 MHz, CD₃OD) δ 7.51 (s, |
| | difluoroazepan-1-yl)-5-methyl-6- | 502.6 | 1H), 4.99 (hept, J = 6.2 Hz, 1H), |
| | (trifluoromethyl)-3-pyridyl]-5,6- | | 3.59-3.52 (m, 2H), 3.35-3.32 (m, 2H) |
| | dimethyl-4-oxo-1H-pyridine-3- | | overlaps with CD₂HOD, 2.42 (s, 3H), |
| | carboxylate | | 2.35 (q, J = 2.2 Hz, 3H), 2.32-2.18 (m, |
| | | | 2H), 2.11 (s, 3H), 2.06-1.93 (m, 2H), |
| | | | 1.87-1.77 (m, 2H), 1.01 (d, J = 6.2 Hz, |
| | | | 6H). |
| 98 | 6-[2-(4,4-difluoroazepan-1-yl)-5- | 431.4; | ¹H NMR (400 MHz, DMSO-d₆) δ 7.70 |
| | methyl-6-(trifluoromethyl)-3- | 432.3 | (s, 1H), 6.63 (s, 1H), 3.77 (s, 3H), 3.48- |
| | pyridyl]-3-methoxy-2-methyl-1H- | | 3.43 (m, 2H), 3.19 (t, J = 6.1 Hz, 2H), |
| | pyridin-4-one | | 2.40-2.28 (m, 6H), 2.29-2.12 (m, 2H), |
| | | | 1.96 (dd, J = 15.9, 10.1 Hz, 2H), 1.71 (dt, |
| | | | J = 11.9, 6.6 Hz, 2H). |
| 99 | 6-[2-(3,4-difluoro-2-methyl- | 408.4; | ¹H NMR (400 MHz, DMSO-d₆) δ 8.77 |
| | phenoxy)-3-quinolyl]-3-methoxy- | 409.2 | (s, 1H), 8.08 (d, J = 8.0 Hz, 1H), 7.75 (t, |
| | 2-methyl-1H-pyridin-4-one | | J = 7.7 Hz, 1H), 7.66 (d, J = 8.3 Hz, 1H), |
| | | | 7.58 (t, J = 7.5 Hz, 1H), 7.40 (dd, J = |
| | | | 19.3, 9.8 Hz, 2H), 7.21 (ddd, J = 9.0, 4.4, |
| | | | 1.9 Hz, 1H), 3.87 (s, 3H), 2.54 (s, 3H), |
| | | | 2.08 (d, J = 2.0 Hz, 3H). |
| 100 | 2-[2-(3,4-difluoro-2-methyl- | 407.41; | ¹H NMR (400 MHz, DMSO-d₆) δ 12.23 |
| | phenoxy)-3-quinolyl]-6- | 408.4 | (s, 1H), 8.77 (s, 1H), 8.06 (dd, J = 8.0, |
| | (dimethylamino)-1H-pyridin-4-one | | 1.5 Hz, 1H), 7.72 (s, 1H), 7.64 (d, J = 8.2 |
| | | | Hz, 1H), 7.57 (d, J = 7.8 Hz, 1H), 7.39 |
| | | | (q, J = 9.4 Hz, 1H), 7.17 (s, 1H), 6.87 (s, |
| | | | 1H), 6.27 (s,1H), 3.15 (s, 6H), 2.07 (d, |
| | | | J = 2.1 Hz, 3H). |
| 101 | isopropyl 2-[2-(3,4-difluoro-2- | 478.49; | ¹H NMR (400 MHz, CD₃OD) δ 8.51 (s, |
| | methyl-phenoxy)-3-quinolyl]-5,6- | 479.5 | 1H), 8.02-7.96 (m, 1H), 7.84-7.73 (m, |
| | dimethyl-4-oxo-1H-pyridine-3- | | 2H), 7.60 (ddd, J = 8.2, 6.5, 1.8 Hz, 1H), |
| | carboxylate | | 7.19 (app q, J = 9.3 Hz, 1H), 7.00 (app |
| | | | ddd, J = 9.1, 4.1, 2.0 Hz, 1H), 5.08 (hept, |
| | | | J = 6.1 Hz, 1H), 2.71 (s, 3H), 2.36 (s, |
| | | | 3H), 2.06 (d, J = 2.2 Hz, 3H), 0.79 (d, |
| | | | J = 6.2 Hz, 6H). |
| 102 | 2-[2-tert-butyl-4-(4,4- | 361.43; | ¹H NMR (400 MHz, DMSO-d₆) δ 14.28 |
| | difluorocyclohexyl)pyrimidin-5- | 362.3 | (s, 1H), 8.78 (s, 1H), 7.19 (s, 1H), 7.11 |
| | yl]-6-methyl-1H-pyridin-4-one | | (s, 1H), 2.88 (s, 1H), 2.57 (s, 3H), 2.09 |
| | | | (d, J = 9.2 Hz, 2H), 1.87 (d, J = 9.1 Hz, |
| | | | 6H), 1.40 (s, 9H). |
| 103 | 2-[5-chloro-2-(3,4-difluoro-2- | 431.74; | ¹H NMR (500 MHz, DMSO-d₆) δ 10.92 |
| | methoxy-phenoxy)-4- | 432.0 | (s, 1H), 8.38 (s, 1H), 8.13 (s, 1H), 7.38 |
| | (trifluoromethyl)phenyl]-1H- | | (s, 1H), 7.27-7.18 (m, 2H), 7.05 (ddd, J = |
| | pyridin-4-one | | 9.5, 5.1, 2.2 Hz, 1H), 6.79 (s, 1H), 3.81 |
| | | | (d, J = 1.2 Hz, 3H). |
| 104 | 2-[2-(4,4-difluoroazepan-1-yl)-5- | 459.45; | ¹H NMR (400 MHz, CD₃OD) δ 7.63 (s, |
| | methyl-6-(trifluoromethyl)-3- | 460.3 | 1H), 6.36 (s, 1H), 4.60-4.37 (m, 1H), |
| | pyridyl]-3-isopropoxy-6-methyl- | | 3.64-3.51 (m, 2H), 3.16 (t, J = 6.2 Hz, |
| | 1H-pyridin-4-one | | 2H), 2.40-2.35 (m, 6H), 2.34-2.14 (m, |
| | | | 2H), 2.02-1.89 (m, 2H), 1.86-1.70 (m, |
| | | | 2H), 0.91 (d, J = 6.2 Hz, 6H). |
| 105 | 2-(2-benzyloxy-3-quinolyl)-6- | 342.39; | |
| | methyl-1H-pyridin-4-one | 343.5 | |
| 106 | 2-[2-(3,4-difluoro-2-methyl- | 436.45; | ¹H NMR (400 MHz, CD₃OD) δ 8.48 (s, |
| | phenoxy)-3-quinolyl]-3- | 437.3 | 1H), 8.00-7.93 (m, 1H), 7.78-7.65 (m, |
| | isopropoxy-6-methyl-1H-pyridin- | | 2H), 7.60-7.47 (m, 1H), 7.22-7.08 (m, |
| | 4-one | | 1H), 7.05-6.95 (m, 1H), 6.57 (s, 1H), |
| | | | 4.70-4.55 (m, 1H), 2.44 (s, 3H), 2.12 (d, |
| | | | J = 2.2 Hz, 3H), 1.02 (d, J = 6.0 Hz, 6H). |
| 107 | 2-[2-(3,4-difluoro-2-methyl- | 408.4; | ¹H NMR (400 MHz, CD₃OD) δ 8.47 (s, |
| | phenoxy)-3-quinolyl]-3-methoxy- | 409.25 | 1H), 7.98-7.91 (m, 1H), 7.76-7.65 (m, |
| | 6-methyl-1H-pyridin-4-one | | 2H), 7.57-7.47 (m, 1H), 7.15 (q, J = 9.3 |
| | | | Hz, 1H), 7.05-6.94 (m, 1H), 6.46 (s, |
| | | | 1H), 3.71 (s, 3H), 2.39 (s, 3H), 2.09 (d, |
| | | | J = 2.2 Hz, 3H). |
| 108 | 2-[2-(4,4-difluoroazepan-1-yl)-5- | 431.4; | ¹H NMR (400 MHz, CD₃OD) δ 7.63 (s, |
| | methyl-6-(trifluoromethyl)-3- | 432.23 | 1H), 6.50 (s, 1H), 3.70 (s, 3H), 3.57- |
| | pyridyl]-3-methoxy-6-methyl-1H- | | 3.52 (m, 2H), 3.23 (t, J = 6.2 Hz, 2H), |
| | pyridin-4-one | | 2.41-2.35 (m, 6H), 2.29-2.14 (m, 2H), |
| | | | 2.04-1.91 (m, 2H), 1.85-1.73 (m, 2H). |
| 109 | 2-[4-(4,4-difluorocyclohexyl)-2- | 373.32; | ¹H NMR (400 MHz, CD₃OD) δ 8.97 (s, |
| | (trifluoromethyl)pyrimidin-5-yl]- | 374.2 | 1H), 6.96 (s, 1H), 6.92 (s, 1H), 3.00 (t, |
| | 6-methyl-1H-pyridin-4-one | | J = 11.6 Hz, 1H), 2.60 (s, 3H), 2.20-2.00 |
| | | | (m, 4H), 1.95-1.72 (m, 4H). |
| 110 | 2-[2-(3,4-difluoro-2-mcthyl- | 436.45; | ¹H NMR (400 MHz, CD₃OD) δ 8.56 (s, |
| | phenoxy)-3-quinolyl]-3- | 437.3 | 1H), 8.01-7.94 (m, 1H), 7.81-7.71 (m, |
| | (ethoxymethyl)-6-methyl-1H- | | 2H), 7.62-7.55 (m, 1H), 7.24-7.10 (m, |
| | pyridin-4-one | | 1H), 7.08-6.99 (m, 1H), 6.93 (s, 1H), |
| | | | 4.45 (s, 2H), 3.36 (q, J = 7.0 Hz, 2H), |
| | | | 2.60 (s, 3H), 2.07 (d, J = 2.2 Hz, 3H), |
| | | | 0.93 (t, J = 7.0 Hz, 3H). |
| 111 | 2-[2-(4,4-difluoroazepan-1-yl)-5- | 415.4; | ¹H NMR (400 MHz, CD₃OD) δ 7.62 (s, |
| | methyl-6-(trifluoromethyl)-3- | 416.3 | 1H), 6.64 (s, 1H), 3.69-3.41 (m, 2H), |
| | pyridyl]-3,6-dimethyl-1H-pyridin- | | 3.22-3.04 (m, 2H), 2.47 (s, 3H), 2.40 (q, |
| | 4-one | | J = 2.1 Hz, 3H), 2.36-2.12 (m, 2H), 2.04- |
| | | | 1.92 (m, 2H), 1.91 (s, 3H), 1.85-1.71 |
| | | | (m, 2H). |
| 112 | 2-[2-(3,4-difluoro-2-methyl- | 392.4; | ¹H NMR (400 MHz, CD₃OD) δ 8.50 (s, |
| | phenoxy)-3-quinolyl]-3,6- | 393.2 | 1H), 8.02-7.97 (m, 1H), 7.80-7.71 (m, |
| | dimethyl-1H-pyridin-4-one | | 2H), 7.61-7.53 (m, 1H), 7.23-7.12 (m, |
| | | | 1H), 7.03-6.95 (m, 1H), 6.75 (s, 1H), |
| | | | 2.53 (s, 3H), 2.09 (s, 3H), 2.06 (d, J = 2.2 |
| | | | Hz, 3H). |
| 113 | 2-[2-(3,4-difluoro-2-methoxy- | 429.31; | ¹H NMR (400 MHz, DMSO-d₆) δ 10.67 |
| | phenoxy)-5-fluoro-4- | 430.4 | (s, 1H), 7.93 (d, J = 11.4 Hz, 1H), 7.26- |
| | (trifluoromethyl)phenyl]-6-methyl- | | 7.12 (m, 3H), 6.96-6.88 (m, 1H), 6.66 |
| | 1H-pyridin-4-one | | (s, 1H), 3.82 (d, J = 1.2 Hz, 3H), 2.40 (s, |
| | | | 3H). |
| 114 | 2-[5-chloro-2-(3,4-difluoro-2- | 445.77; | ¹H NMR (500 MHz, DMSO-d₆) δ 10.74 |
| | methoxy-phenoxy)-4- | 446.4 | (s, 1H), 8.12 (s, 1H), 7.26-7.17 (m, 3H), |
| | (trifluoromethyl)phenyl]-6-methyl- | | 7.02 (ddd, J = 9.3, 5.0, 2.1 Hz, 1H), 6.67 |
| | 1H-pyridin-4-one | | (s, 1H), 3.82 (d, J = 1.1 Hz, 3H), 2.41 (s, |
| | | | 3H). |
| 115 | 2-[2-(3,4-difluoro-2-methyl- | 364.34; | ¹H NMR (400 MHz, DMSO-d₆) δ 8.86 |
| | phenoxy)-3-quinolyl]-1H-pyridin- | 365.3 | (s, 1H), 8.68 (d, J = 6.8 Hz, 1H), 8.08 |
| | 4-one | | (dd, J = 8.2, 1.5 Hz, 1H), 7.79 (ddd, J = |
| | | | 8.5, 6.9, 1.5 Hz, 1H), 7.71 (d, J = 2.5 Hz, |
| | | | 1H), 7.68 (d, J = 8.4 Hz, 1H), 7.61 (ddd, |
| | | | J = 8.1, 6.9, 1.2 Hz, 1H), 7.41 (q, J = |
| | | | 9.4 Hz, 1H), 7.34-7.26 (m, 2H), 2.06 (d, |
| | | | J = 2.0 Hz, 3H). |
| 116 | ethyl 2-[2-(3,4-difluoro-2-methyl- | 464.46; | ¹H NMR (400 MHz, CD₃OD) δ 8.54 (s, |
| | phenoxy)-3-quinolyl]-5,6- | 465.6 | 1H), 8.00 (app d, J = 8.1 Hz, 1H), 7.84- |
| | dimethyl-4-oxo-1H-pyridine-3- | | 7.72 (m, 2H), 7.60 (app ddd, J = 8.1, 6.6, |
| | carboxylate | | 1.5 Hz, 1H), 7.19 (app q, J = 9.2 Hz, 1H), |
| | | | 6.98-6.90 (m, 1H), 4.19 (q, J = 7.1 Hz, |
| | | | 2H), 2.76 (s, 3H), 2.40 (s, 3H), 2.05 (d, |
| | | | J = 2.2 Hz, 3H), 0.64 (t, J = 7.1 Hz, 3H). |
| 117 | ethyl 2-[2-(4,4-difluoroazepan-1- | 487.46; | ¹H NMR (400 MHz, CD₃OD) δ 7.54 (s, |
| | yl)-5-methyl-6-(trifluoromethyl)- | 488.7 | 1H), 4.10 (q, J = 7.1 Hz, 2H), 3.58-3.51 |
| | 3-pyridyl]-5,6-dimethyl-4-oxo-1H- | | (m, 2H), 3.27 (m, 2H) overlaps with |
| | pyridine-3-carboxylate | | CD₂HOD, 2.44 (s, 3H), 2.36 (q, J = 2.2 |
| | | | Hz, 3H), 2.32-2.17 (m, 2H), 2.12 (s, |
| | | | 3H), 2.06-1.91 (m, 2H), 1.86-1.76 (m, |
| | | | 2H), 0.99 (t, J = 7.1 Hz, 3H). |
| 118 | ethyl 2-[2-(4,4-difluoroazepan-1- | 473.44; | ¹H NMR (400 MHz, CD₃OD) δ 7.61 (s, |
| | yl)-5-methyl-6-(trifluoromethyl)- | 474.5 | 1H), 6.75 (s, 1H), 4.13 (q, J = 7.1 Hz, |
| | 3-pyridyl ]-6-methyl-4-oxo-1H- | | 2H), 3.58-3.51 (m, 2H), 3.25 (br s, 2H) |
| | pyridine-3-carboxylate | | overlaps with CD₂HOD, 2.51 (s, 3H), |
| | | | 2.38 (q, J = 2.2 Hz, 3H), 2.34-2.20 (m, |
| | | | 2H), 2.06-1.92 (m, 2H), 1.88-1.78 (m, |
| | | | 2H), 1.02 (t, J = 7.1 Hz, 3H). |
| 119 | 6-[2-(3,4-difluoro-2-methyl- | 408.4; | ¹H NMR (400 MHz, CD₃OD) δ 8.53 (s, |
| | phenoxy)-3-quinolyl]-3- | 409.5 | 1H), 7.98 (app d, J = 8.2 Hz, 1H), 7.76- |
| | (hydroxymethyl)-2-methyl-1H- | | 7.65 (m, 2H), 7.54 (app ddd, J = 8.2, 6.3, |
| | pyridin-4-one | | 1.9 Hz, 1H), 7.18 (app q, J = 9.2 Hz, 1H), |
| | | | 7.06 (app ddd, J = 9.1, 4.2, 2.0 Hz, 1H), |
| | | | 6.74 (br s, 1H), 4.68 (s, 2H), 2.55 (s, 3H), |
| | | | 2.07 (d, J = 2.2 Hz, 3H). |
| 120 | ethyl 6-[2-(4,4-difluoroazepan-1- | 473.44; | ¹H NMR (400 MHz, CD₃OD) δ 7.67 (s, |
| | yl)-5-methyl-6-(trifluoromethyl)- | 474.6 | 1H), 6.55 (s, 1H), 4.38 (q, J = 7.2 Hz, |
| | 3-pyridyl]-2-methyl-4-oxo-1H- | | 2H), 3.62-3.54 (m, 2H), 3.29-3.24 (m, |
| | pyridine-3-carboxylate | | 2H) overlaps with CD₂HOD, 2.45 (s, |
| | | | 3H), 2.39 (app q, J = 2.1 Hz, 3H), 2.36- |
| | | | 2.21 (m, 2H), 2.05-1.91 (m, 2H), 1.88 - |
| | | | 1.78 (m, 2H), 1.37 (t, J = 7.1 Hz, 3H). |
| 121 | ethyl 2-[2-(3,4-difluoro-2-methyl- | 450.43; | ¹H NMR (400 MHz, CD₃OD) δ 8.49 (s, |
| | phenoxy)-3-quinolyl]-6-methyl-4- | 451.6 | 1H), 8.01-7.94 (m, 1H), 7.82-7.69 (m, |
| | oxo-1H-pyridine-3-carboxylate | | 2H), 7.57 (app ddd, J = 8.1, 6.6, 1.6 Hz, |
| | | | 1H), 7.19 (app q, J = 9.3 Hz, 1H), 7.02 |
| | | | (app ddd, J = 9.1, 4.3, 2.1 Hz, 1H), 6.93 |
| | | | (s, 1H), 4.09 (q, J = 7.1 Hz, 2H), 2.60 (s, |
| | | | 3H), 2.08 (d, J = 2.2 Hz, 3H), 0.80 (t, J = |
| | | | 7.1 Hz, 3H). |
| 122 | 2-[2-(3,4-difluoro-2-methyl- | 408.4; | ¹H NMR (400 MHz, CD₃OD) δ 8.48 (s, |
| | phenoxy)-3-quinolyl]-3- | 409.5 | 1H), 7.96 (app d, J = 8.1 Hz, 1H), 7.77- |
| | (hydroxymethyl)-6-methyl-1H- | | 7.68 (m, 2H), 7.58-7.50 (m, 1H), 7.15 |
| | pyridin-4-one | | (app q, J = 9.3 Hz, 1H), 7.02 (app ddd, |
| | | | J = 9.1, 4.2, 2.0 Hz, 1H), 6.40 (s, 1H), 4.43 |
| | | | (br s, 2H), 2.39 (s, 3H), 2.07 (d, J = 2.2 |
| | | | Hz, 3H). |
| 123 | ethyl 6-[2-(3,4-difluoro-2-methyl- | 450.43; | ¹H NMR (400 MHz, CD₃OD) δ 8.60 (s, |
| | phenoxy)-3-quinolyl]-2-methyl-4- | 451.4 | 1H), 8.03-7.96 (m, 1H), 7.78-7.66 (m, |
| | oxo-1H-pyridine-3-carboxylate | | 2H), 7.55 (app ddd, J = 8.1, 6.5, 1.6 Hz, |
| | | | 1H), 7.18 (app q, J = 9.2 Hz, 1H), 7.07 |
| | | | (app ddd, J = 9.0, 4.3, 2.0 Hz, 1H), 6.91 |
| | | | (br s, 1H), 4.40 (q, J = 7.1 Hz, 2H), 2.53 |
| | | | (s, 3H), 2.09 (d, J = 2.2 Hz, 3H), 1.39 (t, |
| | | | J = 7.1 Hz, 3H). |
| 124 | 2-12-(4,4-difluoroazepan-1-yl)-5- | 431.4; | ¹H NMR (400 MHz, CD₃OD) δ 7.71 (s, |
| | methyl-6-(trifluoromethyl)-3- | 432.6 | 1H), 6.93 (s, 1H), 4.52 (br s, 2H), 3.56 |
| | pyridyl]-3-(hydroxymethyl)-6- | | (br s, 2H), 3.14 (br s, 2H), 2.57 (s, 3H), |
| | methyl-1H-pyridin-4-one | | 2.40 (app q, J = 2.1 Hz, 3H), 2.31-2.17 |
| | | | (m, 2H), 2.02-1.89 (m, 2H), 1.83-1.72 |
| | | | (m, 2H). |
| 125 | 6-[2-(4,4-difluoroazepan-1-yl)-5- | 431.4; | ¹H NMR (400 MHz, CD₃OD) δ 7.78 (s, |
| | methyl-6-(trifluoromethyl)-3- | 432.5 | 1H), 7.13 (s, 1H), 4.78 (s, 2H), 3.61- |
| | pyridyl]-3-(hydroxymethyl)-2- | | 3.54 (m, 2H), 3.14 (t, J = 6.0 Hz, 2H), |
| | methyl-1H-pyridin-4-one | | 2.75 (s, 3H), 2.42 (app q, J = 2.1 Hz, 3H), |
| | | | 2.38-2.24 (m, 2H), 2.05-1.90 (m, 2H), |
| | | | 1.87-1.76 (m, 2H). |
| 126 | 2-[5-[2-methoxy-4- | 460.33; | ¹H NMR (500 MHz, DMSO-d₆) δ 10.73 |
| | (trifluoromethoxy)phenoxy]-2- | 461.5 | (s, 1H), 8.33 (s, 1H), 8.16 (s, 1H), 7.46 |
| | (trifluoromethyl)-4-pyridyl]-6- | | (s, 1H), 7.38 (d, J = 8.8 Hz, 1H), 7.27 (d, |
| | methyl-1H-pyridin-4-one | | J = 2.8 Hz, 1H), 7.03 (ddd, J = 8.8, 2.8, |
| | | | 1.3 Hz, 1H), 6.72 (s, 1H), 3.80 (s, 3H), |
| | | | 2.46 (s, 3H). |
| 127 | 2-[5-(3,4-difluoro-2-methoxy- | 412.31; | ¹H NMR (500 MHz, DMSO-d₆) δ 10.85 |
| | phenoxy)-2-(trifluoromethyl)-4- | 413.4 | (s, 1H), 8.33 (s, 2H), 7.38 (s, 1H), 7.29- |
| | pyridyl]-6-methyl-1H-pyridin-4- | | 7.20 (m, 1H), 7.17-7.11 (m, 1H), 6.71 |
| | one | | (s, 1H), 3.84 (s, 3H), 2.44 (s, 3H). |
| 128 | 6-[2-(7,7-difluoro-3,3a,4,5,6,7a- | 441.44; | ¹H NMR (400 MHz, CD₃OD) δ 7.66 (s, |
| | hexahydro-1H-isoindol-2-yl)-5- | 442.3 | 1H), 6.77 (s, 1H), 3.44 (m, 1H), 3.42 (m, |
| | methyl-6-(trifluoromethyl)-3- | | 1H), 3.03 (m, 1H), 2.68 (m, 1H), 2.56 (s, |
| | pyridyl]-2,3-dimethyl-1H-pyridin- | | 3H), 2.46 (s, 1H), 2.38 (q, J = 2.2 Hz, |
| | 4-one | | 3H), 2.21 (s, 3H), 1.94-1.81 (m, 1H), |
| | | | 1.73 (m, 1H), 1.60 (m, 3H), 1.32 (m, 8.4 |
| | | | Hz, 2H). |
| 129 | 6-[2-(3,4-difluoro-2-methyl- | 410.34; | |
| | phenoxy)-5-(trifluoromethyl)-3- | 411.1 | |
| | pyridyl]-2,3-dimethyl-1H-pyridin- | | |
| | 4-one | | |
| 130 | 2-[2-(3,4-difluoro-2-methyl- | 396.31; | ¹HNMR (400 MHz, CD₃OD) δ 8.63 (dd, |
| | phenoxy)-5-(trifluoromethyl)-3- | 397.1 | J = 2.4, 1.0 Hz, 1H), 8.55 (d, J = 2.4 Hz, |
| | pyridyl]-6-methyl-1H-pyridin-4- | | 1H), 7.45 (d, J = 2.4 Hz, 1H), 7.19 (q, |
| | one | | J = 9.3 Hz, 1H), 7.13 (d, J = 2.4 Hz, 1H), |
| | | | 7.02 (ddd, J = 9.1, 4.1, 2.1 Hz, 1H), 2.71 |
| | | | (s, 3H), 2.07 (d, J = 2.2 Hz, 3H). |
| 131 | 2,3-dimethyl-6-[6- | 418.38; | ¹H NMR (400 MHz, CD₃OD) δ 8.64 (s, |
| | (trifluoromethyl)-4-[4- | 419.23 | 1H), 7.73 (s, 1H), 6.39 (s, 1H), 2.99- |
| | (trifluoromethyl)cyclohexyl]-3- | | 2.73 (m, 1H), 2.51-2.33 (m, 4H), 2.10 |
| | pyridyl]-1H-pyridin-4-one | | (s, 4H), 2.05 (s, 1H), 1.85-1.62 (m, 6H). |
| 132 | 6-[4-tert-butyl-2-(4-fluoro-2- | 425.49; | ¹H NMR (400 MHz, DMSO-d₆) δ 11.29 |
| | methoxy-phenoxy)-6-methyl- | 426.3 | (br s, 1H), 7.10-7.00 (m, 3H), 6.77 (td, |
| | phenyl]-3-(hydroxymethyl)-2- | | J = 8.5, 2.8 Hz, 1H), 6.46 (s, 1H), 5.97 |
| | methyl-1H-pyridin-4-one | | (s, 1H), 4.88 (br s, 1H), 4.37 (s, 2H), 3.73 |
| | | | (s, 3H), 2.29 (s, 3H), 2.19 (s, 3H), 1.17 |
| | | | (s, 9H). |
| 133 | 6-[2-(4,4-difluoroazepan-1-yl)-5- | 415.4; | ¹H NMR (400 MHz, CD₃OD) δ 7.77 (s, |
| | methyl-6-(trifluoromethyl)-3- | 416.2 | 1H), 7.10 (s, 1H), 3.60-3.53 (m, 2H), |
| | pyridyl]-2,3-dimethyl-1H-pyridin- | | 3.13 (t, J = 6.0 Hz, 2H), 2.66 (s, 3H), |
| | 4-one | | 2.41 (s, 3H), 2.35-2.22 (m, 5H), 2.04- |
| | | | 1.92 (m, 2H), 1.81 (m, 2H). |
| 134 | 2-[2-(4,4-difluoroazepan-1-yl)-3- | 369.41; | ¹H NMR (400 MHz, DMSO-d₆) δ 14.50 |
| | quinolyl]-6-methyl-1H-pyridin-4- | 370.3 | (s, 1H), 8.41 (s, 1H), 7.91-7.84 (m, 1H), |
| | one | | 7.78-7.69 (m, 2H), 7.41 (ddd, J = 8.1, |
| | | | 6.3, 1.8 Hz, 1H), 7.36 (d, J = 2.4 Hz, 1H), |
| | | | 7.19 (d, J = 2.1 Hz, 1H), 3.63-3.51 (m, |
| | | | 2H), 3.24 (t, J = 6.0 Hz, 2H), 2.63 (s, |
| | | | 3H), 2.33 (t, J = 1.8 Hz, 2H), 2.11-1.94 |
| | | | (m, 2H), 1.83-1.64 (m, 2H). |
| 135 | 6-[4-tert-butyl-2-[(2,2- | 361.43; | |
| | difluorocyclopropyl)methoxy ]phen | 362.4 | |
| | yl]-2,3-dimethyl-1H-pyridin-4-one | | |
| 136 | 2-[4-tert-butyl-2-[(2,2- | 347.4; | |
| | difluorocyclopropyl)methoxy lphen | 348.4 | |
| | yl]-6-methyl-1H-pyridin-4-one | | |
| 137 | 6-[2-(3,4-difluoro-2-methyl- | 424.36; | ¹H NMR (400 MHz, CD₃OD) δ 8.51 (s, |
| | phenoxy)-4-mcthyl-5- | 425.2 | 1H), 7.18-7.09 (m, 2H), 6.91 (m, 1H), |
| | (trifluoromethyl)-3-pyridyl]-2,3- | | 2.66 (m, 3H), 2.40 (s, 3H), 2.29 (d, J = |
| | dimethyl-1H-pyridin-4-one | | 2.2 Hz, 3H), 2.03 (d, J = 2.2 Hz, 3H). |
| 138 | 2-[2-(4,4-difluoroazepan-1-yl)-5- | 401.37; | ¹H NMR (400 MHz, CD₃OD) δ 7.73 (s, |
| | methyl-6-(trifluoromethyl)-3- | 402.2 | 1H), 6.89 (d, J = 2.4 Hz, 1H), 6.85-6.74 |
| | pyridyl]-6-methyl-1H-pyridin-4- | | (m, 1H), 3.63-3.51 (m, 2H), 3.19 (t, J = |
| | one | | 6.1 Hz, 2H), 2.56 (s, 3H), 2.46-2.36 (m, |
| | | | 3H), 2.36-2.19 (m, 2H), 2.09-1.89 (m, |
| | | | 2H), 1.88-1.73 (m, 2H). |
| 139 | 2-[2-(4,4-difluoroazepan-1-yl)-6- | 387.35; | ¹H NMR (400 MHz, DMSO-d₆) δ 7.97 |
| | (trifluoromethyl)-3-pyridyl]-6- | 388.3 | (d, J = 7.6 Hz, 1H), 7.35 (d, J = 7.7 Hz, |
| | methyl-1H-pyridin-4-one | | 1H), 7.22 (q, J = 2.5 Hz, 1H), 7.13 (d, |
| | | | J = 3.2 Hz, 1H), 3.53-3.49 (m, 2H), 3.09 |
| | | | (t, .J = 6.0 Hz, 2H), 2.59 (s, 3H), 2.38- |
| | | | 2.16 (m, 2H), 2.09-1.86 (m, 2H), 1.84- |
| | | | 1.54 (m, 2H). |
| 140 | 2-[2-(3,4-difluoro-2-methyl- | 378.37; | ¹H NMR (400 MHz, DMSO-d₆) δ 8.82 |
| | phenoxy)-3-quinolyl]-6-methyl- | 379.2 | (s, 1H), 8.08 (dd, J = 8.1, 1.5 Hz, 1H), |
| | 1H-pyridin-4-one | | 7.78 (ddd, J = 8.4, 6.9, 1.5 Hz, 1H), 7.68 |
| | | | (d, J = 8.2 Hz, 1H), 7.60 (ddd, J = 8.2, |
| | | | 6.9, 1.3 Hz, 1H), 7.48 (d, J = 2.3 Hz, 1H), |
| | | | 7.40 (q, J = 9.4 Hz, 1H), 7.25 (ddd, J = |
| | | | 9.3, 4.4, 1.9 Hz, 1H), 7.08 (s, 1H), 2.64 |
| | | | (s, 3H), 2.06 (d, J = 2.0 Hz, 3H). |
| 141 | 6-[2-(3,4-difluoro-2-methyl- | 392.4; | ¹H NMR (400 MHz, DMSO-d₆) δ 8.81 |
| | phenoxy)-3-quinolyl]-2,3- | 393.2 | (s, 1H), 8.09 (dd, J = 8.1, 1.4 Hz, 1H), |
| | dimethyl-1H-pyridin-4-one | | 7.79 (ddd, J = 8.5, 6.8, 1.5 Hz, 1H), 7.69 |
| | | | (d, J = 8.4 Hz, 1H), 7.62 (ddd, J = 8.1, |
| | | | 6.8, 1.3 Hz, 1H), 7.55 (s, 1H), 7.41 (q, J = |
| | | | 9.4 Hz, 1H), 7.24 (ddd, J = 9.2, 4.4, 1.9 |
| | | | Hz, 1H), 2.67 (s, 3H), 2.20 (s, 3H), 2.07 |
| | | | (d, J = 2.0 Hz, 3H). |
| 142 | 2,3-dimethyl-6-[2- | 418.38; | ¹H NMR (400 MHz, CD₃OD) δ 8.96 (s, |
| | (trifluoromethyl)-5-[4- | 419.2 | 0.25H), 8.87 (s, 0.75H), 7.93 (s, 1H), |
| | (trifluoromethyl)cyclohexyl]-4- | | 6.99 (s, 1H), 2.82-2.69 (m, 1H), 2.64 (s, |
| | pyridyl]-1H-pyridin-4-one | | 3H), 2.52-2.36 (m, 1H), 2.29 (s, 3H), |
| | (cis/trans mixture) | | 2.13-1.99 (m, 2H), 1.99-1.81 (m, 2H), |
| | | | 1.80-1.63 (m, 3H), 1.43-1.26 (m, 1H). |
| 143 | 6-[2-(4,4-difluoroazepan-1-yl)-6- | 401.37; | ¹H NMR (400 MHz, CD₃OD) δ 7.77 (d, |
| | (trifluoromethyl)-3-pyridyl]-2,3- | 402.4 | J = 7.6 Hz, 1H), 7.19 (d, J = 7.6 Hz, 1H), |
| | dimethyl-1H-pyridin-4-one | | 6.41 (s, 1H), 3.63-3.56 (m, 2H), 3.30- |
| | | | 3.26 (m, 2H, obscured by solvent peak), |
| | | | 2.39 (s, 3H), 2.34-2.18 (m, 2H), 2.06 (s, |
| | | | 3H), 2.03-1.92 (m, 2H), 1.86-1.76 (m, |
| | | | 2H). |
| 144 | 6-[2-(4,4-difluoroazepan-1-yl)-3- | 383.43; | 1H NMR (400 MHz, CD₃OD) δ 8.13 (s, |
| | quinolyl]-2,3-dimethyl-1H- | 384.3 | 1H), 7.81-7.72 (m, 2H), 7.65 (t, J = 7.5 |
| | pyridin-4-one | | Hz, 1H), 7.35 (t, J = 7.5 Hz, 1H), 6.54 (s, |
| | | | 1H), 3.67-3.60 (m, 2H), 3.43 (t, J = 6.1 |
| | | | Hz, 2H), 2.41 (s, 3H), 2.34-2.20 (m, |
| | | | 2H), 2.09 (s, 3H), 2.07-1.97 (m, 2H), |
| | | | 1.85-1.75 (m, 2H). |
| 146 | 2-[2- | 255.31; | ¹H NMR (400 MHz, DMSO-d₆) δ 13.36 |
| | (cyclopropylmethoxy)phenyl]-6- | 256.3 | (s, 1H), 7.58-7.50 (m, 2H), 7.21 (d, 1H), |
| | methyl-1H-pyridin-4-one | | 7.17-7.14 (m, 1H), 7.12 (d, 1H), 7.02 (s, |
| | | | 1H), 3.94 (d, 2H), 2.58 (s, 3H), 1.21- |
| | | | 1.11 (m, 1H), 0.56-0.46 (m, 2H), 0.36- |
| | | | 0.26 (m, 2H). |
| 147 | 6-[4-(3,4-difluoro-2-methyl- | 410.34; | |
| | phenoxy)-6-(trifluoromethyl)-3- | 411.3 | |
| | pyridyl]-2,3-dimethyl-1H-pyridin- | | |
| | 4-one | | |
| 148 | 2-(2-cyclopropylphenyl)-6-methyl- | 225.29; | |
| | 1H-pyridin-4-one | 226.07 | |
| 149 | 2-[4-(3,3-difluorocyclobutyl)-2-[4- | 425.43; | ¹H NMR (500 MHz, DMSO-d₆) δ 14.18 |
| | (trifluoromethyl)cyclohexyl]pheny | 426.3 | (s, 1H), 7.43-7.34 (m, 2H), 7.33 (s, 1H), |
| | 1]-6-methyl-1H-pyridin-4-one | | 7.16 (d, J = 2.4 Hz, 1H), 7.11 (d, J = 2.5 |
| | | | Hz, 1H), 3.54 (qt, J = 9.2, 4.6 Hz, 1H), |
| | | | 3.13-3.01 (m, 2H), 2.78-2.63 (m, 2H), |
| | | | 2.59 (s, 3H), 2.48 (s, 1H), 1.91 (dd, J = |
| | | | 10.5, 5.7 Hz, 2H), 1.64 (t, J = 10.9 Hz, |
| | | | 6H). |
| 150 | 6-[4-tert-butyl-2-(4-fluoro-2- | 410.48; | ¹H NMR (500 MHz, DMSO-d₆) δ 12.71 |
| | methoxy-phenoxy)-6-methyl- | 411.3 | (br s, 1H), 7.08-7.01 (m, 2H), 6.94 (dd, |
| | phenyl]-2,5-dimethyl-1H- | | J = 8.8, 5.8 Hz, 1H), 6.74 (td, J = 8.5, 2.9 |
| | pyrimidin-4-one | | Hz, 1H), 6.51 (s, 1H), 3.71 (s, 3H), 2.33 |
| | | | (s, 3H), 2.11 (s, 3H), 1.73 (s, 3H), 1.19 |
| | | | (s, 9H). |
| 151 | 2-[4-tert-butyl-2-(4-fluoro-2- | 409.49; | ¹H NMR (500 MHz, DMSO-d₆) δ 13.05 |
| | methoxy-phenoxy)-6-methyl- | 410.4 | (br s, 1H), 7.16 (s, 1H), 7.08-7.03 (m, |
| | phenyl]-3,6-dimethyl-1H-pyridin- | | 1H), 7.03-6.96 (m, 1H), 6.81-6.72 (m, |
| | 4-one | | 2H), 6.56 (s, 1H), 3.70 (s, 3H), 2.41 (s, |
| | | | 3H), 2.08 (s, 3H), 1.80 (d, J = 1.7 Hz, |
| | | | 3H), 1.21 (s, 9H). |
| 152 | ethyl 6-[4-tert-butyl-2-(4-fluoro-2- | 467.53; | ¹H NMR (400 MHz, DMSO-d₆) δ 11.58 |
| | methoxy-phenoxy)-6-methyl- | 468.2 | (s, 1H), 7.13-7.02 (m, 3H), 6.78 (td, J = |
| | phenyl]-2-methyl-4-oxo-1H- | | 8.5, 2.8 Hz, 1H), 6.49 (s, 1H), 6.05-6.01 |
| | pyridine-3-carboxylate | | (m, 1H), 4.21 (q, J = 7.1 Hz, 2H), 3.74 |
| | | | (s, 3H), 2.25-2.18 (m, 6H), 1.25 (t, J = |
| | | | 7.1 Hz, 3H), 1.18 (s, 9H). |
| 153 | 6-[4-tert-butyl-2-(4-fluoro-2- | 409.49; | ¹H NMR (400 MHz, CDCl₃) δ 8.74 (br. |
| | methoxy-phenoxy)-6-methyl- | 410.3 | s., 1H), 7.07-6.96 (m, 2H), 6.79-6.64 |
| | phenyl]-2,3-dimethyl-1H-pyridin- | | (m, 2H), 6.57 (s, 1H), 6.37 (br. s., 1H), |
| | 4-one | | 3.78 (s, 3H), 2.34 (s, 3H), 2.27 (s, 3H), |
| | | | 2.05 (s, 3H), 1.20 (s, 9H). |
| 154 | 2-[4-tert-butyl-2-(4-fluoro-2- | 395.47; | ¹H NMR (400 MHz, DMSO-d₆) δ 8.49 |
| | methoxy-phenoxy)-6-methyl- | 396.4 | (s, 1H), 7.25 (s, 1H), 7.15 (d, J = 1.7 Hz, |
| | phenyl]-5-methyl-1H-pyridin-4- | | 1H), 7.13-7.04 (m, 2H), 6.78 (td, J = |
| | one | | 8.5, 2.9 Hz, 1H), 6.52 (d, J = 1.7 Hz, 1H), |
| | | | 3.71 (s, 3H), 2.20 (s, 3H), 2.16 (s, 3H), |
| | | | 1.18 (s, 9H). |
| 155 | 2-[6-tert-butyl-4-[4- | 392.46; | |
| | (trifluoromethyl)cyclohexyl]-3- | 393.3 | |
| | pyridyl]-6-methyl-1H-pyridin-4- | | |
| | one | | |
| 156 | 2-[2-(4,4-difluoroazepan-1-yl)-5- | 387.35; | ¹H NMR (400 MHz, DMSO-d₆) δ 14.49 |
| | (trifluoromethyl)-3-pyridyl]-6- | 388.3 | (s, 1H), 8.65 (d, J = 2.4 Hz, 1H), 8.11 (d, |
| | methyl-1H-pyridin-4-one | | J = 2.4 Hz, 1H), 7.19 (dd, 2H), 3.17 (s, |
| | | | 2H), 3.09 (t, J = 5.9 Hz, 2H), 2.62 (s, |
| | | | 3H), 2.38-2.23 (m, 2H), 2.04-1.88 (m, |
| | | | 2H), 1.81-1.72 (m, 2H). |
| 157 | 2-[2-(4,4-difluoro-1-piperidyl)-5- | 373.32; | ¹H NMR (400 MHz, DMSO-d₆) δ 8.72 |
| | (trifluoromethyl)-3-pyridyl]-6- | 374.1 | (dd, J = 2.4, 1.0 Hz, 1H), 8.21 (d, J = 2.4 |
| | methyl-1H-pyridin-4-one | | Hz, 1H), 7.40 (d, J = 2.4 Hz, 1H), 7.10 |
| | | | (s, 1H), 3.39-3.32 (m, 4H), 2.61 (s, 3H), |
| | | | 2.06-1.90 (m, 4H). |
| 158 | 2-[4-tert-butyl-2-(4-fluoro-2- | 395.47; | ¹H NMR (400 MHz, CD₃OD) δ 7.10 (s, |
| | methoxy-phenoxy)-6-methyl- | 396.0 | 1H), 6.94 (dd, J = 8.8, 5.6 Hz, 1H), 6.87 |
| | phenyl]-6-methyl-1H-pyridin-4- | | (dd, J = 10.4, 2.8 Hz, 1H), 6.69-6.63 (m, |
| | one | | 1H), 6.63 (s, 1H), 6.32 (d, J = 2.2 Hz, |
| | | | 1H), 6.30-6.26 (m, 1H), 3.75 (s, 3H), |
| | | | 2.34 (s, 3H), 2.23 (s, 3H), 1.22 (s, 9H). |
| 159 | 2-[2-fluoro-6-[2-methoxy-4- | 477.33; | ¹H NMR (400 MHz, CDCl₃) δ 7.48 (t, J = |
| | (trifluoromethoxy)phenoxy]-3- | 478.0 | 8.4 Hz, 1H), 7.19 (d, J = 8.3 Hz, 1H), |
| | (trifluoromethyl)phenyl]-6-methyl- | | 6.95-6.83 (m, 2H), 6.53 (br d, J = 8.8 |
| | 1H-pyridin-4-one | | Hz, 2H), 6.24 (br s, 1H), 3.81 (s, 3H), |
| | | | 2.32 (s, 3H). |
| 160 | 2-[4-tert-butyl-2-(4-fluoro-2- | 367.41; | |
| | hydroxy-phenoxy)-6-methyl- | 368.0 | |
| | phenyl]-1H-pyridin-4-one | | |
| 161 | 6-[2-(3,4-difluoro-2-methyl- | 518.44; | ¹H NMR (400 MHz, CD₃OD) δ 8.47 (s, |
| | phenoxy)-4-methyl-5- | 519.5 | 1H), 8.26 (d, J = 2.8 Hz, 1H), 8.23 (d, |
| | (trifluoromethyl)-3-pyridyl]-3-(3- | | J = 2.8 Hz, 1H), 7.15 (app q, J = 9.2 Hz, |
| | methoxypyrazin-2-yl)-2-methyl- | | 1H), 6.98-6.91 (m, 1H), 6.63 (s, 1H), |
| | 1H-pyridin-4-one | | 3.98 (s, 3H), 2.49 (s, 3H), 2.21 (s, 3H), |
| | | | 2.08 (d, J = 2.2 Hz, 3H |
| 162 | 6-[2-(3,4-difluoro-2-methyl- | 504.45; | ¹H NMR (400 MHz, CD₃OD) δ 8.52 (s, |
| | phenoxy)-4-methyl-5- | 505.37 | 1H), 7.72 (s, 1H), 7.22-7.11 (m, 2H), |
| | (trifluoromethyl)-3-pyridyl]-3- | | 6.98-6.90 (m, 1H), 3.89 (s, 3H), 2.71 (s, |
| | (1,2-dimethylimidazol-4-yl)-2- | | 3H), 2.67 (s, 3H), 2.49-2.45 (m, 3H), |
| | methyl-1H-pyridin-4-one | | 2.06 (d, J = 2.1 Hz, 3H) |
| 163 | 6-[2-(3,4-difluoro-2-methyl- | 488.41; | ¹H NMR (400 MHz, CD₃OD) δ 9.24 (s, |
| | phenoxy)-4-mcthyl-5- | 489.1 | 1H), 8.84 (d, J = 5.3 Hz, 1H), 8.45 (s, |
| | (trifluoromethyl)-3-pyridyl]-2- | | 1H), 7.73 (d, J = 6.6 Hz, 1H), 7.15 (q, |
| | methyl-3-pyrimidin-4-yl-1H- | | J = 9.3 Hz, 1H), 6.96-6.89 (m, 1H), 6.61 |
| | pyridin-4-one | | (s, 1H), 2.47 (s, 3H), 2.36 (s, 3H), 2.07 |
| | | | (d, J = 2.3 Hz, 3H) |
| 164 | 6-[2-(3,4-difluoro-2-methyl- | 492.4; | ¹H NMR (400 MHz, CD₃OD) δ 8.46 (s, |
| | phenoxy)-4-methyl-5- | 493.5 | 1H), 7.15 (app q, J = 9.3 Hz, 1H), 6.93 |
| | (trifluoromethyl)-3-pyridyl]-2- | | (ddd, J = 9.2, 4.2, 2.0 Hz, 1H), 6.61 (s, |
| | methyl-3-(5-methyl-1,3,4- | | 1H), 2.62 (s, 3H), 2.50-2.46 (m, 6H), |
| | oxadiazol-2-yl)-1H-pyridin-4-one | | 2.06 (d, J = 2.1 Hz, 3H) |
| 165 | 6-[5-(3,4-difluoro-2-methyl- | 492.4: | ¹H NMR (400 MHz, CD₃OD) δ 8.07 (s, |
| | phenoxy)-3-mcthyl-2- | 493.6 | 1H), 7.17 (q, J = 9.3 Hz, 1H), 6.88 (ddd, |
| | (trifluoromethyl)-4-pyridyl]-2- | | J = 9.1, 4.0, 2.1 Hz, 1H), 6.56 (s, 1H), |
| | methyl-3-(2-methyltetrazol-5-yl)- | | 4.45 (s, 3H), 2.45 (q, J = 1.8 Hz, 3H), |
| | 1H-pyridin-4-one | | 2.31 (s, 3H), 2.16 (d, J = 2.2 Hz, 3H) |
| 166 | 6-[2-(3,4-difluoro-2-methyl- | 490.43; | ¹H NMR (400 MHz, CD₃OD) δ 8.53 (s, |
| | phenoxy)-4-methyl-5- | 491.5 | 1H), 7.99 (s, 1H), 7.77 (s, 1H), 7.31 (s, |
| | (trifluoromethyl)-3-pyridyl]-2- | | 1H), 7.16 (app q, J = 9.2 Hz, 1H), 6.94 |
| | methyl-3-(1-methylpyrazol-4-yl)- | | (ddd, J = 9.1, 4.1, 2.1 Hz, 1H), 3.99 (s, |
| | 1H-pyridin-4-one | | 3H), 2.71 (s, 3H), 2.48-2.43 (m, 3H), |
| | | | 2.06 (d, J = 2.1 Hz, 3H) |
| 167 | 6-[2-(3,4-difluoro-2-methyl- | 492.4; | ¹H NMR (400 MHz, CD₃OD) δ 8.45 (s, |
| | phenoxy)-4-methyl-5- | 493.5 | 1H), 7.14 (q, J = 9.2 Hz, 1H), 6.93 (ddd, |
| | (trifluoromethyl)-3-pyridyl]-2- | | J = 9.2, 4.2, 2.0 Hz, 1H), 6.58 (s, 1H), |
| | methyl-3-(5-methyl-1,2,4- | | 2.67 (s, 3H), 2.47 (s, 3H), 2.38 (s, 3H), |
| | oxadiazol-3-yl)-1H-pyridin-4-one | | 2.06 (d, J = 2.1 Hz, 3H) |
| 168 | 6-[2-(3,4-difluoro-2-methyl- | 491.41; | ¹H NMR (400 MHz, CD₃OD) δ 8.43 (s, |
| | phenoxy)-4-methyl-5- | 492.2 | 1H), 7.78 (s, 1H), 7.14 (app q, J = 9.2 Hz, |
| | (trifluoromethyl)-3-pyridyl]-2- | | 1H), 6.92 (ddd, J = 9.2, 4.3, 2.1 Hz, 1H), |
| | methyl-3-(4-methyloxazol-2-yl)- | | 6.66 (br s, 1H), 2.61-2.31 (m, 6H), 2.25 |
| | 1H-pyridin-4-one | | (s, 3H), 2.06 (d, J = 2.1 Hz, 3H) |
| 169 | 6-[2-(3,4-difluoro-2-methyl- | 478.37; | ¹H NMR (400 MHz, CD₃OD) δ 9.10 (s, |
| | phenoxy)-4-methyl-5- | 479.5 | 1H), 8.46 (s, 1H), 7.15 (q, J = 9.3 Hz, |
| | (trifluoromethyl)-3-pyridyl]-2- | | 1H), 6.94 (ddd, J = 9.2, 4.2, 2.1 Hz, 1H), |
| | methyl-3-(1,3,4-oxadiazol-2-yl)- | | 6.61 (br s, 1H), 2.51-2.46 (m, 6H), 2.07 |
| | 1H-pyridin-4-one | | (d, J = 2.1 Hz, 3H) |
| 170 | 6-[2-(4-fluoro-2-methyl-phenoxy)- | 474.41; | ¹H NMR (400 MHz, CD₃OD) δ 8.51 (s, |
| | 4-methyl-5-(trifluoromethyl)-3- | 475.4 | 1H), 7.16 (s, 1H), 7.10 (dd, J = 8.9, 4.9 |
| | pyridyl]-2-methyl-3-(2- | | Hz, 1H), 7.04 (dd, J = 9.2, 3.1 Hz, 1H), |
| | methyltetrazol-5-yl)-1H-pyridin-4- | | 6.97 (td, J = 8.5, 3.1 Hz, 1H), 4.50 (s, |
| | one | | 3H), 2.58 (s, 3H), 2.51-2.46 (m, 3H), |
| | | | 2.10 (s, 3H) |
| 171 | 6-[2-(3,4-difluoro-2-methyl- | 492.4; | ¹H NMR (400 MHz, CD₃OD) δ 8.51 (s, |
| | phenoxy)-4-methyl-5- | 493.5 | 1H), 7.15 (app q, J = 9.2 Hz, 1H), 7.00 |
| | (trifluoromethyl)-3-pyridyl]-2- | | (s, 1H), 6.95 (ddd, J = 9.1, 4.2, 2.1 Hz, |
| | methyl-3-(2-methyltetrazol-5-yl)- | | 1H), 4.49 (s, 3H), 2.51 (s, 3H), 2.50- |
| | 1H-pyridin-4-one | | 2.47 (m, 3H), 2.07 (d, J = 2.1 Hz, 3H) |
| 172 | 6-12-(3,4-difluoro-2-methyl- | 491.41; | ¹H NMR (400 MHz, CD₃OD) δ 8.52 (s, |
| | phenoxy)-4-methyl-5- | 492.5 | 1H), 8.07 (s, 1H), 7.21-7.10 (m, 2H), |
| | (trifluoromethyl)-3-pyridyl]-2- | | 6.94 (ddd, J = 9.2, 4.2, 2.0 Hz, 1H), 4.28 |
| | methyl-3-(2-methyltriazol-4-yl)- | | (s, 3H), 2.73 (s, 3H), 2.49-2.44 (m, 3H), |
| | 1H-pyridin-4-one | | 2.06 (d, J = 2.1 Hz, 3H) |
| 173 | 6-[2-(3,4-difluoro-2-methyl- | 492.4; | ¹H NMR (400 MHz, CD₃OD) δ 8.45 (s, |
| | phenoxy)-4-methyl-5- | 493.5 | 1H), 7.14 (app q, J = 9.3 Hz, 1H), 6.97- |
| | (trifluoromethyl)-3-pyridyl]-2- | | 6.89 (m, 1H), 6.62 (br s, 1H), 2.50 (s, |
| | methyl-3-(3-methyl-1,2,4- | | 3H), 2.48-2.45 (m, 6H), 2.06 (d, J = 2.1 |
| | oxadiazol-5-yl)-1H-pyridin-4-one | | Hz, 3H) |
| 174 | 6-[2-(3,4-difluoro-2-methyl- | 477.38; | ¹H NMR (400 MHz, CD₃OD) δ 8.50 (s, |
| | phenoxy)-4-methyl-5- | 478.5 | 1H), 8.23 (s, 1H), 7.52 (s, 1H), 7.20- |
| | (trifluoromethyl)-3-pyridyl]-2- | | 7.09 (m, 2H), 6.94 (ddd, J = 9.2, 4.2, 2.1 |
| | methyl-3-oxazol-2-yl-1H-pyridin- | | Hz, 1H), 2.77 (s, 3H), 2.48-2.43 (m, |
| | 4-one | | 3H), 2.05 (d, J = 2.2 Hz, 3H) |
| 175 | 6-[2-(3,4-difluoro-2-methyl- | 491.41; | ¹H NMR (400 MHz, CD₃OD) δ 8.43 (s, |
| | phenoxy)-4-methyl-5- | 492.1 | 1H), 7.14 (q, J = 9.3 Hz, 1H), 6.99 (s, |
| | (trifluoromethyl)-3-pyridyl]-2- | | 1H), 6.92 (ddd, J = 9.2, 4.2, 2.1 Hz, 1H), |
| | methyl-3-(5-methyloxazol-2-yl)- | | 6.66 (br s, 1H), 2.61-2.32 (m, 9H), 2.06 |
| | 1H-pyridin-4-one | | (d, J = 2.1 Hz, 3H) |
| 176 | 6-[2-(3,4-difluoro-2-methyl- | 491.41; | ¹H NMR (400 MHz, CD₃OD) δ 7.42-7.29 |
| | phenoxy)-4-methyl-5- | 492.4 | (m, 5H), 7.18 (s, 1H), 6.48 (s, 1H), |
| | (trifluoromethyl)-3-pyridyl]-2- | | 5.24 (s, 2H), 2.43 (s, 3H), 2.32 (s, 3H) |
| | methyl-3-(3-methylisoxazol-5-yl)- | | |
| | 1H-pyridin-4-one | | |
| 177 | 6-[2-(4,4-difluoroazcpan-1-yl)-5- | 481.46; | ¹H NMR (400 MHz, CD₃OD) δ 7.91 (s, |
| | methyl-6-(trifluoromethyl)-3- | 482.6 | 1H), 7.75 (s, 1H), 7.70 (s, 1H), 6.87 (s, |
| | pyridyl]-2-methyl-3-(1- | | 1H), 3.98 (s, 3H), 3.63-3.56 (m, 2H), |
| | methylpyrazol-4-yl)-1H-pyridin-4- | | 3.26 (t, J = 6.1 Hz, 2H), 2.58 (s, 3H), |
| | one | | 2.41 (q, J = 2.1 Hz, 3H), 2.38-2.23 (m, |
| | | | 2H), 2.06-1.92 (m, 2H), 1.89-1.79 (m, |
| | | | 2H) |
| 178 | 6-[2-(4,4-difluoroazepan-1-yl)-5- | 483.44; | ¹H NMR (400 MHz, CD₃OD) δ 7.80 (s, |
| | methyl-6-(trifluoromethyl)-3- | 484.2 | 1H), 6.93 (s, 1H), 4.49 (s, 3H), 3.66- |
| | pyridyl]-2-methyl-3-(2- | | 3.59 (m, 2H), 3.29-3.24 (m, 2H), 2.50 |
| | methyltetrazol-5-yl)-1H-pyridin-4- | | (s, 3H), 2.42 (q, J = 2.2 Hz, 3H), 2.39- |
| | one | | 2.25 (m, 2H), 2.07-1.93 (m, 2H), 1.91- |
| | | | 1.81 (m, 2H) |
| 179 | 6-[2-tert-butyl-4-(4,4- | 443.49; | ¹H NMR (400 MHz, CD₃OD) δ 8.70 (s, |
| | difluorocyclohexyl)pyrimidin-5- | 444.1 | 1H), 6.52 (s, 1H), 4.46 (s, 3H), 3.07- |
| | yl]-2-methyl-3-(2-methyltetrazol- | | 2.94 (m, 1H), 2.32 (s, 3H), 2.18-2.05 |
| | 5-yl)-1H-pyridin-4-one | | (m, 4H), 1.96-1.77 (m, 4H), 1.43 (s, 9H) |
| 180 | 3-[6-[2-(3,4-difluoro-2-methyl- | 504.41; | ¹H NMR (400 MHz, CD₃OD) δ 8.50 (s, 1H), |
| | phenoxy)-4-methyl-5- | 505.4 | 7.62 (d, J = 4.1 Hz, 1H), 7.54 (d, J = |
| | (trifluoromethyl)-3-pyridyl]-2- | | 4.1 Hz, 1H), 7.15 (app q, J = 9.3 Hz, |
| | methyl-4-oxo-1H-pyridin-3-yl]- | | 1H), 6.98 (s, 1H), 6.93 (ddd, J = 9.3, 4.3, |
| | 1H-pyrazin-2-one | | 2.0 Hz, 1H), 2.51-2.46 (m, 3H), 2.41 (s, |
| | | | 3H), 2.06 (d, J = 2.1 Hz, 3H) |
| 181 | 6-[5-tert-butyl-2-(4,4- | 445.51; | ¹H NMR (400 MHz, CD₃OD) δ 6.44 (s, |
| | difluorocyclohexyl)-4-methyl- | 446.1 | 1H), 4.45 (s, 3H), 4.26-4.13 (m, 1H), |
| | pyrazol-3-yl]-2-methyl-3-(2- | | 2.29 (s, 3H), 2.27-2.14 (m, 4H), 2.12 (s, |
| | methyltetrazol-5-yl)-1H-pyridin-4- | | 3H), 2.02-1.84 (m, 4H), 1.36 (s, 9H) |
| | one | | |
| 182 | ethyl 2-[2-(2-ethyl-4-fluoro- | 464.41; | ¹H NMR (400 MHz, CD₃OD) δ 8.51 (s, |
| | phenoxy)-5-(trifluoromethyl)-3- | 465.2 | 1H), 8.21 (s, 1H), 7.15-7.02 (m, 2H), |
| | pyridyl]-6-methyl-4-oxo-1H- | | 6.97 (td, J = 8.4, 3.1 Hz, 1H), 6.50 (s, |
| | pyridine-3-carboxylate | | 1H), 4.11 (q, J = 7.1 Hz, 2H), 2.51-2.36 |
| | | | (m, 5H), 1.05 (t, J = 7.6 Hz, 3H), 0.97 (t, |
| | | | J = 7.1 Hz, 3H) |
| 183 | ethyl 2-[2-(3,4-difluoro-2-methyl- | 575.48; | ¹H NMR (400 MHz, CD₃OD) δ 8.25 (s, |
| | phenoxy)-6-methyl-5- | 576.6 | 1H), 7.66 (dd, J = 6.9, 2.1 Hz, 1H), 7.57 |
| | (trifluoromethyl)-3-pyridyl]-6- | | (dd, J = 6.5, 2.1 Hz, 1H), 7.17 (app q, |
| | methyl-4-oxo-5-(2-oxo-1H- | | J = 9.3 Hz, 1H), 6.91 (ddd, J = 9.1, 4.2, |
| | pyridin-3-yl)-1H-pyridine-3- | | 2.1 Hz, 1H), 6.55 (t, J = 6.7 Hz, 1H), |
| | carboxylate | | 4.17 (q, J = 7.1 Hz, 2H), 2.53 (q, J = 1.7 |
| | | | Hz, 3H), 2.38 (s, 3H), 2.07 (d, J = 2.1 |
| | | | Hz, 3H), 0.98 (t, J = 7.1 Hz, 3H) |
| 184 | ethyl 2-[2-(3,4-difluoro-2-methyl- | 575.48; | ¹H NMR (400 MHz, CD₃OD) δ 8.17 (s, |
| | phenoxy)-6-methyl-5- | 576.5 | 1H), 7.82 (dd, J = 9.1, 7.0 Hz, 1H), 7.17 |
| | (trifluoromethyl)-3-pyridyl]-6- | | (q, .J = 9.3 Hz, 1H), 6.92 (ddd, J = 9.2, |
| | methyl-4-oxo-5-(6-oxo-1H- | | 4.2, 2.0 Hz, 1H), 6.71 (d, J = 9.0 Hz, |
| | pyridin-2-yl)-1H-pyridine-3- | | 1H), 6.63 (d, J = 7.0 Hz, 1H), 4.12 (q, |
| | carboxylate | | J = 7.1 Hz, 2H), 2.56-2.45 (m, 3H), 2.36 |
| | | | (s, 3H), 2.07 (d, J = 2.2 Hz, 3H), 1.00 |
| | | | (t, J = 7.1 Hz, 3H) |
| 185 | 6-[5-(3,4-difluoro-2-methoxy- | 493.38; | ¹H NMR (400 MHz, CDCl₃) δ 13.15 |
| | phenoxy)-3-methyl-2- | 494.11 | (br, s, 1H), 7.99 (s, 1H), 7.91 (s, 1H), |
| | (trifluoromethyl)-4-pyridyl]-2- | | 7.39 (s, 1H), 7.03 (s, 1H), 6.90-6.82 (m, |
| | methyl-3-oxazol-2-yl-1H-pyridin- | | 2H), 3.89 (d, J = 1.8 Hz, 3H), 2.97 (s, |
| | 4-one | | 3H), 2.35 (s, 3H). |
| 186 | 5-[6-[6-(3,3-difluorocyclobutyl)-4- | 520.49; | ¹H NMR (400 MHz, CD₃OD) δ 8.91-8.24 |
| | [4-(trifluoromethyl)cyclohexyl]-3- | 521.5 | (br s, 2H) overlaps with 8.64 (s, |
| | pyridyl]-2-methyl-4-oxo-1H- | | 1H), 7.53 (s, 1H), 7.06 (s, 1H), 3.75- |
| | pyridin-3-yl]-1H-pyrimidin-2-one | | 3.63 (m, 1H), 3.09-2.87 (m, 3H), 2.84- |
| | (cis isomer) | | 2.74 (m, 1H), 2.63 (s, 3H), 2.46 (s, |
| | | | OH), 2.16-2.05 (m, 2H), 1.92-1.78 |
| | | | (m, 3H), 1.78-1.67 (m, 5H) |
| 187 | 6-[6-(3,3-difluorocyclobutyl)-4-[4- | 519.51; | ¹H NMR (400 MHz, CD₃OD) δ 8.69 (s, |
| | (trifluoromethyl)cyclohexyl]-3- | 520.5 | 1H), 7.75 (dd, J = 7.0, 2.1 Hz, 1H), 7.65 |
| | pyridyl]-2-methyl-3-(2-oxo-1H- | | (dd, J = 6.5, 2.1 Hz, 1H), 7.56 (s, 1H), |
| | pyridin-3-yl)-1H-pyridin-4-one | | 7.20 (s, 1H), 6.61 (t, J = 6.7 Hz, 1H), |
| | (cis isomer) | | 3.77-3.64 (m, 1H), 3.09-2.90 (m, |
| | | | 4H), 2.85-2.75 (m, 1H), 2.53 (s, 3H), |
| | | | 2.50-2.39 (m, 1H), 2.12-2.05 (m, |
| | | | 2H), 1.91-1.68 (m, 6H) |
| 188 | 6-[6-(3,3-difluorocyclobutyl)-4-[4- | 533.53; | ¹H NMR (400 MHz, CD₃OD) δ 8.70 (s, |
| | (trifluoromethyl)cyclohexyl]-3- | 534.6 | 1H), 7.90 (dd, J = 8.4, 7.3 Hz, 1H), 7.56 |
| | pyridyl]-3-(6-methoxy-2-pyridyl)- | | (s, 1H), 7.24 (d, J = 1.2 Hz, 2H), 6.96 |
| | 2-methyl-1H-pyridin-4-one (cis | | (dd, J = 8.5, 0.8 Hz, 1H), 3.96 (s, 3H), |
| | isomer) | | 3.77-3.63 (m, 1H), 3.08-2.90 (m, |
| | | | 4H), 2.86-2.77 (m, 1H), 2.59 (s, 3H), |
| | | | 2.53-2.40 (m, 1H), 2.14-2.06 (m, |
| | | | 2H), 1.94-1.69 (m, 6H) |
| 189 | 6-[6-(3,3-difluorocyclobutyl)-4-[4- | 493.47; | ¹H NMR (400 MHz, CD₃OD) δ 8.71 (s, |
| | (trifluoromethyl)cyclohexyl]-3- | 494.5 | 1H), 8.36 (d, J = 0.9 Hz, 1H), 7.65 (d, |
| | pyridyl]-2-methyl-3-oxazol-2-yl- | | J = 1.0 Hz, 1H), 7.61 (s, 1H), 7.41 (s, |
| | 1H-pyridin-4-one (cis isomer) | | 1H), 3.72 (qd, J = 8.6, 2.6 Hz, 1H), |
| | | | 3.11-2.92 (m, 4H), 3.02 (s, 3H), 2.90-2.78 |
| | | | (m, 1H), 2.52-2.34 (m, 1H), 2.14-2.02 |
| | | | (m, 2H), 1.90-1.63 (m, 6H) |
| 190 | 6-16-(3,3-difluorocyclobutyl)-4-[4- | 493.47; | ¹H NMR (400 MHz, CD₃OD) δ 8.65 (s, |
| | (trifluoromethyl)cyclohexyl]-3- | 494.5 | 1H), 8.48 (s, 1H), 7.75 (s, 1H), 7.50 (s, |
| | pyridyl]-2-methyl-3-oxazol-5-yl- | | 1H), 7.15 (s, 1H), 3.73-3.61 (m, 1H), |
| | 1H-pyridin-4-one (cis isomer) | | 3.08-2.88 (m, 4H), 2.82 (s, 3H), 2.80- |
| | | | 2.72 (m, 1H), 2.52-2.37 (m, 1H), 2.12- |
| | | | 2.04 (m, 2H), 1.91-1.77 (m, 2H), |
| | | | 1.73 (d, J = 12.8 Hz, 4H) |
| 191 | 6-[6-(3,3-difluorocyclobutyl)-4-[4- | 519.51; | ¹H NMR (400 MHz, CD₃OD) δ 8.69 (s, |
| | (trifluoromethyl)cyclohexyl]-3- | 520.5 | 1H), 7.72 (d, J = 6.6 Hz, 1H), 7.57 (s, |
| | pyridyl]-2-methyl-3-(2-oxo-1H- | | 1H), 7.20 (s, 1H), 6.71 (d, J = 1.6 Hz, |
| | | | 1H), 6.57 (dd, J = 6.7, 1.7 Hz, 1H), |
| | pyridin-4-yl)-1H-pyridin-4-one | | 3.78-3.65 (m, 1H), 3.11-2.89 (m, 4H), |
| | (cis isomer) | | 2.86-2.76 (m, 1H), 2.58 (s, 3H), |
| | | | 2.52-2.40 (m, 1H), 2.13-2.06 (m, 2H), |
| | | | 1.94-1.69 (m, 6H) |
| 192 | 6-[6-[2-(3,4-difluoro-2-methyl- | 530.45; | |
| | phenoxy)-4-methyl-5- | 531.5 | |
| | (trifluoromethyl)-3-pyridyl]-2- | | |
| | methyl-4-oxo-1H-pyridin-3- | | |
| | yl]pyridine-2-carboxamide | | |
| 193 | 6-[6-(3,3-difluorocyclobutyl)-4-[4- | 519.51; | ¹H NMR (400 MHz, CD₃OD) δ 8.72 (s, |
| | (trifluoromethyl)cyclohexyl]-3- | 520.5 | 1H), 7.80-7.72 (m, 2H), 7.62 (s, 1H), |
| | pyridyl]-2-methyl-3-(6-oxo-1H- | | 7.23 (s, 1H), 6.86-6.79 (m, 1H), 3.74 |
| | pyridin-3-yl)-1H-pyridin-4-one | | (pd, J = 8.7, 2.6 Hz, 1H), 3.12-2.90 |
| | (cis isomer) | | (m, 4H), 2.87-2.76 (m, 1H), 2.61 (s, |
| | | | 3H), 2.46 (s, 1H), 2.15-2.04 (m, 2H), |
| | | | 1.93-1.69 (m, 6H) |
| 194 | 6-[6-(3,3-difluorocyclobutyl)-4-[4- | 519.51; | ¹H NMR (400 MHz, CD₃OD) δ 8.78 (s, |
| | (trifluoromethyl)cyclohexyl]-3- | 520.5 | 1H), 8.01 (dd, J = 9.0, 7.0 Hz, 1H), 7.70 |
| | pyridyl]-2-methyl-3-(6-oxo-1H- | | (s, 1H), 7.17 (s, 1H), 6.96-6.88 (m, |
| | pyridin-2-yl)-1H-pyridin-4-one | | 2H), 3.85-3.70 (m, 1H), 3.17-2.93 |
| | (cis isomer) | | (m, 4H), 2.90-2.80 (m, 1H), 2.60 (s, |
| | | | 3H), 2.53-2.40 (m, 1H), 2.15-2.07 |
| | | | (m, 2H), 1.94-1.67 (m, 6H) |
| 195 | 6-[2-(3,4-difluoro-2-methoxy- | 495.7; | ¹H NMR (500 MHz, CD₃OD) δ 7.73 (d, |
| | phenoxy)-5-fluoro-4- | 496.1 | J = 10.3 Hz, 2H), 7.18 (d, J = 5.7 Hz, |
| | (trifluoromethyl)phenyl]-2-methyl- | | 1H), 7.04 (td, J = 9.6, 8.3 Hz, 1H), 6.91 |
| | 3-(1H-pyrazol-3-yl)-1H-pyridin-4- | | (ddd, J = 9.2, 4.8, 2.3 Hz, 1H), 6.75 (s, |
| | one | | 1H), 6.48 (s, 1H), 3.88 (d, J = 1.5 Hz, |
| | | | 3H), 2.46 (s, 3H) |
| 196 | 6-[2-(3,4-difluoro-2-methoxy- | 497.35; | ¹H NMR (400 MHz, CD₃OD) δ 9.07 (s, |
| | phenoxy)-5-fluoro-4- | 498.07 | 1H), 7.73 (d, J = 10.1 Hz, 1H), 7.16 (d, |
| | (trifluoromethyl)phenyl]-2-methyl- | | J = 6.0 Hz, 1H), 7.00 (t, J = 8.9 Hz, |
| | 3-(1,3,4-oxadiazol-2-yl)-1H- | | 1H), 6.90 (m, 1H), 6.71 (s, 1H), 4.56 (s, |
| | pyridin-4-one | | 1H), 3.84 (d, J = 1.4 Hz, 3H), 2.46 (s, |
| | | | 3H) |
| 197 | 2-[2-(3,4-difluoro-2-methoxy- | 509.4; | ¹H NMR (500 MHz, CD₃OD) δ 7.51-7.48 |
| | phenoxy)-5-fluoro-4- | 508.1 | (m, 1H), 7.46 (d, J = 10.1 Hz, 1H), |
| | (trifluoromethyl)phenyl]-6-methyl- | (M-1)⁻ | 6.96 (dd, J = 14.3, 7.4 Hz, 2H), 6.60 (s, |
| | 3-(1-methylpyrazol-3-yl)-1H- | | 1H), 6.47 (s, 1H), 6.20 (s, 1H), 3.80 (s, |
| | pyridin-4-one | | 6H), 2.42 (s, 3H) |
| 198 | 2-[2-(3,4-difluoro-2-methoxy- | 527.46; | ¹H NMR (500 MHz, DMSO-d₆) δ 11.03 |
| | phenoxy)-5-fluoro-4- | 528.2 | (s, 1H), 7.78 (d, J = 10.2 Hz, 1H), 7.29 |
| | (trifluoromethyl)phenyl]-5,6- | | (d, J = 6.0 Hz, 1H), 7.19 (q, J = 9.3 Hz, |
| | dimethyl-3-tetrahydropyran-4-yl- | | 1H), 6.88 (ddd, J = 9.2, 5.0, 2.0 Hz, 1H), |
| | 1H-pyridin-4-one | | 3.81-3.73 (m, 5H), 3.13 (dtd, J = 14.4, |
| | | | 12.2, 11.8, 2.2 Hz, 2H), 2.58 (tt, J = 12.5, |
| | | | 6.2 Hz, 1H), 2.45 (td, J = 12.5, 4.4 Hz, |
| | | | 1H), 2.29 (t, J = 11.8 Hz, 1H), 2.18 (s, |
| | | | 3H), 1.84 (s, 3H), 1.22 (d, J = 13.0 Hz, |
| | | | 1H), 1.01 (d, J = 12.7 Hz, 1H) |
| 199 | 2-[2-(3,4-difluoro-2-methoxy- | 513.43; | ¹H NMR (500 MHz, CD₃OD) δ 7.60-7.52 |
| | phenoxy)-5-fluoro-4- | 512.2 | (m, 1H), 7.18 (dd, J = 17.1, 5.8 Hz, 1H), |
| | (trifluoromethyl)phenyl]-5,6- | (M-1)⁻ | 7.03 (tdd, J = 9.7, 8.3, 3.2 Hz, 1H), |
| | dimethyl-3-tetrahydrofuran-3-yl- | | 6.85 (ddt, J = 9.4, 4.7, 2.3 Hz, 1H), 4.14- |
| | 1H-pyridin-4-one | | 3.99 (m, 2H), 3.89 (td, J = 7.6, 5.5 Hz, |
| | | | 1H), 3.85 (dd, J = 3.0, 1.7 Hz, 3H), 3.78 |
| | | | (ddd, J = 16.2, 8.6, 7.6 Hz, 1H), 2.97 (h, |
| | | | J = 8.7 Hz, 1H), 2.50 (qd, J = 11.8, 10.0, |
| | | | 6.6 Hz, 1H), 2.35 (d, J = 1.9 Hz, 3H), |
| | | | 2.03 (s, 3H), 1.87 (dt, J = 17.4, 8.6 Hz, |
| | | | 1H) |
| 200 | 2-[2-(3,4-difluoro-2-methoxy- | 514.42; | ¹H NMR (400 MHz, CD₃OD) δ 7.54 (d, |
| | phenoxy)-5-fluoro-4- | 515.06 | J = 10.4 Hz, 1H), 7.13 (d, J = 5.8 Hz, |
| | (trifluoromethyl)phenyl]-6-methyl- | | 1H), 7.02 (q, J = 8.5 Hz, 1H), 6.79 (ddd, |
| | 3-morpholino-1H-pyridin-4-one | | J = 9.3, 4.8, 2.4 Hz, 1H), 6.30 (s, 1H), |
| | | | 4.58 (s, 1H), 3.81 (d, J = 1.6 Hz, 3H), |
| | | | 3.50 (t, J = 4.5 Hz, 4H), 3.09 (br. s, 4H), |
| | | | 2.30 (s, 3H) |
| 201 | 2-[2-(3,4-difluoro-2-methoxy- | 497.35; | ¹H NMR (400 MHz, CD₃OD) δ 9.00 (s, |
| | phenoxy)-5-fluoro-4- | 498.0 | 1H), 7.63 (d, J = 9.9 Hz, 1H), 7.04-6.95 |
| | (trifluoromethyl)phenyl]-6-methyl- | | (m, 2H), 6.74-6.66 (m, 1H), 6.53-6.46 |
| | 3-(1,3,4-oxadiazol-2-yl)-1H- | | (m, 1H), 3.79 (d, .J = 1.3 Hz, 3H), 2.44 |
| | pyridin-4-one | | (s, 3H) |
| 202 | ethyl 5-cyclopropyl-2-[2-(3,4- | 490.5; | ¹H NMR (400 MHz, CD₃OD) δ 8.34 (s, |
| | difluoro-2-methyl-phenoxy)-3- | 491.4 | 1H), 7.93 (d, J = 8.0 Hz, 1H), 7.78-7.65 |
| | quinolyl]-6-methyl-4-oxo-1H- | | (m, 2H), 7.57-7.48 (m, 1H), 7.16 (q, J = |
| | pyridine-3-carboxylate | | 9.3 Hz, 1H), 7.01 (br s, 1H), 4.00 (q, J = |
| | | | 7.0 Hz, 2H), 2.54 (s, 3H), 2.08 (s, 3H), |
| | | | 1.54 (br s, 1H), 0.99 (d, J = 8.1 Hz, 2H), |
| | | | 0.84-0.65 (m, 5H) |
| 203 | ethyl 5-cyclopropyl-2-[2-(4,4- | 513.5; | ¹H NMR (400 MHz, CD₃OD) δ 7.50 (s, |
| | difluoroazepan-1-yl)-5-methyl-6- | 514.3 | 1H), 4.07 (q, J = 7.1 Hz, 2H), 3.54 (br s, |
| | (trifluoromethyl)-3-pyridyl]-6- | | 2H), 3.33 (br s, 2H, obscured by water |
| | methyl-4-oxo-1H-pyridine-3- | | peak), 2.48 (s, 3H), 2.35 (q, J = 2.2 Hz, |
| | carboxylate | | 3H), 2.30-2.15 (m, 2H), 2.06-1.92 (m, |
| | | | 2H), 1.87-1.76 (m, 2H), 1.58-1.45 (m, |
| | | | 1H), 1.04-0.93 (m, 5H), 0.73-0.64 (m, |
| | | | 2H) |
| 204 | 2-[2-(3,4-difluoro-2-methoxy- | 528.44; | ¹H NMR (500 MHz, CD₃OD) δ 7.54 (d, |
| | phenoxy)-5-fluoro-4- | | J = 10.1 Hz, 1H), 7.15 (d, J = 5.8 Hz, |
| | (trifluoromethyl)phenyl]-5,6- | | 1H), 7.02 (td, J = 9.6, 8.3 Hz, 1H), 6.79 |
| | | | (ddd, J = 9.3, 4.9, 2.4 Hz, 1H), 3.82 (d, |
| | dimethyl-3-morpholino-1H- | | J = 1.6 Hz, 3H), 3.51 (t, J = 4.7 Hz, 4H), |
| | pyridin-4-one | | 3.14 (s, 4H), 2.35 (d, J = 0.6 Hz, 3H), |
| | | | 2.04 (s, 3H) |
| 205 | 4-16-12-(3,4-difluoro-2-methyl- | 495.4; | ¹H NMR (400 MHz, CD₃OD) δ 8.42 (s, |
| | phenoxy)-4-methyl-5- | 496.4 | 1H), 7.13 (app q, J = 9.2 Hz, 1H), 6.88 |
| | (trifluoromethyl)-3-pyridyl]-2- | | (ddd, J = 9.1, 4.2, 2.1 Hz, 1H), 6.45 (br |
| | methyl-4-oxo-1H-pyridin-3- | | s, 1H), 5.38-5.30 (m, 1H), 4.69 (dd, J = |
| | ylJoxazolidin-2-one | | 10.0, 8.2 Hz, 1H), 4.54-4.46 (m, 1H), |
| | | | 2.49 (s, 3H), 2.41 (br s, J = 1.6 Hz, 3H), |
| | | | 2.03 (d, J = 2.1 Hz, 3H) |
| 206 | 4-[6-[2-(3,4-difluoro-2-methyl- | 495.4; | ¹H NMR (400 MHz, CD₃OD) δ 8.43 (s, |
| | phenoxy)-4-methyl-5- | 496.4 | 1H), 7.13 (app q, J = 9.3 Hz, 1H), 6.88 |
| | (trifluoromethyl)-3-pyridyl]-2- | | (ddd, J = 9.1, 4.2, 2.1 Hz, 1H), 6.49 (s, |
| | methyl-4-oxo-1H-pyridin-3- | | 1H), 5.34 (dd, J = 10.0, 5.9 Hz, 1H), 4.69 |
| | ylloxazolidin-2-one | | (dd, J = 10.0, 8.3 Hz, 1H), 4.50 (dd, J = |
| | | | 8.3, 5.9 Hz, 1H), 2.50 (s, 3H), 2.41 (q, |
| | | | J = 1.4 Hz, 3H), 2.03 (d, J = 2.1 Hz, 3H) |
| 207 | 5-[6-[2-(3,4-difluoro-2-methyl- | 495.4; | ¹H NMR (400 MHz, CD₃OD) δ 8.43 (s, |
| | phenoxy)-4-methyl-5- | 496.5 | 1H), 7.13 (q, J = 9.3 Hz, 1H), 6.90 (ddd, |
| | (trifluoromethyl)-3-pyridyl]-2- | | J = 9.1, 4.1, 2.1 Hz, 1H), 6.45 (s, 1H), |
| | methyl-4-oxo-1H-pyridin-3- | | 6.01 (t, J = 8.8 Hz, 1H), 3.90-3.81 (m, |
| | ylJoxazolidin-2-one | | 1H), 3.80-3.73 (m, 1H), 2.51 (s, 3H), |
| | | | 2.45-2.39 (m, 3H), 2.04 (d, J = 2.1 Hz, |
| | | | 3H) |
| 208 | 5-[6-[2-(3,4-difluoro-2-methyl- | 495.4; | ¹H NMR (400 MHz, CD₃OD) δ 8.43 (s, |
| | phenoxy)-4-methyl-5- | 496.4 | 1H), 7.13 (q, J = 9.2 Hz, 1H), 6.90 (ddd, |
| | (trifluoromethyl)-3-pyridyl]-2- | | J = 9.1, 4.1, 2.0 Hz, 1H), 6.45 (s, 1H), |
| | methyl-4-oxo-1H-pyridin-3- | | 6.01 (t, J = 9.0 Hz, 1H), 3.90-3.81 (m, |
| | ylJoxazolidin-2-one | | 1H), 3.80-3.73 (m, 1H), 2.51 (s, 3H), |
| | | | 2.45-2.39 (m, 3H), 2.04 (d, J = 2.1 Hz, |
| | | | 3H) |

### Separation of Sulfoximine Enantiomers

For Compound 7 and Compound 6, the racemic OBn-precursor [4-benzyloxy-6-[6-(3,3-difluorocyclobutyl)-4-[4-(trifluoromethyl)cyclohexyl]-3-pyridyl]-2-methyl-3-pyridyl]-imino-methyl-oxo-λ6-sulfane (cis cyclohexyl isomer) was purified by chiral SFC (ChiralPak IC column, 250×21.2 mm, 5 μm particle size, isocratic 86% CO₂/14% 3:1 acetonitrile/methanol over 30 minutes, flow rate=70 mL/min, column temperature=40° C.) to provide the separated, OBn-protected sulfoximine enantiomers:

Peak 1 (retention time 18.7 min) was isolated and debenzylated using standard hydrogenation conditions to provide Compound 7.

Peak 2 (retention time 30.0 min) was isolated and debenzylated using standard hydrogenation conditions to provide Compound 6.

For Compound 22 and Compound 21, the racemic OBn-precursor [4-benzyloxy-6-[2-(3,4-difluoro-2-methoxy-phenoxy)-5-fluoro-4-(trifluoromethyl)phenyl]-2-methyl-3-pyridyl]-imino-methyl-oxo-λ6-sulfane was purified by chiral SFC (Lux i-Cellulose-5 column, 250×30 mm, 5 μm particle size, isocratic 88% CO₂/12% methanol (20 nM NH₃) over 13 minutes, flow rate=100 mL/min, column temperature=40° C.) to provide the separated, OBn-protected sulfoximine enantiomers:

Peak 1 (retention time 9.7 min) was isolated and debenzylated using standard hydrogenation conditions to provide Compound 22.

Peak 2 (retention time 11.1 min) was isolated and debenzylated using standard hydrogenation conditions to provide Compound 21.

For Compound 205 and 206, the racemic OBn-precursor 4-[4-benzyloxy-6-[2-(3,4-difluoro-2-methyl-phenoxy)-4-methyl-5-(trifluoromethyl)-3-pyridyl]-2-methyl-3-pyridyl]oxazolidin-2-one was purified by chiral SFC (ChiralPak AS column, 250×21.2 mm, 5 μm particle size, isocratic 77% CO₂/23% methanol (20 nM NH₃) over 16 minutes, flow rate=70 mL/min, column temperature=40° C.) to provide the separated, OBn-protected enantiomers.

Peak 1 (retention time 2.7 min) was isolated and debenzylated using standard hydrogenation conditions to provide 205.

Peak 2 (retention time 11.6 min) was isolated and debenzylated using standard hydrogenation conditions to provide 206.

For Compound 207 and 208, the racemic OBn-precursor 5-[4-benzyloxy-6-[2-(3,4-difluoro-2-methyl-phenoxy)-4-methyl-5-(trifluoromethyl)-3-pyridyl]-2-methyl-3-pyridyl]oxazolidin-2-one was purified by chiral SFC (ChiralPak AS column, 250×21.2 mm, 5 μm particle size, isocratic 80% CO₂/20% methanol (20 nM NH₃) over 8 minutes, flow rate=70 mL/min, column temperature=40° C.) to provide the separated, OBn-protected enantiomers.

Peak 1 (retention time 3.7 min) was isolated and debenzylated using standard hydrogenation conditions to provide 207.

Peak 2 (retention time 5.7 min) was isolated and debenzylated using standard hydrogenation conditions to provide 208.

### Example 3

### Compound 209

### 6-[2-(3,4-difluoro-2-methoxy-phenoxy)-5-fluoro-4-(trifluoromethyl)phenyl]-2-methyl-3-methylsulfinyl-1H-pyridin-4-one

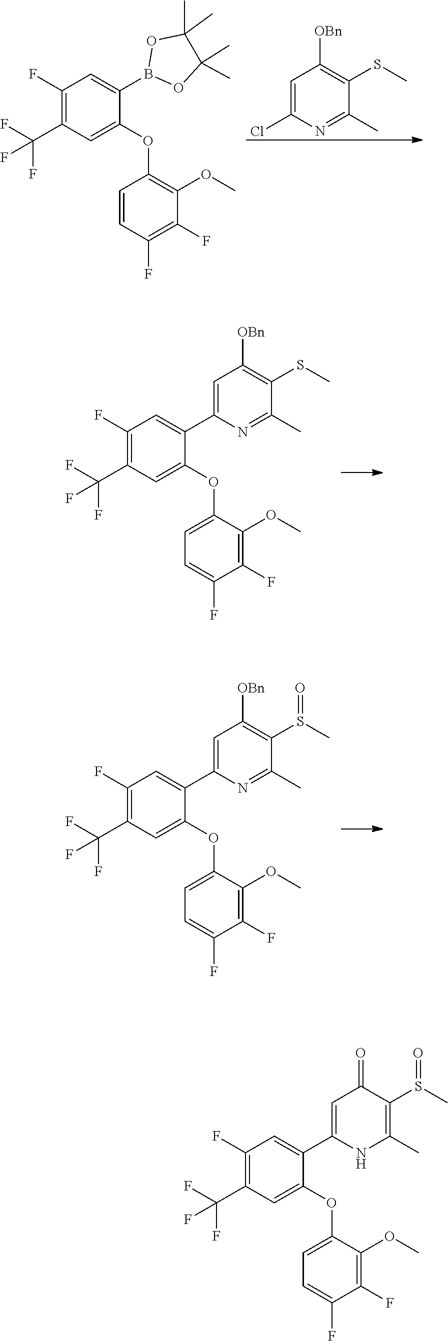 [SMILES: COc1c(Oc2cc(C(F)(F)F)c(F)cc2B2OC(C)(C)C(C)(C)O2)ccc(F)c1F ; CSc1c(OCc2ccccc2)cc(Cl)nc1C ; COc1c(Oc2cc(C(F)(F)F)c(F)cc2-c2cc(OCc3ccccc3)c(SC)c(C)n2)ccc(F)c1F ; COc1c(Oc2cc(C(F)(F)F)c(F)cc2-c2cc(OCc3ccccc3)c(S(C)=O)c(C)n2)ccc(F)c1F ; COc1c(Oc2cc(C(F)(F)F)c(F)cc2-c2cc(=O)c(S(C)=O)c(C)[nH]2)ccc(F)c1F]

### Step 1: 4-benzyloxy-6-[2-(3,4-difluoro-2-methoxy-phenoxy)-5-fluoro-4-(trifluoromethyl)phenyl]-2-methyl-3-methylsulfanyl-pyridine

2-[2-(3,4-Difluoro-2-methoxy-phenoxy)-5-fluoro-4-(trifluoromethyl)phenyl]-4,4,5,5-tetramethyl-1,3,2-dioxaborolane (961 mg, 1.07 mmol), 4-benzyloxy-6-chloro-2-methyl-3-methylsulfanyl-pyridine (150 mg, 0.536 mmol), Pd(dppf)Cl₂.DCM (88 mg, 0.11 mmol) and potassium carbonate (223 mg, 1.61 mmol) were suspended in 1,4-dioxane (3.0 mL) and water (750 μL) and nitrogen bubbled through the mixture for 5 min. The mixture was heated at 90° C. for 1 h, then cooled to room temperature, poured over water (20 mL) and diluted with TBME (20 mL). The organic layer was separated and the aqueous layer was extracted with TBME (2×20 mL). The combined organic extracts were washed with brine (20 mL), dried over magnesium sulfate and concentrated in vacuo. Purification by silica gel chromatography (80 g silica, 0-30% ethyl acetate/heptane provided 4-benzyloxy-6-[2-(3,4-difluoro-2-methoxy-phenoxy)-5-fluoro-4-(trifluoromethyl)phenyl]-2-methyl-3-methylsulfanyl-pyridine (200 mg, 66%) as a yellow oil. ESI-MS m/z calc. 565.12, found 566.7 (M+1)⁺. ¹H NMR (400 MHz, CDCl₃) δ 7.90 (d, J=11.1 Hz, 1H), 7.50 (s, 1H), 7.38-7.34 (m, 2H), 7.31-7.25 (m, 3H), 7.00 (d, J=5.8 Hz, 1H), 6.86-6.79 (m, 1H), 6.61-6.55 (m, 1H), 5.21 (s, 2H), 3.83 (d, J=1.8 Hz, 3H), 2.77 (s, 3H), 2.38 (s, 3H). ¹⁹F NMR (376 MHz, CDCl₃) δ −61.39, −120.58, −139.43, −151.11 ppm.

### Step 2: 4-benzyloxy-6-[2-(3,4-difluoro-2-methoxy-phenoxy)-5-fluoro-4-(trifluoromethyl)phenyl]-2-methyl-3-methylsulfinyl-pyridine

4-Benzyloxy-6-[2-(3,4-difluoro-2-methoxy-phenoxy)-5-fluoro-4-(trifluoromethyl)phenyl]-2-methyl-3-methylsulfanyl-pyridine (47.5 mg, 0.0840 mmol) in dichloromethane (5 mL) was cooled to −40° C. then treated with mCPBA (31.8 mg of 60% w/v, 0.111 mmol). The mixture was stirred for 20 min then washed with aqueous sodium bicarbonate. The organic layer was dried over magnesium sulfate, filtered and concentrated in vacuo to provided 4-benzyloxy-6-[2-(3,4-difluoro-2-methoxy-phenoxy)-5-fluoro-4-(trifluoromethyl)phenyl]-2-methyl-3-methylsulfinyl-pyridine (46.2 mg, 95%). ESI-MS m/z calc. 581.11, found 582.3 (M+1)⁺.

### Step 3: 6-[2-(3,4-difluoro-2-methoxy-phenoxy)-5-fluoro-4-(trifluoromethyl)phenyl]-2-methyl-3-methylsulfinyl-1H-pyridin-4-one, Compound 209

A solution of 4-benzyloxy-6-[2-(3,4-difluoro-2-methoxy-phenoxy)-5-fluoro-4-(trifluoromethyl)phenyl]-2-methyl-3-methylsulfinyl-pyridine (46.2 mg, 0.0795 mmol) in methanol (3 mL) was stirred with 10% Pd/C (17 mg, 0.016 mmol) under hydrogen atmosphere for 1 h. Purification by reverse phase HPLC (C18, acetonitrile/0.1% ammonium hydroxide gradient) provided 6-[2-(3,4-difluoro-2-methoxy-phenoxy)-5-fluoro-4-(trifluoromethyl)phenyl]-2-methyl-3-methylsulfinyl-1H-pyridin-4-one (13 mg, 33%). ¹H NMR (500 MHz, DMSO-d₆) δ 11.92 (s, 1H), 7.93 (d, J=10.8 Hz, 1H), 7.26 (d, J=5.8 Hz, 1H), 7.20 (td, J=9.8, 8.5 Hz, 1H), 6.99 (ddd, J=9.4, 5.0, 2.2 Hz, 1H), 6.50 (s, 1H), 3.82 (d, J=1.2 Hz, 3H), 2.92 (s, 3H), 2.61 (s, 3H). ESI-MS m/z calc. 491.06, found 492.2 (M+1)⁺.

### Compound 210

### 6-[2-(3,4-difluoro-2-methoxy-phenoxy)-5-fluoro-4-(trifluoromethyl)phenyl]-N,2-dimethyl-4-oxo-1,4-dihydropyridine-3-sulfonamide

### Step 1: N-benzyl-6-[2-(3,4-difluoro-2-methoxy-phenoxy)-5-fluoro-4-(trifluoromethyl)phenyl]-4-hydroxy-N,2-dimethyl-pyridine-3-sulfonamide

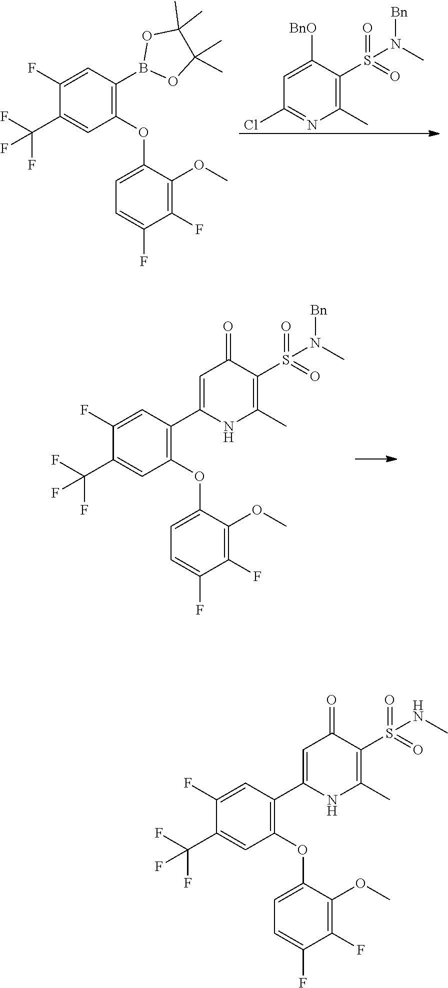 [SMILES: COc1c(Oc2cc(C(F)(F)F)c(F)cc2B2OC(C)(C)C(C)(C)O2)ccc(F)c1F ; Cc1nc(Cl)cc(OCc2ccccc2)c1S(=O)(=O)N(C)Cc1ccccc1 ; COc1c(Oc2cc(C(F)(F)F)c(F)cc2-c2cc(=O)c(S(=O)(=O)N(C)Cc3ccccc3)c(C)[nH]2)ccc(F)c1F ; CNS(=O)(=O)c1c(C)[nH]c(-c2cc(F)c(C(F)(F)F)cc2Oc2ccc(F)c(F)c2OC)cc1=O]

A mixture of 2-[2-(3,4-difluoro-2-methoxy-phenoxy)-5-fluoro-4-(trifluoromethyl)phenyl]-4,4,5,5-tetramethyl-1,3,2-dioxaborolane (150 mg, 0.231 mmol), N-benzyl-4-benzyloxy-6-chloro-N,2-dimethyl-pyridine-3-sulfonamide (50 mg, 0.12 mmol) and potassium carbonate (42 mg, 0.30 mmol) in 1,4-dioxane (1.5 mL) and water (0.3 mL) was degassed by bubbling through argon for 5 min. XPhos Pd G3 (10 mg, 0.012 mmol) was added and the reaction mixture was heated at 100° C. for 2 h. The reaction mixture was cooled to room temperature and partitioned between water and ethyl acetate. The aqueous phase was extracted with ethyl acetate and the combined organic were washed with brine, dried over magnesium sulfate, filtered and concentrated under reduced pressure. Purification by silica gel chromatography (0-30% ethyl acetate/heptane) provided N-benzyl-6-[2-(3,4-difluoro-2-methoxy-phenoxy)-5-fluoro-4-(trifluoromethyl)phenyl]-4-hydroxy-N,2-dimethyl-pyridine-3-sulfonamide (30 mg, 41%) as a white solid. ¹H NMR (400 MHz, CD₃OD) δ 7.68 (d, J=10.1 Hz, 1H), 7.35-7.29 (m, 4H), 7.26-7.21 (m, 2H), 7.00 (q, J=9.2 Hz, 1H), 6.85 (qd, J=4.7, 2.3 Hz, 1H), 6.55 (s, 1H), 4.41 (s, 2H), 3.85 (d, J=1.8 Hz, 3H), 2.73 (s, 3H), 2.72 (s, 3H). ¹⁹F NMR (376 MHz, CD₃OD) δ −63.1 (d, J=12.9 Hz, 3F), −122.1 (s, 1F), −141.2-−141.2 (m, 1F), −153.5 (dd, J=19.2, 7.6 Hz, 1F). ESI-MS m/z calc. 612.12, found 613.05 (M+1)⁺.

### Step 2: 6-[2-(3,4-difluoro-2-methoxy-phenoxy)-5-fluoro-4-(trifluoromethyl)phenyl]-4-hydroxy-N,2-dimethyl-pyridine-3-sulfonamide, Compound 210

A solution of N-benzyl-6-[2-(3,4-difluoro-2-methoxy-phenoxy)-5-fluoro-4-(trifluoromethyl)phenyl]-4-hydroxy-N,2-dimethyl-pyridine-3-sulfonamide (20 mg, 0.032 mmol) in sulfuric acid (0.2 mL, 3.8 mmol) was stirred at room temperature for 1 h and then water was added. The mixture was extracted with ethyl acetate (2×), and the combined extracts washed with brine, dried over magnesium sulfate, filtered and concentrated under reduced pressure. Purification by reverse phase chromatography (C18, 30-80% acetonitrile/water, each with 0.1% formic acid) provided 6-[2-(3,4-difluoro-2-methoxy-phenoxy)-5-fluoro-4-(trifluoromethyl)phenyl]-4-hydroxy-N,2-dimethyl-pyridine-3-sulfonamide (10 mg, 60%) as a white solid. ESI-MS m/z calc. 522.07, found 522.98 (M+1)⁺. ¹H NMR (400 MHz, CD₃OD) δ 7.68 (d, J=10.1 Hz, 1H), 7.21 (d, J=5.5 Hz, 1H), 7.02-6.95 (m, 1H), 6.84 (qd, J=4.7, 2.3 Hz, 1H), 6.61 (s, 1H), 3.84 (d, J=1.8 Hz, 3H), 2.71 (s, 3H), 2.50 (s, 3H). ¹⁹F NMR (376 MHz, CD₃OD) δ 63.2 (d, J=13.3 Hz, 3F), δ122.1 (d, J=4.2 Hz, 1F), δ141.3 (s, 1F), δ153.6 (dd, J=19.1, 7.4 Hz, 1F).

### Compound 211

### 6-[2-(3,4-difluoro-2-methoxy-phenoxy)-5-fluoro-4-(trifluoromethyl)phenyl]-2-methyl-4-oxo-1H-pyridine-3-sulfonamide

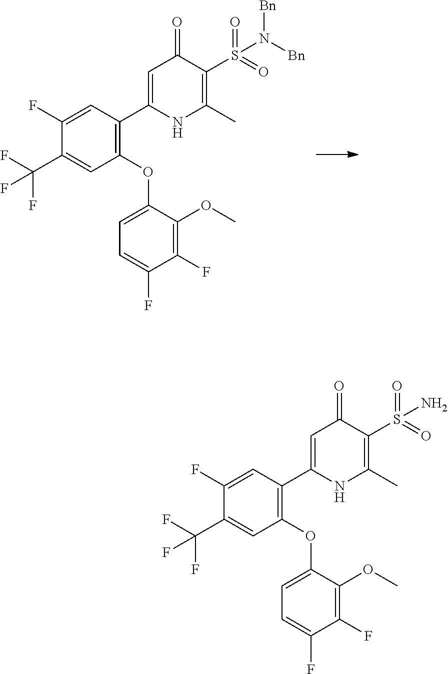 [SMILES: COc1c(Oc2cc(C(F)(F)F)c(F)cc2-c2cc(=O)c(S(=O)(=O)N(Cc3ccccc3)Cc3ccccc3)c(C)[nH]2)ccc(F)c1F ; COc1c(Oc2cc(C(F)(F)F)c(F)cc2-c2cc(=O)c(S(N)(=O)=O)c(C)[nH]2)ccc(F)c1F]

6-[2-(3,4-Difluoro-2-methoxy-phenoxy)-5-fluoro-4-(trifluoromethyl)phenyl]-2-methyl-4-oxo-1H-pyridine-3-sulfonamide was prepared from using a procedure analogous to that found in Compound 210. Step 1 was performed using N,N-dibenzyl-4-benzyloxy-6-chloro-2-methyl-pyridine-3-sulfonamide (Intermediate A-38). ¹H NMR (400 MHz, CD₃OD) δ 7.68 (d, J=10.1 Hz, 1H), 7.13 (d, J=5.5 Hz, 1H), 7.05-6.98 (m, 1H), 6.88 (qd, J=4.7, 2.4 Hz, 1H), 6.67 (s, 1H), 3.82 (d, J=1.8 Hz, 3H), 2.73 (s, 3H). ¹⁹F NMR (376 MHz, CD₃OD) δ −63.2 (d, J=13.3 Hz, 3F), −122.4 (s, 1F), −140.9 (d, J=12.3 Hz, 1F), −153.4 (dd, J=19.1, 7.4 Hz, 1F). ESI-MS m/z calc. 508.05, found 509.07 (M+1)⁺.

### Compound 212

### ethyl 2-[2-(3,4-difluoro-2-methoxy-phenoxy)-5-fluoro-4-(trifluoromethyl)phenyl]-6-methyl-5-(methylsulfonimidoyl)-4-oxo-1H-pyridine-3-carboxylate

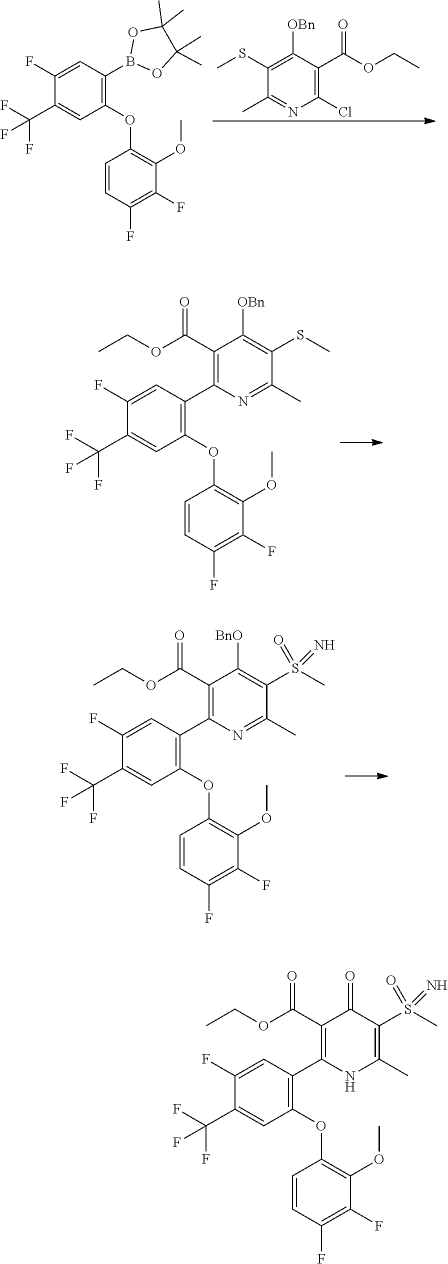

### Step 1: ethyl 4-benzyloxy-2-[2-(3,4-difluoro-2-methoxy-phenoxy)-5-fluoro-4-(trifluoromethyl)phenyl]-6-methyl-5-methylsulfanyl-pyridine-3-carboxylate

Ethyl 4-benzyloxy-2-[2-(3,4-difluoro-2-methoxy-phenoxy)-5-fluoro-4-(trifluoromethyl)phenyl]-6-methyl-5-methylsulfanyl-pyridine-3-carboxylate was prepared from 2-[2-(3,4-difluoro-2-methoxy-phenoxy)-5-fluoro-4-(trifluoromethyl)phenyl]-4,4,5,5-tetramethyl-1,3,2-dioxaborolane and ethyl 4-benzyloxy-2-chloro-6-methyl-5-methylsulfanyl-pyridine-3-carboxylate using a cross-coupling procedure analogous to that found in Compound 209, step 1. ESI-MS m/z calc. 637.14, found 638.4 (M+1)⁺.

### Step 2: ethyl 4-benzyloxy-2-[2-(3,4-difluoro-2-methoxy-phenoxy)-5-fluoro-4-(trifluoromethyl)phenyl]-6-methyl-5-(methylsulfonimidoyl)pyridine-3-carboxylate

Ethyl 4-benzyloxy-2-[2-(3,4-difluoro-2-methoxy-phenoxy)-5-fluoro-4-(trifluoromethyl)phenyl]-6-methyl-5-(methylsulfonimidoyl)pyridine-3-carboxylate was prepared from ethyl 4-benzyloxy-2-[2-(3,4-difluoro-2-methoxy-phenoxy)-5-fluoro-4-(trifluoromethyl)phenyl]-6-methyl-5-methylsulfanyl-pyridine-3-carboxylate using a procedure analogous to that found in Intermediate A-24. ESI-MS m/z calc. 668.14, found 669.0 (M+1)⁺.

### Step 3: ethyl 2-[2-(3,4-difluoro-2-methoxy-phenoxy)-5-fluoro-4-(trifluoromethyl)phenyl]-6-methyl-5-(methylsulfonimidoyl)-4-oxo-1H-pyridine-3-carboxylate, Compound 212

Ethyl 2-[2-(3,4-difluoro-2-methoxy-phenoxy)-5-fluoro-4-(trifluoromethyl)phenyl]-6-methyl-5-(methylsulfonimidoyl)-4-oxo-1H-pyridine-3-carboxylate was prepared using a debenzylation procedure analogous to that found in Compound 209, step 3. ¹H NMR (400 MHz, DMSO-d₆) δ 7.66 (d, J=10.2 Hz, 1H), 7.29-7.12 (m, 2H), 6.88 (ddd, J=9.3, 5.1, 2.2 Hz, 1H), 4.02 (q, J=7.1 Hz, 2H), 3.82 (d, J=1.1 Hz, 3H), 3.38 (s, 8H), 2.61 (s, 3H), 0.93 (t, J=7.1 Hz, 3H). ESI-MS m/z calc. 578.10, found 579.3 (M+1)⁺.

### Compound 213

### 2-[2-(3,4-difluoro-2-methoxy-phenoxy)-5-fluoro-4-(trifluoromethyl)phenyl]-6-methyl-3-(methylsulfonimidoyl)-1H-pyridin-4-one

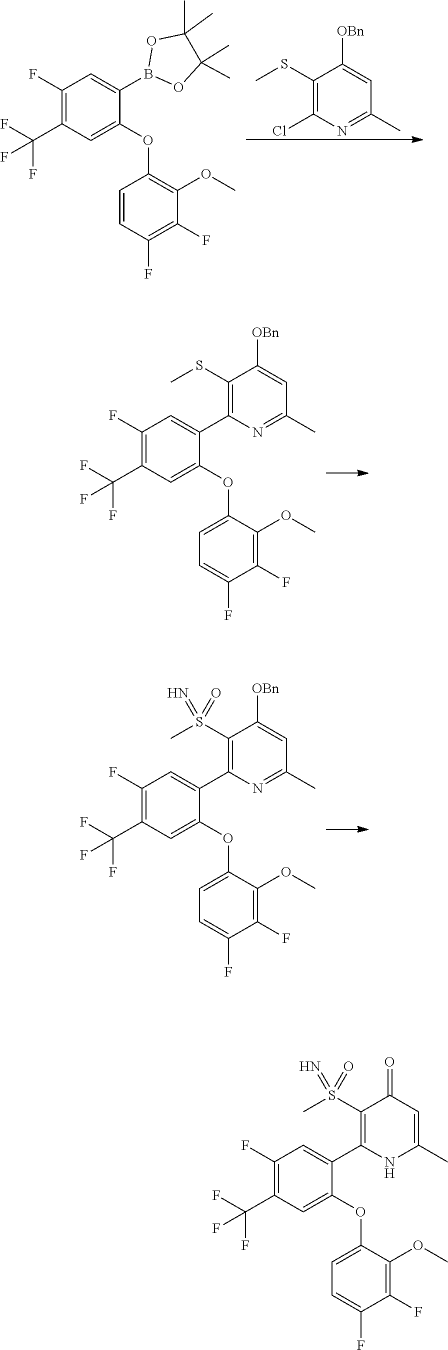

### Step 1: 4-benzyloxy-2-[2-(3,4-difluoro-2-methoxy-phenoxy)-5-fluoro-4-(trifluoromethyl)phenyl]-6-methyl-3-methylsulfanyl-pyridine

4-Benzyloxy-2-[2-(3,4-difluoro-2-methoxy-phenoxy)-5-fluoro-4-(trifluoromethyl)phenyl]-6-methyl-3-methylsulfanyl-pyridine was prepared from 2-[2-(3,4-difluoro-2-methoxy-phenoxy)-5-fluoro-4-(trifluoromethyl)phenyl]-4,4,5,5-tetramethyl-1,3,2-dioxaborolane and 4-benzyloxy-2-chloro-6-methyl-3-methylsulfanyl-pyridine using a cross-coupling procedure analogous to that found in Compound 209, step 1. ESI-MS m/z calc. 565.12, found 566.7 (M+1)⁺.

### Step 2: 2-[2-(3,4-difluoro-2-methoxy-phenoxy)-5-fluoro-4-(trifluoromethyl)phenyl]-6-methyl-3-(methylsulfonimidoyl)-1H-pyridin-4-one, Compound 213

[4-Benzyloxy-2-[2-(3,4-difluoro-2-methoxy-phenoxy)-5-fluoro-4-(trifluoromethyl)phenyl]-6-methyl-3-pyridyl]-imino-methyl-oxo-6-sulfane was prepared from 4-benzyloxy-2-[2-(3,4-difluoro-2-methoxy-phenoxy)-5-fluoro-4-(trifluoromethyl)phenyl]-6-methyl-3-methylsulfanyl-pyridine using a procedure analogous to that found in Intermediate A-24. ESI-MS m/z calc. 596.54, found 597.7 (M+1)⁺.

The benzyl-protected intermediate was deprotected using a procedure analogous to that found in Compound 209, step 3 using ethyl acetate as the solvent to provide 2-[2-(3,4-difluoro-2-methoxy-phenoxy)-5-fluoro-4-(trifluoromethyl)phenyl]-6-methyl-3-(methylsulfonimidoyl)-1H-pyridin-4-one (Compound 213). ESI-MS m/z calc. 506.07, found 507.6 (M+1)⁺; 505.5 (M−1)⁻. ¹H NMR (400 MHz, CD₃OD) δ 7.61-7.57 (m, 1H), 7.08-6.89 (m, 3H), 6.45-6.44 (m, 1H), 3.90-3.87 (m, 3H), 3.30-3.26 (m, 3H), 2.35-2.33 (m, 3H). ¹⁹F NMR (376 MHz, DMSO-d₆) δ −60.07, −123.44, −140.32, −152.55.

### Example 4

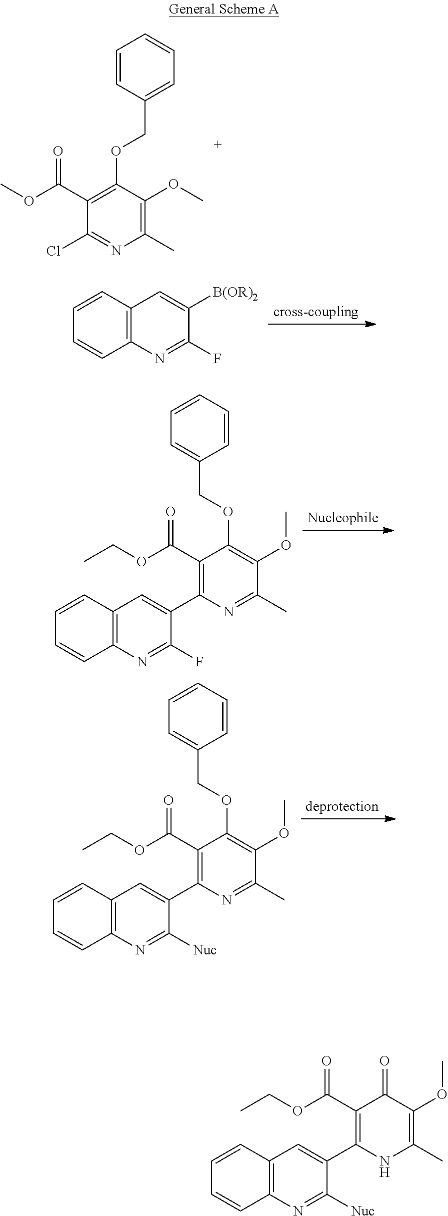

### Preparation of Amine Nucleophiles

### Amine 1: (3S,4S)-3-methoxy-4-methyl-pyrrolidine

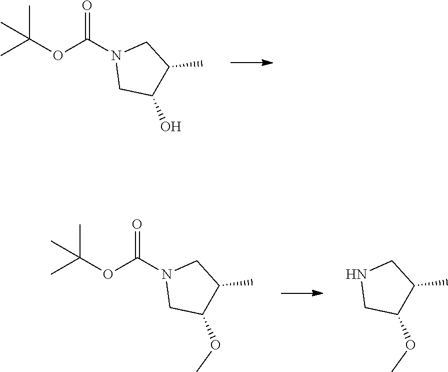

### Step 1: tert-butyl (3S,4S)-3-methoxy-4-methyl-pyrrolidine-1-carboxylate

To solution of tert-butyl (3S,4S)-3-hydroxy-4-methyl-pyrrolidine-1-carboxylate (3.0 g, 15 mmol) in THF (6 mL) cooled on an ice-bath was added sodium hydride (720 mg, 60% dispersion in mineral oil, 30.0 mmol) and the mixture stirred for 20 min followed by the addition of iodomethane (1.8 mL, 29 mmol). The temperature was allowed to rise to room temperature and the mixture stirred for a further 30 min. The reaction mixture was partitioned between ethyl acetate (10 mL) and water (10 mL). The aqueous layer was further extracted with ethyl acetate (10 mL). The organic extracts were washed with brine (20 mL), dried over magnesium sulfate, filtered and concentrated. Purification using silica gel chromatography (0-100% ethyl acetate/petroleum ether) provided tert-butyl (3S,4S)-3-methoxy-4-methyl-pyrrolidine-1-carboxylate (2.3 g, 72%). ESI-MS m/z calc. 215.15, found 216.4 (M+1)⁺. ¹H NMR (400 MHz, CDCl₃) δ 3.67 (td, J=4.5, 2.2 Hz, 1H), 3.49 (td, J=10.3, 9.8, 4.8 Hz, 2H), 3.34 (s, 4H), 3.02 (t, J=9.9 Hz, 1H), 2.39-2.11 (m, 1H), 1.40 (s, 9H), 1.05 (d, J=6.9 Hz, 3H).

### Step 2: (3S,4S)-3-methoxy-4-methyl-pyrrolidine

To a solution of tert-butyl (3S,4S)-3-methoxy-4-methyl-pyrrolidine-1-carboxylate (3.0 g, 13.9 mmol) in 1,4-dioxane (4 mL) was added hydrogen chloride (10.4 mL of 4 M, 42 mmol) and the mixture stirred at room temperature for 2 h. The solution was concentrated in vacuo to provide (3S,4S)-3-methoxy-4-methyl-pyrrolidine hydrochloride hydrate (2.1 g, 89%). ¹H NMR (400 MHz, CDCl₃) δ 3.78 (ddd, J=4.9, 3.9, 1.2 Hz, 1H), 3.59-3.38 (m, 2H), 3.36 (s, 4H), 3.01 (tt, J=11.2, 7.1 Hz, 1H), 2.36 (dtd, J=11.5, 7.1, 4.3 Hz, 1H), 1.11 (d, J=6.8 Hz, 3H).

### Amine 2: 7,7-difluoro-1,2,3,3a,4,5,6,7a-octahydroisoindole

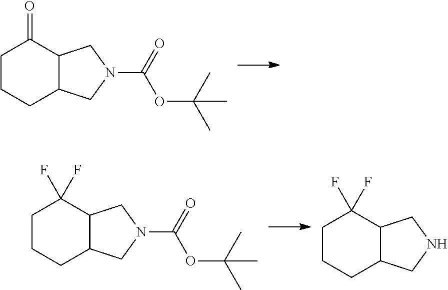 [SMILES: CC(C)(C)OC(=O)N1CC2CCCC(=O)C2C1 ; CC(C)(C)OC(=O)N1CC2CCCC(F)(F)C2C1 ; FC1(F)CCCC2CNCC21]

### Step 1: tert-butyl 7,7-difluoro-3,3a,4,5,6,7a-hexahydro-1H-isoindole-2-carboxylate

A solution of tert-butyl 7-oxo-3,3a,4,5,6,7a-hexahydro-1H-isoindole-2-carboxylate (300 mg, 1.25 mmol) in DCE (4.0 mL) was slowly added to a solution of Deoxo-Fluor® (411 mg, 1.86 mmol) in DCE (4.0 mL) and the mixture stirred overnight at 80° C. The mixture was cooled to 0° C. and saturated aqueous sodium bicarbonate was slowly added. After stirring for 20 min, the reaction was extracted with DCM (3×). The combined extracts were dried over sodium sulfate, filtered and concentrated. Purification by silica gel chromatography (24 g silica, 0-100% ethyl acetate/hexanes) provided tert-butyl 7,7-difluoro-3,3a,4,5,6,7a-hexahydro-1H-isoindole-2-carboxylate. ESI-MS m/z calc. 261.15, found 261.962 (M+1)⁺.

### Step 2: 7,7-difluoro-1,2,3,3a,4,5,6,7a-octahydroisoindole

tert-Butyl 7,7-difluoro-3,3a,4,5,6,7a-hexahydro-1H-isoindole-2-carboxylate (1.25 g, 3.83 mmol) was dissolved in a solution of HCl in dioxane (10 mL of 4 M, 40 mmol) at room temperature and stirred for 64 h. The reaction was concentrated to provide 7,7-difluoro-1,2,3,3a,4,5,6,7a-octahydroisoindole hydrochloride (1.08 g, 100%) as a yellow solid. ¹H NMR (301 MHz, CDCl₃) δ 9.92 (br s, 2H), 3.50-3.05 (m, 4H), 2.83-2.64 (m, 2H), 1.96-1.85 (m, 2H), 1.81-1.63 (m, 4H). ¹⁹F NMR (283 MHz, CDCl₃) δ −90.8-−91.9 (m, 1F), −96.4-−97.3 (m, 1F).

### Compound 214

### ethyl 5-methoxy-6-methyl-2-[2-[(3R)-3-methyl-1-piperidyl]-3-quinolyl]-4-oxo-1H-pyridine-3-carboxylate

### Step 1: ethyl 4-benzyloxy-2-(2-fluoro-3-quinolyl)-5-methoxy-6-methyl-pyridine-3-carboxylate

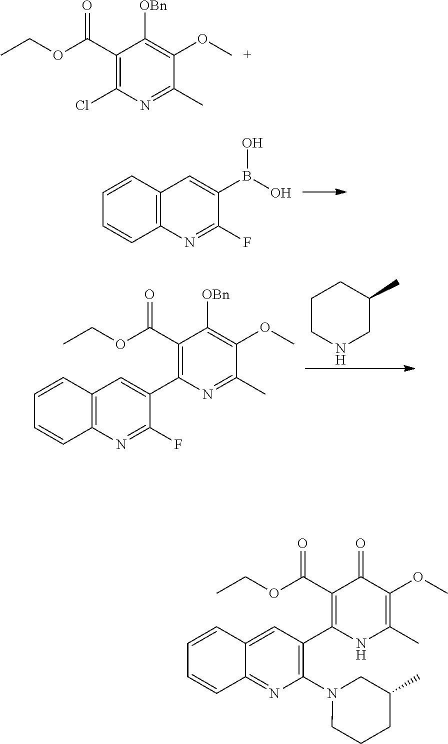

A mixture of ethyl 4-benzyloxy-2-chloro-5-methoxy-6-methyl-pyridine-3-carboxylate (300 mg, 0.893 mmol), (2-fluoro-3-quinolyl)boronic acid (255 mg, 1.34 mmol), potassium phosphate (475 mg, 2.24 mmol), SPhos Pd G3 (141 mg, 0.181 mmol) in 1,4-dioxane (4.5 mL) and water (1.5 mL) was degassed using nitrogen bubbling for 1 minute. The mixture was heated in a sealed vial under nitrogen at 70° C. for 18 h. The reaction mixture was diluted with ethyl acetate (5 mL) and water (5 mL) and the layers separated. The aqueous layer was extracted with additional ethyl acetate (2×), and the combined organic layers dried over sodium sulfate, filtered and concentrated. Purification using silica gel chromatography (0-25% hexane/ethyl acetate) provided ethyl 4-benzyloxy-2-(2-fluoro-3-quinolyl)-5-methoxy-6-methyl-pyridine-3-carboxylate (111.0 mg, 27%). ¹H NMR (400 MHz, CD₃OD) δ 8.49-8.42 (m, 1H), 8.02 (dd, J=8.2, 1.4 Hz, 1H), 7.94-7.88 (m, 1H), 7.86-7.79 (m, 1H), 7.64 (ddd, J=8.2, 6.9, 1.3 Hz, 1H), 7.47-7.41 (m, 2H), 7.39-7.29 (m, 3H), 5.30 (s, 2H), 4.02 (q, J=7.1 Hz, 2H), 3.95 (s, 3H), 2.58 (s, 3H), 0.87 (t, J=7.1 Hz, 3H). ESI-MS m/z calc. 446.16, found 447.0 (M+1)⁺.

### Step 2: ethyl 5-methoxy-6-methyl-2-[2-[(3R)-3-methyl-1-piperidyl]-3-quinolyl]-4-oxo-1H-pyridine-3-carboxylate, Compound 214

Ethyl 4-benzyloxy-2-(2-fluoro-3-quinolyl)-5-methoxy-6-methyl-pyridine-3-carboxylate (20 mg, 0.043 mmol), (3R)-3-methylpiperidine (4.2 mg, 0.043 mmol), cesium carbonate (23 mg, 0.071 mmol), and NMP (0.5 mL) were stirred at 80° C. for 1 h. The mixture was filtered and purified by reverse phase chromatography (C18, 10-70% acetonitrile/5 mM HCl over 15 min) to provide the benzyl-protected intermediate. The benzyl-protected intermediate was stirred in methanol (2 mL) with 10% Pd/C (5 mg) under hydrogen atmosphere for 2 h, then filtered and purified by reverse phase chromatography (C18, 10-60% acetonitrile/5 mM HCl over 15 min) to provide ethyl 5-methoxy-6-methyl-2-[2-[(3R)-3-methyl-1-piperidyl]-3-quinolyl]-4-oxo-TH-pyridine-3-carboxylate (214, 12.8 mg, 680). ESI-MS m/z calc. 435.22, found 436.0 (M+1)⁺.

The following compounds were synthesized using the conditions analogous to those described for Compound 214, step 2 using either commercially available amines, the amines described above, or phenols as the nucleophile.

| Cmpd | | MW & Found |
|---|---|---|
| No. | Compound Name | [M + H]⁺ |
| 215 | ethyl 2-[2-(8-azaspiro[4.5]decan-8-yl)-3-quinolyl]-5- | 475.58; 476.4 |
| | methoxy-6-methyl-4-oxo-1H-pyridine-3-carboxylate | |
| 216 | ethyl 2-[2-(7-azaspiro[3.5]nonan-7-yl)-3-quinolyl]-5- | 461.55; 462.3 |
| | methoxy-6-methyl-4-oxo-1H-pyridine-3-carboxylate | |
| 217 | ethyl 2-[2-(3-azabicyclo[3.2.1]octan-3-yl)-3-quinolyl]-5- | 447.53; 448.3 |
| | methoxy-6-methyl-4-oxo-1H-pyridine-3-carboxylate | |
| 218 | ethyl 2-[2-(4-fluoro-4-methyl-1-piperidyl)-3-quinolyl]-5- | 453.51; 454.3 |
| | methoxy-6-methyl-4-oxo-1H-pyridine-3-carboxylate | |
| 219 | ethyl 2-[2-[(3S,5R)-3,5-dimethyl-1-piperidyl]-3-quinolyl]- | 449.54; 450.3 |
| | 5-methoxy-6-methyl-4-oxo-1H-pyridine-3-carboxylate | |
| 220 | ethyl 2-[2-(4-isopropyl-1-piperidyl)-3-quinolyl]-5- | 463.57; 464.4 |
| | methoxy-6-methyl-4-oxo-1H-pyridine-3-carboxylate | |
| 221 | ethyl 5-methoxy-6-methyl-2-[2-[(3S)-3-methyl-1- | 435.52; 436.3 |
| | piperidyl]-3-quinolyl]-4-oxo-1H-pyridine-3-carboxylate | |
| 222 | ethyl 5-methoxy-6-methyl-2-[2-(4-methyl-1-piperidyl)-3- | 435.52; 436.3 |
| | quinolyl]-4-oxo-1H-pyridine-3-carboxylate | |
| 223 | ethyl 2-[2-(3,4-difluoro-2-methoxy-phenoxy)-3-quinolyl]- | 496.46; 497.3 |
| | 5-methoxy-6-methyl-4-oxo-1H-pyridine-3-carboxylate | |
| 224 | ethyl 5-methoxy-2-[2-[(3R)-3-methoxy-1-piperidyl]-3- | 451.51; 452.2 |
| | quinolyl]-6-methyl-4-oxo-1H-pyridine-3-carboxylate | |
| 225 | ethyl 2-[2-(2-chloro-4-fluoro-phenoxy)-3-quinolyl]-5- | 482.89; 483.2 |
| | methoxy-6-methyl-4-oxo-1H-pyridine-3-carboxylate | |
| 226 | ethyl 2-[2-(3,3-difluoro-1-piperidyl)-3-quinolyl]-5- | 457.47; 458.3 |
| | methoxy-6-methyl-4-oxo-1H-pyridine-3-carboxylate | |
| 227 | ethyl 5-methoxy-6-methyl-2-[2-(1,4-oxazepan-4-yl)-3- | 437.49; 438.3 |
| | quinolyl]-4-oxo-1H-pyridine-3-carboxylate | |
| 228 | ethyl 2-[2-[(3aS,6aS)-2,3,3a,4,6,6a-hexahydrofuro[2,3- | 449.5; 450.3 |
| | c]pyrrol-5-yl]-3-quinolyl]-5-methoxy-6-methyl-4-oxo- | |
| | 1H-pyridine-3-carboxylate | |
| 229 | ethyl 5-methoxy-2-[2-(4-methoxy-1-piperidyl)-3- | 451.51; 452.3 |
| | quinolyl]-6-methyl-4-oxo-1H-pyridine-3-carboxylate | |
| 230 | ethyl 2-[2-(6-azaspiro[3.5]nonan-6-yl)-3-quinolyl]-5- | 461.55; 462.3 |
| | methoxy-6-methyl-4-oxo-1H-pyridine-3-carboxylate | |
| 231 | ethyl 5-methoxy-2-[2-[(3S)-3-methoxy-1-piperidyl]-3- | 451.51; 452.3 |
| | quinolyl]-6-methyl-4-oxo-1H-pyridine-3-carboxylate | |
| 232 | ethyl 5-methoxy-6-methyl-4-oxo-2-[2-[4- | 489.49; 490.3 |
| | (trifluoromethyl)-1-piperidyl]-3-quinolyl]-1H-pyridine-3- | |
| | carboxylate | |
| 233 | ethyl 2-[2-(azepan-1-yl)-3-quinolyl]-5-methoxy-6- | 435.52; 436.3 |
| | methyl-4-oxo-1H-pyridine-3-carboxylate | |
| 234 | ethyl 2-[2-(4,4-difluoroazepan-1-yl)-6-methyl-5- | 503.46; 504.7 |
| | (trifluoromethyl)-3-pyridyl]-5-methoxy-6-methyl-4-oxo- | |
| | 1H-pyridine-3-carboxylate | |
| 235 | ethyl 2-[2-[(3aR,6aS)-5,5-difluoro-1,3,3a,4,6,6a- | 483.51; 484.3 |
| | hexahydrocyclopenta[c]pyrrol-2-yl]-3-quinolyl]-5- | |
| | methoxy-6-methyl-4-oxo-1H-pyridine-3-carboxylate | |
| 236 | ethyl 2-[2-(6,6-difluoro-3-azabicyclo[3.1.0]hexan-3-yl)-3- | 455.45; 456.3 |
| | quinolyl]-5-methoxy-6-methyl-4-oxo-1H-pyridine-3- | |
| | carboxylate | |
| 237 | ethyl 2-[2-[(3aR,6aS)-3,3a,4,5,6,6a-hexahydro-1H- | 447.53; 448.3 |
| | cyclopenta[c]pyrrol-2-yl]-3-quinolyl]-5-methoxy-6- | |
| | methyl-4-oxo-1H-pyridine-3-carboxylate | |
| 238 | ethyl 2-[2-(3,3-dimethylpyrrolidin-1-yl)-3-quinolyl]-5- | 435.52; 436 |
| | methoxy-6-methyl-4-oxo-1H-pyridine-3-carboxylate | |
| 239 | ethyl 2-[2-[(3S,4R)-3,4-dimethylpyrrolidin-1-yl]-3- | 435.52; 436.3 |
| | quinolyl]-5-methoxy-6-methyl-4-oxo-1H-pyridine-3- | |
| | carboxylate | |
| 240 | ethyl 5-methoxy-6-methyl-4-oxo-2-[2-[3- | 475.46; 476.3 |
| | (trifluoromethyl)pyrrolidin-1-yl]-3-quinolyl]-1H- | |
| | pyridine-3-carboxylate | |
| 241 | ethyl 5-methoxy-2-[2-[3-methoxy-4- | 505.49; 506.3 |
| | (trifluoromethyl)pyrrolidin-1-yl]-3-quinolyl]-6-methyl-4- | |
| | oxo-1H-pyridine-3-carboxylate | |
| 242 | ethyl 2-[2-(3,3-difluoropyrrolidin-1-yl)-3-quinolyl]-5- | 443.44; 444.3 |
| | methoxy-6-methyl-4-oxo-1H-pyridine-3-carboxylate | |
| 243 | ethyl 5-methoxy-2-[2-[(3S,4S)-3-methoxy-4-methyl- | 451.51; 452.3 |
| | pyrrolidin-1-yl]-3-quinolyl]-6-methyl-4-oxo-1H-pyridine- | |
| | 3-carboxylate | |
| 244 | ethyl 5-methoxy-2-[2-[(3S)-3-methoxypyrrolidin-1-yl]-3- | 437.49; 438.3 |
| | quinolyl]-6-methyl-4-oxo-1H-pyridine-3-carboxylate | |
| 245 | ethyl 2-[2-(6-azaspiro[2.5]octan-6-yl)-3-quinolyl]-5- | 447.53; 448.3 |
| | methoxy-6-methyl-4-oxo-1H-pyridine-3-carboxylate | |
| 246 | ethyl 2-[2-(4,4-dimethyl-1-piperidyl)-3-quinolyl]-5- | 449.54; 450.3 |
| | methoxy-6-methyl-4-oxo-1H-pyridine-3-carboxylate | |
| 247 | ethyl 2-[2-(1,3,3a,4,5,6,7,7a-octahydroisoindol-2-yl)-3- | 461.55; 462.4 |
| | quinolyl]-5-methoxy-6-methyl-4-oxo-1H-pyridine-3- | |
| | carboxylate | |
| 248 | ethyl 2-[2-(7,7-difluoro-3,3a,4,5,6,7a-hexahydro-1H- | 497.53; 498.3 |
| | isoindol-2-yl)-3-quinolyl]-5-methoxy-6-methyl-4-oxo- | |
| | 1H-pyridine-3-carboxylate | |
| 249 | ethyl 5-methoxy-6-methyl-2-(2-morpholino-3-quinolyl)- | 423.46; 424.3 |
| | 4-oxo-1H-pyridine-3-carboxylate | |
| 250 | ethyl 5-methoxy-6-methyl-4-oxo-2-[2-(1-piperidyl)-3- | 421.49; 422.3 |
| | quinolyl]-1H-pyridine-3-carboxylate | |
| 251 | ethyl 5-methoxy-[2-[4,4-difluoro-1-piperidyl]-3- | 457.47; 458.2 |
| | quinolyl]-6-methyl-4-oxo-1H-pyridine-3-carboxylate | |

### Example 5

### Compound 252

### 2-[3-(4,4-difluoroazepan-1-yl)-6-(trifluoromethyl)-2-pyridyl]-4-methyl-1H-pyrimidin-6-one

### Step 1: 3-bromo-6-(trifluoromethyl)pyridine-2-carboxamide

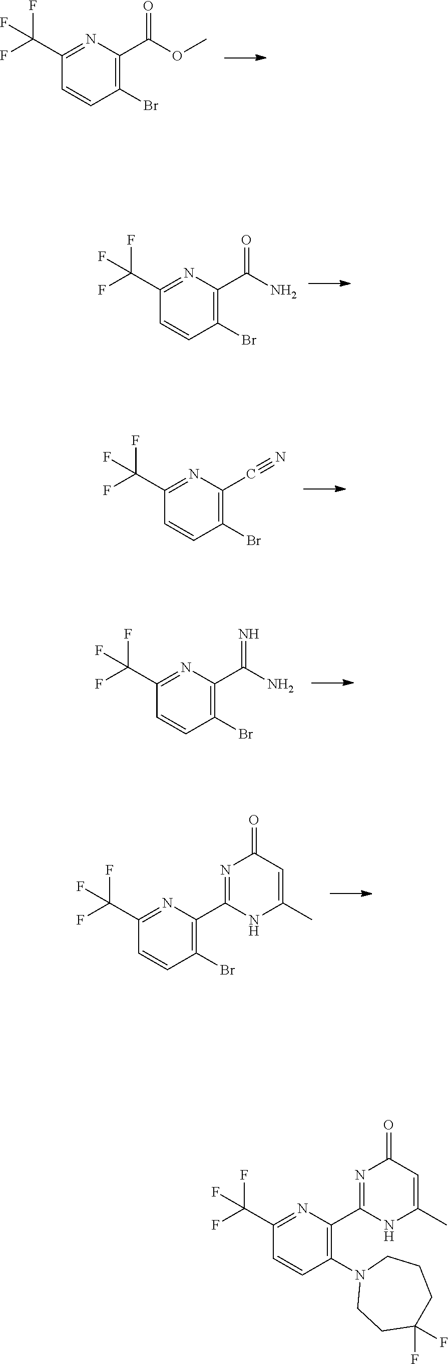 [SMILES: COC(=O)c1nc(C(F)(F)F)ccc1Br ; NC(=O)c1nc(C(F)(F)F)ccc1Br ; N#Cc1nc(C(F)(F)F)ccc1Br ; N=C(N)c1nc(C(F)(F)F)ccc1Br ; Cc1cc(=O)nc(-c2nc(C(F)(F)F)ccc2Br)[nH]1 ; Cc1cc(=O)nc(-c2nc(C(F)(F)F)ccc2N2CCCC(F)(F)CC2)[nH]1]

A mixture of methyl 3-bromo-6-(trifluoromethyl)pyridine-2-carboxylate (100 mg, 0.352 mmol) and ammonia in methanol (5 mL of 7 M, 35 mmol) was stirred at 60° C. for 18 h in a sealed tube. The reaction was concentrated under reduced pressure to afford 3-bromo-6-(trifluoromethyl)pyridine-2-carboxamide (91 mg, 96%) as a white solid. ¹H NMR (400 MHz, DMSO-d₆): 8.47 (d, J=8.7 Hz, 1H), 8.13 (br s, 1H), 7.96-7.88 (m, 2H). ESI-MS m/z calc. 267.95, found 269.0 (M+1)⁺.

### Step 2: 3-bromo-6-(trifluoromethyl)pyridine-2-carbonitrile

To a solution of 3-bromo-6-(trifluoromethyl)pyridine-2-carboxamide (91 mg, 0.34 mmol) in dichloromethane (5 mL) was added pyridine (0.13 mL, 1.6 mmol) then the mixture was cooled at 0° C. Trifluoroacetic anhydride (195 mg, 0.928 mmol) was added and the reaction was stirred at 0° C. for 1 h. The mixture was quenched with water and diluted with dichloromethane. The aqueous phase was extracted with additional dichloromethane (2×10 mL), and the combined organic extracts were washed with aqueous 5% citric acid solution and saturated aqueous sodium bicarbonate (20 mL). The organic phase was dried over sodium sulfate, filtered and concentrated under reduced pressure to afford 3-bromo-6-(trifluoromethyl)pyridine-2-carbonitrile (84 mg, 94%) as a white solid. ¹H NMR (400 MHz, DMSO-d₆): 8.72 (d, J=8.9 Hz, 1H), 8.21 (d, J=8.7 Hz, 1H). ¹⁹F NMR (377 MHz, DMSO-d₆): −66.57 (s, 3F).

### Step 3: 3-bromo-6-(trifluoromethyl)pyridine-2-carboxamidin

To a solution of 3-bromo-6-(trifluoromethyl)pyridine-2-carbonitrile (84 mg, 0.32 mmol) in methanol (3 mL) was added sodium methoxide (19 mg, 0.35 mmol) and the mixture stirred at room temperature for 18 h. Ammonium chloride (19 mg, 0.36 mmol) was added and the reaction was stirred at 80° C. for 5 h. The reaction was concentrated under reduced pressure to provid 3-bromo-6-(trifluoromethyl)pyridine-2-carboxamidine (95 mg, 65%) as a white solid. ESI-MS m/z cale. 266.96, found 268.0 (M+1)⁺.

### Step 4: 2-[3-bromo-6-(trifluoromethyl)-2-pyridyl]-4-methyl-1H-pyrimidin-6-one

To a solution of 3-bromo-6-(trifluoromethyl)pyridine-2-carboxamidine (50 mg, 0.099 mmol) and ethyl 3-oxobutanoate (36 mg, 35 μL, 0.28 mmol) in ethanol (1 mL) and water (0.1 mL) was added NaOH (8 mg, 0.2 mmol) and the reaction was stirred at room temperature for 18 h. The mixture was diluted with water (50 mL) and ethyl acetate (50 mL). The mixture was washed with 5% aqueous citric acid solution (10 mL) and saturated aqueous sodium bicarbonate (20 mL). The organic phase was dried over sodium sulfate, filtered and concentrated under reduced pressure. Purification by silica gel chromatography (0-100% ethyl acetate/heptane) provided 2-[3-bromo-6-(trifluoromethyl)-2-pyridyl]-4-methyl-1H-pyrimidin-6-one (10 mg, 30%) as a white solid. ¹H NMR (400 MHz, DMSO-d₆): 12.9 (br s, 1H), 8.61 (d, J=9.1 Hz, 1H), 8.05 (d, J=9.6 Hz, 1H), 6.37 (br s, 1H), 2.28 (s, 3H). ESI-MS m/z calc. 332.97, found 334.0 (M+1)⁺.

### Step 5: 2-[3-(4,4-difluoroazepan-1-yl)-6-(trifluoromethyl)-2-pyridyl]-4-methyl-1H-pyrimidin-6-one, Compound 252

A solution of 2-[3-bromo-6-(trifluoromethyl)-2-pyridyl]-4-methyl-1H-pyrimidin-6-one (30 mg, 0.085 mmol), 4,4-difluoroazepane hydrochloride (20 mg, 0.12 mmol), cesium carbonate (64 mg, 0.20 mmol) in toluene (3 mL) was bubbled with nitrogen for 5 min then rac-BINAP (9 mg, 0.015 mmol) and tris(dibenzylideneacetone)dipalladium (0) (10 mg, 0.011 mmol) were added. The mixture was bubbled with nitrogen for another 5 min then stirred at 100° C. for 18 h. Purification by silica gel column chromatography (0-50% ethyl acetate/heptane) provided 2-[3-(4,4-difluoroazepan-1-yl)-6-(trifluoromethyl)-2-pyridyl]-4-methyl-1H-pyrimidin-6-one (252, 7.83 mg, 23%) as a light yellow solid. ESI-MS m/z calc. 388.13, found 389.2 (M+1)⁺. ¹H NMR (400 MHz, CDCl₃) δ 10.43 (br. s., 1H), 7.56 (d, J=8.8 Hz, 1H), 7.41 (d, J=8.8 Hz, 1H), 6.21 (s, 1H), 3.49-3.40 (m, 2H), 3.35-3.27 (m, 2H), 2.33-2.17 (m, 5H), 2.14-2.00 (m, 2H), 1.95-1.83 (m, 2H).

### Compound 253

### 2-[2-[(3S,4S)-3-methoxy-4-methyl-pyrrolidin-1-yl]-3-quinolyl]-6-methyl-1H-pyridin-4-one

### Step 1: 3-(4-benzyloxy-6-methyl-2-pyridyl)-2-chloro-quinoline

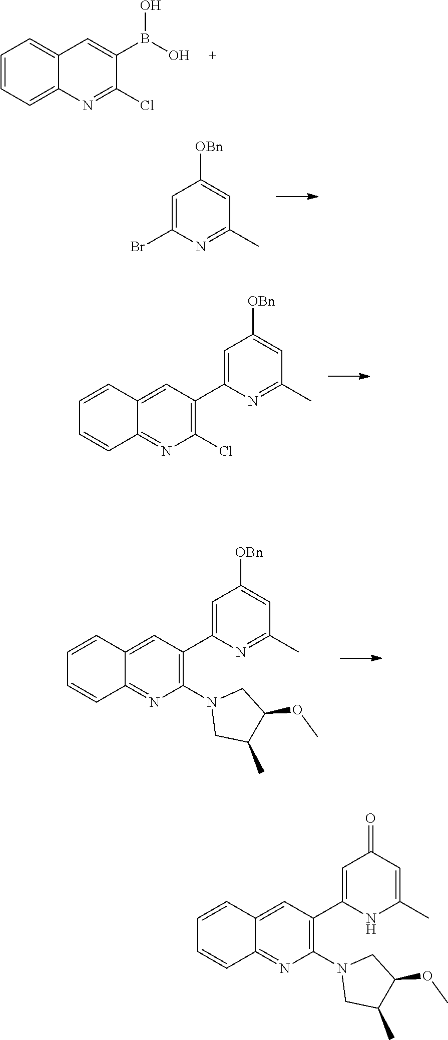 [SMILES: OB(O)c1cc2ccccc2nc1Cl ; Cc1cc(OCc2ccccc2)cc(Br)n1 ; Cc1cc(OCc2ccccc2)cc(-c2cc3ccccc3nc2Cl)n1 ; CO[C@@H]1CN(c2nc3ccccc3cc2-c2cc(OCc3ccccc3)cc(C)n2)C[C@@H]1C ; CO[C@@H]1CN(c2nc3ccccc3cc2-c2cc(=O)cc(C)[nH]2)C[C@@H]1C]

3-(4-Benzyloxy-6-methyl-2-pyridyl)-2-chloro-quinoline was prepared from (2-chloro-3-quinolyl)boronic acid and 4-benzyloxy-2-bromo-6-methyl-pyridine using a cross-coupling procedure analogous to that found in Compound 209, step 1 and Pd(PPh₃)₄ as the catalyst. ¹H NMR (400 MHz, DMSO-d₆) δ 8.58 (d, J=0.8 Hz, 1H), 8.13 (dd, J=8.2, 1.4 Hz, 1H), 8.02 (dd, J=8.4, 1.1 Hz, 1H), 7.87 (ddd, J=8.5, 6.9, 1.5 Hz, 1H), 7.71 (ddd, J=8.2, 6.9, 1.2 Hz, 1H), 7.52-7.46 (m, 2H), 7.46-7.37 (m, 2H), 7.41-7.32 (m, 1H), 7.25 (d, J=2.3 Hz, 1H), 7.06 (d, J=2.2 Hz, 1H), 5.26 (s, 2H), 2.51 (s, 3H). ESI-MS m/z calc. 360.10, found 361.2 (M+1)⁺.

### Step 2: 2-[2-[(3S,4S)-3-methoxy-4-methyl-pyrrolidin-1-yl]-3-quinolyl]-6-methyl-1H-pyridin-4-one, Compound 253

A mixture of 3-(4-benzyloxy-6-methyl-2-pyridyl)-2-chloro-quinoline (15 mg, 0.042 mmol), cesium carbonate (30 mg, 0.092 mmol), (3S,4S)-3-methoxy-4-methyl-pyrrolidine hydrochloride (10 mg, 0.066 mmol) and XPhos Pd G3 (4 mg, 0.005 mmol) in toluene (400 μL) was bubbled with nitrogen for 5 min. The mixture was sealed and stirred at 90° C. for 4 h. Filtration and purification by reverse phase HPLC (C18, 1-99% acetonitrile/5 mM HCl) provided the benzyl protected intermediate 3-(4-benzyloxy-6-methyl-2-pyridyl)-2-[(3S,4S)-3-methoxy-4-methyl-pyrrolidin-1-yl]quinoline (15.7 mg, 86%). ESI-MS m/z calc. 439.23, found 440.3 (M+1)⁺. The intermediate was dissolved in ethyl acetate (2 mL)/ethanol (0.2 mL)/TEA (5 L) and stirred with 10% Pd/C (10 mg, 0.009 mmol) under hydrogen atmosphere for 30 min. Filtration and purification by reverse phase HPLC (C18, 1-99% acetonitrile/5 mM HCl) provided 2-[2-[(3S,4S)-3-methoxy-4-methyl-pyrrolidin-1-yl]-3-quinolyl]-6-methyl-1H-pyridin-4-one (253, 9.5 mg, 64%). ¹H NMR (400 MHz, DMSO-d₆) δ 8.52 (s, 1H), 8.06 (s, 1H), 7.91 (d, J=7.9 Hz, 1H), 7.81 (t, J=7.8 Hz, 1H), 7.46 (t, J=7.5 Hz, 1H), 7.34 (s, 1H), 7.24 (d, J=2.3 Hz, 1H), 3.84-3.23 (m, 4H, obscured by water peak), 3.41 (s, 1H), 3.23 (s, 3H), 2.65 (s, 3H), 2.37 (s, 1H), 1.00 (d, J=6.7 Hz, 3H). ESI-MS m/z calc. 349.18, found 350.3 (M+1)⁺.

### Compound 254

### 2-[4-(3,4-difluoro-2-methyl-phenoxy)-6-(trifluoromethyl)-3-pyridyl]-6-methyl-1H-pyridin-4-one

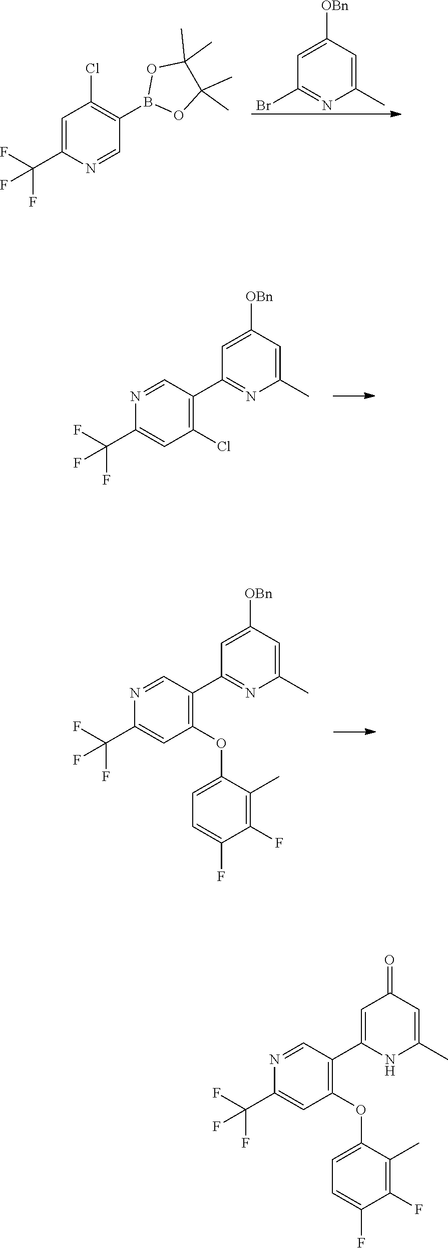

### Step 1: 4-benzyloxy-2-[4-chloro-6-(trifluoromethyl)-3-pyridyl]-6-methyl-pyridine

4-Benzyloxy-2-[4-chloro-6-(trifluoromethyl)-3-pyridyl]-6-methyl-pyridine was prepared from 4-chloro-5-(4,4,5,5-tetramethyl-1,3,2-dioxaborolan-2-yl)-2-(trifluoromethyl)pyridine and 4-benzyloxy-2-bromo-6-methyl-pyridine using a cross-coupling procedure analogous to that found in Compound 209, step 1 using Pd(PPh₃)₄ as the catalyst and DME as solvent. ESI-MS m/z calc. 378.08, found 379.305 (M+1)⁺.

### Step 2: 2-[4-(3,4-difluoro-2-methyl-phenoxy)-6-(trifluoromethyl)-3-pyridyl]-6-methyl-1H-pyridin-4-one, Coumpound 254

A mixture of 4-benzyloxy-2-[4-chloro-6-(trifluoromethyl)-3-pyridyl]-6-methyl-pyridine (19 mg, 0.043 mmol), 3,4-difluoro-2-methyl-phenol (15 mg, 0.10 mmol), 2-(2-methylpropanoyl) cyclohexanone (3.6 mg, 0.021 mmol), potassium carbonate (21 mg, 0.15 mmol) and copper (I) iodide (3.3 mg, 0.017 mmol) in DMSO (80 μL) was degassed with nitrogen and heated to 100° C. for 1 hour. The mixture was cooled to room temperature, diluted with ethyl acetate (30 mL) and washed with water (30 mL) and brine (30 mL). The organic phase was dried over sodium sulfate, filtered and concentrated. Purification by silica gel chromatography (0-100% ethyl acetate/hexanes) provided 4-benzyloxy-2-[4-(3,4-difluoro-2-methyl-phenoxy)-6-(trifluoromethyl)-3-pyridyl]-6-methyl-pyridine (14.5 mg, 70%). ESI-MS m/z calc. 486.14, found 487.2 (M+1)⁺. The benzyl-protected intermediate was dissolved in ethanol and stirred with 10% Pd/C under hydrogen atmosphere for 30 min. Filtration and HPLC purification (C18, 1-100% acetonitrile/water over 20 minutes) provided 2-[4-(3,4-difluoro-2-methyl-phenoxy)-6-(trifluoromethyl)-3-pyridyl]-6-methyl-1H-pyridin-4-one (254, 4.3 mg, 25%) as a white solid. ESI-MS m/z calc. 396.09, found 397.4 (M+1)⁺. ¹H NMR (500 MHz, DMSO-d₆) δ 8.99 (s, 1H), 7.49-7.42 (m, 1H), 7.41 (s, 1H), 7.23 (dd, J=9.5, 3.9 Hz, 1H), 7.19 (s, 1H), 7.09 (s, 1H), 2.62 (s, 3H), 2.11 (s, 3H).

### Compound 255 (trans isomer) and Compound 256 (cis isomer)

### 2-methyl-6-[5-(trifluoromethyl)-2-[4-(trifluoromethyl)cyclohexen-1-yl]-3-pyridyl]-1H-pyridin-4-one (trans) and 2-methyl-6-[5-(trifluoromethyl)-2-[4-(trifluoromethyl)cyclohexen-1-yl]-3-pyridyl]-1H-pyridin-4-one (cis)

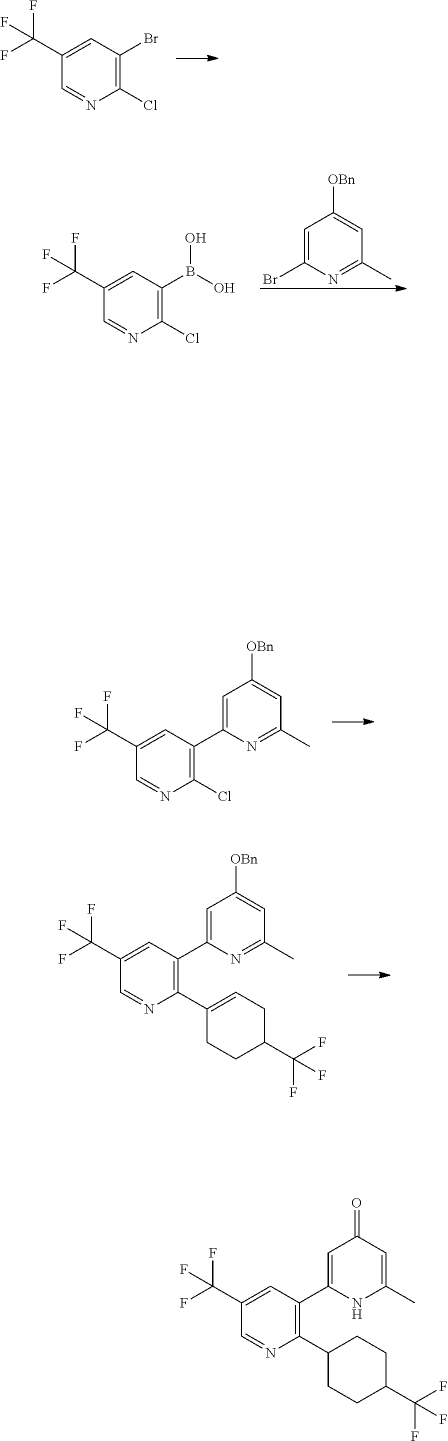 [SMILES: FC(F)(F)c1cnc(Cl)c(Br)c1 ; OB(O)c1cc(C(F)(F)F)cnc1Cl ; Cc1cc(OCc2ccccc2)cc(Br)n1 ; Cc1cc(OCc2ccccc2)cc(-c2cc(C(F)(F)F)cnc2Cl)n1 ; Cc1cc(OCc2ccccc2)cc(-c2cc(C(F)(F)F)cnc2C2=CCC(C(F)(F)F)CC2)n1 ; Cc1cc(=O)cc(-c2cc(C(F)(F)F)cnc2C2CCC(C(F)(F)F)CC2)[nH]1]

### Step 1: [2-chloro-5-(trifluoromethyl)-3-pyridyl]boronic acid

A solution of n-BuLi (3.4 mL of 2.5 M in hexanes, 8.5 mmol) diluted in diethyl ether (12 mL) was added dropwise to a solution of 3-bromo-2-chloro-5-(trifluoromethyl)pyridine (2.02 g, 7.7 mmol) in diethyl ether (20 mL) at −78° C. The mixture was stirred for 1 hour, then a solution of triisopropyl borate (2.4 mL, 10.5 mmol) in diethyl ether (10 mL) was slowly added at −78° C. over 10 min. The reaction mixture was removed from the dry ice bath and allowed to come to room temperature and stirred for 16 h. The mixture was cooled to 0° C., diluted with saturated ammonium chloride and the layers separated. The organic layer was extracted with aqueous 1 M NaOH (75 mL). The aqueous layer was washed with diethyl ether, and then acidified with aqueous HCl (115 mL) and extracted with diethyl ether (3×). The combined organic extracts were dried over sodium sulfate, filtered and concentrated to provide [2-chloro-5-(trifluoromethyl)-3-pyridyl]boronic acid (1.45 g, 83%). ESI-MS m/z calc. 225.00, found 225.9 (M+1)⁺.

Step 2: 3-(4-benzyloxy-6-methyl-2-pyridyl)-2-chloro-5-(trifluoromethyl)pyridine 3-(4-Benzyloxy-6-methyl-2-pyridyl)-2-chloro-5-(trifluoromethyl)pyridine was prepared from 4-benzyloxy-2-bromo-6-methyl-pyridine and [2-chloro-5-(trifluoromethyl)-3-pyridyl]boronic acid using a cross-coupling procedure analogous to that found in Compound 209, step 1 and Pd(PPh₃)₄ as the catalyst. ESI-MS m/z calc. 378.08, found 379.1 (M+1)⁺. ¹H NMR (500 MHz, DMSO-d₆) δ 8.92 (s, 1H), 8.37 (s, 1H), 7.52-7.46 (m, 2H), 7.46-7.40 (m, 2H), 7.39-7.32 (m, 1H), 7.29 (s, 1H), 7.07 (s, 1H), 5.26 (s, 2H), 2.50 (s, 3H).

### Step 3: 2-methyl-6-[5-(trifluoromethyl)-2-[4-(trifluoromethyl)cyclohexen-1-yl]-3-pyridyl]-1H-pyridin-4-one

3-(4-Benzyloxy-6-methyl-2-pyridyl)-5-(trifluoromethyl)-2-[4-(trifluoromethyl)cyclohexen-1-yl]pyridine was prepared from 3-(4-benzyloxy-6-methyl-2-pyridyl)-2-chloro-5-(trifluoromethyl)pyridine and 4,4,5,5-tetramethyl-2-[4-(trifluoromethyl)cyclohexen-1-yl]-1,3,2-dioxaborolane using a cross-coupling procedure analogous to that found in Compound 209, step 1. ESI-MS m/z calc. 492.16, found 493.3 (M+1)⁺. The benzyl-protected intermediate was dissolved in methanol and stirred vigorously with 10% Pd/C under hydrogen atmosphere for 16 h. The mixture was filtered, concentrated and purified by HPLC (10-70% acetonitrile/5 mM HCl) to provide the cis and trans isomers of 2-methyl-6-[5-(trifluoromethyl)-2-[4-(trifluoromethyl)cyclohexen-1-yl]-3-pyridyl]-1H-pyridin-4-one.

Compound 255, Peak 1 (minor): 2-methyl-6-[5-(trifluoromethyl)-2-[4-(trifluoromethyl)cyclohexen-1-yl]-3-pyridyl]-1H-pyridin-4-one (trans isomer) (10 mg, 28%). ESI-MS m/z calc. 404.13, found 405.3 (M+1)⁺; Retention time: 1.77 min. ¹H NMR (400 MHz, DMSO-d₆) δ 9.14 (d, J=2.0 Hz, 1H), 8.35 (s, 1H), 7.17 (s, 1H), 7.13 (s, 1H), 2.78 (t, J=11.6 Hz, 1H), 2.59 (s, 3H), 2.42-2.25 (m, 1H), 1.96-1.84 (m, 4H), 1.83-1.70 (m, 2H), 1.35-1.21 (m, 3H).

Compound 256, Peak 2 (major): 2-methyl-6-[5-(trifluoromethyl)-2-[4-(trifluoromethyl)cyclohexen-1-yl]-3-pyridyl]-1H-pyridin-4-one (cis isomer) (18 mg, 50%). ESI-MS m/z calc. 404.13, found 405.3 (M+1)⁺; Retention time: 1.83 min. ¹H NMR (400 MHz, DMSO-d₆) δ 14.20 (br s, 1H), 9.17 (d, J=2.3 Hz, 1H), 8.35 (d, J=2.3 Hz, 1H), 7.23 (s, 1H), 7.17 (s, 1H), 2.99 (d, J=9.4 Hz, 1H), 2.60 (s, 3H), 2.48-2.37 (m, 1H), 2.01-1.80 (m, 4H), 1.74-1.51 (m, 4H).

Retention times were determined by reversed phase UPLC using Acquity UPLC BEH C18 column (50×2.1 mm, 1.7 m) and a dual gradient run from 1-99% mobile phase B over 4.5 min. Mobile phase A=water (0.05% TFA). Mobile phase B=acetonitrile (0.035% TFA). Flow rate=1.2 mL/min, column temperature=60° C.

### Compound 257

### 2-methyl-6-[6-(trifluoromethyl)-2-[4-(trifluoromethyl)cyclohexyl]-3-pyridyl]-1H-pyridin-4-one

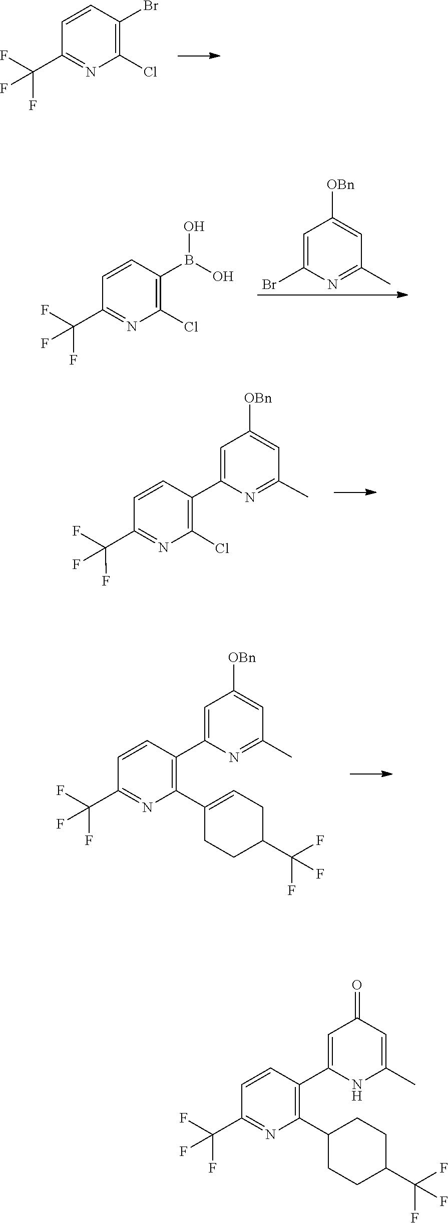 [SMILES: FC(F)(F)c1ccc(Br)c(Cl)n1 ; OB(O)c1ccc(C(F)(F)F)nc1Cl ; Cc1cc(OCc2ccccc2)cc(Br)n1 ; Cc1cc(OCc2ccccc2)cc(-c2ccc(C(F)(F)F)nc2Cl)n1 ; Cc1cc(OCc2ccccc2)cc(-c2ccc(C(F)(F)F)nc2C2=CCC(C(F)(F)F)CC2)n1 ; Cc1cc(=O)cc(-c2ccc(C(F)(F)F)nc2C2CCC(C(F)(F)F)CC2)[nH]1]

### Step 1: [2-chloro-6-(trifluoromethyl)-3-pyridyl]boronic acid

[2-Chloro-6-(trifluoromethyl)-3-pyridyl]boronic acid was prepared from 3-bromo-2-chloro-6-(trifluoromethyl)pyridine using a procedure analogous to that found in Compound 255, step 1. ESI-MS m/z calc. 225.00, found 226.0 (M+1)⁺.

### Step 2: 3-(4-benzyloxy-6-methyl-2-pyridyl)-2-chloro-6-(trifluoromethyl)pyridine

3-(4-Benzyloxy-6-methyl-2-pyridyl)-2-chloro-6-(trifluoromethyl)pyridine was prepared from 4-benzyloxy-2-bromo-6-methyl-pyridine and [2-chloro-6-(trifluoromethyl)-3-pyridyl]boronic acid using a cross-coupling procedure analogous to that found in Compound 209, step 1. ¹H NMR (400 MHz, DMSO-d₆) δ 8.28 (dd, J=7.8, 0.8 Hz, 1H), 8.06 (d, J=7.8 Hz, 1H), 7.51-7.44 (m, 2H), 7.46-7.38 (m, 2H), 7.42-7.32 (m, 1H), 7.27 (d, J=2.3 Hz, 1H), 7.08 (d, J=2.2 Hz, 1H), 5.25 (s, 2H), 2.51 (d, J=1.9 Hz, 3H). ESI-MS m/z calc. 378.08, found 379.2 (M+1)⁺.

Step 3: 2-methyl-6-[6-(trifluoromethyl)-2-[4-(trifluoromethyl)cyclohexyl]-3-pyridyl]-1H-pyridin-4-one, Compound 257.

3-(4-Benzyloxy-6-methyl-2-pyridyl)-6-(trifluoromethyl)-2-[4-(trifluoromethyl)cyclohexen-1-yl]pyridine was prepared from 3-(4-benzyloxy-6-methyl-2-pyridyl)-2-chloro-6-(trifluoromethyl)pyridine and 4,4,5,5-tetramethyl-2-[4-(trifluoromethyl)cyclohexen-1-yl]-1,3,2-dioxaborolane using a cross-coupling procedure analogous to that found in Compound 209, step 1. ESI-MS m/z calc. 492.16, found 493.4 (M+1)⁺.

The benzyl protected intermediate was dissolved in methanol and stirred with 10% Pd/C under hydrogen atmosphere for 1 h. Filtration and HPLC purification (C18, 1-99% acetonitrile/5 mM HCl) provided 2-methyl-6-[6-(trifluoromethyl)-2-[4-(trifluoromethyl)cyclohexyl]-3-pyridyl]-1H-pyridin-4-one (257, 8.7 mg, 30%). ¹H NMR (500 MHz, DMSO-d₆) δ 8.17 (t, J=8.0 Hz, 1H), 7.97 (dd, J=8.0, 5.0 Hz, 1H), 7.35-7.06 (m, 2H), 3.09 (p, J=5.0 Hz, 1H), 2.62 (d, J=3.2 Hz, 3H), 2.38 (d, J=10.8 Hz, 1H), 2.06-1.80 (m, 4H), 1.74-1.59 (m, 3H), 1.41-1.12 (m, 1H). ESI-MS m/z calc. 404.13, found 405.3 (M+1)⁺.

### Example 6

### Compound 258

### ethyl 2-[2-(3,4-difluoro-2-methyl-phenoxy)-3-quinolyl]-5-fluoro-6-methyl-4-oxo-1H-pyridine-3-carboxylate

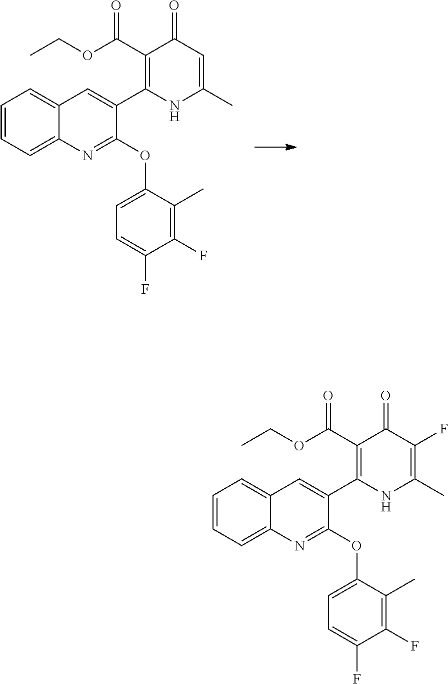 [SMILES: CCOC(=O)c1c(-c2cc3ccccc3nc2Oc2ccc(F)c(F)c2C)[nH]c(C)cc1=O ; CCOC(=O)c1c(-c2cc3ccccc3nc2Oc2ccc(F)c(F)c2C)[nH]c(C)c(F)c1=O]

A vial was charged with ethyl 2-[2-(3,4-difluoro-2-methyl-phenoxy)-3-quinolyl]-6-methyl-4-oxo-1H-pyridine-3-carboxylate (Compound 121, 38.6 mg, 0.0778 mmol), Selectfluor (84 mg, 0.24 mmol), and acetonitrile (250 L) under nitrogen atmosphere. In a separate 10 mL vial, [Pd(acetonitrile)4](BF₄)₂ (18.8 mg, 0.0423 mmol), 2,6-bis(2-pyridyl)pyridine (10 mg, 0.043 mmol), 1,10-phenanthroline (8.8 mg, 0.049 nmol) and acetonitrile (500 L) were added. The catalyst-containing solution was split in half and the first half was added to the mixture of ethyl 2-[2-(3,4-difluoro-2-methyl-phenoxy)-3-quinolyl]-6-methyl-4-oxo-1H-pyridine-3-carboxylate and Selectfluor. The resulting mixture was stirred at 25° C. for 2 h and 50° C. for 78 h. The mixture was allowed to cool to room temperature and diluted with ethyl acetate. The organic solution was washed with saturated aqueous ammonium chloride and brine. The organic layer was dried over magnesium sulfate, filtered, and concentrated under reduced pressure. Purification by reverse phase HPLC (C18, 10-99% acetonitrile/5 mM HCl over 20 min), followed by SFC purification (ChiralPak AS column, 55° C., isocratic methanol+20 mM NH₃, 1.8 mL/min) provided ethyl 2-[2-(3,4-difluoro-2-methyl-phenoxy)-3-quinolyl]-5-fluoro-6-methyl-4-oxo-1H-pyridine-3-carboxylate (258, 1 mg, 3%). ¹H NMR (400 MHz, CD₃OD) δ 8.13 (s, 1H), 7.86 (d, J=8.0 Hz, 1H), 7.69-7.57 (m, 2H), 7.45 (t, J=7.3 Hz, 1H), 7.16-7.05 (m, 1H), 7.05-6.95 (m, 1H), 4.14-3.84 (m, 2H), 2.37 (s, 3H), 2.07 (s, 3H), 0.81-0.67 (m, 3H). ¹⁹F NMR (376 MHz, CD₃OD) δ −142.17 (d, J=20.6 Hz), −145.19 (d, J=20.5 Hz), −154.23 (br s). ESI-MS m/z calc. 468.13, found 469.628 (M+1)⁺.

### Compound 259

### ethyl 5-chloro-2-[2-(4,4-difluoroazepan-1-yl)-5-methyl-6-(trifluoromethyl)-3-pyridyl]-6-methyl-4-oxo-1H-pyridine-3-carboxylate

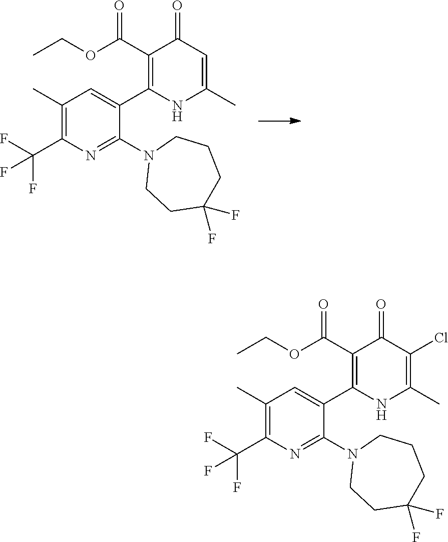 [SMILES: CCOC(=O)c1c(-c2cc(C)c(C(F)(F)F)nc2N2CCCC(F)(F)CC2)[nH]c(C)cc1=O ; CCOC(=O)c1c(-c2cc(C)c(C(F)(F)F)nc2N2CCCC(F)(F)CC2)[nH]c(C)c(Cl)c1=O]

Ethyl 2-[2-(4,4-difluoroazepan-1-yl)-5-methyl-6-(trifluoromethyl)-3-pyridyl]-6-methyl-4-oxo-1H-pyridine-3-carboxylate (Compound 118, 28.3 mg, 0.0573 mmol) was dissolved in DCM (1 mL) and methanol (100 μL) then 1-chloropyrrolidine-2,5-dione (46 mg, 0.35 mmol) was added and the reaction was stirred overnight. Additional 1-chloropyrrolidine-2,5-dione (83 mg, 0.6216 mmol) was added and the mixture stirred for 3 h. The mixture was filtered and purified by reverse phase HPLC (C18, 10-99% acetonitrile/5 mM HCl over 20 min) to provide ethyl 5-chloro-2-[2-(4,4-difluoroazepan-1-yl)-5-methyl-6-(trifluoromethyl)-3-pyridyl]-6-methyl-4-oxo-1H-pyridine-3-carboxylate (Hydrochloride salt) (259, 2.3 mg, 7%). ¹H NMR (400 MHz, CD₃OD) δ 7.56 (s, 1H), 4.09 (q, J=7.1 Hz, 2H), 3.60-3.53 (m, 2H), 3.36-3.29 (m, 2H) overlaps with CD₂HOD, 2.51 (s, 3H), 2.36 (q, J=2.2 Hz, 3H), 2.34-2.20 (m, 2H), 2.06-1.89 (m, 2H), 1.89-1.79 (m, 2H), 1.01 (t, J=7.1 Hz, 3H). ¹⁹F NMR (376 MHz, CD₃OD) δ −67.07, −92.19 (br s). ESI-MS m/z calc. 507.14, found 508.4 (M+1)⁺.

### Compound 260

### 3-bromo-2-[2-(3,4-difluoro-2-methyl-phenoxy)-3-quinolyl]-5,6-dimethyl-1H-pyridin-4-one

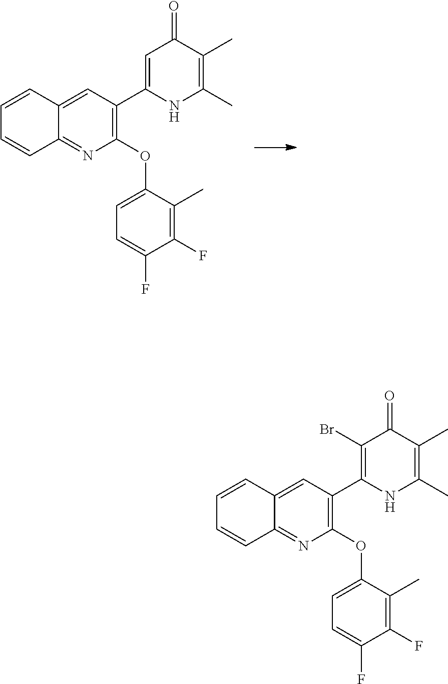 [SMILES: Cc1[nH]c(-c2cc3ccccc3nc2Oc2ccc(F)c(F)c2C)cc(=O)c1C ; Cc1[nH]c(-c2cc3ccccc3nc2Oc2ccc(F)c(F)c2C)c(Br)c(=O)c1C]

A suspension of 6-[2-(3,4-difluoro-2-methyl-phenoxy)-3-quinolyl]-2,3-dimethyl-1H-pyridin-4-one (Compound 141, 63 mg, 0.16 mmol) in DCM (1 mL) and acetic acid (0.5 mL) was cooled to 0° C. and treated with NBS (29 mg, 0.16 mmol) in a single portion. The mixture was removed from the ice bath and allowed to come to room temperature over 1 h, then heated at 35° C. for 1 h. The resulting solution was concentrated and purified by reverse phase HPLC (C18, 10-99% acetonitrile/5 mM HCl) to provide 3-bromo-2-[2-(3,4-difluoro-2-methyl-phenoxy)-3-quinolyl]-5,6-dimethyl-1H-pyridin-4-one (260, 25 mg, 33%). ESI-MS m/z calc. 470.04, found 471.3 (M+1)⁺. ¹H NMR (400 MHz, CD₃OD) δ 8.44 (s, 1H), 7.97 (d, J=8.2 Hz, 1H), 7.78-7.68 (m, 2H), 7.55 (ddd, J=8.1, 5.8, 2.3 Hz, 1H), 7.15 (q, J=9.2 Hz, 1H), 7.00 (ddd, J=9.1, 4.3, 2.0 Hz, 1H), 2.43 (s, 3H), 2.16 (s, 3H), 2.10 (d, J=2.2 Hz, 3H).

The following compounds were prepared using analogous chlorination conditions as found in Compound 259 or bromination conditions as found in Compound 260.

| | | MW & | |
|---|---|---|---|
| Cmpd | | Found | |
| No. | Compound Name | [M + H]⁺ | NMR (shifts in ppm) |
| 261 | methyl 5-chloro-6-[6-(3,3- | 518.14; | ¹H NMR (400 MHz, DMSO-d₆) δ 8.42 |
| | difluorocyclobutyl)-4-[4- | 519.3 | (s, 1H), 7.32 (s, 1H), 3.71 (s, 3H), 3.67- |
| | (trifluoromethyl)cyclohexyl]-3- | | 3.48 (m, 1H), 3.09-2.63 (m, 4H), |
| | pyridyl]-2-methyl-4-oxo-1H- | | 2.41-2.37 (m, 2H), 2.16 (s, 3H), 1.95- |
| | pyridine-3-carboxylate | | 1.72 (m, 2H), 1.73-1.37 (m, 6H). NH |
| | | | not observed. |
| 262 | ethyl 5-chloro-6-[6-(3,3- | 532.16; | ¹H NMR (400 MHz, DMSO-d₆) δ |
| | difluorocyclobutyl)-4-[4- | 533.3 | 12.20 (s, 1H), 8.53 (s, 1H), 7.42 (s, |
| | (trifluoromethyl)cyclohexyl]-3- | | 1H), 4.31-4.23 (m, 2H), 3.69-3.64 |
| | pyridyl]-2-methyl-4-oxo-1H- | | (m, 2H), 3.05-2.89 (m, 3H), 2.88- |
| | pyridine-3-carboxylate | | 2.72 (m, 1H), 2.47-2.39 (m, 1H), 2.25 |
| | | | (s, 3H), 1.99-1.87 (m, 2H), 1.72-1.56 |
| | | | (m, 6H), 1.28 (t, J = 7.1 Hz, 3H). |
| 263 | ethyl 5-bromo-2-[2-(3,4- | 528.05; | ¹H NMR (400 MHz, CD₃OD) δ 8.42 (s, |
| | difluoro-2-methyl-phenoxy)-3- | 529.1 | 1H), 7.94 (d, J = 8.2 Hz, 1H), 7.77- |
| | quinolyl]-6-methyl-4-oxo-1H- | | 7.66 (m, 2H), 7.58-7.49 (m, 1H), 7.17 |
| | pyridine-3-carboxylate | | (q, J = 9.3 Hz, 1H), 7.08-6.99 (m, 1H), |
| | | | 4.02 (q, J = 7.1 Hz, 2H), 2.60 (s, 3H), |
| | | | 2.09 (d, J = 2.2 Hz, 3H), 0.81 (t, J = 7.1 |
| | | | Hz, 3H). |
| 264 | 5-bromo-2-[2-(3,4-difluoro-2- | 486.04; | ¹H NMR (400 MHz, DMSO-d₆) δ |
| | methyl-phenoxy)-3-quinolyl]-3- | 487.11 | 12.16 (s, 1H), 8.61 (s, 1H), 8.05 (d, J = |
| | methoxy-6-methyl-1H-pyridin- | | 7.9 Hz, 1H), 7.74 (t, J = 7.7 Hz, 1H), |
| | 4-one | | 7.67 (d, J = 8.2 Hz, 1H), 7.57 (t, J = 7.5 |
| | | | Hz, 1H), 7.37 (q, J = 9.4 Hz, 1H), 7.09 |
| | | | (s, 1H), 3.71 (s, 3H), 2.47 (s, 3H), 2.04 |
| | | | (d, J = 2.0 Hz, 3H). |
| 265 | methyl 5-bromo-6-[2-(3,4- | 514.03; | ¹H NMR (400 MHz, CD₃OD) δ 8.48 (s, |
| | difluoro-2-methyl-phenoxy)-3- | 515.11 | 1H), 7.98 (d, J = 8.1 Hz, 1H), 7.73 (dd, |
| | quinolyl]-2-methyl-4-oxo-1H- | | J = 6.9, 1.7 Hz, 2H), 7.55 (ddd, J = 8.2, |
| | pyridine-3-carboxylate | | 6.1, 2.1 Hz, 1H), 7.16 (q, J = 9.3 Hz, |
| | | | 1H), 7.06-6.95 (m, 1H), 3.91 (s, 3H), |
| | | | 2.42 (s, 3H), 2.11 (d, J = 2.2 Hz, 3H). |
| 266 | 5-chloro-2-[2-(3,4-difluoro-2- | 470.12; | ¹H NMR (400 MHz, CD₃OD) δ 8.45 (s, |
| | methyl-phenoxy)-3-quinolyl]-3- | 471.2 | 1H), 7.94 (d, J = 8.1 Hz, 1H), 7.79- |
| | (ethoxymethyl)-6-methyl-1H- | | 7.67 (m, 2H), 7.61-7.47 (m, 1H), 7.25- |
| | pyridin-4-one | | 7.08 (m, 1H), 7.08-6.97 (m, 1H), |
| | | | 4.37 (s, 2H), 3.35-3.33 (m, 2H), 2.52 |
| | | | (s, 3H), 2.08 (d, J = 2.2 Hz, 3H), 0.94 |
| | | | (t, J = 7.0 Hz, 3H). |
| 267 | ethyl 5-chloro-2-[2-(4,4- | 475.15; | ¹H NMR (400 MHz, CD₃OD) δ 8.49 (s, |
| | difluoroazepan-1-yl)-3- | 476.4 | 1H), 8.06 (d, J = 8.6 Hz, 1H), 7.98- |
| | quinolyl]-6-methyl-4-oxo-1H- | | 7.87 (m, 2H), 7.61 (app t, J = 7.5 Hz, |
| | pyridine-3-carboxylate | | 1H), 4.14 (q, J = 7.1 Hz, 2H), 3.91- |
| | | | 3.80 (m, 2H), 3.73 (br s, 2H), 2.59 (s, |
| | | | 3H), 2.43-2.29 (m, 2H), 2.23-2.08 |
| | | | (m, 2H), 2.02-1.92 (m, 2H), 1.00 (t, J = |
| | | | 7.1 Hz, 3H). |
| 268 | 5-chloro-2-[2-(4,4- | 433.14; | ¹H NMR (400 MHz, CD₃OD) δ 8.82 (s, |
| | difluoroazepan-1-yl)-3- | 434.24 | 1H), 8.20 (d, J = 8.5 Hz, 1H), 8.07 (dd, |
| | quinolyl]-3-methoxy-6-methyl- | | J = 8.1, 1.3 Hz, 1H), 8.04-7.96 (m, |
| | 1H-pyridin-4-one | | 1H), 7.69 (t, J = 7.6 Hz, 1H), 3.86- |
| | | | 3.79 (m, 2H), 3.77 (s, 3H), 3.73-3.62 |
| | | | (m, 2H), 2.75 (s, 3H), 2.44-2.28 (m, |
| | | | 2H), 2.27-2.08 (m, 2H), 2.05-1.92 |
| | | | (m, 2H). |
| 269 | ethyl 5-bromo-6-[2-(3,4- | 528.05; | ¹H NMR (400 MHz, DMSO-d₆) δ |
| | difluoro-2-methyl-phenoxy)-3- | 529.1 | 12.44 (s, 1H), 8.66 (s, 1H), 8.10-8.02 |
| | quinolyl]-2-methyl-4-oxo-1H- | | (m, 1H), 7.81-7.73 (m, 1H), 7.69 (d, J = |
| | pyridine-3-carboxylate | | 8.3 Hz, 1H), 7.59 (t, J = 7.5 Hz, 1H), |
| | | | 7.38 (q, J = 9.4 Hz, 1H), 7.06 (dd, J = |
| | | | 9.2, 4.1 Hz, 1H), 4.27 (q, J = 7.1 Hz, |
| | | | 2H), 2.28 (s, 3H), 2.07 (d, J = 2.0 Hz, |
| | | | 3H), 1.28 (t, J = 7.1 Hz, 3H). |
| 270 | ethyl 5-chloro-2-[2-(3,4- | 484.88; | ¹H NMR (400 MHz, CD₃OD) δ 8.41 (s, |
| | difluoro-2-methyl-phenoxy)-3- | 485.69 | 1H), 7.94 (app d, J = 8.1 Hz, 1H), 7.77- |
| | quinolyl]-6-methyl-4-oxo-1H- | | 7.66 (m, 2H), 7.54 (ddd, J = 8.1, 6.2, |
| | pyridine-3-carboxylate | | 2.0 Hz, 1H), 7.17 (app q, J = 9.2 Hz, |
| | | | 1H), 7.03 (ddd, J = 9.1, 4.3, 2.1 Hz, |
| | | | 1H), 4.02 (q, J = 7.1 Hz, 2H), 2.56 (s, |
| | | | 3H), 2.09 (d, J = 2.2 Hz, 3H), 0.81 (t, J = |
| | | | 7.1 Hz, 3H). |
| 271 | 3,5-dichloro-2-[5-chloro-2-(3,4- | 512.97; | ¹H NMR (500 MHz, DMSO-d₆) δ |
| | difluoro-2-methoxy-phenoxy)- | 513.9 | 12.56 (s, 1H), 8.10 (s, 1H), 7.28 (s, |
| | 4-(trifluoromethyl)phenyl]-6- | | 1H), 7.22 (d, J = 9.3 Hz, 1H), 6.99 (s, |
| | methyl-1H-pyridin-4-one | | 1H), 3.79 (d, J = 1.2 Hz, 3H), 2.41 (s, |
| | | | 3H). |

### Compound 272

### ethyl 2-[2-(3,4-difluoro-2-methyl-phenoxy)-3-quinolyl]-5-(dimethylamino)-6-methyl-4-oxo-1H-pyridine-3-carboxylate

### Step 1: ethyl 4-benzyloxy-5-bromo-2-[2-(3,4-difluoro-2-methyl-phenoxy)-3-quinolyl]-6-methyl-pyridine-3-carboxylate

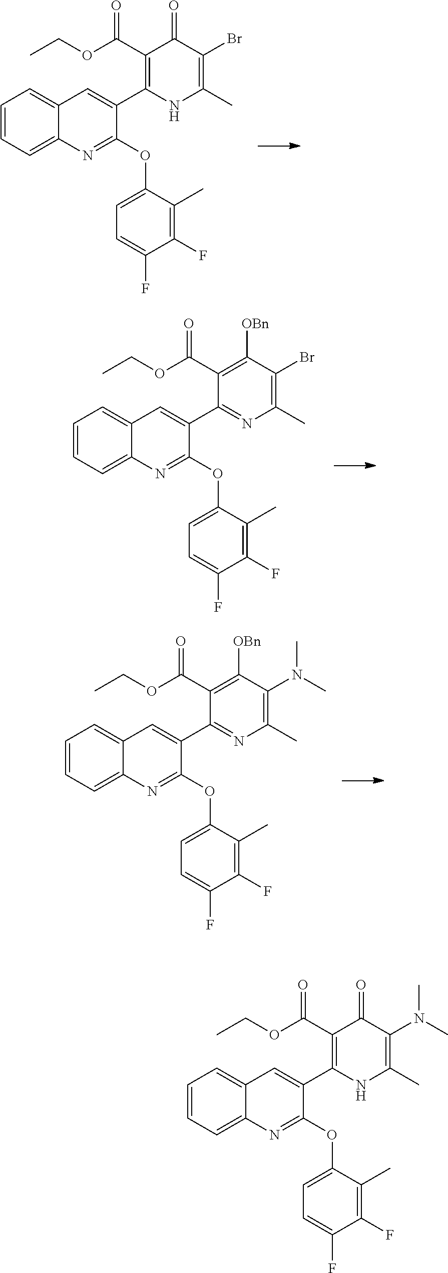

To a suspension of ethyl 5-bromo-2-[2-(3,4-difluoro-2-methyl-phenoxy)-3-quinolyl]-6-methyl-4-oxo-1H-pyridine-3-carboxylate (Compound 263, 83 mg, 0.16 mmol) and cesium carbonate (81 mg, 0.25 mmol) in acetonitrile (1.5 mL) was added benzylbromide (56 μL, 0.47 mmol). The reaction mixture was stirred at room temperature for 1 h, then diluted with additional acetonitrile (1.5 mL) and DMF (0.5 mL) and stirred for 2 h. The mixture was concentrated and partitioned between ethyl acetate and water. The aqueous layer was extracted with additional ethyl acetate (3×). The combine organic extracts was dried over sodium sulfate, filtered and concentrated. Purification by silica gel chromatography (0-30% ethyl acetate/hexanes) provided ethyl 4-benzyloxy-5-bromo-2-[2-(3,4-difluoro-2-methyl-phenoxy)-3-quinolyl]-6-methyl-pyridine-3-carboxylate (82.1 mg, 84%). ESI-MS m/z calc. 618.10, found 619.22 (M+1)^{m}.

### Step 2: ethyl 4-benzyloxy-2-[2-(3,4-difluoro-2-methyl-phenoxy)-3-quinolyl]-5-(dimethylamino)-6-methyl-pyridine-3-carboxylate

Ethyl 4-benzyloxy-5-bromo-2-[2-(3,4-difluoro-2-methyl-phenoxy)-3-quinolyl]-6-methyl-pyridine-3-carboxylate (82 mg, 0.13 mmol) and tBuXPhos Palladacycle Gen3 (25.7 mg, 0.0324 mmol) were added to an oven-dried 4 mL screw-top vial and the vessel was purged with nitrogen. THF (1 mL) and phosphazene base P₂-Et (Aldrich 79417, 111 μL, 0.334 mmol) were added and the resulting mixture was sparged for 2 min with nitrogen. Dimethylamine (332 μL of 2.0 M in THF, 0.66 mmol) was added under a nitrogen atmosphere. The mixture was sealed and stirred at 50° C. for 6 h. The mixture was diluted with ethyl acetate and washed with a saturated aqueous ammonium chloride. The aqueous layer was extracted with additional ethyl acetate, and the combined organic extracts washed with brine, dried over sodium sulfate, filtered and concentrated in vacuo. Purification by silica gel chromatography (0-100% ethyl acetate/hexanes) provided ethyl 4-benzyloxy-2-[2-(3,4-difluoro-2-methyl-phenoxy)-3-quinolyl]-5-(dimethylamino)-6-methyl-pyridine-3-carboxylate (7.7 mg, 10%). ESI-MS m % z calc. 583.23, found 584.4 (M+1)⁺.

### Step 3: ethyl 2-[2-(3,4-difluoro-2-methyl-phenoxy)-3-quinolyl]-5-(dimethylamino)-6-methyl-4-oxo-1H-pyridine-3-carboxylate, Compound 272

Ethyl 2-[2-(3,4-difluoro-2-methyl-phenoxy)-3-quinolyl]-5-(dimethylamino)-6-methyl-4-oxo-1H-pyridine-3-carboxylate was prepared from ethyl 4-benzyloxy-2-[2-(3,4-difluoro-2-methyl-phenoxy)-3-quinolyl]-5-(dimethylamino)-6-methyl-pyridine-3-carboxylate using a debenzylation procedure analogous to that found in Compound 209, step 3. ESI-MS m/z calc. 493.18, found 494.3 (M+1)⁺. ¹H NMR (400 MHz, DMSO-d₆) δ 8.48 (s, 1H), 8.02 (d, J=8.1 Hz, 1H), 7.78-7.68 (m, 1H), 7.68-7.59 (m, 1H), 7.59-7.49 (m, 1H), 7.44-7.30 (m, 1H), 7.14-7.02 (m, 1H), 3.97-3.79 (m, 2H), 3.17 (s, 3H), 2.80 (s, 6H), 2.39 (s, 3H), 0.71 (t, J=7.1 Hz, 3H).

### Compound 273

### 2-[4-tert-butyl-2-(4-fluoro-2-methoxy-phenoxy)-6-methyl-phenyl]-6-(hydroxymethyl)-1H-pyridin-4-one

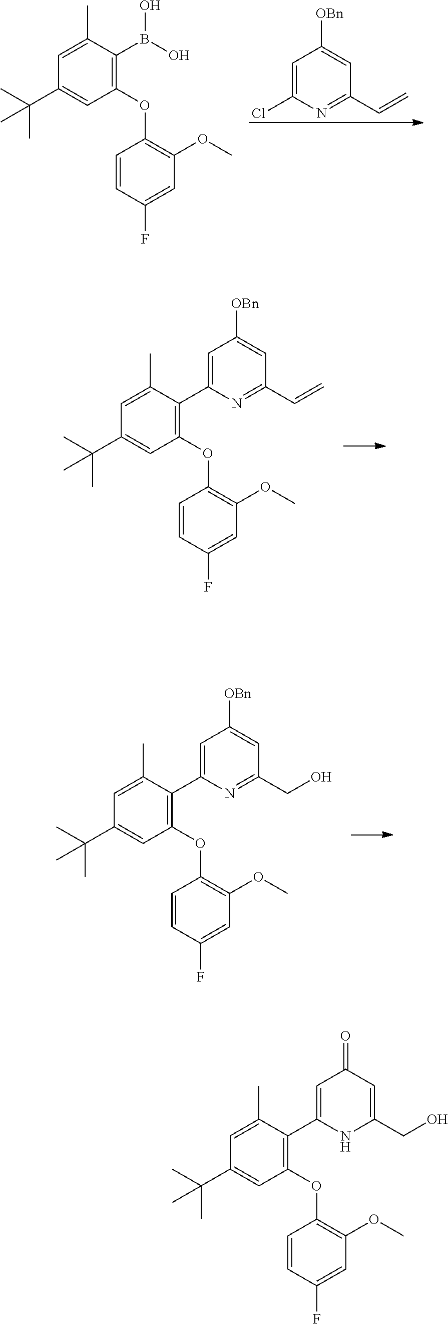

### Step 1: 4-benzyloxy-2-[4-tert-butyl-2-(4-fluoro-2-methoxy-phenoxy)-6-methyl-phenyl]-6-vinyl-pyridine

A solution of 4-benzyloxy-2-chloro-6-vinyl-pyridine (272 mg, 1.11 mmol) and [4-tert-butyl-2-(4-fluoro-2-methoxy-phenoxy)-6-methyl-phenyl]boronic acid (250 mg, 0.753 mmol) in 1,4-dioxane (8 mL) and aqueous potassium carbonate (2 mL of 2 M, 4 mmol) was bubbled with nitrogen for 5 min. Pd(PPh₃)₄ (87 mg, 0.075 mmol) was added and the mixture stirred at 90° C. for 2 h. The mixture was cooled, diluted with ethyl acetate and the layers separated. The organic layer was dried over sodium sulfate, filtered and concentrated. Purification by silica gel chromatography (40 g silica, dichloromethane) provided 4-benzyloxy-2-[4-tert-butyl-2-(4-fluoro-2-methoxy-phenoxy)-6-methyl-phenyl]-6-vinyl-pyridine (145 mg, 39%). ESI-MS m/z calc. 497.24, found 498.4 (M+1)⁺. ¹H NMR (400 MHz, DMSO-d₆) δ 7.46-7.42 (m, 2H), 7.41-7.31 (m, 3H), 7.08 (d, J=2.3 Hz, 1H), 7.04-7.01 (m, 2H), 7.00-6.98 (m, 1H), 6.95 (dd, J=8.8, 5.9 Hz, 1H), 6.79-6.69 (m, 2H), 6.50 (d, J=1.9 Hz, 1H), 6.19 (dd, J=17.4, 1.8 Hz, 1H), 5.42 (dd, J=10.7, 1.8 Hz, 1H), 5.18 (s, 2H), 3.70 (s, 3H), 2.10 (s, 3H), 1.19 (s, 9H).

### Step 2: 4-benzyloxy-6-[4-tert-butyl-2-(4-fluoro-2-methoxy-phenoxy)-6-methyl-phenyl]pyridine-2-carbaldehyde and [4-benzyloxy-6-[4-tert-butyl-2-(4-fluoro-2-methoxy-phenoxy)-6-methyl-phenyl]-2-pyridyl]methanol

To a solution of 4-benzyloxy-2-[4-tert-butyl-2-(4-fluoro-2-methoxy-phenoxy)-6-methyl-phenyl]-6-vinyl-pyridine (30 mg, 0.060 mmol) and 2,6-lutidine (15 μL, 0.13 mmol) in 1,4-dioxane (2 mL) was added OsO₄ (123 μL of 2.5% w/v in ^{t}BuOH, 0.0121 mmol) and the mixture was stirred for 5 min. A solution of NaIO₄ (52 mg, 0.24 mmol) in water (0.5 mL) was added and the mixture stirred for 20 min, then heated at 45° C. for 1 hour. The reaction was diluted with water and extracted with dichloromethane. The organic phase was dried over sodium sulfate, filtered and concentrated to provide 4-benzyloxy-6-[4-tert-butyl-2-(4-fluoro-2-methoxy-phenoxy)-6-methyl-phenyl]pyridine-2-carbaldehyde. The residue was dissolved in methanol (3 mL) and then treated with solid NaBH₄ (6.0 mg, 0.16 mmol). The reaction was stirred for 10 min, then partitioned between dichloromethane and water. The organic phase was separated, dried over sodium sulfate, filtered and concentrated to provide [4-benzyloxy-6-[4-tert-butyl-2-(4-fluoro-2-methoxy-phenoxy)-6-methyl-phenyl]-2-pyridyl]methanol (28 mg, 93%). ESI-MS m/z calc. 501.23, found 502.5 (M+1)⁺.

### Step 3: 2-[4-tert-butyl-2-(4-fluoro-2-methoxy-phenoxy)-6-methyl-phenyl]-6-(hydroxymethyl)-1H-pyridin-4-one, Compound 273

[4-Benzyloxy-6-[4-tert-butyl-2-(4-fluoro-2-methoxy-phenoxy)-6-methyl-phenyl]-2-pyridyl]methanol (28 mg, 0.056 mmol) was dissolved in methanol (3 mL) and stirred vigorously with 10% Pd/C (wet) (15 mg, 0.0071 mmol) under hydrogen atmosphere for 2 h. The mixture was filtered and concentrated in vacuo. Purification by HPLC (C18, 10-99% acetonitrile/5 mM HCl over 20 min) provided 2-[4-tert-butyl-2-(4-fluoro-2-methoxy-phenoxy)-6-methyl-phenyl]-6-(hydroxymethyl)-1H-pyridin-4-one (273, 12 mg, 52%). ESI-MS m/z calc. 411.19, found 412.3 (M+1)−. ¹H NMR (400 MHz, DMSO-d₆) δ 14.31 (s, 1H), 7.23 (d, J=2.4 Hz, 1H), 7.18-7.04 (m, 4H), 6.80 (td, J=8.5, 3.0 Hz, 1H), 6.54 (d, J=1.7 Hz, 1H), 6.03 (s, 1H), 4.67 (s, 2H), 3.72 (s, 3H), 2.17 (s, 3H), 1.19 (s, 9H).

### Compound 274

### 2-[4-tert-butyl-2-(4-fluoro-2-methoxy-phenoxy)-6-methyl-phenyl]-6-(1,2-dihydroxyethyl)-1H-pyridin-4-one

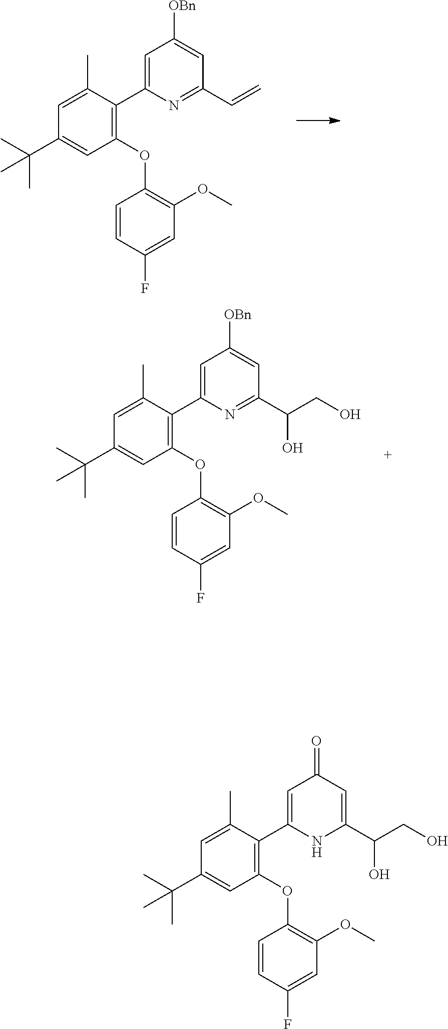

4-Benzyloxy-2-[4-tert-butyl-2-(4-fluoro-2-methoxy-phenoxy)-6-methyl-phenyl]-6-vinyl-pyridine (Compound 273, step 1, 37 mg, 0.074 mmol) was dissolved in acetone (1 mL) and water (0.2 mL) and treated with 4-methyl-4-oxido-morpholin-4-ium (24 μL of 4.8 M, 0.12 mmol) and OsO₄ (76 μL of 2.5% w/v in BuOH, 0.0075 mmol). The mixture was stirred at room temperature for 20 min, then partitioned between 10% aqueous sodium bisulfite and ethyl acetate. The organic layer was separated, dried over sodium sulfate, filtered and concentrated in vacuo to provide 1-[4-benzyloxy-6-[4-tert-butyl-2-(4-fluoro-2-methoxy-phenoxy)-6-methyl-phenyl]-2-pyridyl]ethane-1,2-diol. ESI-MS m/z calc. 531.24, found 532.4 (M+1)⁺. The residue was dissolved in methanol (2 mL) and stirred with 10% Pd/C (wet, 16 mg, 0.0075 mmol) under hydrogen atmosphere for 2 h. The mixture was filtered and purified by HPLC (C18, 10-99% acetonitrile/5 mM HCl over 20 min) to provide 2-[4-tert-butyl-2-(4-fluoro-2-methoxy-phenoxy)-6-methyl-phenyl]-6-(1,2-dihydroxyethyl)-1H-pyridin-4-one (274, 23 mg, 70%). ESI-MS m/z calc. 441.20, found 442.3 (M+1)⁺. ¹H NMR (400 MHz, DMSO-d₆) δ 14.29 (s, 1H), 7.25 (d, 2H), 7.17-7.12 (m, 2H), 7.09 (dd, J=10.7, 2.9 Hz, 1H), 6.80 (td, J=8.5, 2.9 Hz, 1H), 6.50 (d, J=1.7 Hz, 1H), 6.17 (s, 1H), 5.09 (s, 1H), 4.85-4.77 (m, 1H), 3.73 (s, 3H), 3.66 (dd, J=11.0, 5.4 Hz, 1H), 3.58 (dd, J=11.0, 5.6 Hz, 1H), 2.16 (s, 3H), 1.19 (s, 9H).

### Compound 275

### 2-[4-tert-butyl-2-(4-fluoro-2-methoxy-phenoxy)-6-methyl-phenyl]-6-(2-hydroxyethyl)-1H-pyridin-4-one

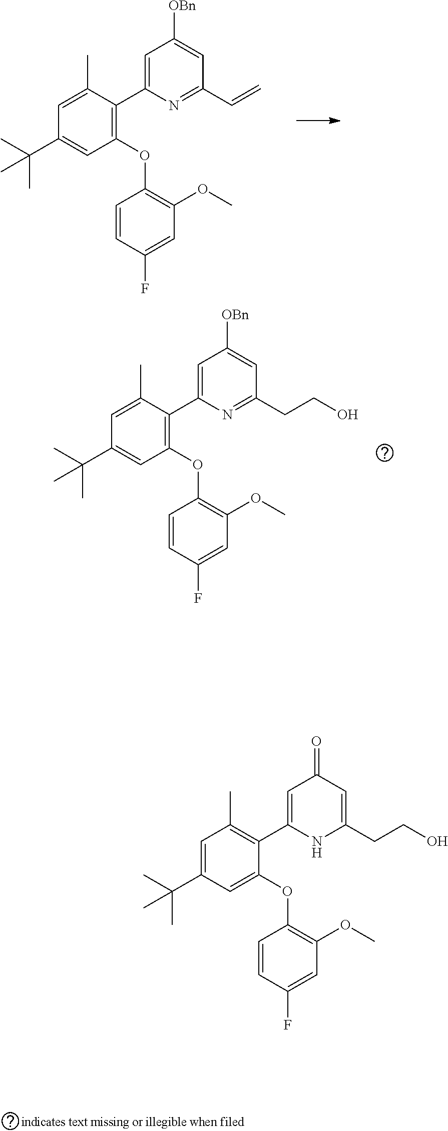

4-Benzyloxy-2-[4-tert-butyl-2-(4-fluoro-2-methoxy-phenoxy)-6-methyl-phenyl]-6-vinyl-pyridine (30 mg, 0.060 mmol) was dissolved in THF (200 L) and cooled to 0° C. under nitrogen. A solution of 9-BBN (480 μL of 0.5 M in THF, 0.24 mmol) was added dropwise to the stirring mixture. The mixture was stirred at room temperature for 2.5 h, then cooled to 0° C. and treated dropwise with aqueous NaOH (150 μL of 4 M, 0.60 mmol) followed by the dropwise addition of H₂O₂ (200 μL of 30% w/v, 1.8 mmol). The cooling bath was removed and the reaction stirred for 20 min. The mixture was partitioned between ethyl acetate and water. The organic layer was washed with water and brine, dried over sodium sulfate, filtered and concentrated in vacuo to provide 2-[4-benzyloxy-6-[4-tert-butyl-2-(4-fluoro-2-methoxy-phenoxy)-6-methyl-phenyl]-2-pyridyl]ethanol. ESI-MS m/z calc. 515.25, found 516.5 (M+1)⁺. The residue was dissolved in methanol (3 mL) and stirred vigorously with 10% Pd/C (wet) (13 mg, 0.0061 mmol) under hydrogen atmosphere for 10 min. The mixture was filtered and concentrated in vacuo. Purification by reverse phase HPLC (C18, 10-99% acetonitrile/5 mM HCl) provided 2-[4-tert-butyl-2-(4-fluoro-2-methoxy-phenoxy)-6-methyl-phenyl]-6-(2-hydroxyethyl)-1H-pyridin-4-one. ESI-MS m/z calc. 425.20, found 426.4 (M+1)⁺. ¹H NMR (400 MHz, DMSO-d₆) δ 14.34 (br s, 1H), 7.23 (d, J=2.4 Hz, 1H), 7.19 (d, J=2.4 Hz, 1H), 7.17 (dd, J=1.7, 0.8 Hz, 1H), 7.13 (dd, J=8.9, 5.9 Hz, 1H), 7.08 (dd, J=10.7, 2.9 Hz, 1H), 6.80 (td, J=8.5, 2.9 Hz, 1H), 6.53 (d, J=1.7 Hz, 1H), 3.77-3.72 (m, 5H), 3.01 (t, J=6.0 Hz, 2H), 2.18 (s, 3H), 1.19 (s, 9H).

### Compound 276

### 2-[4-tert-butyl-2-(4-fluoro-2-methoxy-phenoxy)-6-methyl-phenyl]-6-ethyl-1H-pyridin-4-one

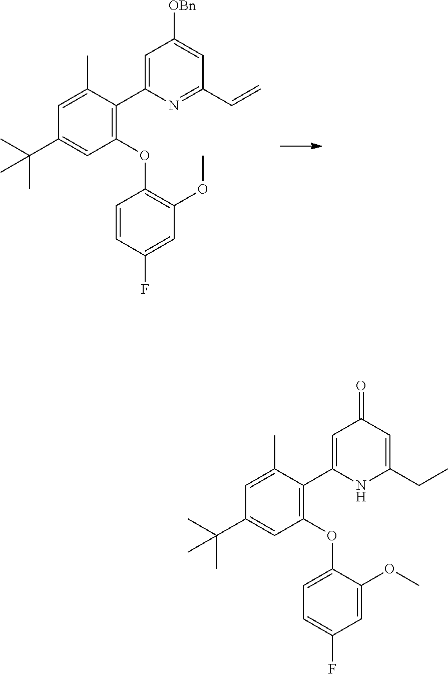 [SMILES: C=Cc1cc(OCc2ccccc2)cc(-c2c(C)cc(C(C)(C)C)cc2Oc2ccc(F)cc2OC)n1 ; CCc1cc(=O)cc(-c2c(C)cc(C(C)(C)C)cc2Oc2ccc(F)cc2OC)[nH]1]

2-[4-tert-Butyl-2-(4-fluoro-2-methoxy-phenoxy)-6-methyl-phenyl]-6-ethyl-1H-pyridin-4-one was prepared from 4-benzyloxy-2-[4-tert-butyl-2-(4-fluoro-2-methoxy-phenoxy)-6-methyl-phenyl]-6-vinyl-pyridine (Compound 273, step 1) using a debenzylation procedure analogous to that found in Compound 209, step 3. ¹H NMR (400 MHz, CD₃OD) δ 7.12 (s, 1H), 6.96 (dd, J=8.8, 5.6 Hz, 1H), 6.90 (dd, J=10.5, 2.9 Hz, 1H), 6.69 (dd, J=8.2, 2.8 Hz, 1H), 6.66-6.61 (m, 1H), 6.38-6.29 (m, 2H), 3.77 (s, 3H), 2.64 (q, J=7.6 Hz, 2H), 2.25 (s, 3H), 1.28 (t, J=7.6 Hz, 3H), 1.24 (s, 9H). ¹⁹F NMR (377 MHz, CD₃OD) δ 117.10-−117.28 (m, 1F). ESI-MS m/z calc. 409.21, found 410.2 (M+1)−.

### Compound 277 and Compound 278

### ethyl 3-[2-[2-(3,4-difluoro-2-methyl-phenoxy)-3-quinolyl]-6-methyl-4-oxo-1H-pyridin-3-yl]prop-2-enoate and ethyl 3-[2-[2-(3,4-difluoro-2-methyl-phenoxy)-3-quinolyl]-6-methyl-4-oxo-1H-pyridin-3-yl]propanoate

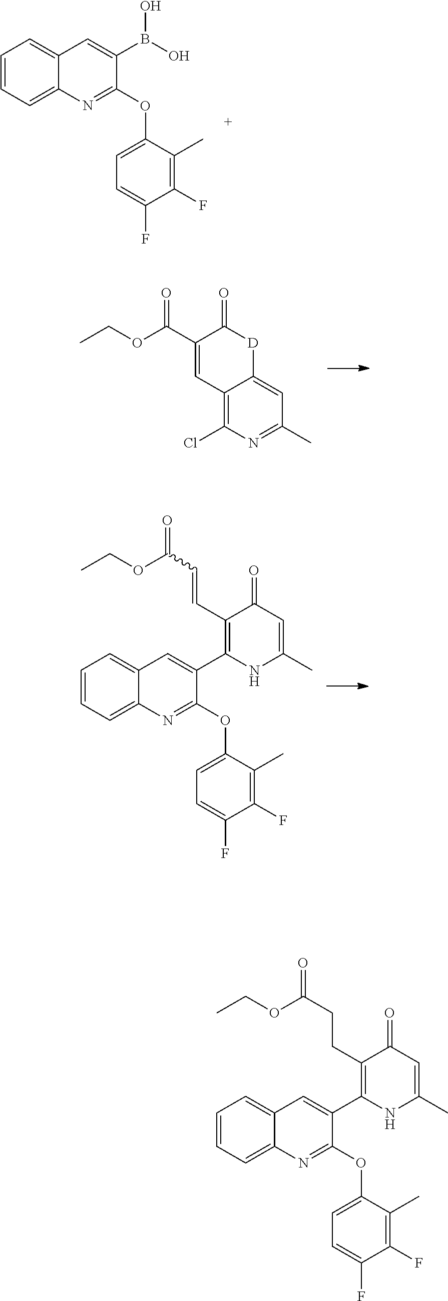

Step 1: A solution of ethyl 5-chloro-7-methyl-2-oxo-pyrano[3,2-c]pyridine-3-carboxylate (100 mg, 0.374 mmol), [2-(3,4-difluoro-2-methyl-phenoxy)-3-quinolyl]boronic acid (146 mg, 0.464 mmol), potassium carbonate (157 mg, 1.14 mmol), Pd(dppf)Cl₂ (33.7 mg, 0.0413 mmol) in DMSO (1.75 mL) and water (200 L) was sparged with nitrogen for 2 min and heated at 120° C. for 30 min under microwave irradiation. The mixture was partitioned between ethyl acetate and water and the layers separated. The aqueous layer was extracted with ethyl acetate, and the combined organic extracts washed with brine, dried over sodium sulfate, filtered and concentrated. Purification by silica gel chromatography (0-100% ethyl acetate/hexanes) provided ethyl 3-[2-[2-(3,4-difluoro-2-methyl-phenoxy)-3-quinolyl]-6-methyl-4-oxo-1H-pyridin-3-yl]prop-2-enoate (Compound 277, 13.8 mg, 7%). ESI-MS m/z calc. 476.16, found 477.3 (M+1)⁺. ¹H NMR (400 MHz, CD₃OD) δ 8.50 (s, 1H), 7.99 (d, J=8.0 Hz, 1H), 7.80-7.69 (m, 2H), 7.60-7.54 (m, 1H), 7.41 (d, J=15.6 Hz, 1H), 7.31 (d, J=15.6 Hz, 1H), 7.14 (q, J=9.4 Hz, 1H), 6.96-6.90 (m, 1H), 6.42 (s, 1H), 4.10 (q, J=7.1 Hz, 2H), 2.39 (s, 3H), 2.03 (d, J=2.2 Hz, 3H), 1.18 (t, J=7.1 Hz, 3H).

Step 2: Ethyl 3-[2-[2-(3,4-difluoro-2-methyl-phenoxy)-3-quinolyl]-6-methyl-4-oxo-1H-pyridin-3-yl]propanoate (Compound 278) was prepared from ethyl 3-[2-[2-(3,4-difluoro-2-methyl-phenoxy)-3-quinolyl]-6-methyl-4-oxo-1H-pyridin-3-yl]prop-2-enoate using a hydrogenation procedure analogous to that found in Compound 209, step 3. ¹H NMR (400 MHz, CD₃OD) δ 8.52 (s, 1H), 7.99 (d, J=8.0 Hz, 1H), 7.82-7.69 (m, 2H), 7.63-7.52 (m, 1H), 7.18 (q, J=9.3 Hz, 1H), 7.08-6.98 (m, 1H), 6.75 (s, 1H), 3.96 (q, J=7.1 Hz, 2H), 2.99-2.67 (m, 2H), 2.62-2.53 (m, 2H), 2.52 (s, 3H), 2.08 (d, J=2.2 Hz, 3H), 1.08 (t, J=7.1 Hz, 3H). ESI-MS m/z calc. 478.17, found 480.1 (M+1)⁺.

### Example 7

### Compound 279

### 6-[4-tert-butyl-2-(4-fluoro-2-methoxy-phenoxy)-6-methyl-phenyl]-2-methyl-4-oxo-1H-pyridine-3-carboxylic acid

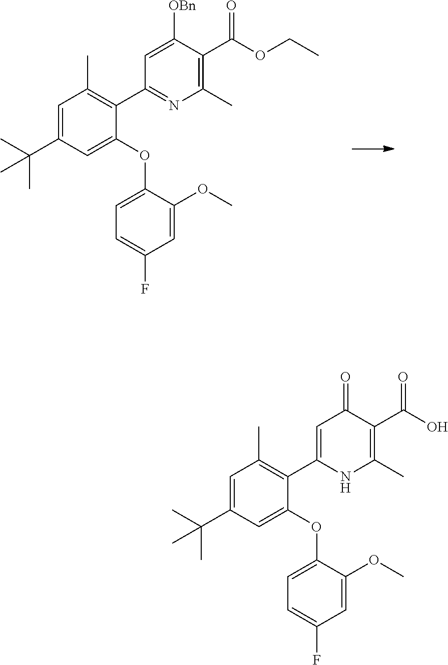

To a solution of ethyl 4-benzyloxy-6-[4-tert-butyl-2-(4-fluoro-2-methoxy-phenoxy)-6-methyl-phenyl]-2-methyl-pyridine-3-carboxylate (70 mg, 0.10 mmol) in methanol (0.6 mL), water (0.6 mL) and tetrahydrofuran (0.6 mL) was added lithium hydroxide (30 mg, 0.72 mmol). The mixture was stirred at room temperature for 5 h. Additional lithium hydroxide (28 mg, 0.67 mmol), methanol (0.6 mL), water (0.6 mL) and tetrahydrofuran (0.6 mL) were added and the mixture was stirred at 50° C. for 48 h. The mixture was diluted with ethyl acetate (75 mL), washed with 5% aqueous citric acid (10 mL) and brine (10 mL), dried over sodium sulfate, filtered and concentrated. Reverse phase purification (C18, 5-95% acetonitrile/water, each with 0.1% formic acid) provided 4-benzyloxy-6-[4-tert-butyl-2-(4-fluoro-2-methoxy-phenoxy)-6-methyl-phenyl]-2-methyl-pyridine-3-carboxylic acid (46 mg, 89%) as a white solid. ESI-MS m/z calc. 529.23, found 530.3 (M+1)⁺. ¹H NMR (400 MHz, DMSO-d₆) δ 13.37-12.45 (m, 1H), 7.41-7.25 (m, 5H), 7.13 (s, 1H), 7.04-6.98 (m, 2H), 6.94 (dd, J=8.7, 6.1 Hz, 1H), 6.74 (dt, J=8.5, 2.7 Hz, 1H), 6.49 (s, 1H), 5.18 (s, 2H), 3.71 (s, 3H), 2.40 (s, 3H), 2.07 (s, 3H), 1.18 (s, 9H). ¹⁹F NMR (377 MHz, DMSO-d₆) δ −115.82 (s, 1F).

6-[4-tert-Butyl-2-(4-fluoro-2-methoxy-phenoxy)-6-methyl-phenyl]-2-methyl-4-oxo-1H-pyridine-3-carboxylic acid was prepared from the benzyl-protected intermediate using a debenzylation procedure analogous to that found in Compound 209, step 3. ESI-MS m/z calc. 439.18, found 440.3 (M+1)⁺. ¹H NMR (400 MHz, DMSO-d₆) δ 12.80 (br s, 1H), 7.14 (s, 1H), 7.12-7.03 (m, 2H), 6.78 (td, J=8.4, 2.9 Hz, 1H), 6.67 (s, 1H), 6.53 (s, 1H), 3.72 (s, 3H), 2.76 (s, 3H), 2.20 (s, 3H), 1.19 (s, 9H). ¹⁹F NMR (377 MHz, DMSO-d₆) δ 114.62 (s, 1F).

The following compounds were synthesized from the corresponding ester using chemistry analogous to that found in Compound 279.

| | | MW & | |
|---|---|---|---|
| Cmpd | | Found | |
| No | Compound Name | [M + H]⁺ | NMR (shifts in ppm) |
| 280 | 2-[5-(3,4-difluoro-2- | 470.09, | ¹H NMR (500 MHz, DMSO-d₆) δ |
| | methoxy-phenoxy)-2- | 471.4 | 13.02 (s, 1H), 8.34 (s, 1H), 8.16 (s, |
| | (trifluoromethyl)-4- | | 1H), 7.22-7.19 (m, 1H), 6.96-6.93 |
| | pyridyl]-5,6-dimethyl-4- | | (m, 1H), 3.81 (s, 3H), 2.41 (s, 3H), |
| | oxo-1H-pyridine-3- | | 2.05 (s, 3H). |
| | carboxylic acid | | |
| 281 | 2-[6-[2-(3,4-difluoro-2- | 508.14, | ¹H NMR (400 MHz, CD₃OD) δ |
| | methyl-phenoxy)-3- | 509.4 | 8.49 (s, 1H), 7.98 (d, J = 8.1 Hz, |
| | quinolyl]-5- | | 1H), 7.81-7.69 (m, 2H), 7.57 (ddd, |
| | ethoxycarbonyl-2-methyl-4- | | J = 8.2, 6.4, 1.8 Hz, 1H), 7.18 (app |
| | oxo-1H-pyridin-3-yl]acetic | | q, J = 9.3 Hz, 1H), 6.99 (ddd, J = |
| | acid | | 9.1, 4.2, 2.0 Hz, 1H), 4.11 (q, J = 7.1 |
| | | | Hz, 2H), 3.84 (s, 2H), 2.62 (s, 3H), |
| | | | 2.07 (d, J = 2.2 Hz, 3H), 0.70 (t, J = |
| | | | 7.1 Hz, 3H) |
| 282 | 2-[6-[2-(4,4- | 499.19, | ¹H NMR (400 MHz, CD₃OD-d4) δ |
| | difluoroazepan-1-yl)-3- | 500.4 | 8.59 (s, 1H), 8.14 (d, J = 8.5 Hz, |
| | quinolyl]-5- | | 1H), 8.02-7.92 (m, 2H), 7.65 (t, J = |
| | ethoxycarbonyl-2-methyl-4- | | 7.6 Hz, 1H), 4.20 (q, J = 7.0 Hz, |
| | oxo-1H-pyridin-3-yl]acetic | | 2H), 3.88-3.76 (m, 4H), 3.73 (br s, |
| | acid | | 2H), 2.63 (s, 3H), 2.43-2.26 (m, |
| | | | 2H), 2.24-2.09 (m, 2H), 2.02-1.91 |
| | | | (m, 2H), 0.97 (t, J = 7.1 Hz, 3H). |
| 283 | 2-[2-(3,4-difluoro-2- | 473.07, | ¹H NMR (400 MHz, DMSO-d₆) δ |
| | methoxy-phenoxy)-5- | 474.1 | 17.23 (s, 1H), 13.07 (s, 1H), 7.85 (d, |
| | fluoro-4- | | J = 10.5 Hz, 1H), 7.25 (d, J = 5.7 |
| | (trifluoromethyl)phenyl]-6- | | Hz, 1H), 7.18 (q, J = 9.4 Hz, 1H), |
| | methyl-4-oxo-1H-pyridine- | | 6.83 (ddd, J = 9.5, 5.0, 2.3 Hz, 1H), |
| | 3-carboxylic acid | | 6.73 (s, 1H), 3.79 (d, J = 1.1 Hz, |
| | | | 3H), 2.38 (s, 3H). |

### Compound 284

### 2-[2-(4,4-difluoroazepan-1-yl)-5-methyl-6-(trifluoromethyl)-3-pyridyl]-5,6-dimethyl-4-oxo-1H-pyridine-3-carboxylic acid

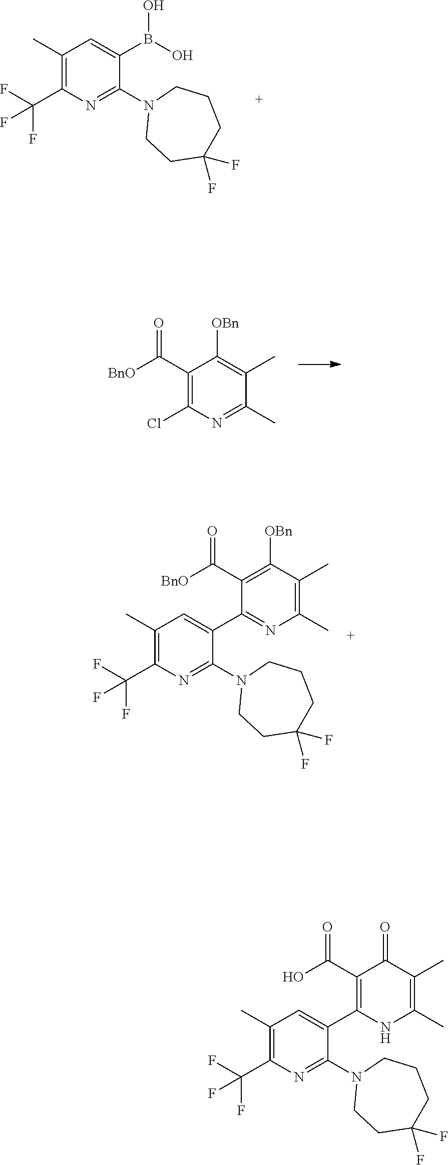 [SMILES: Cc1cc(B(O)O)c(N2CCCC(F)(F)CC2)nc1C(F)(F)F ; Cc1nc(Cl)c(C(=O)OCc2ccccc2)c(OCc2ccccc2)c1C ; Cc1cc(-c2nc(C)c(C)c(OCc3ccccc3)c2C(=O)OCc2ccccc2)c(N2CCCC(F)(F)CC2)nc1C(F)(F)F ; Cc1cc(-c2[nH]c(C)c(C)c(=O)c2C(=O)O)c(N2CCCC(F)(F)CC2)nc1C(F)(F)F]

Benzyl 4-benzyloxy-2-[2-(4,4-difluoroazepan-1-yl)-5-methyl-6-(trifluoromethyl)-3-pyridyl]-5,6-dimethyl-pyridine-3-carboxylate was prepared from [2-(4,4-difluoroazepan-1-yl)-5-methyl-6-(trifluoromethyl)-3-pyridyl]boronic acid and benzyl 4-benzyloxy-2-chloro-5,6-dimethyl-pyridine-3-carboxylate using a cross-coupling procedure analogous to that found in Compound 209, step 1. ESI-MS m/z calc. 639.25, found 640.8 (M+1)⁺. Deprotection of the bis-benzyl-protected intermediate using a debenzylation procedure analogous to that found in Compound 209, step 3 provided 2-[2-(4,4-difluoroazepan-1-yl)-5-methyl-6-(trifluoromethyl)-3-pyridyl]-5,6-dimethyl-4-oxo-1H-pyridine-3-carboxylic acid (Compound 284). ¹H NMR (400 MHz, CD₃OD) δ 7.51 (s, 1H), 3.55 (br s, 2H), 3.21 (br s, 2H) overlaps with CD₂HOD, 2.51 (s, 3H), 2.36 (q, J=2.2 Hz, 3H), 2.26-2.16 (m, 2H) overlaps with 2.22 (s, 3H), 2.05-1.90 (m, 2H), 1.82-1.71 (m, 2H). ¹⁹F NMR (376 MHz, CD₃OD) δ −66.95 (s), −91.95 (m). ESI-MS m/z calc. 459.16, found 460.5 (M+1)⁺.

### Example 8

### Compound 285

### 2-[2-(4,4-difluoroazepan-1-yl)-5-methyl-6-(trifluoromethyl)-3-pyridyl]-5,6-dimethyl-4-oxo-1H-pyridine-3-carboxamide

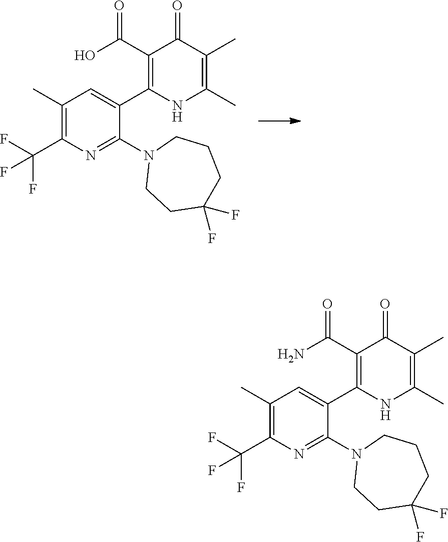 [SMILES: Cc1cc(-c2[nH]c(C)c(C)c(=O)c2C(=O)O)c(N2CCCC(F)(F)CC2)nc1C(F)(F)F ; Cc1cc(-c2[nH]c(C)c(C)c(=O)c2C(N)=O)c(N2CCCC(F)(F)CC2)nc1C(F)(F)F]

A vial containing 2-[2-(4,4-difluoroazepan-1-yl)-5-methyl-6-(trifluoromethyl)-3-pyridyl]-5,6-dimethyl-4-oxo-1H-pyridine-3-carboxylic acid (Compound 284, 3.5 mg, 0.0076 mmol) in DMF (150 μL) was charged with HATU (4 mg, 0.01 mmol), DIPEA (10 μL, 0.057 mmol) and ammonia solution (20 μL of 0.5 M in 1,4-dioxane, 0.01 mmol). The mixture was stirred for 30 min, diluted with 2 mL of DMSO and purified by reverse phase HPLC (10-99% acetonitrile/5 mM HCl over 20 min) to provide 2-[2-(4,4-difluoroazepan-1-yl)-5-methyl-6-(trifluoromethyl)-3-pyridyl]-5,6-dimethyl-4-oxo-1H-pyridine-3-carboxamide (Hydrochloride salt) (285, 1.7 mg, 45%). ¹H NMR (400 MHz, CD₃OD) δ 7.50 (s, 1H), 3.61-3.52 (m, 2H), 3.24 (br m, 2H), 2.48 (s, 3H), 2.35 (q, J=2.2 Hz, 3H), 2.29-2.19 (m, 2H) overlaps with 2.18 (s, 3H), 2.07-1.92 (m, 2H), 1.84-1.73 (m, 2H). ¹⁹F NMR (376 MHz, CD₃OD) δ −66.96, −92.12 (broad). ESI-MS m/z calc. 458.17, found 459.5 (M+1)⁺.

The following compounds were synthesized using a procedure analogous to that in Compound 285 starting from Compound 282, Compound 281, Compound 284, Compound 279, and Compound 284 respectively.

| | | MW & | |
|---|---|---|---|
| Cmpd | | Found | |
| No | Compound Name | [M + H]⁺ | NMR (shifts in ppm) |
| 286 | ethyl 5-(2-amino-2-oxo- | 507.16, | ¹H NMR (400 MHz, DMSO-d₆) δ |
| | ethyl)-2-[2-(3,4-difluoro-2- | 508.64 | 11.99 (br s, 1H), 8.49 (s, 1H), 8.03 (d, |
| | methyl-phenoxy)-3-quinolyl]- | | J = 8.1 Hz, 1H), 7.74 (ddd, J = 8.4, |
| | 6-methyl-4-oxo-1H-pyridine- | | 6.8, 1.5 Hz, 1H), 7.66 (d, J = 8.3 Hz, |
| | 3-carboxylate | | 1H), 7.57 (ddd, J = 8.1, 6.8, 1.3 Hz, |
| | | | 1H), 7.40 (app q, J = 9.4 Hz, 1H), 7.31 |
| | | | (br s, 1H), 7.13-7.04 (m, 1H), 6.84 |
| | | | (br s, 1H), 3.91 (q, J = 7.1 Hz, 2H) |
| | | | overlaps with HOH, 3.34 (s, 2H), 2.32 |
| | | | (s, 3H), 2.06 (d, J = 2.0 Hz, 3H), 0.71 |
| | | | (t, J = 7.1 Hz, 3H). |
| 287 | ethyl 5-(2-amino-2-oxo- | 498.21, | ¹H NMR (400 MHz, DMSO-d₆) δ |
| | ethyl)-2-[2-(4,4- | 499.6 | 11.73 (br s, 1H), 8.08 (s, 1H), 7.80 (d, |
| | difluoroazepan-1-yl)-3- | | J = 8.1 Hz, 1H), 7.71-7.61 (m, 2H), |
| | quinolyl]-6-methyl-4-oxo- | | 7.34 (ddd, J = 8.0, 5.9, 2.1 Hz, 1H), |
| | 1H-pyridine-3-carboxylate | | 7.21 (br s, 1H), 6.80 (br s, 1H), 3.93 |
| | | | (q, J = 7.0 Hz, 2H), 3.61 (s, 2H), 3.55- |
| | | | 3.17 (m, 4H) overlaps with HOH, |
| | | | 2.27 (s, 3H) overlaps with 2.27 (m, |
| | | | 2H), 2.11-1.95 (m, 2H), 1.81-1.74 |
| | | | (m, 2H), 0.83 (t, J = 7.1 Hz, 3H). |
| 288 | 2-[2-(4,4-difluoroazepan-1- | 486.21, | ¹H NMR (400 MHz, CD₃OD) δ 7.44 |
| | yl)-5-methyl-6- | 487.6 | (s, 1H), 3.77-3.61 (m, 1H), 3.56- |
| | (trifluoromethyl)-3-pyridyl]- | | 3.40 (m, 2H), 3.18-3.08 (m, 1H), 2.97 |
| | N,N,5,6-tetramethyl-4-oxo- | | (s, 3H) overlaps with 2.92 (s, 3H), |
| | 1H-pyridine-3-carboxamide | | 2.46 (s, 3H), 2.35 (q, J = 2.1 Hz, 3H) |
| | | | overlaps with 2.33-2.16 (m, 2H) |
| | | | overlaps with 2.14 (s, 3H), 2.08-1.91 |
| | | | (m, 2H), 1.91-1.71 (m, 2H). |
| 289 | 6-[4-tert-butyl-2-(4-fluoro-2- | 466.5; | ¹H NMR (400 MHz, DMSO-d₆) δ |
| | methoxy-phenoxy)-6-methyl- | 467.3 | 11.47 (s, 1H), 7.10 (s, 1H), 7.07-6.98 |
| | phenyl]-N,N,2-trimethyl-4- | | (m, 2H), 6.76 (td, J = 8.5, 2.8 Hz, 1H), |
| | oxo-1H-pyridine-3- | | 6.52 (s, 1H), 5.97 (s, 1H), 3.74 (s, 3H), |
| | carboxamide | | 2.93 (s, 3H), 2.81 (s, 3H), 2.21 (s, 3H), |
| | | | 2.10 (s, 3H), 1.18 (s, 9H). |
| 290 | 2-[2-(4,4-difluoroazepan-1- | 472.45; | ¹H NMR (400 MHz, CD₃OD) δ 7.63 |
| | yl)-5-methyl-6- | 473.6 | (s, 1H), 3.57-3.48 (m, 2H), 3.23- |
| | (trifluoromethyl)-3-pyridyl]- | | 3.07 (br m, 2H), 2.73 (s, 3H), 2.63 (s, |
| | N,5,6-trimethyl-4-oxo-1H- | | 3H), 2.38 (q, J = 2.1 Hz, 3H), 2.30 (s, |
| | pyridine-3-carboxamide | | 3H) overlaps with 2.29-2.20 (m, 2H), |
| | | | 2.05-1.91 (m, 2H), 1.85-1.75 (m, |
| | | | 2H). |
| 291 | 6-[4-tert-butyl-2-(4-fluoro-2- | 438.49; | ¹H NMR (400 MHz, DMSO-d₆) δ |
| | methoxy-phenoxy)-6-methyl- | 439.2 | 11.75 (s, 1H), 9.66 (d, J = 3.9 Hz, 1H), |
| | phenyl]-2-methyl-4-oxo-1H- | | 7.14 (d, J = 3.4 Hz, 1H), 7.10 (s, 1H), |
| | pyridine-3-carboxamide | | 7.09-7.03 (m, 2H), 6.78 (td, J = 8.5, |
| | | | 2.8 Hz, 1H), 6.50 (d, J = 1.0 Hz, 1H), |
| | | | 6.23 (d, J = 1.2 Hz, 1H), 3.73 (s, 3H), |
| | | | 2.61 (s, 3H), 2.20 (s, 3H), 1.18 (s, 9H). |
| 292 | 6-[4-tert-butyl-2-(4-fluoro-2- | 452.52; | ¹H NMR (400 MHz, DMSO-d₆) δ |
| | methoxy-phenoxy)-6-methyl- | 453.3 | 11.78 (s, 1H), 10.20-10.12 (m, 1H), |
| | phenyl]-N,2-dimethyl-4-oxo- | | 7.10 (s, 1H), 7.09-7.03 (m, 2H), 6.77 |
| | 1H-pyridine-3-carboxamide | | (td, J = 8.4, 2.9 Hz, 1H), 6.50 (s, 1H), |
| | | | 6.23 (d, J = 1.5 Hz, 1H), 3.72 (s, 3H), |
| | | | 2.74 (d, J = 4.6 Hz, 3H), 2.61 (s, 3H), |
| | | | 2.20 (s, 3H), 1.18 (s, 9H). |

### Compound 293 and Compound 294

### methyl 2-[2-(3,4-difluoro-2-methyl-phenoxy)-4-methyl-5-(trifluoromethyl)-3-pyridyl]-5,6-dimethyl-4-oxo-1H-pyridine-3-carboxylate and 2-[2-(3,4-difluoro-2-methyl-phenoxy)-4-methyl-5-(trifluoromethyl)-3-pyridyl]-5,6-dimethyl-4-oxo-1H-pyridine-3-carboxamide

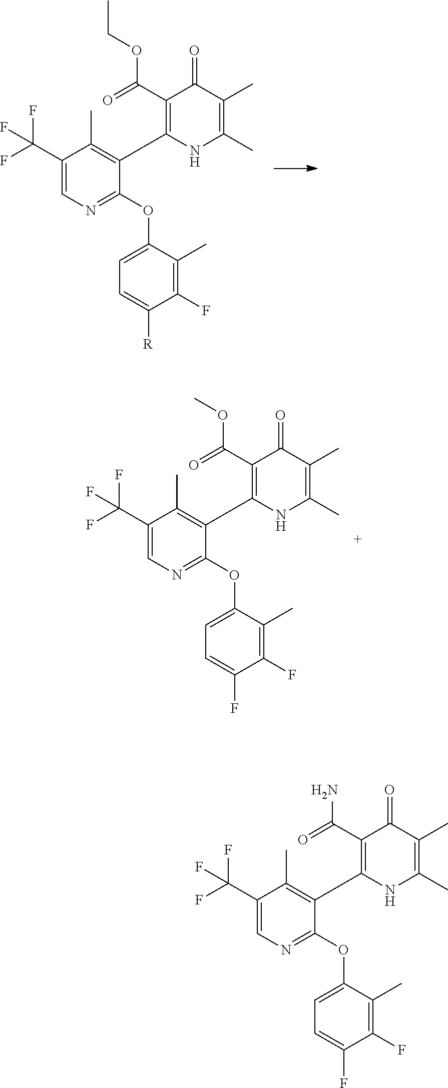

To a solution of ethyl 2-[2-(3,4-difluoro-2-methyl-phenoxy)-4-methyl-5-(trifluoromethyl)-3-pyridyl]-5,6-dimethyl-4-oxo-1H-pyridine-3-carboxylate (Compound 90, 115 mg, 0.232 mmol) in methanol (3 mL) was added ammonia in methanol (7 mL of 7 M, 49 mmol). The resulting mixture was stirred at 60° C. for 16 h. The reaction was allowed to cool to room temperature, then concentrated under reduced pressure. The residue was purified by SFC (ChiralPak IC column, isocratic 88% CO₂/12% methanol with 20 mM NH₃ as modifier) to provide methyl 2-[2-(3,4-difluoro-2-methyl-phenoxy)-4-methyl-5-(trifluoromethyl)-3-pyridyl]-5,6-dimethyl-4-oxo-1H-pyridine-3-carboxylate (Compound 293, 62 mg, 92%), ESI-MS m/z calc. 482.13, found 483.3 (M+1)⁺. ¹H NMR (400 MHz, CD₃OD) δ 8.40 (s, 1H), 7.12 (q, J=9.3 Hz, 1H), 6.90-6.75 (m, 1H), 3.62 (s, 3H), 2.42 (s, 3H), 2.36 (s, 3H), 2.11 (s, 3H), 2.04 (d, J=2.2 Hz, 3H). ¹⁹F NMR (376 MHz, CD₃OD) δ −62.15, −141.41 (d, J=20.3 Hz), −143.70 (d, J=20.4 Hz) ppm; and 2-[2-(3,4-difluoro-2-methyl-phenoxy)-4-methyl-5-(trifluoromethyl)-3-pyridyl]-5,6-dimethyl-4-oxo-1H-pyridine-3-carboxamide (Compound 294, 5.0 mg, 8%), ESI-MS m/z calc. 467.13, found 468.2 (M+1)⁺. ¹H NMR (400 MHz, CD₃OD) δ 8.33 (s, 1H), 7.09 (q, J=9.3 Hz, 1H), 6.83 (m, 1H), 2.42 (s, 3H), 2.34-2.24 (m, 3H), 2.11 (s. 3H), 2.03 (d, J=2.2 Hz, 3H). ¹⁹F NMR (376 MHz, CD₃OD) δ −61.98, −141.61 (d, J=20.7 Hz), −144.04 (d, J=20.4 Hz).

The following compounds were synthesized from the corresponding ethyl ester using a procedure analogous to that in the synthesis of Compound 293 and Compound 294.

| | | MW & | |
|---|---|---|---|
| Cmpd | | Found | |
| No | Compound Name | [M + H]⁻ | NMR (shifts in ppm) |
| 295 | methyl 2-[6-(3,3- | 498.49; | ¹H NMR (400 MHz, CD₃OD) δ 8.55 |
| | difluorocyclobutyl)-4-[4- | 499.3 | (s, 1H), 7.55 (s, 1H), 3.78-3.67 (m, |
| | (trifluoromethyl)cyclohexyl]- | | 1H), 3.64 (s, 3H), 3.15-2.84 (m, |
| | 3-pyridyl]-5,6-dimethyl-4- | | 4H), 2.65 (s, 3H), 2.58-2.46 (m, |
| | oxo-1H-pyridine-3- | | 1H), 2.47-2.36 (m, 1H), 2.33 (s, |
| | carboxylate | | 3H), 2.11-2.02 (m, 2H), 1.84-1.58 |
| | | | (m, 6H). |
| 296 | methyl 6-[6-(3,3- | 484.46; | ¹H NMR (400 MHz, CD₃OD) δ 8.48 |
| | difluorocyclobutyl)-4-[4- | 485.3 | (s, 1H), 7.34 (s, 1H), 6.38 (s, 1H), |
| | (trifluoromethyl)cyclohexyl]- | | 3.90 (s, 3H), 3.64-3.51 (m, 1H), |
| | 3-pyridyl]-2-methyl-4-oxo- | | 2.99-2.86 (m, 4H), 2.81-2.68 (m, |
| | 1H-pyridine-3-carboxylate | | 1H), 2.51-2.34 (m, 4H), 2.10-2.00 |
| | | | (m, 2H), 1.84-1.63 (m, 6H). |
| 297 | methyl 2-[5-(4,4- | 459.41; | ¹H NMR (400 MHz, Acetonitrile-d3) |
| | difluoroazepan-1-yl)-2- | 460 | δ 8.42 (s, 1H), 7.47 (s, 1H), 3.54 (s, |
| | (trifluoromethyl)-4-pyridyl]- | | 3H), 2.38 (s, 2H), 2.16 (s, 6H), 2.09 |
| | 5,6-dimethyl-4-oxo-1H- | | (s, 3H), 1.96 (s, 3H), 1.75 (m, 2H). |
| | pyridine-3-carboxylate | | |
| 298 | methyl 2-[2-(4,4- | 459.41; | ¹H NMR (400 MHz, CD₃OD) δ 8.58- |
| | difluoroazepan-1-yl)-5- | 460.2 | 8.49 (m, 1H), 7.82 (d, J = 2.4 Hz, |
| | (trifluoromethyl)-3-pyridyl]- | | 1H), 3.70-3.61 (m, 5H), 2.51 (s, |
| | 5,6-dimethyl-4-oxo-1H- | | 3H), 2.39-2.23 (m, 2H), 2.18 (s, |
| | pyridine-3-carboxylate | | 3H), 2.05-1.90 (m, 2H), 1.90-1.76 |
| | | | (m, 2H). 2H of azepane ring |
| | | | obscured by solvent peak. |
| 299 | methyl 2-[2-(3,4-difluoro-2- | 468.37; | ¹H NMR (400 MHz, DMSO-d₆) δ |
| | methyl-phenoxy)-5- | 469.4 | 11.89 (s, 1H), 8.67 (d, J = 2.4 Hz, |
| | (trifluoromethyl)-3-pyridyl]- | | 1H), 8.35 (d, J = 2.5 Hz, 1H), 7.38 |
| | 5,6-dimethyl-4-oxo-1H- | | (q, J = 9.4 Hz, 1H), 7.07-6.90 (m, |
| | pyridine-3-carboxylate | | 1H), 3.51 (s, 3H), 2.31 (s, 3H), 2.01 |
| | | | (d, J = 2.1 Hz, 3H), 1.93 (s, 3H). |
| 300 | methyl 2-[2-(3,4-difluoro-2- | 482.4; | ¹H NMR (400 MHz, CD₃OD) δ 7.93 |
| | methyl-phenoxy)-5-methyl- | 483.3 | (s, 1H), 7.12 (q, J = 9.3 Hz, 1H), |
| | 6-(trifluoromethyl)-3- | | 7.00-6.77 (m, 1H), 3.64 (s, 3H), |
| | pyridyl]-5,6-dimethyl-4-oxo- | | 2.48 (q, J = 2.2 Hz, 3H), 2.45 (s, |
| | 1H-pyridine-3-carboxylate | | 3H), 2.12 (s, 3H), 2.03 (d, J = 2.2 |
| | | | Hz, 3H). |
| 301 | methyl 2-[5-(3,4-difluoro-2- | 484.37; | ¹H NMR (500 MHz, DMSO-d₆) δ |
| | methoxy-phenoxy)-2- | 485.3 | 11.73 (s, 1H), 8.35 (s, 1H), 8.07 (s, |
| | (trifluoromethyl)-4-pyridyl]- | | 1H), 7.31-7.19 (m, 1H), 7.04-6.86 |
| | 5,6-dimethyl-4-oxo-1H- | | (m, 1H), 3.81 (s, 3H), 3.51 (s, 3H), |
| | pyridine-3-carboxylate | | 2.28 (s, 3H), 1.89 (s, 3H). |
| 302 | methyl 2-[2-(3,4-difluoro-2- | 501.38; | ¹H NMR (500 MHz, DMSO-d₆) δ |
| | methoxy-phenoxy)-5-fluoro- | 502.45 | 11.66 (s, 1H), 7.75 (s, 1H), 7.23- |
| | 4-(trifluoromethyl)phenyl]- | | 7.18 (m, 2H), 6.86 (s, 1H), 3.79 (s, |
| | 5,6-dimethyl-4-oxo-1H- | | 3H), 3.52 (s, 3H), 2.26 (s, 3H), 1.88 |
| | pyridine-3-carboxylate | | (s, 3H). |
| 255 | 2-[2-(3,4-difluoro-2-methyl- | 453.36; | ¹H NMR (400 MHz, CD₃OD) δ 8.46- |
| | phenoxy)-5- | 454.2 | 8.35 (m, 1H), 8.15 (d, J = 2.4 Hz, |
| | (trifluoromethyl)-3-pyridyl]- | | 1H), 7.11 (q, J = 9.3 Hz, |
| | 5,6-dimethyl-4-oxo-1H- | | 1H), 6.98-6.81 (m, 1H), 2.46 (s, |
| | pyridine-3-carboxamide | | 3H), 2.14 (s, 3H), 2.06 (d, J = 2.2 |
| | | | Hz, 3H). |
| 304 | 2-[2-(3,4-difluoro-2-methyl- | 467.39; | ¹H NMR (400 MHz, CD₃OD) δ 7.79 |
| | phenoxy)-5-methyl-6- | 468.2 | (s, 1H), 7.06 (q, J = 9.3 Hz, 1H), |
| | (trifluoromethyl)-3-pyridyl]- | | 6.93-6.84 (m, 1H), 2.46 (q, J = 2.1 |
| | 5,6-dimethyl-4-oxo-1H- | | Hz, 3H), 2.42 (s, 3H), 2.10 (s, 3H), |
| | pyridine-3-carboxamide | | 2.05 (d, J = 2.3 Hz, 3H). |
| 305 | methyl 6-[5-(3,4-difluoro-2- | 484.10; | ¹H NMR (500 MHz, DMSO-d₆) δ |
| | methoxy-phenoxy)-2- | 485.4 | 11.65 (s, 1H), 8.41 (s, 1H), 8.17 (s, |
| | (trifluoromethyl)-4-pyridyl]- | | 1H), 7.28-7.18 (m, 1H), 7.11-7.04 |
| | 2,5-dimethyl-4-oxo-1H- | | (m, 1H), 3.81 (d, J = 1.4 Hz, 3H), |
| | pyridine-3-carboxylate | | 3.74 (s, 3H), 2.22 (s, 3H), 1.72 (s, |
| | | | 3H). |
| 306 | methyl 6-[6-(3,3- | 498.49; | ¹H NMR (400 MHz, DMSO-d₆) δ |
| | difluorocyclobutyl)-4-[4- | 499.3 | 8.53-8.29 (m, 1H), 7.38 (s, 1H), |
| | (trifluoromethyl)cyclohexyl]- | | 3.75 (s, 3H), 3.70-3.60 (m, 1H), |
| | 3-pyridyl]-2,5-dimethyl-4- | | 3.05-2.72 (m, 4H), 2.48-2.35 (m, |
| | oxo-1H-pyridine-3- | | 2H), 2.20 (s, 3H), 1.99-1.83 (m, |
| | carboxylate | | 2H), 1.78-1.45 (m, 9H). |
| 307 | 6-[6-(3,3- | 483.47; | ¹H NMR (400 MHz, DMSO-d₆) δ |
| | difluorocyclobutyl)-4-[4- | 484.3 | 11.69 (s, 1H), 9.77 (s, 1H), 8.46 (s, |
| | (trifluoromethyl)cyclohexyl]- | | 1H), 7.39 (s, 1H), 7.18 (s, 1H), 3.74- |
| | 3-pyridyl]-2,5-dimethyl-4- | | 3.54 (m, 1H), 3.08-2.71 (m, 4H), |
| | oxo-1H-pyridine-3- | | 2.59 (s, 3H), 2.47-2.35 (m, 2H), |
| | carboxamide | | 2.00-1.82 (m, 2H), 1.79-1.42 (m, |
| | | | 9H) |
| 308 | methyl 6-[5-[2-methoxy-4- | 532.39; | ¹H NMR (400 MHz, DMSO-d₆) δ |
| | (trifluoromethoxy)phenoxy]- | 533.1 | 11.60 (s, 1H), 8.30 (s, 1H), 8.14 (s, |
| | 2-(trifluoromethyl)-4- | | 1H), 7.29 (d, J = 8.9 Hz, 1H), 7.22 |
| | pyridyl]-2,5-dimethyl-4-oxo- | | (s, 1H), 6.99 (d, J = 9.7 Hz, 1H), |
| | 1H-pyridine-3-carboxylate | | 3.77 (s, 3H), 3.75 (s, 3H), 2.22 (s, |
| | | | 3H), 1.71 (s, 3H) |

### Compound 1

### methyl 6-[5-[2-methoxy-4-(trifluoromethoxy)phenoxy]-3-methyl-2-(trifluoromethyl)-4-pyridyl]-2-methyl-4-oxo-1H-pyridine-3-carboxylate

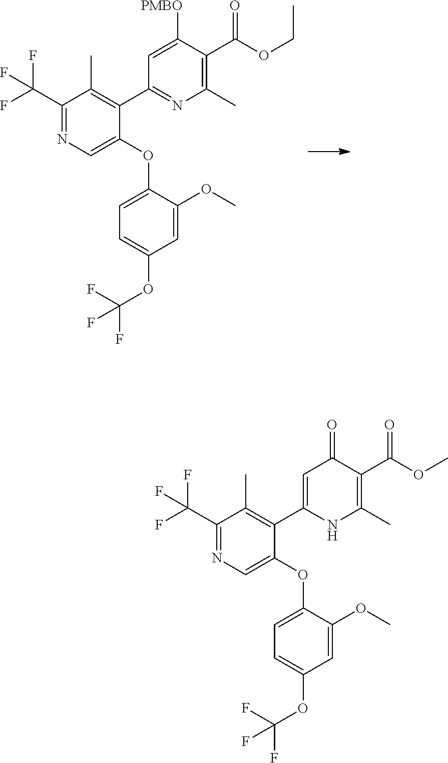

Sodium methoxide in methanol (0.86 mL of 25% w/v, 3.98 mmol) was added to a solution of ethyl 4-[(4-methoxyphenyl)methoxy]-6-[5-[2-methoxy-4-(trifluoromethoxy)phenoxy]-3-methyl-2-(trifluoromethyl)-4-pyridyl]-2-methyl-pyridine-3-carboxylate (65 mg, 0.089 mmol) in 2-MeTHF (1 mL) and the reaction mixture was stirred at room temperature for 2.5 h. The reaction mixture was partitioned between water and ethyl acetate and the aqueous was extracted with ethyl acetate. The combined organics were washed with brine, dried over magnesium sulfate, filtered and concentrated under reduced pressure. The residue was dissolved in DCM (0.5 mL) and TFA (251.60 mg, 0.17 mL, 2.2066 mmol) was added dropwise and the reaction mixture was stirred at room temperature for 1 hour and then quenched with saturated aqueous sodium bicarbonate and extracted with DCM. The organic layer was dried over magnesium sulfate, filtered and concentrated under reduced pressure. Purification by reverse phase chromatography (C18, 30-80% acetonitrile/water, each with 0.1% formic acid) provided methyl 6-[5-[2-methoxy-4-(trifluoromethoxy)phenoxy]-3-methyl-2-(trifluoromethyl)-4-pyridyl]-2-methyl-4-oxo-1H-pyridine-3-carboxylate (22 mg, 46%) as a white solid. ¹H NMR (400 MHz, CD₃OD) δ 7.99 (s, 1H), 7.24 (d, J=8.8 Hz, 1H), 7.10-7.09 (m, 1H), 6.95 (d, J=8.7 Hz, 1H), 6.54 (s, 1H), 3.92 (s, 3H), 3.82 (s, 3H), 2.44 (s, 3H), 2.40 (s, 3H). ¹⁹F NMR (376 MHz, CDCl₃) δ −58.1 (s, 3F), −64.0 (s, 3F). ESI-MS m/z calc. 532.11, found 533.12 (M+1)⁺.

The following compound was prepared from ethyl 6-[5-(3,4-difluoro-2-methoxy-phenoxy)-3-methyl-2-(trifluoromethyl)-4-pyridyl]-4-[(4-methoxyphenyl)methoxy]-2-methyl-pyridine-3-carboxylate using a procedure analogous to that found in Compound 1.

| | | MW & | |
|---|---|---|---|
| Cmpd | | Found | |
| No | Compound Name | [M + H]⁺ | NMR (shifts in ppm) |
| 309 | methyl 6-[5-(3,4-difluoro-2- | 484.11, | ¹H NMR (400 MHz, CDCl₃) δ 12.01 |
| | methoxy-phenoxy)-3- | 485.03 | (d, J = 9.9 Hz, 1H), 7.98 (s, 1H), 6.94 |
| | methyl-2-(trifluoromethyl)- | | (s, 1H), 6.90-6.84 (m, 1H), 6.79 |
| | 4-pyridyl]-2-methyl-4-oxo- | | (ddd, J = 9.2, 5.0, 2.2 Hz, 1H), 4.07 |
| | 1H-pyridine-3-carboxylate | | (s, 3H), 3.88 (d, J = 1.8 Hz, 3H), 2.84 |
| | | | (s, 3H), 2.32 (d, J = 1.6 Hz, 3H) |

### Compound 310

### 6-[2-(3,4-difluoro-2-methyl-phenoxy)-4-methyl-5-(trifluoromethyl)-3-pyridyl]-4-oxo-1H-pyridine-3-carboxamide

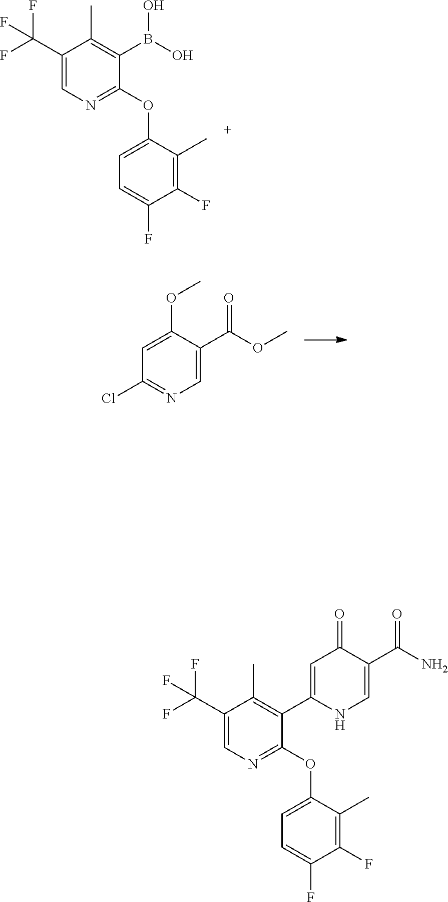 [SMILES: Cc1c(Oc2ncc(C(F)(F)F)c(C)c2B(O)O)ccc(F)c1F ; COC(=O)c1cnc(Cl)cc1OC ; Cc1c(Oc2ncc(C(F)(F)F)c(C)c2-c2cc(=O)c(C(N)=O)c[nH]2)ccc(F)c1F]

A 1,4-dioxane (4.5 mL) solution of methyl 6-chloro-4-methoxy-pyridine-3-carboxylate (34 mg, 0.17 mmol), [2-(3,4-difluoro-2-methyl-phenoxy)-4-methyl-5-(trifluoromethyl)-3-pyridyl]boronic acid (50 mg, 0.14 mmol), PdCl₂(dtbpf) (4.7 mg, 0.0072 mmol), and potassium phosphate (440 μL of 1 M in water, 0.44 mmol) was sparged with nitrogen for 3 min and then stirred at room temperature for 2 h. Purification by reverse phase HPLC (C18, 1-100% acetonitrile/5 mM HCl over 15 min) provided the ester cross-coupling product, which was treated with ammonia (1000 μL of 7 M in methanol, 7 mmol) and stirred at 60° C. for 16 h and then concentrated in vacuo. Purification by reverse phase HPLC (C18, 1-00% acetonitrile/5 mM HCl over 15 min) and concentration provided a mixture of 6-[2-(3,4-difluoro-2-methyl-phenoxy)-4-methyl-5-(trifluoromethyl)-3-pyridyl]-4-methoxy-pyridine-3-carboxamide and 6-[2-(3,4-difluoro-2-methyl-phenoxy)-4-methyl-5-(trifluoromethyl)-3-pyridyl]-4-oxo-1H-pyridine-3-carboxamide. Additional HPLC purification provided 6-[2-(3,4-difluoro-2-methyl-phenoxy)-4-methyl-5-(trifluoromethyl)-3-pyridyl]-4-oxo-1H-pyridine-3-carboxamide (310, 0.8 mg, 1%). ESI-MS m/z calc. 439.10, found 440.2 (M+1)⁺.

### Example 9

### Compound 311

### methyl 6-[2-(3,4-difluoro-2-methoxy-phenoxy)-5-fluoro-4-(trifluoromethyl)phenyl]-2,5-dimethyl-4-oxo-1H-pyridine-3-carboxylate

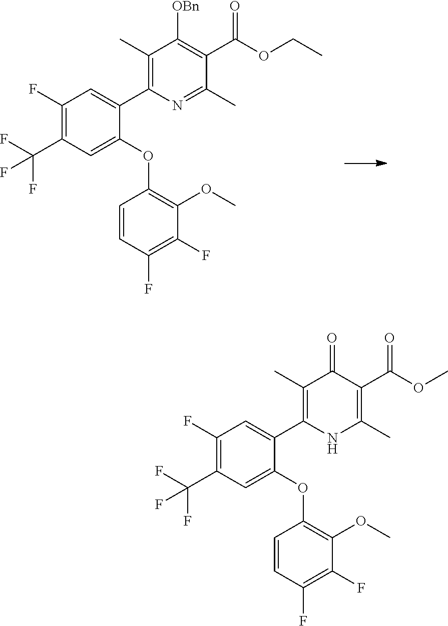 [SMILES: CCOC(=O)c1c(C)nc(-c2cc(F)c(C(F)(F)F)cc2Oc2ccc(F)c(F)c2OC)c(C)c1OCc1ccccc1 ; COC(=O)c1c(C)[nH]c(-c2cc(F)c(C(F)(F)F)cc2Oc2ccc(F)c(F)c2OC)c(C)c1=O]

To a solution of ethyl 4-benzyloxy-6-[2-(3,4-difluoro-2-methoxy-phenoxy)-5-fluoro-4-(trifluoromethyl)phenyl]-2,5-dimethyl-pyridine-3-carboxylate (47 mg, 0.078 mmol) in methanol (2 mL) was added sulfuric acid (20 μL, 0.38 mmol) and the reaction mixture was heated at 60° C. for 4 days.

The reaction mixture was partitioned between ethyl acetate (30 mL) and water (30 mL), and the aqueous layer was extracted with ethyl acetate (2×30 mL). The combined organic extracts were dried over magnesium sulfate, filtered and concentrated in vacuo. Purification by silica gel chromatography (0-20% ethyl acetate/heptane provided methyl 6-[2-(3,4-difluoro-2-methoxy-phenoxy)-5-fluoro-4-(trifluoromethyl)phenyl]-2,5-dimethyl-4-oxo-1H-pyridine-3-carboxylate (311, 8 mg, 21%). ¹H NMR (400 MHz, DMSO-d₆) δ 11.59 (s, 1H), 7.85-7.78 (m, 1H), 7.28 (d, J=5.9 Hz, 1H), 7.22-7.10 (m, 1H), 6.92-6.85 (m, 1H), 3.78 (d, J=1.3 Hz, 3H), 3.72 (s, 3H), 2.18 (s, 3H), 1.70 (s, 3H). ESI-MS m/z calc. 501.10, found 502.4 (M+1)⁺; 500.4 (M−1)⁻.

### Example 10

### Compound 312

### ethyl 2-[2-(3,4-difluoro-2-methyl-phenoxy)-3-quinolyl]-5-ethynyl-6-methyl-4-oxo-1H-pyridine-3-carboxylate

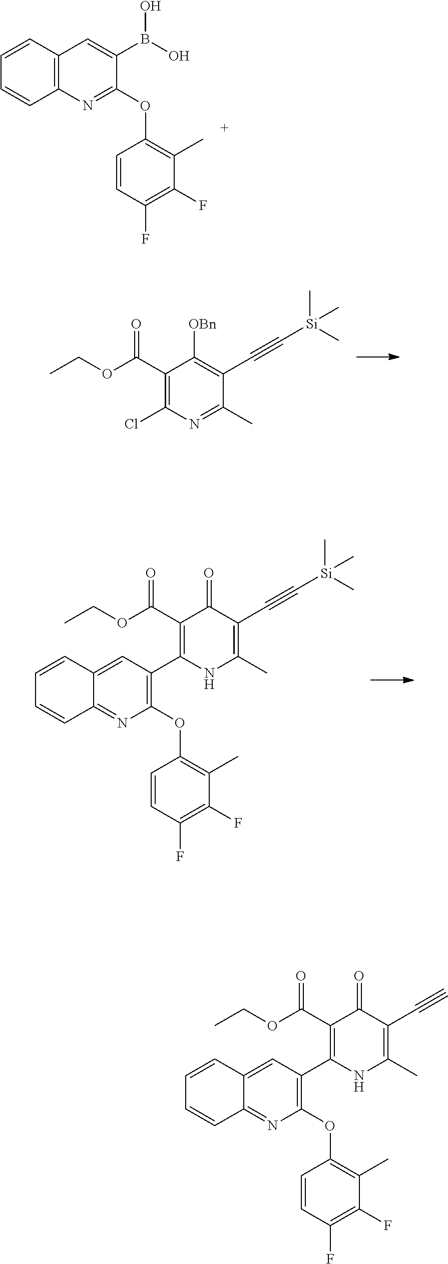 [SMILES: Cc1c(Oc2nc3ccccc3cc2B(O)O)ccc(F)c1F ; CCOC(=O)c1c(Cl)nc(C)c(C#C[Si](C)(C)C)c1OCc1ccccc1 ; CCOC(=O)c1c(-c2cc3ccccc3nc2Oc2ccc(F)c(F)c2C)[nH]c(C)c(C#C[Si](C)(C)C)c1=O ; C#Cc1c(C)[nH]c(-c2cc3ccccc3nc2Oc2ccc(F)c(F)c2C)c(C(=O)OCC)c1=O]

### Step 1: ethyl 2-[2-(3,4-difluoro-2-methyl-phenoxy)-3-quinolyl]-6-methyl-4-oxo-5-(2-trimethylsilylethynyl)-1H-pyridine-3-carboxylate

A mixture of [2-(3,4-difluoro-2-methyl-phenoxy)-3-quinolyl]boronic acid (23.4 mg, 0.0689 mmol), ethyl 4-benzyloxy-2-chloro-6-methyl-5-(2-trimethylsilylethynyl)pyridine-3-carboxylate (21.3 mg, 0.0530 mmol), SPhos Pd G3 (9.5 mg, 0.012 mmol) and potassium phosphate (51 mg, 0.24 mmol) in 1,4-dioxane (1 mL) and water (250 L) was degassed for 5 min. The vial was scaled and stirred at 75° C. for 1 h under nitrogen. The mixture was cooled, diluted with ethyl acetate and washed with saturated aqueous ammonium chloride and brine. The organic layer was dried over magnesium sulfate, filtered, and concentrated under reduced pressure. Purification by silica gel chromatography (0-70% of ethyl acetate/hexanes over 20 min) provided ethyl 2-[2-(3,4-difluoro-2-methyl-phenoxy)-3-quinolyl]-6-methyl-4-oxo-5-(2-trimethylsilylethynyl)-1H-pyridine-3-carboxylate. ESI-MS m/z calc. 546.18, found 547.3 (M+1)⁺.

### Step 2: ethyl 2-[2-(3,4-difluoro-2-methyl-phenoxy)-3-quinolyl]-5-ethynyl-6-methyl-4-oxo-1H-pyridine-3-carboxylate, Compound 312

Ethyl 2-[2-(3,4-difluoro-2-methyl-phenoxy)-3-quinolyl]-6-methyl-4-oxo-5-(2-trimethylsilylethynyl)-1H-pyridine-3-carboxylate from step 1 was dissolved in methanol (2 mL), followed by the addition of potassium carbonate (30 mg, 0.22 mmol). The mixture was stirred at room temperature for 2 h, then diluted with ethyl acetate and washed with a saturated aqueous ammonium chloride and brine. The organic layer was dried over magnesium sulfate, filtered, and concentrated under reduced pressure. Purification by reverse phase HPLC (10-99% acetonitrile/5 mM HCl over 20 min) provided ethyl 2-[2-(3,4-difluoro-2-methyl-phenoxy)-3-quinolyl]-5-ethynyl-6-methyl-4-oxo-1H-pyridine-3-carboxylate (1 mg, 4%). ¹H NMR (400 MHz, CD₃OD) δ 8.41 (s, 1H), 7.94 (d, J=8.1 Hz, 1H), 7.77-7.67 (m, 2H), 7.54 (ddd, J=8.0, 6.2, 1.8 Hz, 1H), 7.17 (app q, J=9.2 Hz, 1H), 7.08-6.98 (m, 1H), 4.02 (q, J=7.2 Hz, 2H) overlaps with 3.97 (s, 1H), 2.58 (s, 3H), 2.10 (d, J=2.2 Hz, 3H), 0.80 (t, J=7.1 Hz, 3H). ¹⁹F NMR (376 MHz, CD₃OD) δ −141.56 (d, J=20.6 Hz), −144.12 (d, J=20.3 Hz). ESI-MS m/z calc. 474.14, found 475.5 (M+1)⁺.

### Example 11

### Compound 313

### 6-[4-tert-butyl-2-(4-fluoro-2-methoxy-phenoxy)-6-methyl-phenyl]-3-(ethoxymethyl)-2-methyl-1H-pyridin-4-one

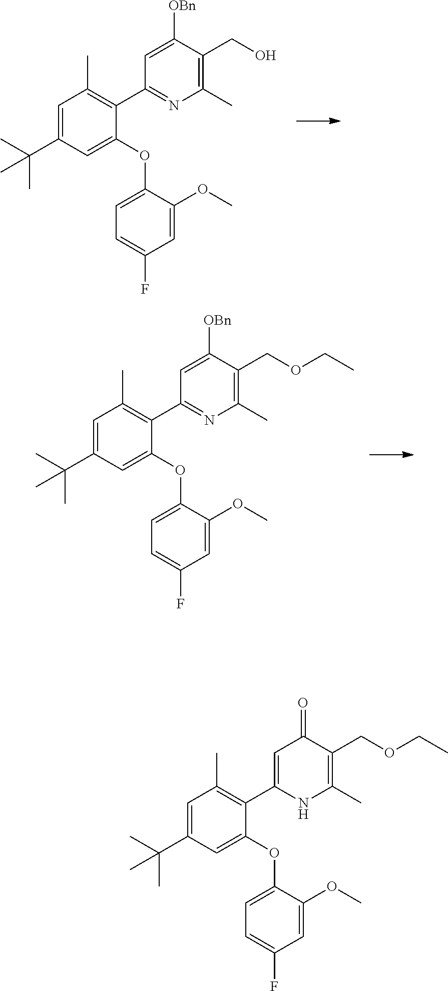

### Step 1: [4-benzyloxy-6-[4-tert-butyl-2-(4-fluoro-2-methoxy-phenoxy)-6-methyl-phenyl]-2-methyl-3-pyridyl]methanol

To a solution of ethyl 4-benzyloxy-6-[4-tert-butyl-2-(4-fluoro-2-methoxy-phenoxy)-6-methyl-phenyl]-2-methyl-pyridine-3-carboxylate (149 mg, 0.1916 mmol) in tetrahydrofuran (2 mL) at 0° C. was added a lithium aluminum hydride (1 mL of 1 M in THF, 1 mmol). The mixture was stirred at 0° C. for 1.5 h, then quenched by careful addition of a few drops of ethyl acetate. The mixture was diluted with ethyl acetate (70 mL) and washed with 1:1 5% aqueous citric acid/1 M HCl (20 mL), water (10 mL) and brine (10 mL). The organic phase was dried over sodium sulfate, filtered and evaporated under reduced pressure. Purification by silica gel chromatography (0-10% methanol/DCM), followed by reverse phase purification (C18, 5-95% acetonitrile/water, each with 0.1% formic acid) provided [4-benzyloxy-6-[4-tert-butyl-2-(4-fluoro-2-methoxy-phenoxy)-6-methyl-phenyl]-2-methyl-3-pyridyl]methanol (95 mg, 91%). ESI-MS m/z calc. 515.25, found 516.3 (M+1)⁺. ¹H NMR (400 MHz, CDCl₃) δ 7.42-7.30 (m, 5H), 7.02 (s, 1H), 6.97 (s, 1H), 6.79 (dd, J=8.6, 5.7 Hz, 1H), 6.70 (s, 1H), 6.59 (dd, J=10.3, 2.8 Hz, 1H), 6.52 (dt, J=8.4, 2.8 Hz, 1H), 5.08 (s, 2H), 4.79 (s, 2H), 3.72 (s, 3H), 2.59 (s, 3H), 2.19 (s, 3H), 1.23 (s, 9H). ¹⁹F NMR (377 MHz, CDCl₃) δ −117.28 (s, 1F).

### Step 2: 4-benzyloxy-6-[4-tert-butyl-2-(4-fluoro-2-methoxy-phenoxy)-6-methyl-phenyl]-3-(ethoxymethyl)-2-methyl-pyridine

To a solution of [4-benzyloxy-6-[4-tert-butyl-2-(4-fluoro-2-methoxy-phenoxy)-6-methyl-phenyl]-2-methyl-3-pyridyl]methanol (150 mg, 0.280 mmol) in tetrahydrofuran (5 mL) at 0° C. was added sodium hydride (74 mg, 60% dispersion in mineral oil, 1.9 mmol). After 5 min, iodoethane (290 mg, 0.15 mL, 1.9 mmol) was added and the mixture was stirred at room temperature for 19 h. The mixture was diluted with 2-MeTHF (100 mL), washed with water and brine, dried over sodium sulfate, filtered and evaporated under reduced pressure. Purification by silica gel chromatography (0-25% ethyl acetate/heptanes) provided 4-benzyloxy-6-[4-tert-butyl-2-(4-fluoro-2-methoxy-phenoxy)-6-methyl-phenyl]-3-(ethoxymethyl)-2-methyl-pyridine (145 mg, 89%) as a colorless oil. ESI-MS m/z calc. 543.28, found 544.4 (M+1)⁺. ¹H NMR (400 MHz, CDCl₃) δ 7.40-7.28 (m, 5H), 7.02 (s, 1H), 6.92 (s, 1H), 6.76 (dd, J=8.8, 5.8 Hz, 1H), 6.72 (d, J=1.3 Hz, 1H), 6.57 (dd, J=10.2, 2.8 Hz, 1H), 6.50 (dt, J=8.4, 2.9 Hz, 1H), 5.05 (s, 2H), 4.60 (s, 2H), 3.71 (s, 3H), 3.55 (q, J=7.0 Hz, 2H), 2.56 (s, 3H), 2.19 (s, 3H), 1.29-1.19 (m, 12H). ¹⁹F NMR (377 MHz, CDCl₃) δ −117.66 (s, 1F).

### Step 3: 6-[4-tert-butyl-2-(4-fluoro-2-methoxy-phenoxy)-6-methyl-phenyl]-3-(ethoxymethyl)-2-methyl-1H-pyridin-4-one, Compound 313

6-[4-tert-Butyl-2-(4-fluoro-2-methoxy-phenoxy)-6-methyl-phenyl]-3-(ethoxymethyl)-2-methyl-1H-pyridin-4-one was prepared from 4-benzyloxy-6-[4-tert-butyl-2-(4-fluoro-2-methoxy-phenoxy)-6-methyl-phenyl]-3-(ethoxymethyl)-2-methyl-pyridine using a procedure analogous to that found in Compound 209, step 3 with ethyl acetate as the solvent. ESI-MS m/z calc. 453.23, found 454.3 (M+1)⁺. ¹H NMR (400 MHz, DMSO-d₆) δ 13.82 (br s, 1H), 7.16 (s, 1H), 7.12-7.02 (m, 3H), 6.79 (td, J=8.4, 2.9 Hz, 1H), 6.55 (s, 1H), 4.48 (s, 2H), 3.72 (s, 3H), 3.51 (q, J=7.1 Hz, 2H), 2.58 (s, 3H), 2.18 (s, 3H), 1.19 (s, 9H), 1.14 (t, J=7.0 Hz, 3H). ¹⁹F NMR (377 MHz, DMSO-d₆) δ −114.43 (s, 1F).

The following compounds were prepared from the Suzuki cross-coupling between appropriate Intermediate-B and ethyl 4-benzyloxy-2-chloro-5,6-dimethyl-pyridine-3-carboxylate (Intermediate A-15), followed by ester reduction and debenzylation using a procedure analogous to that found in the synthesis of Compound 313.

| | | MW & | |
|---|---|---|---|
| Cmpd | | Found | |
| No | Compound Name | [M + H]⁺ | NMR (shifts in ppm) |
| 314 | 2-[2-(4,4-difluoroazepan-1- | 445.18; | ¹H NMR (400 MHz, CD₃OD) δ 7.69 |
| | yl)-5-methyl-6- | 446.5 | (s, 1H), 4.64 (br s, 2H), 3.58-3.51 (m, |
| | (trifluoromethyl)-3-pyridyl]- | | 2H), 3.23-2.88 (br m, 3H), 2.65 (s, |
| | 3-(hydroxymethyl)-5,6- | | 3H), 2.40 (q, J = 2.1 Hz, 3H), 2.32 (s, |
| | dimethyl-1H-pyridin-4-one | | 3H) overlaps with 2.29-2.18 (m, 2H), |
| | | | 1.97 (m, 2H), 1.79 (m, 2H). |
| 315 | 2-[2-(3,4-difluoro-2- | 473.11; | ¹H NMR (500 MHz, DMSO-d₆) δ |
| | methoxy-phenoxy)-5-fluoro- | 474.4 | 11.37 (s, 1H), 7.77 (d, J = 10.2 Hz, |
| | 4-(trifluoromethyl)phenyl]-3- | | 1H), 7.23 (d, J = 5.9 Hz, 1H), 7.21- |
| | (hydroxymethyl)-5,6- | | 7.11 (m, 1H), 6.95-6.88 (m, 1H), |
| | dimethyl-1H-pyridin-4-one | | 4.91 (s, 1H), 4.29 (s, 1H), 4.01 (s, 1H), |
| | | | 3.78 (d, J = 1.2 Hz, 3H), 2.24 (s, 3H), |
| | | | 1.88 (s, 3H). |

### Compound 316

### 2-[5-fluoro-2-[2-methoxy-4-(trifluoromethoxy)phenoxy]-4-(trifluoromethyl)phenyl]-6-methyl-1H-pyridin-4-one

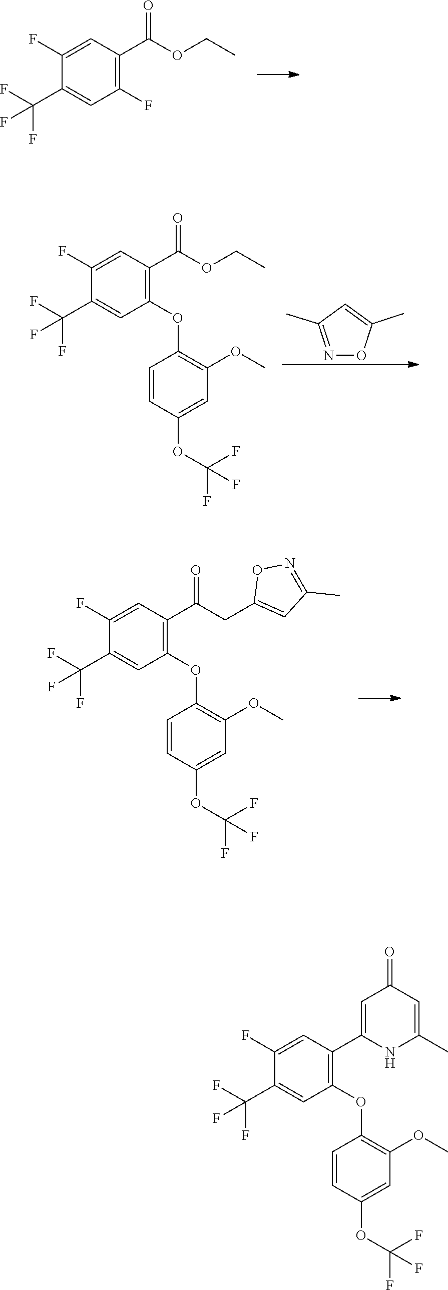

### Step 1: ethyl 5-fluoro-2-[2-methoxy-4-(trifluoromethoxy)phenoxy]-4-(trifluoromethyl)benzoate

A solution of ethyl 2,5-difluoro-4-(trifluoromethyl)benzoate (2.425 g, 9.541 mmol), 2-methoxy-4-(trifluoromethoxy)phenol (2.191 g, 10.53 mmol) and cesium carbonate (3.736 g, 11.47 mmol) in acetonitrile (25 mL) was purged with nitrogen and the mixture heated at 80° C. for 20 h. The reaction mixture was filtered, the filter cake washed with acetonitrile and the filtrate concentrated in vacuo. The crude residue partitioned between with ethyl acetate (100 mL) and water (20 mL) and the layers separated. The aqueous layer was extracted with additional ethyl acetate (2×75 mL). The combined organic extracts were dried over magnesium sulfate, filtered and concentrated in vacuo. Purification by silica gel chromatography (0-10% ethyl acetate/heptane) provided ethyl 5-fluoro-2-[2-methoxy-4-(trifluoromethoxy)phenoxy]-4-(trifluoromethyl)benzoate (1.702 g, 40%). ¹H NMR (400 MHz, CDCl₃) δ 7.71 (d, J=9.9 Hz, 1H), 7.06 (d, J=5.7 Hz, 1H), 6.87 (d, J=8.8 Hz, 2H), 6.82-6.77 (m, 1H), 4.33 (q, J=7.1 Hz, 2H), 3.84 (s, 3H), 1.29 (t, J=7.1 Hz, 3H). ESI-MS m/z calc. 442.07, found 441.4 (M−1)⁻.

### Step 2: 1-[5-fluoro-2-[2-methoxy-4-(trifluoromethoxy)phenoxy]-4-(trifluoromethyl)phenyl]-2-(3-methylisoxazol-5-yl)ethanone

To a solution of 3,5-dimethylisoxazole (520 μL, 5.30 mmol) in THF (15 mL) at −78° C. was added LDA (2.8 mL of 2 M, 5.600 mmol) and the reaction mixture was stirred at this temperature for 1 h. A solution of ethyl 5-fluoro-2-[2-methoxy-4-(trifluoromethoxy)phenoxy]-4-(trifluoromethyl)benzoate (782 mg, 1.77 mmol) in THF (4 mL) was added at −78° C. The mixture was stirred at this temperature for 1 h, then warmed to room temperature and stirred for 1 h. The mixture was quenched with saturated ammonium chloride solution (40 mL) and extracted with ethyl acetate (2×40 mL). The combined organic extracts were washed with brine (30 mL), dried over magnesium sulfate, filtered and concentrated in vacuo. Purification by silica gel chromatography (0-20% ethyl acetate/heptane) provided 1-[5-fluoro-2-[2-methoxy-4-(trifluoromethoxy)phenoxy]-4-(trifluoromethyl)phenyl]-2-(3-methylisoxazol-5-yl)ethanone (240 mg, 28%). ¹H NMR (500 MHz, CDCl₃) δ 7.66 (dd, J=10.0, 0.8 Hz, 1H), 7.14 (dd, J=8.2, 0.9 Hz, 1H), 6.97-6.84 (m, 3H), 6.16 (s, 1H), 4.59 (d, J=0.7 Hz, 2H), 3.79 (s, 3H), 2.29 (s, 3H). ESI-MS m/z calc. 493.08, found 494.4 (M+1)⁺; 492.4 (M−1)⁻.

### Step 3: 2-[5-fluoro-2-[2-methoxy-4-(trifluoromethoxy)phenoxy]-4-(trifluoromethyl)phenyl]-6-methyl-1H-pyridin-4-one, Compound 316

To a solution of 1-[5-fluoro-2-[2-methoxy-4-(trifluoromethoxy)phenoxy]-4-(trifluoromethyl)phenyl]-2-(3-methylisoxazol-5-yl)ethanone (240 mg, 0.487 mmol), molybdenum hexacarbonyl (158 mg, 0.599 mmol) and water (270 μL, 14.99 mmol) in acetonitrile (4 mL) was heated to 80° C. for 3 h. The reaction mixture was concentrated in vacuo. Purification by silica gel chromatography (0-70% ethyl acetate/heptane) followed by reverse phase purification (C18, acetonitrile/water gradient with ammonia modifier) provided 2-[5-fluoro-2-[2-methoxy-4-(trifluoromethoxy)phenoxy]-4-(trifluoromethyl)phenyl]-6-methyl-1H-pyridin-4-one (316, 56 mg, 24%). ¹H NMR (500 MHz, DMSO-d₆) δ 10.67 (s, 1H), 7.92 (d, J=11.5 Hz, 1H), 7.31 (s, 1H), 7.24-7.15 (m, 2H), 7.04-6.94 (m, 2H), 6.65 (s, 1H), 3.79 (s, 3H), 2.40 (s, 3H). ESI-MS m/z calc. 477.08, found 478.4 (M+1)⁺; 476.4 (M−1)⁻.

### Compound 317

### 2-[3-(3,4-difluoro-2-methyl-phenoxy)quinoxalin-2-yl]-6-methyl-1H-pyridin-4-one

### Step 1: 2-chloro-3-(3,4-difluoro-2-methyl-phenoxy)quinoxaline

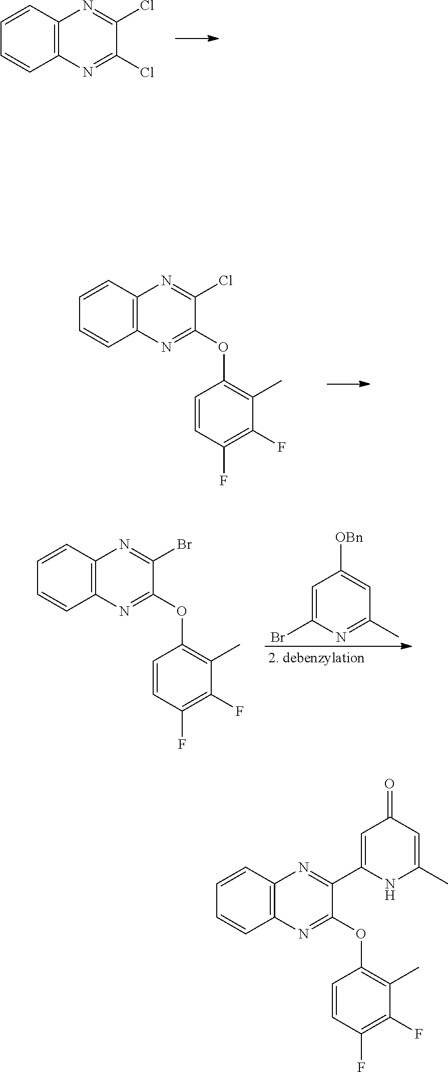

2,3-Dichloroquinoxaline (500 mg, 2.51 mmol), 3,4-difluoro-2-methyl-phenol (366 mg, 2.54 mmol) and cesium carbonate (870 mg, 2.67 mmol) were combined in DMF (5 mL) and heated at 80° C. for 90 min. The mixture was cooled and partitioned between ethyl acetate and water. The organic layer was washed with brine, dried over sodium sulfate and filtered. Purification by silica gel chromatography (0-20% ethyl acetate/hexane) provided 2-chloro-3-(3,4-difluoro-2-methyl-phenoxy)quinoxaline (760 mg, 99%). ESI-MS m/z calc. 306.03, found 307.1 (M+1)⁺.

### Step 2: 2-bromo-3-(3,4-difluoro-2-methyl-phenoxy)quinoxaline

A mixture of bromo(trimethyl)silane (100 mg, 86 μL, 0.65 mmol) and 2-chloro-3-(3,4-difluoro-2-methyl-phenoxy)quinoxaline (100 mg, 0.326 mmol) in propanenitrile (8 mL) was microwaved at 150° C. for 90 min and then diluted with ethyl acetate (15 mL) and saturated aqueous sodium bicarbonate solution (15 mL). The organic layer was separated, washed with water (10 mL) and brine (10 mL), dried over sodium sulfate, filtered, and concentrated in vacuo to provide 2-bromo-3-(3,4-difluoro-2-methyl-phenoxy)quinoxaline (107.8 mg, 46%). ESI-MS m/z calc. 349.99, found 351.0 (M+1)⁺.

### Step 3: 2-[3-(3,4-difluoro-2-methyl-phenoxy)quinoxalin-2-yl]-6-methyl-1H-pyridin-4-one, Compound 317

A solution of 2-bromo-3-(3,4-difluoro-2-methyl-phenoxy)quinoxaline (50 mg, 0.10 mmol) in THF (0.6 mL) was cooled to 0° C. under nitrogen atmosphere and treated with iPrMgCl (400 μL of 1.3 M, 0.52 mmol). The mixture was stirred for 4 h and then treated with ZnCl₂ (700 μL of 0.5 M in THF, 0.35 mmol) and stirred at room temperature for 1 h. In a separate vial, 4-benzyloxy-2-bromo-6-methyl-pyridine (64 mg, 0.23 mmol) and Pd(PPh₃)₄ (26.7 mg, 0.0231 mmol) were dissolved in THF (0.5 mL), heated to 65° C. and sparged with nitrogen for 1 minute. The organozinc reagent solution prepared above was added to the second vial at 65° C. and stirred for 16 h. The mixture was filtered and purified by reverse phase HPLC (C18, 20-80% acetonitrile/5 mM HCl) to provide 2-(4-benzyloxy-6-methyl-2-pyridyl)-3-(3,4-difluoro-2-methyl-phenoxy)quinoxaline (6 mg, 13%). ESI-MS m/z calc. 469.16, found 470.2 (M+1)⁺. The benzyl-protected intermediate was dissolved in ethyl acetate (0.7 mL) and stirred with 10% Pd/C (9.7 mg, 0.0091 mmol) under hydrogen atmosphere for 90 min. Filtration and HPLC purification (C18, 1-99% acetonitrile/5 mM HCl) provided 2-[3-(3,4-difluoro-2-methyl-phenoxy)quinoxalin-2-yl]-6-methyl-1H-pyridin-4-one (317, 1.3 mg, 3%). ¹H NMR (400 MHz, CD₃OD) δ 8.29 (d, 1H), 8.06 (d, J=2.4 Hz, 1H), 7.91-7.75 (m, 3H), 7.26 (q, J=9.2 Hz, 1H), 7.13 (ddd, J=9.1, 4.2, 2.1 Hz, 1H), 7.03 (d, J=2.4 Hz, 1H), 2.77 (s, 3H), 2.16 (d, J=2.2 Hz, 3H). ESI-MS m/z cale. 379.11, found 380.1 (M+1)⁺.

### Compound 318

### 2-[2-(4,4-difluoroazepan-1-yl)-4-(trifluoromethyl)-3-pyridyl]-6-methyl-1H-pyri din-4-one

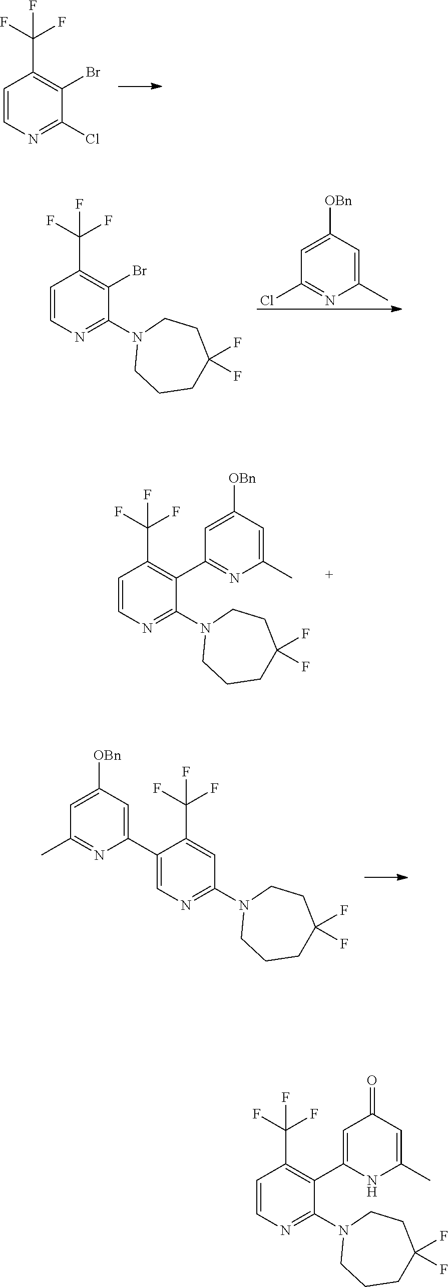 [SMILES: FC(F)(F)c1ccnc(Cl)c1Br ; FC1(F)CCCN(c2nccc(C(F)(F)F)c2Br)CC1 ; Cc1cc(OCc2ccccc2)cc(Cl)n1 ; Cc1cc(OCc2ccccc2)cc(-c2c(C(F)(F)F)ccnc2N2CCCC(F)(F)CC2)n1 ; Cc1cc(OCc2ccccc2)cc(-c2cnc(N3CCCC(F)(F)CC3)cc2C(F)(F)F)n1 ; Cc1cc(=O)cc(-c2c(C(F)(F)F)ccnc2N2CCCC(F)(F)CC2)[nH]1]

### Step 1: 1-[3-bromo-4-(trifluoromethyl)-2-pyridyl]-4,4-difluoro-azepane

A mixture of 3-bromo-2-chloro-4-(trifluoromethyl)pyridine (1.0 g, 3.8 mmol), 4,4-difluoroazepane (780 mg, 5.77 mmol) and DIPEA (1.1 mL, 6.3 mmol) in 1,4-dioxane (10 mL) was heated at 80° C. in a sealed tube for 48 h, followed by heating at 110° C. for 3 days. Additional 4,4-difluoroazepane (260 mg, 1.92 mmol) and DIPEA (670 μL, 3.85 mmol) were added and the reaction stirred for an additional 9 days at 110° C. The mixture was diluted with saturated ammonium chloride and extracted with ethyl acetate (3×). The combined extracts were washed with brine, dried with sodium sulfate, filtered, and concentrated. Purification by silica gel chromatography (0-15% ethyl acetate/hexanes) provided 1-[3-bromo-4-(trifluoromethyl)-2-pyridyl]-4,4-difluoro-azepane (890 mg, 58%) as a clear oil. ¹H NMR (400 MHz, CDCl₃) δ 8.28 (dd, J=4.9, 0.8 Hz, 1H), 7.10 (d, J=4.9 Hz, 1H), 3.68-3.52 (m, 4H), 2.52-2.33 (m, 2H), 2.33-2.13 (m, 2H), 2.06-1.90 (m, 2H). ESI-MS m/z calc. 358.01, found 359.0 (M+1)⁺.

### Step 2: 1-[3-(4-benzyloxy-6-methyl-2-pyridyl)-4-(trifluoromethyl)-2-pyridyl]-4,4-difluoro-azepane

To a solution of 1-[3-bromo-4-(trifluoromethyl)-2-pyridyl]-4,4-difluoro-azepane (278 mg, 0.696 mmol) in THF (500 μL) at −78° C. was added n-BuLi (280 μL of 2.5 M, 0.70 mmol). The mixture was then wanned to −40° C. and allowed to stir for 30 min. ZnCl₂ (1.43 mL of 0.5 M, 0.7 mmol) was added and the reaction was allowed to stir for 10 min at −40° C. before being allowed to warm to room temperature and stirred for 30 min. A separate vial containing a mixture of 4-benzyloxy-2-chloro-6-methyl-pyridine (83 mg, 0.36 mmol), XPhos Pd G3 (30 mg, 0.035 mmol) in THF (800 L) was then sparged with argon for 10 min and heated to 65° C. The organozine reagent was then added dropwise to this mixture and the mixture stirred overnight at 65° C. The reaction was diluted with saturated sodium bicarbonate and extracted with ethyl acetate (3×). The combined organic extracts were dried over sodium sulfate, filtered and concentrated. Purification by silica gel chromatography (0-60% ethyl acetate/DCM) afforded an inseparable mixture of 1-[3-(4-benzyloxy-6-methyl-2-pyridyl)-4-(trifluoromethyl)-2-pyridyl]-4,4-difluoro-azepane and 1-[5-(4-benzyloxy-6-methyl-2-pyridyl)-4-(trifluoromethyl)-2-pyridyl]-4,4-difluoro-azepane (63 mg, 37%). ESI-MS m/z calc. 477.18, found 478.0 (M+1)⁺.

### Step 3: 2-[2-(4,4-difluoroazepan-1-yl)-4-(trifluoromethyl)-3-pyridyl]-6-methyl-1H-pyridin-4-one, Compound 318

The mixture of 1-[3-(4-benzyloxy-6-methyl-2-pyridyl)-4-(trifluoromethyl)-2-pyridyl]-4,4-difluoro-azepane and 1-[5-(4-benzyloxy-6-methyl-2-pyridyl)-4-(trifluoromethyl)-2-pyridyl]-4,4-difluoro-azepane (63 mg, 0.13 mmol) in methanol (1 mL) was stirred with 10% Pd/C (9 mg) under a hydrogen atmosphere for 30 min. Filtration and purification by silica gel chromatography (0-30% methanol/DCM) provided 2-[2-(4,4-difluoroazepan-1-yl)-4-(trifluoromethyl)-3-pyridyl]-6-methyl-1H-pyridin-4-one (318, 19 mg, 73%) as a white solid. (400 MHz, CD₃OD) δ 8.50 (dd, J=5.1, 0.9 Hz, 1H), 7.20 (d, J=5.1 Hz, 1H), 6.42 (d, J=2.4 Hz, 1H), 6.36 (d, J=2.4 Hz, 1H), 3.52 (t, J=5.5 Hz, 2H), 3.36-3.31 (m, 2H), 2.36 (s, 3H), 2.25-1.94 (m, 4H), 1.75 (p, J=6.2 Hz, 2H). ESI-MS m/z calc. 387.14, found 388.0 (M+1)⁺.

### Compound 319

### 2-[2-(4,4-difluoro-1-piperidyl)-4-(trifluoromethyl)-3-pyridyl]-6-methyl-1H-pyridin-4-one

2-[2-(4,4-Difluoro-1-piperidyl)-4-(trifluoromethyl)-3-pyridyl]-6-methyl-1H-pyridin-4-one was synthesized in a manner analogous to Compound 318, using 4,4-difluoropiperidine in step 1. ESI-MS m/z calc. 373.12, found 374.3 (M+1)⁺.

### Compound 320

### 2-[2-(4,4-difluoroazepan-1-yl)-5-methyl-3-pyridyl]-6-methyl-1H-pyridin-4-one

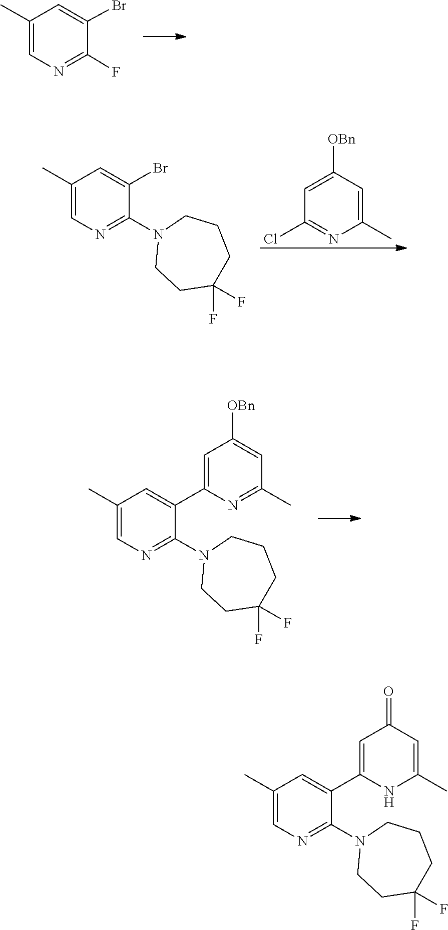

### Step 1: 1-(3-bromo-5-methyl-2-pyridyl)-4,4-difluoro-azepane

A mixture of 3-bromo-2-fluoro-5-methyl-pyridine (650 mg, 3.421 mmol), 4,4-difluoroazepane (555 mg, 4.106 mmol) and DIPEA (1.190 mL, 6.832 mmol) in 1,4-dioxane (1.0 mL) was heated at 80° C. for 60 h in a sealed vial. The mixture was partitioned between ethyl acetate and saturated aqueous ammonium chloride, and the organic phase extracted with additional ethyl acetate (3×). The combined extracts were washed with brine, dried over sodium sulfate, filtered, and concentrated. Purification by silica gel chromatography (0-15% ethyl acetate/hexanes) provided 1-(3-bromo-5-methyl-2-pyridyl)-4,4-difluoro-azepane (38 mg, 4%). ESI-MS m/z calc. 304.04, found 305.0 (M+1)⁺.

### Step 2: 1-[3-(4-benzyloxy-6-methyl-2-pyridyl)-5-methyl-2-pyridyl]-4,4-difluoro-azepane

1-[3-(4-Benzyloxy-6-methyl-2-pyridyl)-5-methyl-2-pyridyl]-4,4-difluoro-azepane was prepared from 1-(3-bromo-5-methyl-2-pyridyl)-4,4-difluoro-azepane and 4-benzyloxy-2-chloro-6-methyl-pyridine using a cross-coupling procedure analogous to that found in Compound 318 step 2. ESI-MS m/z calc. 423.21, found 424.0 (M+1)⁺.

### Step 3: 2-[2-(4,4-difluoroazepan-1-yl)-5-methyl-3-pyridyl]-6-methyl-1H-pyridin-4-one, Compound 320

2-[2-(4,4-Difluoroazepan-1-yl)-5-methyl-3-pyridyl]-6-methyl-1H-pyridin-4-one was prepared using a debenzylation procedure analogous to that found in Compound 209, step 3. ¹H NMR (500 MHz, CD₃OD) δ 8.11 (d, J=2.4 Hz, 1H), 7.48 (d, J=2.4 Hz, 1H), 6.38 (d, J=2.4 Hz, 1H), 6.29 (d, J=2.4 Hz, 1H), 3.50-3.41 (m, 2H), 3.27 (t, J=6.1 Hz, 2H), 2.36 (s, 3H), 2.28 (s, 3H), 2.17 (m, 2H), 2.07-1.94 (m, 2H), 1.77 (m, 2H). ESI-MS m/z calc. 333.17, found 334.0 (M+1)⁺.

### Compound 321 (Cis/Trans Mixture)

### 2-methyl-6-[2-(trifluoromethyl)-5-[4-(trifluoromethyl)cyclohexyl]-4-pyridyl]-1H-pyridin-4-one and Compound 322 (cis)

### 2-methyl-6-[2-(trifluoromethyl)-5-[4-(trifluoromethyl)cyclohexyl]-4-pyridyl]-1H-pyridin-4-one

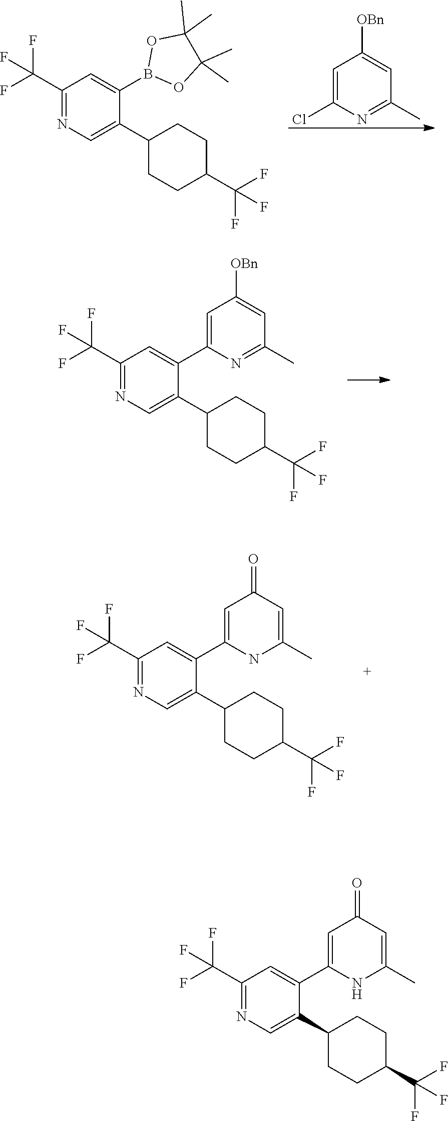

### Step 1: 4-benzyloxy-2-methyl-6-[2-(trifluoromethyl)-5-[4-(trifluoromethyl)cyclohexyl]-4-pyridyl]pyridine

4-Benzyloxy-2-methyl-6-[2-(trifluoromethyl)-5-[4-(trifluoromethyl)cyclohexyl]-4-pyridyl]pyridine (cis/trans mixture) was prepared from 4-benzyloxy-2-chloro-6-methyl-pyridine and 4-(4,4,5,5-tetramethyl-1,3,2-dioxaborolan-2-yl)-2-(trifluoromethyl)-5-[4-(trifluoromethyl)cyclohexyl]pyridine (cis/trans mixture) using a cross-coupling procedure analogous to that found in Compound 209, step 1. ESI-MS m/z calc. 494.18, found 495.5 (M+1)⁺

### Step 2

A solution of 4-benzyloxy-2-methyl-6-[2-(trifluoromethyl)-5-[4-(trifluoromethyl)cyclohexyl]-4-pyridyl]pyridine (cis/trans mixture) from step 1 in methanol (3 mL) was stirred with 10% Pd/C (8.3 mg, 0.0039 mmol). After reaction completion the mixture was filtered and purified by reverse phase HPLC (10-99% acetonitrile/5 mM HCl over 15 min) yielding a 3:1 mixture of cis/trans isomers 2-methyl-6-[2-(trifluoromethyl)-5-[4-(trifluoromethyl)cyclohexyl]-4-pyridyl]-1H-pyridin-4-one (Compound 321, 6.1 mg, 32%). ¹H NMR (400 MHz, CD₃OD) δ 8.97 (s, 0.25H), 8.87 (s, 0.75H), 7.94 (s, 1H), 7.12-7.04 (m, 2H), 2.84-2.70 (m, 0.75H), 2.65 (s, 3H), 2.63-2.56 (m, 0.25H), 2.51-2.37 (m, 0.75H), 2.36-2.24 (m, 0.25H), 2.13-2.01 (m, 2H), 2.01-1.80 (m, 2H), 1.79-1.64 (m, 3.5H), 1.44-1.26 (m, 0.5H). ¹⁹F NMR (376 MHz, CD₃OD) δ −68.21 (major), −69.29 (major) overlaps with −69.29 (minor), −75.39 (minor). ESI-MS m/z calc. 404.13, found 405.3 (M+1)⁺, and the major cis isomer of 2-methyl-6-[2-(trifluoromethyl)-5-[4-(trifluoromethyl)cyclohexyl]-4-pyridyl]-1H-pyridin-4-one (Compound 322, 2.2 mg, 12%). ¹H NMR (400 MHz, CD₃OD) δ 8.88 (s, 1H), 7.94 (s, 1H), 7.10 (d, J=2.4 Hz, 1H), 7.08 (d, J=2.4 Hz, 1H), 2.80-2.69 (m, 1H), 2.66 (s, 3H), 2.52-2.36 (m, 1H), 2.09 (d, J=14.5 Hz, 2H), 1.89 (q, J=12.8, 12.1 Hz, 2H), 1.79-1.64 (m, 4H). ¹⁹F NMR (376 MHz, CD₃OD) δ −68.21, −69.29. ESI-MS m/z calc. 404.13, found 405.2 (M+1)⁺.

### Compound 323

### 2-[2-(4,4-difluoro-1-hydroxy-cyclohexyl)-4-(trifluoromethyl)phenyl]-6-methyl-1H-pyridin-4-one

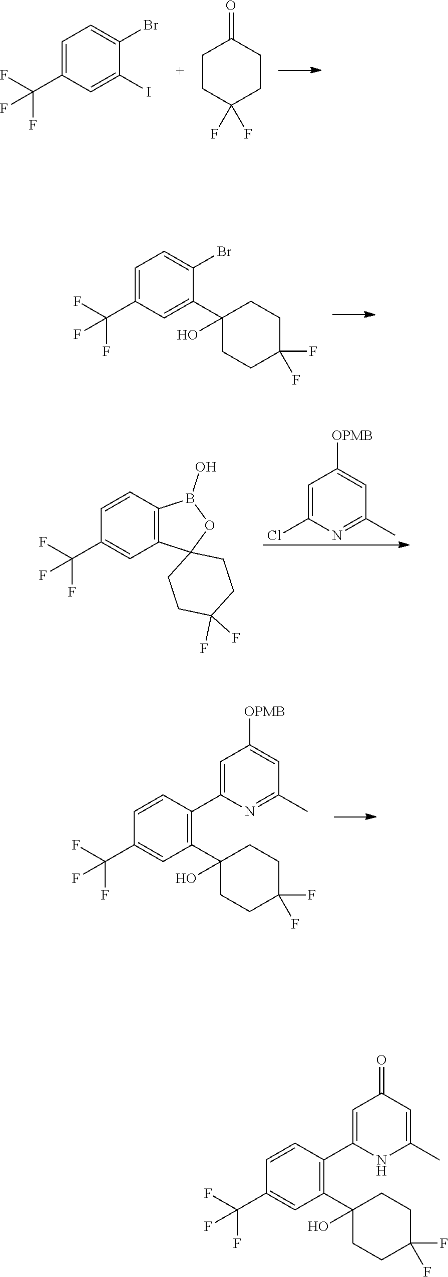

### Step 1: 1-[2-bromo-5-(trifluoromethyl)phenyl]-4,4-difluoro-cyclohexanol

To a solution of 1-bromo-2-iodo-4-(trifluoromethyl)benzene (1.036 g, 2.952 mmol) in THF (8 mL) at −10° C. was slowly added isopropylmagnesium chloride lithium chloride complex solution (2.3 mL of 1.3 M, 3.0 mmol) and the reaction mixture stirred at that temperature for 30 min. A solution of 4,4-difluorocyclohexanone (436 mg, 3.25 mmol) in THF (4 mL) was added dropwise to the reaction mixture, which was then removed from the ice bath and stirred for 1 hour at room temperature. The mixture was diluted with saturated aqueous ammonium chloride and partitioned between ethyl acetate and water. The organic phase was separated, dried over sodium sulfate, filtered and concentrated. Purification by silica gel chromatography (80 g silica, 0-30% ethyl acetate/hexane) provided 1-[2-bromo-5-(trifluoromethyl)phenyl]-4,4-difluoro-cyclohexanol (620 mg, 58%). ¹H NMR (400 MHz, CDCl₃) δ 7.90 (d, J=1.9 Hz, 1H), 7.74 (d, J=8.3 Hz, 1H), 7.40 (dd, J=8.1, 1.7 Hz, 1H), 2.63-2.52 (m, 3H), 2.43-2.22 (m, 2H), 2.15-2.05 (m, 4H).

### Step 2: 4′,4′-difluoro-1-hydroxy-5-(trifluoromethyl)spiro[2,1-benzoxaborole-3,1′-cyclohexane]

A solution of n-BuLi (1.47 mL of 2.5 M in hexane, 3.7 mmol) in diethyl ether (4 mL) was slowly added to a solution of 1-[2-bromo-5-(trifluoromethyl)phenyl]-4,4-difluoro-cyclohexanol (599 mg, 1.67 mmol) in diethyl ether (8 mL) at −78° C. The reaction mixture was stirred for 1.5 h, then a solution of triisopropyl borate (520 μL, 2.267 mmol) in diethyl ether (5 mL) slowly added at −78° C. over 10 min. The reaction mixture was removed from the dry ice bath and allowed to come to room temperature. After 2 h, the reaction mixture was diluted with aqueous HCl (17 mL of 1 M, 17 mmol) and stirred for 1 hour. The organic layer was separated and extracted with aqueous NaOH (17 mL of 1 M, 17 mmol). The basic aqueous layer was acidified with aqueous HCl (20 mL of 1 M, 20 mmol) and extracted with diethyl ether (3×). The combined organic layers were dried over sodium sulfate, filtered and concentrated to provide 4′,4′-difluoro-1-hydroxy-5-(trifluoromethyl)spiro[2,1-benzoxaborole-3,1-cyclohexane](440 mg, 86%).

### Step 3: 2-[2-(4,4-difluoro-1-hydroxy-cyclohexyl)-4-(trifluoromethyl)phenyl]-6-methyl-1H-pyridin-4-one, Compound 323

4,4-Difluoro-1-[2-[4-[(4-methoxyphenyl)methoxy]-6-methyl-2-pyridyl]-5-(trifluoromethyl)phenyl]cyclohexanol was prepared from 2-chloro-4-[(4-methoxyphenyl)methoxy]-6-methyl-pyridine and 4′,4′-difluoro-1-hydroxy-5-(trifluoromethyl)spiro[2,1-benzoxaborole-3,1′-cyclohexane] using a cross-coupling procedure analogous to that found in Compound 209, step 1 using Pd(PPh₃)₄ as the catalyst. ESI-MS m/z calc. 507.18, found 508.3 (M+1)⁺. The PMB-protected intermediate was dissolved in methanol and stirred with 10% Pd/C (wet, 12 mg, 0.0056 mmol) under hydrogen atmosphere for 20 min. Filtration and purification provided 2-[2-(4,4-difluoro-1-hydroxy-cyclohexyl)-4-(trifluoromethyl)phenyl]-6-methyl-1H-pyridin-4-one. ESI-MS m/z calc. 387.13, found 388.2 (M+1)⁺. ¹H NMR (400 MHz, CD₃OD) δ 7.83 (s, 1H), 7.76 (dd, J=8.3, 1.8 Hz, 1H), 7.53 (d, J=8.0 Hz, 1H), 7.04 (dd, J=2.4, 0.7 Hz, 1H), 6.97 (d, J=2.4 Hz, 1H), 2.62 (s, 3H), 2.24-2.04 (m, 4H), 2.02-1.88 (m, 4H).

### Compound 324

### 2-[2-(4,4-difluorocyclohexen-1-yl)-4-(trifluoromethyl)phenyl]-6-methyl-1H-pyridin-4-one and

### Compound 325

### 2-[2-(4,4-difluorocyclohexyl)-4-(trifluoromethyl)phenyl]-6-methyl-1H-pyridin-4-one

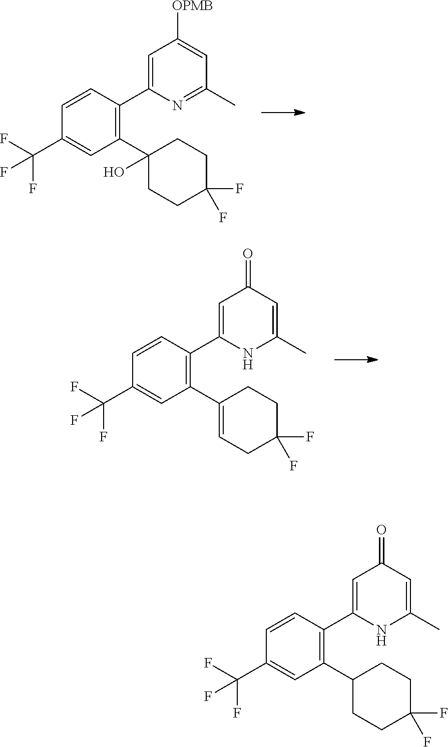

A mixture of 4,4-difluoro-1-[2-[4-[(4-methoxyphenyl)methoxy]-6-methyl-2-pyridyl]-5-(trifluoromethyl)phenyl]cyclohexanol (50 mg, 0.099 mmol) and 4-methylbenzenesulfonic acid hydrate (56 mg, 0.29 mmol) were combined in toluene (4 mL) and refluxed for 1 h. The mixture was concentrated in vacuo and purified by reverse phase HPLC (C18, 10-99% acetonitrile/5 mM HCl). The combined product fractions were diluted with ethyl acetate and washed with 50% saturated sodium bicarbonate (2×). The organic phase was dried over sodium sulfate, filtered and concentrated in vacuo to provide 2-[2-(4,4-difluorocyclohexen-1-yl)-4-(trifluoromethyl)phenyl]-6-methyl-1H-pyridin-4-one (Compound 324, 30 mg, 82%). ESI-MS m/z calc. 369.12, found 370.2 (M+1)⁺. The alkene product 2-[2-(4,4-difluorocyclohexen-1-yl)-4-(trifluoromethyl)phenyl]-6-methyl-1H-pyridin-4-one (12 mg, 0.03249 mmol) was stirred with 10% Pd/C (wet, 8 mg, 0.004 mmol) in methanol (3 mL) under hydrogen atmosphere for 2 h. The mixture was filtered and concentrated to provide 2-[2-(4,4-difluorocyclohexyl)-4-(trifluoromethyl)phenyl]-6-methyl-1H-pyridin-4-one (Compound 325, 11 mg, 91%). ESI-MS m/z calc. 371.13, found 372.2 (M+1)⁻.

### Example 12

### 6-(2-(3,4-difluoro-2-methylphenoxy)-4-methyl-5-(trifluoromethyl)pyridin-3-yl)-1-(methylimino)-1,3,4,5-tetrahydro-1λ4-thiopyrano[3,2-b]pyridin-8(2H)-one 1-oxide (326)

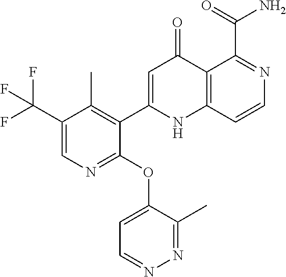 [SMILES: Cc1nnccc1Oc1ncc(C(F)(F)F)c(C)c1-c1cc(=O)c2c(C(N)=O)nccc2[nH]1]

6-(2-(3,4-difluoro-2-methylphenoxy)-4-methyl-5-(trifluoromethyl)pyridin-3-yl)-1-(methylimino)-1,3,4,5-tetrahydro-1λ4-thiopyrano[3,2-b]pyridin-8(2H)-one 1-oxide (326) can be prepared by the methods described herein.

### Example 13

6-(2-(3,4-difluoro-2-methylphenoxy)-4-methyl-5-(trifluoromethyl)pyridin-3-yl)-1-(methylimino)-1,3,4,5-tetrahydro-1λ4-thiopyrano[3,2-b]pyridin-8(2H)-one 1-oxide (327)

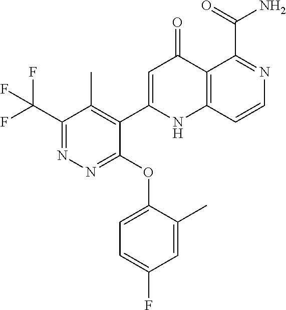 [SMILES: Cc1cc(F)ccc1Oc1nnc(C(F)(F)F)c(C)c1-c1cc(=O)c2c(C(N)=O)nccc2[nH]1]

6-(2-(3,4-difluoro-2-methylphenoxy)-4-methyl-5-(trifluoromethyl)pyridin-3-yl)-1-(methylimino)-1,3,4,5-tetrahydro-1,4-thiopyrano[3,2-b]pyridin-8(2H)-one 1-oxide (327) can be prepared by the methods described herein.

### Example 14

6-(2-(3,4-difluoro-2-methylphenoxy)-4-methyl-5-(trifluoromethyl)pyridin-3-yl)-3,4-dihydro-2H-thiopyrano[3,2-b]pyridin-8(5H)-one 1,1-dioxide (328)

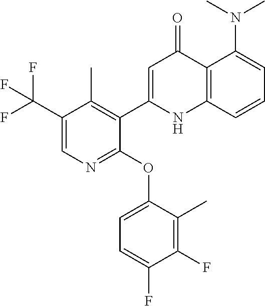 [SMILES: Cc1c(Oc2ncc(C(F)(F)F)c(C)c2-c2cc(=O)c3c(N(C)C)cccc3[nH]2)ccc(F)c1F]

6-(2-(3,4-difluoro-2-methylphenoxy)-4-methyl-5-(trifluoromethyl)pyridin-3-yl)-3,4-dihydro-2H-thiopyrano[3,2-b]pyridin-8(5H)-one 1,1-dioxide (328) can be prepared by the methods described herein.

### Example 15

### E-VIPR Assay Detecting and Measuring Na_{V} Inhibition Properties

Sodium ion channels are voltage-dependent proteins that can be activated by inducing membrane voltage changes by applying electric fields. The electrical stimulation instrument and methods of use, referred to as E-VIPR, are described in International Publication No. WO 2002/008748 A3 and C.-J. Huang et al. Characterization of voltage-gated sodium channel blockers by electrical stimulation and fluorescence detection of membrane potential, 24 Nature Biotech. 439-46 (2006), both of which are incorporated by reference in their entirety. The instrument comprises a microtiter plate handler, an optical system for exciting the coumarin dye while simultaneously recording the coumarin and oxonol emissions, a waveform generator, a current- or voltage-controlled amplifier, and parallel electrode pairs that are inserted into assay plate wells. Under integrated computer control, this instrument passes user-programmed electrical stimulus protocols to cells within the wells of the microtiter plate.

16-20 hours prior to running the assay on E-VIPR, HEK cells expressing a truncated form of human Na_{V} 1.8 with full channel activity were seeded into microtiter 384-well plates, pre-coated with matrigel, at a density of 25,000 cells per well. 2.5-5% KIR2.1 BacMam virus was added to the final cell suspension before seeding into cell plates. HEK cells were grown in Dulbecco's Modified Eagle's Medium (DMEM) supplemented with 10% FBS (Fetal Bovine Serum, qualified; Sigma #F4135), 1% NEAA (Non-Essential Amino Acids, Gibco #11140), 1% HEPES (Gibco #15630), 1% Pen-Strep (Penicillin-Streptomycin; Gibco #15140) and 5 μg/ml Blasticidin (Gibco #R210-01). Cells were expanded in 5-layer CellSTACK culture chambers or cell culture flasks with vented caps, with 90-95% humidity and 5% CO₂.

### Reagents and Stock Solutions:

100 mg/mL Pluronic F-127 (Sigma #P2443), in dry DMSO

Compound Plates: Corning 384-well Polypropylene Round Bottom #3656

Cell Plates: 384-well tissue culture treated plates (Greiner #781091-2B)

2.5-5% KIR 2.1 Bacmam virus (produced in-house), prepared as described in Section 3.3 of J. A. Fornwald et al., Gene Expression in Mammalian Cells Using BacMam, a Modified Baculovirus System, 1350 Methods in Molecular Biology 95-116 (2016), the entire contents of which are incorporated by reference. The concentration used can be dependent on viral titer of each batch.

5 mM DiSBAC₆(3), a voltage sensitive oxonol acceptor (CAS number 169211-44-3; 5-[3-(1,3-dihexylhexahydro-4,6-dioxo-2-thioxo-5-pyrimidinyl)-2-propen-1-ylidene]-1,3-dihexyldihydro-2-thioxo-4,6(1H,5H)-pyrimidinedione), in dry DMSO. The preparation of DiSBAC₆(3) is analogous to that of DiSBAC₄(3) as described in Voltage Sensing by Fluorescence Resonance Energy Transfer in Single Cells, Gonzalez, J. E. and Tsien, R. Y. (1995) Biophys. J. 69, 1272-1280.

5 mM CC2-DMPE, a commercially available membrane-bound coumarin phospholipid FRET donor (ThermoFisher Scientific catalog number K1017, CAS number 393782-57-5; tetradecanoic acid, 1,1′-[(1R)-1-[8-(6-chloro-7-hydroxy-2-oxo-2H-1-benzopyran-3-yl)-3-hydroxy-3-oxido-8-oxo-2,4-dioxa-7-aza-3-phosphaoct-1-yl]-1,2-ethanediyl]ester) was prepared in dry DMSO. See also, Improved indicators of cell membrane potential that use fluorescence resonance energy transfer, Gonzalez, J. E. and Tsien, R. Y. (1997) Chem. Biol. 4, 269-277.

Voltage Assay Background Suppression Compound (VABSC-1) is prepared in H₂O (89-363 mM, range used to maintain solubility)

Human Serum (HS, Millipore #S1P1-01KL, or Sigma SLBR5469V and SLBR5470V as a 50%/50% mixture, for 25% assay final concentration)

### Bath 1 Buffer:

Sodium Chloride 160 mM (9.35 g/L), Potassium Chloride, 4.5 mM (0.335 g/L), Glucose 10 mM (1.8 g/L), Magnesium Chloride (Anhydrous) 1 mM (0.095 g/L), Calcium Chloride 2 mM (0.222 g/L), HEPES 10 mM (2.38 g/L) in water.

### Na/TMA Cl Bath 1 Buffer:

Sodium Chloride 96 mM (5.61 g/L), Potassium Chloride 4.5 mM (0.335 g/L), Tetramethylammonium (TMA)-Cl 64 mM (7.01 g/L), Glucose 10 mM (1.8 g/L), Magnesium Chloride (Anhydrous) 1 mM (0.095 g/L), Calcium Chloride 2 mM (0.222 g/L) HEPES 10 mM (2.38 g/L) in water.

### Hexyl Dye Solution (2× Concentration):

Bath 1 Buffer containing 0.5% β-cyclodextrin (made fresh prior to each use, Sigma #C4767), 8 M CC2-DMPE and 2 μM DiSBAC₆(3). The solution was made by adding 10% Pluronic F127 stock equal to combined volumes of CC2-DMPE and DiSBAC₆(3). The order of preparation was first mix Pluronic and CC2-DMPE, then add DiSBAC₆(3), then while vortexing add Bath I/O-Cyclodextrin.

Compound Loading Buffer (2× concentration): Na/TMA Cl Bath1 Buffer containing HS (omitted in experiments run in the absence of human serum (HS)) 50%, VABSC-1 1 mM, BSA 0.2 mg/ml (in Bath-1), KCl 9 mM, DMSO 0.625%.

### Assay Protocol (7 Key Steps):

1) To reach the final concentration in each well, 375 nL of each compound was pre-spotted (in neat DMSO) into polypropylene compound plates at 240× desired final concentration from an intermediate stock concentration of 0.075 mM, in an 11-point dose response, 3-fold dilution, resulting in a top dose of 300 nM final concentration in the cell plate. Vehicle control (neat DMSO), and positive control (an established Na_{V}1.8 inhibitor, 25 M final in assay in DMSO) were added manually to the outermost columns of each plate respectively. The compound plate was backfilled with 45 μL per well of Compound Loading Buffer resulting in a 240-fold dilution of compound following a 1:1 transfer of compound into the cell plate (see Step 6). Final DMSO concentration for all wells in the assay was 0.625% (0.75% DMSO was supplemented to the Compound Loading Buffer for a final DMSO concentration of 0.625%). This assay dilution protocol was adjusted to enable a higher dose range to be tested in the presence of HS or if the final assay volume was altered.
2) Hexyl Dye Solution was prepared.
3) Cell plates were prepared. On the day of the assay, the media was aspirated, and the cells were washed three times with 80 μL of Bath-1 buffer, maintaining 25 μL residual volume in each well.
4) 25 μL per well of Hexyl Dye Solution was dispensed into the cell plates. The cells were incubated for 20 minutes at room temperature or ambient conditions in darkness.
5) 45 μL per well of Compound Loading Buffer was dispensed into compound plates.
6) The cell plates were washed three times with 80 μL per well of Bath-1 Buffer, leaving L of residual volume. Then 25 μL per well from compound plate was transferred to each cell plate. The mixture was incubated for 30 minutes at room temperature/ambient conditions.
7) The cell plate containing compound was read on E-VIPR using the current-controlled amplifier to deliver stimulation wave pulses using a symmetrical biphasic waveform. The user-programmed electrical stimulus protocols were 1.25-4 Amps and 4 millisecond pulse width (dependent on electrode composition) were delivered at 10 Hz for 10 seconds. A pre-stimulus recording was performed for each well for 0.5 seconds to obtain the un-stimulated intensities baseline. The stimulatory waveform was followed by 0.5 seconds of post-stimulation recording to examine the relaxation to the resting state. All E-VIPR responses were measured at 200 Hz acquisition rate.

### Data Analysis:

Data were analyzed and reported as normalized ratios of emission intensities measured in the 460 nm and 580 nm channels. The response as a function of time was reported as the ratios obtained using the following formula:

$$R ( t ) = \frac{( {intensity}_{460 n m} )}{( {intensity}_{580 n m} )}$$

The data were normalized by calculating the initial (Rᵢ) and final (R_{f}) ratios. These were the average ratio values during part or all of the pre-stimulation period and during sample points during the stimulation period. The fluorescence ratio (R_{f}/Rᵢ) was then calculated and reported as a function of time.

Control responses were obtained by performing assays in the presence of the positive control, and in the absence of pharmacological agents (DMSO vehicle negative control). Responses to the negative (N) and positive (P) controls were calculated as above. The compound antagonist % activity A was then defined as:

$$A = \frac{X - N}{P - N} \times 100$$

where X is the maximum amplitude of the ratio response or number of action potential peaks, at the beginning of the pulse train in the presence of test compound. Using this analysis protocol, dose response curves were plotted and IC₅₀ values were generated for various compounds of the present invention.

Compounds having a measured IC₅₀ value less than 0.5 μM in the E-VIPR Assay described above include: 1-33, 35-42, 44-54, 56-104, 106-108, 110-114, 116-134, 137-141, 143, 144, 147, 149-155, 158, 159, 161, 163-223, 225, 230, 232-237, 239-241, 243, 245-248, 251, 254, 257-273, 275-279, 281-283, 285-288, 290-296, 298-309, 311-317, and 326-328.

Compounds having a measured IC₅₀ value less than 2 μM and greater than or equal to 0.5 μM in the E-VIPR Assay described above include: 43, 115, 135, 142, 156, 162, 238, 250, 253, 256, 280, 284, 289, 310, and 325.

Compounds having a measured IC₅₀ value less than 5 μM and greater than or equal to 2 μM in the E-VIPR Assay described above include: 34, 109, 136, 224, 226, 227, 229, 231, 242, 244, 297, 320, and 322.

Compounds having a measured IC₅₀ value greater than or equal to 5 μM in the E-VIPR Assay described above include: 55, 105, 146, 148, 157, 160, 228, 249, 252, 255, 274, 318, 319, 321, 323, and 324.

Many modifications and variations of the embodiments described herein may be made without departing from the scope, as is apparent to those skilled in the art. The specific embodiments described herein are offered by way of example only.

1. A compound of formula (I) or (II) 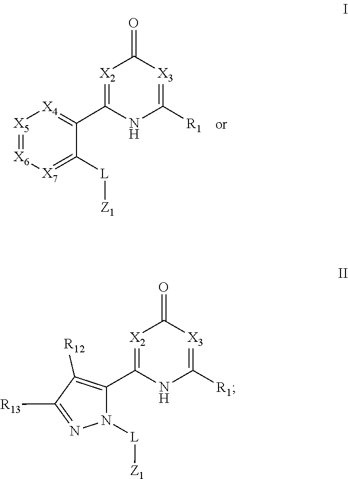
or a pharmaceutically acceptable salt thereof, wherein:
L is O, a single bond, —C(R)₂—, or —C(R)₂—O—;
X₂ is N or CR₂;
X₃ is N or CR₃;
X₄ is N or CR₄;
X₅ is N or CR₅;
X₆ is N or CR₆;
X₇ is N or CR₇;
each R is independently H or C₁-C₆ alkyl;
R₁, R₂, and R₃ are defined as follows:
(i) R₁ is H, halo, CN, OH, C₁-C₆ alkyl, C₁-C₆ alkoxy, (C₁-C₆ alkylene)-OH, NR₈R₉, or CH(OH)(CH₂)ₘ(CHOH)ₙ(CH₂)ₚH; and
R₂ and R₃ are each independently H, halo, CN, OH, C₁-C₆ alkyl optionally substituted with C(O)OR₈, C₂-C₆ alkynyl, C₁-C₆ haloalkyl, C₁-C₆ alkoxy, C₁-C₆ haloalkoxy, (C₁-C₆ alkylene)-OH, NR₈R₉, (C₁-C₆ alkylene)-O—(C₁-C₆ alkyl), C(O)NR₈R₉, CH(OR₈)—C(O)NR₈R₉, C(O)OR₈, CHR₈—C(O)OR₉, CH(OR₈)—C(O)OR₉, CH(OH)(CH₂)ₘ(CHOH)ₙ(CH₂)ₚH, O—(C₁-C₆ alkylene)-O—CH₃, C₁-C₆ alkenyl optionally substituted with C(O)OR₈, S(O)R₈, C(O)C(O)NR₈R₉, CHR₈—C(O)NR₈R₉, C(O)R₁₀, S(═O)(═NR⁸)R⁹, S(O)₂NR₈R₉, 3-7 membered cycloalkyl, 4-7 membered heterocyclyl, or 5-6 membered heteroaryl, wherein said cycloalkyl, heterocyclyl, or heteroaryl in said 3-7 membered cycloalkyl, 4-7 membered heterocyclyl, or 5-6 membered heteroaryl is optionally substituted with 1-4 substituents independently selected from oxo, OH, C₁-C₆ alkyl, C₁-C₆ alkoxy, and C(O)NR₈R₉; or
(ii) R₂ is H; and
R₁ and R₃, together with the carbon atoms to which they are attached, form a ring of formula: 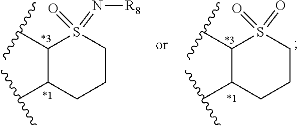
R₄, R₅, R₆, and R₇ are defined as follows:
(i) R₄, R₅, R₆, and R₇ are each independently H, halo, C₁-C₆ alkyl, C₁-C₆ haloalkyl, C₃-C₆ cycloalkyl optionally substituted with halo;
(ii) R₄ and R₇ are each independently is H, halo, C₁-C₆ alkyl, C₁-C₆ haloalkyl, C₃-C₆ cycloalkyl optionally substituted with halo; and R₅ and R₆, together with the carbon atoms to which they are attached, form a ring of formula: 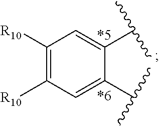
(iii) R₄ and R₇ are each independently is H, halo, C₁-C₆ alkyl, C₁-C₆ haloalkyl, C₃-C₆ cycloalkyl optionally substituted with halo; and R₅ and R₆, together with the carbon atoms to which they are attached, form a ring of formula: 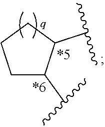
each R₈ and R₉ is independently H or C₁-C₆ alkyl;
each R₁₀ is independently C₁-C₄ alkyl;
each R₁₁ is independently H, halo, C₁-C₄ alkyl, or C₁-C₄ haloalkyl;
R₁₂ and R₁₃ are each independently H, halo, C₁-C₆ alkyl, C₁-C₆ haloalkyl, C₃-C₆ cycloalkyl optionally substituted with halo;
Z₁ is 3-10 membered cycloalkyl, 3-10 membered cycloalkenyl, phenyl, 4-10 membered heterocyclyl, or 5-6 membered heteroaryl, wherein said 3-10 membered cycloalkyl, 3-10 membered cycloalkenyl, phenyl, 4-10 membered heterocyclyl, or 5-6 membered heteroaryl may be unsubstituted or may be substituted with 1-4 substituents selected from halo, OH, CH₂OH, C₁-C₆ alkyl, C₁-C₆ alkoxy, C₁-C₆ haloalkyl, and C₁-C₆ haloalkoxy;
m, n, and p are each independently 0 or 1; and
q is 1, 2, or 3;
wherein when X₃ is CR₃, and R₃ is C(O)OR₈, then:
L is O, or
L is a bond, and X₅ is N, or
L is a bond, and X₇ is N;
wherein when X₂ or X₃ is N, then:
L is O, and Z₁ is phenyl, wherein said phenyl may be unsubstituted or may be substituted with 1-4 substituents selected from halo, OH, CH₂OH, C₁-C₆ alkyl, C₁-C₆ alkoxy, C₁-C₆ haloalkyl, and C₁-C₆ haloalkoxy, or
L is a single bond, and Z₁ is 4-10 membered heterocyclyl, wherein said 4-10 membered heterocyclyl may be unsubstituted or may be substituted with 1-4 substituents selected from halo, OH, CH₂OH, C₁-C₆ alkyl, C₁-C₆ alkoxy, C₁-C₆ haloalkyl, and C₁-C₆ haloalkoxy; and
wherein the compound of formula (I) is not:

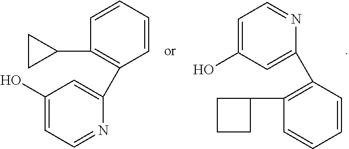 [SMILES: Oc1ccnc(-c2ccccc2C2CC2)c1 ; Oc1ccnc(-c2ccccc2C2CCC2)c1]

2. The compound of formula (I) of claim 1, or a pharmaceutically acceptable salt thereof, wherein:
R₁ is H, halo, CN, OH, C₁-C₆ alkyl, C₁-C₆ alkoxy, (C₁-C₆ alkylene)-OH, NR₈R₉, or CH(OH)(CH₂)ₘ(CHOH)ₙ(CH₂)ₚH; and
R₂ and R₃ are each independently H, halo, CN, OH, C₁-C₆ alkyl optionally substituted with C(O)OR₈, C₂-C₆ alkynyl, C₁-C₆ haloalkyl, C₁-C₆ alkoxy, C₁-C₆ haloalkoxy, (C₁-C₆ alkylene)-OH, NR₈R₉, (C₁-C₆ alkylene)-O—(C₁-C₆ alkyl), C(O)NR₈R₉, CH(OR₈)—C(O)NR₈R₉, C(O)OR₈, CHR₈—C(O)OR₉, CH(OR₈)—C(O)OR₉, CH(OH)(CH₂)ₘ(CHOH)ₙ(CH₂)ₚH, O—(C₁-C₆ alkylene)-O—CH₃, C₁-C₆ alkenyl optionally substituted with C(O)OR₈, S(O)R₈, C(O)C(O)NR₈R₉, CHR₈—C(O)NR₈R₉, C(O)R₁₀, S(═O)(═NR⁸)R⁹, S(O)₂NR₈R₉, 3-7 membered cycloalkyl, 4-7 membered heterocyclyl, or 5-6 membered heteroaryl, wherein said cycloalkyl, heterocyclyl, or heteroaryl in said 3-7 membered cycloalkyl, 4-7 membered heterocyclyl, or 5-6 membered heteroaryl is optionally substituted with 1-4 substituents independently selected from oxo, OH, C₁-C₆ alkyl, C₁-C₆ alkoxy, and C(O)NR₈R₉.

3. The compound of formula (I) of claim 1, or a pharmaceutically acceptable salt thereof, wherein:
R₁ is H, halo, CN, OH, C₁-C₆ alkyl, C₁-C₆ alkoxy, (C₁-C₆ alkylene)-OH, NR₈R₉, or CH(OH)(CH₂)ₘ(CHOH)ₙ(CH₂)ₚH; and
R₂ and R₃ are each independently H, halo, CN, OH, C₁-C₆ alkyl optionally substituted with C(O)OR₈, C₂-C₆ alkynyl, C₁-C₆ haloalkyl, C₁-C₆ alkoxy, C₁-C₆ haloalkoxy, (C₁-C₆ alkylene)-OH, NR₈R₉, (C₁-C₆ alkylene)-O—(C₁-C₆ alkyl), C(O)NR₈R₉, CH(OR₈)—C(O)NR₈R₉, C(O)OR₈, CHR₈—C(O)OR₉, CH(OR₈)—C(O)OR₉, CH(OH)(CH₂)ₘ(CHOH)ₙ(CH₂)ₚH, O—(C₁-C₆ alkylene)-O—CH₃, C₁-C₆ alkenyl optionally substituted with C(O)OR₈, S(O)R₈, C(O)C(O)NR₈R₉, CHR₈—C(O)NR₈R₉, C(O)R₁₀, S(═O)(═NR⁸)R⁹, or S(O)₂NR₈R₉.

4. The compound of formula (I) of claim 1, wherein the compound has formula (I-A) 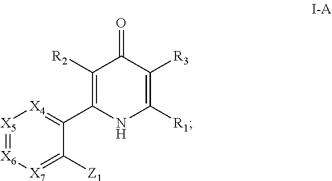
or a pharmaceutically acceptable salt thereof, wherein:
R₁ is H, halo, CN, OH, C₁-C₆ alkyl, C₁-C₆ alkoxy, (C₁-C₆ alkylene)-OH, or NR₈R₉;
R₄, R₅, R₆, and R₇ are each independently H, halo, C₁-C₆ alkyl, C₁-C₆ haloalkyl, C₃-C₆ cycloalkyl optionally substituted with halo; and
Z₁ is 5-10 membered cycloalkyl, phenyl, 4-10 membered heterocyclyl, or 5-6 membered heteroaryl, wherein said 5-10 membered cycloalkyl, phenyl, 4-10 membered heterocyclyl, or 5-6 membered heteroaryl may be unsubstituted or may be substituted with 1-4 substituents selected from CH₂OH, halo, C₁-C₆ alkyl, C₁-C₆ alkoxy, C₁-C₆ haloalkyl, and C₁-C₆ haloalkoxy.

5. The compound of formula (I) of claim 1, or a pharmaceutically acceptable salt thereof, wherein:
at least one of X₄, X₅, X₆, or X₇ is N; and/or
X₄ is CR₄ and X₇ is CR₇; and/or
X₅ is N; and/or
R₃ is C₁-C₆ alkyl; and/or
R₂ and R₃ are each independently H, halo, CN, OH, C₁-C₆ alkyl optionally substituted with C(O)OR₈, C₁-C₆ alkoxy, C₁-C₆ haloalkoxy, C(O)OR₈, S(═O)(═NH)CH₃, S(O)R₈, C(O)R₁₀, or S(O)₂NR₈R₉; and/or
R₄, R₅, R₆, and R₇ are each independently H, halo, C₁-C₆ haloalkyl, or C₃-C₆ cycloalkyl substituted with 1-4 halo; and/or
Z₁ is 5-10 membered cycloalkyl optionally substituted with 1-4 substituents selected from CH₂OH, halo, C₁-C₆ alkyl, C₁-C₆ alkoxy, C₁-C₆ haloalkyl, and C₁-C₆ haloalkoxy; and/or
R₈ is C₃-C₆ alkyl.

6-10. (canceled)

11. The compound of formula (I) of claim 1, or a pharmaceutically acceptable salt thereof, wherein:
R₁ is C₁-C₆ alkyl;
R₂ and R₃ are each independently C₁-C₆ alkyl or C(O)OR₈;
X₅ is N;
R₄, R₅, R₆, and R₇ are each independently H or C₃-C₆ cycloalkyl substituted with 1-2 halo; and
Z₁ is 5-10 membered cycloalkyl substituted with C₁-C₆ haloalkyl.

12-14. (canceled)

15. The compound of formula (I) of claim 1, or a pharmaceutically acceptable salt thereof, wherein:
L is O;
X₂ is CR₂;
X₃ is CR₃; and
R₁ is C₁-C₆ alkyl.

16. The compound of formula (I) of claim 1, or a pharmaceutically acceptable salt thereof, wherein:
L is O; and
Z₁ is phenyl, wherein said phenyl may be unsubstituted or may be substituted with 1-4 substituents selected from CH₂OH, halo, C₁-C₆ alkyl, C₁-C₆ alkoxy, C₁-C₆ haloalkyl, and C₁-C₆ haloalkoxy.

17. (canceled)

18. The compound of formula (I) of claim 1, or a pharmaceutically acceptable salt thereof, wherein:
X₂ is CR₂;
X₃ is CR₃; and
R₂ and R₃ are each independently H, halo, CN, OH, C₁-C₆ alkyl optionally substituted with C(O)OR₈, C₁-C₆ alkoxy, C₁-C₆ haloalkoxy, C(O)OR₈, S(═O)(═NH)CH₃, S(O)R₈, C(O)R₁₀, or S(O)₂NR₈R₉.

19-20. (canceled)

21. The compound of formula (I) of claim 1, or a pharmaceutically acceptable salt thereof, wherein:
X₂ is CR₂;
X₃ is CR₃;
X₄ is CR₄;
X₅ is CR₅;
X₆ is CR₆; and
X₇ is CR₇.

22-27. (canceled)

28. The compound of formula (I) of claim 1, or a pharmaceutically acceptable salt thereof, wherein:
L is a single bond;
X₂ is CR₂;
X₃ is CR₃; and
Z₁ is 4-10 membered heterocyclyl or 5-6 membered heteroaryl, wherein said 4-10 membered heterocyclyl or 5-6 membered heteroaryl may be unsubstituted or may be substituted with 1-4 substituents selected from halo, OH, CH₂OH, C₁-C₆ alkyl, C₁-C₆ alkoxy, C₁-C₆ haloalkyl, and C₁-C₆ haloalkoxy.

29-36. (canceled)

37. The compound of formula (I) of claim 1, or a pharmaceutically acceptable salt thereof, wherein:
R₂ is 3-7 membered cycloalkyl, 4-7 membered heterocyclyl, or 5-6 membered heteroaryl, wherein said cycloalkyl, heterocyclyl, or heteroaryl in said 3-7 membered cycloalkyl, 4-7 membered heterocyclyl, or 5-6 membered heteroaryl is optionally substituted with 1-4 substituents independently selected from oxo, OH, C₁-C₆ alkyl, C₁-C₆ alkoxy, and C(O)NR₈R₉; and
R₃ is H, halo, CN, OH, C₁-C₆ alkyl optionally substituted with C(O)OR₈, C₂-C₆ alkynyl, C₁-C₆ haloalkyl, C₁-C₆ alkoxy, C₁-C₆ haloalkoxy, (C₁-C₆ alkylene)-OH, NR₈R₉, (C₁-C₆ alkylene)-O—(C₁-C₆ alkyl), C(O)NR₈R₉, CH(OR₈)—C(O)NR₈R₉, C(O)OR₈, CHR₈—C(O)OR₉, CH(OR₈)—C(O)OR₉, CH(OH)(CH₂)ₘ(CHOH)ₙ(CH₂)ₚH, O—(C₁-C₆ alkylene)-O—CH₃, C₁-C₆ alkenyl optionally substituted with C(O)OR₈, S(O)R₈, C(O)C(O)NR₈R₉, CHR₈—C(O)NR₈R₉, C(O)R₁₀, S(═O)(═NR⁸)R⁹, or S(O)₂NR₈R₉.

38-43. (canceled)

44. The compound of formula (I) of claim 1, or a pharmaceutically acceptable salt thereof, wherein:
R₂ is H, halo, CN, OH, C₁-C₆ alkyl optionally substituted with C(O)OR₈, C₂-C₆ alkynyl, C₁-C₆ haloalkyl, C₁-C₆ alkoxy, C₁-C₆ haloalkoxy, (C₁-C₆ alkylene)-OH, NR₈R₉, (C₁-C₆ alkylene)-O—(C₁-C₆ alkyl), C(O)NR₈R₉, CH(OR₈)—C(O)NR₈R₉, C(O)OR₈, CHR₈—C(O)OR₉, CH(OR₈)—C(O)OR₉, CH(OH)(CH₂)ₘ(CHOH)ₙ(CH₂)ₚH, O—(C₁-C₆ alkylene)-O—CH₃, C₁-C₆ alkenyl optionally substituted with C(O)OR₈, S(O)R₈, C(O)C(O)NR₈R₉, CHR₈—C(O)NR₈R₉, C(O)R₁₀, S(═O)(═NR⁸)R⁹, or S(O)₂NR₈R₉; and
R₃ is 3-7 membered cycloalkyl, 4-7 membered heterocyclyl, or 5-6 membered heteroaryl, wherein said cycloalkyl, heterocyclyl, or heteroaryl in said 3-7 membered cycloalkyl, 4-7 membered heterocyclyl, or 5-6 membered heteroaryl is optionally substituted with 1-4 substituents independently selected from oxo, OH, C₁-C₆ alkyl, C₁-C₆ alkoxy, and C(O)NR₈R₉.

45-53. (canceled)

54. The compound of formula (II) of claim 1, or a pharmaceutically acceptable salt thereof, wherein:
L is a single bond;
X₂ is CH;
X₃ is CR₃;
R₁ is C₁-C₆ alkyl;
R₃ is 5-6 membered heteroaryl, wherein said heteroaryl is optionally substituted with 1-4 C₁-C₆ alkyl substituents;
R₄ and R₅ are each independently C₁-C₆ alkyl; and
Z₁ is 3-10 membered cycloalkyl, wherein said 3-10 membered cycloalkyl may be unsubstituted or may be substituted with 1-4 C₁-C₆ alkyl substituents.

55. A compound selected from: 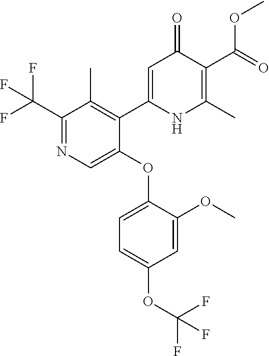 [SMILES: COC(=O)c1c(C)[nH]c(-c2c(Oc3ccc(OC(F)(F)F)cc3OC)cnc(C(F)(F)F)c2C)cc1=O]
methyl 6-5-[2-methoxy-4-
(trifluoromethoxy)phenoxy]-3-methyl-2-
(trifluoromethyl)-4-pyridyl]-2-methyl-4-oxo-1H-
pyridine-3-carboxylate 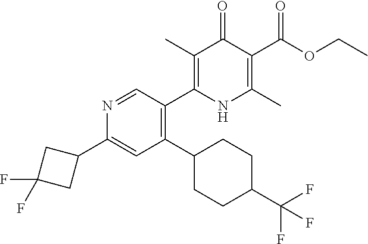 [SMILES: CCOC(=O)c1c(C)[nH]c(-c2cnc(C3CC(F)(F)C3)cc2C2CCC(C(F)(F)F)CC2)c(C)c1=O]
ethyl 6-[6-(3,3-difluorocyclobutyl)-4-[4-
(trifluoromethyl)cyclohexyl]-3-pyridyl]-2,5-
dimethyl-4-oxo-1H-pyridine-3-carboxylate 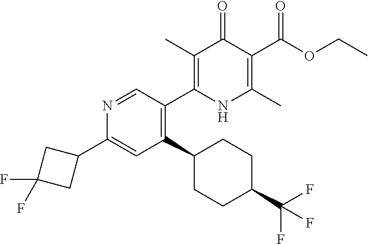 [SMILES: CCOC(=O)c1c(C)[nH]c(-c2cnc(C3CC(F)(F)C3)cc2[C@H]2CC[C@@H](C(F)(F)F)CC2)c(C)c1=O]
ethyl 6-[6-(3,3-difluorocyclobutyl)-4-[(1s,4s)-4-
(trifluoromethyl)cyclohexyl]-3-pyridyl]-2,5-
dimethyl-4-oxo-1H-pyridine-3-carboxylate 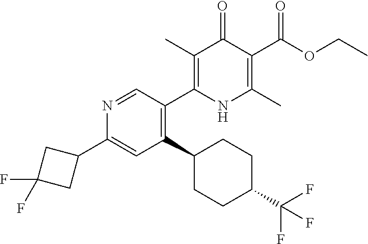 [SMILES: CCOC(=O)c1c(C)[nH]c(-c2cnc(C3CC(F)(F)C3)cc2[C@H]2CC[C@H](C(F)(F)F)CC2)c(C)c1=O]
ethyl 6-[6-(3,3-difluorocyclobutyl)-4-[(1r,4r)-4-
(trifluoromethyl)cyclohexyl]-3-pyridyl]-2,5-
dimethyl-4-oxo-1H-pyridine-3-carboxylate 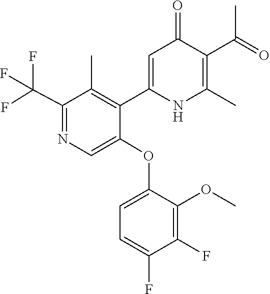 [SMILES: COc1c(Oc2cnc(C(F)(F)F)c(C)c2-c2cc(=O)c(C(C)=O)c(C)[nH]2)ccc(F)c1F]
3-acetyl-6-[5-(3,4-difluoro-2-methoxy-phenoxy)-
3-methyl-2-(trifluoromethyl)-4-pyridyl]-2-methyl-
1H-pyridin-4-one 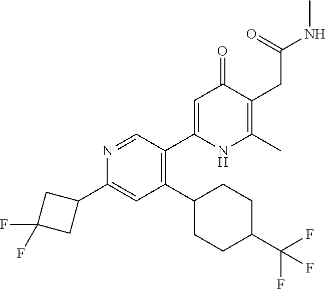 [SMILES: CNC(=O)Cc1c(C)[nH]c(-c2cnc(C3CC(F)(F)C3)cc2C2CCC(C(F)(F)F)CC2)cc1=O]
2-[6-[6-(3,3-difluorocyclobutyl)-4-[4-
(trifluoromethyl)cyclohexyl]-3-pyridyl]-2-methyl-
4-oxo-1H-pyridin-3-yl]-N-methyl-acetamide 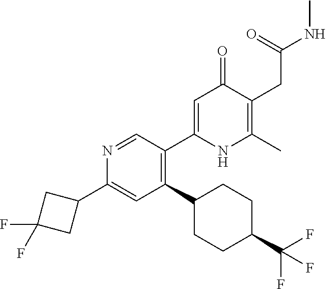
2-[6-[6-(3,3-difluorocyclobutyl)-4-((1s,4s)-4-
(trifluoromethyl)cyclohexyl)-3-pyridyl]-2-methyl-
4-oxo-1H-pyridin-3-yl]-N-methyl-acetamide 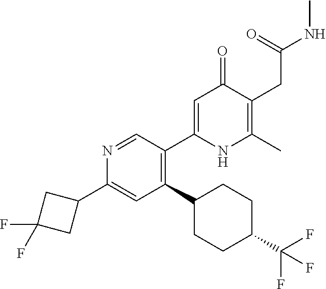
2-[6-[6-(3,3-difluorocyclobutyl)-4-((1r,4r)-4-
(trifluoromethyl)cyclohexyl)-3-pyridyl]-2-methyl-
4-oxo-1H-pyridin-3-yl]-N-methyl-acetamide 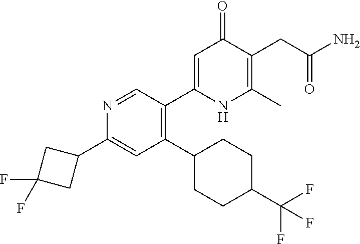 [SMILES: Cc1[nH]c(-c2cnc(C3CC(F)(F)C3)cc2C2CCC(C(F)(F)F)CC2)cc(=O)c1CC(N)=O]
2-[6-[6-(3,3-difluorocyclobutyl)-4-[4-
(trifluoromethyl)cyclohexyl]-3-pyridyl]-2-methyl-
4-oxo-1H-pyridin-3-yl]cetamide 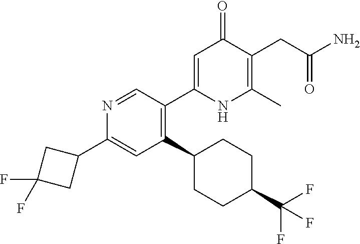 [SMILES: Cc1[nH]c(-c2cnc(C3CC(F)(F)C3)cc2[C@H]2CC[C@@H](C(F)(F)F)CC2)cc(=O)c1CC(N)=O]
2-[6-[6-(3,3-difluorocyclobutyl)-4-((1s,4s)-4-
(trifluoromethyl)cyclohexyl)-3-pyridyl]-2-methyl-
4-oxo-1H-pyridin-3-yl]acetamide 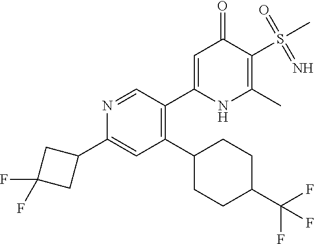 [SMILES: Cc1[nH]c(-c2cnc(C3CC(F)(F)C3)cc2C2CCC(C(F)(F)F)CC2)cc(=O)c1S(C)(=N)=O]
6-[6-(3,3-difluorocyclobutyl)-4-[4-
(trifluoromethyl)cyclohexyl]-3-pyridyl]-2-methyl-
3-(methylsulfonimido)-1H-pyridin-4-one 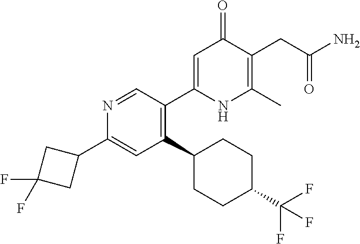 [SMILES: Cc1[nH]c(-c2cnc(C3CC(F)(F)C3)cc2[C@H]2CC[C@H](C(F)(F)F)CC2)cc(=O)c1CC(N)=O]
2-[6-[6-(3,3-difluorocyclobutyl)-4-((1r,4r)-4-
(trifluoromethyl)cyclohexyl]-3-pyridyl]-2-methyl-
4-oxo-1H-pyridin-3-yl]acetamide 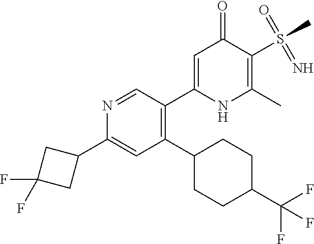 [SMILES: Cc1[nH]c(-c2cnc(C3CC(F)(F)C3)cc2C2CCC(C(F)(F)F)CC2)cc(=O)c1[S@@](C)(=N)=O]
(S)-6-[6-(3,3-difluorocyclobutyl)-4-[4-
(trifluoromethyl)cyclohexyl]-3-pyridyl]-2-methyl-
3-(methylsulfonimido)-1H-pyridin-4-one 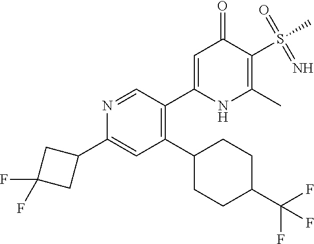 [SMILES: Cc1[nH]c(-c2cnc(C3CC(F)(F)C3)cc2C2CCC(C(F)(F)F)CC2)cc(=O)c1[S@](C)(=N)=O]
(R)-6-[6-(3,3-difluorocyclobutyl)-4-[4-
(trifluoromethyl)cyclohexyl]-3-pyridyl]-2-methyl-
3-(methylsulfonimido)-1H-pyridin-4-one 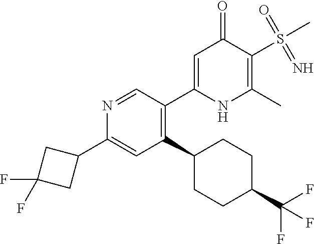 [SMILES: Cc1[nH]c(-c2cnc(C3CC(F)(F)C3)cc2[C@H]2CC[C@@H](C(F)(F)F)CC2)cc(=O)c1S(C)(=N)=O]
6-[6-(3,3-difluorocyclobutyl)-4-((1s,4s)-4-
(trifluoromethyl)cyclohexyl)-3-pyridyl]-2-methyl-
3-(methylsulfonimidoyl)-1H-pyridin-4-one 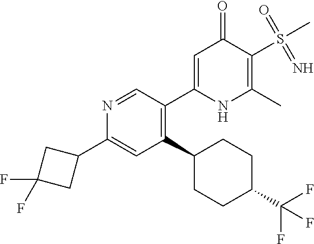 [SMILES: Cc1[nH]c(-c2cnc(C3CC(F)(F)C3)cc2[C@H]2CC[C@H](C(F)(F)F)CC2)cc(=O)c1S(C)(=N)=O]
6-[6-(3,3-difluorocyclobutyl)-4-((1r,4r)-4-
(trifluoromethyl)cyclohexyl)-3-pyridyl]-2-methyl-
3-(methylsulfonimidoyl)-1H-pyridin-4-one 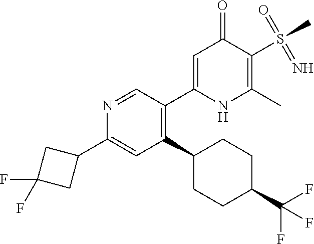 [SMILES: Cc1[nH]c(-c2cnc(C3CC(F)(F)C3)cc2[C@H]2CC[C@@H](C(F)(F)F)CC2)cc(=O)c1[S@@](C)(=N)=O]
6-[6-(3,3-difluorocyclobutyl)-4-((1s,4s)-4-
(trifluoromethyl)cyclohexyl)-3-pyridyl]-2-methyl-
3-((S)-methylsulfonimidoyl)-1H-pyridin-4-one 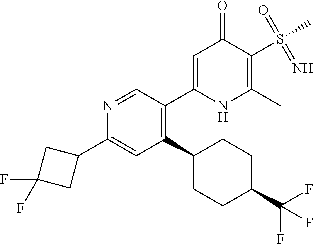 [SMILES: Cc1[nH]c(-c2cnc(C3CC(F)(F)C3)cc2[C@H]2CC[C@@H](C(F)(F)F)CC2)cc(=O)c1[S@](C)(=N)=O]
6-[6-(3,3-difluorocyclobutyl)-4-((1s,4s)-4-
(trifluoromethyl)cyclohexyl)-3-pyridyl]-2-methyl-
3-((R)-methylsulfonimidoyl)-1H-pyridin-4-one 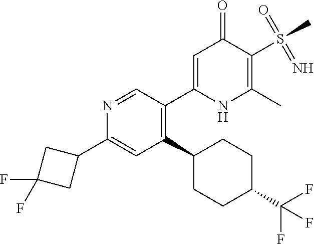 [SMILES: Cc1[nH]c(-c2cnc(C3CC(F)(F)C3)cc2[C@H]2CC[C@H](C(F)(F)F)CC2)cc(=O)c1[S@@](C)(=N)=O]
6-[6-(3,3-difluorocyclobutyl)-4-((1r,4r)-4-
(trifluoromethyl)cyclohexyl)-3-pyridyl]-2-methyl-
3-((S)-methylsulfonimidoyl)-1H-pyridin-4-one 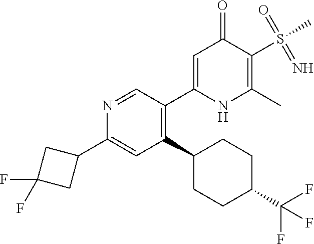 [SMILES: Cc1[nH]c(-c2cnc(C3CC(F)(F)C3)cc2[C@H]2CC[C@H](C(F)(F)F)CC2)cc(=O)c1[S@](C)(=N)=O]
6-[6-(3,3-difluorocyclobutyl)-4-((1r,4r)-4-
(trifluoromethyl)cyclohexyl)-3-pyridyl]-2-methyl-
3-((R)-methylsulfonimidoyl)-1H-pyridin-4-one 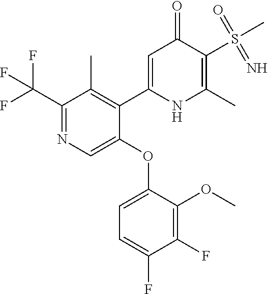 [SMILES: COc1c(Oc2cnc(C(F)(F)F)c(C)c2-c2cc(=O)c(S(C)(=N)=O)c(C)[nH]2)ccc(F)c1F]
6-[5-(3,4-difluoro-2-methoxy-phenoxy)-3-methyl-
2-(trifluoromethyl)-4-pyridyl]-2-methyl-
3-((R)-methylsulfonimidoyl)-1H-pyridin-4-one 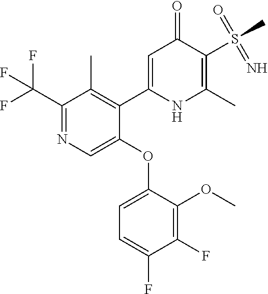 [SMILES: COc1c(Oc2cnc(C(F)(F)F)c(C)c2-c2cc(=O)c([S@@](C)(=N)=O)c(C)[nH]2)ccc(F)c1F]
(S)-6-[5-(3,4-difluoro-2-methoxy-phenoxy)-3-
methly-2-(trifluoromethyl)-4-pyridyl]-2-methyl-3-
(methylsulfonimidoyl)-1H-pyridin-4-one 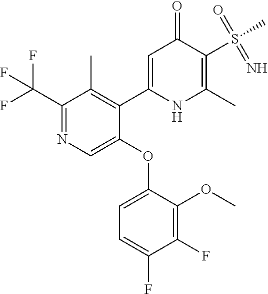 [SMILES: COc1c(Oc2cnc(C(F)(F)F)c(C)c2-c2cc(=O)c([S@](C)(=N)=O)c(C)[nH]2)ccc(F)c1F]
(R)-6-[5-(3,4-difluoro-2-methoxy-phenoxy)-3-
methly-2-(trifluoromethyl)-4-pyridyl]-2-methyl-3-
(methylsulfonimidoyl)-1H-pyridin-4-one 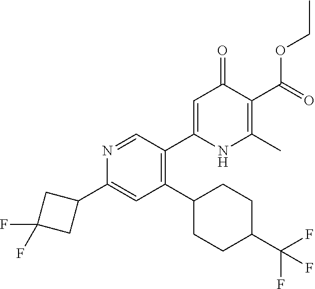 [SMILES: CCOC(=O)c1c(C)[nH]c(-c2cnc(C3CC(F)(F)C3)cc2C2CCC(C(F)(F)F)CC2)cc1=O]
ethyl 6-[6-(3,3-difluorocyclobutyl)-4-[4-
(trifluoromethyl)cyclohexyl]-3-pyridyl]-2-methyl-
4-oxo-1H-pyridine-3-carboxylate 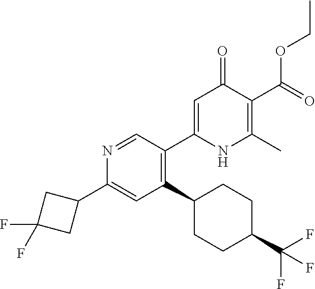 [SMILES: CCOC(=O)c1c(C)[nH]c(-c2cnc(C3CC(F)(F)C3)cc2[C@H]2CC[C@@H](C(F)(F)F)CC2)cc1=O]
ethyl 6-[6-(3,3-difluorocyclobutyl)-4-((1s,4s)-4-
(trifluoromethyl)cyclohexyl)-3-pyridyl]-2-methyl-
4-oxo-1H-pyridine-3-carboxylate 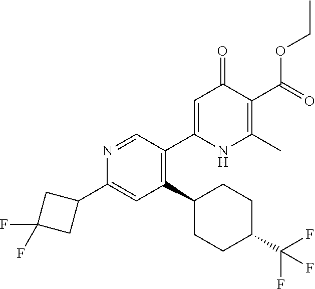 [SMILES: CCOC(=O)c1c(C)[nH]c(-c2cnc(C3CC(F)(F)C3)cc2[C@H]2CC[C@H](C(F)(F)F)CC2)cc1=O]
ethyl 6-[6-(3,3-difluorocyclobutyl)-4-((1r,4r)-4-
(trifluoromethyl)cyclohexyl)-3-pyridyl]-2-methyl-
4-oxo-1H-pyridine-3-carboxylate 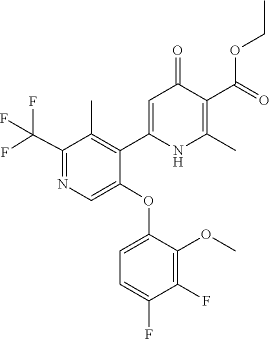 [SMILES: CCOC(=O)c1c(C)[nH]c(-c2c(Oc3ccc(F)c(F)c3OC)cnc(C(F)(F)F)c2C)cc1=O]
ethyl 6-[5-(3,4-difluoro-2-methoxy-phenoxy)-3-
methly-2-(trifluoromethyl)-4-pyridyl]-2-methyl-4-
oxo-1H-pyridine-3-carboxylate 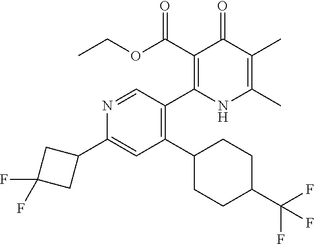 [SMILES: CCOC(=O)c1c(-c2cnc(C3CC(F)(F)C3)cc2C2CCC(C(F)(F)F)CC2)[nH]c(C)c(C)c1=O]
ethyl 2-[6-(3,3-difluorocyclobutyl)-4-(4-
(trifluoromethyl)cyclohexyl)-3-pyridyl]-5,6-
dimethyl-4-oxo-1H-pyridine-3-carboxylate 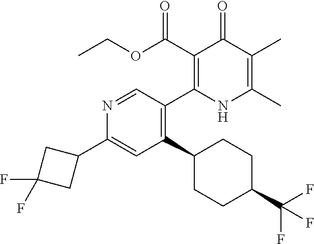 [SMILES: CCOC(=O)c1c(-c2cnc(C3CC(F)(F)C3)cc2[C@H]2CC[C@@H](C(F)(F)F)CC2)[nH]c(C)c(C)c1=O]
ethyl 2-[6-(3,3-difluorocyclobutyl)-4-((1s,4s)-4-
(trifluoromethyl)cyclohexyl)-3-pyridyl]-5,6-
dimethyl-4-oxo-1H-pyridine-3-carboxylate 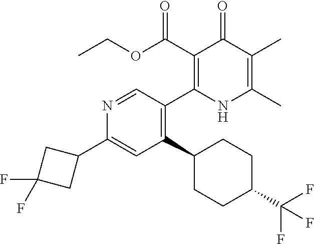 [SMILES: CCOC(=O)c1c(-c2cnc(C3CC(F)(F)C3)cc2[C@H]2CC[C@H](C(F)(F)F)CC2)[nH]c(C)c(C)c1=O]
ethyl 2-[6-(3,3-difluorocyclobutyl)-4-((1r,4r)-4-
(trifluoromethyl)cyclohexyl)-3-pyridyl]-5,6-
dimethyl-4-oxo-1H-pyridine-3-carboxylate 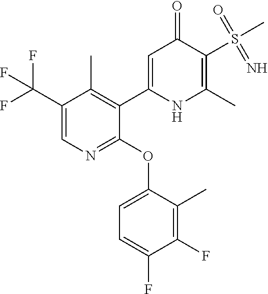 [SMILES: Cc1[nH]c(-c2c(Oc3ccc(F)c(F)c3C)ncc(C(F)(F)F)c2C)cc(=O)c1S(C)(=N)=O]
6-[2-(3,4-difluoro-2-methyl-phenoxy)-4-methyl-
5-(trifluoromethyl)-3-pyridyl]-2-methyl-3-
methylsulfonimidoyl)-1H-pyridin-4-one 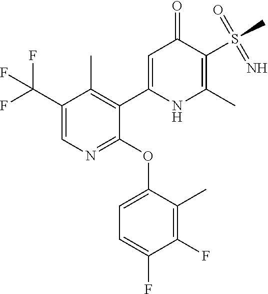 [SMILES: Cc1[nH]c(-c2c(Oc3ccc(F)c(F)c3C)ncc(C(F)(F)F)c2C)cc(=O)c1[S@@](C)(=N)=O]
(S)-6-[2-(3,4-difluoro-2-methyl-phenoxy)-3-
methly-2-(trifluoromethyl)-4-pyridyl]-2-methyl-3-
(methylsulfonimidoyl)-1H-pyridin-4-one 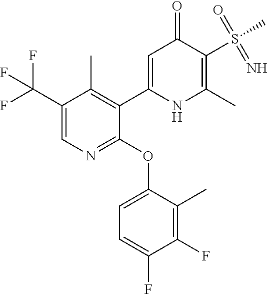 [SMILES: Cc1[nH]c(-c2c(Oc3ccc(F)c(F)c3C)ncc(C(F)(F)F)c2C)cc(=O)c1[S@](C)(=N)=O]
(R)-6-[2-(3,4-difluoro-2-methyl-phenoxy)-4-
methly-5-(trifluoromethyl)-3-pyridyl]-2-methyl-3-
(methylsulfonimidoyl)-1H-pyridin-4-one 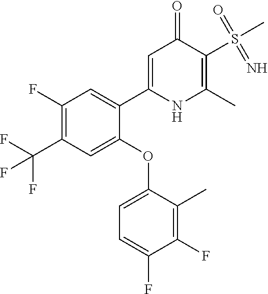 [SMILES: Cc1[nH]c(-c2cc(F)c(C(F)(F)F)cc2Oc2ccc(F)c(F)c2C)cc(=O)c1S(C)(=N)=O]
6-[2-(3,4-difluoro-2-methyl-phenoxy)-5-fluoro-4-
(trifluoromethyl)phenyl]-2-methyl-3-
(methylsulfonimidoyl)-1H-pyridin-4-one 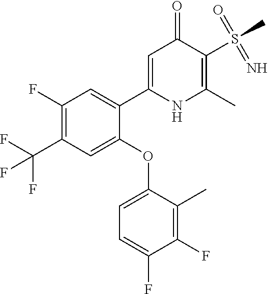 [SMILES: Cc1[nH]c(-c2cc(F)c(C(F)(F)F)cc2Oc2ccc(F)c(F)c2C)cc(=O)c1[S@@](C)(=N)=O]
(S)-6-[2-(3,4-difluoro-2-methyl-phenoxy)-5-
fluoro-4-(trifluoromethyl)phenyl]-2-methyl-3-
(methylsulfonimidoyl)-1H-pyridin-4-one 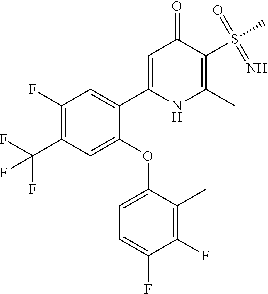 [SMILES: Cc1[nH]c(-c2cc(F)c(C(F)(F)F)cc2Oc2ccc(F)c(F)c2C)cc(=O)c1[S@](C)(=N)=O]
(R)-6-[2-(3,4-difluoro-2-methyl-phenoxy)-5-
fluoro-4-(trifluoromethyl)phenyl]-2-methyl-3-
(methylsulfonimidoyl)-1H-pyridin-4-one 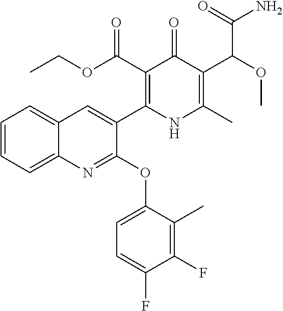 [SMILES: CCOC(=O)c1c(-c2cc3ccccc3nc2Oc2ccc(F)c(F)c2C)[nH]c(C)c(C(OC)C(N)=O)c1=O]
ethyl 5-(2-amino-1-methoxy-2-oxo-ethyl)-2-[2-
(3,4-difluoro-2-methyl-phenoxy)-3-quinolyl]-6-
methyl-4-oxo-1H-pyridine-3-carboxylate 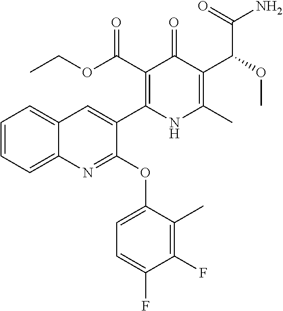 [SMILES: CCOC(=O)c1c(-c2cc3ccccc3nc2Oc2ccc(F)c(F)c2C)[nH]c(C)c([C@@H](OC)C(N)=O)c1=O]
ethyl (R)-5-(2-amino-1-methoxy-2-oxo-ethyl)-2-
[2-(3,4-difluoro-2-methyl-phenoxy)-3-quinolyl]-
6-methyl-4-oxo-1H-pyridine-3-carboxylate 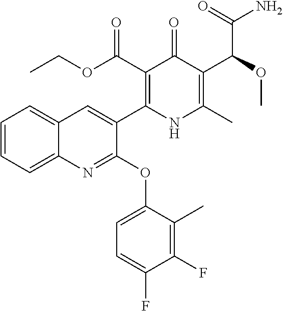 [SMILES: CCOC(=O)c1c(-c2cc3ccccc3nc2Oc2ccc(F)c(F)c2C)[nH]c(C)c([C@H](OC)C(N)=O)c1=O]
ethyl (S)-5-(2-amino-1-methoxy-2-oxo-ethyl)-2-
[2-(3,4-difluoro-2-methyl-phenoxy)-3-quinolyl]-
6-methyl-4-oxo-1H-pyridine-3-carboxylate 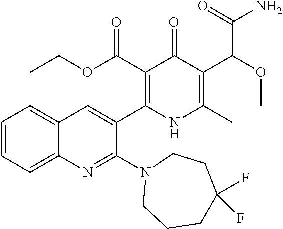 [SMILES: CCOC(=O)c1c(-c2cc3ccccc3nc2N2CCCC(F)(F)CC2)[nH]c(C)c(C(OC)C(N)=O)c1=O]
ethyl 5-(2-amino-1-methoxy-2-oxo-ethyl)-2-[2-
(4,4-difluoroazepan-1-yl)-3-quinolyl]-6-methyl-4-
oxo-1H-pyridine-3-carboxylate 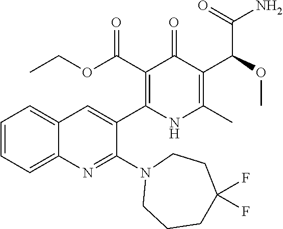 [SMILES: CCOC(=O)c1c(-c2cc3ccccc3nc2N2CCCC(F)(F)CC2)[nH]c(C)c([C@H](OC)C(N)=O)c1=O]
ethyl (S)-5-(2-amino-1-methoxy-2-oxo-ethyl)-2-
[2-(4,4-difluoroazepan-1-yl)-3-quinolyl]-6-
methyl-4-oxo-1H-pyridine-3-carboxylate 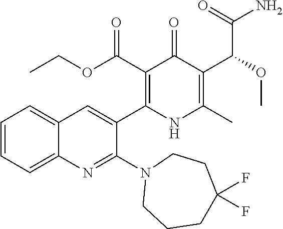 [SMILES: CCOC(=O)c1c(-c2cc3ccccc3nc2N2CCCC(F)(F)CC2)[nH]c(C)c([C@@H](OC)C(N)=O)c1=O]
ethyl (R)-5-(2-amino-1-methoxy-2-oxo-ethyl)-2-
[2-(4,4-difluoroazepan-1-yl)-3-quinolyl]-6-
methyl-4-oxo-1H-pyridine-3-carboxylate 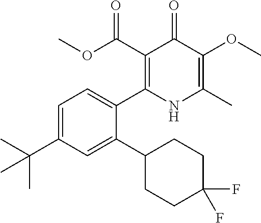 [SMILES: COC(=O)c1c(-c2ccc(C(C)(C)C)cc2C2CCC(F)(F)CC2)[nH]c(C)c(OC)c1=O]
methyl 2-[4-tert-butyl-2-(4,4-
difluorocyclohexyl)phenyl]-5-methoxy-6-methyl
4-oxo-1H-pyridine-3-carboxylate 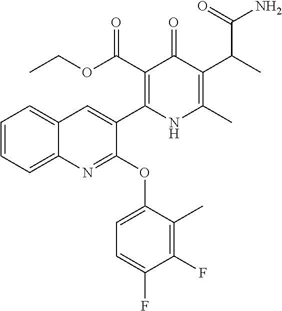 [SMILES: CCOC(=O)c1c(-c2cc3ccccc3nc2Oc2ccc(F)c(F)c2C)[nH]c(C)c(C(C)C(N)=O)c1=O]
ethyl 5-(2-amino-1-methyl-2-oxo-ethyl)-2-[2-
(3,4-difluoro-2-methyl-phenoxy)-3-quinolyl]-6-
methyl-4-oxo-1H-pyridine-3-carboxylate 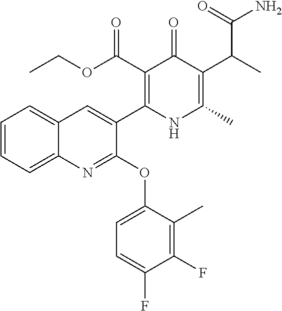
ethyl (S)-5-(2-amino-1-methyl-2-oxo-ethyl)-2-[2-
(3,4-difluoro-2-methyl-phenoxy)-3-quinolyl]-6-
methyl-4-oxo-1H-pyridine-3-carboxylate 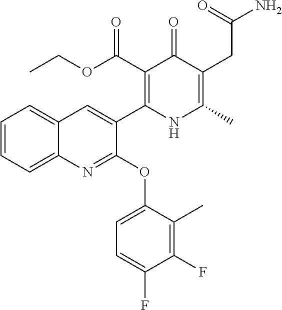
ethyl (R)-5-(2-amino-1-methyl-2-oxo-ethyl)-2-[2-
(3,4-difluoro-2-methyl-phenoxy)-3-quinolyl]-6-
methyl-4-oxo-1H-pyridine-3-carboxylate 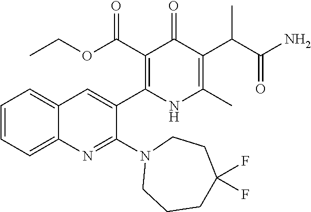 [SMILES: CCOC(=O)c1c(-c2cc3ccccc3nc2N2CCCC(F)(F)CC2)[nH]c(C)c(C(C)C(N)=O)c1=O]
ethyl 5-(2-amino-1-methyl-2-oxo-ethyl)-2-[2-
(4,4-difluoroazepan-1-yl)-3-quinolyl]-6-methyl-4-
oxo-1H-pyridine-3-carboxylate 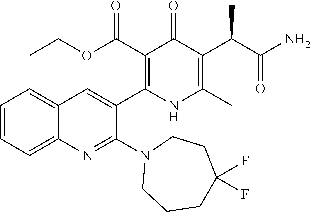 [SMILES: CCOC(=O)c1c(-c2cc3ccccc3nc2N2CCCC(F)(F)CC2)[nH]c(C)c([C@@H](C)C(N)=O)c1=O]
ethyl (R)-5-(2-amino-1-methyl-2-oxo-ethyl)-2-[2-
(4,4-difluoroazepan-1-yl)-3-quinolyl]-6-methyl-4-
oxo-1H-pyridine-3-carboxylate 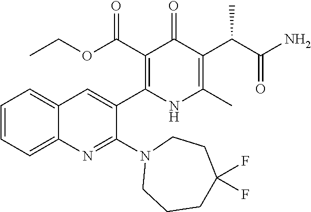 [SMILES: CCOC(=O)c1c(-c2cc3ccccc3nc2N2CCCC(F)(F)CC2)[nH]c(C)c([C@H](C)C(N)=O)c1=O]
ethyl (S)-5-(2-amino-1-methyl-2-oxo-ethyl)-2-[2-
(4,4-difluoroazepan-1-yl)-3-quinolyl]-6-methyl-4-
oxo-1H-pyridine-3-carboxylate 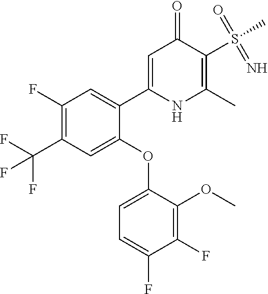 [SMILES: COc1c(Oc2cc(C(F)(F)F)c(F)cc2-c2cc(=O)c([S@](C)(=N)=O)c(C)[nH]2)ccc(F)c1F]
6-[2-(3,4-difluoro-2-methoxy-phenoxy)-5-fluoro-
4-(trifluoromethyl)phenyl]-2-methyl-3-
(methylsulfonimidoyl)-1H-pyridin-4-one 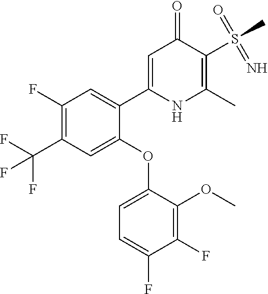 [SMILES: COc1c(Oc2cc(C(F)(F)F)c(F)cc2-c2cc(=O)c([S@@](C)(=N)=O)c(C)[nH]2)ccc(F)c1F]
(S)-6-[2-(3,4-difluoro-2-methoxy-phenoxy)-5-
fluoro-4-(trifluoromethyl)phenyl]-2-methyl-3-
(methylsulfonimidoyl)-1H-pyridin-4-one 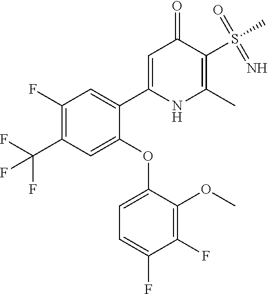 [SMILES: COc1c(Oc2cc(C(F)(F)F)c(F)cc2-c2cc(=O)c([S@](C)(=N)=O)c(C)[nH]2)ccc(F)c1F]
(R)-6-[2-(3,4-difluoro-2-methoxy-phenoxy)-5-
fluoro-4-(trifluoromethyl)phenyl]-2-methyl-3-
(methylsulfonimidoyl)-1H-pyridin-4-one 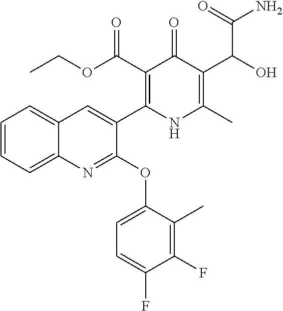 [SMILES: CCOC(=O)c1c(-c2cc3ccccc3nc2Oc2ccc(F)c(F)c2C)[nH]c(C)c(C(O)C(N)=O)c1=O]
ethyl 5-(2-amino-1-hydroxy-2-oxo-ethyl)-2-[2-
(3,4-difluoro-2-methyl-phenoxy)-3-quinolyl]-6-
methyl-4-oxo-1H-pyridine-3-carboxylate 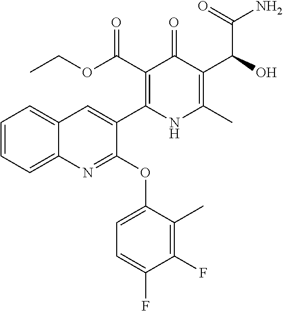 [SMILES: CCOC(=O)c1c(-c2cc3ccccc3nc2Oc2ccc(F)c(F)c2C)[nH]c(C)c([C@H](O)C(N)=O)c1=O]
ethyl (S)-5-(2-amino-1-hydroxy-2-oxo-ethyl)-2-
[2-(3,4-difluoro-2-methyl-phenoxy)-3-quinolyl]-
6-methyl-4-oxo-1H-pyridine-3-carboxylate 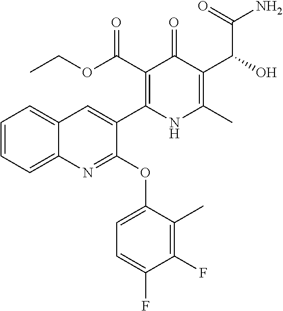 [SMILES: CCOC(=O)c1c(-c2cc3ccccc3nc2Oc2ccc(F)c(F)c2C)[nH]c(C)c([C@@H](O)C(N)=O)c1=O]
ethyl (R)-5-(2-amino-1-hydroxy-2-oxo-ethyl)-2-
[2-(3,4-difluoro-2-methyl-phenoxy)-3-quinolyl]-
6-methyl-4-oxo-1H-pyridine-3-carboxylate 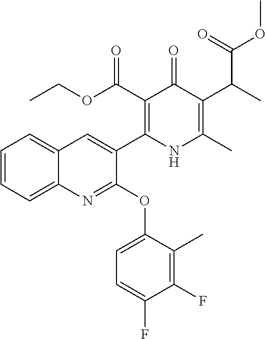 [SMILES: CCOC(=O)c1c(-c2cc3ccccc3nc2Oc2ccc(F)c(F)c2C)[nH]c(C)c(C(C)C(=O)OC)c1=O]
ethyl 2-[2-(3,4-difluoro-2-methyl-phenoxy)-3-
quinolyl]-5-(2-methoxy-1-methyl-2-oxo-ethyl)-6-
methyl-4-oxo-1H-pyridine-3-carboxylate 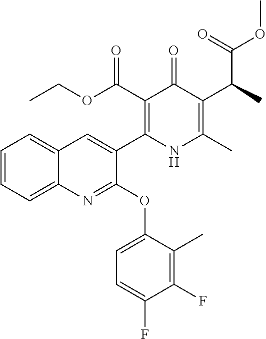 [SMILES: CCOC(=O)c1c(-c2cc3ccccc3nc2Oc2ccc(F)c(F)c2C)[nH]c(C)c([C@H](C)C(=O)OC)c1=O]
ethyl (S)-2-[2-(3,4-difluoro-2-methyl-phenoxy)-3-
quinolyl]-5-(2-methoxy-1-methyl-2-oxo-ethyl)-6-
methyl-4-oxo-1H-pyridine-3-carboxylate 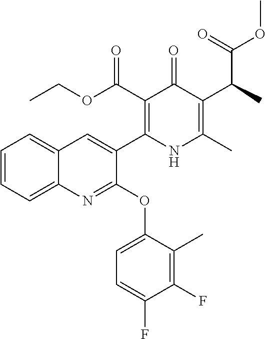 [SMILES: CCOC(=O)c1c(-c2cc3ccccc3nc2Oc2ccc(F)c(F)c2C)[nH]c(C)c([C@H](C)C(=O)OC)c1=O]
ethyl (R)-2-[2-(3,4-difluoro-2-methyl-phenoxy)-3-
quinolyl]-5-(2-methoxy-1-methyl-2-oxo-ethyl)-6-
methyl-4-oxo-1H-pyridine-3-carboxylate 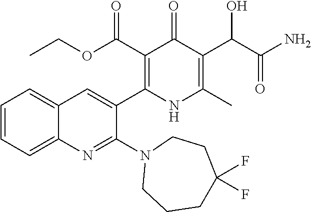 [SMILES: CCOC(=O)c1c(-c2cc3ccccc3nc2N2CCCC(F)(F)CC2)[nH]c(C)c(C(O)C(N)=O)c1=O]
ethyl 5-(2-amino-1-hydroxy-2-oxo-ethyl)-2-[2-
(4,4-difluoroazepan-1-yl)-3-quinolyl]-6-methyl-4-
oxo-1H-pyridine-3-carboxylate 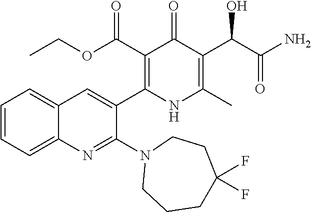 [SMILES: CCOC(=O)c1c(-c2cc3ccccc3nc2N2CCCC(F)(F)CC2)[nH]c(C)c([C@@H](O)C(N)=O)c1=O]
ethyl (R)-5-(2-amino-1-hydroxy-2-oxo-ethyl)-2-
[2-(4,4-difluoroazepan-1-yl)-3-quinolyl]-6-
methyl-4-oxo-1H-pyridine-3-carboxylate 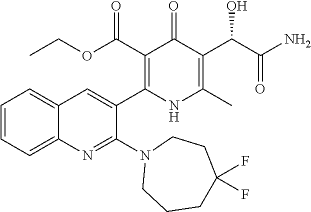 [SMILES: CCOC(=O)c1c(-c2cc3ccccc3nc2N2CCCC(F)(F)CC2)[nH]c(C)c([C@H](O)C(N)=O)c1=O]
ethyl (S)-5-(2-amino-1-hydroxy-2-oxo-ethyl)-2-
[2-(4,4-difluoroazepan-1-yl)-3-quinolyl]-6-
methyl-4-oxo-1H-pyridine-3-carboxylate 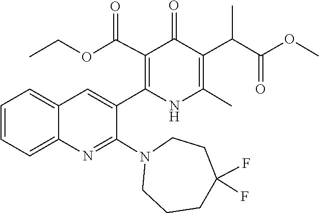 [SMILES: CCOC(=O)c1c(-c2cc3ccccc3nc2N2CCCC(F)(F)CC2)[nH]c(C)c(C(C)C(=O)OC)c1=O]
ethyl 2-[2-(4,4-difluoroazepan-1-yl)-3-quinolyl]-
5-(2-methoxy-1-methyl-2-oxo-ethyl)-6-methyl-4-
oxo-1H-pyridine-3-carboxylate 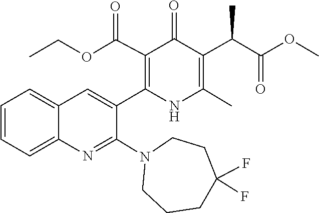 [SMILES: CCOC(=O)c1c(-c2cc3ccccc3nc2N2CCCC(F)(F)CC2)[nH]c(C)c([C@@H](C)C(=O)OC)c1=O]
ethyl (R)-2-[2-(4,4-difluoroazepan-1-yl)-3-
quinolyl]-5-(2-methoxy-1-methyl-2-oxo-ethyl)-6-
methyl-4-oxo-1H-pyridine-3-carboxylate 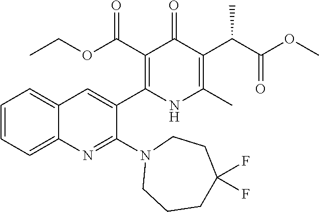 [SMILES: CCOC(=O)c1c(-c2cc3ccccc3nc2N2CCCC(F)(F)CC2)[nH]c(C)c([C@H](C)C(=O)OC)c1=O]
ethyl (S)-2-[2-(4,4-difluoroazepan-1-yl)-3-
quinolyl]-5-(2-methoxy-1-methyl-2-oxo-ethyl)-6-
methyl-4-oxo-1H-pyridine-3-carboxylate 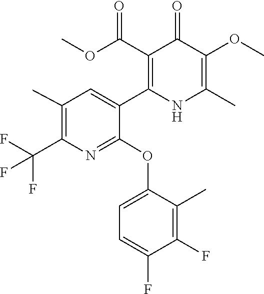 [SMILES: COC(=O)c1c(-c2cc(C)c(C(F)(F)F)nc2Oc2ccc(F)c(F)c2C)[nH]c(C)c(OC)c1=O]
methyl 2-[2-(3,4-difluoro-2-methyl-phenoxy)-5-
methyl-6-(trifluoromethyl)-3-pyridyl]-5-methoxy-
6-methyl-4-oxo-1H-pyridine-3-carboxylate 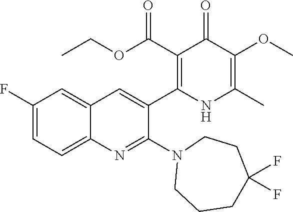 [SMILES: CCOC(=O)c1c(-c2cc3cc(F)ccc3nc2N2CCCC(F)(F)CC2)[nH]c(C)c(OC)c1=O]
ethyl 2-[2-(4,4-difluoroazepan-1-yl)-6-fluoro-3-
quinolyl]-5-methoxy-6-methyl-4-oxo-1H-
pyridine-3-carboxylate 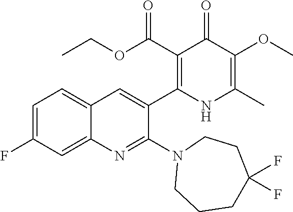 [SMILES: CCOC(=O)c1c(-c2cc3ccc(F)cc3nc2N2CCCC(F)(F)CC2)[nH]c(C)c(OC)c1=O]
ethyl 2-[2-(4,4-difluoroazepan-1-yl)-7-fluoro-3-
quinolyl]-5-methoxy-6-methyl-4-oxo-1H-
pyridine-3-carboxylate 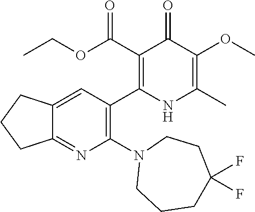 [SMILES: CCOC(=O)c1c(-c2cc3c(nc2N2CCCC(F)(F)CC2)CCC3)[nH]c(C)c(OC)c1=O]
ethyl 2-[2-(4,4-difluoroazepan-1-yl)-6,7-dihydro-
5H-cyclopenta[b]pyridin-3-yl]-5-methoxy-6-
methyl-4-oxo-1H-pyridine-3-carboxylate 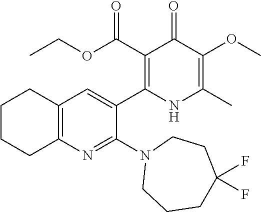 [SMILES: CCOC(=O)c1c(-c2cc3c(nc2N2CCCC(F)(F)CC2)CCCC3)[nH]c(C)c(OC)c1=O]
ethyl 2-[2-(4,4-difluoroazepan-1-yl)-5,6,7,8-
tetrahydroquinolin-3-yl]-5-methoxy-6-methyl-4-
oxo-1H-pyridine-3-carboxylate 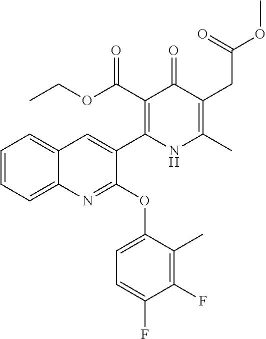 [SMILES: CCOC(=O)c1c(-c2cc3ccccc3nc2Oc2ccc(F)c(F)c2C)[nH]c(C)c(CC(=O)OC)c1=O]
ethyl 2-[2-(3,4-difluoro-2-methyl-phenoxy)-3-
quinolyl]-5-(2-methoxy-2-oxo-ethyl)-6-methyl-
4-oxo-1H-pyridine-3-carboxylate 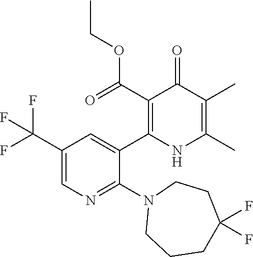 [SMILES: CCOC(=O)c1c(-c2cc(C(F)(F)F)cnc2N2CCCC(F)(F)CC2)[nH]c(C)c(C)c1=O]
ethyl 2-[2-(4,4-difluoroazepan-1-yl)-5-
(trifluoromethyl)-3-pyridyl]-5,6-dimethyl-4-oxo-
1H-pyridine-3-carboxylate 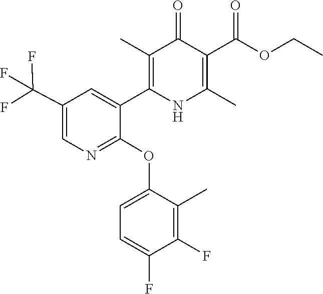 [SMILES: CCOC(=O)c1c(C)[nH]c(-c2cc(C(F)(F)F)cnc2Oc2ccc(F)c(F)c2C)c(C)c1=O]
ethyl 6-[2-(3,4-difluoro-2-methyl-phenoxy)-5-
(trifluoromethyl)-3-pyridyl]-2,5-dimethyl-4-oxo-
1H-pyridine-3-carboxylate 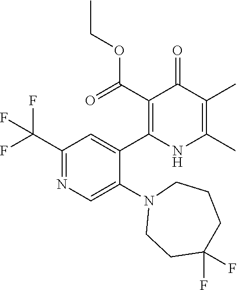 [SMILES: CCOC(=O)c1c(-c2cc(C(F)(F)F)ncc2N2CCCC(F)(F)CC2)[nH]c(C)c(C)c1=O]
ethyl 2-[5-(4,4-difluoroazepan-1-yl)-2-
(trifluoromethyl)-4-pyridyl]-5,6-dimethyl-4-oxo-
1H-pyridine-3-carboxylate 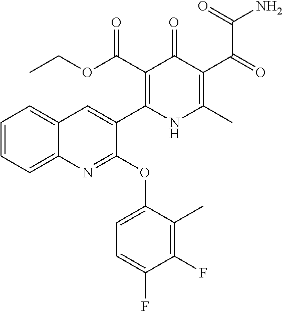 [SMILES: CCOC(=O)c1c(-c2cc3ccccc3nc2Oc2ccc(F)c(F)c2C)[nH]c(C)c(C(=O)C(N)=O)c1=O]
ethyl 2-[2-(3,4-difluoro-2-methyl-phenoxy)-3-
quinolyl]-6-methyl-5-oxamoyl-4-oxo-1H-
pyridine-3-carboxylate 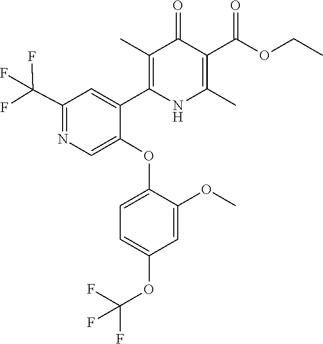 [SMILES: CCOC(=O)c1c(C)[nH]c(-c2cc(C(F)(F)F)ncc2Oc2ccc(OC(F)(F)F)cc2OC)c(C)c1=O]
ethyl 6-[5-[2-methoxy-4-
(trifluoromethoxy)phenoxy]-2-(trifluoromethyl)-
4-pyridyl]-2,5-dimethyl-4-oxo-1H-pyridine-3-
carboxylate 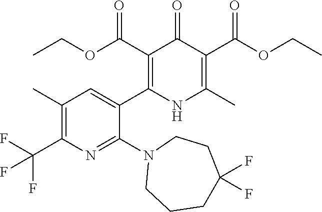 [SMILES: CCOC(=O)c1c(C)[nH]c(-c2cc(C)c(C(F)(F)F)nc2N2CCCC(F)(F)CC2)c(C(=O)OCC)c1=O]
diethyl 2-[2-(4,4-difluoroazepan-1-yl)-5-methyl-
6-(trifluoromethyl)-3-pyridyl]-6-methyl-4-oxo-
1H-pyridine-3,5-carboxylate 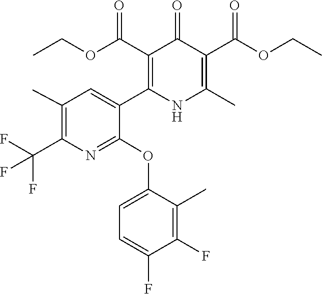 [SMILES: CCOC(=O)c1c(C)[nH]c(-c2cc(C)c(C(F)(F)F)nc2Oc2ccc(F)c(F)c2C)c(C(=O)OCC)c1=O]
diethyl 2-[2-(3,4-difluoro-2-methyl-phenoxy)-5-
methyl-6-(trifluoromethyl)-3-pyridyl]-6-methyl-4-
oxo-1H-pyridine-3,5-carboxylate 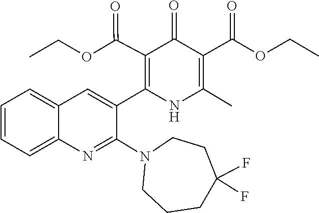 [SMILES: CCOC(=O)c1c(C)[nH]c(-c2cc3ccccc3nc2N2CCCC(F)(F)CC2)c(C(=O)OCC)c1=O]
diethyl 2-[2-(4,4-difluoroazepan-1-yl)-3-
quinolyl]-6-methyl-4-oxo-1H-pyridine-3,5-
dicarboxylate 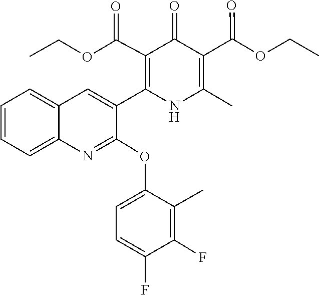 [SMILES: CCOC(=O)c1c(C)[nH]c(-c2cc3ccccc3nc2Oc2ccc(F)c(F)c2C)c(C(=O)OCC)c1=O]
diethyl 2-[2-(3,4-difluoro-2-methyl-phenoxy)-3-
quinolyl]-6-methyl-4-oxo-1H-pyridine-3,5-
dicarboxylate 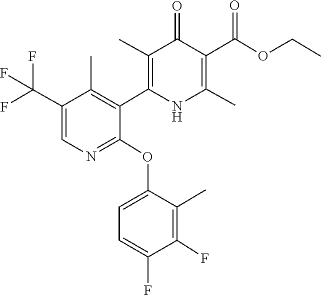 [SMILES: CCOC(=O)c1c(C)[nH]c(-c2c(Oc3ccc(F)c(F)c3C)ncc(C(F)(F)F)c2C)c(C)c1=O]
diethyl 6-[2-(3,4-difluoro-2-methyl-phenoxy)-4-
methyl-5-(trifluoromethyl)-3-pyridyl]-2,5-
dimethyl-5-oxo-1H-pyridine-3-carboxylate 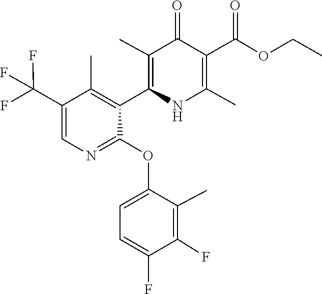
ethyl 6-[2-(3,4-difluoro-2-methyl-phenoxy)-4-
methyl-5-(trifluoromethyl)-3-pyridyl]-2,5-
dimethyl-4-oxo-1H-pyridine-3-carboxylate 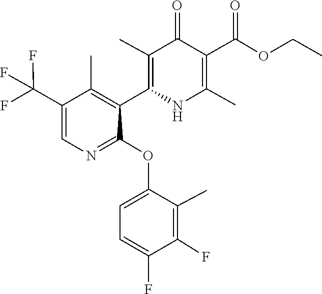
ethyl 6-[2-(3,4-difluoro-2-methyl-phenoxy)-4-
methyl-5-(trifluoromethyl)-3-pyridyl]-2,5-
dimethyl-4-oxo-1H-pyridine-3-carboxylate 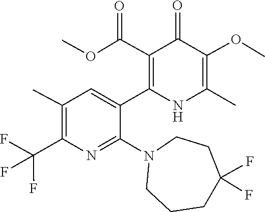 [SMILES: COC(=O)c1c(-c2cc(C)c(C(F)(F)F)nc2N2CCCC(F)(F)CC2)[nH]c(C)c(OC)c1=O]
methyl 2-[2-(4,4-difluoroazepan-1-yl)-5-methyl-
6-(trifluoromethyl)-3-pyridyl]-5-methoxy-6-
methyl-4-oxo-1H-pyridine-3-carboxylate 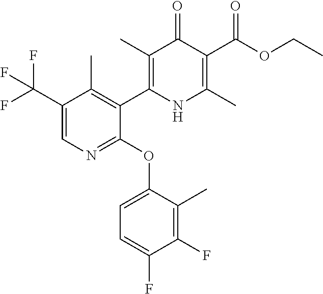 [SMILES: CCOC(=O)c1c(C)[nH]c(-c2c(Oc3ccc(F)c(F)c3C)ncc(C(F)(F)F)c2C)c(C)c1=O]
ethyl 6-[2-(3,4-difluoro-2-methyl-phenoxy)-4-
methyl-5-(trifluoromethyl)-3-pyridyl]-2,5-
dimethyl-4-oxo-1H-pyridine-3-carboxylate 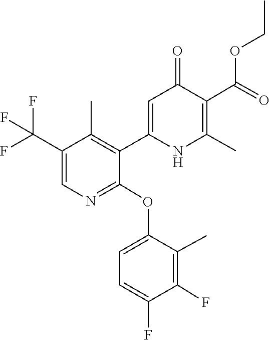 [SMILES: CCOC(=O)c1c(C)[nH]c(-c2c(Oc3ccc(F)c(F)c3C)ncc(C(F)(F)F)c2C)cc1=O]
ethyl 6-[2-(3,4-difluoro-2-methyl-phenoxy)-4-
methyl-5-(trifluoromethyl)-3-pyridyl]-2-methyl-4-
oxo-1H-pyridine-3-carboxylate 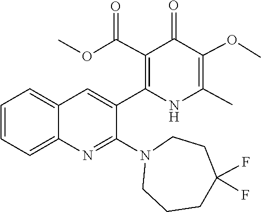 [SMILES: COC(=O)c1c(-c2cc3ccccc3nc2N2CCCC(F)(F)CC2)[nH]c(C)c(OC)c1=O]
methyl 2-[2-(4,4-difluoroazepan-1-yl)-3-
quinolyl]-5-methoxy-6-methyl-4-oxo-1H-
pyridine-3-carboxylate 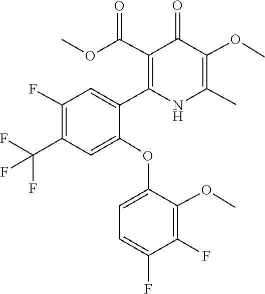 [SMILES: COC(=O)c1c(-c2cc(F)c(C(F)(F)F)cc2Oc2ccc(F)c(F)c2OC)[nH]c(C)c(OC)c1=O]
methyl 2-[2-(3,4-difluoro-2-methoxy-phenoxy)-5-
fluoro-4-(trifluoromethyl)phenyl]-5-methoxy-6-
methyl-4-oxo-1H-pyridine-3-carboxylate 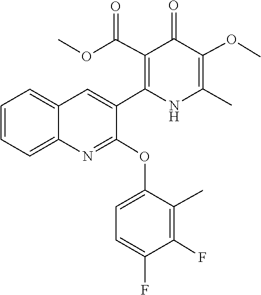 [SMILES: COC(=O)c1c(-c2cc3ccccc3nc2Oc2ccc(F)c(F)c2C)[nH]c(C)c(OC)c1=O]
methyl 2-[2-(3,4-difluoro-2-methyl-phenoxy)-3-
quinolyl]-5-methoxy-6-methyl-4-oxo-1H-
pyridine-3-carboxylate 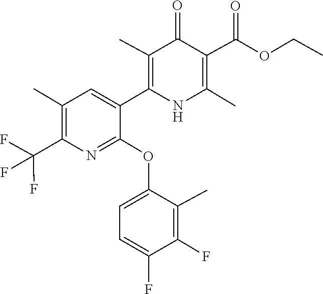 [SMILES: CCOC(=O)c1c(C)[nH]c(-c2cc(C)c(C(F)(F)F)nc2Oc2ccc(F)c(F)c2C)c(C)c1=O]
ethyl 6-[2-(3,4-difluoro-2-methyl-phenoxy)-5-
methyl-6-(trifluoromethyl)-3-pyridyl]-2,5-
dimethyl-4-oxo-1H-pyridine-3-carboxylate 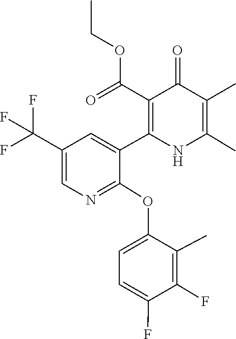 [SMILES: CCOC(=O)c1c(-c2cc(C(F)(F)F)cnc2Oc2ccc(F)c(F)c2C)[nH]c(C)c(C)c1=O]
ethyl 2-[2-(3,4-difluoro-2-methyl-phenoxy)-5-
(trifluoromethyl)-3-pyridyl]-5,6-dimethyl-4-oxo-
1H-pyridine-3-carboxylate 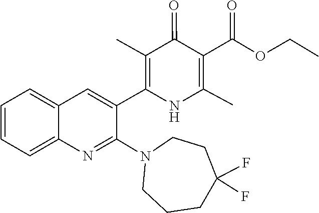 [SMILES: CCOC(=O)c1c(C)[nH]c(-c2cc3ccccc3nc2N2CCCC(F)(F)CC2)c(C)c1=O]
ethyl 6-[2-(4,4-difluoroazepan-1-yl)-3-quinolyl-
2,5-dimethyl-4-oxo-1H-pyridine-3-carboxylate 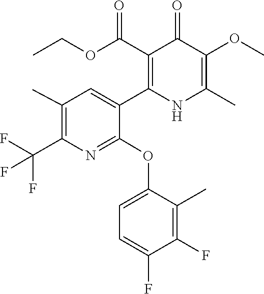 [SMILES: CCOC(=O)c1c(-c2cc(C)c(C(F)(F)F)nc2Oc2ccc(F)c(F)c2C)[nH]c(C)c(OC)c1=O]
ethyl 2-[2-(3,4-difluoro-2-methyl-phenoxy)-5-
methyl-6-(trifluoromethyl)-3-pyridyl]-5-methoxy-
6-methyl-4-oxo-1H-pyridine-3-carboxylate 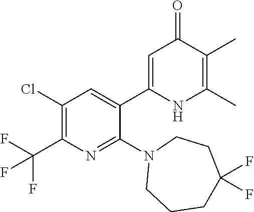 [SMILES: Cc1[nH]c(-c2cc(Cl)c(C(F)(F)F)nc2N2CCCC(F)(F)CC2)cc(=O)c1C]
6-[5-chloro-2-(4,4-difluoroazepan-1-yl)-6-
(trifluoromethyl)-3-pyridyl]-2,3-dimethyl-1H-
pyridin-4-one 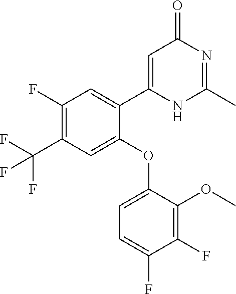 [SMILES: COc1c(Oc2cc(C(F)(F)F)c(F)cc2-c2cc(=O)nc(C)[nH]2)ccc(F)c1F]
6-[2-(3,4-difluoro-2-methoxy-phenoxy)-5-fluoro-
4-(trifluoromethyl)phenyl]-2-methyl-1H-
pyrimidin-4-one 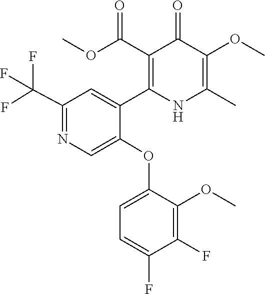 [SMILES: COC(=O)c1c(-c2cc(C(F)(F)F)ncc2Oc2ccc(F)c(F)c2OC)[nH]c(C)c(OC)c1=O]
methyl 2-[5-(3,4-difluoro-2-methoxy-phenoxy)-2-
(trifluoromethyl)-4-pyridyl]-5-methoxy-6-methyl-
4-oxo-1H-pyridine-3-carboxylate 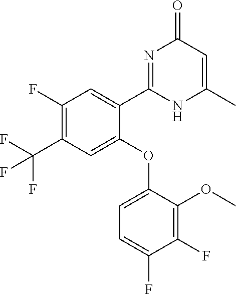 [SMILES: COc1c(Oc2cc(C(F)(F)F)c(F)cc2-c2nc(=O)cc(C)[nH]2)ccc(F)c1F]
2-[2-(3,4-difluoro-2-methoxy-phenoxy)-5-fluoro-
4-(trifluoromethyl)phenyl]-6-methyl-1H-
pyrimidin-4-one 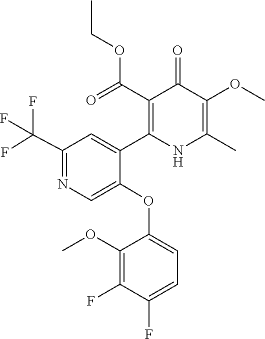 [SMILES: CCOC(=O)c1c(-c2cc(C(F)(F)F)ncc2Oc2ccc(F)c(F)c2OC)[nH]c(C)c(OC)c1=O]
ethyl 2-[5-(3,4-difluoro-2-methoxy-phenoxy)-2-
(trifluoromethyl)-4-pyridyl]-5-methoxy-6-methyl-
4-oxo-1H-pyridine-3-carboxylate 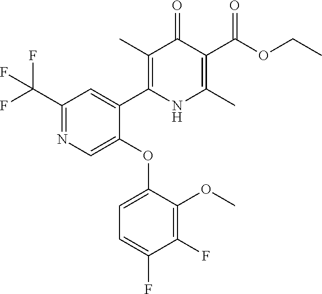 [SMILES: CCOC(=O)c1c(C)[nH]c(-c2cc(C(F)(F)F)ncc2Oc2ccc(F)c(F)c2OC)c(C)c1=O]
ethyl 6-[5-(3,4-difluoro-2-methoxy-phenoxy)-2-
(trifluoromethyl)-4-pyridyl]-2,5-dimethyl-4-oxo-
1H-pyridine-3-carboxylate 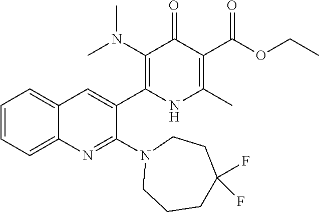 [SMILES: CCOC(=O)c1c(C)[nH]c(-c2cc3ccccc3nc2N2CCCC(F)(F)CC2)c(N(C)C)c1=O]
ethyl 6-[2-(4,4-difluoroazepan-1-yl)-3-quinolyl-
5-(dimethylamino)-2-methyl-4-oxo-1H-pyridine-
3-carboxylate 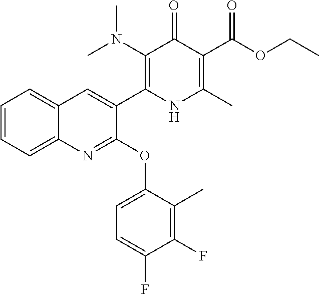 [SMILES: CCOC(=O)c1c(C)[nH]c(-c2cc3ccccc3nc2Oc2ccc(F)c(F)c2C)c(N(C)C)c1=O]
ethyl 6-[2-(3,4-difluoro-2-methyl-phenoxy)-3-
quinolyl]-5-(dimethylamino)-2-methyl-4-oxo-1H-
pyridine-3-carboxylate 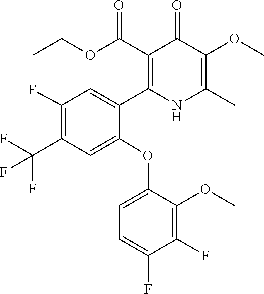 [SMILES: CCOC(=O)c1c(-c2cc(F)c(C(F)(F)F)cc2Oc2ccc(F)c(F)c2OC)[nH]c(C)c(OC)c1=O]
ethyl 2-[2-(3,4-difluoro-2-methoxy-phenoxy)-5-
fluoro-4-(trifluoromethyl)phenyl]-5-methoxy-6-
methyl-4-oxo-1H-pyridine-carboxylate 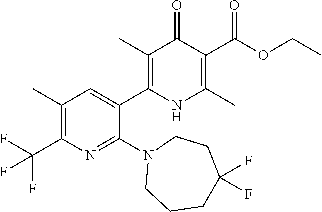 [SMILES: CCOC(=O)c1c(C)[nH]c(-c2cc(C)c(C(F)(F)F)nc2N2CCCC(F)(F)CC2)c(C)c1=O]
ethyl 6-[2-(4,4-difluoroazepan-1-yl)-5-methyl-6-
(trifluoromethyl)-3-pyridyl]-2,5-dimethyl-4-oxo-
1H-pyridine-3-carboxylate 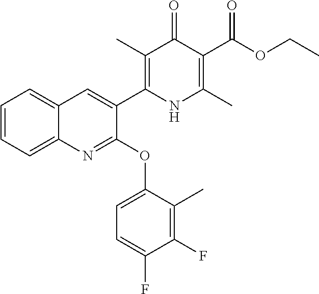 [SMILES: CCOC(=O)c1c(C)[nH]c(-c2cc3ccccc3nc2Oc2ccc(F)c(F)c2C)c(C)c1=O]
ethyl 6-[2-(3,4-difluoro-2-methyl-phenoxy)-3-
quinolyl]-2,5-dimethyl-4-oxo-1H-pyridine-3-
carboxylate 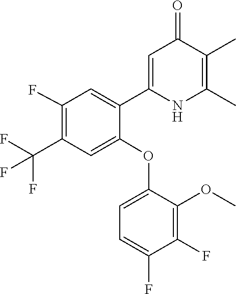 [SMILES: COc1c(Oc2cc(C(F)(F)F)c(F)cc2-c2cc(=O)c(C)c(C)[nH]2)ccc(F)c1F]
6-[2-(3,4-difluoro-2-methoxy-phenoxy)-5-fluoro-
4-(trifluoromethyl)phenyl]-2,3-dimethyl-1H-
pyridine-4-one 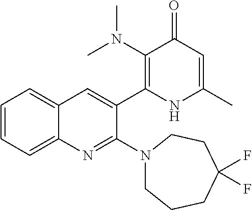 [SMILES: Cc1cc(=O)c(N(C)C)c(-c2cc3ccccc3nc2N2CCCC(F)(F)CC2)[nH]1]
2-[2-(4,4-difluoroazepan-1-yl)-3-quinolyl]-3-
(dimethylamino)-6-methyl-1H-pyridin-4-one 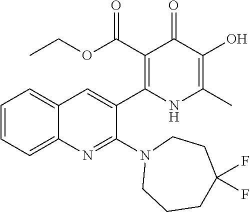 [SMILES: CCOC(=O)c1c(-c2cc3ccccc3nc2N2CCCC(F)(F)CC2)[nH]c(C)c(O)c1=O]
ethyl 2-[2-(4,4-difluoroazepan-1-yl)-3-quinolyl]-
5-hydroxy-6-methyl-4-oxo-1H-pyridine-3-
carboxylate 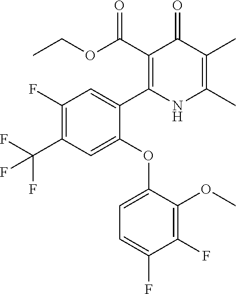 [SMILES: CCOC(=O)c1c(-c2cc(F)c(C(F)(F)F)cc2Oc2ccc(F)c(F)c2OC)[nH]c(C)c(C)c1=O]
ethyl 2-[2-(3,4-difluoro-2-methyl-phenoxy)-5-
methyl-6-(trifluoromethyl)-3-pyridyl]-5,6-
dimethyl-4-oxo-1H-pyridine-3-carboxylate 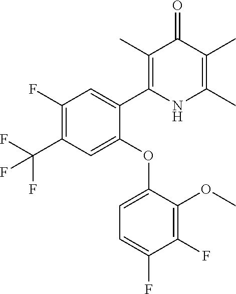 [SMILES: COc1c(Oc2cc(C(F)(F)F)c(F)cc2-c2[nH]c(C)c(C)c(=O)c2C)ccc(F)c1F]
2-[2-(3,4-difluoro-2-methoxy-phenoxy)-5-fluoro-
4-(trifluoromethyl)phenyl]-3,5,6-trimethyl-1H-
pyridine-4-one 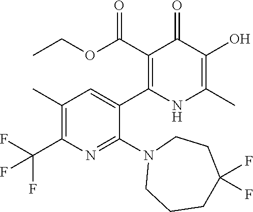 [SMILES: CCOC(=O)c1c(-c2cc(C)c(C(F)(F)F)nc2N2CCCC(F)(F)CC2)[nH]c(C)c(O)c1=O]
ethyl 2-[2-(4,4-difluoroazepan-1-yl)-5-methyl-6-
(trifluoromethyl)-3-pyridyl]-5-hydroxy-6-methyl-
4-oxo-1H-pyridine-3-carboxylate 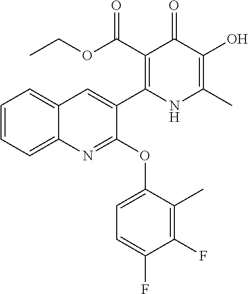 [SMILES: CCOC(=O)c1c(-c2cc3ccccc3nc2Oc2ccc(F)c(F)c2C)[nH]c(C)c(O)c1=O]
ethyl 2-[2-(3,4-difluoro-2-methyl-phenoxy)-3-
quinolyl]-5-hydroxy-6-methyl-4-oxo-1H-
pyridine-3-carboxylate 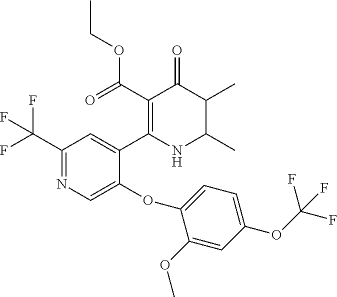 [SMILES: CCOC(=O)C1=C(c2cc(C(F)(F)F)ncc2Oc2ccc(OC(F)(F)F)cc2OC)NC(C)C(C)C1=O]
ethyl 2-[5-(2-methoxy)-4-
(trifluoromethoxy)phenoxy]-2-(trifluoromethyl)-
4-pyridyl]-5,6-dimethyl-4-oxo-1H-pyridine-
3-carboxylate 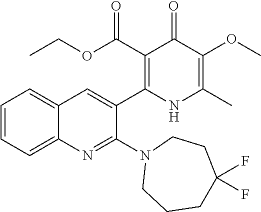 [SMILES: CCOC(=O)c1c(-c2cc3ccccc3nc2N2CCCC(F)(F)CC2)[nH]c(C)c(OC)c1=O]
ethyl 2-[2-(4,4-difluoroazepan-1-yl)-3-quinolyl]-
5-methoxy-6-methyl-4-oxo-1H-pyridine-3-
carboxylate 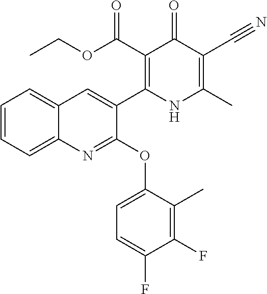 [SMILES: CCOC(=O)c1c(-c2cc3ccccc3nc2Oc2ccc(F)c(F)c2C)[nH]c(C)c(C#N)c1=O]
ethyl 5-cyano-2-[2-(3,4-difluoro-2-methyl-
phenoxy)-3-quinolyl]-6-methyl-4-oxo-1H-
pyridine-3-carboxylate 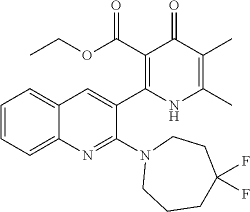 [SMILES: CCOC(=O)c1c(-c2cc3ccccc3nc2N2CCCC(F)(F)CC2)[nH]c(C)c(C)c1=O]
ethyl 2-[2-(4,4-difluoroazepan-1-yl)-3-quinolyl]-
5,6-dimethyl-4-oxo-1H-pyridine-3-carboxylate 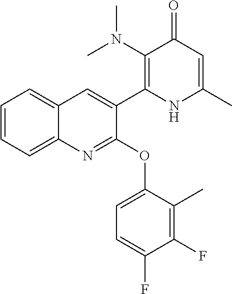 [SMILES: Cc1cc(=O)c(N(C)C)c(-c2cc3ccccc3nc2Oc2ccc(F)c(F)c2C)[nH]1]
2-[2-(3,4-difluoro-2-methyl-phenoxy)-3-
quinolyl]-3-(dimethylamino)-6-methyl-1H-
pyridin-4-one 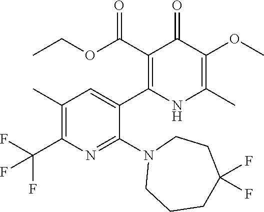 [SMILES: CCOC(=O)c1c(-c2cc(C)c(C(F)(F)F)nc2N2CCCC(F)(F)CC2)[nH]c(C)c(OC)c1=O]
ethyl 2-[2-(4,4-difluoroazepan-1-yl)-5-methyl-6-
(trifluoromethyl)-3-pyridyl]-5-methoxy-6-methyl-
4-oxo-1H-pyridine-3-carboxylate 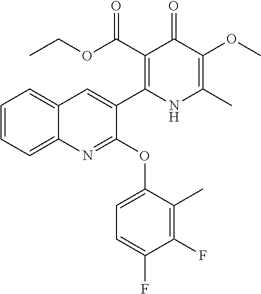 [SMILES: CCOC(=O)c1c(-c2cc3ccccc3nc2Oc2ccc(F)c(F)c2C)[nH]c(C)c(OC)c1=O]
ethyl 2-[2-(3,4-difluoro-2-methyl-phenoxy)-3-
quinolyl]-5-methoxy-6-methyl-4-oxo-1H-
pyridine-3-carboxylate 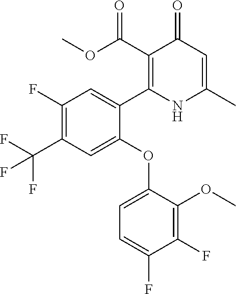 [SMILES: COC(=O)c1c(-c2cc(F)c(C(F)(F)F)cc2Oc2ccc(F)c(F)c2OC)[nH]c(C)cc1=O]
methyl 2-[2-(3,4-difluoro-2-methoxy-phenoxy)-5-
fluoro-4-(trifluoromethyl)phenyl]-6-methyl-4-
oxo-1H-pyridine-3-carboxylate 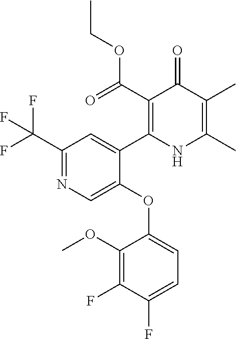 [SMILES: CCOC(=O)c1c(-c2cc(C(F)(F)F)ncc2Oc2ccc(F)c(F)c2OC)[nH]c(C)c(C)c1=O]
ethyl 2-[5-(3,4-difluoro-2-methoxy-phenoxy)-2-
(trifluoromethyl)-4-pyridyl]-5,6-dimethyl-4-oxo-
1H-pyridine-3-carboxylate 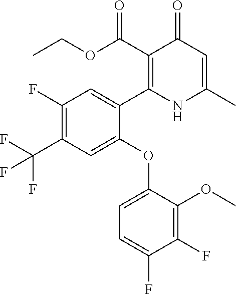 [SMILES: CCOC(=O)c1c(-c2cc(F)c(C(F)(F)F)cc2Oc2ccc(F)c(F)c2OC)[nH]c(C)cc1=O]
ethyl 2-[2-(3,4-difluoro-2-methoxy-phenoxy)-5-
fluoro-4-(trifluoromethyl)phenyl]-6-methyl-4-
oxo-1H-pyridine-3-carboxylate 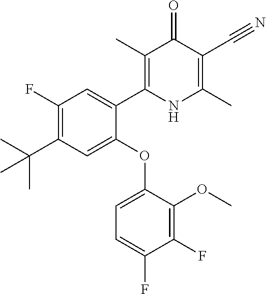 [SMILES: COc1c(Oc2cc(C(C)(C)C)c(F)cc2-c2[nH]c(C)c(C#N)c(=O)c2C)ccc(F)c1F]
6-[2-(3,4-difluoro-2-methoxy-phenoxy)-5-fluoro-
4-(trifluoromethyl)phenyl]-2,5-dimethyl-4-oxo-
1H-pyridine-3-carboxylate 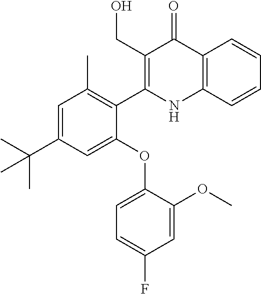 [SMILES: COc1cc(F)ccc1Oc1cc(C(C)(C)C)cc(C)c1-c1[nH]c2ccccc2c(=O)c1CO]
ethyl 2-[2-(3,4-difluoro-2-methyl-phenoxy)-4-
methyl-3-quinolyl]-5,6-dimethyl-4-oxo-1H-
pyridine-3-carboxylate 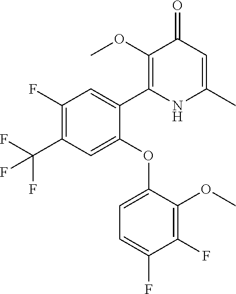 [SMILES: COc1c(Oc2cc(C(F)(F)F)c(F)cc2-c2[nH]c(C)cc(=O)c2OC)ccc(F)c1F]
2-[2-(3,4-difluoro-2-methoxy-phenoxy)-5-fluoro-
4-(trifluoromethyl)phenyl]-3-methoxy-6-methyl-
1H-pyridin-4-one 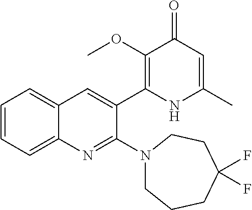 [SMILES: COc1c(-c2cc3ccccc3nc2N2CCCC(F)(F)CC2)[nH]c(C)cc1=O]
2-[2-(4,4-difluoroazepan-1-yl)-3-quinolyl]-3-
methoxy-6-methyl-1H-pyridin-4-one 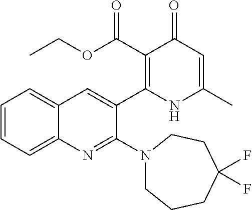 [SMILES: CCOC(=O)c1c(-c2cc3ccccc3nc2N2CCCC(F)(F)CC2)[nH]c(C)cc1=O]
ethyl 2-[2-(4,4-difluoroazepan-1-yl)-3-quinolyl]-
6-methyl-4-oxo-1H-pyridine-3-carboxylate 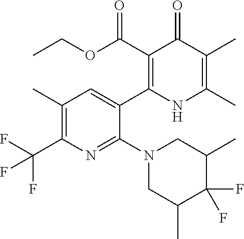 [SMILES: CCOC(=O)c1c(-c2cc(C)c(C(F)(F)F)nc2N2CC(C)C(F)(F)C(C)C2)[nH]c(C)c(C)c1=O]
ethyl 2-[2-(4,4-difluoro-3,5-dimethyl-1-
piperidyl]-5-methyl-6-((rifluoromethyl)-3-
pyridyl]-5,6-dimethyl-4-oxo-1H-pyridine-3-
carboxylate 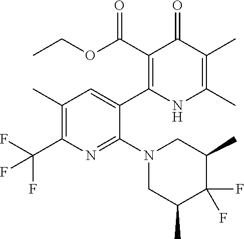 [SMILES: CCOC(=O)c1c(-c2cc(C)c(C(F)(F)F)nc2N2C[C@@H](C)C(F)(F)[C@@H](C)C2)[nH]c(C)c(C)c1=O]
ethyl 2-[2-[(3R,5S)-4,4-difluoro-3,5-dimethyl-1-
piperidyl]-5-methyl-6-(trifluoromethyl)-3-
pyridyl]-5,6-dimethyl-4-oxo-1H-pyridine-3-
carboxylate 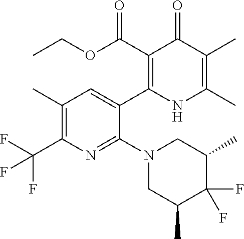 [SMILES: CCOC(=O)c1c(-c2cc(C)c(C(F)(F)F)nc2N2C[C@H](C)C(F)(F)[C@@H](C)C2)[nH]c(C)c(C)c1=O]
ethyl 2-[2-[(3S,5S)-4,4-difluoro-3,5-dimethyl-1-
piperidyl]-5-methyl-6-(trifluoromethyl)-3-
pyridyl]-5,6-dimethyl-4-oxo-1H-pyridine-3-
carboxylate 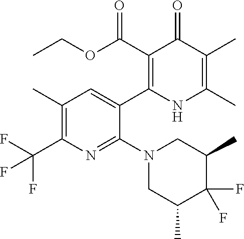 [SMILES: CCOC(=O)c1c(-c2cc(C)c(C(F)(F)F)nc2N2C[C@@H](C)C(F)(F)[C@H](C)C2)[nH]c(C)c(C)c1=O]
ethyl 2-[2-[(3R,5R)-4,4-difluoro-3,5-dimethyl-1-
piperidyl]-5-methyl-6-(trifluoromethyl)-3-
pyridyl]-5,6-dimethyl-4-oxo-1H-pyridine-3-
carboxylate 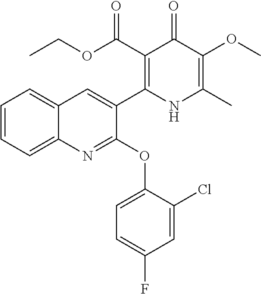 [SMILES: CCOC(=O)c1c(-c2cc3ccccc3nc2Oc2ccc(F)cc2Cl)[nH]c(C)c(OC)c1=O]
ethyl 2-[2-(2-chloro-4-fluoro-phenoxy)-3-
quinolyl]-5-methoxy-6-methyl-4-oxo-1H-
pyridine-3-carboxylate 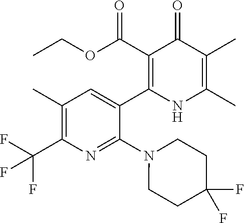 [SMILES: CCOC(=O)c1c(-c2cc(C)c(C(F)(F)F)nc2N2CCC(F)(F)CC2)[nH]c(C)c(C)c1=O]
ethyl 2-[2-(4,4-difluoro-1-piperidyl)-5-methyl-6-
(trifluoromethyl)-3-pyridyl]-5,6-dimethyl-4-oxo-
1H-pyridine-3-carboxylate 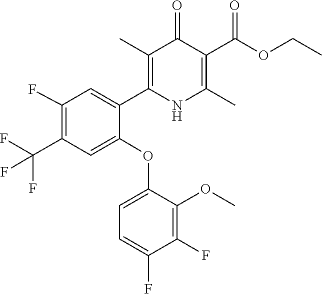 [SMILES: CCOC(=O)c1c(C)[nH]c(-c2cc(F)c(C(F)(F)F)cc2Oc2ccc(F)c(F)c2OC)c(C)c1=O]
ethyl 6-[2-(3,4-difluoro-2-methoxy-phenoxy)-5-
fluoro-4-(trifluoromethyl)phenyl]-2,5-dimethyl-4-
oxo-1H-pyridine-3-carboxylate 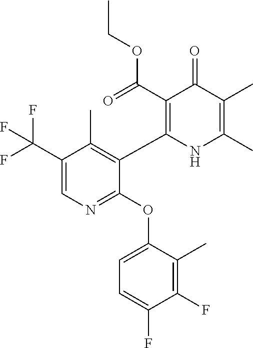 [SMILES: CCOC(=O)c1c(-c2c(Oc3ccc(F)c(F)c3C)ncc(C(F)(F)F)c2C)[nH]c(C)c(C)c1=O]
ethyl 2-[2-(3,4-difluoro-2-methyl-phenoxy)-4-
methyl-5-(trifluoromethyl)-3-pyridyl]-5,6-
dimethyl-4-oxo-1H-pyridine-3-carboxylate 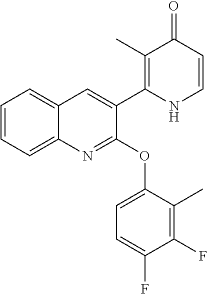 [SMILES: Cc1c(Oc2nc3ccccc3cc2-c2[nH]ccc(=O)c2C)ccc(F)c1F]
2-[2-(3,4-difluoro-2-methyl-phenoxy)-3-
quinolyl]-3-methyl-1H-pyridin-4-one 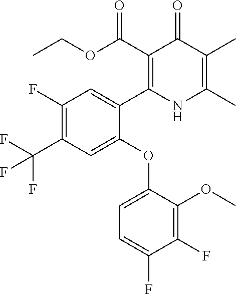 [SMILES: CCOC(=O)c1c(-c2cc(F)c(C(F)(F)F)cc2Oc2ccc(F)c(F)c2OC)[nH]c(C)c(C)c1=O]
ethyl 2-[2-(3,4-difluoro-2-methoxy-phenoxy)-5-
fluoro-4-(trifluoromethyl)phenyl]-5,6-dimethyl-4-
oxo-1H-pyridine-3-carboxylate 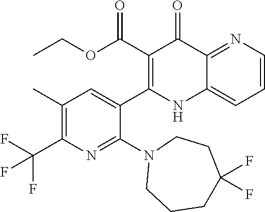 [SMILES: CCOC(=O)c1c(-c2cc(C)c(C(F)(F)F)nc2N2CCCC(F)(F)CC2)[nH]c2cccnc2c1=O]
2-[2-(4,4-difluoroazepan-1-yl)-5-methyl-6-
(trifluoromethyl)-3-pyridyl]-3-(2-
methoxyethoxy)-6-methyl-1H-pyridin-4-one 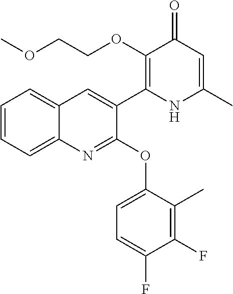 [SMILES: COCCOc1c(-c2cc3ccccc3nc2Oc2ccc(F)c(F)c2C)[nH]c(C)cc1=O]
2-[2-(3,4-difluoro-2-methyl-phenoxy)-3-
quinolyl]-3-(2-methoxyethoxy)-6-methyl-1H-
pyridin-4-one 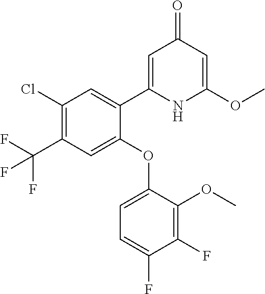 [SMILES: COc1cc(=O)cc(-c2cc(Cl)c(C(F)(F)F)cc2Oc2ccc(F)c(F)c2OC)[nH]1]
2-[5-chloro-2-(3,4-difluoro-2-methoxy-phenoxy)-
4-(trifluoromethyl)phenyl]-6-methoxy-1H-
pyridin-4-one 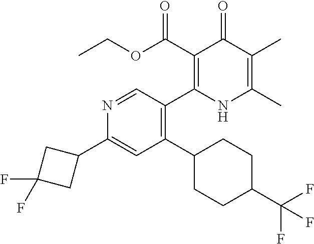 [SMILES: CCOC(=O)c1c(-c2cnc(C3CC(F)(F)C3)cc2C2CCC(C(F)(F)F)CC2)[nH]c(C)c(C)c1=O]
ethyl 2-[6-(3,3-difluorocyclobutyl)-4-[4-
(trifluoromethyl)cyclohexyl]-3-pyridyl]-5,6-
dimethyl-4-oxo-1H-pyridine-3-carboxylate 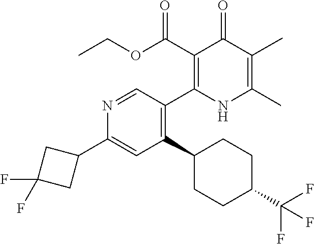 [SMILES: CCOC(=O)c1c(-c2cnc(C3CC(F)(F)C3)cc2[C@H]2CC[C@H](C(F)(F)F)CC2)[nH]c(C)c(C)c1=O]
ethyl 2-[6-(3,3-difluorocyclobutyl)-4-((1r,4r)-4-
(trifluoromethyl)cyclohexyl)-3-pyridyl]-5,6-
dimethyl-4-oxo-1H-pyridine-3-carboxylate 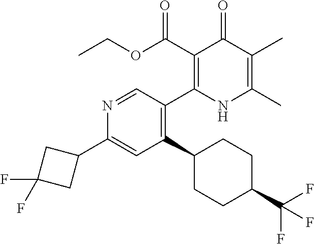 [SMILES: CCOC(=O)c1c(-c2cnc(C3CC(F)(F)C3)cc2[C@H]2CC[C@@H](C(F)(F)F)CC2)[nH]c(C)c(C)c1=O]
ethyl 2-[6-(3,3-difluorocyclobutyl)-4-((1s,4s)-4-
(trifluoromethyl)cyclohexyl)-3-pyridyl]-5,6-
dimethyl-4-oxo-1H-pyridine-3-carboxylate 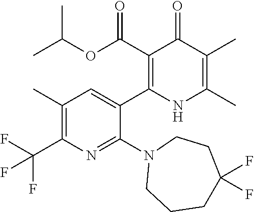 [SMILES: Cc1cc(-c2[nH]c(C)c(C)c(=O)c2C(=O)OC(C)C)c(N2CCCC(F)(F)CC2)nc1C(F)(F)F]
isopropyl 2-[2-(4,4-difluoroazepan-1-yl)-5-
methyl-6-(trifluoromethyl)-3-pyridyl]-5,6-
dimethyl-4-oxo-1H-pyridine-3-carboxylate 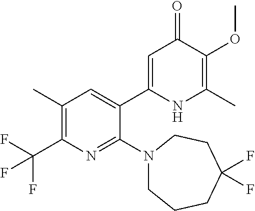 [SMILES: COc1c(C)[nH]c(-c2cc(C)c(C(F)(F)F)nc2N2CCCC(F)(F)CC2)cc1=O]
6-[2-(4,4-difluoroazepan-1-yl)-5-methyl-6-
(trifluoromethyl)-3-pyridyl]-3-methoxy-2-methyl-
1H-pyridin-4-one 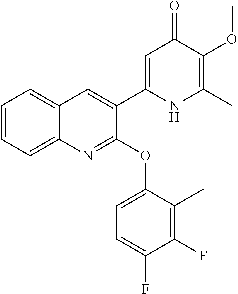 [SMILES: COc1c(C)[nH]c(-c2cc3ccccc3nc2Oc2ccc(F)c(F)c2C)cc1=O]
6-[2-(3,4-difluoro-2-methyl-phenoxy)-3-
quinolyl]-3-methoxy-2-methyl-1H-pyridin-4-one 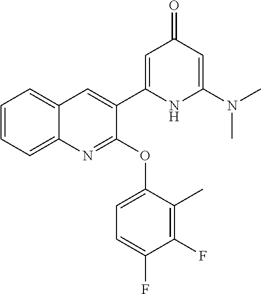 [SMILES: Cc1c(Oc2nc3ccccc3cc2-c2cc(=O)cc(N(C)C)[nH]2)ccc(F)c1F]
2-[2-(3,4-difluoro-2-methyl-phenoxy)-3-
quinolyl]-6-(dimethylamino)-1H-pyridin-4-one 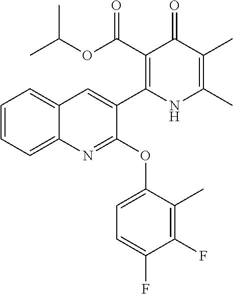 [SMILES: Cc1[nH]c(-c2cc3ccccc3nc2Oc2ccc(F)c(F)c2C)c(C(=O)OC(C)C)c(=O)c1C]
isopropyl 2-[2-(3,4-difluoro-2-methyl-phenoxy)-
3-quinolyl]-5,6-(dimethyl-4-oxo-1H-pyridine-3-
carboxylate 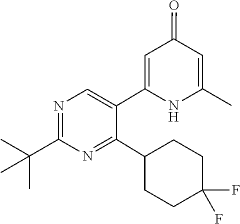 [SMILES: Cc1cc(=O)cc(-c2cnc(C(C)(C)C)nc2C2CCC(F)(F)CC2)[nH]1]
2-[2-tert-butyl-4-(4,4-
difluorocyclohexyl)pyrimidin-5-yl]-6-methyl-1H-
pyridin-4-one 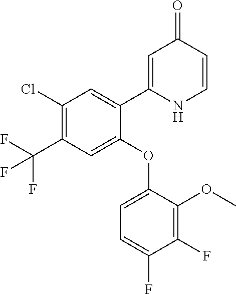 [SMILES: COc1c(Oc2cc(C(F)(F)F)c(Cl)cc2-c2cc(=O)cc[nH]2)ccc(F)c1F]
2-[5-chloro-2-(3,4-difluoro-2-methoxy-phenoxy)-
4-(trifluoromethyl)phenyl]-1H-pyridin-4-one 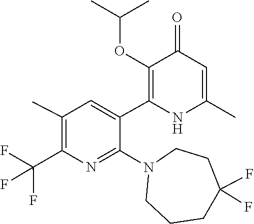 [SMILES: Cc1cc(=O)c(OC(C)C)c(-c2cc(C)c(C(F)(F)F)nc2N2CCCC(F)(F)CC2)[nH]1]
2-[2-(4,4-difluoroazepan-1-yl)-5-methyl-6-
(trifluoromethyl)-3-pyridyl]-3-isopropoxy-6-
methyl-1H-pyridin-4-one 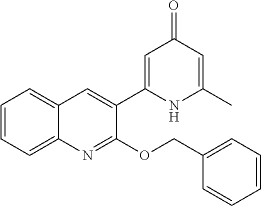 [SMILES: Cc1cc(=O)cc(-c2cc3ccccc3nc2OCc2ccccc2)[nH]1]
2-(2-benzyloxy-3-quinolyl)-6-methyl-1H-pyridin-
4-one 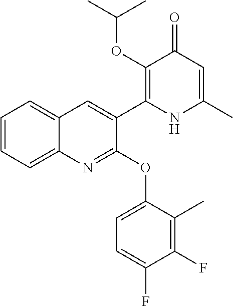 [SMILES: Cc1cc(=O)c(OC(C)C)c(-c2cc3ccccc3nc2Oc2ccc(F)c(F)c2C)[nH]1]
2-[2-(3,4-difluoro-2-methyl-phenoxy)-3-
quinolyl]-3-isopropoxy-6-methyl-1H-
pyridin-4-one 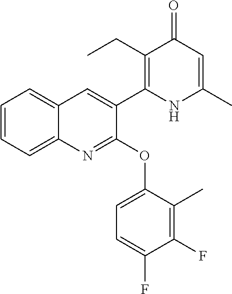 [SMILES: CCc1c(-c2cc3ccccc3nc2Oc2ccc(F)c(F)c2C)[nH]c(C)cc1=O]
2-[2-(3,4-difluoro-2-methyl-phenoxy)-3-
quinolyl]-3-methoxy-6-methyl-1H-
pyridin-4-one 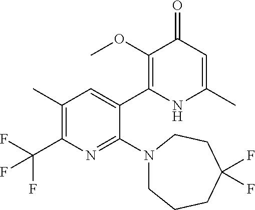 [SMILES: COc1c(-c2cc(C)c(C(F)(F)F)nc2N2CCCC(F)(F)CC2)[nH]c(C)cc1=O]
2-[2-(4,4-difluoroazepan-1-yl)-5-methyl-6-
(trifluoromethyl)-3-pyridyl]-3-isopropoxy-6-
methyl-1H-pyridin-4-one 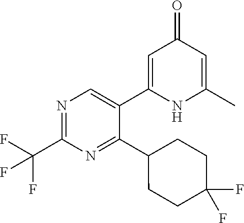 [SMILES: Cc1cc(=O)cc(-c2cnc(C(F)(F)F)nc2C2CCC(F)(F)CC2)[nH]1]
2-[4-(4,4-difluorocyclohexyl)-2-
(trifluoromethyl)pyrimidin-5-yl]-6-methyl-
1H-pyridin-4-one 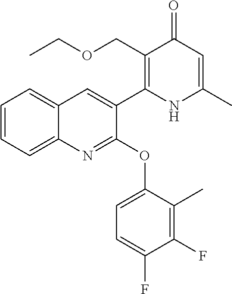 [SMILES: CCOCc1c(-c2cc3ccccc3nc2Oc2ccc(F)c(F)c2C)[nH]c(C)cc1=O]
2-[2-(3,4-difluoro-2-methyl-phenoxy)-3-
quinolyl]-3-(ethoxymethyl)-6-methyl-1H-
pyridin-4-one 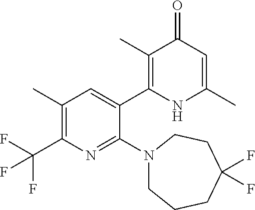 [SMILES: Cc1cc(=O)c(C)c(-c2cc(C)c(C(F)(F)F)nc2N2CCCC(F)(F)CC2)[nH]1]
2-[2-(4,4-difluoroazepan-1-yl)-5-methyl-6-
(trifluoromethyl)-3-pyridyl]-3,6-dimethyl-1H-
pyridin-4-one 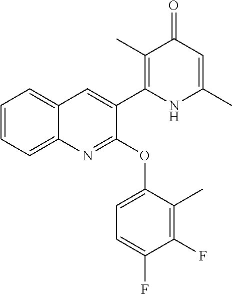 [SMILES: Cc1cc(=O)c(C)c(-c2cc3ccccc3nc2Oc2ccc(F)c(F)c2C)[nH]1]
2-[2-(3,4-difluoro-2-methyl-phenoxy)-3-
quinolyl]-3,6-dimethyl-1H-pyridin-4-one 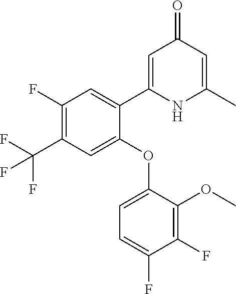 [SMILES: COc1c(Oc2cc(C(F)(F)F)c(F)cc2-c2cc(=O)cc(C)[nH]2)ccc(F)c1F]
2-[2-(3,4-difluoro-2-methoxy-phenoxy)-5-fluoro-
4-(trifluoromethyl)phenyl]-6-methyl-1H-
pyridin-4-one 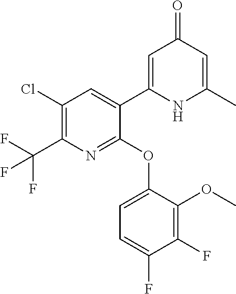 [SMILES: COc1c(Oc2nc(C(F)(F)F)c(Cl)cc2-c2cc(=O)cc(C)[nH]2)ccc(F)c1F]
2-[5-chloro-2-(3,4-difluoro-2-methoxy-phenoxy)-
4-(trifluoromethyl)phenyl]-6-methyl-1H-pyridin-
4-one 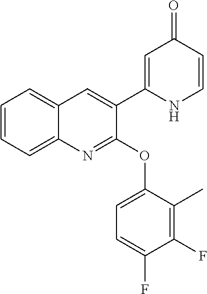 [SMILES: Cc1c(Oc2nc3ccccc3cc2-c2cc(=O)cc[nH]2)ccc(F)c1F]
2-[2-(3,4-difluoro-2-methyl-phenoxy)-3-
quinolyl]-1H-pyridin-4-one 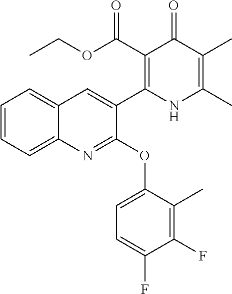 [SMILES: CCOC(=O)c1c(-c2cc3ccccc3nc2Oc2ccc(F)c(F)c2C)[nH]c(C)c(C)c1=O]
ethyl 2-[2-(3,4-difluoro-2-methyl-phenoxy)-3-
quinolyl]-5,6-dimethyl-4-oxo-1H-pyridine-
3-carboxylate 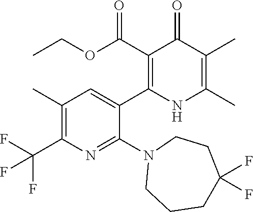 [SMILES: CCOC(=O)c1c(-c2cc(C)c(C(F)(F)F)nc2N2CCCC(F)(F)CC2)[nH]c(C)c(C)c1=O]
ethyl 2-[2-(4,4-difluoroazepan-1-yl)-5-methyl-6-
(trifluoromethyl)-3-pyridyl]-5,6-dimethyl-4-oxo-
1H-pyridine-3-carboxylate 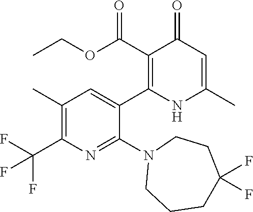 [SMILES: CCOC(=O)c1c(-c2cc(C)c(C(F)(F)F)nc2N2CCCC(F)(F)CC2)[nH]c(C)cc1=O]
ethyl 2-[2-(4,4-difluoroazepan-1-yl)-5-methyl-6-
(trifluoromethyl)-3-pyridyl]-6-methyl-4-oxo-
1H-pyridine-3-carboxylate 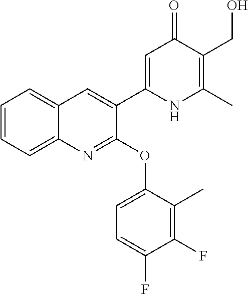 [SMILES: Cc1[nH]c(-c2cc3ccccc3nc2Oc2ccc(F)c(F)c2C)cc(=O)c1CO]
6-[2-(3,4-difluoro-2-methyl-phenoxy)-3-
quinolyl]-3-(hydroxymethyl)-2-methyl-1H-
pyridin-4-one 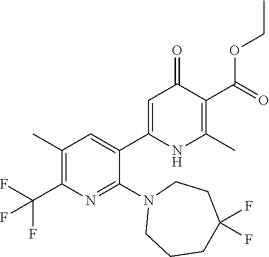 [SMILES: CCOC(=O)c1c(C)[nH]c(-c2cc(C)c(C(F)(F)F)nc2N2CCCC(F)(F)CC2)cc1=O]
ethyl 6-[2-(4,4-difluoroazepan-1-yl)-5-methyl-6-
(trifluoromethyl)-3-pyridyl]-2-methyl-4-oxo-1H-
pyridine-3-carboxylate 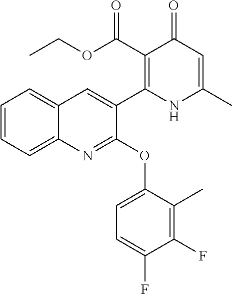 [SMILES: CCOC(=O)c1c(-c2cc3ccccc3nc2Oc2ccc(F)c(F)c2C)[nH]c(C)cc1=O]
ethyl 2-[2-(3,4-difluoro-2-methyl-phenoxy)-3-
quinolyl]-6-methyl-4-oxo-1H-pyridine-3-
carboxylate 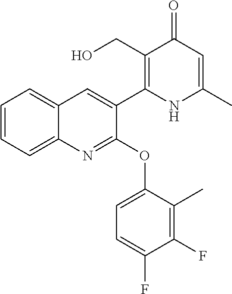 [SMILES: Cc1cc(=O)c(CO)c(-c2cc3ccccc3nc2Oc2ccc(F)c(F)c2C)[nH]1]
2-[2-(3,4-difluoro-2-methyl-phenoxy)-3-
quinolyl]-3-(hydroxymethyl)-6-methyl-1H-
pyridin-4-one 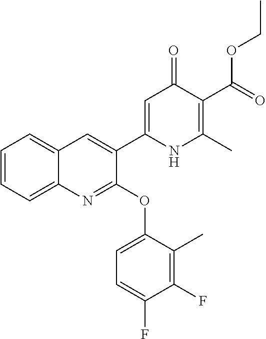 [SMILES: CCOC(=O)c1c(C)[nH]c(-c2cc3ccccc3nc2Oc2ccc(F)c(F)c2C)cc1=O]
ethyl 6-[2-(3,4-difluoro-2-methyl-phenoxy)-3-
quinolyl]-2-methyl-4-oxo-1H-pyridine-3-
carboxylate 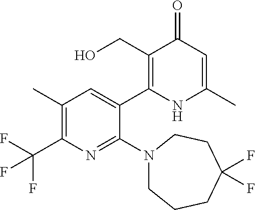 [SMILES: Cc1cc(=O)c(CO)c(-c2cc(C)c(C(F)(F)F)nc2N2CCCC(F)(F)CC2)[nH]1]
ethyl 6-[2-(4,4-difluoroazepan-1-yl)-5-methyl-6-
(trifluoromethyl)-3-pyridyl]-3-(hydroxymethyl)-
6-methyl-1H-pyridin-4-one 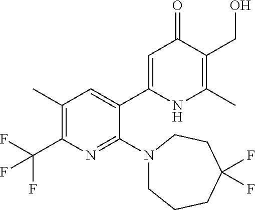 [SMILES: Cc1cc(-c2cc(=O)c(CO)c(C)[nH]2)c(N2CCCC(F)(F)CC2)nc1C(F)(F)F]
6-[2-(4,4-difluoroazepan-1-yl)-5-methyl-6-
(trifluoromethyl)-3-pyridyl]-3-(hydroxymethyl)-
2-methyl-1H-pyridin-4-one 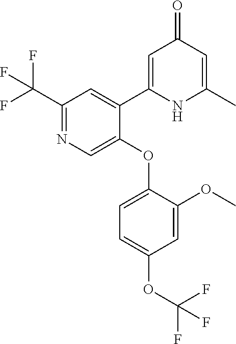 [SMILES: COc1cc(OC(F)(F)F)ccc1Oc1cnc(C(F)(F)F)cc1-c1cc(=O)cc(C)[nH]1]
2-[5-[2-methoxy-4-(trifluoromethyl)phenoxy]-
2-(trifluoromethyl)-4-pyridyl]-6-methyl-1H-
pyridin-4-one 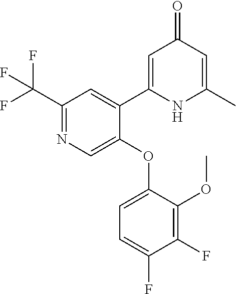 [SMILES: COc1c(Oc2cnc(C(F)(F)F)cc2-c2cc(=O)cc(C)[nH]2)ccc(F)c1F]
2-[5-(3,4-difluoro-2-methoxy-phenoxy)-2-
(trifluoromethyl)-4-pyridyl]-6-methyl-1H-
pyridin-4-one 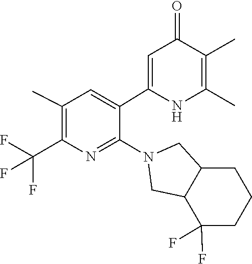 [SMILES: Cc1cc(-c2cc(=O)c(C)c(C)[nH]2)c(N2CC3CCCC(F)(F)C3C2)nc1C(F)(F)F]
6-[2-(7,7-difluoro-3,3a,4,5,6,7a-hexahydro-1H-
isoindol-2-yl)-5-methyl-6-(trifluoromethyl)-3-
pyridyl]-2,3-dimethyl-1H-pyridin-4-one 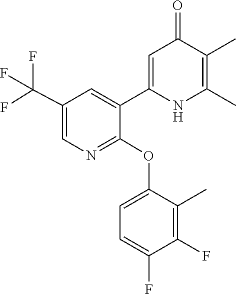 [SMILES: Cc1[nH]c(-c2cc(C(F)(F)F)cnc2Oc2ccc(F)c(F)c2C)cc(=O)c1C]
6-[2-(3,4-difluoro-2-methyl-phenoxy)-5-
(trifluoromethyl)-3-pyridyl]-2,3-dimethyl-1H-
pyridin-4-one 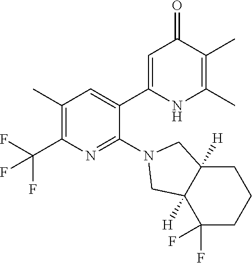 [SMILES: Cc1cc(-c2cc(=O)c(C)c(C)[nH]2)c(N2C[C@H]3CCCC(F)(F)[C@H]3C2)nc1C(F)(F)F]
6-[(3aR,7aS)-2-(7,7-difluoro-3,3a,4,5,6,7a-
hexahydro-1H-isoindol-2-yl)-5-methyl-6-
(trifluoromethyl)-3-pyridyl]-2,3-dimethyl-
1H-pyridin-4-one 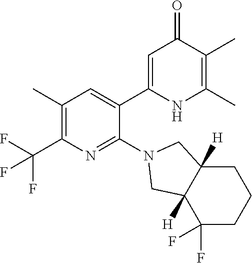 [SMILES: Cc1cc(-c2cc(=O)c(C)c(C)[nH]2)c(N2C[C@@H]3CCCC(F)(F)[C@@H]3C2)nc1C(F)(F)F]
6-[(3aS,7aR)-2-(7,7-difluoro-3,3a,4,5,6,7a-
hexahydro-1H-isoindol-2-yl)-5-methyl-6-
(trifluoromethyl)-3-pyridyl]-2,3-dimethyl-
1H-pyridin-4-one 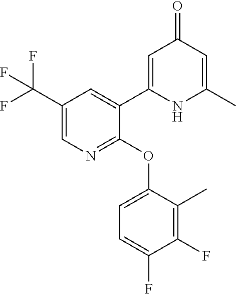 [SMILES: Cc1cc(=O)cc(-c2cc(C(F)(F)F)cnc2Oc2ccc(F)c(F)c2C)[nH]1]
2-[2-(3,4-difluoro-2-methyl-phenoxy)-5-
(trifluoromethyl)-3-pyridyl]-6-methyl-1H-
pyridin-4-one 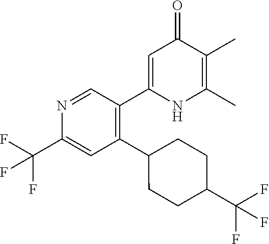 [SMILES: Cc1[nH]c(-c2cnc(C(F)(F)F)cc2C2CCC(C(F)(F)F)CC2)cc(=O)c1C]
2,3-dimethyl-6-[6-(trifluoromethyl)-4-[4-
(trifluoromethyl)cyclohexyl]-3-pyridyl]-1H-
pyridin-4-one 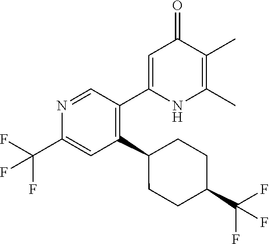 [SMILES: Cc1[nH]c(-c2cnc(C(F)(F)F)cc2[C@H]2CC[C@@H](C(F)(F)F)CC2)cc(=O)c1C]
2,3-dimethyl-6-[6-(trifluoromethyl)-4-((1s,4s)-4-
(trifluoromethyl)cyclohexyl)-3-pyridyl]-1H-
pyridin-4-one 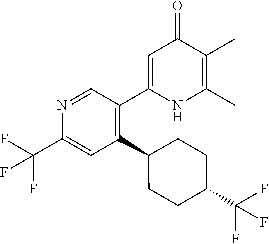 [SMILES: Cc1[nH]c(-c2cnc(C(F)(F)F)cc2[C@H]2CC[C@H](C(F)(F)F)CC2)cc(=O)c1C]
2,3-dimethyl-6-[6-(trifluoromethyl)-4-((1r,4r)-4-
(trifluoromethyl)cyclohexyl)-3-pyridyl]-1H-
pyridin-4-one 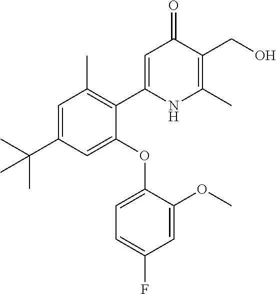 [SMILES: COc1cc(F)ccc1Oc1cc(C(C)(C)C)cc(C)c1-c1cc(=O)c(CO)c(C)[nH]1]
6-[4-tert-butyl-2-(4-fluoro-2-methoxy-phenoxy)-
6-methyl-phenyl]-3-(hydroxymethyl)-2-methyl-
1H-pyridin-4-one 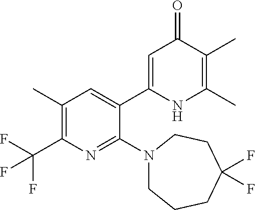 [SMILES: Cc1cc(-c2cc(=O)c(C)c(C)[nH]2)c(N2CCCC(F)(F)CC2)nc1C(F)(F)F]
6-[2-(4,4-difluoroazepan-1-yl)-5-methyl-6-
(trifluoromethyl)-3-pyridyl]-2,3-dimethyl-1H-
pyridin-4-one 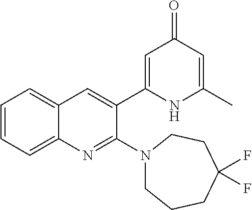 [SMILES: Cc1cc(=O)cc(-c2cc3ccccc3nc2N2CCCC(F)(F)CC2)[nH]1]
2-[2-(4,4-difluoroazepan-1-yl)-3-quinolyl]-6-
methyl-1H-pyridin-4-one 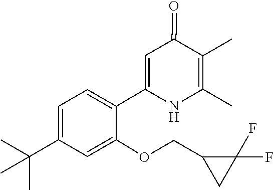 [SMILES: Cc1[nH]c(-c2ccc(C(C)(C)C)cc2OCC2CC2(F)F)cc(=O)c1C]
6-[4-tert-butyl-2-[(2,2-
difluorocyclopropyl)methoxy]phenyl]-2,3-
dimethyl-1H-pyridin-4-one 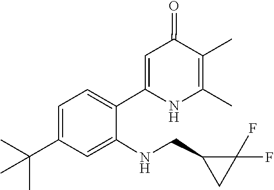 [SMILES: Cc1[nH]c(-c2ccc(C(C)(C)C)cc2NC[C@@H]2CC2(F)F)cc(=O)c1C]
(S)-6-[4-tert-butyl-2-[(2,2-
difluorocyclopropyl)methoxy]phenyl]-2,3-
dimethyl-1H-pyridin-4-one 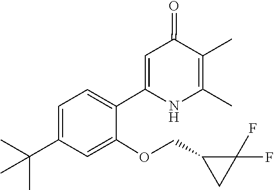 [SMILES: Cc1[nH]c(-c2ccc(C(C)(C)C)cc2OC[C@H]2CC2(F)F)cc(=O)c1C]
(R)-6-[4-tert-butyl-2-[(2,2-
difluorocyclopropyl)methoxy]phenyl]-2,3-
dimethyl-1H-pyridin-4-one 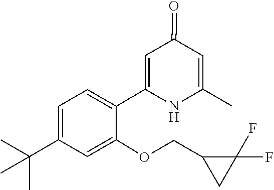 [SMILES: Cc1cc(=O)cc(-c2ccc(C(C)(C)C)cc2OCC2CC2(F)F)[nH]1]
2-[4-tert-butyl-2-[(2,2-
difluorocyclopropyl)methoxy]phenyl]-6-
methyl-1H-pyridin-4-one 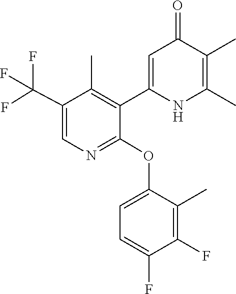 [SMILES: Cc1[nH]c(-c2c(Oc3ccc(F)c(F)c3C)ncc(C(F)(F)F)c2C)cc(=O)c1C]
6-[2-(3,4-difluoro-2-methyl-phenoxy)-4-methyl-
5-(trifluoromethyl)-3-pyridyl]-2,3-dimethyl-1H-
pyridin-4-one 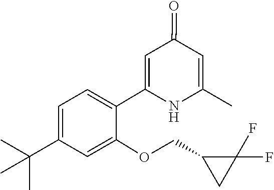 [SMILES: Cc1cc(=O)cc(-c2ccc(C(C)(C)C)cc2OC[C@H]2CC2(F)F)[nH]1]
(R)-2-[4-tert-butyl-2-[(2,2-
difluorocyclopropyl)methoxy]phenyl]-6-
methyl-1H-pyridin-4-one 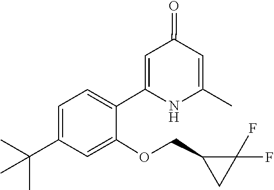 [SMILES: Cc1cc(=O)cc(-c2ccc(C(C)(C)C)cc2OC[C@@H]2CC2(F)F)[nH]1]
(S)-2-[4-tert-butyl-2-[(2,2-
difluorocyclopropyl)methoxy]phenyl]-6-
methyl-1H-pyridin-4-one 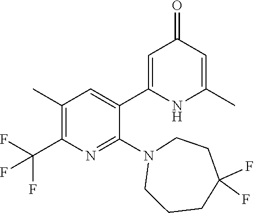 [SMILES: Cc1cc(=O)cc(-c2cc(C)c(C(F)(F)F)nc2N2CCCC(F)(F)CC2)[nH]1]
2-[2-(4,4-difluoroazepan-1-yl)-5-methyl-6-
(trifluoromethyl)-3-pyridyl]-6-methyl-1H-
pyridin-4-one 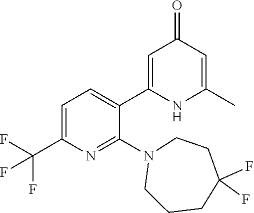 [SMILES: Cc1cc(=O)cc(-c2ccc(C(F)(F)F)nc2N2CCCC(F)(F)CC2)[nH]1]
2-[2-(4,4-difluoroazepan-1-yl)-6-
(trifluoromethyl)-3-pyridyl]-6-methyl-1H-
pyridin-4-one 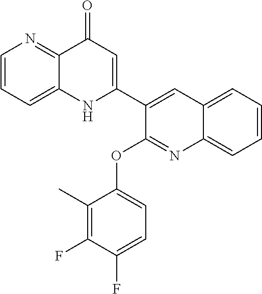 [SMILES: Cc1c(Oc2nc3ccccc3cc2-c2cc(=O)c3ncccc3[nH]2)ccc(F)c1F]
2-[2-(3,4-difluoro-2-methyl-phenoxy)-3-
quinolyl]-6-methyl-1H-pyridin-4-one 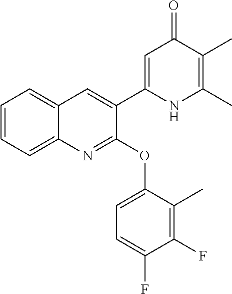 [SMILES: Cc1[nH]c(-c2cc3ccccc3nc2Oc2ccc(F)c(F)c2C)cc(=O)c1C]
6-[2-(3,4-difluoro-2-methyl-phenoxy)-3-
quinolyl]-2,3-dimethyl-1H-pyridin-4-one 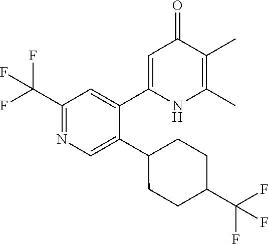 [SMILES: Cc1[nH]c(-c2cc(C(F)(F)F)ncc2C2CCC(C(F)(F)F)CC2)cc(=O)c1C]
2,3-dimethyl-6-[2-(trifluoromethyl)-5-[4-
(trifluoromethyl)cyclohexyl]-4-pyridyl]-1H-
pyridin-4-one 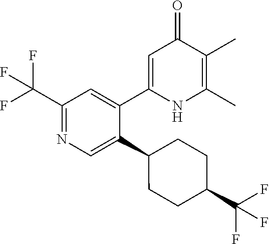 [SMILES: Cc1[nH]c(-c2cc(C(F)(F)F)ncc2[C@H]2CC[C@@H](C(F)(F)F)CC2)cc(=O)c1C]
2,3-dimethyl-6-[2-(trifluoromethyl)-5-((1s,4s)-4-
(trifluoromethyl)cyclohexyl)-4-pyridyl]-1H-
pyridin-4-one 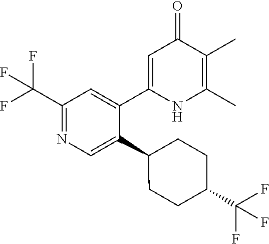 [SMILES: Cc1[nH]c(-c2cc(C(F)(F)F)ncc2[C@H]2CC[C@H](C(F)(F)F)CC2)cc(=O)c1C]
2,3-dimethyl-6-[2-(trifluoromethyl)-5-((1r,4r)-4-
(trifluoromethyl)cyclohexyl)-4-pyridyl]-1H-
pyridin-4-one 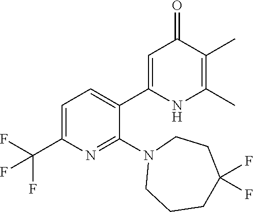 [SMILES: Cc1[nH]c(-c2ccc(C(F)(F)F)nc2N2CCCC(F)(F)CC2)cc(=O)c1C]
6-[2-(4,4-difluoroazepan-1-yl)-6-
(trifluoromethyl)-3-pyridyl]-2,3-dimethyl-1H-
pyridin-4-one 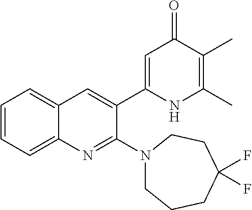 [SMILES: Cc1[nH]c(-c2cc3ccccc3nc2N2CCCC(F)(F)CC2)cc(=O)c1C]
6-[2-(4,4-difluoroazepan-1-yl)-3-quinolyl-2,3-
dimethyl-1H-pyridin-4-one 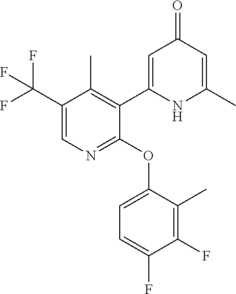 [SMILES: Cc1cc(=O)cc(-c2c(Oc3ccc(F)c(F)c3C)ncc(C(F)(F)F)c2C)[nH]1]
2-[2-(3,4-difluoro-2-methyl-phenoxy)-4-methyl-
5-(trifluoromethyl)-3-pyridyl]-6-methyl-1H-
pyridin-4-one 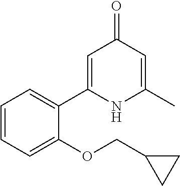 [SMILES: Cc1cc(=O)cc(-c2ccccc2OCC2CC2)[nH]1]
2-[2-(cyclopropylmethoxy)phenyl]-6-methyl-
1H-pyridin-4-one 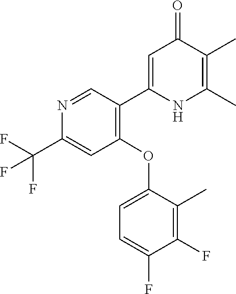 [SMILES: Cc1[nH]c(-c2cnc(C(F)(F)F)cc2Oc2ccc(F)c(F)c2C)cc(=O)c1C]
6-[4-(3,4-difluoro-2-methyl-phenoxy)-6-
(trifluoromethyl)-3-pyridyl]-2,3-dimethyl-1H-
pyridin-4-one 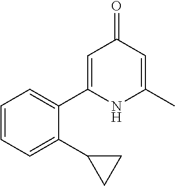 [SMILES: Cc1cc(=O)cc(-c2ccccc2C2CC2)[nH]1]
2-(2-cyclopropylphenyl)-6-methyl-1H-
pyridin-4-one 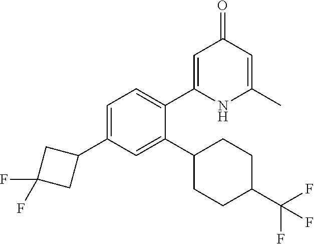 [SMILES: Cc1cc(=O)cc(-c2ccc(C3CC(F)(F)C3)cc2C2CCC(C(F)(F)F)CC2)[nH]1]
2-[4-(3,3-difluorocyclobutyl)-2-[4-
(trifluoromethyl)cyclohexyl]phenyl]-6-methyl-
1H-pyridin-4-one 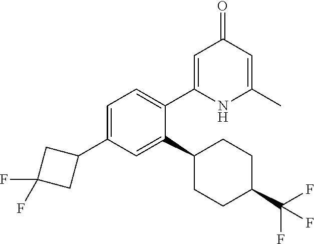 [SMILES: Cc1cc(=O)cc(-c2ccc(C3CC(F)(F)C3)cc2[C@H]2CC[C@@H](C(F)(F)F)CC2)[nH]1]
2-[4-(3,3-difluorocyclobutyl)-2-((1s,4s)-4-
(trifluoromethyl)cyclohexyl)phenyl]-6-methyl-
1H-pyridin-4-one 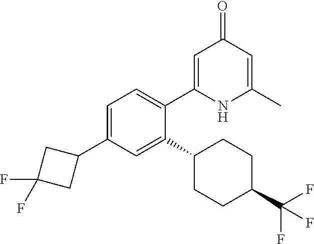 [SMILES: Cc1cc(=O)cc(-c2ccc(C3CC(F)(F)C3)cc2[C@H]2CC[C@H](C(F)(F)F)CC2)[nH]1]
2-[4-(3,3-difluorocyclobutyl)-2-((1r,4r)-4-
(trifluoromethyl)cyclohexyl)phenyl]-6-methyl-
1H-pyridin-4-one 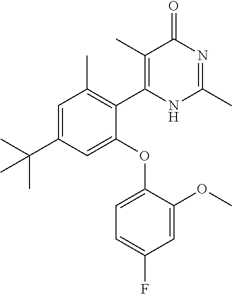 [SMILES: COc1cc(F)ccc1Oc1cc(C(C)(C)C)cc(C)c1-c1[nH]c(C)nc(=O)c1C]
6-[4-tert-butyl-2-(4-fluoro-2-methoxy-phenoxy)-
6-methyl-phenyl]-2,5-dimethyl-1H-pyridin-
4-one 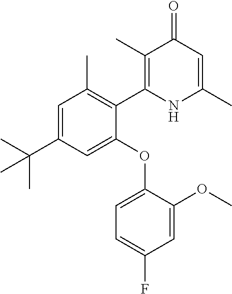 [SMILES: COc1cc(F)ccc1Oc1cc(C(C)(C)C)cc(C)c1-c1[nH]c(C)cc(=O)c1C]
2-[4-tert-butyl-2-(4-fluoro-2-methoxy-phenoxy)-
6-methyl-phenyl]-3,6-dimethyl-1H-pyridin-
4-one 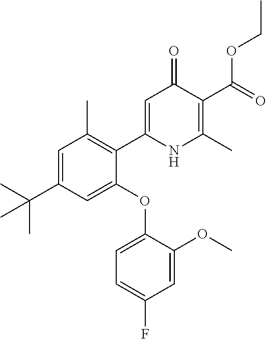 [SMILES: CCOC(=O)c1c(C)[nH]c(-c2c(C)cc(C(C)(C)C)cc2Oc2ccc(F)cc2OC)cc1=O]
ethyl 6-[4-tert-butyl-2-(4-fluoro-2-methoxy-
phenoxy)-6-methyl-phenyl]-2-methyl-4-oxo-
1H-pyridine-3-carboxylate 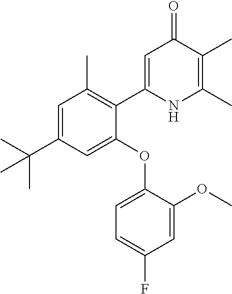 [SMILES: COc1cc(F)ccc1Oc1cc(C(C)(C)C)cc(C)c1-c1cc(=O)c(C)c(C)[nH]1]
6-[4-tert-butyl-2-(4-fluoro-2-methoxy-phenoxy)-
6-methyl-phenyl]-2,3-dimethyl-1H-pyridin-
4-one 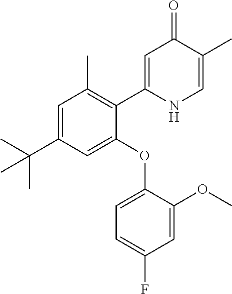 [SMILES: COc1cc(F)ccc1Oc1cc(C(C)(C)C)cc(C)c1-c1cc(=O)c(C)c[nH]1]
2-[4-tert-butyl-2-(4-fluoro-2-methoxy-phenoxy)-
6-methyl-phenyl]-5-methyl-1H-pyridin-
4-one 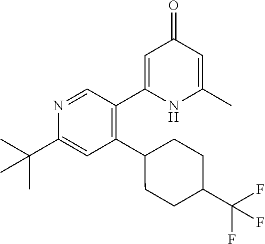 [SMILES: Cc1cc(=O)cc(-c2cnc(C(C)(C)C)cc2C2CCC(C(F)(F)F)CC2)[nH]1]
2-[6-tert-butyl-4-[4-(trifluoromethyl)cyclohexyl]-
3-pyridyl]-6-methyl-1H-pyridin-4-one 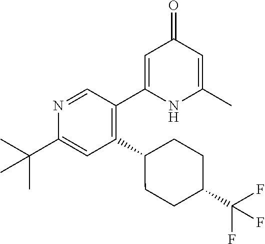 [SMILES: Cc1cc(=O)cc(-c2cnc(C(C)(C)C)cc2[C@H]2CC[C@@H](C(F)(F)F)CC2)[nH]1]
2-[6-tert-butyl-4-((1s,4s)-4-
(trifluoromethyl)cyclohexyl)-3-pyridyl]-
6-methyl-1H-pyridin-4-one 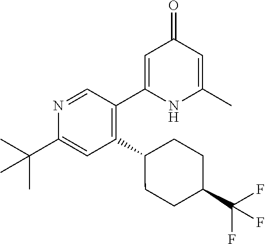 [SMILES: Cc1cc(=O)cc(-c2cnc(C(C)(C)C)cc2[C@H]2CC[C@H](C(F)(F)F)CC2)[nH]1]
2-[6-tert-butyl-4-((1r,4r)-4-
(trifluoromethyl)cyclohexyl]-3-pyridyl]-
6-methyl-1H-pyridin-4-one 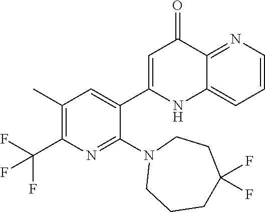 [SMILES: Cc1cc(-c2cc(=O)c3ncccc3[nH]2)c(N2CCCC(F)(F)CC2)nc1C(F)(F)F]
2-[2-(4,4-difluoroazepan-1-yl)-5-
(trifluoromethyl)-3-pyridyl]-6-methyl-
1H-pyridin-4-one 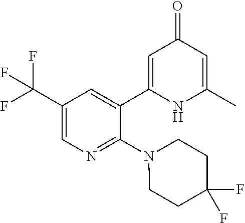 [SMILES: Cc1cc(=O)cc(-c2cc(C(F)(F)F)cnc2N2CCC(F)(F)CC2)[nH]1]
2-[2-(4,4-difluoro-1-piperidyl)-5-
(trifluoromethyl)-3-pyridyl]-6-methyl-
1H-pyridin-4-one 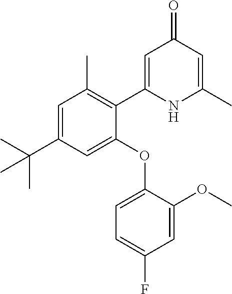 [SMILES: COc1cc(F)ccc1Oc1cc(C(C)(C)C)cc(C)c1-c1cc(=O)cc(C)[nH]1]
2-[4-tert-butyl-2-(4-fluoro-2-methoxy-phenoxy)-
6-methyl-phenyl]-6-methyl-1H-pyridin-
4-one 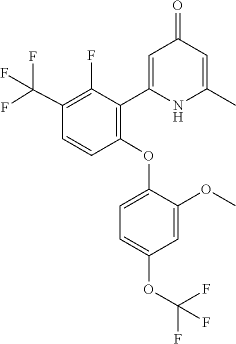 [SMILES: COc1cc(OC(F)(F)F)ccc1Oc1ccc(C(F)(F)F)c(F)c1-c1cc(=O)cc(C)[nH]1]
2-[2-fluoro-6-[2-methoxy-4-
(trifluoromethoxy)phenoxy]-3-
(trifluoromethyl)phenyl]-6-methyl-
1H-pyridin-4-one 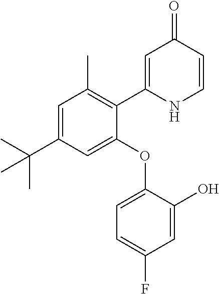 [SMILES: Cc1cc(C(C)(C)C)cc(Oc2ccc(F)cc2O)c1-c1cc(=O)cc[nH]1]
2-[4-tert-butyl-2-(4-fluoro-2-hydroxy-phenoxy)-
6-methyl-phenyl]-1H-pyridin-4-one 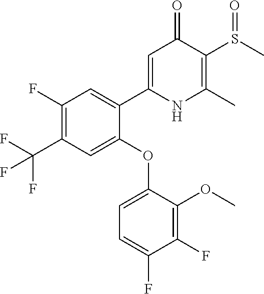 [SMILES: COc1c(Oc2cc(C(F)(F)F)c(F)cc2-c2cc(=O)c(S(C)=O)c(C)[nH]2)ccc(F)c1F]
6-[2-(3,4-difluoro-2-methoxy-phenoxy)-5-fluoro-
4-(trifluoromethyl)phenyl]-2-methyl-3-
methylsulfinyl-1H-pyridin-4-one 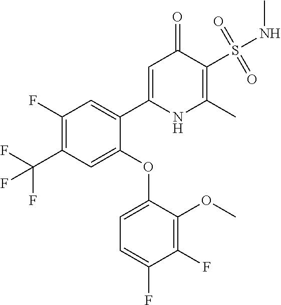 [SMILES: CNS(=O)(=O)c1c(C)[nH]c(-c2cc(F)c(C(F)(F)F)cc2Oc2ccc(F)c(F)c2OC)cc1=O]
6-[2-(3,4-difluoro-2-methoxy-phenoxy)-5-fluoro-
4-(trifluoromethyl)phenyl]-N,2-dimethyl-4-oxo-
1,4-dihydropyridine-3-sulfonamide 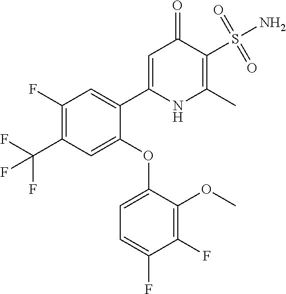 [SMILES: COc1c(Oc2cc(C(F)(F)F)c(F)cc2-c2cc(=O)c(S(N)(=O)=O)c(C)[nH]2)ccc(F)c1F]
6-[2-(3,4-difluoro-2-methoxy-phenoxy)-5-fluoro-
4-(trifluoromethyl)phenyl]-2-methyl-4-oxo-1H-
pyridine-3-sulfonamide 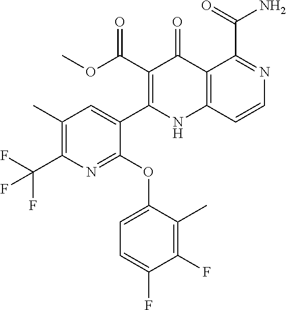 [SMILES: COC(=O)c1c(-c2cc(C)c(C(F)(F)F)nc2Oc2ccc(F)c(F)c2C)[nH]c2ccnc(C(N)=O)c2c1=O]
ethyl 2-[2-(3,4-difluoro-2-methoxy-phenoxy)-5-
fluoro-4-(trifluoromethyl)phenyl]-6-methyl-5-
(methylsulfonimidoyl)-4-oxo-1H-
pyridine-3-sulfonamide 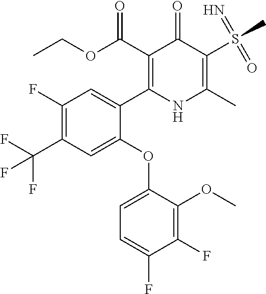 [SMILES: CCOC(=O)c1c(-c2cc(F)c(C(F)(F)F)cc2Oc2ccc(F)c(F)c2OC)[nH]c(C)c([S@](C)(=N)=O)c1=O]
ethyl (R)-2-[2-(3,4-difluoro-2-methoxy-phenoxy)-
5-fluoro-4-(trifluoromethyl)phenyl]-6-methyl-5-
(methylsulfonimidoyl)-4-oxo-1H-pyridine-
3-sulfonamide 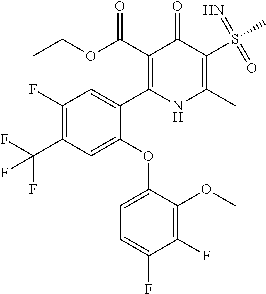 [SMILES: CCOC(=O)c1c(-c2cc(F)c(C(F)(F)F)cc2Oc2ccc(F)c(F)c2OC)[nH]c(C)c([S@@](C)(=N)=O)c1=O]
ethyl (S)-2-[2-(3,4-difluoro-2-methoxy-phenoxy)-
5-fluoro-4-(trifluoromethyl)phenyl]-6-methyl-5-
(methylsulfonimidoyl)-4-oxo-1H-pyridine-
3-sulfonamide 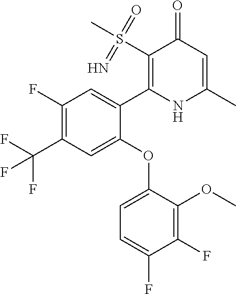 [SMILES: COc1c(Oc2cc(C(F)(F)F)c(F)cc2-c2[nH]c(C)cc(=O)c2S(C)(=N)=O)ccc(F)c1F]
2-[2-(3,4-difluoro-2-methoxy-phenoxy)-
5-fluoro-4-(trifluoromethyl)phenyl]-6-
methyl-3-(methylsulfonimidoyl)-1H-
pyridine-4-one 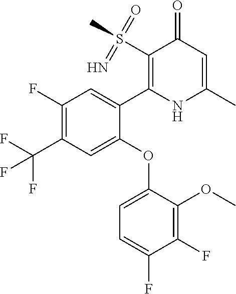 [SMILES: COc1c(Oc2cc(C(F)(F)F)c(F)cc2-c2[nH]c(C)cc(=O)c2[S@](C)(=N)=O)ccc(F)c1F]
(R)-2-[2-(3,4-difluoro-2-methoxy-phenoxy)-5-
fluoro-4-(trifluoromethyl)phenyl]-6-methyl-3-
(methylsulfonimidoyl)-1H-pyridine-4-one 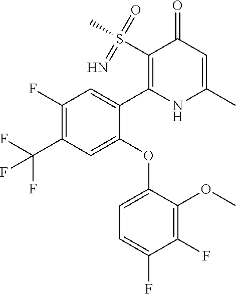 [SMILES: COc1c(Oc2cc(C(F)(F)F)c(F)cc2-c2[nH]c(C)cc(=O)c2[S@@](C)(=N)=O)ccc(F)c1F]
(S)-2-[2-(3,4-difluoro-2-methoxy-phenoxy)-5-
fluoro-4-(trifluoromethyl)phenyl]-6-methyl-3-
(methylsulfonimidoyl)-1H-pyridine-4-one 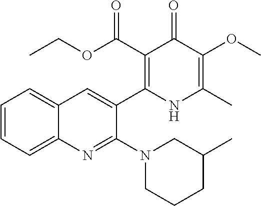 [SMILES: CCOC(=O)c1c(-c2cc3ccccc3nc2N2CCCC(C)C2)[nH]c(C)c(OC)c1=O]
ethyl 5-methoxy-6-methyl-2-[2-[3-methyl-1-
piperidyl]-3-quinolyl]-4-oxo-1H-pyridine-3-
carboxylate 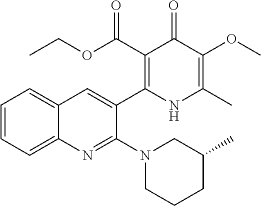 [SMILES: CCOC(=O)c1c(-c2cc3ccccc3nc2N2CCC[C@@H](C)C2)[nH]c(C)c(OC)c1=O]
ethyl 5-methoxy-6-methyl-2-[2-[(3S)-3-methyl-1-
piperidyl]-3-quinolyl]-4-oxo-1H-pyridine-3-
carboxylate 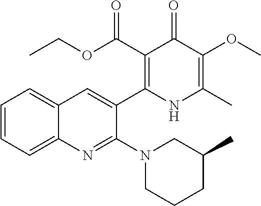 [SMILES: CCOC(=O)c1c(-c2cc3ccccc3nc2N2CCC[C@H](C)C2)[nH]c(C)c(OC)c1=O]
ethyl 5-methoxy-6-methyl-2-[2-[(3R)-3-methyl-1-
piperidyl]-3-quinolyl]-4-oxo-1H-pyridine-3-
carboxylate 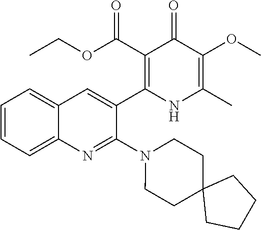 [SMILES: CCOC(=O)c1c(-c2cc3ccccc3nc2N2CCC3(CCCC3)CC2)[nH]c(C)c(OC)c1=O]
ethyl 2-[2-(8-azaspiro[4.5]decan-8-yl)-3-
quinolyl]-5-methoxy-6-methyl-4-oxo-1H-
pyridine-3-carboxylate 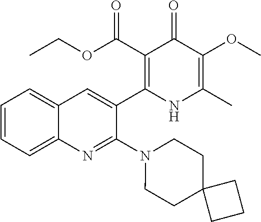 [SMILES: CCOC(=O)c1c(-c2cc3ccccc3nc2N2CCC3(CCC3)CC2)[nH]c(C)c(OC)c1=O]
ethyl 2-[2-(7-azaspiro[3.5]nonan-7-yl)-3-
quinolyl]-5-methoxy-6-methyl-4-oxo-1H-
pyridine-3-carboxylate 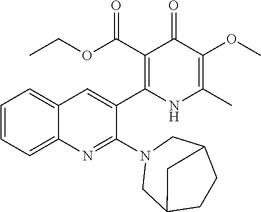 [SMILES: CCOC(=O)c1c(-c2cc3ccccc3nc2N2CC3CCC(C3)C2)[nH]c(C)c(OC)c1=O]
ethyl 2-[2-(3-azasbicyclo[3.2.1]octan-3-yl)-3-
quinolyl]-5-methoxy-6-methyl-4-oxo-1H-
pyridine-3-carboxylate 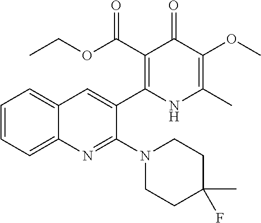 [SMILES: CCOC(=O)c1c(-c2cc3ccccc3nc2N2CCC(C)(F)CC2)[nH]c(C)c(OC)c1=O]
ethyl 2-[2-(4-fluoro-4-methyl-1-piperidyl)-3-
quinolyl]-5-methoxy-6-methyl-4-oxo-1H-
pyridine-3-carboxylate 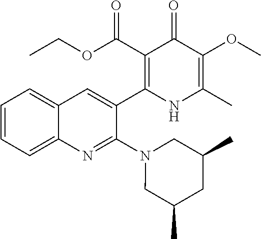 [SMILES: CCOC(=O)c1c(-c2cc3ccccc3nc2N2C[C@H](C)C[C@H](C)C2)[nH]c(C)c(OC)c1=O]
ethyl 2-[2-[(3S,5R)-3,5-dimethyl-1-piperidyl]-3-
quinolyl]-5-methoxy-6-methyl-4-oxo-1H-
pyridine-3-carboxylate 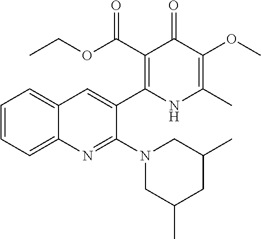 [SMILES: CCOC(=O)c1c(-c2cc3ccccc3nc2N2CC(C)CC(C)C2)[nH]c(C)c(OC)c1=O]
ethyl 2-[2-[3,5-dimethyl-1-piperidyl]-3-quinolyl]-
5-methoxy-6-methyl-4-oxo-1H-pyridine-3-
carboxylate 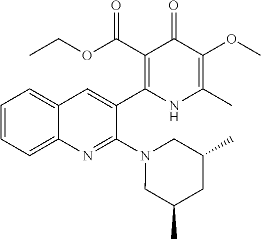 [SMILES: CCOC(=O)c1c(-c2cc3ccccc3nc2N2C[C@H](C)C[C@@H](C)C2)[nH]c(C)c(OC)c1=O]
ethyl 2-[2-[(3R,5R)-3,5-dimethyl-1-piperidyl]-3-
quinolyl]-5-methoxy-6-methyl-4-oxo-1H-
pyridine-3-carboxylate 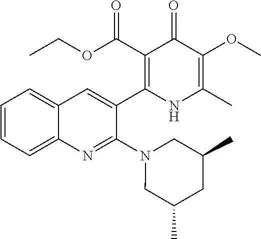 [SMILES: CCOC(=O)c1c(-c2cc3ccccc3nc2N2C[C@@H](C)C[C@H](C)C2)[nH]c(C)c(OC)c1=O]
ethyl 2-[2-[(3S,5S)-3,5-dimethyl-1-piperidyl]-3-
quinolyl]-5-methoxy-6-methyl-4-oxo-1H-
pyridine-3-carboxylate 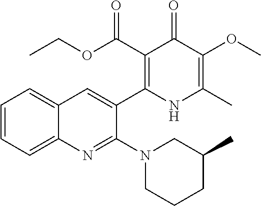 [SMILES: CCOC(=O)c1c(-c2cc3ccccc3nc2N2CCC[C@H](C)C2)[nH]c(C)c(OC)c1=O]
ethyl 5-methoxy-6-methyl-2-[2-[(3R)-3-methyl-1-
piperidyl]-3-quinolyl]-4-oxo-1H-pyridine-3-
carboxylate 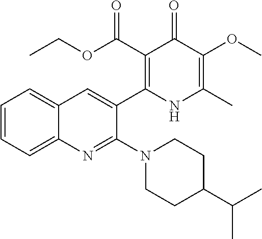 [SMILES: CCOC(=O)c1c(-c2cc3ccccc3nc2N2CCC(C(C)C)CC2)[nH]c(C)c(OC)c1=O]
ethyl 2-[2-(4-isopropyl-1-piperidyl)-3-quinolyl]-
5-methoxy-6-methyl-4-oxo-1H-pyridine-3-
carboxylate 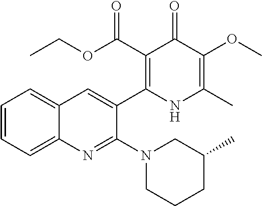 [SMILES: CCOC(=O)c1c(-c2cc3ccccc3nc2N2CCC[C@@H](C)C2)[nH]c(C)c(OC)c1=O]
ethyl 5-methoxy-6-methyl-2-[2-[(3S)-3-methyl-1-
piperidyl]-3-quinolyl]-4-oxo-1H-pyridine-3-
carboxylate 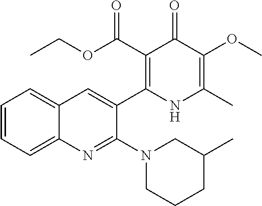 [SMILES: CCOC(=O)c1c(-c2cc3ccccc3nc2N2CCCC(C)C2)[nH]c(C)c(OC)c1=O]
ethyl 5-methoxy-6-methyl-2-[2-[3-methyl-1-
piperidyl]-3-quinolyl]-4-oxo-1H-pyridine-3-
carboxylate 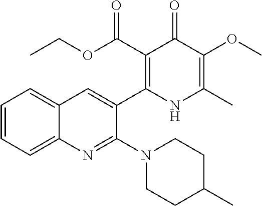 [SMILES: CCOC(=O)c1c(-c2cc3ccccc3nc2N2CCC(C)CC2)[nH]c(C)c(OC)c1=O]
ethyl 5-methoxy-6-methyl-2-[2-(4-methyl-1-
piperidyl]-3-quinolyl]-4-oxo-1H-pyridine-3-
carboxylate 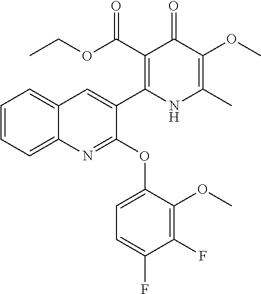 [SMILES: CCOC(=O)c1c(-c2cc3ccccc3nc2Oc2ccc(F)c(F)c2OC)[nH]c(C)c(OC)c1=O]
ethyl 2-[2-(3,4-difluoro-2-methoxy-phenoxy)-3-
quinolyl]-5-methoxy-6-methyl-4-oxo-1H-
pyridine-3-carboxylate 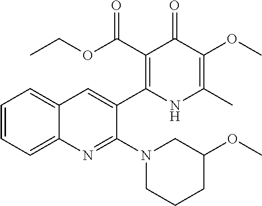 [SMILES: CCOC(=O)c1c(-c2cc3ccccc3nc2N2CCCC(OC)C2)[nH]c(C)c(OC)c1=O]
ethyl 5-methoxy-2-[2-(3-methoxy-1-piperidyl]-3-
quinolyl]-6-methyl-4-oxo-1H-pyridine-3-
carboxylate 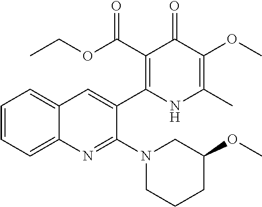 [SMILES: CCOC(=O)c1c(-c2cc3ccccc3nc2N2CCC[C@H](OC)C2)[nH]c(C)c(OC)c1=O]
ethyl 5-methoxy-2-[2-[(3R)-3-methoxy-1-
piperidyl]-3-quinolyl]-6-methyl-4-oxo-1H-
pyridine-3-carboxylate 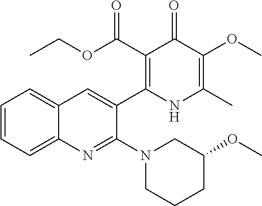 [SMILES: CCOC(=O)c1c(-c2cc3ccccc3nc2N2CCC[C@@H](OC)C2)[nH]c(C)c(OC)c1=O]
ethyl 5-methoxy-2-[2-[(3S)-3-methoxy-1-
piperidyl]-3-quinolyl]-6-methyl-4-oxo-1H-
pyridine-3-carboxylate 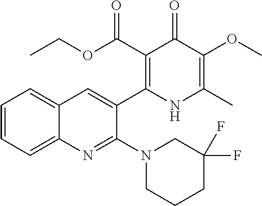 [SMILES: CCOC(=O)c1c(-c2cc3ccccc3nc2N2CCCC(F)(F)C2)[nH]c(C)c(OC)c1=O]
ethyl 2-[2-(3,3-difluoro-1-piperidyl)-3-quinolyl]-
5-methoxy-6-methyl-4-oxo-1H-pyridine-3-
carboxylate 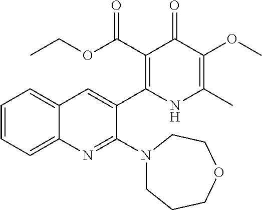 [SMILES: CCOC(=O)c1c(-c2cc3ccccc3nc2N2CCCOCC2)[nH]c(C)c(OC)c1=O]
ethyl 5-methoxy-6-methyl-2-[2-(1,4-oxazepan-4-
yl)-3-quinolyl]-4-oxo-1H-pyridine-3-carboxylate 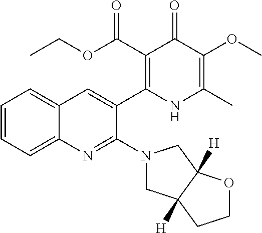 [SMILES: CCOC(=O)c1c(-c2cc3ccccc3nc2N2C[C@H]3CCO[C@H]3C2)[nH]c(C)c(OC)c1=O]
ethyl 2-[2-[(3aS,6aS)-2,3,3a,4,6,6a-
hexahydrofuro[2,3-c]pyrrol-5-yl]-3-quinolyl]-
5-methoxy-6-methyl-4-oxo-1H-pyridine-3-
carboxylate 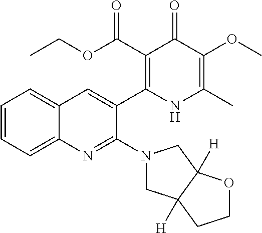 [SMILES: CCOC(=O)c1c(-c2cc3ccccc3nc2N2CC3CCOC3C2)[nH]c(C)c(OC)c1=O]
ethyl 2-[2-[2,3,3a,4,6,6a-hexahydrofuro[2,3-
c]pyrrol-5-yl]-3-quinolyl]-5-methoxy-6-methyl-
4-oxo-1H-pyridine-3-carboxylate 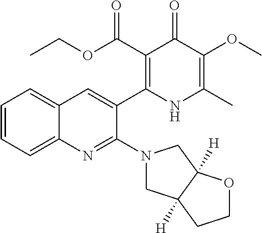 [SMILES: CCOC(=O)c1c(-c2cc3ccccc3nc2N2C[C@@H]3CCO[C@@H]3C2)[nH]c(C)c(OC)c1=O]
ethyl 2-[2-[(3aR,6aR)-2,3,3a,4,6,6a-
hexahydrofuro[2,3-c]pyrrol-5-yl]-3-quinolyl]-
5-methoxy-6-methyl-4-oxo-1H-pyridine-3-
carboxylate 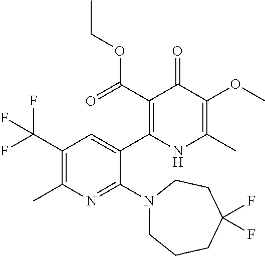 [SMILES: CCOC(=O)c1c(-c2cc(C(F)(F)F)c(C)nc2N2CCCC(F)(F)CC2)[nH]c(C)c(OC)c1=O]
ethyl 2-[2-(4,4-difluoroazepan-1-yl)-6-methyl-5-
(trifluoromethyl)-3-pyridyl]-5-methoxy-6-methyl-
4-oxo-1H-pyridine-3-carboxylate 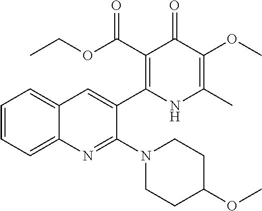 [SMILES: CCOC(=O)c1c(-c2cc3ccccc3nc2N2CCC(OC)CC2)[nH]c(C)c(OC)c1=O]
ethyl 5-methoxy-2-[2-(4-methoxy-1-piperidyl)-3-
quinolyl]-6-methyl-oxo-1H-pyridine-3-
carboxylate 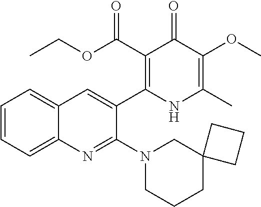 [SMILES: CCOC(=O)c1c(-c2cc3ccccc3nc2N2CCCC3(CCC3)C2)[nH]c(C)c(OC)c1=O]
ethyl 2-[2-(6-azaspiro[3.5]nonan-6-yl)-3-
quinolyl]-5-methoxy-6-methyl-4-oxo-1H-
pyridine-3-carboxylate 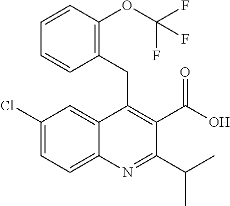 [SMILES: CC(C)c1nc2ccc(Cl)cc2c(Cc2ccccc2OC(F)(F)F)c1C(=O)O]
ethyl 5-methoxy-6-methyl-4-oxo-2-[2-[4-
(trifluoromethyl)-1-piperidyl]-3-quinolyl]-1H-
pyridine-3-carboxylate 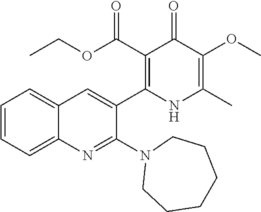 [SMILES: CCOC(=O)c1c(-c2cc3ccccc3nc2N2CCCCCC2)[nH]c(C)c(OC)c1=O]
ethyl 2-[2-(azepan-1-yl)-3-quinolyl]-5-methoxy-
6-methyl-4-oxo-1H-pyridine-3-carboxylate 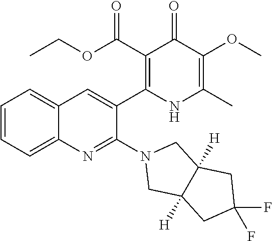 [SMILES: CCOC(=O)c1c(-c2cc3ccccc3nc2N2C[C@@H]3CC(F)(F)C[C@@H]3C2)[nH]c(C)c(OC)c1=O]
ethyl 2-[2-[(3aS,6aR)-5,5-difluoro-1,3,3a,4,6,6a-
hexahydrocyclopenta[c]pyrrol-2-yl]-3-quinolyl]-
5-methoxy-6-methyl-4-oxo-1H-pyridine-3-
carboxylate 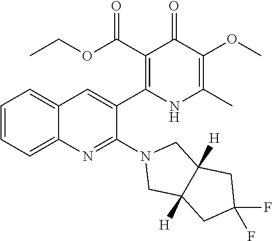 [SMILES: CCOC(=O)c1c(-c2cc3ccccc3nc2N2C[C@@H]3CC(F)(F)C[C@@H]3C2)[nH]c(C)c(OC)c1=O]
ethyl 2-[2-[(3aR,6aS)-5,5-difluoro-1,3,3a,4,6,6a-
hexahydrocyclopenta[c]pyrrol-2-yl]-3-quinolyl]-
5-methoxy-6-methyl-4-oxo-1H-pyridine-3-
carboxylate 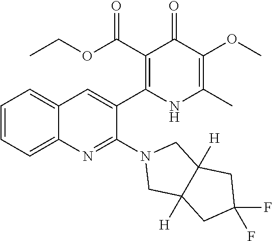 [SMILES: CCOC(=O)c1c(-c2cc3ccccc3nc2N2CC3CC(F)(F)CC3C2)[nH]c(C)c(OC)c1=O]
ethyl 2-[2-5,5-difluoro-1,3,3a,4,6,6a-
hexahydrocyclopenta[c]pyrrol-2-yl]-3-quinolyl]-
5-methoxy-6-methyl-4-oxo-1H-pyridine-3-
carboxylate 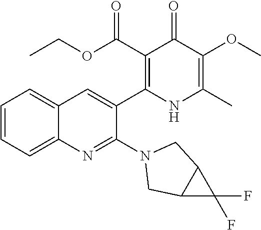 [SMILES: CCOC(=O)c1c(-c2cc3ccccc3nc2N2CC3C(C2)C3(F)F)[nH]c(C)c(OC)c1=O]
ethyl 2-[2-(6,6-difluoro-3-azabicyclo[3.1.0]hexan-
3-yl)-3-quinolyl]-5-methoxy-6-methyl-4-oxo-
1H-pyridine-3-carboxylate 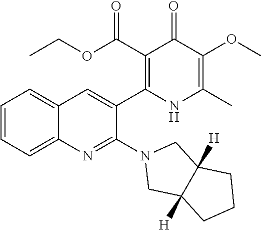 [SMILES: CCOC(=O)c1c(-c2cc3ccccc3nc2N2C[C@H]3CCC[C@H]3C2)[nH]c(C)c(OC)c1=O]
ethyl 2-[2-[(3aR,6aS)-3,3a,4,5,6,6a-hexahydro-
1H-cyclopenta[c]pyrrol-2-yl]-3-quinolyl]-5-
methoxy-6-methyl-4-oxo-1H-pyridine-3-
carboxylate 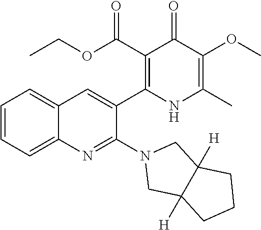 [SMILES: CCOC(=O)c1c(-c2cc3ccccc3nc2N2CC3CCCC3C2)[nH]c(C)c(OC)c1=O]
ethyl 2-[2-[3,3a,4,5,6,6a-hexahydro-1H-
cyclopenta[c]pyrrol-2-yl]-3-quinolyl]-5-
methoxy-6-methyl-4-oxo-1H-pyridine-3-
carboxylate 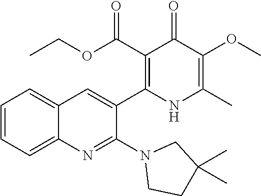 [SMILES: CCOC(=O)c1c(-c2cc3ccccc3nc2N2CCC(C)(C)C2)[nH]c(C)c(OC)c1=O]
ethyl 2-[2-(3,3-dimethylpyrrolidin-1-yl)-3-
quinolyl]-5-methoxy-6-methyl-4-oxo-1H-
pyridine-3-carboxylate 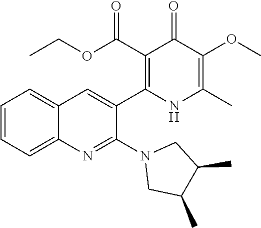 [SMILES: CCOC(=O)c1c(-c2cc3ccccc3nc2N2C[C@@H](C)[C@@H](C)C2)[nH]c(C)c(OC)c1=O]
ethyl 2-[2-[(3S,4R)-3,4-dimethylpyrrolidin-1-yl)-
3-quinolyl]-5-methoxy-6-methyl-4-oxo-1H-
pyridine-3-carboxylate 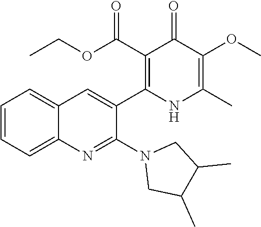 [SMILES: CCOC(=O)c1c(-c2cc3ccccc3nc2N2CC(C)C(C)C2)[nH]c(C)c(OC)c1=O]
ethyl 2-[2-[3,4-dimethylpyrrolidin-1-yl]-3-
quinolyl]-5-methoxy-6-methyl-4-oxo-1H-
pyridine-3-carboxylate 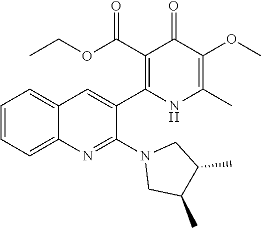 [SMILES: CCOC(=O)c1c(-c2cc3ccccc3nc2N2C[C@H](C)[C@@H](C)C2)[nH]c(C)c(OC)c1=O]
ethyl 2-[2-[(3R,4R)-3,4-dimethylpyrrolidin-1-yl)-
3-quinolyl]-5-methoxy-6-methyl-4-oxo-1H-
pyridine-3-carboxylate 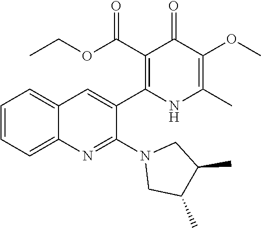 [SMILES: CCOC(=O)c1c(-c2cc3ccccc3nc2N2C[C@@H](C)[C@H](C)C2)[nH]c(C)c(OC)c1=O]
ethyl 2-[2-[(3S,4S)-3,4-dimethylpyrrolidin-1-yl]-
3-quinolyl]-5-methoxy-6-methyl-4-oxo-1H-
pyridine-3-carboxylate 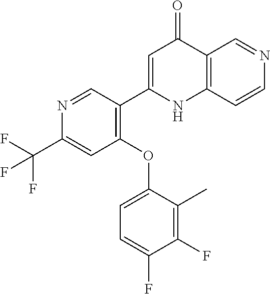 [SMILES: Cc1c(Oc2cc(C(F)(F)F)ncc2-c2cc(=O)c3cnccc3[nH]2)ccc(F)c1F]
2-[4-(3,4-difluoro-2-methyl-phenoxy)-6-
(trifluoromethyl)-3-pyridyl]-6-methyl-1H-
pyridin-4-one 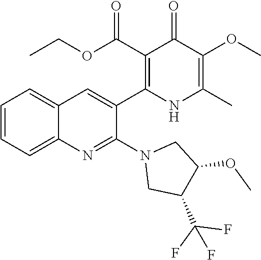 [SMILES: CCOC(=O)c1c(-c2cc3ccccc3nc2N2C[C@H](OC)[C@H](C(F)(F)F)C2)[nH]c(C)c(OC)c1=O]
ethyl 5-methoxy-2-[2-[(3S,4S)-3-methoxy-4-
(trifluoromethyl)pyrrolidin-1-yl]-3-quinolyl]-6-
methyl-4-oxo-1H-pyridine-3-carboxylate 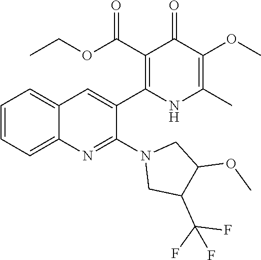 [SMILES: CCOC(=O)c1c(-c2cc3ccccc3nc2N2CC(OC)C(C(F)(F)F)C2)[nH]c(C)c(OC)c1=O]
ethyl 5-methoxy-2-[2-[3-methoxy-4-
(trifluoromethyl)pyrrolidin-1-yl]-3-quinolyl]-6-
methyl-4-oxo-1H-pyridine-3-carboxylate 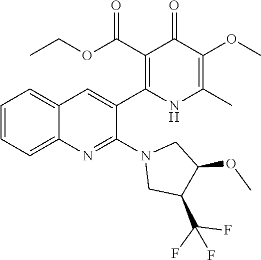 [SMILES: CCOC(=O)c1c(-c2cc3ccccc3nc2N2C[C@@H](OC)[C@@H](C(F)(F)F)C2)[nH]c(C)c(OC)c1=O]
ethyl 5-methoxy-2-[2-[(3R,4R)-3-methoxy-4-
(trifluoromethyl)pyrrolidin-1-yl]-3-quinolyl]-6-
methyl-4-oxo-1H-pyridine-3-carboxylate 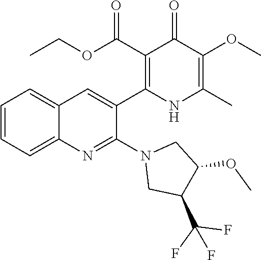 [SMILES: CCOC(=O)c1c(-c2cc3ccccc3nc2N2C[C@H](OC)[C@@H](C(F)(F)F)C2)[nH]c(C)c(OC)c1=O]
ethyl 5-methoxy-2-[2-[(3S,4R)-3-methoxy-4-
(trifluoromethyl)pyrrolidin-1-yl]-3-quinolyl]-6-
methyl-4-oxo-1H-pyridine-3-carboxylate 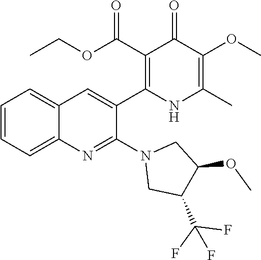 [SMILES: CCOC(=O)c1c(-c2cc3ccccc3nc2N2C[C@@H](C(F)(F)F)[C@H](OC)C2)[nH]c(C)c(OC)c1=O]
ethyl 5-methoxy-2-[2-[(3R,4S)-3-methoxy-4-
(trifluoromethyl)pyrrolidin-1-yl]-3-quinolyl]-6-
methyl-4-oxo-1H-pyridine-3-carboxylate 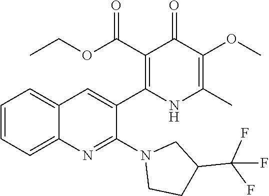 [SMILES: CCOC(=O)c1c(-c2cc3ccccc3nc2N2CCC(C(F)(F)F)C2)[nH]c(C)c(OC)c1=O]
ethyl 5-methoxy-6-methyl-4-oxo-2-[2-[3-
(trifluoromethyl)pyrrolidin-1-yl]-3-quinolyl]-1H-
pyridine-3-carboxylate 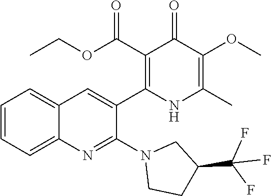 [SMILES: CCOC(=O)c1c(-c2cc3ccccc3nc2N2CC[C@H](C(F)(F)F)C2)[nH]c(C)c(OC)c1=O]
ethyl (S)-5-methoxy-6-methyl-4-oxo-2-[2-[3-
(trifluoromethyl)pyrrolidin-1-yl]-3-quinolyl]-1H-
pyridine-3-carboxylate 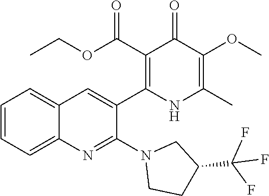 [SMILES: CCOC(=O)c1c(-c2cc3ccccc3nc2N2CC[C@@H](C(F)(F)F)C2)[nH]c(C)c(OC)c1=O]
ethyl (R)-5-methoxy-6-methyl-4-oxo-2-[2-[3-
(trifluoromethyl)pyrrolidin-1-yl]-3-quinolyl]-1H-
pyridine-3-carboxylate 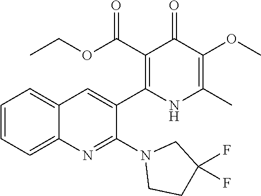 [SMILES: CCOC(=O)c1c(-c2cc3ccccc3nc2N2CCC(F)(F)C2)[nH]c(C)c(OC)c1=O]
ethyl 2-[2-(3,3-difluoropyrrolidin-1-yl)-3-
quinolyl]-5-methoxy-6-methyl-4-oxo-1H-
pyridine-3-carboxylate 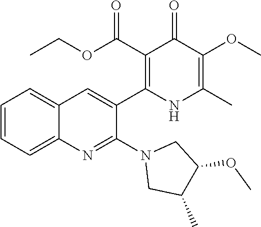 [SMILES: CCOC(=O)c1c(-c2cc3ccccc3nc2N2C[C@@H](C)[C@@H](OC)C2)[nH]c(C)c(OC)c1=O]
ethyl 5-methoxy-2-[2-[(3S,4S)-3-methoxy-4-
methyl-pyrrolidin-1-yl]-3-quinolyl]-6-methyl-
4-oxo-1H-pyridine-3-carboxylate 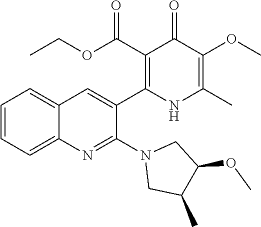 [SMILES: CCOC(=O)c1c(-c2cc3ccccc3nc2N2C[C@H](C)[C@H](OC)C2)[nH]c(C)c(OC)c1=O]
ethyl 5-methoxy-2-[2-[(3R,4R)-3-methoxy-4-
methyl-pyrrolidin-1-yl]-3-quinolyl]-6-methyl-
4-oxo-1H-pyridine-3-carboxylate 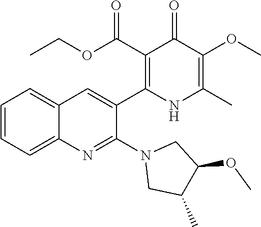 [SMILES: CCOC(=O)c1c(-c2cc3ccccc3nc2N2C[C@@H](C)[C@H](OC)C2)[nH]c(C)c(OC)c1=O]
ethyl 5-methoxy-2-[2-[(3S,4R)-3-methoxy-4-
methyl-pyrrolidin-1-yl]-3-quinolyl]-6-methyl-
4-oxo-1H-pyridine-3-carboxylate 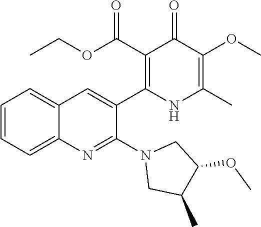 [SMILES: CCOC(=O)c1c(-c2cc3ccccc3nc2N2C[C@H](OC)[C@@H](C)C2)[nH]c(C)c(OC)c1=O]
ethyl 5-methoxy-2-[2-[(3R,4S)-3-methoxy-4-
methyl-pyrrolidin-1-yl]-3-quinolyl]-6-methyl-
4-oxo-1H-pyridine-3-carboxylate 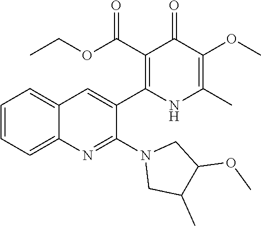 [SMILES: CCOC(=O)c1c(-c2cc3ccccc3nc2N2CC(C)C(OC)C2)[nH]c(C)c(OC)c1=O]
ethyl 5-methoxy-2-[2-[3-methoxy-4-methyl-
pyrrolidin-1-yl]-3-quinolyl]-6-methyl-4-
oxo-1H-pyridine-3-carboxylate 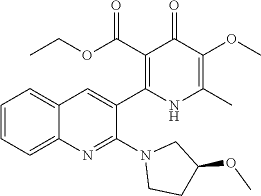 [SMILES: CCOC(=O)c1c(-c2cc3ccccc3nc2N2CC[C@H](OC)C2)[nH]c(C)c(OC)c1=O]
ethyl 5-methoxy-2-[2-[(3R)-3-methoxypyrrolidin-
1-yl]-3-quinolyl]-6-methyl-4-oxo-1H-pyridine-
3-carboxylate 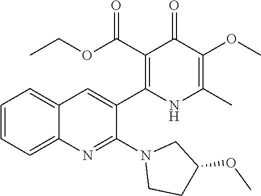 [SMILES: CCOC(=O)c1c(-c2cc3ccccc3nc2N2CC[C@@H](OC)C2)[nH]c(C)c(OC)c1=O]
ethyl 5-methoxy-2-[2-[(3S)-3-methoxypyrrolidin-
1-yl]-3-quinolyl]-6-methyl-4-oxo-1H-pyridine-
3-carboxylate 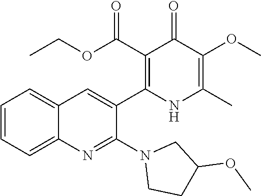 [SMILES: CCOC(=O)c1c(-c2cc3ccccc3nc2N2CCC(OC)C2)[nH]c(C)c(OC)c1=O]
ethyl 5-methoxy-2-[2-[3-methoxypyrrolidin-1-
yl]-3-quinolyl]-6-methyl-4-oxo-1H-pyridine-
3-carboxylate 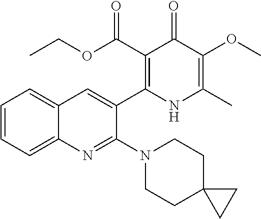 [SMILES: CCOC(=O)c1c(-c2cc3ccccc3nc2N2CCC3(CC2)CC3)[nH]c(C)c(OC)c1=O]
ethyl 2-[2-(6-azaspiro[2.5]octan-6-yl)-3-
quinolyl]-5-methoxy-6-methyl-4-oxo-1H-
pyridine-3-carboxylate 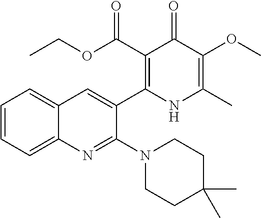 [SMILES: CCOC(=O)c1c(-c2cc3ccccc3nc2N2CCC(C)(C)CC2)[nH]c(C)c(OC)c1=O]
ethyl 2-[2-(4,4-dimethyl-1-piperidyl)-3-quinolyl]-
5-methoxy-6-methyl-4-oxo-1H-pyridine-3-
carboxylate 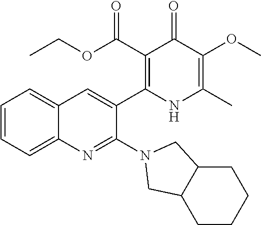 [SMILES: CCOC(=O)c1c(-c2cc3ccccc3nc2N2CC3CCCCC3C2)[nH]c(C)c(OC)c1=O]
ethyl 2-[2-(1,3,3a,4,5,6,7,7a-octahydroisoindol-2-
yl)-3-quinolyl]-5-methoxy-6-methyl-4-oxo-1H-
pyridine-3-carboxylate 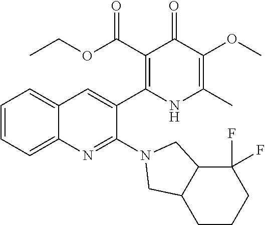 [SMILES: CCOC(=O)c1c(-c2cc3ccccc3nc2N2CC3CCCC(F)(F)C3C2)[nH]c(C)c(OC)c1=O]
ethyl 2-[2-(7,7-difluoro-3,3a,4,5,6,7a-hexahydro-
1H-isoindol-2-yl)-3-quinolyl]-5-methoxy-6-
methyl-4-oxo-1H-pyridine-3-carboxylate 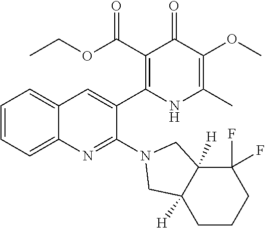 [SMILES: CCOC(=O)c1c(-c2cc3ccccc3nc2N2C[C@@H]3CCCC(F)(F)[C@@H]3C2)[nH]c(C)c(OC)c1=O]
ethyl 2-[2-(3aS,7aR)-7,7-difluoro-3,3a,4,5,6,7a-
hexahydro-1H-isoindol-2-yl)-3-quinolyl]-5-
methoxy-6-methyl-4-oxo-1H-pyridine-
3-carboxylate 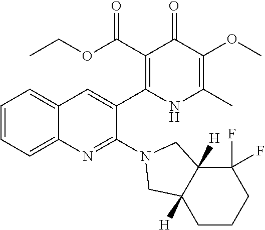 [SMILES: CCOC(=O)c1c(-c2cc3ccccc3nc2N2C[C@H]3CCCC(F)(F)[C@H]3C2)[nH]c(C)c(OC)c1=O]
ethyl 2-[2-(3aS,7aS)-7,7-difluoro-3,3a,4,5,6,7a-
hexahydro-1H-isoindol-2-yl)-3-quinolyl]-5-
methoxy-6-methyl-4-oxo-1H-pyridine-
3-carboxylate 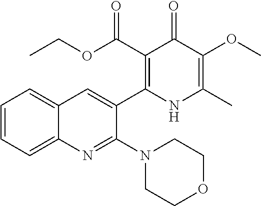 [SMILES: CCOC(=O)c1c(-c2cc3ccccc3nc2N2CCOCC2)[nH]c(C)c(OC)c1=O]
ethyl 5-methoxy-6-methyl-2-(2-morpholino-3-
quinolyl)-4-oxo-1H-pyridine-3-carboxylate 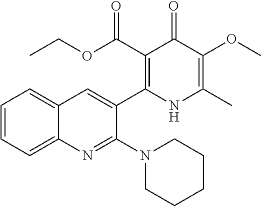 [SMILES: CCOC(=O)c1c(-c2cc3ccccc3nc2N2CCCCC2)[nH]c(C)c(OC)c1=O]
ethyl 5-methoxy-6-methyl-4-oxo-2-[2-(1-
piperidyl)-3-quinolyl)-1H-pyridine-3-carboxylate 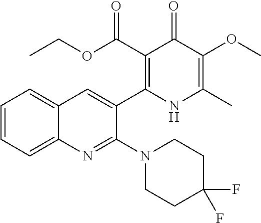 [SMILES: CCOC(=O)c1c(-c2cc3ccccc3nc2N2CCC(F)(F)CC2)[nH]c(C)c(OC)c1=O]
ethyl 5-methoxy-[2-[4,4-difluoro-1-piperidyl)-3-
quinolyl)-6-methyl-4-oxo-1H-pyridine-
3-carboxylate 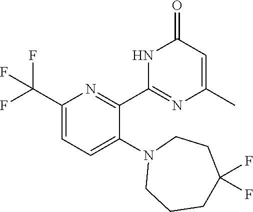 [SMILES: Cc1cc(=O)[nH]c(-c2nc(C(F)(F)F)ccc2N2CCCC(F)(F)CC2)n1]
2-[3-(4,4-difluoroazepan-1-yl)-6-
(trifluoromethyl)-2-pyridyl]-4-methyl-1H-
pyrimidin-6-one 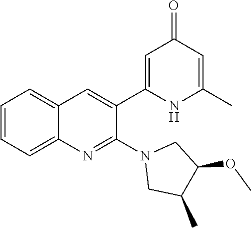 [SMILES: CO[C@@H]1CN(c2nc3ccccc3cc2-c2cc(=O)cc(C)[nH]2)C[C@@H]1C]
2-[2-[(3S,4S)-3-methoxy-4-methyl-pyrrolidin-1-
yl]-3-quinolyl]-6-methyl-1H-pyrimidin-4-one 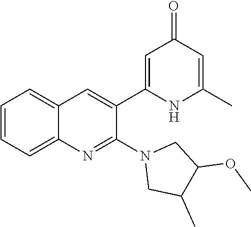 [SMILES: COC1CN(c2nc3ccccc3cc2-c2cc(=O)cc(C)[nH]2)CC1C]
2-[2-[3-methoxy-4-methyl-pyrrolidin-1-yl]-3-
quinolyl]-6-methyl-1H-pyrimidin-4-one 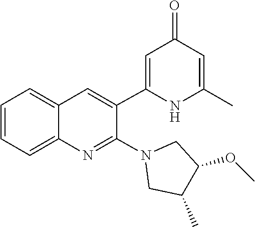 [SMILES: CO[C@H]1CN(c2nc3ccccc3cc2-c2cc(=O)cc(C)[nH]2)C[C@H]1C]
2-[2-[(3R,4R)-3-methoxy-4-methyl-pyrrolidin-1-
yl]-3-quinolyl]-6-methyl-1H-pyrimidin-4-one 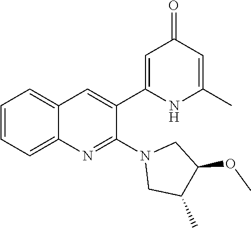 [SMILES: CO[C@@H]1CN(c2nc3ccccc3cc2-c2cc(=O)cc(C)[nH]2)C[C@H]1C]
2-[2-[(3SR,4R)-3-methoxy-4-methyl-pyrrolidin-1-
yl]-3-quinolyl]-6-methyl-1H-pyrimidin-4-one 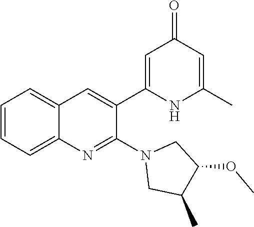 [SMILES: CO[C@H]1CN(c2nc3ccccc3cc2-c2cc(=O)cc(C)[nH]2)C[C@@H]1C]
2-[2-[(3R,4S)-3-methoxy-4-methyl-pyrrolidin-1-
yl]-3-quinolyl]-6-methyl-1H-pyrimidin-4-one 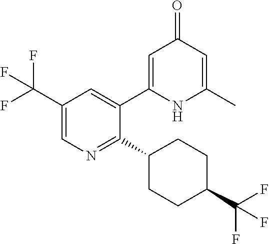 [SMILES: Cc1cc(=O)cc(-c2cc(C(F)(F)F)cnc2[C@H]2CC[C@H](C(F)(F)F)CC2)[nH]1]
6-methyl-5′-(trifluoromethyl)-2′-((1r,4r)-4-
(trifluoromethyl)cyclohexyl)-[2,3′-bipyridin]-
4(1H)-one 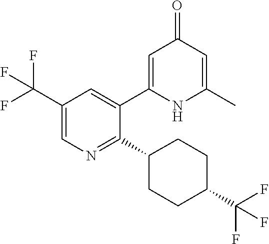 [SMILES: Cc1cc(=O)cc(-c2cc(C(F)(F)F)cnc2[C@H]2CC[C@@H](C(F)(F)F)CC2)[nH]1]
6-methyl-5′-(trifluoromethyl)-2′-((1s,4s)-4-
(trifluoromethyl)cyclohexyl)-[2,3′-bipyridin]-
4(1H)-one 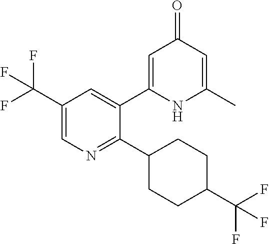 [SMILES: Cc1cc(=O)cc(-c2cc(C(F)(F)F)cnc2C2CCC(C(F)(F)F)CC2)[nH]1]
6-methyl-5′-(trifluoromethyl)-2′-(4-
(trifluoromethyl)cyclohexyl)-[2,3′-bipyridin]-
4(1H)-one 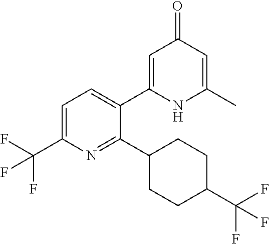 [SMILES: Cc1cc(=O)cc(-c2ccc(C(F)(F)F)nc2C2CCC(C(F)(F)F)CC2)[nH]1]
2-methyl-6-[6-(trifluoromethyl)-2-[4-
(trifluoromethyl)cyclohexyl]-3-pyridyl]-1H-
pyridin-4-one 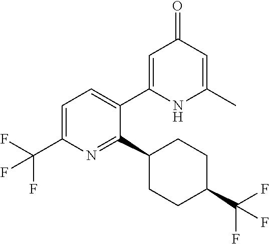 [SMILES: Cc1cc(=O)cc(-c2ccc(C(F)(F)F)nc2[C@H]2CC[C@@H](C(F)(F)F)CC2)[nH]1]
2-methyl-6-[6-(trifluoromethyl)-2-((1s,4s)-4-
(trifluoromethyl)cyclohexyl)-3-pyridyl]-1H-
pyridin-4-one 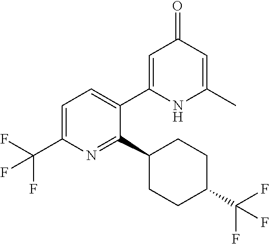 [SMILES: Cc1cc(=O)cc(-c2ccc(C(F)(F)F)nc2[C@H]2CC[C@H](C(F)(F)F)CC2)[nH]1]
2-methyl-6-[6-(trifluoromethyl)-2-((1r,4r)-4-
(trifluoromethyl)cyclohexyl)-3-pyridyl]-1H-
pyridin-4-one 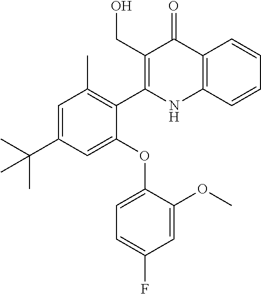 [SMILES: COc1cc(F)ccc1Oc1cc(C(C)(C)C)cc(C)c1-c1[nH]c2ccccc2c(=O)c1CO]
ethyl 2-[2-(3,4-difluoro-2-methyl-phenoxy)-3-
quinolyl]-5-fluoro-6-methyl-4-oxo-1H-
pyridine-3-carboxylate 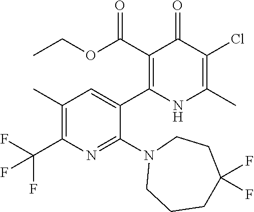 [SMILES: CCOC(=O)c1c(-c2cc(C)c(C(F)(F)F)nc2N2CCCC(F)(F)CC2)[nH]c(C)c(Cl)c1=O]
ethyl 5-chloro-2-[2-(4,4-difluoroazepan-1-yl)-5-
methyl-6-(trifluoromethyl)-3-pyridyl]-6-methyl-
4-oxo-1H-pyridine-3-carboxylate 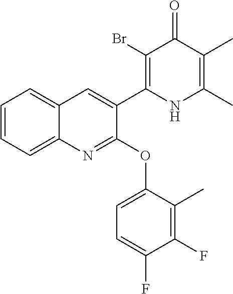 [SMILES: Cc1[nH]c(-c2cc3ccccc3nc2Oc2ccc(F)c(F)c2C)c(Br)c(=O)c1C]
3-bromo-2-[2-(3,4-difluoro-2-methyl-phenoxy)-
3-quinolyl]-5,6-dimethyl-1H-pyridin-4-one 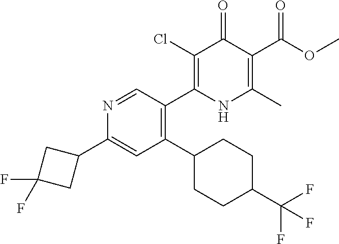 [SMILES: COC(=O)c1c(C)[nH]c(-c2cnc(C3CC(F)(F)C3)cc2C2CCC(C(F)(F)F)CC2)c(Cl)c1=O]
methyl 5-chloro-6-[6-(3,3-difluorocyclobutyl)-4-
[4-(trifluoromethyl)cyclohexyl]-3-pyridyl]-2-
methyl-4-oxo-1H-pyridine-3-carboxylate 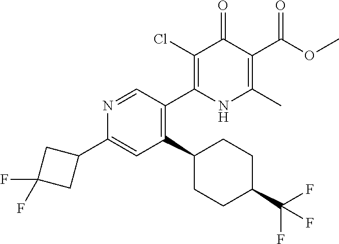 [SMILES: COC(=O)c1c(C)[nH]c(-c2cnc(C3CC(F)(F)C3)cc2[C@H]2CC[C@@H](C(F)(F)F)CC2)c(Cl)c1=O]
methyl 5-chloro-6-[6-(3,3-difluorocyclobutyl)-4-
((1s,4s)-4-(trifluoromethyl)cyclohexyl)-3-
pyridyl]-2-methyl-4-oxo-1H-pyridine-
3-carboxylate 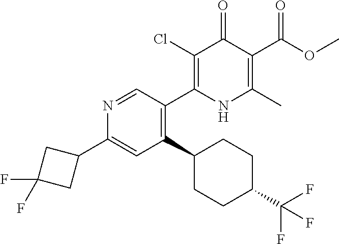 [SMILES: COC(=O)c1c(C)[nH]c(-c2cnc(C3CC(F)(F)C3)cc2[C@H]2CC[C@H](C(F)(F)F)CC2)c(Cl)c1=O]
methyl 5-chloro-6-[6-(3,3-difluorocyclobutyl)-4-
((1r,4r)-4-(trifluoromethyl)cyclohexyl)-3-
pyridyl]-2-methyl-4-oxo-1H-pyridine-
3-carboxylate 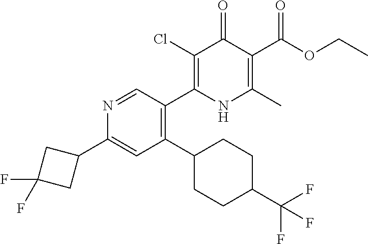 [SMILES: CCOC(=O)c1c(C)[nH]c(-c2cnc(C3CC(F)(F)C3)cc2C2CCC(C(F)(F)F)CC2)c(Cl)c1=O]
ethyl 5-chloro-6-[6-(3,3-difluorocyclobutyl)-4-[4-
(trifluoromethyl)cyclohexyl]-3-pyridyl]-2-methyl-
4-oxo-1H-pyridine-3-carboxylate 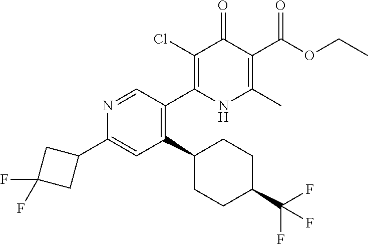 [SMILES: CCOC(=O)c1c(C)[nH]c(-c2cnc(C3CC(F)(F)C3)cc2[C@H]2CC[C@@H](C(F)(F)F)CC2)c(Cl)c1=O]
ethyl 5-chloro-6-[6-(3,3-difluorocyclobutyl)-4-
((1s,4s)-4-(trifluoromethyl)cyclohexyl]-3-
pyridyl]-2-methyl-4-oxo-1H-pyridine-
3-carboxylate 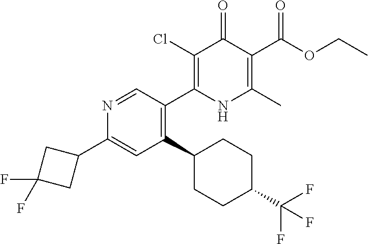 [SMILES: CCOC(=O)c1c(C)[nH]c(-c2cnc(C3CC(F)(F)C3)cc2[C@H]2CC[C@H](C(F)(F)F)CC2)c(Cl)c1=O]
ethyl 5-chloro-6-[6-(3,3-difluorocyclobutyl)-4-
((1r,4r)-4-(trifluoromethyl)cyclohexyl]-3-
pyridyl]-2-methyl-4-oxo-1H-pyridine-
3-carboxylate 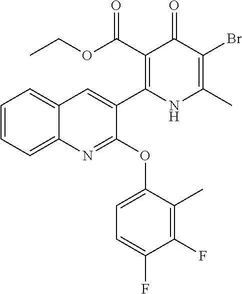 [SMILES: CCOC(=O)c1c(-c2cc3ccccc3nc2Oc2ccc(F)c(F)c2C)[nH]c(C)c(Br)c1=O]
ethyl 5-bromo-2-[2-(3,4-difluoro-2-methyl-
phenoxy)-3-quinolyl]-6-methyl-4-oxo-1H-
pyridine-3-carboxylate 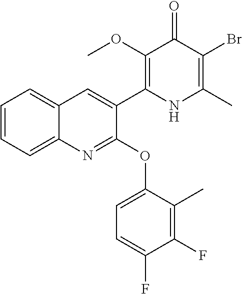 [SMILES: COc1c(-c2cc3ccccc3nc2Oc2ccc(F)c(F)c2C)[nH]c(C)c(Br)c1=O]
5-bromo-2-[2-(3,4-difluoro-2-methyl-phenoxy)-
3-quinolyl]-3-methoxy-6-methyl-1H-
pyridine-4-one 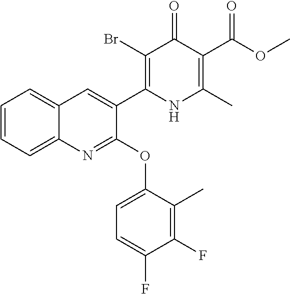 [SMILES: COC(=O)c1c(C)[nH]c(-c2cc3ccccc3nc2Oc2ccc(F)c(F)c2C)c(Br)c1=O]
methyl 5-bromo-6-[2-(3,4-difluoro-2-methyl-
phenoxy)-3-quinolyl]-2-methyl-4-oxo-1H-
pyridine-3-carboxylate 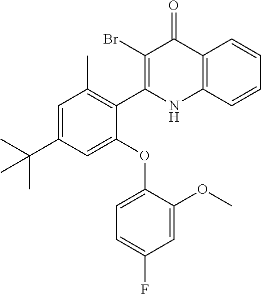 [SMILES: COc1cc(F)ccc1Oc1cc(C(C)(C)C)cc(C)c1-c1[nH]c2ccccc2c(=O)c1Br]
5-chloro-2-[2-(3,4-difluoro-2-methyl-phenoxy)-
3-quinolyl]-3-(ethoxymethyl)-6-methyl-1H-
pyridine-4-one 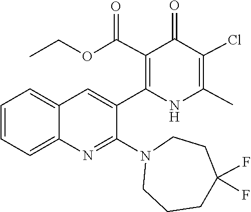 [SMILES: CCOC(=O)c1c(-c2cc3ccccc3nc2N2CCCC(F)(F)CC2)[nH]c(C)c(Cl)c1=O]
ethyl 5-chloro-2-[2-(4,4-difluoroazepan-1-yl)-3-
quinolyl]-6-methyl-4-oxo-1H-pyridine-
3-carboxylate 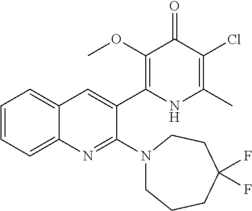 [SMILES: COc1c(-c2cc3ccccc3nc2N2CCCC(F)(F)CC2)[nH]c(C)c(Cl)c1=O]
5-chloro-2-[2-(4,4-difluoroazepan-1-yl)-3-
quinolyl]-3-methoxy-6-methyl-1H-pyridin-
4-one 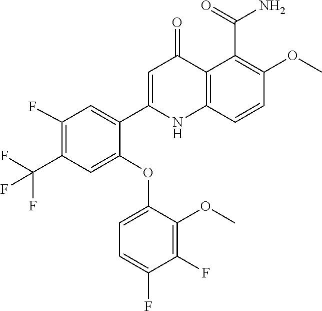 [SMILES: COc1ccc2[nH]c(-c3cc(F)c(C(F)(F)F)cc3Oc3ccc(F)c(F)c3OC)cc(=O)c2c1C(N)=O]
ethyl 5-bromo-6-[2-(3,4-difluoro-2-methyl-
phenoxy)-3-quinolyl]-2-methyl-4-oxo-1H-
pyridine-3-carboxylate 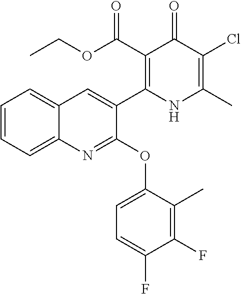 [SMILES: CCOC(=O)c1c(-c2cc3ccccc3nc2Oc2ccc(F)c(F)c2C)[nH]c(C)c(Cl)c1=O]
ethyl 5-chloro-2-[2-(3,4-difluoro-2-methyl-
phenoxy)-3-quinolyl]-6-methyl-4-oxo-1H-
pyridine-3-carboxylate 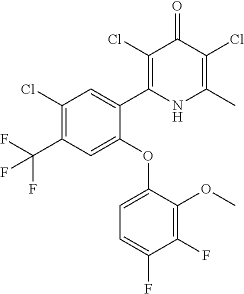 [SMILES: COc1c(Oc2cc(C(F)(F)F)c(Cl)cc2-c2[nH]c(C)c(Cl)c(=O)c2Cl)ccc(F)c1F]
3,5-dichloro-2-[5-chloro-2-(3,4-difluoro-2-
methoxy-phenoxy)-4-(trifluoromethyl)phenyl]-
6-methyl-1H-pyridin-4-one 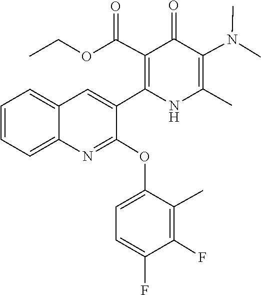 [SMILES: CCOC(=O)c1c(-c2cc3ccccc3nc2Oc2ccc(F)c(F)c2C)[nH]c(C)c(N(C)C)c1=O]
ethyl 2-[2-(3,4-difluoro-2-methyl-phenoxy)-3-
quinolyl]-5-(dimethylamino)-6-methyl-4-oxo-
1H-pyridine-3-carboxylate 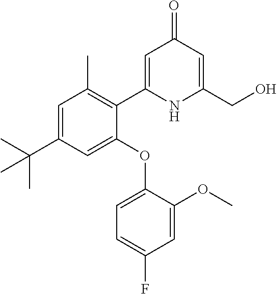 [SMILES: COc1cc(F)ccc1Oc1cc(C(C)(C)C)cc(C)c1-c1cc(=O)cc(CO)[nH]1]
2-[4-tert-butyl-2-(4-fluoro-2-methoxy-phenoxy)-
6-methyl-phenyl]-6-(hydroxymethyl)-1H-
pyridin-4-one 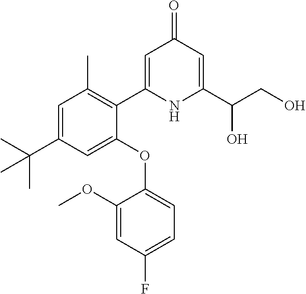 [SMILES: COc1cc(F)ccc1Oc1cc(C(C)(C)C)cc(C)c1-c1cc(=O)cc(C(O)CO)[nH]1]
2-[4-tert-butyl-2-(4-fluoro-2-methoxy-phenoxy)-
6-methyl-phenyl]-6-(1,2-dihydroxyethyl)-1H-
pyridin-4-one 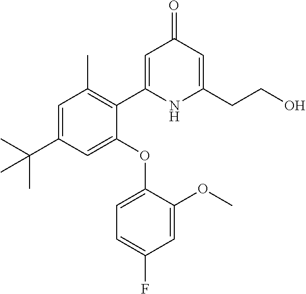 [SMILES: COc1cc(F)ccc1Oc1cc(C(C)(C)C)cc(C)c1-c1cc(=O)cc(CCO)[nH]1]
2-[4-tert-butyl-2-(4-fluoro-2-methoxy-phenoxy)-
6-methyl-phenyl]-6-(2-hydroxyethyl)-1H-
pyridin-4-one 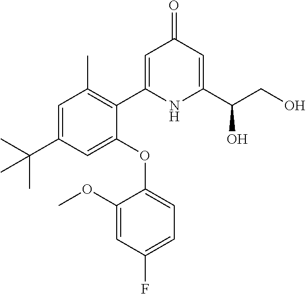 [SMILES: COc1cc(F)ccc1Oc1cc(C(C)(C)C)cc(C)c1-c1cc(=O)cc([C@H](O)CO)[nH]1]
(S)-2-[4-tert-butyl-2-(4-fluoro-2-methoxy-
phenoxy)-6-methyl-phenyl]-6-(1,2-
dihydroxyethyl)-1H-pyridin-4-one 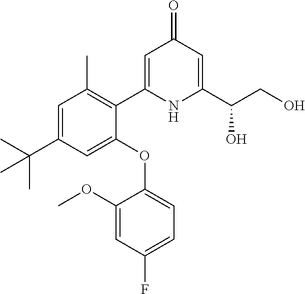 [SMILES: COc1cc(F)ccc1Oc1cc(C(C)(C)C)cc(C)c1-c1cc(=O)cc([C@@H](O)CO)[nH]1]
(R)-2-[4-tert-butyl-2-(4-fluoro-2-methoxy-
phenoxy)-6-methyl-phenyl]-6-(1,2-
dihydroxyethyl)-1H-pyridin-4-one 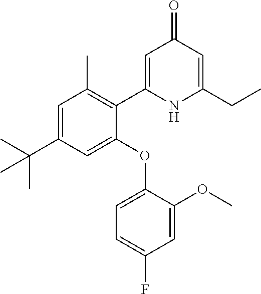 [SMILES: CCc1cc(=O)cc(-c2c(C)cc(C(C)(C)C)cc2Oc2ccc(F)cc2OC)[nH]1]
2-[4-tert-butyl-2-(4-fluoro-2-methoxy-phenoxy)-
6-methyl-phenyl]-6-ethyl-1H-pyridin-4-one 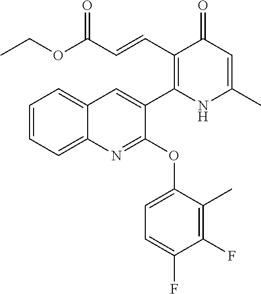 [SMILES: CCOC(=O)/C=C/c1c(-c2cc3ccccc3nc2Oc2ccc(F)c(F)c2C)[nH]c(C)cc1=O]
ethyl (E)-3-[2-[2-(3,4-difluoro-2-methyl-
phenoxy)-3-quinolyl]-6-methyl-4-oxo-1H-
pyridine-3-carboxylate 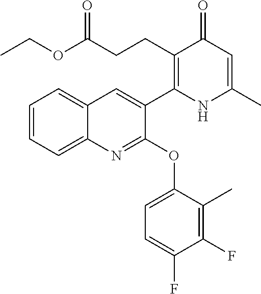 [SMILES: CCOC(=O)CCc1c(-c2cc3ccccc3nc2Oc2ccc(F)c(F)c2C)[nH]c(C)cc1=O]
ethyl 3-[2-[2-(3,4-difluoro-2-methyl-phenoxy)-3-
quinolyl]-6-methyl-4-oxo-1H-pyridine-3-
carboxylate 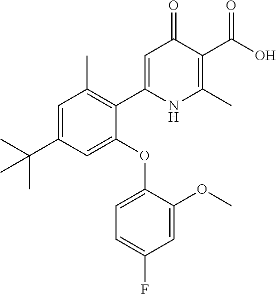 [SMILES: COc1cc(F)ccc1Oc1cc(C(C)(C)C)cc(C)c1-c1cc(=O)c(C(=O)O)c(C)[nH]1]
6-[4-tert-butyl-2-(4-fluoro-2-methoxy-phenoxy)-
6-methyl-phenyl]-2-methyl-4-oxo-1H-pyridine-
3-carboxylic acid 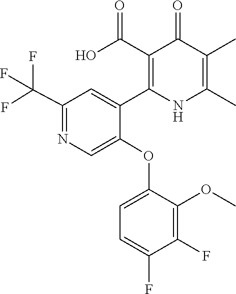 [SMILES: COc1c(Oc2cnc(C(F)(F)F)cc2-c2[nH]c(C)c(C)c(=O)c2C(=O)O)ccc(F)c1F]
2-[5-(3,4-difluoro-2-methoxy-phenoxy)-2-
(trifluoromethyl)-4-pyridyl]-5,6-dimethyl-4-oxo-
1H-pyridine-3-carboxylic acid 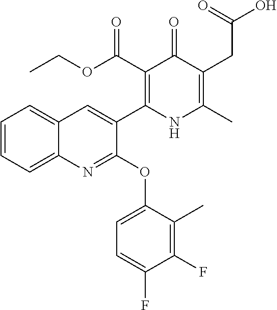 [SMILES: CCOC(=O)c1c(-c2cc3ccccc3nc2Oc2ccc(F)c(F)c2C)[nH]c(C)c(CC(=O)O)c1=O]
2-[6-[2-(3,4-difluoro-2-methoxy-phenoxy)-3-
quinolyl]-5-ethoxycarbonyl-2-methyl-4-oxo-1H-
pyridin-3-yl]acetic acid 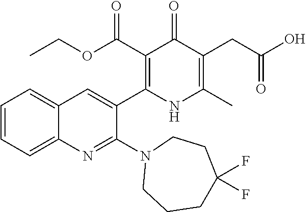 [SMILES: CCOC(=O)c1c(-c2cc3ccccc3nc2N2CCCC(F)(F)CC2)[nH]c(C)c(CC(=O)O)c1=O]
2-[6-[2-(4,4-difluoroazepan-1-yl)-3-quinolyl]-5-
ethoxycarbonyl-2-methyl-4-oxo-1H-pyridin-3-
yl]acetic acid 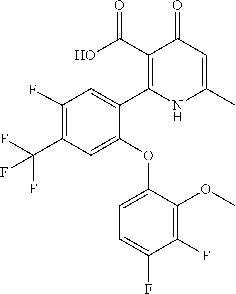 [SMILES: COc1c(Oc2cc(C(F)(F)F)c(F)cc2-c2[nH]c(C)cc(=O)c2C(=O)O)ccc(F)c1F]
2-[2-(3,4-difluoro-2-methoxy-phenoxy)-5-fluoro-
4-(trifluoromethyl)phenyl]-6-methyl-4-oxo-
1H-pyridine-3-carboxylic acid 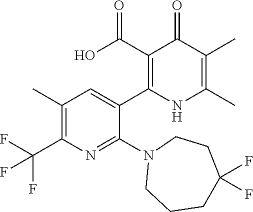 [SMILES: Cc1cc(-c2[nH]c(C)c(C)c(=O)c2C(=O)O)c(N2CCCC(F)(F)CC2)nc1C(F)(F)F]
2-[2-(4,4-difluoroazepan-1-yl)-5-methyl-6-
(trifluoromethyl)-3-pyridyl]-5,6-dimethyl-4-oxo-
1H-pyridine-3-carboxylic acid 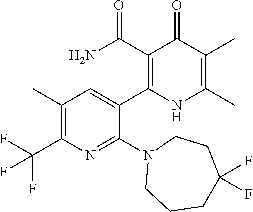 [SMILES: Cc1cc(-c2[nH]c(C)c(C)c(=O)c2C(N)=O)c(N2CCCC(F)(F)CC2)nc1C(F)(F)F]
2-[2-(4,4-difluoroazepan-1-yl)-5-methyl-6-
(trifluoromethyl)-3-pyridyl]-5,6-dimethyl-4-oxo-
1H-pyridine-3-carboxylic acid 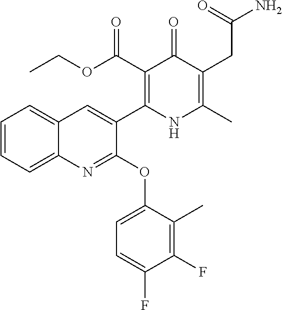 [SMILES: CCOC(=O)c1c(-c2cc3ccccc3nc2Oc2ccc(F)c(F)c2C)[nH]c(C)c(CC(N)=O)c1=O]
ethyl 5-(2-amino-2-oxo-ethyl)-2-[2-(3,4-difluoro-
2-methyl-phenoxy)-3-quinolyl]-6-methyl-4-oxo-
1H-pyridine-3-carboxylate 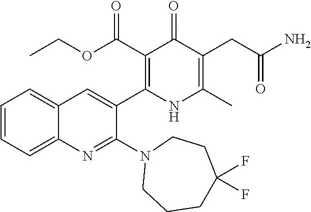 [SMILES: CCOC(=O)c1c(-c2cc3ccccc3nc2N2CCCC(F)(F)CC2)[nH]c(C)c(CC(N)=O)c1=O]
ethyl 5-(2-amino-2-oxo-ethyl)-2-[2-(4,4-
difluoroazepan-1-yl)-3-quinolyl]-6-methyl-
4-oxo-1H-pyridine-3-carboxylate 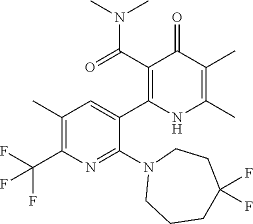 [SMILES: Cc1cc(-c2[nH]c(C)c(C)c(=O)c2C(=O)N(C)C)c(N2CCCC(F)(F)CC2)nc1C(F)(F)F]
2-[2-(4,4-difluoroazepan-1-yl)-5-methyl-6-
(trifluoromethyl)-3-pyridyl]-N,N,5,6-tetramethyl-
4-oxo-1H-pyridine-3-carboxylic acid 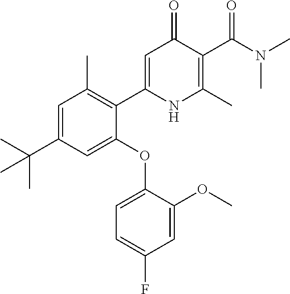 [SMILES: COc1cc(F)ccc1Oc1cc(C(C)(C)C)cc(C)c1-c1cc(=O)c(C(=O)N(C)C)c(C)[nH]1]
6-[4-tert-butyl-2-(4-fluoro-2-methoxy-phenoxy)-
6-methyl-phenyl]-N,N,2-trimethyl-4-oxo-1H-
pyridine-3-carboxamide 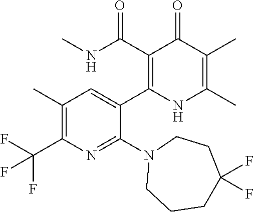 [SMILES: CNC(=O)c1c(-c2cc(C)c(C(F)(F)F)nc2N2CCCC(F)(F)CC2)[nH]c(C)c(C)c1=O]
2-[2-(4,4-difluoroazepan-1-yl)-5-methyl-6-
(trifluoromethyl)-3-pyridyl]-N,5,6-trimethyl-4-
oxo-1H-pyridine-3-carboxamide 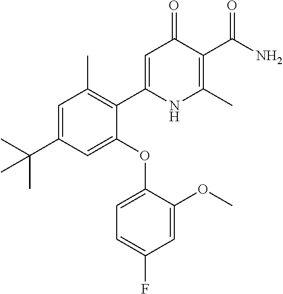 [SMILES: COc1cc(F)ccc1Oc1cc(C(C)(C)C)cc(C)c1-c1cc(=O)c(C(N)=O)c(C)[nH]1]
6-[4-tert-butyl-2-(4-fluoro-2-methoxy-phenoxy)-
6-methyl-phenyl]-2-methyl-4-oxo-1H-pyridine-
3-carboxamide 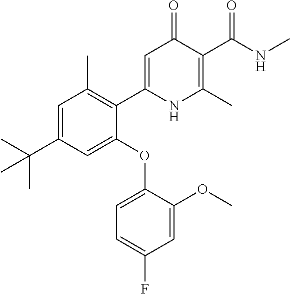 [SMILES: CNC(=O)c1c(C)[nH]c(-c2c(C)cc(C(C)(C)C)cc2Oc2ccc(F)cc2OC)cc1=O]
6-[4-tert-butyl-2-(4-fluoro-2-methoxy-phenoxy)-
6-methyl-phenyl]-N,2-dimethyl-4-oxo-1H-
pyridine-3-carboxamide 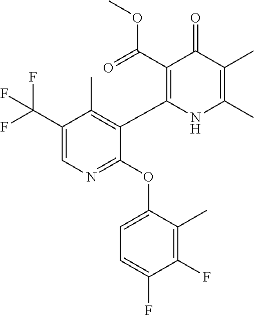 [SMILES: COC(=O)c1c(-c2c(Oc3ccc(F)c(F)c3C)ncc(C(F)(F)F)c2C)[nH]c(C)c(C)c1=O]
methyl 2-[2-(3,4-difluoro-2-methyl-phenoxy)-4-
methyl-5-(trifluoromethyl)-3-pyridyl]-5,6-
dimethyl-4-oxo-1H-pyridine-3-carboxylate 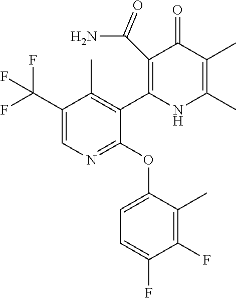 [SMILES: Cc1[nH]c(-c2c(Oc3ccc(F)c(F)c3C)ncc(C(F)(F)F)c2C)c(C(N)=O)c(=O)c1C]
2-[2-(3,4-difluoro-2-methyl-phenoxy)-4-methyl-
5-(trifluoromethyl)-3-pyridyl]-5,6-dimethyl-4-
oxo-1H-pyridine-3-carboxamide 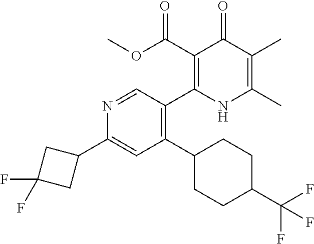 [SMILES: COC(=O)c1c(-c2cnc(C3CC(F)(F)C3)cc2C2CCC(C(F)(F)F)CC2)[nH]c(C)c(C)c1=O]
methyl 2-[6-(3,3-difluorocyclobutyl)-4-[4-
(trifluoromethyl)cyclohexyl]-3-pyridyl]-5,6-
dimethyl-4-oxo-1H-pyridine-3-carboxylate 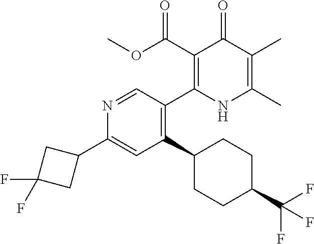 [SMILES: COC(=O)c1c(-c2cnc(C3CC(F)(F)C3)cc2[C@H]2CC[C@@H](C(F)(F)F)CC2)[nH]c(C)c(C)c1=O]
methyl 2-[6-(3,3-difluorocyclobutyl)-4-((1s,4s)-4-
(trifluoromethyl)cyclohexyl)-3-pyridyl]-5,6-
dimethyl-4-oxo-1H-pyridine-3-carboxylate 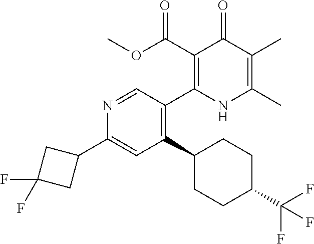 [SMILES: COC(=O)c1c(-c2cnc(C3CC(F)(F)C3)cc2[C@H]2CC[C@H](C(F)(F)F)CC2)[nH]c(C)c(C)c1=O]
methyl 2-[6-(3,3-difluorocyclobutyl)-4-((1r,4r)-4-
(trifluoromethyl)cyclohexyl)-3-pyridyl]-5,6-
dimethyl-4-oxo-1H-pyridine-3-carboxylate 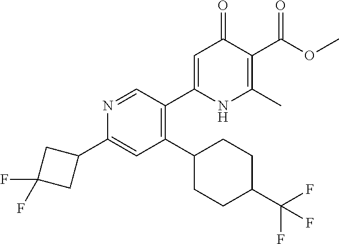 [SMILES: COC(=O)c1c(C)[nH]c(-c2cnc(C3CC(F)(F)C3)cc2C2CCC(C(F)(F)F)CC2)cc1=O]
methyl 6-[6-(3,3-difluorocyclobutyl)-4-[4-
(trifluoromethyl)cyclohexyl]-3-pyridyl]-2-
methyl-4-oxo-1H-pyridine-3-carboxylate 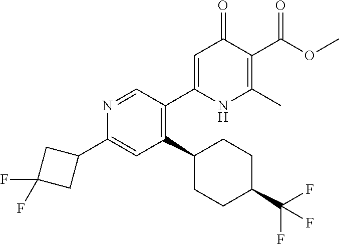 [SMILES: COC(=O)c1c(C)[nH]c(-c2cnc(C3CC(F)(F)C3)cc2[C@H]2CC[C@@H](C(F)(F)F)CC2)cc1=O]
methyl 6-[6-(3,3-difluorocyclobutyl)-4-((1s,4s)-4-
(trifluoromethyl)cyclohexyl)-3-pyridyl]-2-methyl-
4-oxo-1H-pyridine-3-carboxylate 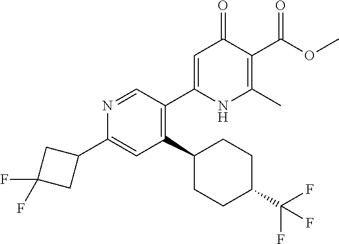 [SMILES: COC(=O)c1c(C)[nH]c(-c2cnc(C3CC(F)(F)C3)cc2[C@H]2CC[C@H](C(F)(F)F)CC2)cc1=O]
methyl 6-[6-(3,3-difluorocyclobutyl)-4-((1r,4r)-4-
(trifluoromethyl)cyclohexyl)-3-pyridyl]-2-methyl-
4-oxo-1H-pyridine-3-carboxylate 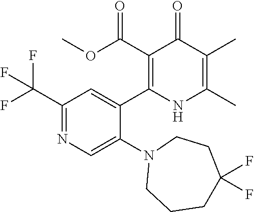 [SMILES: COC(=O)c1c(-c2cc(C(F)(F)F)ncc2N2CCCC(F)(F)CC2)[nH]c(C)c(C)c1=O]
methyl 2-[5-(4,4-difluoroazepan-1-yl)-2-
(trifluoromethyl)-4-pyridyl]-5,6-dimethyl-4-
oxo-1H-pyridine-3-carboxamide 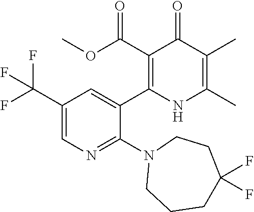 [SMILES: COC(=O)c1c(-c2cc(C(F)(F)F)cnc2N2CCCC(F)(F)CC2)[nH]c(C)c(C)c1=O]
methyl 2-[2-(4,4-difluoroazepan-1-yl)-5-
(trifluoromethyl)-3-pyridyl]-5,6-dimethyl-4-
oxo-1H-pyridine-3-carboxamide 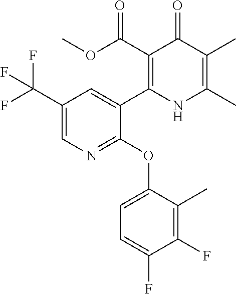 [SMILES: COC(=O)c1c(-c2cc(C(F)(F)F)cnc2Oc2ccc(F)c(F)c2C)[nH]c(C)c(C)c1=O]
methyl 2-[2-(3,4-difluoro-2-methyl-phenoxy)-5-
(trifluoromethyl)-3-pyridyl]-5,6-dimethyl-4-oxo-
1H-pyridine-3-carboxylate 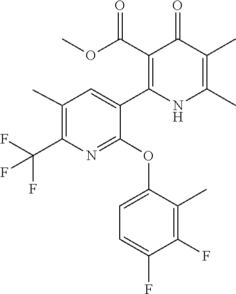 [SMILES: COC(=O)c1c(-c2cc(C)c(C(F)(F)F)nc2Oc2ccc(F)c(F)c2C)[nH]c(C)c(C)c1=O]
methyl 2-[2-(3,4-difluoro-2-methyl-phenoxy)-
5-methyl-6-(trifluoromethyl)-3-pyridyl]-5,6-
dimethyl-4-oxo-1H-pyridine-3-carboxylate 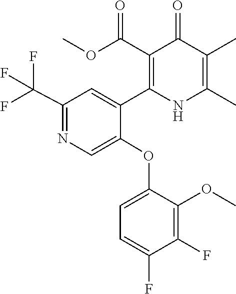 [SMILES: COC(=O)c1c(-c2cc(C(F)(F)F)ncc2Oc2ccc(F)c(F)c2OC)[nH]c(C)c(C)c1=O]
methyl 2-[5-(3,4-difluoro-2-methoxy-phenoxy)-2-
(trifluoromethyl)-4-pyridyl]-5,6-dimethyl-4-oxo-
1H-pyridine-3-carboxylate 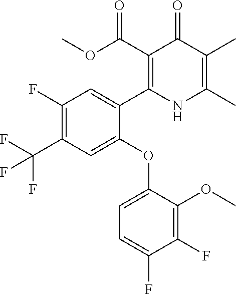 [SMILES: COC(=O)c1c(-c2cc(F)c(C(F)(F)F)cc2Oc2ccc(F)c(F)c2OC)[nH]c(C)c(C)c1=O]
methyl 2-[2-(3,4-difluoro-2-methoxy-phenoxy)-5-
fluoro-4-(trifluoromethyl)phenyl]-5,6-dimethyl-
4-oxo-1H-pyridine-3-carboxylate 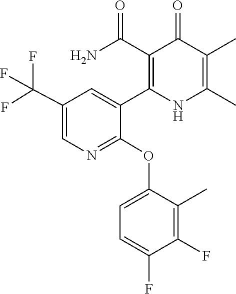 [SMILES: Cc1[nH]c(-c2cc(C(F)(F)F)cnc2Oc2ccc(F)c(F)c2C)c(C(N)=O)c(=O)c1C]
2-[2-(3,4-difluoro-2-methoxy-phenoxy)-5-
(trifluoromethyl)-3-pyridyl]-5,6-dimethyl-
4-oxo-1H-pyridine-3-carboxamide 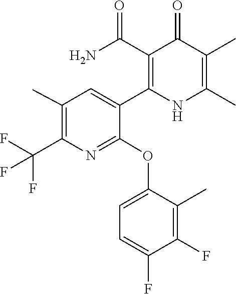 [SMILES: Cc1cc(-c2[nH]c(C)c(C)c(=O)c2C(N)=O)c(Oc2ccc(F)c(F)c2C)nc1C(F)(F)F]
2-[2-(3,4-difluoro-2-methoxy-phenoxy)-5-methyl-
6-(trifluoromethyl)-3-pyridyl]-5,6-dimethyl-4-
oxo-1H-pyridine-3-carboxamide 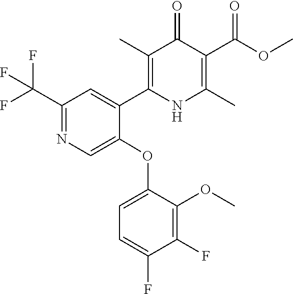 [SMILES: COC(=O)c1c(C)[nH]c(-c2cc(C(F)(F)F)ncc2Oc2ccc(F)c(F)c2OC)c(C)c1=O]
methyl 6-[5-(3,4-difluoro-2-methoxy-phenoxy)-2-
(trifluoromethyl)-4-pyridyl]-2,5-dimethyl-4-
oxo-1H-pyridine-3-carboxylate 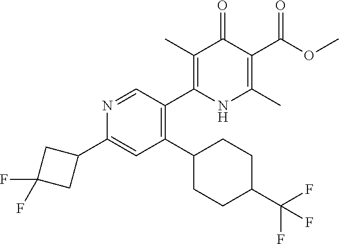 [SMILES: COC(=O)c1c(C)[nH]c(-c2cnc(C3CC(F)(F)C3)cc2C2CCC(C(F)(F)F)CC2)c(C)c1=O]
methyl 6-[6-(3,3-difluorocyclobutyl)-4-[4-
(trifluoromethyl)cyclohexyl]-3-pyridyl]-2,5-
dimethyl-4-oxo-1H-pyridine-3-carboxylate 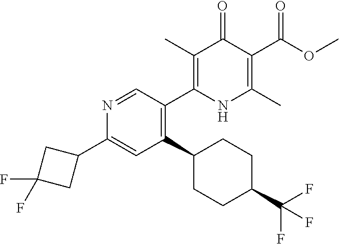 [SMILES: COC(=O)c1c(C)[nH]c(-c2cnc(C3CC(F)(F)C3)cc2[C@H]2CC[C@@H](C(F)(F)F)CC2)c(C)c1=O]
methyl 6-[6-(3,3-difluorocyclobutyl)-4-((1s,4s)-4-
(trifluoromethyl)cyclohexyl)-3-pyridyl]-2,5-
dimethyl-4-oxo-1H-pyridine-3-carboxylate 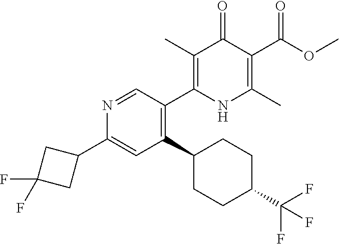 [SMILES: COC(=O)c1c(C)[nH]c(-c2cnc(C3CC(F)(F)C3)cc2[C@H]2CC[C@H](C(F)(F)F)CC2)c(C)c1=O]
methyl 6-[6-(3,3-difluorocyclobutyl)-4-((1r,4r)-4-
(trifluoromethyl)cyclohexyl)-3-pyridyl]-2,5-
dimethyl-4-oxo-1H-pyridine-3-carboxylate 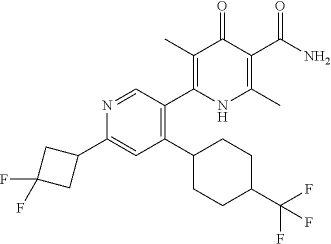 [SMILES: Cc1[nH]c(-c2cnc(C3CC(F)(F)C3)cc2C2CCC(C(F)(F)F)CC2)c(C)c(=O)c1C(N)=O]
6-[6-(3,3-difluorocyclobutyl)-4-[4-
(trifluoromethyl)cyclohexyl]-3-pyridyl]-2,5-
dimethyl-4-oxo-1H-pyridine-3-carboxamide 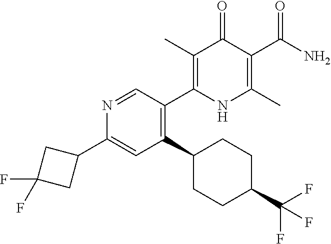 [SMILES: Cc1[nH]c(-c2cnc(C3CC(F)(F)C3)cc2[C@H]2CC[C@@H](C(F)(F)F)CC2)c(C)c(=O)c1C(N)=O]
6-[6-(3,3-difluorocyclobutyl)-4-((1s,4s)-4-
(trifluoromethyl)cyclohexyl)-3-pyridyl]-2,5-
dimethyl-4-oxo-1H-pyridine-3-carboxamide 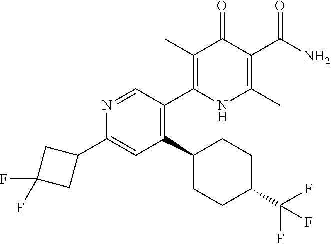 [SMILES: Cc1[nH]c(-c2cnc(C3CC(F)(F)C3)cc2[C@H]2CC[C@H](C(F)(F)F)CC2)c(C)c(=O)c1C(N)=O]
6-[6-(3,3-difluorocyclobutyl)-4-((1r,4r)-4-
(trifluoromethyl)cyclohexyl)-3-pyridyl]-2,5-
dimethyl-4-oxo-1H-pyridine-3-carboxamide 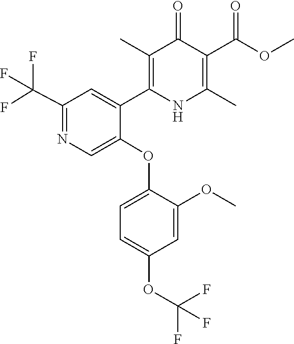 [SMILES: COC(=O)c1c(C)[nH]c(-c2cc(C(F)(F)F)ncc2Oc2ccc(OC(F)(F)F)cc2OC)c(C)c1=O]
methyl 6-[5-[2-methoxy-4-
(trifluoromethoxy)phenoxy]-2-(trifluoromethyl)-
4-pyridyl]-2,5-dimethyl-4-oxo-1H-pyridine-3-
carboxylate 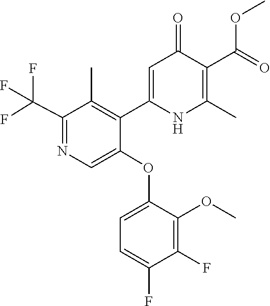 [SMILES: COC(=O)c1c(C)[nH]c(-c2c(Oc3ccc(F)c(F)c3OC)cnc(C(F)(F)F)c2C)cc1=O]
methyl 6-[5-(3,4-difluoro-2-methoxy-phenoxy)-3-
methyl-2-(trifluoromethyl)-4-pyridyl]-2-methyl-4-
oxo-1H-pyridine-3-carboxylate 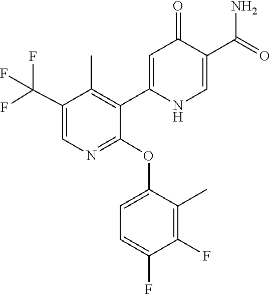 [SMILES: Cc1c(Oc2ncc(C(F)(F)F)c(C)c2-c2cc(=O)c(C(N)=O)c[nH]2)ccc(F)c1F]
6-[2-(3,4-difluoro-2-methyl-phenoxy)-4-methyl-
5-(trifluoromethyl)-3-pyridyl]-4-oxo-1H-
pyridine-3-carboxylate 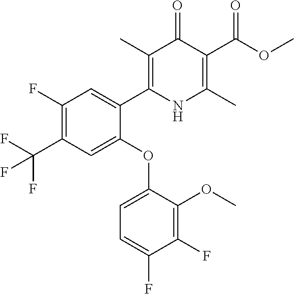 [SMILES: COC(=O)c1c(C)[nH]c(-c2cc(F)c(C(F)(F)F)cc2Oc2ccc(F)c(F)c2OC)c(C)c1=O]
methyl 6-[2-(3,4-difluoro-2-methoxy-phenoxy)-5
fluoro-4-(trifluoromethyl)phenyl]-2,5-dimethyl-
4-oxo-1H-pyridine-3-carboxylate 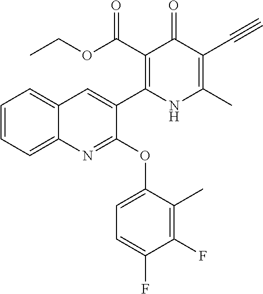 [SMILES: C#Cc1c(C)[nH]c(-c2cc3ccccc3nc2Oc2ccc(F)c(F)c2C)c(C(=O)OCC)c1=O]
ethyl 2-[2-(3,4-difluoro-2-methyl-phenoxy)-3-
quinolyl]-5-ethynyl-6-methyl-4-oxo-1H-
pyridine-3-carboxylate 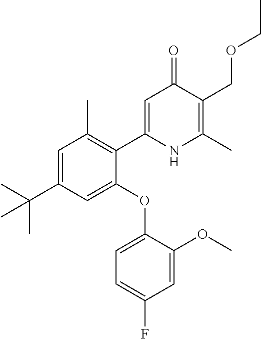 [SMILES: CCOCc1c(C)[nH]c(-c2c(C)cc(C(C)(C)C)cc2Oc2ccc(F)cc2OC)cc1=O]
6-[4-tert-butyl-2-(4-fluoro-2-methoxy-phenoxy)-
6-methyl-phenyl]-3-(ethoxymethyl)-2-methyl-1H-
pyridine-4-one 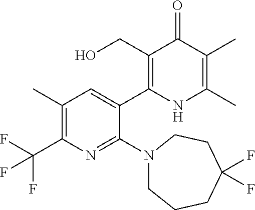 [SMILES: Cc1cc(-c2[nH]c(C)c(C)c(=O)c2CO)c(N2CCCC(F)(F)CC2)nc1C(F)(F)F]
2-[1-(4,4-difluoroazepan-1-yl)-5-methyl-6-
(trifluoromethyl)-3-pyridyl]-3-(hydroxymethyl)-
5,6-dimethyl-1H-pyridine-4-one 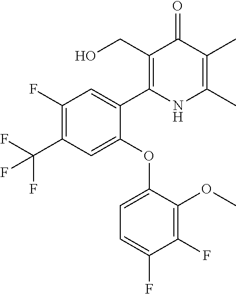 [SMILES: COc1c(Oc2cc(C(F)(F)F)c(F)cc2-c2[nH]c(C)c(C)c(=O)c2CO)ccc(F)c1F]
2-[2-(3,4-difluoro-2-methoxy-phenoxy)-5-fluoro-
4-(trifluoromethyl)phenyl]-3-(hydroxymethyl)-
5,6-dimethyl-1H-pyridine-4-one 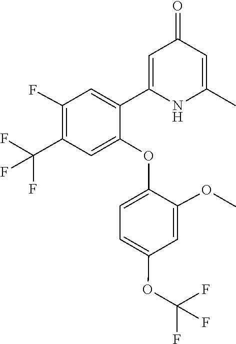 [SMILES: COc1cc(OC(F)(F)F)ccc1Oc1cc(C(F)(F)F)c(F)cc1-c1cc(=O)cc(C)[nH]1]
2-[5-fluoro-2-[2-methoxy-4-
(trifluoromethoxy)phenoxy-4-
(trifluoromethyl)phenyl]-6-methyl-1H-
pyridine-4-one 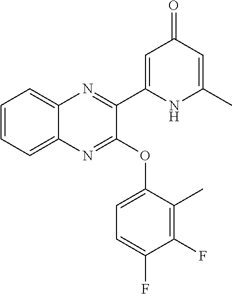 [SMILES: Cc1cc(=O)cc(-c2nc3ccccc3nc2Oc2ccc(F)c(F)c2C)[nH]1]
2-[3-(3,4-difluoro-2-methyl-phenoxy)quinoxalin-
2-yl]-6-methyl-1H-pyridine-4-one 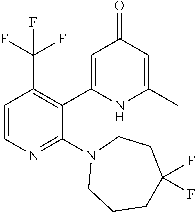 [SMILES: Cc1cc(=O)cc(-c2c(C(F)(F)F)ccnc2N2CCCC(F)(F)CC2)[nH]1]
2-[2-(4,4-difluoroazepan-1-yl)-4-
(trifluoromethyl)-3-pyridyl]-6-methyl-1H-
pyridine-4-one 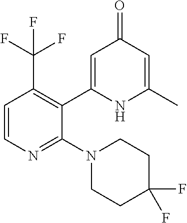 [SMILES: Cc1cc(=O)cc(-c2c(C(F)(F)F)ccnc2N2CCC(F)(F)CC2)[nH]1]
2-[2-(4,4-difluoro-1-piperidyl)-4-
(trifluoromethyl)-3-pyridyl]-6-methyl-1H-
pyridin-4-one 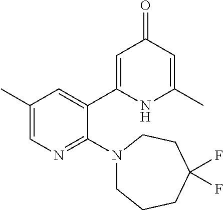 [SMILES: Cc1cnc(N2CCCC(F)(F)CC2)c(-c2cc(=O)cc(C)[nH]2)c1]
2-[2-(4,4-difluoroazepan-1-yl)-5-methyl-3-
pyridyl]-6-methyl-1H-pyridine-4-one 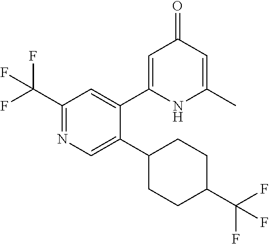 [SMILES: Cc1cc(=O)cc(-c2cc(C(F)(F)F)ncc2C2CCC(C(F)(F)F)CC2)[nH]1]
2-methyl-6-[2-(trifluoromethyl)-5-[4-
(trifluoromethyl)cyclohexyl]-4-pyridyl]-1H-
pyridine-4-one 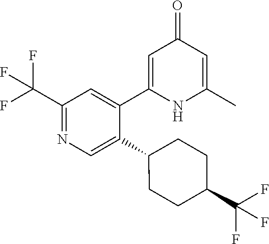 [SMILES: Cc1cc(=O)cc(-c2cc(C(F)(F)F)ncc2[C@H]2CC[C@H](C(F)(F)F)CC2)[nH]1]
2-methyl-6-[2-(trifluoromethyl)-5-(1r4r)-4-
(trifluoromethyl)cyclohexyl)-4-pyridyl]-1H-
pyridine-4-one 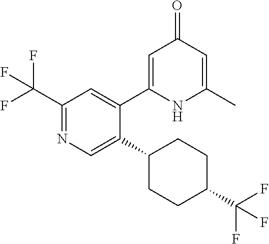 [SMILES: Cc1cc(=O)cc(-c2cc(C(F)(F)F)ncc2[C@H]2CC[C@@H](C(F)(F)F)CC2)[nH]1]
2-methyl-6-[2-(trifluoromethyl)-5-(1s,4s)-4-
(trifluoromethyl)cyclohexyl)-4-pyridyl]-1H-
pyridine-4-one 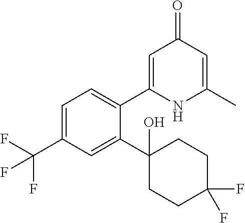 [SMILES: Cc1cc(=O)cc(-c2ccc(C(F)(F)F)cc2C2(O)CCC(F)(F)CC2)[nH]1]
2-[2-(4,4-difluoro-1-hydroxy-cyclohexyl)-4-
(trifluoromethyl)phenyl]-6-methyl-1H-
pyridine-4-one 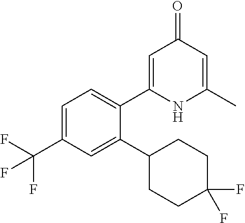 [SMILES: Cc1cc(=O)cc(-c2ccc(C(F)(F)F)cc2C2CCC(F)(F)CC2)[nH]1]
2-[2-(4,4-difluorocyclohexyl)-4-
(trifluoromethyl)phenyl]-6-methyl-1H-
pyridine-4-one
and 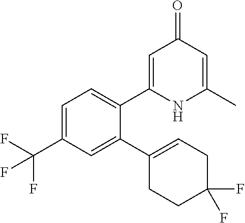 [SMILES: Cc1cc(=O)cc(-c2ccc(C(F)(F)F)cc2C2=CCC(F)(F)CC2)[nH]1]
2-[2-(4,4-difluorocyclohexen-1-yl)-4-
(trifluoromethyl)phenyl]-6-methyl-1H-
pyridin-4-one
or a pharmaceutically acceptable salt thereof.

56. (canceled)

57. A compound selected from: 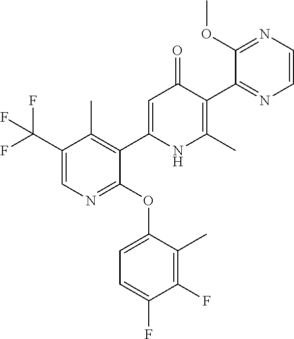 [SMILES: COc1nccnc1-c1c(C)[nH]c(-c2c(Oc3ccc(F)c(F)c3C)ncc(C(F)(F)F)c2C)cc1=O]
6-[2-(3,4-difluoro-2-methyl-phenoxy)-4-methyl-
5-(trifluoromethyl)-3-pyridyl]-3-(3-methoxy-
pyrazin-2-yl)-2-methyl-1H-pyridin-4-one, 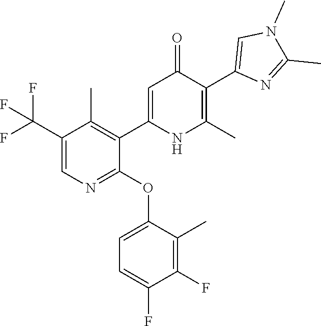 [SMILES: Cc1[nH]c(-c2c(Oc3ccc(F)c(F)c3C)ncc(C(F)(F)F)c2C)cc(=O)c1-c1cn(C)c(C)n1]
6-[2-(3,4-difluoro-2-methyl-phenoxy)-4-methyl-
5-(trifluoromethyl)-3-pyridyl]-3-(1,2-dimethyl-
imidazol-4-yl)-2-methyl-1H-pyridin-4-one, 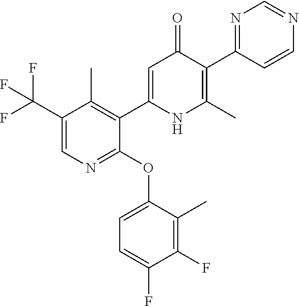 [SMILES: Cc1[nH]c(-c2c(Oc3ccc(F)c(F)c3C)ncc(C(F)(F)F)c2C)cc(=O)c1-c1ccncn1]
6-[2-(3,4-difluoro-2-methyl-phenoxy)-4-methyl-
5-(trifluoromethyl)-3-pyridyl]-2-methyl-3-
pyrimidin-4-yl-1H-pyridin-4-one, 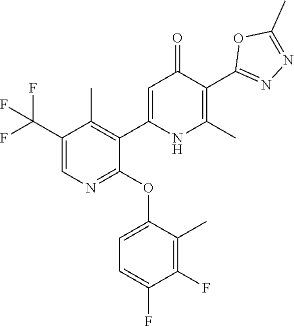 [SMILES: Cc1nnc(-c2c(C)[nH]c(-c3c(Oc4ccc(F)c(F)c4C)ncc(C(F)(F)F)c3C)cc2=O)o1]
6-[2-(3,4-difluoro-2-methyl-phenoxy)-4-methyl-
5-(trifluoromethyl)-3-pyridyl]-2-methyl-3-(5-
methyl-1,3,4-oxadiazol-2-yl)-1H-pyridin-4-one, 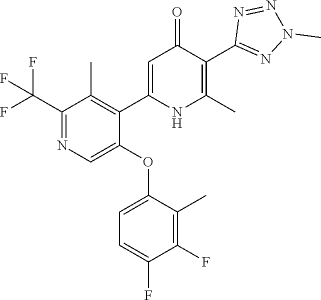 [SMILES: Cc1[nH]c(-c2c(Oc3ccc(F)c(F)c3C)cnc(C(F)(F)F)c2C)cc(=O)c1-c1nnn(C)n1]
6-[5-(3,4-difluoro-2-methyl-phenoxy)-3-methyl-
2-(trifluoromethyl)-4-pyridyl]-2-methyl-3-(2-
methyltetrazol-5-yl)-1H-pyridin-4-one, 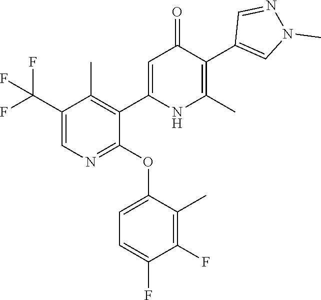 [SMILES: Cc1[nH]c(-c2c(Oc3ccc(F)c(F)c3C)ncc(C(F)(F)F)c2C)cc(=O)c1-c1cnn(C)c1]
6-[2-(3,4-difluoro-2-methyl-phenoxy)-4-methyl-
5-(trifluoromethyl)-3-pyridyl]-2-methyl-3-(1-
methylpyrazol-4-yl)-1H-pyridin-4-one, 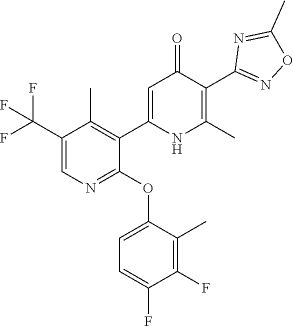 [SMILES: Cc1nc(-c2c(C)[nH]c(-c3c(Oc4ccc(F)c(F)c4C)ncc(C(F)(F)F)c3C)cc2=O)no1]
6-[2-(3,4-difluoro-2-methyl-phenoxy)-4-methyl-
5-(trifluoromethyl)-3-pyridyl]-2-methyl-3-(5-
methyl-1,2,4-oxadiazol-3-yl)-1H-pyridin-4-one, 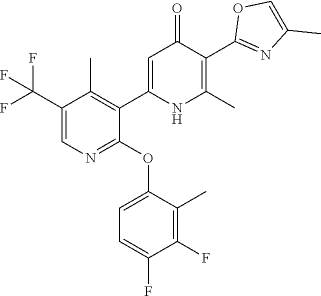 [SMILES: Cc1coc(-c2c(C)[nH]c(-c3c(Oc4ccc(F)c(F)c4C)ncc(C(F)(F)F)c3C)cc2=O)n1]
6-[2-(3,4-difluoro-2-methyl-phenoxy)-4-methyl-
5-(trifluoromethyl)-3-pyridyl]-2-methyl-3-(4-
methyloxazol-2-yl)-1H-pyridin-4-one, 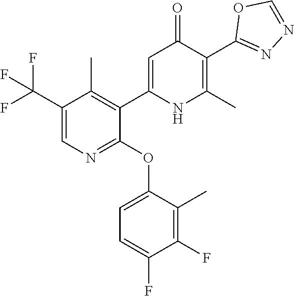 [SMILES: Cc1[nH]c(-c2c(Oc3ccc(F)c(F)c3C)ncc(C(F)(F)F)c2C)cc(=O)c1-c1nnco1]
6-[2-(3,4-difluoro-2-methyl-phenoxy)-4-methyl-
5-(trifluoromethyl)-3-pyridyl]-2-methyl-3-(1,3,4-
oxadiazol-2-yl)-1H-pyridin-4-one, 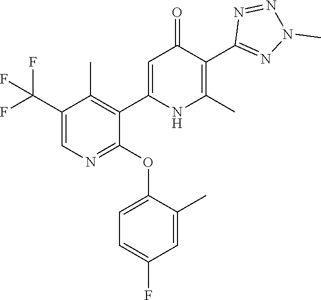 [SMILES: Cc1cc(F)ccc1Oc1ncc(C(F)(F)F)c(C)c1-c1cc(=O)c(-c2nnn(C)n2)c(C)[nH]1]
6-[2-(4-fluoro-2-methyl-phenoxy)-4-methyl-5
(trifluoromethyl)-3-pyridyl]-2-methyl-3-(2-methyl-
tetrazol-5-yl)-1H-pyridin-4-one, 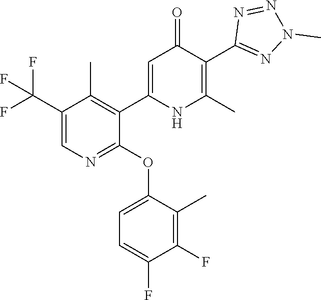 [SMILES: Cc1[nH]c(-c2c(Oc3ccc(F)c(F)c3C)ncc(C(F)(F)F)c2C)cc(=O)c1-c1nnn(C)n1]
6-[2-(3,4-difluoro-2-methyl-phenoxy)-4-methyl-5
(trifluoromethyl)-3-pyridyl]-2-methyl-3-(2-methyl-
tetrazol-5-yl)-1H-pyridin-4-one, 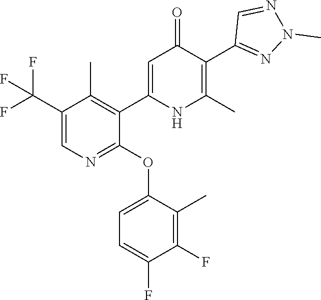 [SMILES: Cc1[nH]c(-c2c(Oc3ccc(F)c(F)c3C)ncc(C(F)(F)F)c2C)cc(=O)c1-c1cnn(C)n1]
6-[2-(3,4-difluoro-2-methyl-phenoxy)-4-methyl-5
(trifluoromethyl)-3-pyridyl]-2-methyl-3-(2-methyl-
triazol-4-yl)-1H-pyridin-4-one, 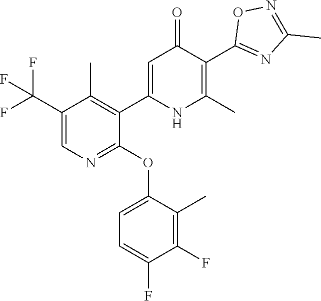 [SMILES: Cc1noc(-c2c(C)[nH]c(-c3c(Oc4ccc(F)c(F)c4C)ncc(C(F)(F)F)c3C)cc2=O)n1]
6-[2-(3,4-difluoro-2-methyl-phenoxy)-4-methyl-5
(trifluoromethyl)-3-pyridyl]-2-methyl-3-(3-methyl-
1,2,4-oxadiazol-5-yl)-1H-pyridin-4-one, 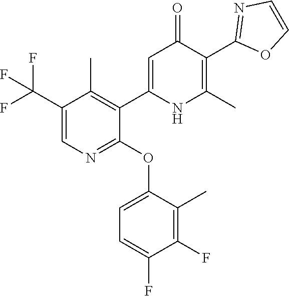 [SMILES: Cc1[nH]c(-c2c(Oc3ccc(F)c(F)c3C)ncc(C(F)(F)F)c2C)cc(=O)c1-c1ncco1]
6-[2-(3,4-difluoro-2-methyl-phenoxy)-4-methyl-
5-(trifluoromethyl)-3-pyridyl]-2-methyl-3-
oxazol-2-yl-1H-pyridin-4-one, 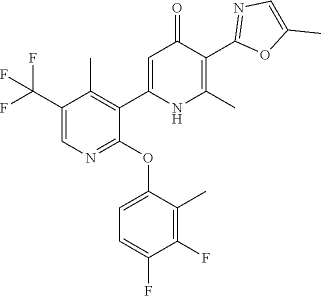 [SMILES: Cc1cnc(-c2c(C)[nH]c(-c3c(Oc4ccc(F)c(F)c4C)ncc(C(F)(F)F)c3C)cc2=O)o1]
6-[2-(3,4-difluoro-2-methyl-phenoxy)-4-methyl-
5-(trifluoromethyl)-3-pyridyl]-2-methyl-3-(5-
methyloxazol-2-yl)-1H-pyridin-4-one, 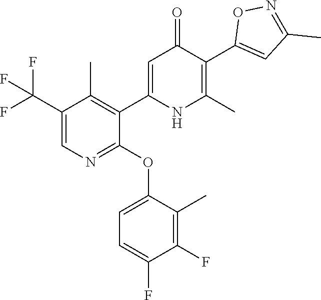 [SMILES: Cc1cc(-c2c(C)[nH]c(-c3c(Oc4ccc(F)c(F)c4C)ncc(C(F)(F)F)c3C)cc2=O)on1]
6-[2-(3,4-difluoro-2-methyl-phenoxy)-4-methyl-
5-(trifluoromethyl)-3-pyridyl]-2-methyl-3-(3-
methylisoxazol-5-yl)-1H-pyridin-4-one, 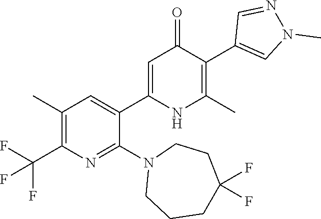 [SMILES: Cc1cc(-c2cc(=O)c(-c3cnn(C)c3)c(C)[nH]2)c(N2CCCC(F)(F)CC2)nc1C(F)(F)F]
6-[2-(4,4-difluoroazepan-1-yl)-5-methyl-6-(trifluoro-
methyl)-3-pyridyl]-2-methyl-3-(1-methylpyrazol-
4-yl)-1H-pyridin-4-one, 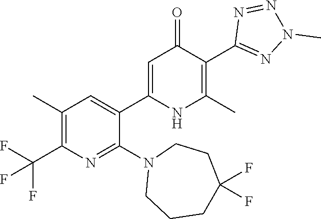 [SMILES: Cc1cc(-c2cc(=O)c(-c3nnn(C)n3)c(C)[nH]2)c(N2CCCC(F)(F)CC2)nc1C(F)(F)F]
6-[2-(4,4-difluoroazepan-1-yl)-5-methyl-6-(trifluoro-
methyl)-3-pyridyl]-2-methyl-3-(2-methyltetrazol-
5-yl)-1H-pyridin-4-one, 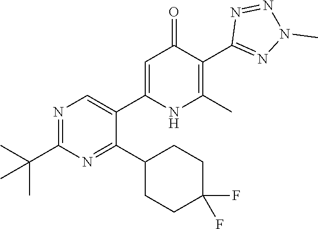 [SMILES: Cc1[nH]c(-c2cnc(C(C)(C)C)nc2C2CCC(F)(F)CC2)cc(=O)c1-c1nnn(C)n1]
6-[2-tert-butyl-4-(4,4-difluorocyclohexyl)pyrimi-
din-5-yl]-2-methyl-3-(2-methyltetrazol-5-yl)-1H-
pyridin-4-one, 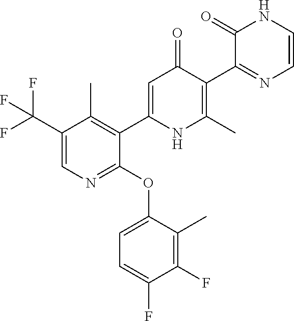 [SMILES: Cc1[nH]c(-c2c(Oc3ccc(F)c(F)c3C)ncc(C(F)(F)F)c2C)cc(=O)c1-c1ncc[nH]c1=O]
3-[6-[2-(3,4-difluoro-2-methyl-phenoxy)-4-
methyl-5-(trifluoromethyl)-3-pyridyl]-2-methyl-
4-oxo-1H-pyridin-3-yl]-1H-pyrazin-2-one, 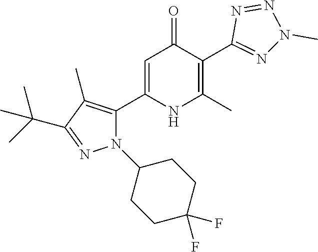 [SMILES: Cc1[nH]c(-c2c(C)c(C(C)(C)C)nn2C2CCC(F)(F)CC2)cc(=O)c1-c1nnn(C)n1]
6-[5-tert-butyl-2-(4,4-difluorocyclohexyl)-4-methyl-
pyrazol-3-yl]-2-methyl-3-(2-methyltetrazol-5-yl)-
1H-pyridin-4-one, 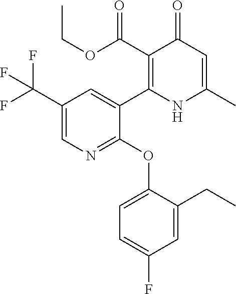 [SMILES: CCOC(=O)c1c(-c2cc(C(F)(F)F)cnc2Oc2ccc(F)cc2CC)[nH]c(C)cc1=O]
ethyl 2-[2-(2-ethyl-4-fluoro-phenoxy)-
5-(trifluoromethyl)-3-pyridyl]-6-meth-
yl-4-oxo-1H-pyridine-3-carboxylate, 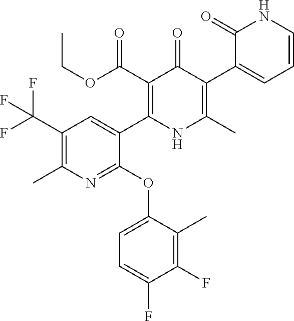 [SMILES: CCOC(=O)c1c(-c2cc(C(F)(F)F)c(C)nc2Oc2ccc(F)c(F)c2C)[nH]c(C)c(-c2ccc[nH]c2=O)c1=O]
ethyl 2-[2-(3,4-difluoro-2-methyl-phenoxy)-6-
methyl-5-(trifluoromethyl)-3-pyridyl]-6-meth-
yl-4-oxo-5-(2-oxo-1H-pyridin-3-yl)-1H-
pyridine-3-carboxylate, 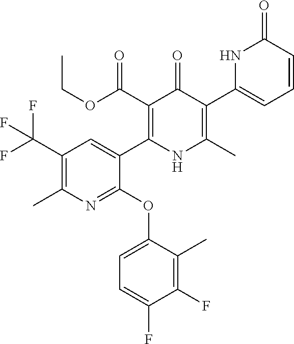 [SMILES: CCOC(=O)c1c(-c2cc(C(F)(F)F)c(C)nc2Oc2ccc(F)c(F)c2C)[nH]c(C)c(-c2cccc(=O)[nH]2)c1=O]
ethyl 2-[2-(3,4-difluoro-2-methyl-phenoxy)-6-
methyl-5-(trifluoromethyl)-3-pyridyl]-6-meth-
yl-4-oxo-5-(6-oxo-1H-pyridin-2-yl)-1H-
pyridine-3-carboxylate, 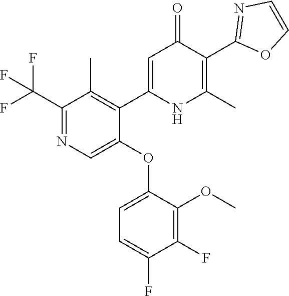 [SMILES: COc1c(Oc2cnc(C(F)(F)F)c(C)c2-c2cc(=O)c(-c3ncco3)c(C)[nH]2)ccc(F)c1F]
6-[5-(3,4-difluoro-2-methoxy-phenoxy)-3-
methyl-2-(trifluoromethyl)-4-pyridyl]-2-meth-
yl-3-oxazol-2-yl-1H-pyridin-4-one, 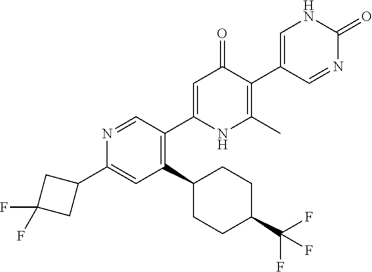 [SMILES: Cc1[nH]c(-c2cnc(C3CC(F)(F)C3)cc2[C@H]2CC[C@@H](C(F)(F)F)CC2)cc(=O)c1-c1cnc(=O)[nH]c1]
5-[6-[6-(3,3-difluorocyclobutyl)-4-[4-(trifluoromethyl)cyclo-
hexyl]-3-pyridyl]-2-methyl-4-oxo-1H-pyridin-3-yl]-1H-
pyrimidin-2-one, 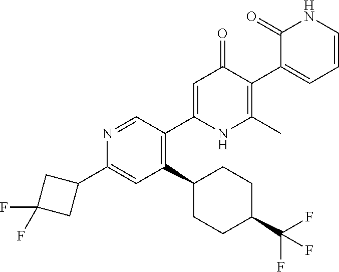 [SMILES: Cc1[nH]c(-c2cnc(C3CC(F)(F)C3)cc2[C@H]2CC[C@@H](C(F)(F)F)CC2)cc(=O)c1-c1ccc[nH]c1=O]
6-[6-(3,3-difluorocyclobutyl)-4-[4-(trifluoromethyl)-
cyclohexyl]-3-pyridyl]-2-methyl-3-(2-oxo-1H-pyridin-
3-yl)-1H-pyridin-4-one, 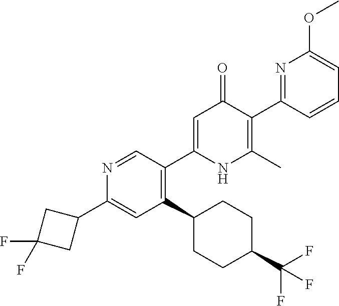 [SMILES: COc1cccc(-c2c(C)[nH]c(-c3cnc(C4CC(F)(F)C4)cc3[C@H]3CC[C@@H](C(F)(F)F)CC3)cc2=O)n1]
6-[6-(3,3-difluorocyclobutyl)-4-[4-(trifluoromethyl)-
cyclohexyl]-3-pyridyl]-3-(6-methoxy-2-pyridyl)-2-
methyl-1H-pyridin-4-one, 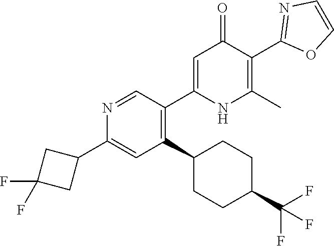 [SMILES: Cc1[nH]c(-c2cnc(C3CC(F)(F)C3)cc2[C@H]2CC[C@@H](C(F)(F)F)CC2)cc(=O)c1-c1ncco1]
6-[6-(3,3-difluorocyclobutyl)-4-[4-(trifluoromethyl)-
cyclohexyl]-3-pyridyl]-2-methyl-3-oxazol-2-yl-1H-
pyridin-4-one, 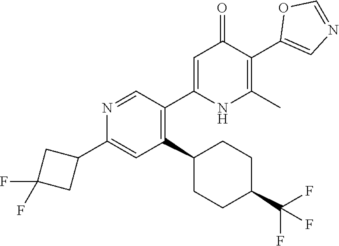 [SMILES: Cc1[nH]c(-c2cnc(C3CC(F)(F)C3)cc2[C@H]2CC[C@@H](C(F)(F)F)CC2)cc(=O)c1-c1cnco1]
6-[6-(3,3-difluorocyclobutyl)-4-[4-(trifluoromethyl)-
cyclohexyl]-3-pyridyl]-2-methyl-3-oxazol-5-yl-1H-
pyridin-4-one, 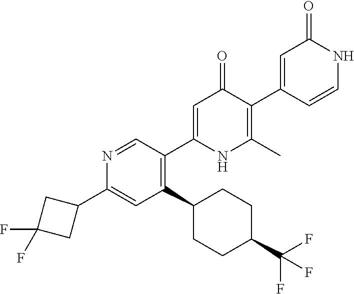 [SMILES: Cc1[nH]c(-c2cnc(C3CC(F)(F)C3)cc2[C@H]2CC[C@@H](C(F)(F)F)CC2)cc(=O)c1-c1cc[nH]c(=O)c1]
6-[6-(3,3-difluorocyclobutyl)-4-[4-(trifluoromethyl)cyclo-
hexyl]-3-pyridyl]-2-methyl-3-(2-oxo-1H-pyridin-4-yl)-
1H-pyridin-4-one, 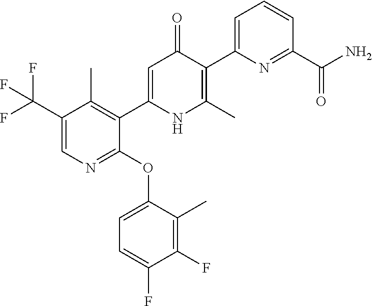 [SMILES: Cc1[nH]c(-c2c(Oc3ccc(F)c(F)c3C)ncc(C(F)(F)F)c2C)cc(=O)c1-c1cccc(C(N)=O)n1]
6-[6-[2-(3,4-difluoro-2-methyl-phenoxy)-4-methyl-5-
(trifluoromethyl)-3-pyridyl]-2-methyl-4-oxo-1H-
pyridin-3-yl]pyridine-2-carboxamide, 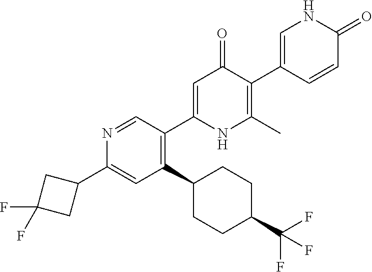 [SMILES: Cc1[nH]c(-c2cnc(C3CC(F)(F)C3)cc2[C@H]2CC[C@@H](C(F)(F)F)CC2)cc(=O)c1-c1ccc(=O)[nH]c1]
6-[6-(3,3-difluorocyclobutyl)-4-[4-(trifluoromethyl)cyclo-
hexyl]-3-pyridyl]-2-methyl-3-(6-oxo-1H-pyridin-3-yl)-
1H-pyridin-4-one, 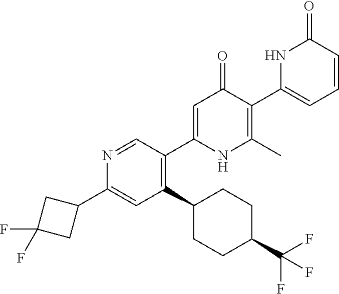 [SMILES: Cc1[nH]c(-c2cnc(C3CC(F)(F)C3)cc2[C@H]2CC[C@@H](C(F)(F)F)CC2)cc(=O)c1-c1cccc(=O)[nH]1]
6-[6-(3,3-difluorocyclobutyl)-4-[4-(trifluoromethyl)-
cyclohexyl]-3-pyridyl]-2-methyl-3-(6-oxo-1H-pyridin-
2-yl)-1H-pyridin-4-one, 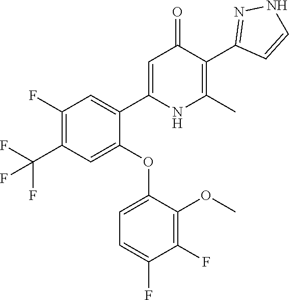 [SMILES: COc1c(Oc2cc(C(F)(F)F)c(F)cc2-c2cc(=O)c(-c3cc[nH]n3)c(C)[nH]2)ccc(F)c1F]
6-[2-(3,4-difluoro-2-methoxy-phenoxy)-5-
fluoro-4-(trifluoromethyl)phenyl]-2-methyl-
3-(1H-pyrazol-3-yl)-1H-pyridin-4-one, 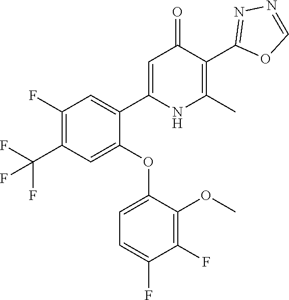 [SMILES: COc1c(Oc2cc(C(F)(F)F)c(F)cc2-c2cc(=O)c(-c3nnco3)c(C)[nH]2)ccc(F)c1F]
6-[2-(3,4-difluoro-2-methoxy-phenoxy)-5-
fluoro-4-(trifluoromethyl)phenyl]-2-methyl-
3-(1,3,4-oxadiazol-2-yl)-1H-pyridin-4-one, 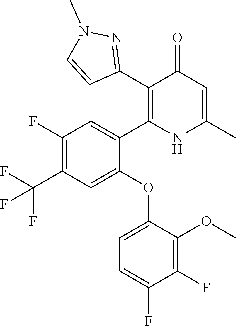 [SMILES: COc1c(Oc2cc(C(F)(F)F)c(F)cc2-c2[nH]c(C)cc(=O)c2-c2ccn(C)n2)ccc(F)c1F]
2-[2-(3,4-difluoro-2-methoxy-phen-
oxy)-5-fluoro-4-(trifluoromethyl)-
phenyl]-6-methyl-3-(1-methylpyr-
azol-3-yl)-1H-pyridin-4-one, 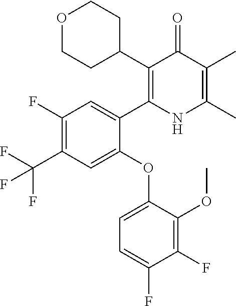 [SMILES: COc1c(Oc2cc(C(F)(F)F)c(F)cc2-c2[nH]c(C)c(C)c(=O)c2C2CCOCC2)ccc(F)c1F]
2-[2-(3,4-difluoro-2-methoxy-phen-
oxy)-5-fluoro-4-(trifluoromethyl)-
phenyl]-5,6-dimethyl-3-tetrahydro-
pyran-4-yl-1H-pyridin-4-one, 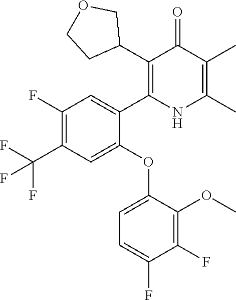 [SMILES: COc1c(Oc2cc(C(F)(F)F)c(F)cc2-c2[nH]c(C)c(C)c(=O)c2C2CCOC2)ccc(F)c1F]
2-[2-(3,4-difluoro-2-methoxy-phen-
oxy)-5-fluoro-4-(trifluoromethyl)-
phenyl]-5,6-dimethyl-3-tetrahydro-
furan-3-yl-1H-pyridin-4-one, 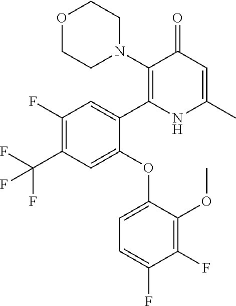 [SMILES: COc1c(Oc2cc(C(F)(F)F)c(F)cc2-c2[nH]c(C)cc(=O)c2N2CCOCC2)ccc(F)c1F]
2-[2-(3,4-difluoro-2-methoxy-phen-
oxy)-5-fluoro-4-(trifluoromethyl)-
phenyl]-6-methyl-3-morpholino-1H-
pyridin-4-one, 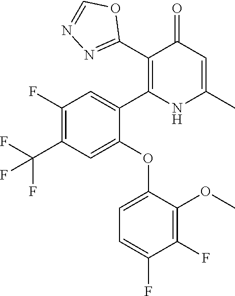 [SMILES: COc1c(Oc2cc(C(F)(F)F)c(F)cc2-c2[nH]c(C)cc(=O)c2-c2nnco2)ccc(F)c1F]
2-[2-(3,4-difluoro-2-methoxy-phen-
oxy)-5-fluoro-4-(trifluoromethyl)-
phenyl]-6-methyl-3-(1,3,4-oxadiazol-
2-yl)-1H-pyridin-4-one, 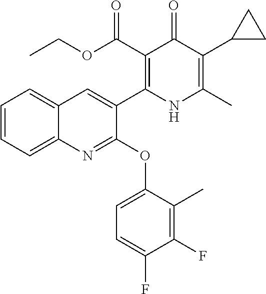 [SMILES: CCOC(=O)c1c(-c2cc3ccccc3nc2Oc2ccc(F)c(F)c2C)[nH]c(C)c(C2CC2)c1=O]
ethyl 5-cyclopropyl-2-[2-(3,4-difluoro-2-
methyl-phenoxy)-3-quinolyl]-6-methyl-4-
oxo-1H-pyridine-3-carboxylate, 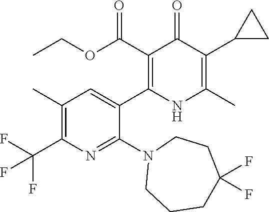 [SMILES: CCOC(=O)c1c(-c2cc(C)c(C(F)(F)F)nc2N2CCCC(F)(F)CC2)[nH]c(C)c(C2CC2)c1=O]
ethyl 5-cyclopropyl-2-[2-(4,4-difluoroaze-
pan-1-yl)-5-methyl-6-(trifluoromethyl)-
3-pyridyl]-6-methyl-4-oxo-1H-pyridine-
3-carboxylate, 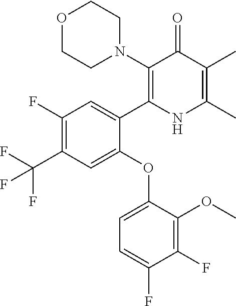 [SMILES: COc1c(Oc2cc(C(F)(F)F)c(F)cc2-c2[nH]c(C)c(C)c(=O)c2N2CCOCC2)ccc(F)c1F]
2-[2-(3,4-difluoro-2-methoxy-phen-
oxy)-5-fluoro-4-(trifluoromethyl)-
phenyl]-5,6-dimethyl-3-morpholino-
1H-pyridin-4-one, 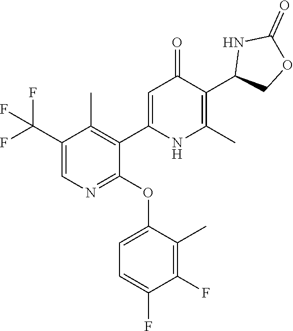 [SMILES: Cc1[nH]c(-c2c(Oc3ccc(F)c(F)c3C)ncc(C(F)(F)F)c2C)cc(=O)c1[C@@H]1COC(=O)N1]
(R)-4-[6-[2-(3,4-difluoro-2-methyl-phenoxy)-4-
methyl-5-(trifluoromethyl)-3-pyridyl]-2-methyl-
4-oxo-1H-pyridin-3-yl]oxazolidin-2-one, 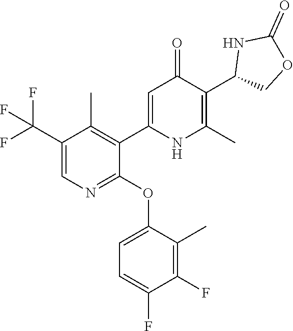 [SMILES: Cc1[nH]c(-c2c(Oc3ccc(F)c(F)c3C)ncc(C(F)(F)F)c2C)cc(=O)c1[C@H]1COC(=O)N1]
(S)-4-[6-[2-(3,4-difluoro-2-methyl-phenoxy)-4-
methyl-5-(trifluoromethyl)-3-pyridyl]-2-methyl-
4-oxo-1H-pyridin-3-yl]oxazolidin-2-one, 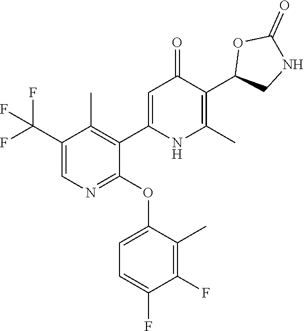 [SMILES: Cc1[nH]c(-c2c(Oc3ccc(F)c(F)c3C)ncc(C(F)(F)F)c2C)cc(=O)c1[C@@H]1CNC(=O)O1]
(R)-5-[6-[2-(3,4-difluoro-2-methyl-phenoxy)-4-
methyl-5-(trifluoromethyl)-3-pyridyl]-2-methyl-
4-oxo-1H-pyridin-3-yl]oxazolidin-2-one, 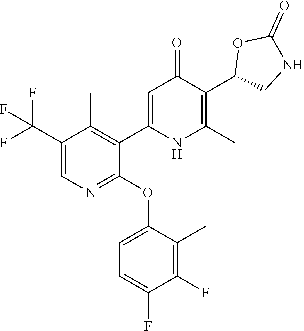 [SMILES: Cc1[nH]c(-c2c(Oc3ccc(F)c(F)c3C)ncc(C(F)(F)F)c2C)cc(=O)c1[C@H]1CNC(=O)O1]
(S)-5-[6-[2-(3,4-difluoro-2-methyl-phenoxy)-4-
methyl-5-(trifluoromethyl)-3-pyridyl]-2-methyl-
4-oxo-1H-pyridin-3-yl]oxazolidin-2-one, 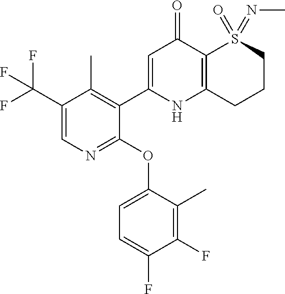 [SMILES: CN=[S@]1(=O)CCCc2[nH]c(-c3c(Oc4ccc(F)c(F)c4C)ncc(C(F)(F)F)c3C)cc(=O)c21]
(S)-6-(2-(3,4-difluoro-2-methylphenoxy)-4-
methyl-5-(trifluoromethyl)pyridin-3-yl)-1-
(methylimino)-1,3,4,5-tetrahydro-1λ4-thio-
pyrano[3,2-b]pyridin-8(2H)-one 1-oxide, 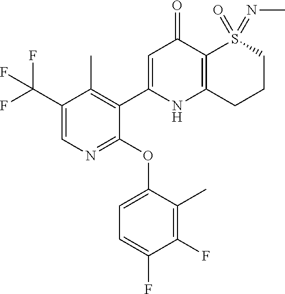 [SMILES: CN=[S@@]1(=O)CCCc2[nH]c(-c3c(Oc4ccc(F)c(F)c4C)ncc(C(F)(F)F)c3C)cc(=O)c21]
(R)-6-(2-(3,4-difluoro-2-methylphenoxy)-4-
methyl-5-(trifluoromethyl)pyridin-3-yl)-1-
(methylimino)-1,3,4,5-tetrahydro-1λ4-thio-
pyrano[3,2-b]pyridin-8(2H)-one 1-oxide, 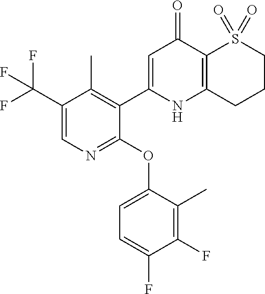 [SMILES: Cc1c(Oc2ncc(C(F)(F)F)c(C)c2-c2cc(=O)c3c([nH]2)CCCS3(=O)=O)ccc(F)c1F]
6-(2-(3,4-difluoro-2-methylphenoxy)-4-
methyl-5-(trifluoromethyl)pyridin-3-yl)-
3,4-dihydro-2H-thiopyrano[3,2-b]
pyridin-8(5H)-one 1,1-dioxide, 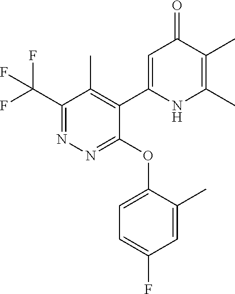 [SMILES: Cc1cc(F)ccc1Oc1nnc(C(F)(F)F)c(C)c1-c1cc(=O)c(C)c(C)[nH]1]
6-(3-(4-fluoro-2-methylphenoxy)-5-
methyl-6-(trifluoromethyl)pyridazin-
4-yl)-2,3-dimethylpyridin-4(1H)-one, 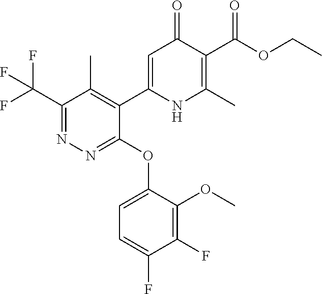 [SMILES: CCOC(=O)c1c(C)[nH]c(-c2c(Oc3ccc(F)c(F)c3OC)nnc(C(F)(F)F)c2C)cc1=O]
ethyl 6-(3-(3,4-difluoro-2-methoxyphenoxy)-5-meth-
yl-6-(trifluoromethyl)pyridazin-4-yl)-2-methyl-4-
oxo-1,4-dihydropyridine-3-carboxylate, 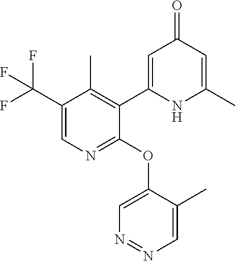 [SMILES: Cc1cc(=O)cc(-c2c(Oc3cnncc3C)ncc(C(F)(F)F)c2C)[nH]1]
4′,6-dimethyl-2′-((5-methylpyridazin-
4-yl)oxy)-5′-(trifluoromethyl)-[2,3′-
bipyridin]-4-(1H)-one, 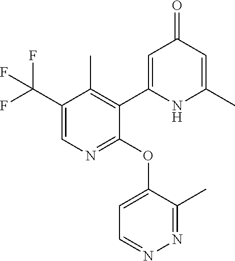 [SMILES: Cc1cc(=O)cc(-c2c(Oc3ccnnc3C)ncc(C(F)(F)F)c2C)[nH]1]
4′,6-dimethyl-2′-((3-methylpyridazin-
4-yl)oxy)-5′-(trifluoromethyl)-[2,3′-
bipyridin]-4(1H)-one, 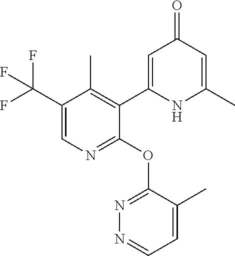 [SMILES: Cc1cc(=O)cc(-c2c(Oc3nnccc3C)ncc(C(F)(F)F)c2C)[nH]1]
4′,6-dimethyl-2′-((4-methylpyridazin-
3-yl)oxy)-5′-(trifluoromethyl)-[2,3′-
bipyridin]-4(1H)-one, 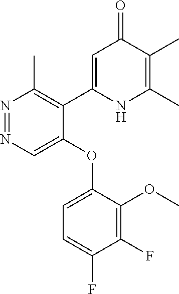 [SMILES: COc1c(Oc2cnnc(C)c2-c2cc(=O)c(C)c(C)[nH]2)ccc(F)c1F]
6-(5-(3,4-difluoro-2-methoxy-
phenoxy)-3-methylpyridazin-
4-yl)-2,3-dimethylpyridin-
4(1H)-one, 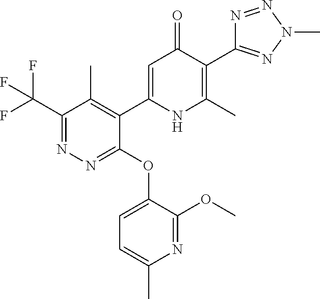 [SMILES: COc1nc(C)ccc1Oc1nnc(C(F)(F)F)c(C)c1-c1cc(=O)c(-c2nnn(C)n2)c(C)[nH]1]
6-(3-((2-methoxy-6-methylpyridin-3-yl)oxy)-5-
methyl-6-(trifluoromethyl)pyridazin-4-yl)-2-
methyl-3-(2-methyl-2H-tetrazol-5-yl)pyridin-
4(1H)-one, 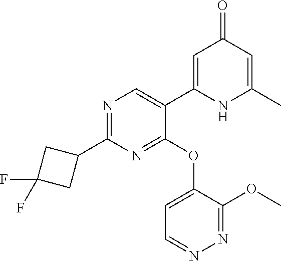 [SMILES: COc1nnccc1Oc1nc(C2CC(F)(F)C2)ncc1-c1cc(=O)cc(C)[nH]1]
2-(2-(3,3-difluorocyclobutyl)-4-((3-methoxy-
pyridazin-4-yl)oxy)pyrimidin-5-yl)-6-meth-
ylpyridin-4(1H)-one, 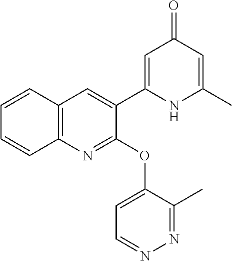 [SMILES: Cc1cc(=O)cc(-c2cc3ccccc3nc2Oc2ccnnc2C)[nH]1]
2-methyl-6-(2-((3-methylpyridazin-
4-yl)oxy)quinolin-3-yl)pyridin-
4(1H)-one, 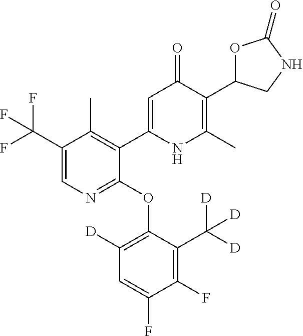 [SMILES: [2H]c1cc(F)c(F)c(C([2H])([2H])[2H])c1Oc1ncc(C(F)(F)F)c(C)c1-c1cc(=O)c(C2CNC(=O)O2)c(C)[nH]1]
5-(2′-(3,4-difluoro-2-(methyl-d3)phenoxy-6-d)-
4′,6-dimethyl-4-oxo-5′-(trifluoromethyl)-1,4-
dihydro-[2,3′-bipyridin]-5-yl)oxazolidin-2-one, 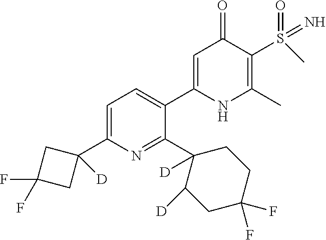 [SMILES: [2H]C1CC(F)(F)CCC1([2H])c1nc(C2([2H])CC(F)(F)C2)ccc1-c1cc(=O)c(S(C)(=N)=O)c(C)[nH]1]
6′-(3,3-difluorocyclobutyl-1-d)-2′-(4,4-difluorocyclo-
hexyl-1,2-d2)-6-methyl-5-(S-methylsulfonimidoyl)-
[2,3′-bipyridin]-4(1H)-one, 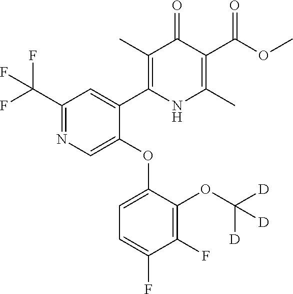 [SMILES: [2H]C([2H])([2H])Oc1c(Oc2cnc(C(F)(F)F)cc2-c2[nH]c(C)c(C(=O)OC)c(=O)c2C)ccc(F)c1F]
methyl 5′-(3,4-difluoro-2-(methoxy-d3)phen-
oxy)-3,6-dimethyl-4-oxo-2′-(trifluoromethyl)-
1,4-dihydro-[2,4′-bipyridine]-5-carboxylate, 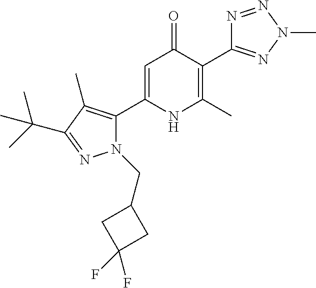 [SMILES: Cc1[nH]c(-c2c(C)c(C(C)(C)C)nn2CC2CC(F)(F)C2)cc(=O)c1-c1nnn(C)n1]
6-(3-(tert-butyl)-1-((3,3-difluorocyclobutyl)methyl)-
4-methyl-1H-pyrazol-5-yl)-2-methyl-3-(2-methyl-
2H-tetrazol-5-yl)pyridin-4(1H)-one, 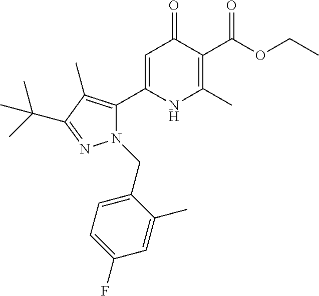 [SMILES: CCOC(=O)c1c(C)[nH]c(-c2c(C)c(C(C)(C)C)nn2Cc2ccc(F)cc2C)cc1=O]
ethyl 6-(3-(tert-butyl)-1-(4-fluoro-2-methylbenzyl)-
4-methyl-1H-pyrazol-5-yl)-2-methyl-4-oxo-1,4-
dihydropyridine-3-carboxylate, and 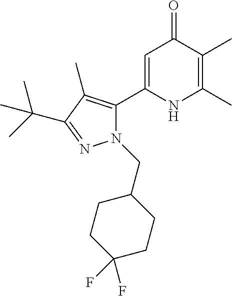 [SMILES: Cc1[nH]c(-c2c(C)c(C(C)(C)C)nn2CC2CCC(F)(F)CC2)cc(=O)c1C]
6-(3-(tert-butyl)-1-((4,4-difluorocyclo-
hexyl)methyl)-4-methyl-1H-pyrazol-
5-yl)-2,3-dimethylpyridin-4(1H)-one,
or a pharmaceutically acceptable salt thereof.

58. (canceled)

59. The compound of claim 1, wherein the compound is selected from 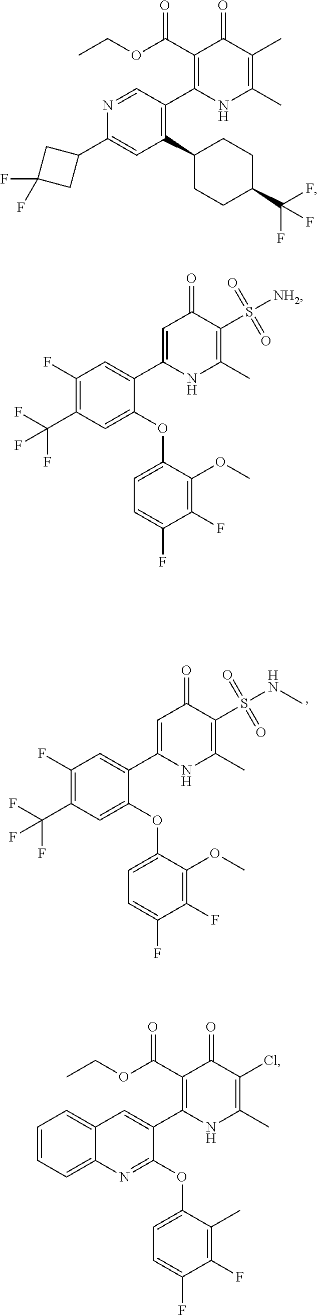 [SMILES: CCOC(=O)c1c(-c2cnc(C3CC(F)(F)C3)cc2[C@H]2CC[C@@H](C(F)(F)F)CC2)[nH]c(C)c(C)c1=O ; COc1c(Oc2cc(C(F)(F)F)c(F)cc2-c2cc(=O)c(S(N)(=O)=O)c(C)[nH]2)ccc(F)c1F ; CNS(=O)(=O)c1c(C)[nH]c(-c2cc(F)c(C(F)(F)F)cc2Oc2ccc(F)c(F)c2OC)cc1=O ; CCOC(=O)c1c(-c2cc3ccccc3nc2Oc2ccc(F)c(F)c2C)[nH]c(C)c(Cl)c1=O] 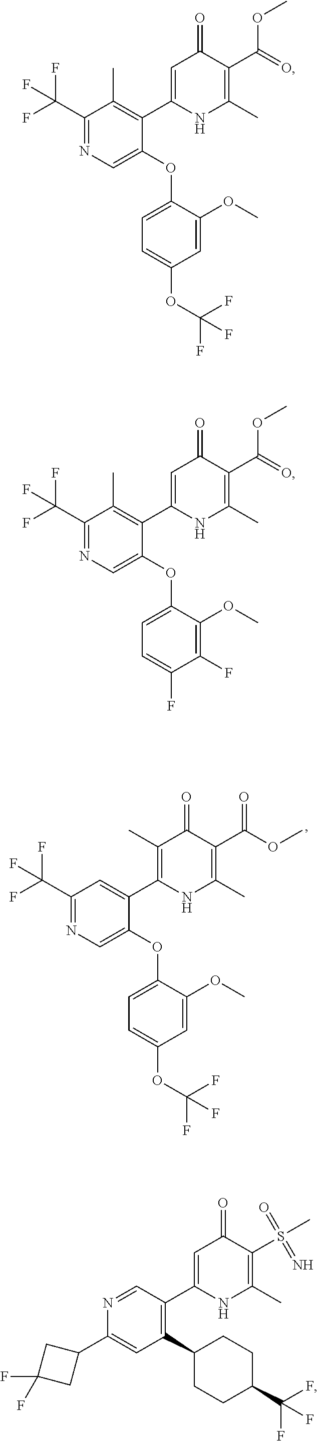 [SMILES: COC(=O)c1c(C)[nH]c(-c2c(Oc3ccc(OC(F)(F)F)cc3OC)cnc(C(F)(F)F)c2C)cc1=O ; COC(=O)c1c(C)[nH]c(-c2c(Oc3ccc(F)c(F)c3OC)cnc(C(F)(F)F)c2C)cc1=O ; COC(=O)c1c(C)[nH]c(-c2cc(C(F)(F)F)ncc2Oc2ccc(OC(F)(F)F)cc2OC)c(C)c1=O ; Cc1[nH]c(-c2cnc(C3CC(F)(F)C3)cc2[C@H]2CC[C@@H](C(F)(F)F)CC2)cc(=O)c1S(C)(=N)=O] 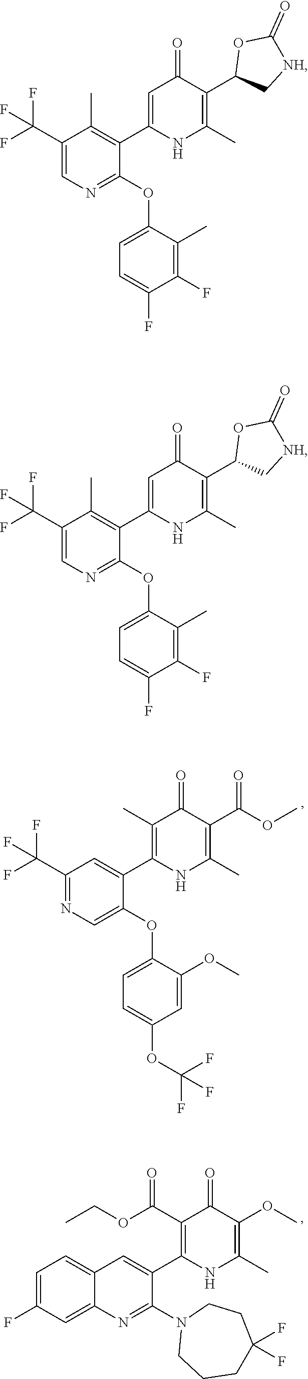 [SMILES: Cc1[nH]c(-c2c(Oc3ccc(F)c(F)c3C)ncc(C(F)(F)F)c2C)cc(=O)c1[C@@H]1CNC(=O)O1 ; Cc1[nH]c(-c2c(Oc3ccc(F)c(F)c3C)ncc(C(F)(F)F)c2C)cc(=O)c1[C@H]1CNC(=O)O1 ; COC(=O)c1c(C)[nH]c(-c2cc(C(F)(F)F)ncc2Oc2ccc(OC(F)(F)F)cc2OC)c(C)c1=O ; CCOC(=O)c1c(-c2cc3ccc(F)cc3nc2N2CCCC(F)(F)CC2)[nH]c(C)c(OC)c1=O] 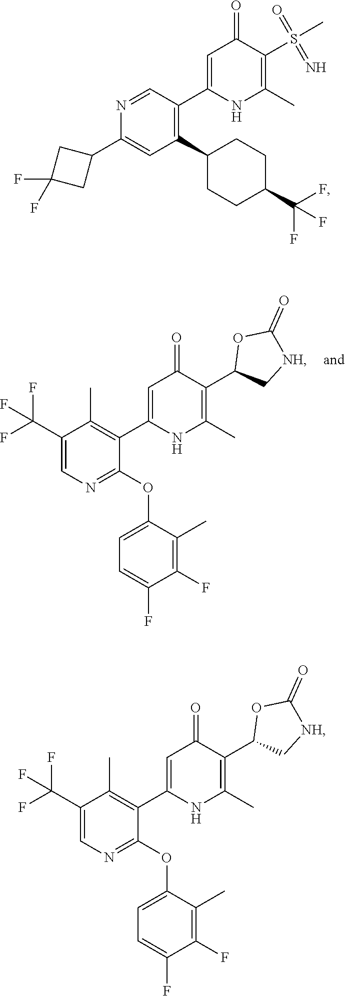 [SMILES: Cc1[nH]c(-c2cnc(C3CC(F)(F)C3)cc2[C@H]2CC[C@@H](C(F)(F)F)CC2)cc(=O)c1S(C)(=N)=O ; Cc1[nH]c(-c2c(Oc3ccc(F)c(F)c3C)ncc(C(F)(F)F)c2C)cc(=O)c1[C@@H]1CNC(=O)O1 ; Cc1[nH]c(-c2c(Oc3ccc(F)c(F)c3C)ncc(C(F)(F)F)c2C)cc(=O)c1[C@H]1CNC(=O)O1]
or a pharmaceutically acceptable salt thereof.

60. The compound of claim 1 in non-salt form.

61. (canceled)

62. A pharmaceutical composition comprising the compound of claim 1, or a pharmaceutically acceptable salt thereof, and one or more pharmaceutically acceptable carriers or vehicles.

63. A method of inhibiting a voltage-gated sodium channel in a subject comprising administering to the subject the compound of claim 1, or a pharmaceutically acceptable salt thereof.

64. (canceled)

65. A method of treating or lessening the severity in a subject of chronic pain, gut pain, neuropathic pain, musculoskeletal pain, acute pain, inflammatory pain, cancer pain, idiopathic pain, postsurgical pain, visceral pain, multiple sclerosis, Charcot-Marie-Tooth syndrome, incontinence, pathological cough, or cardiac arrhythmia comprising administering to the subject an effective amount of the compound of claim 1, or a pharmaceutically acceptable salt thereof,
optionally wherein:
(i) the method comprises treating or lessening the severity in the subject of neuropathic pain optionally comprising post-herpetic neuralgia, small-fiber neuropathy, idiopathic small-fiber neuropathy, or diabetic neuropathy optionally comprising diabetic peripheral neuropathy;
(ii) the method comprises treating or lessening the severity in the subject of musculoskeletal pain optionally comprising osteoarthritis pain;
(iii) the method comprises treating or lessening the severity in the subject of acute pain optionally comprising acute post-operative pain;
(iv) the method comprises treating or lessening the severity in the subject of post surgical pain optionally comprising bunionectomy pain, abdominoplasty pain, or herniorrhaphy pain; or
(v) the method comprises treating or lessening the severity in the subject of visceral pain.

66-80. (canceled)

81. The method of claim 65, wherein said subject is treated with one or more additional therapeutic agents administered concurrently with, prior to, or subsequent to treatment with the compound, pharmaceutically acceptable salt, or pharmaceutical composition.

82. (canceled)
